SEQUENCE LISTING (8 sequences)

SEQ ID NO: 1 - DNA, 82293 bp, Homo sapiens
  misc_feature 1..2000: 5′regulatory region
catcaatgcactccaggctaggcaacggagataggccttgtctctaaaaataacaaagaaaacaaaagaagagagatactgagcaggtagaaggggtactatgatattttggatgtacctgttttttaaaatgattgtctactccaatacacagtgaatccctatgcattataggatgagtgtgctggattccatgacttccttgaagcccatgaattctctgagactgtctccatcatcattgggattgtttggctcatgagcatgaattatagagacttaacagaggtagttcaaccaaggacagagaaaggtctgaaatgcctgttgggtgaggagtggcactgagattagtaaaaaatcactgccttgaccacttcaaaacattataaataaatgtttaaatatttccttaatataatttactaacagataattcgaacgaatgtcaaggaataacacactatcattcagctaccatatatatatagctatatatttcagtttgtgaaataaactagtatttagaacaagcaattgaaagccgcaaataaagatgatgaaacaagttggtaaatactttttctaagcaaactggttgtagatttagcattgtttaaattactgtcaattttttcttatgaggagtgatttcatcatttgtttaaaacctggcttttgacacagtttttctgaagtctttagaaatttgaaaaattatctcgcatgactctgtatttcatgaaataccaatattttaacatttccctgtaaacaataaaacgtactaatatggattatgaatgtgaacaaattgtgaatggactttttaagaaagtagattaattaaatatctgtaaccagaaaatacatttttaaaatgtaggtttgtgtagttttacttctataccttgccacataacattttgtgatagacaaatgctctttgtttagaatttctcttgtagagaaagcttcattcagttgtttctttccctttatttatatgcctgtgtgcatgatgtctaaatcactggtcaggagttccctggggattttcgtatctgactacatggcaagattccagacccaaagtaaacctttcagagagtgggaatgagtgggaggatgtcgccctgggcatggtgcttaggaggccagatttccctaaacctgctctgctgggagccctaggaagcctcagatgcagtctgctgtccaggtatactcacagggtatggggtgggggactgtcaatttctgaatagactttgtctcatccacacgacaacatattttaattttaaaggcgaacgtcaacctatttagaaaactataagtgtgactgatgcattcattatttaataactccggggtgaaacttgaggtgaacggtttcaccctttcatactcaggaatggaactagtcctcggtaccgtgcacagtctgacctcacgctcgcacgctgggctcccggcctcctggcagtgcacttaagtgtaccccgcgtgtgcccggcggactcccgtgcacaccgccgccgtgctgttgagtacgaaagacccccgagaggagctcgcagatgagagcctagctgttgctgtgagactgactgcgggtggcagccggcaagaaggaactgccagtaccgaccctggcgctccccaccaactgcggcgcagcctcctctgcggtgcccgcccttcggcatcactgcagtccctgtagcaaagctaactttaaaaatttaaataaaataataattttttaaaggcccgaaaactccggctcccacccctcccccagcctgcccggcggcggcggcgctcagtgagtgacacggcgagcgtgagccgcggccccagccgggccgagcgcgctgcctgagctgagccgccgtaggtgaggggcccgcgtccccgcccgccctgggcgccgcgcctggcactgatcctgccggtcgcccactgtcgccgccgccgccgcccgcgggcaccatgacagctctgagcgctggggttacaggttggtcaccgccgccggtgcccgggaggctgcggcgcggccggctccgtgccctgcgcccgaggcccgcgatgcgcgctcgttagttccttggccggtccgggccaggacggctcaggggcgctggggctgcgagaggggctgctagaggggctgcgggcgggggccccgcgcgggggaggggtggggaggggcgcctccacccgagagcggggcggtggggtccttgccggcctggccggggcgcgggagccgcgatgcgccctccctattcctgcgcgtgggtgtcgggggcgcctgccgcgggatgcccctgggctggcttgggaagcccagggccggcgacacntngggcctgccccgcctgggcatctagcaggtgttggggggcaggtggctgaggacgaggccactgggcatcactacagcgctgcacctgccccctcggcaccagggccgctgcctgggtaaacctctgggccaacgcgcgggtggggaggtgggggtgggcagcctggctccagcaggtctccgcgggggctccgcggccttgccccacccccatgctcctgtggattgctccttgctaaatgtcccttatgatattcattggccttgttaagcagccgtcattagggtggctacaccgaacgcaaaacagtgtcctgccaagcaacaccattttgtcactgaatctttaagacaccagcaaggatggcctggctgggatccctctgggggcccagatcgcagtcattttgctgacttaatgttcctctttcaggatggtgtgagaatgctgaactcagacccttagtgcttgaaataccttagaataaatctttcagtttcaggtacaatttttcagacactgtattattaaggatgtgtgaatacacgctcactttcaggcctcagaaggttttactcttaatctggtgttcgtgtggtcatcagctaggcccgactgccctggtggtgaaatgtgggttgaggaccagtgaccttcgtgttagtagttggtggataacacctctaataggggatcaaaaattaacaggtggcgattttgaaatatatatgtatgtatatatatatatatatgtgtgtgatttgtatgttcataactacaactgtgatttgatatggcctgggagaggagcggtttcactatggtagttacccctgaggcagatgcatctcaggactggctttttaaattcaaagataaggtttccacagttctaaataaaaatcatggcttcccaaacatctgcaacgagaaacatttactccctaactggttggctgtcattctgcgtgcttttctagagtcgatttttcgttcttctgtagtatactttgacctagccactgctaaaaccagacagtgtgaagacaaaatccagctttggagaaaaacttaaaaagtgaaaaggttcagttggatcctcttctgtgttcatagaagacataatctgtttgcagaataaatgtgccgtcactgataaggactatgctaaaatctctacagaactctaggagaaagcatgtcttataaaatagtggttgtacacttataactttggataggacttgcaagatattcaaaggagaaatgtacttaaaaaatgtacttaaaaaaagtctactcaaaatagtgtcatttagttgcaatttagtgaactgaagatttgagtagcattttcttcttcttgggtatggcatttctttgaaatggtttccaggtgattttatcattccggcatgctttgtgtgtgcatgtatgtgtatacacaaatgtacacatttgttttatcatttatcgatgatgttcagataatttcagaatttccatagctgtataaatttatcccaggtcagagtcacatgtggatttcaagtttgtgaatgaaaattcacatctacacctctatatcctaagataagctaagcactgggccagaagctgggtggtgagtgagaaaaagagataaactgagagcctaagaatnnttcaggaaagacaaatatgtaagcaggtggatatacagtgagatgtcttattttctagttttgctgtggaaactatgatagtgagcttaaacatattaagaatggtccccaaggccacacacggtggctcatgcctataatcccagcactttgggaggccaaggcaggcctattgcttgaacccaagagtccagaccagcctgggcaacatggtgaaaccccgtctctacaaaaaaaaaaaaaattcaccaagtgtagtggtgtacacctgtagccccagttacttgggaggctgaggtggaaggatcgcgtgagcccagagatgtcaaggctgcagcaggctatgatggcaccactgtactctagaatgggcgactgggcacagagtgagactctgtttaaaaataaaaaaagaatgcccctgtcccaaaatgttatcaacattttagagcttcactcatgttatacaattctcgcctgtctcaaggaccagcacagattttcataccttcaaactctatgcaaatgcttaaatttttttaattgattttttgaattttaaagtaaccaaagttgatagagatgatagaatgatagagaaaaaagagtaaacagctagttttaataaccgaagacagatgaccacaatatctgtgtgttttttgtagtctttttaagggcttgacttcaggtagttgtcatcgtggcattggacaacttttatactgtttttaccactgaatatttattataaatatttaaaatatttctatataattttcttacttaaaaattgtaaatggctgtataacaccatacccagtaaatacagcctcatatatttaaccatacctctatcattggacatttgctgtttccagtttttcatcattataaacagtgtcctgatagttataatgtagagatttatttctattttatgtttttcagtataaatatccaaatatgtccatatctgtcacctatttttccatatcttcaaattggtttcctgaaaaattcagattataatttatttatgataaatttccagtaattatgtctttgaaaaaagaagtatctgaattattatagagatatattgtatgtctttaaattgatttcctaaagaattcangattataattttgaataaattgcttaatctgcttttgaggtaaatctacccttttacaaaaagtgttggtggatcacattccctcttttacagaaataaaagaaggtaacataataatttggatgtttgacctttgtgctatataaaaacttgacctttacaaaaatcacctgaaaaccagatgctaaaatatacctcttagagtaaagccaaaataggaatactataaaagttcaaagctggaaaaagaaaaaaatcaatgcaagttcctttagaaaattggctgttgtattcaggagtctaattctgctttttgctttcctactcagacttcaagtaaatgatcaaaagggcttacatagtgagaaatctgtacccccacaggtaaaggaagaagaggatctttctttcctaggctgcagaatccaaggcttttctggtagattcagtatagatggaacaggactgataggaaaagctaaagttcagttcacaatttcctttctaagatcagtgtacaaaaaatacggaagagagaccctggaagagccccttagtatgaaggagcgaagttgaaagttggaagcaggcttttgtaaaacagaattgcttgtttctccatgagatcccagggcaaggcagccataccggaatatgttctgaagacagggactgaccccacaggtccagggtgggggcaggggtaggcctccgcagcaggagtggaagaattagaagtaggatagaccagaacactgaagccaagagaatcatttatgaagagatcaagagtgagaattatttccaattatgcaaagaaaagagatgaaaagaggatgttgtttggagagagaatgatatagatggaagacagccaacagagacacaccaataaatgtttttcctgacagagaggatcacagtgcatggaacacagccatgttttaaagtgagataagattctttttttcttttagattaaaaacaatccaaatcttcagatgaaaataagttatcatgtaccaaaacaattaataaaacttagccaacatctacatatagcctgataaaaactttggatttgaaaaccaataaaaagaaaaatatttcaccagcattgtgatcaaatgcttcagaaattacttatccacaaagagaaaatttagattaatgttatacttcctctctaaaattatcagcaaacataaaataacgtcctgaaaatctgtagtcctgaaaaatatcagtagacaattctcttacgttgtaaatgagtcaaaagtgaaaaataaataggtgaagaaatacatactaggcaagtgatatcagtaaaaaaaaatgaggtagaagtgtgtaaatatcagaaaacagttcaaaaaattgtttgattaaatgagaaaaaagaccgtaaagtcataaaagttgctatctttaatacatttataactacaggcttcccatatataccaacgatcagagcattatagtatgcacagtcaaaagcataatgctggccgggcgcggtggctcacgcctgtaatcccagcactttgggaggccgagatgggtggatcacgaggtcaggagtttgagaccagcgtggccaacatggtgaaaccccgtccctgctaaaaatacaaaaaaattagctaggtgtgatggtgggcacctgtaatctcagctactcgggaggggaggctgaggcaggcgaatcatttgaacccaggaggcagaggttgcagcgagccgagatcacgcattacactctagcctgggcgacagggcaagactccgtctcaaaaaaaaaaaaaaagtctgatgcttattttaggggaaatggaagcggaagcatgcccacagactcaatatctttttaaagagttttgtgacctaaaatggtattgggagtcagcatttcatcttaagaagtacaaaaaaaaaagaaaaaaaaaagaaangaggataaagttttaagaatgaaaacatgaatgaanttcactatgacatagataaactctaaatctgatagcaaaaactgagagctacagcattccaatctgggaggaatattgatctacaggtatatttaaggtaaattagaatgccaacaaattggaatgatgacaaggaggttagtgaagaaatgcactggagaaattggatatacaattccaagattgaagaactggaatcagattgcagtaatggaaatgaagaggtggatggtaaagatatatccacaaaaactaatgctaccacgtcttagtgatggaaggaagagaatgaaagggagtcctaggttaaaaataatttccgatcatcgagcttgggtgattgtgctattgtattctattaagtctagttaaaagaaattgttacggctttatctttcagttaatagtatctacagggtaaatcctttgcttcattcccttttctagttgttttattgttattttgaaattcacatgagattacttccagaggcaatatcgttttgcttcctttctttgaaggcattgaatggcaatttaaaggtgtagtctagactagaaaatcataacagaaaccccaacaaaaactcaaatatcttagttatccttttataaaacaaaaatttaattcctgtatacaatgcaagttagttattttaaaagtaaattctgaagatttctgaggaaagaatagaatatcctgtatatacatacaattataaatcaaaatttttgtctcagacatattttgcctgtgttgcttataattagactctcttattcttttccacacagagccatgactatgcaatgtgaactctctaagtgttctcttctgagcatctaggctagtgatgcctcattaatttggaaacagtgatgggagaggtgtgcagggatcacctgaagatgtttttaaaaaaatacaatgtcctgttttccacccttaaagattctgatttagtatgtctgaggtgaaggaggcctcagcttgagtgtcttagttagcttgggctgctgtaacaaatagcacagactaggtggcttaaactccagactttggttttatacagtctggagactaagaagtccaagatcaaggtgctggctgattcctgtgagggctgtcttcctggtccacagaccattcagctgcctccgttcttcttatagattcgctagtcccattcatggggtctccaccttcatggcctcatgacctcccaaaggccccacctccaaatacattagagataagagcttcaacatatgacttgaggaaggcagaaccattcagtttatagtgttgagaatgtttctgaacctcccttgataattcatatgttaagcagtctttaggaattactattttaaaccatccccttttgagcaaatttgtggattcatattgcaaaatttaacaaacacaaaaacataatctccctttgatcttctgttatgtttaaatatagtagcaaattttgaaaaccactgttttaaaccatccaccttctttatctaacctttacagattcatattataagattaaacaaccacaatcaaaataagtatcccctttctggtttttcataatatataaatgatagtagcagctcatcaaaataaaatatccaacactggataattaatatagagtaggatatatactcagcacagggtaggacggtggtggccaggggctgggaggaggggaaaatggggagttgttaattactgggtagaacgtttcagttatgcaagatgagtgagctcgagagatctgtgttatgacattgtgcctgtagttaacattttatagtgaatgtaaaaatctgttaagacagttctcgtgttaaatgctcctactacaatgaaacaaaaataaaaattactgtgatgtaggtgacaaaaatacgacccagttccctctcatactgcttgacagttttagtttgggcatgtaggtgcaatgacagttttagtttgggcgtgcagggtgcaatgacaaaagggcattggggcattgtggtgtgtactgaatccctttagcaaacccaatcactttaaaggccagagaaaattcaattttcacaattaaaaaataaaacaaaacagaatccttggagaagtaatgacagttttggagtttttctagaaataaccagagaaaaataacctgctgttttactttgggatttaagaaatagacttataaaaagtggggaaccattttttatttctaatcagtaccttgacaaaaggaaaacagaagagtctgtttttggagatggttctttgaaacatttgccttcccctatgtggtcaccaccttgggtagcccttgaagtctgctgggactggaccatgcatgcaaaattctgttttttgcagagctacattatttagtgagggccaagacacagatgaatgagtaccccaaaagaaaagtggcaggtggcatttggaggagagagagagaaggagagaggcagaaagaaaaagagaaggaagaacaaagaaaaccaggcagccaaatcagggtcttgacttttgtacaaaattagattgaaaatcaaccacaacaatccactacctgcaggggcaggggagtgggccgctgttctgttgtggagacacgcattgccgctgtgatgcccagggtgtcagttttttgatgctgtcttagttctcctgctaacatgttctctgctgtgttgctgggggagacgcaggactccaggctaagtttaagagagaaaacatagagctgctgcctttcctctgagcagaggccagattacagggaatgatgcatccagctcattgaagttcacaagctaaggatacgcggaggagaggcttgcttgagagggcccggaaggactggcattgtaagtctagtggcaggcatgctctaatttggttttattacttagacgtggnaataggcaggaacttggagattcccgtctctgttctctgggaaagaagactgttctatttacaaagacttgaaagggtcagactgaaagaagacatggacagcaaagtattcctggggtggcaatcagaaagacctggctccctcgtggtttatcacagctaaccatgtgacctcagacaaggcacttatcttctccaaaactcagtttcacctgtaaaatcagaataacagagagagagacacttacctcattgaggcatggtgagaaataggtaacaattagcctcacacttaataaaccctcagcaaatgttaggtgtaacttgtttaaactgaaaatttttttaatgttttacttgtttcaaagaaattataaataaaacacatcatctttgtgaacatgtacagaagttcgtgtttgtgaaatgtacaaaagttcttaaggttaaaaagtgaaagtcaaatgcttcaccatttctctccctccctctgttctcccaacaaatagactacttggattctaaacttgcttccaattcatgtgggtctgggcatgtttgcctccaatttttaatctctgtagtgggaataatactaaaaactacttcagagaggtgtcaaatgcttagaatagtgcctggcatggagtaagctttcaataatatttgttctcatctttcaagttaaaaaaatgagtacataatgtctaagcttgcttctattctataattatgcacagtagtttatggactttcattctaccctgagaaatgatcaaatgcataaaaatctgagcagtgtcaagagtattaatcacttttatcattggaagtcactttggatatcatctattgcaaagtcccagacagaggagaaatcttttctgcaaactctaaaatgtgtagtcattccctctgagagtatccataaaggagtttaccaccttttgggatattccaacatattatgattggaaacttatagagtagatgttctttttatcaaataaacagtcacctcttgataaacttttgttagagaatttctattttgtctgcattttccttgctagaagaacagagagcactacaaaacaaatcataacaactgtgttctgggctttggattttgggtatgacagaaaaactttctcaatacataccttttaattgctcttgcacaatctctgtagaaaattacatacattcttttagccatgtatcataggtggaatctattttttagttttcatattcttagcctagtcagtcaagaaattacgagactaaaactaactctaggaagtgaagaaatctgtcattttaggaaatggagagatctgtctcttcactacatggggtgagattatcaaatgggagttagtaaatatgtttttctcattctttaccaaatttagagaaaaaaataatgaaggtataatagaatatttctaggattcatcctcaaggaaaaaggaataataaatggataataccttcagaaacttgaaagggttatttagatagatttaatgagaggtttgatgatttctaccagaaaaaaattgacatttttataagtgtgtggtgagaaaaatcataaagtgagtcagataataaaaatggattcaatttcaggagatgagaaatgtctgtactatttgcacaaaaaataattgatgccaagagtttgtaaacaccaggctatatgatttaaatatattgaaaaagtcattttctcatttcctgtccattctgtaatatacttaatttttgattcaatagttctgtcaggcacattgtcccgtcttgtctctgtgcctaagtttcctcatctgtaaaatggttcttgtgaaaatggaatgacatagtgcatattcttttttctttttttctttttgagacagagtctcactctgtcacccaggctgaggggcagtagtatgatcttggctcattgcagcctctacttcccaggctcaagtgagcctcccacctcagactcctgagtagctgggactacaggtgcatgccagtactcccagctaattttttgtatttttttttttttttgtagagatgggttttctccatgttgcccaggctggtctcgaactcctggactcaagcgatccactgcctcagcttccctaaatgctaggattacaagtgggagccaccacacatggcctgaaaatgtatgatattattaaacagactggcacataataacccctcaaccaacatttttgttagaaggactctgtttttgcatgacagtaaacgtaatttgggttgaaatcaaccagtgagctcactattttaacatggaaactctcatgtatcccaaagcactttttcatggggtatatgtgtgtgtgcaagtgtgtgtgagctactgaaggctattttcctttggagaaagcattctgagtaggagataacattcatttttacangcaacttctgtttttaacactttcccttaaattgtcgtgatgattgaaatctcaaggcangtcawtgagcnagcccctaaatgggagacagcagagttgctggattctggtctggaaagtgcccagtgcgtgtgagaggctgtggaggccgtgagaggtgacctcatggagggagaggaagagcgtgagcatgcaggacacagatgatctgggctcagagaaagaggggaagttcatacactcaagatgcacagagaggacactcagctgggcagagggaggtgcaggtatcagtagctctgaggctcaaatgggccctgggaattggaggacggcagagtgggcagggatggtgacacatgaggcaggtggatggatgttggataacagataataagacaaaaatatggggtattttttcctagttgaaaccatagcataaaatgtatgatgctacattcatttgctctgaatggacatgtcttctgtctgagaaggaacttagcaagcaaaattgacagtcaaactttacagaaacaaggccatcttgctgttaagcatcgccttttgtcgtcaagttttcactgtggtgtattgaggtaaaactgaagatgtaaatgtgcatatagggaaaggacaggaagctcttgttcaaatgtagattttagtggtttgttgttttgagttctgtggctattggaactaaaaagaggaagaaataaaaacatttgaaataagaattaatattgtaatgcctacattaacaaattggtaacatcaaatacatgtgtcttcaaaatatggggtaatgcctttctcagatacaaatttaacactgggaacctaatctggaaagctgtgcttatatcagctcaccttgtaaggccttgcacgtataagcatgtaaatgagctatgggacaagaaagacaaggaaggggcacagatgtttgaaggtagtgtttacttacagttagganttttctgcacatgtgtatttattgacagaggatgatgttcacagaatatggctagatttaaaaaatctggttacaaaaccatgtgtaagcagccatttaaaaataaatgtatgcatggaaaaatatatgtggcaaagaaagtcctaaaagagatacaattcatatcctcttggagagtaagcaccctaaagaaaattccacaagagaccataactaattacttcatctgcagccccttgttcggtggcagacacaatgtatgtgctcaacaaattctttgcaaaaatgattgagtctctagaagtggaatccctgatggctttcatttttgtttcttattttgtttatatagtttttctaatttttctacaacaaacctgtattacttgtgttaatgaaaataaaataagaatagaattattttcttctaacttaatttaaggtttaagtatttgagatctaggcaaagaaagaccagtcagctctcctggatttttgcatgaatctcattatgcccgatagcacagaaacrtgttgggaatccactgctcaatattccttgcctagggaggtagtatggagccagcagtaaggtactgaaggaataagaaaaccactagatggacgttgaagagaaagtgcgccacactagaggctctcttttaattaatcagctactctgcctttaaatacagtatgttgtcttccatgtcaggaacatggagggaaaagacctagtatgttttgttcaatgttaaatccacgtcacttaacaaaatggtcaggatagtcaaaaaacaaaacaattatgcaaacatgaatgaatgagaaacttctgctagcaatgctggttggtttcccaaagacatgaaagtcagagtttgaggtgaagagtcccggagatgttattgaacattttcttttcttaatttccttctcaaactgtgccagggatactaagcaaagagataaaaacccaccattaagtgtgtatgtgtgtgtctgtgtctgcaatagcgcggttgataaacaatttcacttgcactcctaaagaccccacagtttatgcacaaaagttatcagaagagtggttgttggtattgatgtattgacaaccttaataacacctaatgattaaaaatgagtcaatgacttctgtcttctttggggctttttagaatgaaatagatgctttcattttatacaagttgtatgtttatcttctcacatctttattcccaaatattaagtttttcagttataacaaatcacacacatagacaaagtcttcatgagttaagtgagggaattaggaaagactgatgtgggattggcattggataacaaagaggacatggcttggataaagggtttgaattagttgagtaaaggttgcaggatggtgaataatggtatgtttagccaacctgagtgcagcatgtggtatgagtgtgaaaggaagggcaggtacaaatcagtgggactcagggtcctggaaacataaggttaaataaagtaggcagttcgtaagcttcaagaatgccttcatattcagtgctgtatacccagcacataccgcagtgcctgataccacagtggatgcataatatttgctgtgtgaatgaatgaatgaacaaatgagtgagtgaatgagaaataagaagatacactgaggttttctgggctacaaaggagacctacagtgtgtttggaagctgatttttttttatataaaagagggaacattttgaaaggcatcccagacntgagggcccatggcctacntagaaaggagtctagnaaatggggataggtgaaaaaggccgnttgtacatatatggaatgaatgagcnaagaaaacaatccactgctgagagtgggtgcaaagagaacctgctgggccttcccgctgcacatggggaacctgaagagtttagagcagtagggtctgggacatgagaggaaggagaaaagcaggatgcctggctcatgtcgctctctcaggctgaatcttcattcatgatggtgttggcaccgtcttcctacatctgcgggggaagcagcaccgatacctctctaaattagtagctggacacagatggtaatggaaactgtaacaacaggtgatcacttaaggggttaagtagagaagacaggcaagaagggcatgctgtgaaccataatttggaagacaggagaagattcaaaagactttaatactgaacccaagagagctgcaagggaaagaaatgacccatagtgttgcaagggcgctgcagttaaagaaaatggaatattttaattttaatactgtgaagacggggctggaatgaagtagggaaagtatataccaatagaaatgaaattgctatcacagaatacaaaaactgggtcttaaccatttaaaacagcaatgaacatgggatgaatgagtttcntgagttgctataagctgtttctgagtaaaagatttattaatggatacattgtgaactgtgtagcctctcnnccgatattgaantatttaaataccaactacttttaataaatgtcggaaaccttttgaaagccatgtttttcaaactttgtgtactgtcttatgaaggacactttggtaaacagaaaaaagagatattggcttataaaaacataataatgataaggctcagatacacaggaancaatttcctatcaaaataataaacttnacctaggatacttaaatatccctactggtttttatcagagaaataataggtctgctgtatgcagttttattgattcaacatgaatgaaggcataaaacaatatttctgtttgcaaaggaaaattattacaatcaaccacctcaaattaattttgaattcctcatgtgtttgtaacttaaatttattttactacttgaatgctttatataatatttaatagtattaaattaaatctagagatccaggagctagttgttgagtttttatgtatgtgtggattagccaggtatctttggagcttagatcatttcttgggcactgtgagatttagtggcacctgggagcaggtaaagtgaaggtaaatgtgagtgacagctaatgtcattcagttataattgcactattttggttggtcagatataaccaaagtgggaattttttaaaaaacaataaattctggacatgtatttttctagataaacaaatccataatcaaaaatgatttttgctttaggtggcgtattgataataaaggtgcatggaattcatatgcccattgtctccactgctcagtgaaagaggaattgtggacagatctctaaaattccttatataatctttgaagtgaaaccactcttttacttgctcctttgtatgtgtcatcttgaaggatttctaaaagattgtatctacactcagtatttgggtggataggagactgtcattgaagaggcatctcaaattgaggccaccagatttccttccccactaccagtacatcagagaaaaaaaaaacactttaaatactctgtatacattaacatgggatatcaaagagtcccaaactccaattatataacataaattaattagtgcagcctactgcaaagcccatattcaaactcaatattcagagcaataacagcctggaaatacccagcatgtcttcaagccagttagcttgttttcttctgggaataggaggtgaatccgtccgttttcacacttctataaagaaatacctgacgacgctggctaatttataaaggaaagaggtttaattgactccattctgcatggctggggaggcctcaggaaacttacaatcatggtcgaagacaccttcttcacaaggcggcaggagaaagagtgtgtgaagtaggaactgtcaaacacttataaaaccatcagatcttgtgagaactcactccctatcatgagaacagcatgggggaaaccgccaccataattcaacccctctctccacacgtggggattgtggggattataattcaagatgaaattttggggggtgggaggacacagccaacccatatcaggagagttaggcagagatacctgcccaggtagataaaggagcacatagcgatggggataggtgctcaatacaggcaatcagattcggagctgctttgttctgagtttctgtgttttattctcttttccagtaaaaactgcatctgtgngcgcgcgcacacacgcgcgcgcacacacacacacacacacacacacacacacacactgcaaacatgttcttgtactccagtgaggactaagtggcacccattgtgcttggttaggtttctttctctagacccatggagcaccagataggagagccagtgaacccgcctgatgttaaaactgcagtgctacgttttcctgactcagcctcttttttttgcctgaagcgctcttttttttctgttacccgccttgagaaattctcctaatagtctgcagcttcagcatggtcaactctaaaccccttacacattgtagtcaaacagagcttattttaaatttcaagtttgcttggatggccttggtcaagttattacctgccttgaggctagcttttctcatttttctcatgcatgtgtaagaagatgagttttttgttttttgtttttttttggttagggctgttgtaaagatcaaataaaatatcattgctaagtgtcccaaacatagtatatgcccatagctgttagttcctcaccttcctttaagactcaatccaggtgctaatcaaatgggaagtctcacagtcgactcagggatcatcaggtaccacctgcctttataacaattcttctcacattgtattgcacttatttgctatttgtgtcctgagggctgacatataatagccccccttcaagtgactgctgagttgtggaaggcagtagcaatggccatagggaggtaacgtctatcacaggatgtagatgtttcagaattgtggaaatttaagcagttagcaggcatctcatctcccacctcaatgtaatttcatgaacaatttacttcagagtagctggaagaatttagataatcactgcattcataaaacaacatctccatttaggggaggttcttcccccattctttctgacctccctcccctcccaagttgggagatcaacttggggctactgttggaagtaagagacgtctgtgaatgtcgtattaaattaacactggggaaaccatggggaagatctgtgcatttcatctgtattactcagcagtgttctagcacaaagcacctctatgtggagacagctttaggtataagagcatacatctaaaaagattttcccaagaatatgtctgccctaagaagcaacccattttactctagctattctaaatagctagcctctaaactgcttttgaagtacataaaataatccattgctttgggagaaaaaaatcaagcttctgttggtattaatttatctcatccttttaaagtttctattttcatatgtttattatacactatggagtagaacacatatatgccttacaaataagtagatatatgtggggaactcaattttttgatgagggcaagatggtttagatgacaaatacaacttttccttttncccagactgtggttttgtgcttgctcaccaaagctaacctcagcatgctcaaaaggaagcagagttccagggtggaagcccagccagtcactgactttggtcctgatgagtctctgtcggataatgctgacatcctctggattaacaaaccagtaagtttcttctttgagtatggagaagcatatgtggatgtgagataaatgtgattgacaaattcaaatatcatgtacctttggcttcgnaaatatctgagctgcagtagaatatggatgaggttcagagtctggaagtagagaacaatccaatgaaatagtgagatgtcgctatgagttactcacataggaagatgcactgggtagtctgatcccctcccctccccacgctgtgcaccctgtcctcagagtcacatggtgtagagatgataccacttctcaggctgatccaaagctggggtccaagatgagcaacctgttctgaatgaacctcctttacttgttgtcaacgttcctgaatccgtgagacaatatgcacatgtttggaattaaaggtctccttctgatttgcaaaatttgagtaaaaggtcaatgagataatggatggtatctctgaagaactttgtcaccttcaccgcctaattaagttgatgaggatttgagaagaactgctaggccaggcacgactgtgtgccagaaagcatagtaaccagaactaagcctgacaggcatgcatatccatgagtgctggaatctcagctctgcaaaaccaagcaggatcaacttcttactcatctcatggttgatgctgaagtaacaaaaggaaatggcctgttccctcttgcagtgcttgtccttcccccaaccatgatacacacatgatctgcttggactctttgctgtccttcccagtctctcttcctctaatctgccctccaggttactttccctgcctgctcacatttctttctatatttctatttttattcacatcacgcttctgctttataatcatggaagcatccctattaccttcattgcaaattaaaatacgttagtgtgacattcctgcttcttttcattcaggacctaacctgtccagcaactgctcctttcctgtgtcagcacactgtccacttgagccccatcagcagcctcctactcctctaggctctctggattgttcacattgctttccctgctcgtactgctccctggccccagtgaccctttcccagctcctctgatgagtcaaatcctcctcatccattaggactggatgagatgtcacctctggcatcctcctctgaaaggcttatttccgccacagacagaatgtacctctcttctctttccacttagcctgttttatatttgtatgtaaatataaagtgatctgtaaataactgtatttttatatgtatcatatattatgtgtatatttacattatatacattgtaggtcgtgtgtatatttatataatatacatatatttatggatctattgtcttaggtatatatacatgttatatacacatacatataaacatataacaggnttatatgtatatcacatatatacacattacacatacacatatatgttatatgtgttaacatatatgcatattacccacacatataaaatatcatgtgtatataancatatatacatgtcgcatatgcatatacatatatattatatgtatgtataatgtgtacatatatttatatgtgcatgcatataacatgtatatgtaatatgtatatatgttatatacgtgagactatacatgatatatgtatattatgtcttatctagaacatgttttataatgttatataatgtgacatgtttttgcctcttgcagtgcttgtctttcccctaaccatgatacatgcaggatctgcatggactctttgttgtccttcccagtctgtctttctttctctaatctgccttccagactacatgtgtttacatgtatgtgtatttacatgaaacataatatatacatacatataacatgctatgtgtatatgcacacattatatatatgtatgtgtgtgtatatatatatatatatatatatatatatatatatatatatataatacattcataggtgttttacttcttggactgcaaagcaggagtcagaagtgtgttgcttccatattttgttctccattcccagcattctgacacatagtaggccttcattcatacttagagaattgaatggaatagagaatgaagcacccctaactttttcttagtgtccatcttattacagagctttctacaaacttatgaagaaggtagtataaaagatgtggccttggaggcacttactaaaattaataattcttcattaaaattatgtctttaaactaaaaacacttttgttgttgaagaacttatatttggaagctcttttgtataattagattctttatgttgaaagtccagatggggctaaattcacacaccaatgtttattgaagtgcttccaaattgcatgaaaaatacatcaacctttaaagtaaactgaatcatcagtggcttatgtggtatggaattagcagcaaaatatcttcatgtaaatattataactgtcagataaatattataactcctcagctgcaagtgtctgagtctcctgaaagttctgacagtcttttctantttttcatcttgagtgtttngctagtatatatagnctgcagaatccatctactacaagactaaaatttttttcagagtattatattaaagaattatcataatttcatatatgtcccagatagtcaataaaaacgtatttggattatgtttaagaattgtatactaattgattaactggtaatattaattgtggtgggtttagcatggagatgcagttaagtatttttttaatcctagatactagttttcataaaaaaatattgagtatgagtgattgtatgtcctgtatatatattcatgttaacacacacttatctttaattctttcttcttattttggggttaaagaaaatcttcagacagcagacatgcctgtagtgttgcacagaaagttattttgtggaattttgaaagatgtctgggctaaatttgtataaaatgtgttctccaaaataaagagaaaaatctatgcatttttttcaaatgtaggatgagaccagatttttaaaaagtttctttgtatttttatacaaaggcattatatatcagatacaaatagaaaggaaattctaaatatgggatgtgttatatatcaattcatttaaaaagcatttcttaactactatgataggcccttgtgctagatgctagggaagttatggtaaatatagtatgcccctaattctaggtacttatagtacaaaaatgaaaaaaaagtaatggcacatcaagttcctatgtgactaaattacaaacacatattttaattgaaccagaatcttcctatattgtagtctgatgcccaggaggttatcagatgtttgggagagcctgatcaggttccgtctatgctttgggtctttgtaacgcatggccacagaaatgcctccacctttctgtcttagtgtgtttatgtggaaaataggtgccttgagaattaaatgatatnaaaaacgtgaaaatatttgtagcacattatatgcacttagatgttagatttcttcttcttcacctccatattagtaaactattttaacctcagtattatgttttccacttagtgtggttcataatacaagtttgagatatcagttatggtgccgtgtagtaactctggaccatcattgcagtgggttcactctttgctgcgcatctgtgccatcatcagcgtcatttctgtttgtatgaatacgccaatgaccttcgagcactatcctccacttcagtatgtgaccttcactttggatacattattgatgtttctctacacggcagagatgatagcaaaaatgcacatccggggcattgtcaaggtgagcacttccatgtcatttaaactaagaacctaaatgtgttgaaagtcttaatccctttaatcatattttcttgttcatttcagtaaaacaaaacatcattttatcttatacatatggaagtcctttttaaaaaggcataactccagagactgggagaaattgttgcatgagactattttaaaagttaaataaattttgtaagtcacaaactcttggaatatctgagaaggaacaagatggtttgactaatgactgtgtgaagtctcgagtagggttattcatgccacttagggctgagcccacaacccagcacaggggctggcccagagaagcaccttattatttgttgacttagggagtgcagaatcagagtcccatgatagagttagagcatgatattattgcagcacgatgtcatttacttgatgtgttttgaaaatacaaaaggataaaaaaaaaaggacatgagatggaaaaaatgtctggggccaaaaggaaaattatgtgtcatgtgaaattagtaccaattggaaacaaaatagatataaaatgttttaaaagggtaaagaaagggaaagaaaaggaagaaaatttctgtcactggatatcttttccttccatagcactaatctttacataggttaaacatatgtagatgatgctgtccctagagtatttaaattgtactatttggggaagcagtaataggagtgcagattttagagtttgacaaatgacagacgactcagatcttagttctttcacttactggnttgtatcagcatctcaagttagctaattttggtgagaaagtgttttattttgtacaaggattttgagaatctactgagacaatatataacacataattatttgtcaatttcattgcttcccttttatacataattaattgtgtgccatccttgaaagacagagatcacagacaatgcattaataggttcttgtttaactttttattgtggactttaaaaaatttaccccaaagtatattacagttataatgtgaatccctgtaactacacatcaacaattatcaatatttagccaattctgtttcattttaaagcaaatctgagaaaccaagtcatttcagaaataactccttttgtattggtgactgataaaattttctctttttataaccaccataccattatcaaatataataatattttacgtatgataaaatatttgtaacaagacttagcaataattccttaatgtaggctaatacttaacccaccgttcatatttcctaatattgtcacaaattttttttatggttggtttcttcaactcaagactcagacaaggtttacatagtatacttgattgtttttgttttcgtttttgggagggaggttcacttttgttgcccaggctggagtgcaatggcacgatctcagctcactgcaacctctgcctcctgcattcaagcaattctcctgcctcagcctcccaagtagctgggattacaaggcctaagccaccaccccgggctaattttgtattttcagtagagatggggcttctccatgttggtcttgaactcccgacctcaggtgatctgcccgcctcggcctcccacttatgtttcttaagtctcctttaagtctctgtaagtccactaagtttcagcttttcacatcctgcaaaaaattttcctatgtcattgatccattggaagacatgcgtcttttgtcttgtggaagattctacatttgagatttggctgattgcttccagatggtgctcttgaatagtttnctgtgtactccwtgtncctctgaactgctttngtannttttgaccagncaatgcacacaggtgcttctttncccacatggtcacctcnaaaatatgtcagacttctggatatttgccagtctgaagggtgggaaacggtaatccactgtagttttaatttgcatttcccttttgagagtgaggttagatgggattttatgtatcaagagccatttttaagttactattttttcattcaattttggccactctgtgtgtgttagaagtatcagctgtttgtgatataagttgcaattgtttttcccagtttgccattgtcattggctttgtttgtggttatttgtaccatacacaattcttatccaaatcatggacaaactttctattgtctgcattttatgtcatacaggagagttttgtaggtttcctcatacagattttgcacattcttggttatatttatgctttggtattttatttaatcattttgttatttgtatgaggtcttctattacatgttctaacttgttcttttaaatgaaggctgttggtttctatacattaatattatattctgctactttactaaatgttctagattcttgtattagtcataatagttttcctgctaattcttttgatgcttccatatgatcatatcatattcaaataaagaaagatttgtctttttctttgtaatttttatgccctagttgcttttacttacttaaagtaaaataaatgaattgactaacactgccggtaaaattttaaatactagtagaggtagtgaacatttttattttgttttgtttttggtttatctagaacatctctagtgtttcttcattaactagttcaaaaaattgggatttgaagtatgtgtacattattggattaagtgaataaactttaatttcacattttgagagttgtagcacaactagctttttaatttgtcaaatgtttatttttaatttgtcaaattattattttttccttagtgcttgtaagagaagagatttttaaatattgagttatccttccattccgataaagaaaaatccctactgggtcaacatacatttttattctattttaatgtgttgttttgttatatatgttattattttccttattttaaatttggtctaatctttgtcaggttttgatattagatttttcatgtgtgtgggtggcgggcatgtatgcacactataaaatagtctgagcactctcagaattgttttcactttaaaagtttggtatcagttcccggtgaaatcatctggtcctgttgcgtttttgtgggggttcctttgcagtatttcctatttcttctatggaaattgtctgttttatttttatttttattttttattttttgttttttttttgtttttgtttttgttttttcgagatggagtctcactcttgtcacccaggctggagtgcagtggttcattttcggctcactgcaacctctgtcccccaggttcaagcgattctcctgccttagcctcccgagtagctgggactacaagcgtgtgccaccacgcctggctaactttttgtgtgtttagtagagatgcggtttcactgtgttggccaggatggtctcaatctcctgaccttgtgatccacctgtcttggcttcccaaagtgctgggattacanggcgtgagccactgtggcagacctatgtcttcattttttagtagagacaagatcttgctctgttgcccaggctggagtgtagtggtgtgatcatagctcactgcaacctccagctcttgggatcaagagatctcccacctcatcctcccatgtagctgggactacaggtgtgtgccaccatgtgtggctgattcttaaaactttttttagagacagggtcttgctatgttgcccaggctggtctccaactcccagactcaagcgatcctcccacctcggcctcccaaagtgctgggattacaggcgtgagtcacagtgttcagtcaaaactgtctgtttcagacttgcttggtctattggggtctatttggtgaagtatattttcttaggaaattatcccctatttattttgtttaattatttggtaacatagacctgtgtaaatagcttttctttcctctgtttgattttctcctaattttgtatttttttgttattttggttagcattttgttgtcatcaggttggctagcagtattttttgtttgttccattttgttttaggttagccaattggtttgctcattttgttgattttttttaaaaagaatcagcttaattaacttattaatttttgtttaatttttttcctttaaaaatatttttattatactttaagttctagggtacatgtgcataacatgcaggtttgttacatatgtatacatgtgccatgttggtgtgctgcacccattaacttgtcatttacattaggtatatttcctaatgctatccctcctccctcccccgaccccatgacaggccccggttntgtgatgttccccttccntgtgtccaagtgcatttattagtttctttctttgctttatttggtttactttatgggtctgtttctaacttttcgaaggtgtttgtctcacattttcatttcttgagactcttaaatctgctgcttccttgcctcgtggttaaaatgttaaccacaaggataatgttaaccagcagcataaaatgttactgctgagaatgttttttttccctcgaagtggcttgctcttttctcctgaatacggaaagagactttccctttattgttcaagtcagcagctctatgagaatgtatgttatctttttgttgatagttacggatatattttcctagtttcatcatgtgatatttattttattgcattgttttcaggattccttttgtattctaattataccaggatctatggtttggtctttcctttcctttgttttctttccatgtatttccctctgtttttatgttttctacattctctttcccttgcttcactcctcatgctgtcccctcactcctctgtcccctgtatttcactcctctttctcaaatacttttttcatttctttttcatcatttcctgaacagtttttgttctttcttttttcagtctcttatcatttagacatcatttctgtgttttcttatttctagttttgtgattcttttggagcttctatttgtttaaatttcttttggtcatgttgtcatattcgattatagtcttcatctgctttgagtgtttattttctgtttatacgtttatgtattattttatttttacttttattattttttgagacagaacctcgctctgtcacccaggccggagtacagtggtgcaatcttggctcactgcaacctccaccttctgggttcaagcaattctcctgcctcagcctcctgagtagctgagattacaggcacgcaccaccacacccggctaatttttgtatttttagtagagatggggtttcaccacgttggccaggccagtctcagactcctgacctcaggtgattcgcctgccttggcctcccaaggtgctgggattccaggcgtgagccaccatgcccggcctatgtattatttcattcatcttattctgtaatgtttttacaagggcttcatcatgatccgtttgtttggttgttcttcttgttgttcacatgaaatgggagtttttcccacacattgggtagagtagcctctgcatattgtcagtacgaggggtccctccactgttgttatcctgatgggtaaacatggagcttttctgtgtaccgaccgtcaggttctgagctctgctgcctctggactgctggcccaattatcgaatatttcttacccactggccccagtgtccctgccctactgaattgccattctaaacatggggctgtggtctggaaagggtttttgttgggtgtcctttggagtttctaatattccattgcctctgatctttctgcagtttcttggggacattggtagtcatttgtttttgttttgttcctttgttttacattaacctgcaacttgcagcctaccagtttacatttctacaaatgttgccatttattttaaggttcaccttctgagatatctgctcagcattccacacgtatttattagtgattttggggttttggaaatctactggtttatggaagaggtgggcatcctccatagcttcaatgaacagatttcaggatgattccaaaattgtctgtatcttccacaatgtggctaggctagtttttataagcactaggtttagataangcataggttcagaaaactggaacctgtctcaggaggccatcttgggagaaagttggtcttgggagaatgctaacatctctgttattcacagtactcccctggacgttgcccggccctagctcacattctgtgtagttgcccggccctagctcactagagggccatgaatggccctgtcacttgtgtgtagtggttattggggccatggcaacctttgcccatgcattgaaagatctgaatattctgggtgaagcattttatgattgaaattgccatgtgttgacttcttaagggttccatttgaatgaaacaaataagtgacatgttcctacaagaaaacttttgtttttttaagtcattaaataaagacttagatttcacatcactaaaatgatagtaggcattctgctagttgtcacagtctaatgataaaaagaacagaggcatacttaatatataaattatatactcattccaagtgaacagagcagtgagagaacacaagttttaataatgtaaatgttagaagttgaatcaatagttaaaagagaaagaaatgacttaggaccaaatcgttttcaaaatgctgtaaattagccatttccagtattttcacatttccatcaacatttgggtgatgagctatttactacaaccaaaaatttttttttttattttcaatctgtaagaggtgaaatttaaaatgaaaatcaaataaggttttctggttttcattcagaaataattggatcaaatacaactgtaatgcaattttagtaatattgcactgttaacattatcttgaagtagtcagctcaacttcatagctcagtaaaactggtattctatgtttaaatgctagctaacgttttagttaaatatagttaaatatgaaatctgtttatcctgaaccaagctttaaatgtatcaactcactaaatttttaccatcatggcctctattattgttagagggtaataattatatcagtaagtactgtcattttcatgatattaaatctaagccacagttttattatttttatactacagacttcttcaaacatgcatgtcataacagatacagctcaaagagtatattgggcttgatattattttcttatattcaaaacttaaacttttaataagactttattttagcaaaatgtcctttgtcacattaaatgtatgttcctaatttatattttattagctttgcatttttgtatttgtaaatttgttttgcttttatacttacatgagaaatttgtacatattgtaggtgtttaagttacatcataataaaaacacactgtcacatcgaagttctatacctttcaggggccctctcatattactcaagtttgagttactatgctttcttcaactctaccataatttttaaaacttttgttaagcacttgtctgctgaaaacctcatcatttttcatactttttctaccttaatacatttgccaatagcactgcagttatctctcaattctttagtcccttncacccagaataatttgcaaatatgtgctgcattttctcactaacatttaagcttgtattttttcatacataaataggaacgttatgaatatacatgttctattttaatcttcaggttacacttttcatagtaagttttcacattgtttggaacagagttgtgcaaatatgaaatgtttaatattcactgatttccaactaaaatatttctaaggaatagtcaaatttagttattgagtgagcagtacctgttagccatgtgttctgggtggaaaagtgtgtgtgagttgatgtacacatatgccagtgatttgcgaaatgctaacctgntantttttttttttttttttttttttgagacggagtctcgctctgtcgcccaggccggactgcggactgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcctggccaacctgttattttttaaagagtcttttccatgcataaaactatgttagtccataagaaaatatttatagaagcaaaccaatgtattttttgcccagctgtcatgcacagttctttaaaattatacctagccaattttggcaatgttatggtgtacctaagagtcatacctaatcgttaaagtagtgttatccaatgatacctagtttataaaattatgcacgacaaaccaaacccagaaactttattttttcaaaacaaacgtgtcaaagctgtggcttcctagcattgcctactaagtttattgacctagtaatttgccaaatatattaaaatatgtataaatataaatataaaatatgtaagatatgaaaatgtttaagatatttaagatataaatgtaatataagaatgagttatattcttaaacccaagtacagtttccaagttttcctttagtcatccagcaaccatttattgaacacttacaatgagctcatctctgtccttggagaagggaaacagacaaaaccaagcagagctcctgtcctcagagaaaacctggttcactctgtgggacagacttagcacatacaggcactcttcattttcaacatccaatatatttctgaaaacatggtggatagcatattttcagaaaataggcacttctctttcactaatttatatgggaaaactaataatgtattaggggagctcgactgatttctccaaatatcactgcattaccatctaatacctatacaagtcaacctgcatatttctgcaaaataaaaagcatttaaaacatcccttacccaattatttgcactaatcaaaccacagtggatgttttatatgtgcccaacaataacaggaagtttctgttattaagtaaatttaaaaaatacattaaagtaccatccaccatattttattgaattgttgatatcatcaattgtaaaaaaaaatggcatcacttcacagattcacgggtctctaagaaataataaaggcttctaattatactttgactgtaagatgtaagattttaattgaagcatcaattagaagatgcatgctgaatccagaattgtgcaactgtgttggagcagggtagggtgtgttttagaattggtgaaatatgataaaatgggcctgggttggtaaactcaatttttggatcttttgaaaggctaactttcaggggaaaggtaagacagaggaagatatcaatgcactagtattaaatagttaagtaggtaaacaatgctggtacaagattgggcccaaaaggtgaacaacagagatacacctgcattgcacactatacctttggcagaatccactggcaactttaatgtctccattggcacacgtgcaaagtcattatggtggttcttctaggttcatagtgattctggggcaaacattttttgcagatataaatagcaaatattagtataatgagaatctacatagagagggaccttctttttttttatgaaagggctttaatagtggtctgtaaaagctatgggggaggcaggcataaaaaagaaaaacaccaaaactgtctgcttggggatatcagagaagtcttgaagaaagaggaacgtgaaacaatgccttgaaggatgagtaagtgtggtctgtggcaggggtgatggaagtaagatagaagtaagggatctcacggagaaagaaaaggaatatgaggcacaaaggagtggaacagcaaggtacatgccgagctcaaagtagtcagaagtggcagatgggctgtgcacaggaggcagctggagaggagtttgtgaaactagacctggctgcatcatggagggcccagcaggctctgcaaggagttctgactttgttctgtggtcagtaaagatgcatggaggattttcatctgagagtggtacaatctgatttgtgtttgaatagttcactctggaagttacatgctggatgcacagctggggagagaaactgaagcagaaagtccttccagcaagaaatgtctttaacaatttttgttggaggaatgcatttgttttaagtgttttccatcttttataatattcccaaagcttgaaggtgcttgcccatcaattcccactcctcaacagctttcttctaattgcctccctaattcctcttgctggaagcattttcactcaccaaatccctagaggtgaaaagaaactatttggatgatacatgtctgtgttgttgcagagcacttcatgcattgcagactgtgagttctttagaccattctcagttgtctatttcacatctggcaatagatacaagtaaaagtagcatgcatcctttaagcactgttgaagtaatatataatgatgctaagacatagtgacagatgggccctggaaacttgggacaataaatcctgttctcatgaaactgtaccagtgcatatcattttgtcactgcaaaatatatccctataaatgtgtttcctttttctgcttgttaaatacttgcatgatatgttgttactttcttaccttatcgttgattggcagtcttcaagtgtttaccgttataaaaattgacatatacagctcttgaagcactcctctcctcagccaccaaagtcctgatttttgcgcatagctcaagcactctctgccctccatttcttgttgatggaacaaacaaattaaaacattttttcatatttaaggtgttaaaatatcttgagtaccactgggcattcatatgtgttagcttctggttgcatattttagaattgggttgcaaatattataacttgagcctcaatacagtagaattttaaaggatctggccatgcttataaatggaatcaaagcaactttgactactatggctagggcttctgacgataatttgataaatgtgccagatacaaaacataatccaagattgtattgcaaagtgtcagatttcaaggtctgcattctacttatttaaaagtattataaacttattgtgccttagcaattattgataatgattaatatgatagtaattttaattcctaccagttcaattgtatttacaatcatggtnaatgtataaaatatgtaaaccgtaanctgtatttgcatggcatgtttnagttctgcnttatgcaaactatgctaatccaggaagtatccataagtcatctcataatctcatctgtgatctttcatggttacaaaggagccagaaaggaataatttctttttagaagcatataaataatttcagtgaatagatggatggatggatgcattcattaagaaatattaactgacatcttgctgtatacattgtatattaactgacatcttgctgtatacactgtatctaagacatggttcatgtacctatctcatcattcatttaatatctgctatatgtcacccactgtactgggtgctgcaaacacaaccnttaaattatccgacttaaagtctcagggtgnttactcatcttcagaatacactcaaacatttaccatgagagatagacttctttttttttttctaatgtaactccagagaatacgttcagagaagatttaagtaggaaactcaaacttcaccatcagatccaataagttggagagaccaaatataaattcatgaaataaccaatatggattacaaaggcatattctgcgagtaacacaagatgcacattatttatatacatgagctgagttgtcagttgttgcatgacgcctgagttaaggtgatcagaggaggctgtagtcagatcactctggaaggaaaggttggcacagacttggcagaatttttcgggaagatggacagcatgaaaaacaatgaacattaaaaagccaaagattttttacaaatggaaaaatgctgagggttgctggcctttgtatcttgggtcataactctgcttataaaagatgtttatgaatattgaatgactcttggaaacatgagccaaaaagtaatacatgaaacttatgcccagaggattatttccatacttcactgggaaaataaaaagaggtcaattctgattgaccactgcataccatctagcatgtgctgagcttgttggtgacacaggaatgccactatatgtttctgaaaagtccaaggcagaagacattccaaacaaaacaattaaattacttcctatggaaaagaaagttttctacattacatggtagaatttttgctaagccagtttttataactgaggtcgttcgaaattaagatgtttgtatgcttctaatttctgatcaccactggtgcttctaaaatgatttaaaataattcattattttatatagtttgaaatgtagtgtgggccagttttacaacattaatttccttagcaaagaaatcagaaacaaaaattcagtcacatctgttattgatagcttttcattctgaattggtgaggccccaatttttaaatttaaatttatttttgccaactggagaagtgattttgttaaagctaaaatgtccaacttaaaaaaaaaattgaaaagctatttttaaaaatttaacatgtaagcattggtccaaaaacatatcagaatacattgtgaaaagcatgtctctcctccacctttggctcctggccattcacttgcccgcccccctccccataacacaaagggaagtatcactgtttatcctcatttgttaacattgtaaatgtacctcattgtaatggtgaactctcattcaatgttccaccgagtttaattcatttctaaagcaaattatgagcaattaaaaagagggtaattgtagtgctgctacatataacattgttaatgacaaaaatgaaataaaatagtaatactgttttgaaaaagtacaatataaattcatcctatttgggtgcccgtgtactcaacgatctataattactcttttcctgcctaatatgtactcattcattgctcccaattttaacatagttctctgctgagtccccataaaatttcagttttcgtttttggaattctttgcctttttaaaaacaagtgacaactctgcctagttcttaaaataaagatgaagtgatacttttctttttttttccctaacacacaagtaaaaaacatttgttcatttaataaaagtccatttgaaaatatacacttttatttttcttcatgacacttgtcaacatataacataatgaatattttatattttatttcattcagaatgtaaattgcttaaagacagaagcgtttgttttgttcaccctcgtatccctagttcttgaacactgcctaggttaaagggatgcttatgagctgagggaaagaaggaagtaagggagggagagagggaggaaggaatgaagggcacaanctttaaataattcatcttctattanccttcttgggtattaatcaaatttaggctcagaaaaagtcaattttatatgggctatgttatctgtatttagattataattaatttacattttcaactgtgcaatatacnaggcaagtatgatgcttatagttaaagatcctctgtgagatctttaacacaactattaaaaaatgcnactataaaaaaagttgaagatgactataaaaaatccttacgttatttgttatatatgtaacctttgtgatgcgtttatgtgtagcacataaacaccagtagcatcggtagcatcctaagatgcagcttttctcttccttgggtataaatcttcatatgatgataatcacacaatttaataaggccattgtccttcttgtcacccagagccaagcttcagagtttgctttcatatctcttttgtgagtcttcccaaacccaggagtcaccaagtttggttgattaccttgcccctgaaaacattatttgtccgttcatttgggaattaacattggtttctctaaactccccatagaattaatcttaccatctttcccaccatgtttatagctttaagttattttgctttatatactagctggatgtgttcacatctgtttttctacaagactgttcttttggttgttggtggtggtgatggtggtgtgtgtgtgtgtgtgtgtgtgtgtgtgttagctaggcattatatatcctctaatgtaaaacaggcacttacccagtgttcattaagttgagtttatacttcatttttgggtcaaataacttccattaagtgttttatcatagtgttagttttgcatacaccataaactatttccagaaacctctcaaggagatatttggatacttatttgttactaaaggtagaggcttatgtcaatatcctgcaacatgttttatcatttgatttgaaatcttgacattttatttctattttcatttccctagcaggtttaatcagaggaggtaatgatatatgtgactgaattcagaattgacttacatggatgattacagaattcatgatttaatgggatagtcctaaaaaagcaaaggcttttctgtttaaagtttctaatttttaacctcatgaagttttctgttttattattccaaaggcattttttaaattcccatgttattaaagcattctaatgtcagaatagataccctattaaaaaatgaaagtggtacatctttatctttcttttacatatgcaatcccaattaaaattaaagtcaatatgtgttttccttgcaaattaagacaagtttcttcttcacttttttttgttagtcttgaattctatttttattacactgtggtccaagagaatggttgttttgatttcagttattttgcattttctgagagatgttttatgtctgattaagtgttgatgttagaatatgtgctatgtgacaatgagaaaaaatgtatatatatatatgtgtatatatacatccacatatatacagtggaaagttctgtagatgtctatcaggtgcatttgatctttgggggtttttgccttaatgatctaatactgtcaaggtgttgaaatctcccaatattatgcagaactgtaagtctctttgaacatctctaagaacttgctttataaatttgggtgttcctgtgtaaagtgcatatatagttaggatagttgggtctttttttattattataaaaaatttgcagaatgtgcagttttgttacacaggtatacacgtgccatggtggtttgctgcaccatcaacctgtcatctacgttaggtgtttctcctaatgctatccctctcctagccccccacctcttgacaggcccgtgtgtgatgttcccctccctgtgtccatatgttctcattgttcgactcccacttatgagtgagaacatgcagtgtttggttttctgttcctgtgttagtttggctgagagtgatggtttccagcttcatccatgtcccttcaagaaacatgaactcatccttttttatggctgcatagtattccatagtatattgggccacattttctttaaccagtttatcattgatgggcatttggattgattccaagtctttgctattgtgaatagtgctgcaataaacatacgtgtgcatgtgtccttgtactagaatgatttgtaatcctttgggtatatatccagtaatgggattgctaggtcaaatggtatttctggttctagatccttgaggaatcgccacactgtctttcacaatggttgaactaatttacactcccaccaacagtgtaaaagtgttcctagttctctacatcctctccagcatctgttgtttcctgactttttaatgatcaccattaaactggcgtgagatggtatctcattgtggttttgatttgcatttctctaatgaccagtgatgatgagctttttttcttcttcactttttaatcagcttgaagtgaatgccttgttagttttcctactaacggtgctaaacctagcagttttaaagaccttcctgttgaccacagtgcactcttggttcttgtaggcagggagctggcttaggacctacatagcatatttggaacaatggcttctggttctttaagtgttagaatttgtgtggctgagaaaaaaaaaatagtgatattttccctttgcatgaattaaatattttacctgcaaagttgcttgtcttttataaattatttttcttataagtaatgctacttaaaatgctaaacttattccttgtgaaagtctgttctattatgatacaaaaattgtagatagcttttattgaaataatatttgacattttcctccacagattgttctttaaaatatttgctttgcctaataatgccatgtaaaatttatttttagggggatagttcctatgtgaaagatcgctggtgtgtttttgatggatttatggtcttttgcctttgggtttctttggtgctacaggtaattaaatattttttaaattacaagcaggtattctccaaaatgggnaattattttgcattaaaangtctaaatagaaantattacanaaatatatatcaaatgtngattgtgtaattgcctacaacattttnattttnnatttttattttaacagatgtgattagtctgtttactctgacatatttgaaatttgaaaaattaattttaattgatacgtaatacttgtatgtatttatgggggactgagtgatatttgatgtatacaaggtgtaatgatcaaatctgcataattagcaattttatatcacctcaaacattttattgttagaaaagtcaatagctgtaatatcttaattattgtcagttatgattgtgaattatttctcaatcagaaacgaaaatcaatactatagaaaattattagctgttaatcagatataaaatatattccctgaaatgaaggaaacaatgtggagcaatatttctgtatttgtaaataaaaaattgaacttaaaaatttaaagtctagtctccccatatttggataggtattagccaacataaaatactatattgcaaatattaaatgctttattataagtgatggggaacgtacattaaaaagacttacaggacagatttgattggaaaataagtagaattaatttctgtgcattcattcagacatgattttgccttttaattcaactaaagttactatattctattgatttgatttgatctgttttgatttggttgtggtaagaatgcctaacatgaaatctactgttttaatgaattttaattgtacaataaattattgttaactgtaggtacaatgttgtacaggtctctagagttcaatcatctattctttatttaaaaattcagaattcaaaacttcacagcccattctgccaactcatcatagtgtttgttgataagctttcctttcatatttttataaagttcacctctcccacaatttaatttcatcactcatatatttttttaatcagctttgtcaggttgaattgtttctcacattaatgtgttcttttccatttaataactattatgtgattgacaaacatttcaaattattattttgcctgttagtaaatagtgcaattcttttttaatattttaaccctttctctattactttattttctagatattcaaaagcctccactaaagaacgttattaacaagaaagacagcatacttactatatattctaagttggcacttcttgtaactaaaaactgtcttaacgaacgagacaggnaaatgaataagattaatgttgttcttattagtttatgtatttgtttctgttaagatagctatagcttcaaaatatactcaaaagaaagtggattttggtggaaaataatgtatggctaaattcagagtagaggtgcacctacttatgctgctaacatctgtatgttccatgactataagcaatgctaagggaaaatccaacctcatttacccatcttttaatttgtaggtgtttgaaattgctgatatagttgatcagatgtcaccttggggcatgttgcggattccacggccactgattatgatccgagcattccggatttatttccgatttgaactgccaaggaccagaattacaaatattttaaagtgagcgctgctttacaataagttaagaggaaaagtttatgaagttttttgtaagttttatgtactttacctacttttgtttctctaggcgatcgggagaacaaatatggagtgtttccatttttctacttttctttctacttctttatggaattttaggagttcagatgtttggaacatttacttatcactgtgttgtaaatgacacaaagccagggtaagtttatctattaactgcattttaatctagttgattaacaaagatatctctgtaattttcataagaataacctctatgtcagattttttaaacagtgcttatgtcctctttgttctgtgtctctgcttcctcggtattgtttttgtccaatttttattttattttattttattttatttttgagatggagtctcgctctgtcatgaggctggggtgcagtggcgcgatctcggttcactgcaacctccgcatcccgggttcaagcgtttctccttcctcagcctgccaagtagctgggactacaggcgtgcaccaccatgcccgactaatttttgttttgtccaatttttaaagacaagtattcaagacaggacagattggaaaatgtaacctggtggaaaagtagattaagcaaatgcaaagtgtacatttagaaaccttagtgtggcaaaatggggagtgtgctaacataataacataaacctagagcttcactgtggaagcttttcatattagaatgagttacatgttcgggaaaaaaagctcatttttattattatttacttttgtcatcattccctaaaatagtttcctaaatgattataacatcatttctagactaaacactattatctgttgcatttgactgttgtagggttggtgggtaagatagaatgtatgtactttaattacctattatgtattatgcaatagagcacctgcctcttctacttggctgatcattgttaatatacaactccagggatgtgatggcaggagagagcagagtgggatgttggcccctggatggtccagaagtgtcccattaggtgttagctagatatagagtttggggttccaagcacaacccagaangtgcaacagatatgactatgccagatttcagggcagcagggattggggataatacaattgattttataccttatattcaaatcttcacataggaatttgtctatttataaatgaagaactcatccataaattaccgattgaaaaatcttgattcaaatcttagaaattagatttaaagcactttgtaaaattcaaaaagagattacaagcagccattcatcttcaaaatatttttaatatatcaatatctacttacaaaccaattgggtatataaggatcattgagtttaaaaacattgaaagggttatgttgtgttttctgcagttggaggatttctttttcataaagtctatatacttgtatatatacaaagtaaataaaccacaaataagtgaatatctctgtgagtatagacatcatgataagcttggctcaattccaacactttattttatgagtttgcataaattatttaggttactcctcaatgaagtagattaataacaggacatactttctagaggcatcaggaagagcaaatgagggaatgaatgtaaagacaagcaaagtaaccagcacatagtaaaggcattcactgtatgccagctgtcattattttgttgtcataaagagatacgtaaacacgcttacatgctgaggtggtaggcataagtgtatcctccaaaatagagataacatggttaaaaaaaaggaaagagcaataaagaagaccaaaatttaaacttgtaagagtgtgaaaggaaaacatctatgagtaactcatggctatgttattttactgacaaagtaactaaattaattctgagtttatttcacatttataatggtncatttaattattagtctctcacagaaataaatttaataattgaatttctaattcatgcacaaacagtatatgtatattatgcaactcctaataattatataatgttctaaaaactaaaagtgaatttagttgtttgctgttgcaccaccccaaagaatgtcaactccaaaaaggtagagattttgtttccacgtgttttcagaccaggaggcatgtgcaagtttctaggaaaatatgaatgtattcagtaacttcaaaacatcagtataatttcactggttaaatgttaaacagtaaagcataatctattagtcaaggccttaattaattttatttttatttagaaggcatgcatctcatacctacttagaaatttaccagagggggctgtgcacagtggctcacacctgtaatctcagcactttaggaggctgaggagggtagataacctgaggtcaggagttccagaccagcctgaccaatacggtgaaaccccctctctactaaaaatatgaaaattagccgggcatggtggcacaggcacctgtaatgccaggaggcacctgtgatcgggaggctgagacaagagaattgcttgaatccagaaggcggaggttgcagtgagctgagatcgcgccactgcactccagcctggaccacagagcaagactccgtctcaaaaaaaaaaaaaaaaaaaangaaagaaatttaccttaggttcacagaagtgtggacagataccatgccttccttgattatccatagtattatgttgaagtaaagatctcattttttcttgttttttaatataacttttatttttgcatgatgtgagtgaataatctattcaaaattatttttaaaagaaaaatactgttgctactaaccccactcctaatcttcattattattgttcaaaatagccttggaaaataactttttgataaccattttctggacatattattattttcccctattattgaaattatgtctctactttaaccactttattgagattttggtgaatataaaaatctttacatatttattatatatgtcttgagggaactatttcctttttctgtcagaattaaatccagagagtaagttattgcatttattattctacttcctgaaaattatgatccttctcaaattaacttgtatatttcttatgaggtacttcctgaaaataatgatcctcctcaaattaatttgtgtatttcttatgaggtacgggtcaagatgtagtcttaacttagtggttggaatggacatcggtatcttaggagaagctctcagtatttccgttaagtgggatgtgagctgtagagttttcaaagtgaggaagttacctttaattcctatagtgccaagagtttttgatcattgatgtatgctaaattttgtccagtgcttttccgttttgttcagttaatgtaatgaattacatgattgactttttaaatagaaaccaatcttgcatttctggtatattgctaatttggtcatattgtattcctctttatacattggaagatttgatttgctaatgtttttgttacaaattattgcatctttgttcacaaggagcattatttttaggttttttaaattgaaatatctttaccgggtcttgaaatccgggtaatgctgtcctcataaaacaagtggaacatgtttccaagggttaatataagattcatattctttctttcttaaatgtttgatagaattcatgagtaaaactcagggcctgaaattttctttgtgagaacgtttttcattacaaattaaatgtattcaacttacagagctatggagttgttatctttcttgttctgttaagttggtaaattttgcttttcaaagagcttacctatttttataatttgtcaatattgcataatgtttatacaaatgccctctgattatcttttgaatgtctgattatctttttaacatctgtagaatagtggtaataagccattttttcctgacattgataatttgtacctttctctccatttttttatgtcttctcaattgtattattcttttcacacacacacaagaatctttgccctatttaatgttctctattgttagtctgtttttcatgttgattttttatcttcattatttccttatttctgctttttagtttttgttttctttttcatctttcataaaatgaaggtatatagacatcttttaacatttttctaatacaataatttaaaaatataattttcctctaagcactgttttagcctctaagcactgttttagctgcatttccaaaatgttgatatgttgtatttttattattattcaattcaaatatcttctaatatatgtgatttctcctttgacctgtaggttatttagaagtgcatttttagaaggatgagattttctaaaaatgttatttgggtgcataattttattctgttgtggtcagacaatagtccctgtgaaatttcagccttttgaaattcattaggaatcattttaaggaccagtatatggtctgaattggtgaaaattccatgagaatttgaaaagaaaaaagtgaaatctgcagtttttgagtataatatctataaatgtcaccaaagtcaagttggctgataatttgttctgggtatttctatccttcttggtttttaaaatcagggttgttctagcaattgctgagagagtactattcaattttctagacatgacagaaattttcaatctctcttatttttgtcagtttttgctttctataattttcaactttaattaggcgcataacgtgtcattgtatctttcagatgggctgacctttttatcgtcatgaattttgaatttttttcctaattagcttcgggatgccagggtatatgatgggtcaagaacatgacgtttcaaatttttcagtaatcataactctaaagtaatgtgtaactctaaaataatttattttatttaatcatcttttatcttattaatcaattgatttgttcccacaattacttacgtgtactttagaccattcctggattgacagtaaaagggagcacatgacaaattcttagttttagagcatgggctgcacaatcctgagcccagcctggtggtgatgaaattaagcccagtactaagagcgtaaatgaagaagaaactcagtagtaagagagtgtggaccaactggtcagatgctctaatcagcaactctcattccactgtcaaaaccttggagattttgtatgttttttaaataggtgaagtgaatcaagtggtgcctaccaaatttattttgtcctctgcatcagtgcgtggcatacaacattaacaatgagaagcaactaactcctataagctattttgggggaatagaggatggtacaaatatcacaactttacagcaaatattacaactccttcaaaacacagtttggcagtttttaaaaaaataaaaataaaagagttgatcaagctgggtacagtggcacacacctgtaatctcagcactttgggagactgaggtgggatgatcacttgagcccaggagtttgagactagagtgagctatgattgcgtcactgcactccagcctgggtgatagagtgagacccagtctctaagaagtaaaaatgaaagaaagaaaaaaagttggacaggcacctgccacatctcatttattccactccaggtatttaaccaaaataaataaaaatgtatgtccatagaaagatttacacatgaatgcccacagcaactttattcatagtactccaaactggtgacaacccaaatttccatcaacagatagataaactaatgatggtatatccatataataaatactgtttaataataataaataatgaactattgatgtatacagcatcttggataaatctcaaaataattatgatgactgaataaagccagatttaaaaagaggacatgttctattattccatttacataaaattctagaaaatgcaaacttatctttaatgatagaaaagaaatcagcagttgcctagtgataaaggttgtggtgggaattaagggattacaaaggggcctcaagaaacctttggggtttattgacatgttcattatcttgactgtgcttctgtacatgtgccaaaacttatcaaactgtacactttcgtatgtgcagtttatgttatgtcagttacacccactgaaactgcttaaaatgtctcagtgtgaagtagaaatctaatttttcatatggttaagcaattgtgcctgcactatttattgtgtgctattacccactgagtttagtttcacctgtctcattcactaattccctgtctatacgtgggtctgttcctgtggggctctgttgtgcttcactggtctattggactacctctttgccaatatcacactctgaaagtctttcagattacaatctggctttacacatggggtcatatttccagctttcagctttacatgggctcaagacctggtcttccgcctcacatgggcattaaaacccaggcattagctcccattcataacactatgtcaactcatgttccttctgttcctactcatttactgctttccttcttatgttttgacattttggaatgcaggttttgtattgtgagcctaaatgcggagtttttaatgctatctatttgtttgtagtggaaggagttgaatttggtgtagtccatcttgccacaaccaggactcacatccatagctatttttttttctatttttgtctaaggtccttgaggacacgtatgagaaaggtaaagaggatattgtgttatattcatctttttatccttactacctagtatcctaacacagaataaataattagtaactatttattgaaggcatggttgagaacatattctatttagacagataatattaacatgatgacaaattgataaagaagatgaaataaaaaagaaacagagaaaagtaagttgctaaaagcaggaaaggagaaatggctaaaaaatagaaaaacaaagtttaagcagaaagaagcagtgaattagttcatatacggagtaagaaaattgaagcaagtggtgagtaagggagttgactgtgtgggtgagggaagagacaaatgcatggaagaaaatctgtaggtagttggatttatttttttagtgtatgtaggctgtagaatatgcctagattgttctatgagtgaatatgaaaatggaaaaagatattgtgtgggttggagacaatcaatatggagatgaagaacagggaaatcttctggataagaaagcagctgtgtagtactgcagtgggtacagaggctggcttcaaattaccgttttattaaaaccaagtcagaatgtgagagttataaaacttgtaaatgcagctagaagatcaggcattatgctggcaaacgaaagctcatattggcacataaatgatcgcagaaattgagaacagatacacgaatgtcttctgaactcatgaccctagagttgcttcagtaagtattctttgctagttccacgctgtatttggtaccaagaaaagaatgatactgaaaaaatgcatgaaaacatgccttgtgatctaaatattagctccagctcattactttattctaaaaatgaagttcaccatgaaaaatttcataaatgttgacagaatgcttagttacctgagacatagccattatggtgaaatggctgcatttttctctttcccatatgtgtaaaacctgcctagtagcatacacacacacacacacacacacacacacacacatttattttgtcttctttttatgtgactctttgtttactatttaagaagaaaatgcttaactagccacatggtagtaatgttgctcataatcacaactgttatagctttgataattcatttcaagaatctatcaataatatcttttttgttgttgtttttttgagacagactctcactctgacacccaggctggagtgcagtggcatgatctcagcttactgaaacatccatctcccaggttcaagagattctcctgcttcagccccccaagtagctgggactacaggcgtgtgccaccacacccgggtaatttttgtatttttagcagagacagggcttcatcatgttggccaggctggtctcgaactcctgacctcaagagatctgcctgcctcagcctcccaaagtgctgggattaaaggcctgagccaccatgtccagccttatttttaaaaaaaaatttcagttcaatattactgcattttaaagccccacacataagcagaaaaactgtcagcaccattttaccgttactattagcccagacataaatgtagttttactcttagagactcaagaaatatccaaactcaaaataaaacagaaaatgttaattgatttgaaaatttttaaacttttctgtttccttttgtagcctctaaactatgactttttttgctgctataaagacacatgcacacgtatgtttattgcagcactattcacaatagcaaagacttggaaccaacccaaatgtccaacaatgatagactggattaagaaaatgtggcacatatacaccatggaatactatgcagccataaaaaatgatgagttcatgtccttggtagggacgtggatgaaattggaaatcatcattctcagtaaactatcgcaagaacaaaaaaccaaacaccgcatattctcactcataggtgggaattgaacaatgagaacacatggatacaggaaggagaacatcacactctggggactgttgtggggtggggggaggggggagggatagctttaggagatatacctaatgctaaatgatgagttaatgggtacagcacaccagcatggcacatgtatacatatgtaactaacctgcacattgtgcacatgtaccctaaaacttaaagtataataataataaaataaaataaaataaaataaaataaatttcaaaaaagtcaaaaaaagaattccctgtgtaagaacattcaaagtgaaaggagcatcatagggaaaagacaaaccgtgaaagcagagaaataggagacgccacatcaagacaccaccatgcttacaagttggcattttattctgaagaccataatgaactgccaaagaagctgaaacctaaaataaaattattgtttttacatataaagatgatttttcacatctatatttatgatctcaccttttaaatcagtatctacttttgaaaatgtacatgtaaccttcaacatatacacagaaaataagataaactaatttatatagttttgtttcaatcctacacaattagagttgatttttactccatatttagatttccagaatcccagctcattaatttaatatttattaagcataaaagctttaagacctagaatgcatgggacgtggaattaagcactagggataaaagaggaagaaaaaaataggtcatgtcctggaggagctcacacttgaggcagccaaagattaattaccttaggcattgattagtaataataataataattagttagaaagtgttatcagatgtcaaaaaagagagaaaatgtgagttttatagtctttgtaaagaatatgacctttgagcaaactctagaattaggaatggagtgtttgaattggaaattaaggcaggataatacctttcagatgaaacaatgtaatagagtactaggatgtataatagaatgctggccccaaactgggatggtagatttaagttgtgagtttctttctacaactgcagagtccattgaataaatacaactttatatattaaaattaaaaaaataaaaaaaaataaaaaaagaaattctgtcatttggaataacaggaatgaatctagaggacattatgctaagtgaaataagccaggcacagaaagatcaatactgcatgatctcacgcatatgtggaatcctaaccagttaattcattgaagtagagaggacaatggtggttatcaggggctgcggcgagaaggagatggagctgcaagggaatgaggagctgctgatcaaagggtacaaagtttcagctaggcagtaagagtaagttttgagatccatgcacaacagggtgacaatggtcaataataatgcgttatatatgtcaacataactaagaaagtaaatttcaaatgtatcatcacaaaaaagttaagtgatatgatggatatattaattggcccgatgtaaccattatgtcttgtatacatacatcaaaacatcacatggtgctgcattaaagtaatacaattatgatttgtcaattaaaatactattatttaaaaaatgtattatacacatctccgagtcacatattaagctaaggtatgatggctgtgttttcagcaatcagtgttaccagctactgccgtgacctcatttctcttaatcatttaagagaaacgattaagagaagttgttaacaattgttccaagttctgcctaattgatttgcatgtattatagcctaatcatgacagagatttcagaccagaaacttgcatatcttgggtttcatagttgacctccagaactgattgtgtttaatatgtgaggcaaacaagataaagaagcaacacaatatatacagttttgcttttaaatggaacttagttcctgtaaggcccaatagcaggaaatcttagttatgtcttcagtagcagtactagtaaagctttagattcttctctgttaatgaatggagacctaaggcaattatgaaaagagtaatgagatttgactaagcaaagcagctcatttcctaaagaaagttgaggagagcaatggcttgtagcagaaacaggtgttgtgaaataccaactgtgatggcagaaatctccccgaagctggaacaggaatgtttaaaaatgagctggcaggttaaatagaaaagatgaagacttacctgacaccttattatcattcctgaaaaagaagggaaacatattcatagctcggtatcaaataatgaaatcttatttccaagagtgagataattagaaaaactttaaggtaagtaagtgtaaccattaaatatatgaaagtttaatatagagagagcaggattcaaacaggaactcaggaacatattcgcgatggctggaccactagctttaagcccagaggttgggtgatcacacatcacagatgcccaggacaatcttggcttatccctactgtttcaaagtaatcactaacagcctgttattcgattttaaggaatctcaatgtagacaatgcattttatggtcaccttgcccaggggaagtttatagaacttggtattttataaaatatcaccaagagttgaattagagataatataaagttggatttttttgcagtttgctcttgctggtccaaatacattttacatttattaaaataaatacattaaaatttatcaactaaattaaaatttatttgtcacgtattctttagggtacttggcaaataagtgaagtattgtcttataaattttgttgacaaaataaagaggttttttcatggaatgctatgcagccatgagaaagaatgagactatgtcctttgtagcaacatgaatgaagctggaggccgttatcctaagcaaactaacacaggaagagaaaaccaaataccgcttgttctcacttatgaatgagagctaaacactgagtacatacagacacaaagaaggcaacaacagacactggagcctacctgagggtggaggatgagaggaaggtgaggactgaaaaactacctatcgggtgctatgcttattagctgagtggcgaaacaatctgtacgccacgctcgcatgacacgcagtttacctatataacaaacctgcacatgtatccctgaatctaaaataaaagttaaaagaaaaagaaatacaaatggaaacatactggaatttagagcattaatttttaatttctaaggactttatcaagagacagttttgtagaatatgtaaaatcataattgccccaaattcatgcaacggtctatgacgtcttcttgattcctatgtatttatttatttacatatgtttattgacacaatagatgtnaagtatttttggagtacatatgatatttcgatacattgatgtaaggcataataatcaaatcagggcaattggcatatccatcacctgnaaacatttttcttaatactgggaacatttgaattattctctactagctattttgaaatatgtaataaattactgtttactatagtcaccctactgatttatcaaacgcttaggtcttatttattttatctgactgtatttttgtacccattcaccaacctttcttcatccaaccctggcctttacccttccccttctctggtaacaaccaatctactttctatcttcataagatccacttacttaaggcctaaatatgagtgagatcatgtgatatttgtctttctgtgcttggcttatttcttttaacataatgacctccagttctgtccatgcagctgcaaacgtcaggatttcactcatttttatggctgaatagtattaacactgtgtgtgtgtatatatacatatcacattttctttatccatttatccattggtgggcagttaggttgattccgtatttggctatttggaatagtgctgcagtcaacatgtgagtgctgctatctcttaaatatatagatttcctcccttttggatatatacccaatagtgaaattgctggatcatagagttgttccatgtttagtttttttcagtccgtactgtttttcacagtggctgtcagaatttgcattaccaccaacagtgtatgagtgttcctgtttctctacattctcaccagcactcactgtcttttttataaaagccattttaacttggggtgacatgatatttcactgtagcttttatttgcatttccctgatgattagtgatgttaaccatttttatatacctacttgccatttgtatgtcttcttttgagaaatgtctattcagattttttgcccatttcttaatcagattattagatttttcccattgagttatttgagttctttatatattctggttattaatcctttgtcagttgaatcatttgcagacattttcccttattctgtgggctgtcacttcaatttgttgattgtttcctttgctgtgccgaagttttgagcttaatgcaattccatttatctatttttgctttagttgcctgtgcttttcagattttgcccccccaaaaatttgcccagtccagtgtcctggagcatttacccaatgttttcttctaggagtttcatagattcagttcttagatttaagtctttaatcctttattatttgatttttatatgatgagaggtaggagcataattttattcttctgcacatggatatccatttttcccagcaccatttgttgaagagactgtcctttccttaatgttcttgatacctttgtcaagaatgaattggctgtaaaagcatgtatttatttctgggttttctattccattccattgggatattttctgtttttttaccagtaccatgctggtttggttgctatagctttgtagcatattttgaagtcagttagtgtgatgcctccagctttgttctttttgcacgagattgctttggctattcagaatcttttgtgttttaatataaattttaggatagttttttatattcctgtgaagaatgtcattggtattttgatagagattgcattgaaactgtagattgcttcgggtagtgttgacattttaacaatattgattcttccaattcatgagcatcgaatatctttccattttttgtgtccacttcaatttctttcatcagagtgttatagttttcattgtagagatctttcacttctttagtcacaattattcctaggcatttttaattttttaataaatattgtaaatggcattgctttcttgatttatttttcagattgttcactgttggtgtatataaatgctattaatttttgtatgttggttttgtatcctgcaatttactgagtttgcttatcatttctaacagtttttggtggagtctttaggattttctaagtgtaagatcatgtcgtctgcaaatgaggataattttatttcttccttcccaatttggatgtcgtttatttctttctcttatctgattgctctggctagaacttccagtagtatgttgaataaatctgatgaaaatgggtgtcctgatcttgttccagatcttagcagaaaggctttcaatttttccgcctttggtatgatgttangccatgggtttgtcaaatatggtctttattgttttgaggtataccccttttatactcaattttttagtgttttgataataaagacatgttgaattttaccaaatgctttttctgcatctattgaaataataacatggtttttgttcctggtttatttatgtaatgtatcatgtttattgatttccatatgttaagccatcctttcatttctgggaggttaaataatatgtccagatggtaagtaagaaagccaggattttaactcaagtctaacttccaagcctatgtacgttttaccatttgagaaacttctgtattttaattctacattcataaacctatagacatatagagatccacatnccatatgtataaactgtttnataatatatgtagtataatttnctnacattaaagtttttnattaangtctggtttctantccctatgtatatgnttatgtttcatgggattcgacttncatctaaatnctataaactagntgatatattnatatatttttagtactatctatatatctcttttnaaaattgttctgcacattgatctccaagatgtgaaccacctgaggtgccagaaaccagcctttcatatgataataatattaataaagtaaatataagaatatatttcagaataaattgaactttcagatgaaaattaataccagcatgatttttaccagaatagcaaaaatgtttgtgagagtcttcagtataccaaaagaaaaccatgcagtttgaatgggggtctaaaaggaaangtaacttattttgatccatcttttgatcatatgtttacttcccaaaaagaagataatgtaatattataggttataagattcttatttttagtggtaaataacttttattgaacaattgctgtatgttattttctcacttaatacttataacaacccgataagttatgttccatttttacactgtatattacagccaaggaaattgaagctgagtaaagttaaacaaagtggctgagatgaaacagcttgtatcagaagcaagactagacccatttctccctcacactgctttctatgctttcagttgtaacttgttcagtgtatgagataaccttccttcaacatgtgtgtccaaaatgtttgaataaaatatttagttttcctttagttacaaggagcctattcttaaaagacaagttctcagtgggacgcatgagtaagtggttaattacgattgcttattttaagactgaattctaaaggcatgcactggaacttctgaacatcctctgtgagtgtggaatgaggagctctacctattctgtccctcacaaatagtgtttactgtagccgcctccacagaatcgatatttactatcacttttgtatctcaggttccacccaaccagcttggttttccccatgatagaaaccataataagtatgcataactacagcagcaccaagagtaaattttngaaaattaatttttaggatgaaatctctttggtaaagaaaatcacctggcaaaagttgtaaaaaatactgattccaagccacatttgtgattcgtatttttaggcaaggctgagattctcagaattggccaggttagaaatcaatcgtttaaaattttagttttgcaattaaactccaaaagatatgttatagagtagaacacaaaattacatggctttttaaaaaaatagaccttgagtcttcaaagaaatgttagtcttctagctagctgcttactgccctgcccaagggtagcatgcctgtcctcttcccttaagactatttccccagaaatgtccaaggagcaccagaggtaattagagcctcctgtctttcctgcaaagaggcaggtatgtctttgttttctgggacggacaaccatctcaattttatttatttagtgactcttacatgttcttatttctagagattgaattgggctggtgtactttgtggttgagtgtggttggagtcgtcaggggcattggcacttttaaaatccctcactgactttacttttcgtgttcacatcattggggagtttgtaaatccattctttcattgctataaagaaatacctgaaactggcagggcacggtggctcatgcctgtaatcccagcactttgagaggtcaagacgggcagatgacttgagactaggagttcaagaccaacctgggcaatatggtggaaccccatctctactgaaaatacaagaaatagctgggaatggtactgtgcacctgtgatcccagctacttgggaggctgaggcacaagaatcgttggagccgggaggtgagggaggttgcagtgagccgagatcatgccactgcattctagcctgggtcacagagtgaaactccatcgaaggaaggaaggaaggaaggagagagagagagagagagagagagagagagagaggaagagaggaagagaggaagaaagagaaagaaangaaagaaaaaaagagagaaagaaagaaaaaagaaaggaaangaaaaagagaaagagaaagaaaagaaagaaagagaaagaaagaaagaaaaagaaagagaaaaagaaaaactgagactgggtatcttatttcttttattacatttttgggtcctatatttgtatcatacagaatgagttctgtatatttttcagatggaaaagaaatattgacttaaaaggtacctctcaaatcttaatgtgtacaataaactggagatcgtgttaatgtagattattttttcagaagatctggaagaaagcctcagtttctgaatttttaacaagctcaccagtaatgtcaatgtttttggccaaagaagaatattttgtcaaaaattcagtaagtggaagagcctgtgtttctttttctagtttttctggcttgtaaacaaagatgagagttttcatttaccaaagacagatatatgcaaagaaaacctaagaaagaagggcagctgccagattaaatgtgatggtttatgcacatcagctgcataaagatacctaataattaagagaaaaataattaactctgtagtgcgacagtctgtcagagagctctttaaccaagtgaatcataaacattaactgcactaggacaaatttttatgantgtgctgatgcacacagaaggacgcagcatcactgctgtgatattgccccctcaaaagtacattatagtctaaatttaaccataaagagacatcagttttatgcgaagctcaagatacacgtatctttcatgttctgtaatttgtaatgttgattttaagtagtctttagcaccctagggagcaagcatctcctaataatttttttcagaactttctgggtaataaatgccatcctgttaaataagtattttctactttttttcctgctttgtatccatttattcacttgtttgctttttaaaaaatttttactttaagttgtgggatacatgggcagaatgtgcaggtttgctacctaggtatacatgtgccatggtggtttgctgcacctgtcaacccatcatctatgnttttcagccccgcatgcattagctatttgtcctaatgctctccctccccctgcccctcaccccgcgacagaccccagtatgtgttgtatgttctcattgttcaactcccacttatgagtgagaacatgcgctgtttggttttctgatcctgtgttacgtttgctgaaggtgatggcttccagcttcatccatgtccctgcaaaggatatgatctcatttctttttatggctgcatagtattccatggtgtatatgtcccacattttcttagtccagtctatccttgatgggtatttgggtggttccgtgtctttgctattgtaaatagtgttgcaagaaacatacgtgtacatgtatctttatagtagaatgatgtctattcttttgggtgtatacccagtaatgggattgctgtgtcaaattttatttctggttctagatccttgatgaatcatcacattgtcttccacagtggttgaactaatttacattcctaccaacagtgtaaaagcgttcctatttctccacatcctcgccagcatttattgtttcttgactttttaataatcgccgttctgactggtgtgagatggtactgcattgtggttttggtttgcatttccctagtgatcagtggtgttgagctttttttcatgttttttggctgcataaatatcttcttttgagaagagtctgttcatatcctttgttcactttttgatggggttgtttctttttttcttgcaaatttgtttacatttcttataggttgtggatattagacctttgtcagatgggtagattgcaaaaattttctcctattctattctgtaggttgcctgttcactctgatgatagtttcttttgctgtgcataagctctttagtacttctagtttaattagatcccatttgtctgttttggcttttcttgccattgcttttagcattttcatcatgaaatcactgtccatgtctatgtcctgaatagtattgcctaagttttcttctagggtttttatggttatgggtttaacatttaagtctttaatccatctcgatttaatttttgtctaaggtataaggggtccagttttagttttctgcacatggctagcaagttttctcagcaccatttattaaatagggaatcctttccccactgcttgtttttgtcaggtttgtcaaagatcagatggttgtagatgcgtgatgtgatttctgaggtctctgttctgttccattggtctatatggctgttttggtaccagtgccatgctgttttggttactctagccttgtagtatagtttgaagtcaggtagcatgatgcctccagctttgttctttttcctaaggattgtcttggttatacaggctcttttttggttccatatgaaatttnagggtagtttttttctaanttctgtgaagaatgtcaatggtagtttgatgggaatagcattgaatctataatttattttgggcagtatggccattttcatgatattgattcttcctatccatgaggatggaatgtttttccatttgtttgtgtcgtctcttatttccttgagcagtgatttgtagttctccttgaagaggtccttcacgtcctttgtaagttgtattcctaagtattttattctctttgtagcaattgtgaatgggagttcattcatgatttggctctctgcttgtgtatttttggtgtataggaatacttgtgatttttgcacatccattttgtatcctgagatttgctgaagttgcttatcaggttaaggagttttcgggctgagatgatggggttttctaactatagaatcacgacatctggaaacagaaaatttcacttcctctcttcctatttaaataccctttatttctttctcttgactgattgctctggccagaacatccaatactatgttgaataggagtggtgagagagggcatccttgtcttgtgccagttttcaaaaagaatgcttccagctttttcccattcagtatgatactggctgtgggtttgtcacaaatagcacttattattttgagatatgttccatcaatacctagtttattgaaagcttttaacatgaagagttgttgaattttatcaaaggccttttctgcatctattgagaatcatgtggcttttgtctctggttctgtttatgtgatgaattacgtttattgatttgtgtatgctgaaccagccttacacttcagggatgaagccgacttgattgtggtgggtaagctttttgatgtgctgttggattcggtttgccagtattttattgaggatttttgcattgatgttcatcagggatattggcctgaagttttatttttgttgttgttgtgtctctgccaggttttcgtatcgggatgatgctgacctcataaaatgagttaggaaggagtccctccttttcaattcttttggaatagtttcagaaggaatgataccagctcctctttgtacctctggtagaatttggctgtgaatctgtctggtcctgggctttttttggttggtaggctattaattactacctcaatttcagaacttgttattggtttatttagggagccaacttcttgggagggtgcgtgtgtccaagaatttatccatttcttctagattttctagtttatttaggtagacatgtttatagtattctgatggtattttgtatttctgtggggtcagtggtgatatccccgttatcattttttattgtgtctatttgattcttctctcttttcttctttatcagtttagctagcagtctgtctatctattttgttaattttttcaaaaactagctcctgaattcattaattttttgaatggcttcttgtgtcactgtctccttcaattctgctctgatcttagttatttcttgtcttctgctagcttttagatttttttgctcttgcttctctagttcttttcattgtgatgttagggtatccatttgagatatttctagctttctgatgtgggcatttagtactataaatttccttcttaacactgctttagttcccagatattctggtacgttgtctctttgttctcattggtttcaaagaacttcttgatttgtgccttaacttcattatttactcaggagtcattcagcagcaggttgttcaatttccttgtaattgtgttgttttgagtgagtttcttaatcctgagttctaatttgattgcactgtggtctgagagactgtttgttatgagttcagttcttttgcatttgctgagcagtgttttacttccaattatgtggttgattttagaataagtgccatgtcgcactgagaagaatgtatattctgttgatttggggttgagagttctatagatgtccattaggtccacttgatccagagctgagttcaagtcctgaatatccttgttaattttctgtctcgttaatctgtctaatattgacagtggggtgttaaattctcccactattattttgtgggagtctaagtctctttgtggatctctaagaacttgttttatgaatctgggtgctcctgtattgggtgcatatatatttaggatagttagctcttcttgttgaattgatccctttacctttaagtaatgccattctttgtctttttttatctttgtttgtttaaagtccgttttctcagaggctaggattgcaacccctgattttttttttttctttccatttgcttggtaggtcttcctccatccctttattttgagcctatgtgtgtctttgcacatgagattggtcccctgaatacagcataccaatggatcttaactctttatccaagttggagactgggtattttataaagaagaaaggtttaattggctcatggttctccaggctgtacaggaagcatggcaccaggcacctgctcagcttctgatgaggcctcaggaagcttacaatcatggcagaaggcagacggggagcaggcacatcacgtgacaagaatgaacacgagagagagatagtggaggggaggtgccctacacttgtaaatgaccagatcccctgtgaactcagagcaggagctcattgatcacctaggagttggctcaagccattcatgagggttctgcccctgtgatccaatcacctcccaccaggccccacctccagcgctgaggagtatacttcgatgagatttgcgcaaggacaaatatccaaactatatcagggagtcgttagtatgtctggccaccttgaaaggatgaaagcttattggagtggaaaaaaaattgcctctctccagccatacaactcagtcaaaccacattcaattaacataaatatattttgacctccaagtatgtaagaggtgatttatattcacaatttctaatttataatacagtttcataaatttgatcttcacaagaaagatgagcaaattgctccttgccaaagttaattggctgaattaccaactgaagttctgttatgcttggccccaaattctggtggcccctttccattatgtcaactggctctctttacatatagattctcaccctgtagtttccagttcctaaggcaaaaggagaaaagttgctgcgtgaggatctcttggcccagattttaaaagtcaaagacagtggactgccttataacctcaaacggtttcacatttcagtttggagctgatcagtgcatcctctgtgtcctgatgcttaacttttcttttcttttctttttgaggtggaatctcactctgttgcccaggctgtagttcagtggcatgatgttggctcagtgcaacctctacccgctgggttcaagtgattctcctgcctcagcctcccaagtagctgggattacaggcatacgctaccaaacctggctaatttttgtatttttagtagagatgggttttactgtgttggccaggctggtctcgaattcctgaccttaggtgatccgcccgcctcggcctcccaaagtgctgggattgcaggcgtgagccactgcacccgaccaacttttctgatattattttgaatatgttcatgccctctcattgccttatcctaacccaactgcagtcttctggcttctgtcaggtgcactgtcccttatccatactggcaaagtagttgagggagtcaacttggggaacatatattattttaaaaagctattcagatataacaagataactatttaaaatattcctttaaaataaaatcatgtactgcattgtcacttaaatttcatagtcacaatatttgaaaaaatacttaattttaccatttttatgcatgatggctacaattctttacaactttgcctaccaaccctttcaggaatgggtgaaaggagtgggaaaatcatattaaaatactcagttttacaacttatttcctagaaaaaatgtgccaggttaacttgtttattaaaagaggtgtgatgtcaatattatttcctgcttcagaatatttatcttgttttgcagttccactaaaagtccaggcggagccattgaaagttttccaaaatcaataaataatttatctctgcaaataagactcttgctttccagggtgggattagtattccaaagcatccagcattagaattagctagaccaattcataggagtttataattaatcctgaccagaggaagctaaataagagtgaaaatgaagtcagatgattgtctgaagtgaagctggaaggcggtaacagccagactaagttttctgtaagtacatgcaatgaccaggaaaacctctcaatctaggggtcagcttttagcaagataatgtgtcttgctaaatttttatatctgccacaaaaggcaagacaagcaccacgtaaggtatcaaaggctatattcattattctcttgagtgcaaaagagtctgtgtctgtgtctatgtatgattgtgtgtgggtgtaattttctccatgggtttatttagaattgtgttcctctatttttgatgtatttctagaacatactatataaataaaaatgaagccttacagatgcaatcaaaaacaaaatgctcaagttgtatagtgttatctgttgcatgtggatttgttgggctacttatttatttacatcactgacaatttcttatggaatgccgacttcagaaatgtattgtagcttggactctgcaaatgtcttttacaacaaatgggtcaaatttcgataccaagaagtcatccttagcctgtttgtctttagctttttaacaaaaaaaggtaataacaatgtaaacaaaattgctatgtataaattcctatcataggtatatgttttgaaaatctagcaagtaaatttagtaataatttattttgtgagtatcttttgaccttttttgtcctttctcacctactaggaacagtttataatcagctcatgctgcatcagtcacagtatccatttagggtagcacgaatccttgtcttttaccctaatctataaatctctaacagtgtagtttaaaaattgcttttgtatgataacaactgcttgttaacaatccttgtatctcactaaacagctacactcaccagagaaaaggtgaaaaggtctctctctttctctctctctttctttctgtcactctctctctctcatatgcaaatgcaggctctgacattttcactttccagaatgttatgctgtttattccatccctagaactagtacgtgtgcatcctttaataatagtcactccaagcaacaccctattgcaaggttttcccagtacccaccatctgccacccgtagtcatagttgatgtttccgctttgaacttcagttaacatcaggagcatatagatagctcttaaagcattcatcacactctcctgtggtcattccttttttattctctctcatactaagcagtaaagtccctgagtccattttattccttttattcatctccattcctagcatgctgtgtggcatagagtaggcttgtcaaaaataattattatgaattaataaacagacattcaaatacaattaagtgcccccctacgaatacaggaaaatttttctgaaaagaggctaagtcatcataagcatagtcatattcattaatgacagtgattacagaaggccaacactaagtgcagtgccagaagccatgatggtagttccatatctgtagagaaagacgatattgtgaataacgaagagaggaatcttaacatgaagtcttaagctattgcctttgtcgttggtgagtcttgtggtgagaggagaactatgcacagtgaagcatatgttgaattatggggcacatgcttttgcttaagtatatcccttttcttttatatgaaagcgttatgaggtccttctgggaattgttttttaaacagctggaaagtttctcatttttcagatgaggaaactgaagctcaaatgtttcaagtgaatctcccaaggtaattaagttggttaatcaccgaactgagatgatcatctttgtgttgtgatatacagctcagtgctctgcaccgctaggagtggtgaataacttaaaaccacaggactctgaggtcctgaggtattgtttgtggccattatcaacaattttagtaacaagttcagaacacattttcttagatctcaaactcctacaaaatgttatttgtaaatagaatgttttgttaagaattaaaaacaaacaagcaaacatatgctaggaggataattgaaggcttttaaaaaaacaattagctgctcccagggattctagtactcattttgatacatggaagctactaaagccagaaatttaaaacattgaaaatcagtgccaatttgtggattattacacttaaaagtgcataaaacaacaggatccacttattaaagagtttctgtagtttacaaataaataattcaccatagatttaaataatggtagaaagacacattcattcattcattcatttattcagaaaataattattaaacatctactatatattaggtttctccagagagaatatagaatagaatatattatatatatgtatgtgtatatacatatatgtatgttggcttagctcaaaagatatgtctatagatacagatacagatataaagagatttactttaaggaattggctcatgtaattttgtaggctgttaagaccaaaatcttcagggcagcctggaaatttcagcagaagctgatgtggcaggccaaaggctctgccaaagatgtggtaggccagtgacctctccctgcagagaagtcaaaatctttcctattaataagatgagtcccactcacattatggagggtaacatgctttactcaaagtctatcaatttaagtgttaatcacaactgtaaaataccttcacagcaacatctagactggtgttcaaccaaacagccaggcaccatactttagtcaagttgatacataaatttaactatcatagacgctgtgtcagggacttcatggaattggaaattacactaaggaacagttgagtttggaagacatccaatgaacagatgaccacacaaacatgaattgcctttgtctcaagtgccagaaacaaaaagtgagggtaccttgagaagttatagcagggaaacctggcccagtggaaggcacttggagtggctcccccgagaaaatgatagttaagctggggcctgaaggctacaaaaatggggttaaccaaagaaaacaggagagacttccaagtggaggagaagaccctgcattaggcaggagctgggagaaggccaacgtggtcagacactgggatacttgggggaaagggggtacaaaggggtgtttgaagtgtgggaggagccagatccttcagtatcttgcagaccatgttacaggtttgccactgttaaggtaggacagtgattatgaagggtctctccatatgctttgtgccaaatgtatcagaaaggctaagagtgggaggcagggtggccagtgaggaggccagataagagtgtgtgtagagagggagatggagagaatggaattagctcaaaagatattttgagaatatgttggagaacgtgacgattacttatgagggttgagggagaagtaggtatcaagcatgattctaagacttcaagtttgctccacggtttggaagctggtcctgtttgttatgacagggaactctggtttaggatttcaaattgaataagggacctattgagctggaggtacttgtggtgtagccaagtggagaaatctaataagcagttggacatgtagctctaagactctacaacgatgtaaaggatgacagagaattgagaaatcatgaaagtggatgagattgcctgggagagttatcatgaaaagagaaaggtcaaagacagagctcagagcagctcatgactaaaggtcagctcacgactagaggagatggaaaagcagaatggcctnaagaaatatggacnaaaccagcaccttcaaagccagcagagttgcatctgtgtcttcctcccatactcacatctctctccanaccacagccatgatcactgttagtgattcatggagttaggttgggtccacctggataattcaggatcatctcccaaatttaatgtctttagccttaatcacacctataactttagttcccttttgctatgcaacattatctactcacaggttccatatgttagaatggggacatctttaggggaccagttttttctctaccacaaaaacaacaattcagagcactgctgagaggtcacagaagataaaactaaaatttttctgattgggtatatcatatggaagtgaacaaaagtaatttcattgtcttggagtcacagaaatgggctaaaaagttaaaagagggagattacaagtagatactttattcaagaaatmtgtgagtaagaaatgctggtcttgaaacccgtttaaacatgagtggatatttattaccactgaaagaatcattgtctatgtccaggattttcctggtggggcaagaattccatccaccccacccccaactccagagagtcacttgttctctatccaaagtacctgagggaaaatgactacggaaataattaaggttaatgaataaaaataaactctggaagattcttgttaactgaaaaaaatgaaacaaagattaacaaatatatattctgaaactcttgacattcatttcaatttttaaaaacagtaattaccttgtggttttagatacatgtgttccagggcaattaatcttctaataaatgtgtttctaataaacatttgctaataacttgatgttggtaaaaatgtttcaagcctgctgcctctgaagccaaagacttcaaaataatatctagtgagagttgtgcttaaaatgagtatcagcactaatatgaaagaaaaagcaaatataataggaagaaaaaaggaaaaaatactctaatatttataaattaatttaaaaaatcataatcattaacagttgagtgattaacttatttttggcaccattctgagtgttgcacattatctaatttaatcattaaaatgcccctacaaagaatgcaataactatcctcattttggnaagtgggataactgaggctatacacacagggctggagtttcaatctaggaaatctacaactttcaaactgtcaataacagtaggtaacatctagcattgattgtctatcaagtacctgttttatacaatttacagatgtaaatacttacgtctcaccccaggcttatgaggtagacgttttcacaccattatggattggaaaactaaagcatgcttaagtcacacatgtaataagtaggtcagaactcacagccaagaagattggctccaaagtccaatttcttctccaaatatctgaaatatgatttttttggaaactttttcatgtagacagagtgttatcttagacatttctgctattgtatttttaaaagtatttggaattaaaaatatatgtaattttgatgggctagtagacccttccctctctgaacttcaccttgccttcaagagtaaatgaagaactaggtgatccatatagtatctcttagctgcatagttctatgaacatgctaaagtttcttctcctcttttaacaagtctttattgttctgtttttacatgctgggatttatgctacattgtattattgtaatgattaataatcatttagatcaagcattatttgagtacagtacttgcaacacattaggtgctatgcaaagtgcattacttggtttactttacttaaccctcacatgagctttgcttttaattatgattactctctgtaccttacagatgaggaagctgagcttagaggcccttagagccttatttaagtcacacaaccagtaaatggcagagctgggatcaaacccagaaagcctcactctgaaactcagaagcccagtctccttaccatcttactgttatattgtaggtaccaaatcattgatgacaagcatcgttgatgaggttaatagtatttgtaccacacattctgaaacagagtcagatcagactgaagcaaagatgaatcatttggcctacatgtaagaatgagaaatggggagatatacacgatgtgtctgtctaaaataatatctttctgacccacccaattgtggtaatttccacaattagagcctttcccttgggttttacagatggggtccaaaggaccagcttaaaatatgactagagggagcaatgatttaaccacttatttcctgagaccagcagacagatgagctcatctgctgctgacaaagggcagaaccctaaaggaatagcttagggtgtttgaatcatgggtgaagtttctggagctttgtttatggctgagaagtgggcaaagtatcaccagccatggggtgtgtgtgtgtgtgtgtgtgtgtgtgtgtgtgtaaaactatgtacgtgtgtgtatgcgtggtggcagcaaatagctaagggattgcacaattaggtgtcagactttagagaaagcatattataaaagggatggaagcagcaactgaacataagaaggtattaaagaaaaagagtcatctggtaagtctacctcaatttggtcagaaactcacgtgactgaggctgtcttagggttgttctccatttaagaaattcctagaaacattgatgtagtgggaaattagtgtaacacaattatggatttagaaggcttctgaaatcatcaagcccagactttggcgtctgctcaatctgtgtgggatacctcagatagcagctatttggggtccatttttaaggtcaacccagaatgagagtctacagtcagcagtcctatgttatctatacttgctgtcaggaaagccattacatatctaaatcctttatatcaagttgatatccaacctattcttaagaaggataaaaatgggttatgtcattatccacctaaaggattcttatattcctagagctatcactaaatcacctcataatctctctttcctatgggataccttgagttccttccatttctccttatttgtctggttatttctaaactagacaccttccaacactcagaaggacaagtatcattgatgagtttcgtagtattcgtactgcaaagaaaatgattttcatcatgcaacattgcttagaagtggggaagtaaaatgtgtgcccacagtgagacaagaattttgtgaatcacaatgactcctcagacaagttaacgttctttgcaaatctgtagaaaaattccttcctttgcttatctgggtctcagtctgtaatggcaaaaaccgtaatggtgatagtattcttataaagagctattgtggggatgaatcagatatcatttgagaatgtttaatatgtaattaatgttatgaatatcatttatggattatgaatcttaaaactatattcagatacacatacacaattttgcaaataacatcatatatatatatatatatatatatagagagagagagagagagagagagagagagagagagagagagagagagaaagtctctcaaggtctcatatatatattaccaattaccacagttgggtgggccagaaagatatgattttagacagacacgtcacgtatatctcctcatttctcattcttatatgtaagccaaatgatccatcttcgcttcagtcttaactgactctatatttcaaagtgtgtggtacaaatattattaacctcatcaacaatgcttgtcatcagtgatttggtatctacaacataagagcaagaaggtctctcaaggtctctctttctctctctctccctctctaaatatatatatatatatatatatatattctctttgctgtactcttagatagatagatagatagatagatagatagatagatagaaagagcaaaggtctctcaaggtcttgctctgttgcccaggctagaatgcagtggcatgatcatagttcactgcaacctcgaacccctgggctcaagggattctcctgcctatgcctcccaaagtgcaacgattacaggcatgagcaacctcacttggccagatacatagatattaatgaagggtaattacattattcattgtaatcaacatttatgattgttgaaattgtctaacactttcatgtgttgttcacaggaatgtaacctggaatagtttagctattccagacacacactgctcaccagagctagaagaaggctaccagtgcccacctggatttaaatgcatggaccttgaagatctgggacttagcaggcaagagctgggctacagtggctttaatgagataggtctgtcttactggtaaaatacgttttaagtttctattttgtgatgtttatgaagtggacagaataattaaataattgatactaatattttgactggctgtagtatagaagaaatacaaatataaattttaattctatgcctattttataatatattctacccgacagtgcgtagcatacaagctgtgtatgtatatgggcagcagtgtttgtatggagttacagatctctcatttttgactatttcgattctcaataacrgcatttaagcatttaaaaatatttgtttgttgaacagggttctgggttgttgactcaaagataaacaaaatttggtttcctttcatatgtaactatgtaactataccctaataggcaaggtaagtcttaaaaactgtaatactaggtattgatgagtgcattagagctgaaaagtgctccttgattagaatacaattaatgaactacttagtgaatatgcatcaactaagtaataaatgtatgggaaatgcttgtgcatgagaactatgtgctcaaatgtgtacattcttcttggcttgccttattaagtcctaaaatgtgccattacatacataatgaaagaatttagcatctgccatttagttgatatcattgtatttttataattccatgcaagttgcgcatattatattattaaagcaaaaaatgtataattccaaacaagtattttatgccttgagaattgttatcagagtaattttttaaactaaaatttcttcaagtgtcaggcctgcgtgctaaaccataaagttacagcttttatatagaatatagtactgaacgtgggctgatggtatctttttaaagtcagttttgcaaataacatctcaggttttcatctttatgttttagtatttcctcatattttatttcaagcattatagttagatggcattcttccatctcttttgaccagtaatattgtctcctatttcacaaaaatccagaagccaactgatgtgaagcaatttcaattttctatactctcttaaacttatccaactttgtaattagtcttaaatccttccctctgtctcaaaagtacagaggcatcatgaattataccaataacttttaggcatgaggaagaaatcacagatgtggtatgggaggcgagatgtatataggctgataggctgtgcggaataagcttcaggagttccaacagctgggcatgactagaatgcatgccttatcagagtctgcagctctttcttttagggtctgagtgtatccatgtcttctatggagagtggtcaccattccagggttaaggttgcccttacagaaaataacctccaattttttaaaatcatgtagctaaagcacctcataccctcaccgtttttaatgcaacaattgtacccaaattcaaaatgcagcattatattcaagttgattcatctttcttcactctgacttacaatggctttgcttaatatagggtagaatctctttttgagagaaaaagtctccttgtactgtgctgacttgagaggaccagttctaggaggaaattgccttggaggcagtcgccccctactaaagcaattatcttactatagctgtactgttaatttatctcccaaggatttaagctccttgaaaacaggaagtgcatcttttcgtcttcaagtctccagggcacagggctatgcctggctcacagggagccctccataaatatttactaataggctcaacaaagcgtatgtctttttaattcattggtaaccaggcatgtttgtatgaggaggcagagccacaatagagaatttagttgttttcttgttgttcttttgcctgattttgtctgtttttgttggcaatattcattattcctcatgactccttcctttctatacaggcttaatttcatccgtttgcttatttttcccaaattcatcatcactgtatgactaattgttagataatgaaatgttgatcttgtaccataaccaaaacatttttaaatggtcgttataatttagatcgaaattgatagctgcttcctgatattatgtatctctgtagatacacaaactactatatttaaaagttcttatggaaacaaaagacaattttttgcccaaaatgaggaaaataaaatattttattagacacatgtgatcatctcttcatgcatgcatttgaatttaaaacataattaggtgcctaattatgcatatgaatttggctaatatacttgaatatgtaagtaattatggttagttaaaaaggagcccatttccttttcttactaatcaaattaccattcaaaacatgaatttcaattcacttataaaatttctggcagaagtagctttatgtctcattaagatttaacactgatgatgttgaaaaattcaaattttaaaatatattaaaagccagagtcatggagtagaaaaaattacaaggaacccctgttgtaagattgaagagtaaagacatctttgcctctttgtgtccccaaaatcatccaaatatcaagaagattgagaaacagaaatgaaattttcatgtttaagaactctgaaaagctgtaactataaaccacatcacaggactgggcatctaacagaaaacttcctgagaagattctgcctactgtggatctcagacagcaccagcaggatagcaatattctaagataatgcatgttcctggaggaacacaactgtctcatatttgagtcagggaatcagagcaaaggctgattgtggaggcttactaggaccccatgcagcatcacaaggcagaggccctcatttctgcaggcagttggtggggagatagagtggaggggacacaactgctttgttgctgctgatctgggttggctagaatgggataaaagctaaacggaaggttttcatggccactataacttaggatgacctggggaaccttttaaaacaaacagattctcaagcaccatcctcatgtcaattaaatcacaaccttagagtgatccaaggaatagatggcttttaaaggctctccaggtgattaatttatgtgcatgcagagttatgaaccattggactaaagaaatacacacatagacacacacacacctgtattataaaaaccttcaattccatcttgatccattttggttgttgtaacaaaatgtcacagaatcagtgtctcataaccaagagaaatttatttctcacagttctagagtctgggaagtccaagagcaagacagcagcagatttcatgcctggtgaaggctcacttcctggttcatagaatggcaccttttcactgtgtcctcacgtagtagaagcagtaagggttctctcttgaacctccttcataaggttactaataccattaatgtgggctccaccttcatgatctaatcacacccaaaggtcctaccttctggtagcatcacccccatgggggttagaaattcaacaaatgaatttgggggaaacatcagcattctgtccattgcactatcctagaacattaccctgttcctccccaaagtgaaacagtgacagacatctggtctaacaaaaatttatttcaaagatgagtcataattgaaagaatgaggaccacatctataaaaatgtactttgaggtacttttaaaactgaaaaaaaatcatgatatataactcataaaagcttagtaggaaacagagcactgaattacagattcgaaggatgcagcttttctcagtaagtgaacatagaacagtcaacactgagacacatcttagtaaaattgttggaaatcaaagctaaagaaaacattcattaggcatccagaaaaaataaggtcaagttagctataaggaggtacatcagagaggcctgaaacttcatgtcaatatggaactctagaatgtaatggcacaatgcttacaaattctttagggaaagaatgggtcaagctctttctgaatatttttgaagaggcaggaacatagagactatattttgcatgaaatcttcnttgaaaacattcagttaatcaagaatttcttatcatgagctagtctctagtatctatttgttttaagaacaaagctaatataactgtaggatttacaaaatacaatgtaaatattataaattatgaatatctagaagagataagatgaaaaccgggggagatagtgtgattatgttgatattcttaacattcacattttagcagttaaaaaagtaacatttatacaatatatgaaatgtataatagagatatgtgcatacttgtcatgagatgagaatgcatgataaactcagnaaattactttctttagaaataactcaaaaatccaaataatattatctatgaccacgtcccaaaaaatcatttcaatatgagtaataatattggaatcaattctttctaaatcttgataatagttctcaaaagaaaagtaacaaacagtatattttttaacctctgaatagagttatgctatttgctgtagcatgtttaaaattattaggggcacatgactcctttgtggtaaagtttgcttgaagcacagttgttcaaaagaatggccctggggtttagatcatgactccactttatattagtgtgacagccttaggaaagtaattaactctcttgcctcaatttccccatctaggaaatggggtaagtgatagacctaccttatacagttaatgtaaagagcaactgagaaactacacttagagtacgtgtagcgtagaacaaacgcagaatgaactagaaattgggatgcacgggcaaaatatgaatgtatctctgtgttatttgagggaaaatggtgtaataataagggagggcaggacatagatattttcacactcacacactgcatctgatatgattccctttgtttacagacattaactatgcaggttttccagtatcttattcaagggagaggcattggggcaggggcacggtgttttttgtttgtttgtttgtttgtttgagatggagtctcggtctgtcgcccaagctggagtgcagtggcacaattttggctcactgcaaactttgcctcctgggttcaagcagttctcctgcctctgcctcccagttagctgggactacaggcgcccatcaccatgcctggctaatttttttgtatttttagtagagacggagtttcaccgtgttagccaggagggtctcgatctcctgacctcgtgatccacccgcctcggcctccaaaagtgctgggattacaggcgtgagccactgcacccgccggggcacggtgttcttaaccttgcatcttgcctatgttaactcatatctcccatttcattttattccaatcaatgactagcattctagaaggcaagtttttggtgatgtttggtctttattcatcaattattcccctcccctctcacaaatgtcctgacttctgtgcttgtaccaatctctctagaatctgatcaacatttagcccgtgtcattgaattttctagtttgatgaagcatcatgcatatgtttaattttatttatttatccatatacattcgaatctgtgacccctggattggagcctctccctttcagcgcctttcaancaggtgctccttccacagagcctaagtaaagcaccagctggggcattgagcagaaggcacagccatgctgtgatgtgcaggggctccagcactaagtgatttattttccgnacctcctctaattccctgagccacgtgacaactgagatactagttatttctgtctctttcttccatttatgcacctttccatctttatttctggatagctttcctgtggctccttcctaaggacccccaactgactcatccctgctaagacattagcccgtgcactcaagatatcacacactccccatgttgcaagcttcctgagtagtagacataatctaaaggacatcttgcaaaatcgcaaaatttatttagcagacattgagattccgtgattgcagtatgggtggcaaggtgggtaggaatcatacagtggtaaggtttggggattgacaacatcctaaaagccacactgtcgaagtgacacaaaatcacagcaacttaccctatttagaaaatgtgtcttcccttttctgaatacttaccccactttcagttcctacttccgctgnttcccacttgctcataaacatatgagacataaaagctgagacataaaagaggcgacactgtgattttctgtaataacactggaaatgaaattacagaagtttagagctggaacagataatgcagtcatacattttacccacacaggtggggatctgagagtcagagtgagtgggattagaattacagagaactccgggcagaggcaggctgaactcacttctccagatttgcagatgtccaccatacttgctgtttccaccaaaccgtcttacaactcaaaatagtgtttaagaagaaaattcaaaaacaatttgcaaagcttagttttattcctaattttctagtggaatgttagcattatttccctttagtgaaatgctgatggggaagaccgttatccaaacagaggtgatgtgccagttagaaagaaagcaacacaaaatataccatatatgcgaaataaatcacagcaaaaaaacaccatcgaactggtgattccttgtcatgaccttttaaaagaaagtaaaacatttggtacaagagatgagaacaaaactattcaggtggttatttgaacccttcattcattgtacagtcaggtaaaaaataaaaaatgttcagtttggactaaacaacttaatgaatgtcattggatgataaaactcatcctgaaaagtcacttaggaagtaaatcgctatcatgcattttttattagtctttagaatcatcagaataaatgacaaaagcccattttcaaaaaaatccagaataaatgaaattcaactgaaacagcttatagcagaggaagcccacaagcaagataaactatgaatcaagacattctgaaaataactaatagaaaagacatacaacatatgcccttaacactacacgagctgtatatrgtggatcactttgcctgttctatatattcaaaagtcgaaggtaaagccaggaaataataaacactgtattcttatggtgactaattgtccatcagacagtcgttacttttgtaattactgtcattacttaggacaactgttggntatcatttatcgattgcatattatgtgcctggcaccagtgaaaaggttttgcatccattgtgtttttaatcctcaaaatcagcccatggggtatcattatccctcagctgatgagtggggagaatgagataagcaagaatgaactctcccagagcctcttaccagccgcatttggaagccatgtcttgctgccccgtgtgctgaggctgaagcctgtgctcttgatctacaaactactacgttaccagagtagccagccatgagtaaccttcattcctgtggcttgtcaggacacctggtccattataaagtctcattatgatgatggaagaaacacatcttccaaaagagtgacctgccatagtggcatatcctgcaacatggtgacctccctcttgtcctgaagtctttagacaagtcttagaaaaaagaataaatgtgggctgggcgtggtggctcaagcctgtaatccaagcactttgggaggctgaggcaggcagatcacctgaggtcaggaattcaagaccagcctgggcaacatagtgaaatcctgtctctacagaaatacaaaaattagctgggcatgatggcgggtgccagtaatcccagctactcgggaggctgaggcaggagaatcgcttgaatctgggaggcagaggttacagtaagccgagattgagccactgaactccagcctgggcaacagagcaagactctgtctcaaaaaataaataaataaataaataaataaataaataaataaataaataataaagagtaaatgtgaaggaatgtgacgtccactgcaaactgatggccccttaaggggttggtctctggctttaagtctagtcccttgtattctgtattctgtgagacagagagttggtgcataatatatgttagttgaatgaaagaatataaatattaaacaatgtacctgggaggcaatgcagttttataagctactgagctgtaaacatctgggcaaggattagagataatgccagaaaaaattagtaaatactacttttgatgtaaaattaggtggtagacacatcatggctagagatgtgggagagaacttagcaagcaaaagaaaatatgctatttggtatattttagtaagttttgtgtgtatttgaactaaattcaaaatgccgtgataaggcatgtgtttgctatatgtaatgtatagctacatgcattaatttctgttgtgtatttgaatttactttctttgagatatgaatatttaaatttgctttctaaattatcacaaattaaccaacaaatttatacaaggtgattttggaaatccacattaattaatttctttattagaatgtgacataaacagtatttaccttattagtttntctttacgaaaaaattataaangcttttctatatatatcctccttaaaatattagataagaaaactacaagtcgtcagctccacaggagttaacatgtggattttggatgctaagaacaaatttaagatgatgacttgtaatcttcatgaattgcttttggcaacaataaattaattgggcctttcaagaaaatgcagaaaactaaggtaaagggtgattgagtcttccagatttcctatattcccaactcacttctgaagaacgtaattatgagtattgaaatttgggactggtattttttacaagaagaaatatactgtagctgttatttacttttgtggtatttttcttctttgaatttgggtagtattgactataagctatggactcagctccctgtgtgggtcataaatcagttttattaaatattgatttagaaatatcagttattattttcntcactggtggagttctgtgcttcggactacccctgtatcttgctgaaataggattgacaat

SEQ ID NO: 2 - DNA, 237961 bp, Homo sapiens
  exon 43726..43868: exon 8
gatgttaaataaggcgataaaatatgaggaagaaaaatacttttattcaaattataatgggatgtaaattgaattttgttgatgctgctgctaaaaattgattcatcagagttaaatgatattaaagcatatttcatgctaccatatgtgatatcagatgagaactttacaaataaatagaaaacagaaagttatatatctacaaccatgatttattacagaacgtatatttttactgcagtcataaaagaaaaagcccagtaaagactaatttgtaaaatatgtaattgagatttataatgctttcaaaaaaaaaaaagcaccaaggggtttttctgatgcatttatcttgatgtttactgttgccataggaattacttattgatttttatgaaaacacatatggaagagagaactaaggcaatagaattcaactaaaagggcttcatctatgataattctgtttgctctgataaaaattcctttatcatggaacaaggaaaacctgacatatttttgttgagtcgaggacaagatcaagaaacatcctatgaggaatatttgtgaaaactcttagccaaatgcaaggagttttatccttgtatcttgaatcataagtgcccctaggttacatgaaatgtgtgcttgaggatttttaaagggactgtctttaggtttctcaggttctcaaatagggccacagacttacttaaactgaaattaggtgtccagatattcttcagaatgttaggaagaaaggagtttaaaggcagaaattgcaggcatgactttttaaaaataaggttgtccattgtctgctgaaagatgtttaggagttcttgttgaatggatgagaaaagatagtgactagaggagtttttcatcagacagtgaaaggaaattttaaaagtaaattttggagagcataaagtgatgatttatttcatgttatatgtagaagacagtgttcattggaaatatctagtggattccaagaaattagcatttttatggttgtattttaaacatttataagttaaaaaagtaatacttcaaataatgtaagagatttaatgatttaagaaagtaataaattctgccatatataattccctccctccttccctctcttcctttcttttctcatttcttctcccctctctgtaagtttcatggtatgttataacatgtttttcattcttcccttactgatagacatttaagtggttttcagtctttccctgcaataagtaatgctgtgttgaatacaggcatatatacattctgtgctcatgtgtgattatgctttagaacagattcctagaaacaatttctgggtgaaatgatatgcacatttttaatatgagatagctggcggtttacttttttaaaagataaggattcatagtactgcctttattgcgtgagtgaatttttataaatacaaggtattctttttcaatatgcataaatattgaagaaaaaaaataatgtaggcatttcgtattccgtactctgtactcttctgaacactggcagtccattctcttcttgaatgtatgtattcatcccattcttcctggaatgcctctctccattctcttcttgaatgtatgtgttcatcccattcttcctggattgcctctctcctaccgctccccctacccccatctcctgtgactcacctgtcaggacctactcaggtaagtttcccctaatctgccagcagcactcatctcccttctgtctgaactgtgatagcaaggtttgtgcatcaaagtggacacttcgttacttcgttctacatgtgtcccaccgtctgggagtgtgtgtgtgtgtgtgcacacgtgcgtgcctgtgtgtgttttacccttatgttctggattcgttcattagacccagttgtctcaggtgggagcagagcctcaggcacttcttttgatgtcttttctcgctctcattgaactcggtatatacttgaggactggctgatcacatttatgagaattttatatttgatagattgccttttgaaaatcatcattctgcagaaatttagtaattgaaaatttaaatcaaattcaaatacaactctaggctctaatttcactgttcgtttgaatgtatgacttctctgttaagccagatactcacgtgaaacaaatgtgaatagaatagcatggaaatcgtgtatggacagtcaaggaaactttctctgctggcgacagcagcctgcagaacataattccctggacactctatgaaatcaaatccgttttatcaactaatcagttggagcaaaaaataataatagtgttatttaggaaagccgtaacacagctcttacctcaaggctaatgaagacctgacccttgaactctaggggcttccatgttccagcttgtgagccctgctttgccaaaaccctgctgaagaggtaagagcctccttctctggagagaaagcccccagcctttgacaaagtgcttctagcctgagccatgtgggctatgaaaaagaagttggtcatcccgaggagtcactggacagtggagagagcccatccatttttctcctttattcttccctacccctcagctctcactcccttaggacttgggattctggaatcataaaagcaaaatattctaaatatactaaaacatgctaaataacttaaatatgctaaataaaattaaaactaaaataaacgtattagaataaatcatgaatggctaataaatataagctgccaccattaaaacaaaaataacaaccactaccacaattgctactataaagattattattgatcagctttatttttaggtaagtagaaaagtgagatgtatttctatttttctgagaatgtaaccattctgaaagaataggagatactttctcacagtaagcagaagaatgagatgtactcataaataatatattcaacaaacagttattttgcacttactctatataggtactgaaaatagaatgatgttaataaatatgaacagccctcaaacagagatattaaaataaaacaagaaatgaaggtgaaataaatttagaagaaacgatggatcattaacttgtagtagttcttacagccatgtgctggatgcaggcatccctgtctgagtgtgtgcgtgtgtacagacattgtttccaaacttttttactataactttgttaaagtgaagcgtgagcactccacatttgctgcaagtctcacttcccctatttcctttatttctcacatttacgtaacttgtctaacctctgaaacaaatgtttttattagttctggtaatgcaaaataatctttagagaattacgtttttatttttgcttttgttaaataagatctacagtagcataagttaagtgtgccaccttcattttgtagctggctaaatatttctgcttcctgtgtacttttctgaaaatggctttaatttgtcttatttttgataattagcttcaattagtgcagaattctcctttggtattgattggacataaaacctgagatatgttcgttgtattctggaatctattactgccaataacaagcctaccgtgagtcgaattttaaggctttcctttttttttttttttttttttttttggctttgtgaatttgggttttttttaacatcgtttttctctggtagtttttaagatttaccctttgtctttgctgtcttatggtcttagtatcctgtgcctaggcatggatttttattttttatctactgtgaactcattctctgcatatgagactcatgcctttcttcaattctggaaggtttttaggtattatcttctcaaaacagctctcttctccattctctctattctttgttttctggaatactgattaatatttgtttttatcatattttttattctatcctccatgtctttaaatcgtgctttctaacttccctctattttcttgacttctcttgttaccacacaatgggcgggttcagttgcttggcaggtgggagtccaatgaccacaaccaaggaggatttaaccaagggatttcattactggcaacaagtaagtagaacactggggatagttcccaaagcagtgcctccccagacacaggtggaaacagggctttgtgggggctgcttagctgagtcactgtgtgcagaggtggagtgcaggcaggcacagctgttgttcatgcttcttcatatgttgcccatacagaaaatggcacataagctcttccctgggcagggcatttagtatggtaaggtgaagaggtctccaaagttcatctccagctcaggcatccccggatgcctgggaccagttttttgtgtttgttctttgttttgcaggggctcggcttcttcctagaacacttttaaacaacggaaactcaaggtgcaacagttaaaagtggatagttcttcacagtgtgtacccaaaaacccagggaccctgggttacactcttgatccttgattagttgcacaaggaatcttctatcattctccgtccctccaagagttgtttacgtattttcaagctcaaattctttgctgaactcttaattttctctcagtgtgtctagactgctatttatttatcgaattttcaattccaacatctataatttttatacctaatatttctatctaattctgtttcaaacctgcctgttcttttatcttagattcctgttcttgctgttttctttctaatcttttcttcatctcaccaaacattttaaacatgcttattttagaatttgtttctaatgcttccatattttctaactctcctttacaatagttcattttcttgtgtggtttataatattggctatgagctcatttccactggctatgagctcatttttcaggggtactctggcctgaactgtgactggggttgtgtttgctgcagtttcatggatatcaaccgttcttactgatactcgtgttacttttgtggcttaaagaatatattaatgtgttctcatgctgctaataaagacatacttgagactgggtaatttataaagaaaaagatttaatagattcacagttccacatggctggggaggcctcacaatcatggcacaaggtgaagaagtaaaggtatgtcttacttggtggcaggcacatcttacatgggggcaggcaagagagcctgtgcagggaaactgccctttataaaaccgtcagatctcgtgaggcttattcaccatcatgagaacagcacaggaaagactcgcccccatgattcaattacctcccactgaatccctcccaggacacatggggattattatgggagctacaattcaagatgacatttgggtggaaacacagctaaaccatatcagggaaggagaggtccttgaactatttgcattatgggggcactgtaaaattagactacacacttcagttggcccaggtctggattctgatttctctgaggagactcttttgccccgcatggatctggacagaagtcaagcaacattctgcttttttctgagctggaggccagggttcttccagtccagataaaatgcagaacaatcctttgtaacacccagctttgtagagacacctcagctccaacttactgtcttatattaggtgcctcccaactcctctctctattgggacccagaagccctgaagatttatattgactccaagtacatctgtgaaccatttggcttcagctgctgctcacagctctgattttatattctcccaaactctctgaccgctggatagtttgctcatgtagctttgagataatttatacattcaaaacttactctcttttatctatttccttgtgttgatttggcatagggtgaggagggtaagggaaaaaggtgtctaaataagcatagtccaacacattgacctaaagcccatatatatatatgctgctaataacattatatatatgtatatatatattatatataattttttttgaaacagtctctctctgtcacccaggctggagtgcagtggcatgatctcagctcactgcagcctcctcctcccgggttcaagcaattctcatgcctcagcctcccaagtagctgggactacaggcgtgagccaccacgcccggctaatttttatatttttagtagagacagggtttcaccatttcggccaggctggtttagaactcctgacctcaggtgatctgcccacctcagcctcccaaagtgcagagattacaggtgtgagccactgtgtccagcctatgtttttatatttttaggattcccatgtaatgatgcaattttcctttctttgatatgatgcatctatgattttatattctaaatgtatttttatacttatatataaaatgcattttattaattaagcattagttcttaaaagttggtatttccttgaacttttaattttacaaatattccatttcggtgttattatttttagtattttttngctgctgaatagttatgccaacctctgtgcagcctgggaagacaacacatcagcagtcaatttgtgtaaagacaaaataatattagagagcaagagaatgagacaagactgcaaatcagcaatgataattattgagtactgagaacaattccagtggattaagatttgggatgcttaagcttcttaatattaattgcagcaaatattcaaatgctttcactttccatataagtacttcagcaaataagcaaaatatgaaataaaaaccctaattcagaattacctaaatataaaacaagtaaattgtcctaggcaattcttaccatcttatgtccagtaaaacaataacctatatttgtgcatacagatatattatctcatttaaaaactagcggtgattgtaaatcttgacaccaaattgaagttgaactgaaagaggctttcctgtaaagatacttaggatccttacattgtatgtaaggaaagtcacctagtcctcacatcaaccccaggtgtgacatgttattaacttattctaatggtacagatgttgagatgcagagaacagaagcagctcttaggggacagagacagggctcagtgatgattatattcacctgtcatggggtagagctacaatagtgagtattctgtggatggtcctatccagataaaagaaggaattggtttcaggacatagtctgcatcttactaggtggggaatgttggacaagttacttaacctcttctgccctgtaaaatagggataaaaatggtacatatcacagagtgtcataggaagactaaagtagactattctcatcctcacacctaatgagttctcaatatttaactgattataattatctttactagatacataattaattaaataaattaaggaattatagagcaaatagtcatagggtcacacagatagaaagtgggaaaaataggagacaaacctatgtctatattcattccatggcattagatttagattacctccttttagtctggcaaaacactcatgattatacaatgtaatttaaaaatatacagtactttgctgggtgtggtggtgcatgcctgtggtcccagctacttgggaggctgaggtgggaggatggcttaagcctgggaagcagaggttgcaatgagccgagatcacgccaccgcactctagcctgggctgtagaacgagatactgtctcaaaaaagaaaaaaaaatacagtactcacattttactttgctagtatatgctgtattcactggtgtatgctctcttgcatggagactttcattgggaataaaatgtttgaaactattttactatttcttgcactgctttgagatactaccagcaaagctatttctaagctaagacgctatgtcctggcaggacttctaaagacttaaagcaatgctgtagctgtgtatttacaactgactagagcagggatcagcaaaccatggccagtgggtcaaatcctgcttactgccagtttttgtaaacaaagttttatcgcaactccctgcccatttgtttagacatggtctgtggctgcttttctccaacagcagaaaagttcagtagtttcaacagaaaccctgaggcccataaagcctaaaatgtttaccatctggccatttacggaaagttagccaacccccagactagaataaaggtaggaaaaaccactcagcccaagcttcttttttgattctatcttcccacagctgcctagaatcctttgcagtctttatcctaggaggctcagaggctataacacatctctaattttgtctagatttgtgagaatccatttctcacaaatccagttctggccctctttccctgactcgatgttccttgaactctaaacatgaatttttaaaagaaggattacctgggatgggtatatccctgaaagtcacctcctttctgctgcctctcacaaatacacaggctatgtgccttaaacctgtcattcactttaaggaatttttgaaaagtggataataggttagacataaaattcactgaatctgggttaaatttaatcagtgagtattctttagaatggaagcatgagcctgaatgattaaagaaattgcttaaaaaattagaatcaaaatactaaatatcatcccccccccaatgttttagctagaaaaattacttgaaaatgagccagagaaggagaaaaataattatcttacagaaaactaataaaaatacaataaactatcatttatttaaatacaataaactatcatttgttaaaatacaataaacatgcaaaagcgctatcattgatagcacttttaatgtttaaataacccactgattactttctctagtggattatttgcaagggtagagtaagaatcgttgcttctctggacacataacccatttgccatgtgacattgcctcccttccaccaagaagtggaatgtatttttcctccccttaaatctgatgcctataactttctttccccttgagataatggcagagtggcattgtgcaagttttggaactaaggtcccacgaggccctgcggctctctctcttgtttatctgagtactgccctaagatgaccatggagagaagcccagtctagcctagaaaaggagaagaggccacatggagaactgaggtcagccagccccagctgcttgacttctgaacaaagccttctccgactatttggccctgttagacccagctgagatcagcagaagaatcacccatctgaccctagcccaaattgccttctgcagaaccacgggccaataaatagcagatgctttaacccaccatattttgcagtgatttgttatatggcaacatatcattgaaaaaccctcattttaccaaagaaggggggtcatgacattaagcaactacctaaacccacaatgtaaatggcaagtcagggatttagatgcaaatcattccaactaaaaatgtatactctaaatcaatgccctgtactatactctgtattttccaaaaactaattagattgtgtcatcagcatttctgaagacagaggtagtcgtctgggaagtagagttttctcaaccagtattttgcacttgttgggatttttgagatcaggaatgttttggaaaacctgagatgtgcagtcacatctctgagcagtgttttaagtttattcattcactcattcattcattcagcaaatatttattgagatctactatatgttaggcactatgtttaccactggaaggcaggcaccaggtaagtacaaacacagaaactgagatggatggcattgcattttcatttgattacaaatttattcttgcttataagttgattacctaccttattaagagctgatttgtttcaagttttagaactccaattgtgtccttaaatttttgtctagcttaaaacatagtgctagatttcaagaagtaaattgtcaaacaggtttgctgtttagtttttgtaaaaaaggaaacttgtaggcagaaacaaaaaccaaaataaataaaatctaaattatggtaataactaaagatagttgaggcattaacagagtttgactgaactctcaagtctcaagcaaatttctaaactaaactgttttcttctgttaatcagtttaaaagggaataccttgtgctaatttttcttggtgttggtaacattaataaagctctgtgaaatgaggccagaaggaacagaaattgagtcaaaataatgataacacctcttacatattcttaccatcttgtctcctagccacacacaccatgtttaaacctaaaaggagagacacctggaggggttaattagcttgcagctgtctgtgactagtctgcagaggataaaacggatatcagccgtaagaaagcagctagcactagcctggatgatgaacaaagaaaagaagcagctgctgaggaaaagcacattttgctgacctttgaggagggcttaagaggccctttgccccctgaaaatgggacctgggaagaaagtcttgatggcagacagtacaaaatcaagggacagcagctggctgttttgtgtaaggggcaaaattcaagaggatttgtgaatcattgtgatttacagtgaccattaactattgtaataaggagagaaaaatcaacatccattcagtgcccacattgcaaattgccaagcactggaggggtactttcacctaggccaacaaacttaataagcagagaaacccttcagtgttttggctcctacagctcaagtgtactaggttggcaagtggaagttcctttggtcatctctgtttatacacttgtttgagatatttcataaagtcagcccatttgttactttatctgtagtatgtgctgtttcatgtggaagatgcttgccttttgatagtattccaaactgaccaaccctgtacggatttaagccactgagaagctatgaacagacaggtgaagttttggtattgccaggaaccagcttgtccagaagtttaatcctttaaggaaaaggatagcaagcccaatgcaacagcccttcatgctaaaaactctcaataaattaagtattgatgggacgtatctcaaaataataagagctatttgtgacaaaaccacagccaatatcatactgaatgggcaaaaactggaagcattcgctttgaaaactggcacaagacagggatggcctctctcaccactcctattcaacatagtgttggaagttctggccagggcagtcaggcaggagaaagaaataaagggtattcagttaggaagagaggaagtcaaattgtccctgtttgcagatgacatgattgtatatctagaaaaccccatcgtctcagcccaaaatctccttaagctgataggcaacttcagcaaagtctcaggatacaaaatcaatgtgcaaaaatcacaagcattcctatacaccaataacagacagagagccaaatcatgagtgaactcccattcacaattgcttcaaagagagtaaaatacctaggaatctaacttacaagggatgtgaaggagctcttcaaggagaactacaaaccactgctagacaattgttaaaaactaaagttgttggggaacaaattgtacttaaaatatatttattgtgtaatctgataaaatattttaaattctggtgttacaaagatttagtgtttcttttgttctttttacaaaacagaaacccactaaaatgttaactgattgacattatattttttgctttcgtcagtgttaaatacttgttgcacagtagtatgagttgtttctatcatttactcttctacaagccagttgagacgtgttacgaggactcaaaatattctaattggcggtgaccaaatgtcatacaattatgaagtatacaagccattctaaccttcacagtaaatttttttaccctctggttgcatacttaaatagaattatactaataatgcatattttctgagtgttaatgtcatgtgaaattgctactaagttaaacgttgcttcccatggttttctgggtgatgggtgtttattctgcttattcagtgcctctctacatagaggaacctatctcagtaactcctatactactctacaacaaaatggcagaagaaaagttacccatttgaaaaaataccttattttccaaacagaagtcttcagacaggcagtgcacaatatttgccttccagaaaaggagattgcataaatgcataacttacatgttagtacatgtgactgtttccctgtcttatcctgaatgcatggaaagtcatgagacttatttttttcttcctactggattccattataaaacctctatttgtataggttataaattctaaccaataattgagcttactatgttgaggctgtaggcagaggataagatttacatgtcagactttatgttctgctctttggattaggctaattttcaaaagttattgactgtaaaataataatcatgcactgaggaaatacaatattaaaataaattttgaagaagatggaattatagcaggggacctgtggaaatagaatctaagaacatattgtacatatgctgaattaatagagttgcattgtgtcgttcacatagcaaattcagtacagattgtttgttaatgtcaacagattatgccgtaaaatttgtaatttgtttggcaaatcttgacaatgctgagacatttattctagttaatacaaatggctaaggtctgtaattatgagttatttaagctaagaaatttggcaagttcattattcatcagtctctacctactattccaaattaattgactgttttatatactgatacaattaatagtaaactttttatattaagtatacatatgtgaaataaaatcatattattattggattataagatgttaataatggtaagtcatgactgttattttgttgaagtgattttttagaatttttaaaggtttattcaacaaaatagagttatcttacttaaaaatggatgcttagggccaggcgcagtggctcacacctgtaatcccagcactttgggaggccaaggcgggcagtatcacctgaggtcacaagtttgagaccagcctggccaacatggtgaaaccctgtctctactaaaaatacaaaattagccgggcatggtggtgggcacctgtaattccagctacttgggaggctgaggtaggagaattgtttgaacccgggaggcggaggttgcagtgagccgagatcatgccactaaatttcaacctgggcgacagagtgagactctgtctcaaaaaaataaaaaataaaaaaacaacgcttagagatttttaaattaaattagtttcaatacttctataatagttaaaacaagtggtttctatttaaatcacaaatgatagaggttagagatgaaactaaacatatgagatcatcaagtccaaccttctgctacgatattagacttcaaatggaaagcacatcaaacgttaaccagatgatttctttgatagtctgtttaaaaatattgaattaataaactggaaacattttaaaaattgctgttcttttggaattaatataaaaacatatactttaaagtaaggaattttatactgattaagttatacactgcttagcctagcatgtgtatgttatacttaaaaatgtgcattgtacattgtcaaataatttgcacacgatcatttacacatctgtattgttcacattgacgtactttgtgtcatatatatttgtgcattatcagttgatttgcacattgcacaccagagtcacacagttatagatctgtgtgaattccctatattccacatgcatggacatgttcatatatacccacacaaatacaccccatgaaatgatatccagttaatgaatacaaaggtttttaaaggagaaattcattagaaaaaaatcctataagcctctagttttcccaatgaattaatctaagaatggatattttattttttccatgtttgacttgtaaagtaaaactttagtaataaagagtgtctgccttacagttcatatatttcttgaatttaaaaaactcaattacataagaaatacattaattcttttctttaaactattaagacagcaaagtcttttttatgattatctacaattccggtcctctctccggtatctccaagataacatgattatgagtttaatgtatgacatttaaaactttgaaactcactttcatatngntntnnncnnnnnnntacatagaatatatggagtgttgtttacatattatttatatgaataaatatacacctactccaggtaatttagtggtgactatcacgtattttatttggtcattctacaattgattgatgccttttgtgtgtgtgtgattactaatactggagtattaatatacttgcatgagtctccttgtgtaccatgggaatagttctgtatttgaaaatagaattactgggtatgcacatttttaatttttaatatatacttccaaattgttcctgcatactcccaccagtactaagaatgtatctgttttcctcttagcatgtcaatatttgatataatgaaacttgtaaattttagccaactaaatgagtgtgatttctattcctttatagtaactgatgaggttataatcaagttaagcatttttttatatgtttattggccactcaggtttttttcttctacgaattgctggttcacatgcttcgcccattttccttctggacttgtgtgtgtgtctttttcttacatatattggtatatttgacttttgtctattatttattttaaggttataggattttagagttaatgaaaggataaaatgctggaggcctagaattcagaatcgtacataggattctaaacagaagccatgttctaaataaggatttggcttgtgggacaaaagaaaaatgtggaatttcacagnnnnnnnnnnnnacatttatgggcaatagagaatcaagaatgactttgtcataacatcggcatcattgaatggtattgtcacctataccatgatcaagaaattaacggtgttgctctgcagactggcaaatctcattgccctttcttctaaccctgggaaagtattattacaatggaaaaagatatgcgacaagatattgtattaatagggtgttagaatcaaataaaaggagggatagatgtagtaagagaagcagattttatagctatccccattccaagaataaaaaacaaaaaaataataggcgagttcattgtgggcatctgtcaggaacaaggaaggatagagctctgaaagctgactgcagtcactaacaaaaacccttttgagacatattccaagaacctgaaaaactggtaatggacccttccttatactcggtgtcttgaaaggcagtaaactgtctaatgggatataaaaatagagtctttttttcctcccccactctaatattctcctattttaaaaaccttctttatatggcactgcaaataatagcacacagcaagaactgtactttgcagtattattgagcatctcaagtgtctgaaaggtacaaagctaacaaccaaatgacttcagaatggaaaatgtcccagaatttttaattttccttctaaaacagaaatataggggaagcaaaaacaaccttaatctaaaatccagcttaaaatgacaaccagaggttttctagcttatattttatttcagaatgcttagcaaatttcttgactttgtattaattttagtgagttgtaatttgaggaaactcatggttttctaagatcctttatacctgaaaagttgtcgcatacgtgtaagacttgctttgttattgttgttgtaaattgaatacctgaaggtagattttagtacactaaataatcatgatgttcattctgtgctaataggcaggaaagatggacaatagaacttactgggatgccaggaagtgctctgatcacccaggagtgagacatgagatgggcagaaagaacaagattagttaactatcatgggaaacataagaagaatttggctttagggattgtgtgtttgagattgatttttaaaagaaaaagacctatttaaaaatccacagatgcttagggaatagaatagctatttttttaattcataaattttatattggttagttttcacagcagtgaattatcaactttatataaaagtaaaatgataatttgatatatttcctgatatgagtccacattttcagagccatctaaattcagccttgtttgttcattatacatatataagaaaatccttcttctatgcaacaaatggctgtatataaaatatacatgacttgttgatttaaaatcttttacctttttttctcatcataaaggtaaaacacatgcatttcataaatgtttgaaaatgggaacgtatgtaccaaaaaaatcacttcaaaatcatggtttacgtatcttctaaaaacatgtaatcaaatataacaaagtatatttaggccaatattttgactatgataaaatcgctttgattcagaaggtattgtgttttctgagtatttcattatttaaagcagaaggtttcatctttcagaagtgtcactccagcctgggtgtcctgtgtggttgagtcaaatcagtgcttccactatcagtaggtttggcacaggatgaagcaagaacatcaaactaaaggggccaagaacaataggtgggagaggctgcaggaggagcagagaggtcctgataatgaggttccctgactatagcaggttaggactagacaagccctgtgaccaacagaggaaagagtttctgagcactttctatatctggctgtaccttgggggacttcctccattttgtctgggctcatccacagtcctgctaagagattctgcaaaatatactgcagttctcattcatgtgccccttgagatgcatttgagctgcaggtattttccctccagtggtacccagtttattatttctagtccattcaactcataacaacatccagcagcactttacccacaggtgggtgtttattaggtttttatttcttttgtccaggtttattgagtttgaagagtaggcagggatttctgtttttatgagtcacttgtcagctgtaatgaagtcttcttcactaaaaaaaagatgtgaggacaatcttgcgcagtgtcataattattctaaaaatacatcatgcttaattgtatccacaaaggttttaaaaatttaaacatcagttttgagttttagggctttcttattggtgaattatcaataattgtagtgctttcttgaaaagttccctggggtggatttccttagttgcatataattctagcctcttcagcctactaaggataaaagggacatgaagtacacatatctgggaattaattacatttttctgctcaagagccaagtcattttcagaaattgatgtcaaataggtcttccatagctgaaagtgctaggtgtctcttcacatagttaagatttccccctattcagaaatattagtttctgatgacaatggaaaattccaaatgcagcagcttcactgtgttctaataatgttcatgcttagggatcctgaaaccacttgcagtatgcctggtctagccacagcagggagccggcgcccatgccagttcccagagctgaccaccccnaccgcagccagcgtgtctggctgtgcaccatggccagactccatgctcactcactcatgcaccccgcactgctccccacctggctcaaccttggtgggcatgggatccaggctggtagtgaaagtcaaatgctgcctgccaggccgggtgggcagaacgagcccagcgggtgagaaaaacttgggcaaacgtgccactggctacagaggtttccggctggtgaagtgacaccctaaggattctgtgacaatgggaaaaggggtaatgaagggatagagcagattaaggaatttaaaacccagttgagcaagtatatctgtaaataatgtacttttgtatactacaaaaccacaaagaaaggatctccctcctctattatcacagggtcatcccggaagaactgacatttcaatacgttaaaaaataagtagataatgtttttagctttaaaaaaaagtggaataatctatgcatatataaagaataaatgcacataacatttatgtgggttatgtaagcagaggtaagcattataataaagcaaacactgatgagccactggtgaggaaacataaccttcctaaatctctgaggctttcagagtggctcttggcccatgtagaggtaagggtgtgaaagaacaggaagagagggcgtccatgtttgagaaaccacaaaatcacaggagtcgctaaaggagtagtttaattggttatatggaggaaatctggagtgatgttagaagatgcaattgaccagatcaggtggatcacagaggccatcctgagcagtacaataggaaattaatcagggatcccctatggttaccataccttatgggtttaaaccaatgatgcttacgagggagaagttagcttttctggacaatgtccttggtgtgcacttttcagatggagattggcaagccaatggctaaggagggtgaatcagatcaaaattcttccttgactttcatagcatttccagaggatgtctagtttatgagtaaaatgtcttgccttcatctcataaaattatctctatgtcctcacctgtaaagatgttctatatcanaacattattatttatacaaataattatattttcttaaaaagacttttttgtacagtaaaataagtggaagcaaacttaaatgattcagaacaactttctaaatatttgactaaacaatttcaggaaatagccatttaatccctcgaaaacaaaatttggcttcggttaatttttagaaaatcctaagatgaggagatgaagctaaaaaaaatatattgaacactgtgcatttcaataaagaatttacagaatagctggaaaattaaggttaagtatacaaggtaattatatatttatttttaaaatgaaaaggcatttaacaatctaaaaactacatcaaatacattatatttagcatcatgaaactacttaattatctgcacctatcagtgaagcattttttttctcataaatctgttctatgataatctacaggaagacataattttaaaatttgtaaggattagagtggatggtcacctggtcccaacctttggaacatttgaaaagcaaggaacatataaactgaacattaattttctccttgcaatgtatacaagaaatttaaaaaagaattttccagagtacaggtataaaagggtatattcttgacagggaatcagagtgctgacagatggctaagatttccatccaagaaaaatttgatgtttgaacttaagaaaatggaaantttctttgctggacctcanataatattttcagtaatgacagaaagtcaggaaagatgaatgtggaaacaaaatgttcttgaacacatgaaataaataggtaatttagatacagtgggtggtgccagtatgctctgtaggtgaagaataaatctggaatcctaggaggtcgccctaaccagtgaggtctctctcacttatgaacataaaatgtctgggtacttaggactcctctaggcaataaatgaccttcagttagattgagggctgatcgaactgagagggattagtacataatttaatctatttgctaattagcatgatgaattagaccattgtcatctgttgctgtgtgggagaggaagaggacaaggcatttaagttttgatgagaaggacatttattgaatagcattgtacactttaagagtggattctcatatatgatctaagtatcttagtctatagactaagtttgattctttcctttcattagtgacagagctacaactgtcacttgagtattgggtgacttgacttctcttccaagtcgaacacactgcataacatggaaagaaggcaaggaagaggagaatatatcttgtcccatcactggtcatttcaggggagattttggcactgaagacagatcaggagcatatcagataggaacaaaaaaaacaggcttggtagcttaaaaggaggagtcaagtcttaccaactctgaacactcacctcagtagtgatgtttcatttttaccctcttagggtaaaagagtctggtatttctaagtatatgctttctagtgtttgtgtttactatatttaagaaacaccattagatcaattcttattaatgattacttgatattaatcattatgattagacactagtttaaaagcatagattagtttcattaaagacttttcttgagagattatatattaaatgggtggtgtacacattcttactaagaaagttttctgctgtttatatcttaaattaatgtagaatttctcccttaatttctacatagaaacttagtatagataagcttgtgcagtcagctgtgaattcatctaggcctactaccatttcttcatctcctctaaagaataaaatatgaactatcttgttgaaattaagtgctactagatctatactgctagtgatcctgtaatatcaatacatgcatgcatacatacataaatattttaagaaggaaaagaaatctggttggaactgatgccttcccagtgagcctttgctggctcttgacagttttcgctctcttctttaagggtgttcagattatctagactttctagaattaatgattattttatcgttttgacaatttttctttctaagttaaaaccacattggcattttgtgaatatgatcatatttctcattttgaccaatttttccctagcattacagttcgttaatttactggtaattaaataatgattaaacatattaattaaacaaataatgattatttatttttgtattacgttttattccttaaagcctctctccatcgttaacccttttcctttgtnnnnnnnnnnnnnnnnnnnnnnaagcccttccttagcatctcactggaggtacgttcaggcagcatcactttagtatctcgccagcattgtcctcttcccgccagtgggaaccaactaacaagtacactctcaggtatgaacttaagtacaggcaagtcaacagatttcatatgcatccaaatgacacgttaaatacaaagctttgctcacctgcctttgtacaggagtctgctgctgctgggttttaatgaaacttaaaggctgaatcaaagccttattagtgggaggatctttgacatgaaattctttttgttcagtataatttgcattttgtattcatttgaaaaattgtcctgtgcatttgttcgaatatcgagaatagagaactgagtgattcctgggtttatgtgggtgatattgtggaaataagaggtgctgtggtatgtacagctagtccccaaatatgattgtattttactagagatagagaattcacagtgactngaactattcaggctgtaaaatataggaaccatctgccatagaataagcatattaggaaaatgccacctattcctacattagtctggcccttgctccaaaatttcacatgtactcatatgtttgttttccgtgtctctgattgatgcagctagctaatgcctaatgacagtttttttaatcattccctatatacatttttttattttattattatactttaagttttagggtacatgtgcacaatgtgcaggtttgttacatatgtatacatgtgccatgttagtgtgctgcacccattaactcgtcatttagcattaggcctatctccaaatgctatccctcccccctccccccaccccacaacagtccccagtgtgtgatgttcgccttcctgtgtccatgtgttctcattgttcaattcccacctatgagtgagaacatgcggtgtttgattttttgtccttgcgatagtttgctgagaatgatggtttccagcttcatccatgtccctacaaaggacatgaactcttcattttttatggctgcatagtattccatggtgtatatgtgccacattttcctaatccagtctctcgttgttggacatttgggttggttccaagtctttgctattgtgaatagtgccacaataaacatacatgtgcatgtgtctttatagcagcatgatttataatcctttgggtatatacccagtaatgggatggctgggtcaaatggtatttctagttctagatccctgaggaatcgccacaccaacttccacaagggttgaactagtttatagtcctaccaacagtgtaaaagtgttcctgtttctccacatcctctccagcacctgttctttcctgacattttaatgattggtgtgagatggtatctcattgtggttttgatttgcagttctctgatggccagtgatgatgagcattttttcatgtgttttttggctgcataaatgtcttcttttgagaagtgtctgttcatatccttcacccactttttgatggggttgtttgtttttttcttgtaaatttgtttgagttcattgtagattctggatattagccgtttgtcagatgaaaaattttctcccattttataggttccctattcactctgatgacggtttcttttgctgtgcagaagctctttagtttaattaaatcccatttgtcaattttggcttttgttgccattgcttttggtgttttagacatgaagtctttgcccatgcctatgtcctgaatggtattgcctaggttttcctctagggtttttatggttttaggtctaacacgtaagtctttaatccatcttgaattaatttttgtataaggtgtaaggaagggatccagtttcagctttctacatatggctagccagttttcccagcaccatttattaaatagggaatcctttccccatttcttgtttttgtcaggtttgtcaaagaacagatggttgtagatatgtggcattatttctgagggctctgttctgttccattgatctatatctctgttttggtaccagtaccatgctgttttggttactgtagccttgtagtatagtttgaagtcaggtagcttgatgcctgcagctttgctcttttggcttaggattgacttggtgatgcgggctcttttttggttccatatgaactttaaagtagttttttccagttctgtgaagaaagtcattggtagcttgaccacatagttggaagtaaagcactcctcagcaaatgtaaaagaacagaaattataacaaactgtctctcagaccacagtgcaatcaaactagaactcaggattaagaaactcactcaaaaccgctcaactacatggaaactgaacaacctgctcctgaatgaatactgggtacataatgaaatgaaggcagaaataaagatgttctttgaaaccaacgagaacaaagacacaacataccagaatctctgggacacattcaaagcagtgtgtagagggaaatttatagcactaaatgcccacaagagaaagcaggaaagatccaaaattgacaccctaacatcacaattaaaagaactagaaaagcaagagcaaacacattcaaaagctagcagaaggcaagaaatagctaaaatcagagcagaactgaaggaaatagagacacaaaaaacctttcaaaaaattaatgaatccaggagctggttttttgaaaagatcaacaaaattgatagacctctaacaagactaataaagaagaaaagagagaagaatcaaatagacgcaataaaaaatgataaaggggatatcaccaccgatcccacagaaatgcaaactaccatcagagaatattacaaacacctctacgcaaataaactagaaaatctagaagaaatggataaattcctcgacacgtacaccctcccaagactaaaccaggaagaagttgaatctctgaatagaccaataacaggctctgaaattgtggcaataatcaatagcttaccaaccaaaaaaagtccgggaccagatggattcacagccgaattctaccagaggtacaaggaggaactggtaccattccttctgaaactattccaatcaatagaaaaagagggaatcctccctaactcattttatgaggccagcatcatcctgataccaaagccgggcagagacacaaccaaaaaagagaattttagaccaatatctttgatgaacattgatgcaaaaatcctcaataaaatactggcaaaccgaatccagcagcacatcaaaaagcttatccaccatgatcaggtgggcttcatccctgggatgcaaggctggttcaacatacgcaaatcaataaatgtaatccagcatataaacagaaccaaagacaaaaaccacatgattatctcaatacatgcagaaaaggcctttgacaaaattcaacagcccttcatgctaaaaactctcaataaattaggtattgatgggacgtatctcaaaataataagagctatctatgacaaacccacagccaatatcatactgaatgggcaaaaantggaagcattccctttgaaaactggcacaagacagggatgccctctctcaccactcctattcaacataatgttggaagtcctggccagggcaattaggcaggagaaggaaataaagggtattcaattaggaaaagaggaagtcaaattgtccctgtttgcagatgacatgattatatatctagaaaaccccattgtctcagcccaaaatctccttaagctgataagcaacttcagcaaagtctcaggatacaaaatcaatgtgcaaaaatcacaagcattcttatacaccaataacagacaaacagagagccaaatcatgagtgaactcccattcacanttgcttcaaagagaataaaatacctagnaatccaacttacgagggatgtgaaggacctcttcaaggagaactacaaaccactgctcaatgaaataaaagaggatacaaacaaatggaagaacattncatgctcatgggtaggaagaatcaatatcgtgaaaatggccacactgcccanggtaatttacanattcantgccatccccatcaagagatttttttacacattccatatgtgaatatcctattttctgattcagttctgactttatttccattatatgtgaatgtgtttctttactatttttctatcagtttccttcctggtcaattgactatatgggaaaatattttatccattcaatattcaaaaatagtttattgaggacccactatgtgctaggcatttgatcacaacagtgaaaaacagagaaaaaatccttgctttcatggagtttacaaattctaccaaaggaaacaggaaataatgaaaataagtaagtaaaataaatagcatgctacatagtaataagtgtcaaggagaaaaggaaagcagacaaagagaggggaaggtgtgagcaggaattgcagtttcagataaggtggccatgaaggtgtcccttcaaggaaaatgtagtagttgggaaggggcaagtcacatggctctctggggaagagtgtgcagggcatcgggagcagcccttgcaaaggccttgagaaggaagcatgccccatgctctcctcctcatatgaggaggccaacaggtcttgaatgaagctatggagaaagagaaagggaatacgattggaaaggtaacaaggaatggatcacgtagagccttccagggcaacgtaatgaccttggctattccttttaaatgagatggaaagcccgtggagcattttgagtagaggcgagacaaaactgatcaggacaactctgcttgctgggcagagaatagaatcaagggaagcaatgacagaagagactgtcctaagagtccaggcgagagatggcagtggcttggacaaaggtggtagcagtggaggtcacagaagatgctgaatatagtgttcatgtttactttatgggatttactaagagattggctgtgtgttaaaaggaaaagatcattctaaagcttctggcctcagtgattagatagacttgccattaacttccagaagagcatgtttgaggggaaatatgaggatctcggtttcattacatgttaagttccagtaatgtgattagattgaaaatgctatagctggtagaattttttcactttttatgtcaaaacagaaaacatgattgaataatgaaaactacacatgctcaatttttttaaaattgttttctttgacacagtaaaggatcaatgagacttgcttaattcttgtcaagaatacaaagtcaacaatggctgagatgaaaatattttccataaagtaaaatgatcaaatttgcaaaagcttggttacatgatgtgaatttcactcaaaccacaaggcattacactagagtcttgctgaaatttaagacagactgtcttaggaattatttgccaatagacaaaatgaatatggggtggttgatgtgctgaagtggccaatgagcatgctttgattgacagatgaggaaatgtatccctacattttatcaaactatgtggtgtctgatttatatcactgaggtggccattcattatggtcatgaactgtgaggtcaaagcacttgcaaaaacttgccaaaatacttcctattccagtactaaatatttctcatatttgtttgcctagaataacaagatgcaggcttggtagtcctttcaagaatgtgaatttctttggtatgtctgagtttagtaatgttgaattgtgtgtgtgtgtgtattattcttaagactttgcctgtattttaacattttatttggtaaaaatagcattattttggcaagatttttaaagccttgaaatgggacaaattaaagtataatgctttttcttgagaaataatttattgctccatgttgttgaatcataccattagtcatgaaaccaaaaccctatatttaaaaagtaacaatctcatattcaaagtatctcctttgaacatttcaaagaatcttaagcaaatctttatgtgacattaaattgtacattttaccttgttattttagatctatgggtggagttcattcttcgggctaagtgtaggtatgaagtgtgaatttctgtgtcttggtatgccagtaacttagggcaaggttagtagagtggcttttgtgagaaaaaaagagctgttttatcagacatcagttggaactttgccttgttttgtttccctgaaccgtccaatctgcttcatatacttgtgtgaaatcctaaaatgtctgtatgtcaataatttttagttagcatattttttccaaacagacatccttgtgaacatcagccgtttctgagaactgggcccacggaccatagctactttgagtctcagtctatgtaatagttggggtaaagtaagctttaaagagagaactcacaacacaatggctcaagcaagacacaagttgatttctttctgaagctgccatcctgggtatgtgggtatttggatgagctgggccccatgaagtcatcccaaagtgcaggcctctgtggcatctctgccaaactcaacacttactctccaagtctgtccttggcttctctatttctagctggtggaagggaagggaaaaatgagtccagggaaggagtttttgtagatggcttgggagttgcatacatcaggaagaactttgtcacaaggccacaactccttgcaggaaattctaggaaacatattccctcactgagcagccatgcgcccaggaagaagagaagaatgagtctagagtcgcaagcagcaatatctactacaggcttaaataaccaaaggacaaaatgcccagtccattataaatatattccaaaaatgaagccatgaaatttgacttcatttattgaagtcaaaacaaatttattgttatgaactaatttctctattttatatttctcaaatgattacatttttaacacagaacaaagccatagttagaatttcagtaatcgtatggaatatccaaaaggtacacccaatacacccaaaagctaaaccagagtcactgctaaattttatattgttctgatttacctaacagattagtgaaaataattaaaatcttgctgctttttaaattttaaatatcggattttggttagaactctaagaatatttaaaactatctttacctactcttgttggacaatcatttgaggagctaagagtgcagagaaggcagagaaatgttctcctcaacccaagcaaaagcacagaagaactattgctaatccaaaatgcaggaaggaaatttcacccctcctcccttcccttttcttgggctagtctgtttaactttaaaagcagaatttttgctctcacaaagtataccagacaggaatttgttcaaattcctcaaaaccaagccaagaaaagtttcagaaagttcagacatgcatggcagagtggaaagaacatagtctgtggaggcgagcacggattcattggaatgccgctccagacatccactctgggtgaactcagggatgtgtttagtctcattaagccacagtcttttcatttttaatgtaagagtgataagaacgacttgccagtgtgttggtggagaactgaaatagagaaaggcctggtgtgtaacaggatgtttctctccatctgtgtaatctgtgacagtcctaggaagttcaaagttatgactgttctgggtttgtggccaacagttgtctgtggcttccatgggttggagctgtctaatggggcccagnccaccatcgctctgctcagaagctaatcataccaaaacatgaggtttttctccaagcgacaaagaggggctaagcacccaaggatctccataatagtagcaggaaaatgaagctgagaccttaccctgacttagaagttcataacaataatggccccaaacaagatgctcatgtctattggttgtggttaataatcatgcaacaacatgaacttcaaacccagcctatgcttggctcttccagggactgaaggcatctcagagccccaccccttacctcagaaaccgcaggaaggagcctgggttctcagagtccaagaaaatgcacctgataatagcacctgaacttcctccacacaaagaaagcctgatagggacaggggattaagcttgcatggagggcaggatccaacccacagtagcacttccttgccatgcgaccttagcaaatggcttaactcgctttctgtatcagtaaaatggtgtttgtagtactaaggtgtcagtgaggattaaatgagccaagcctgacccacagcaattcatgacaagtctcagccactttaatgaatgtgatgatattctaacgtttgggatgctgaggtggaggacgatggtgtcccacctcctgcaaatgccatgtgaacactgaagctacacaagcaatggctctaaagtcaaagccagcatcaggactggtataaaagaatcaccaggacaagggcagggcagaggggaggccaaagcagctttgtcagtgcctgcacggctggacctagaggaatttctcgacatagagggtaaaacaacccattggcccacatttctgaggaccgacgtgggagaagggtcacatgaggccatgaaatgggcatctgcagaggaaatggaagccagtgtctggagacaagtggagccattgagctagcaatgcgtgatagatactgtgggaagggatgtggggaatgcagcacctctcaagggaggtggctgagtctggcaatgagggcctggggcatccttagtcagcaaagcaggtctgcagcctcgggagctctggcagctggtgtgggtcaccaactggcctcgggcaccattcatggaaattaatctggatagtcccctcctggagaccctccaggatggcagatgagcagactggccggctttcaaagtctgctgacctctataattgtggctcttcacatggtccaacacactcttcccttttacatgtgtttcctctttgaatatttccaccagcccctctccctgacctctctctgctttgttactttcctttgcccatttaatctacattttatgttttgtccagattaacatacctgtgtttcatctgccaggcactggcccaggccctaggaatatggtggtgaccaacacagagtggctgctttcatggagtttacaatctacaaagggacaaaaaggataaacaactttaaactttaaaatttggacaaacttttataaatactgcatttttccaataaaatgagataaacaatggtataacctacattgtacagaatctgtgtctccctaaaattaaagctttgagataaaaggagacaagaggtattgttggacttgaatttgtcgtgcaaataagattaaaaccttgaatattagccttttgattgttgtctctttttgcagtatgaggtagttgtgaattctaggtcaaaacataagggttaaaaggctggaaaaattaaataggccaaatgaaagaattgtcttgctaaaatgtctatgtagatgataaaatgtccttgtgcctctttaacttcaatgatttgtgctggatattttataagcgtccattaggcaatagcaaatttattcaactataagagaattacgaaacacaactatcataggagatacttatttccttccaaccaataagtaaccaccagtgatgagaaggcagaaagtgacaagattaaccaagtaagagagttggatacaacattcaactccatttcttcatgtcccaataagagacaacgtttgacctgccctctcattcctaatcttgttgaatgaatctagtacagaggttcataaaaaccgtcaggtatataatgccggattttgtcaacaaacaataacaataattttttctttaaaaattattccaggccaggccgggtgcggtggctcatgcctgtaatcccagcactttgggaggctgaggcgggcggatcacgaggtcaggagatcaagaccagtctggccaacatggtgaaacccccatctctactaaaaatacaaaaatcagctgggcgtggtggcagggacctgtagtcgcagctactcgggaggctgaggcaggagaatcactttaacacgggaggcggagattgcagngagctgagatccagccaccgcaccctagcctggcaacagagcaagattccgtctcaaaaaaaaaaaaaaaagaaaagaaaaaaattattccaggccaggtgtggtggctcacacctgtaatcccaacactttnggtggccaaggtgggtggaccgcttgagctcaggacttcaaggccagcctgagcaacatggcgaaacctggtctctacaagaaatacaaaaattaaccaggcgtggtagtgagtgcctgtagtcccagctcctcatccgcctgtaatcccaggcggagattgaagtgagccgagatggcaccactgcattccatcctgggtgacagagccaaaccctgtctcaaaaaataacaaaatacacaccagggcctgtcagggggtgggagggaaggggagggagagcatgaggacaaatacctagtgcatgtggggcttaaaacctagatgatgggtcaataggtgcagcaaaccatcatgccacatgcatacctatgtaacaaacctgcacattctgcacatgtatcccagaacttaaatttaaaaataaaataaaaataataacataataaaataacaaaaaagtctttacatctgaaataagtgtacttaaaattaaaaataaaattaaagtacaaaaacttaaaataaaaatcattttagatatttcctcagatttaaatggaggtcattgaagctcaaacacctggctgcaaagtgatttgtaagacgaacacctacactctacaaaaagacttatgaataccctgcattggttgatgaacaagaacagtgagttataccttctgaatagtgaattaaataaaatactattaaagttttaaataattgccaattgctccatatttctctgcatgttagagtttagctagtgtttccagggatgatttttctgtattcctcccagtcaggaatagccagtcagtattactccatcagccacagcacaaatgtctgattgctctcttgaggtcgttagcccagatacttgctagaaaggtatgattgctggccaaatgagctctggcctggtgcagccgtcttgccaatgtgcctaatgagagcgcggaaagacattttattccaggcataagcaggccagatcacagctgcccaaagctctctggctttgattaagtttgttgtgctattatgtaatgggtacttgaaccatattccgtgaatgaccgttttccttcctctttcgccactattctaagtatgttgtctgtatctaatgttgccttgcagttagttgttaaaatcaagcagaaaagaaaacaggcaaatattaaaacgtaaatagaaaaagttgttggtggggaaaatgacttataatgcattattctgacataatgtcttggggtccaccaggagaatcgccccatcacaattcagcctctctctgtgctaatgacggccttttcatcctgtgctgttagtgatgcataagccaaattgtgaccaggcagactagaccacatgtaagcagagaaccacgtctttgtagacattaatttaaaactcgttctaatcaaatatgttcatttatctgtactatttttgatctatatcttaaatcaaaacgaagcttttttgacttctggtttctgtttctgcttgtaaagagcttggaagttatcaatcctgtcttcataacaagaaaaatgctgagcacactgaaagtcagtaactggaaatcaacagtgttttaaaagttaattggttacaattttctcatattcttctctcacctctgtagagttctggtctacattggaaatgctggaaaagtaggaatatacagtacagagctttcttggcagcataacttaatacatgaaaaatgatttttaattttagtttaacctttaaactggaatctgtagcacattggaaaaaaaaaacctaaaccagttgaacaaatttcaattttaaacatgttttcaggctttggaagcttactgaagaatgtggatagcaaatacaggaaaaattctgtgaatatttttattcacatatgtaaacgtgaagttataagacagaacaatttccccatgtacaagcttcaaatttttagcagttagttaacattttctattcagttttaaccattaagttgtaatattattacaaatgatattagaacacatctttgcttttagtgctgtattataaatatttaaattaatgacattcagtgtaaatttggtatttatcgtaatacaaataagaactttatttttagttaattccatgtgttatttgtatttcttatctcacatttatttaaaattacatttggtccctatcactcaggtatgtttcttccctcccacgtagacctaatcggattttttaaaattggattttaatctgcctttataaacaaacgtcaattatttatttccaatcttggttgatctttttattccgagattatttactagttcattcagccttcctattaaaaataaataaataaataaataattgctcttgtagctcttctgaggtagactgaccaggtctaccttattgttaatttttgcattttcatttttattgaatatgtatgatgatgcagatatgaaccatgtgcatatctcagaataggaggaaaaatatcttagtacttttgccaaatgcttggttctaatttttggactattatgaattttttatgtcataactattatcttaacattatgcgaacttctagcccttttgcttcaatttcctttgaaaagatttccttccaataaatctttaaaatatgggtgacaaaactaaaagaccctcagaggaaacatatttctggtatgacatattttgtgtactctttaggacaattgaaaagcttctaattaacatttctttagcgccttagtggtgatcagcagtatcatcactgccctagtgcatccatggaagttgaggtccagggtgtactgactcacacaggttacctaggtaagaagtaatggatgactggaatgttaggaactgtaaatgacagctaactgtggcaaagcgtagggttttgtttttttttttcttgtcttgtcttttttttttgagacagagtcttgctctgtccccgaggctggagtgcggtggtgtgatctcggctcactgcaccctctgcctcccaggttcaagcaattctcctgcctcagcctcccaagtagctgggattacaggtacatgccaccaggcctggataatttttgtatttttagtagagacaaggtttcaccatgttggccaggttggtctcaagctcctgacctcatgatccacctgccttggcctcccgaagtgctggaattacaggcatgagccactgcgcccggcctttcttttcttcttaagacaactaatggaaaataaacttccaagaagctcttttgacctgacctcactgctcctcaaaggcagaatgctgattcacaggctgaggggtatctgaccaggtttctttgctacccttactctcagcctacctggaaattgccgagcgcagggggcgcagggagtgctgtagttggcgtgtggactgagcgtcctgggaactgttctgctgtagtacctacactttcctgccaatgtcagcatccggcttgtcaacacttcttttccatttgtgaatactatagtgaaagccttcacctctcctttctttgaagtattctctgttcagaacttggaactttagaaattttctacatttaaggaaacatatttcaactatgattctttattacttataaaaaattaacttagaagatatatgtaaacatgtgtatggtttaaggtaacaaagtgagtttaccaatatggcaagttgtgccaaaagagtgcttaagtgtaaaaaacaaacaaagaaaccaaaatcaaaattaaagtagctcaaaaacataaagtttataaatatttttaaggcatttataattttaaaaaagtccttttatgtttgtcccatgttctggaatagcaaataaaaaatgtggtttttgctttattttgttttattgccttgatatgccacgtggcaagcctcaggctgggtgtgtggagcacatgacttgagtgtgtggacaagttgtgttgaagattattattacaggcacaatgttacagcacagtctatcagggaaaaatacttaagtttatttttttgaggctgagcgtggtggcttatgcctgtaatcccagctccttgggaggctgaggcagaaaattggttgaacccaggaggtgaaggctgcagtgagccaagactgtaccactgcactccagcctgggtgacagggcgagactccatctcagaaaaagaaaaaaaggaaaggaagaaagaaagagagagaaaaaaaagagaaagaaaggaaggaaggagggaaggaaggaaggaaggaaggaaggaaggaaggaaggaaggaaggaaggaaggaaagaaagaaagaagaaaaagaaaaagaaggaaggaaggaaaagaagaggaagaagaagaaggagggaaggaaggaaggaggatcggagggagaaaacatggtttacagaggttttattcactaggatggtaacatcctcccaagtaatttagttctagctgctttatcgtttagtaaatctctgcaattcaggatgcacctttcttgctgttgctggtctgctgatccacatgatcttatttcatgtatgtggatcatccagccaccgtgacaaatgttcattatgttgagtccagatgtccaccttaagagtacacacccctcgctgctgctttttttttttaattatgtcacactaaatacatttactttatgataactactgttggggtacaatttactcttaccacagaaccagctctcagttaagaagaaagtgctgagatggtgtggggaaatttggtgaagaaaaacattatagtaagttacagtcaatctcactactgacctttggctaaaattttttttaatatgtggctaaaatctgcataacagatggtatatctgagtgaagagattattagaggatgagagaaactgagagttcaccaaaaataaaaactaataattgcaatatagattttgccatcaaaagtgacctcaaatgcaagtttgtagctgaattgaagaggtggccactttgttcttaatctggtggctgctgcttaggtttcattgtagaggctactgagctgttttgcaagtttgtattatctgtctcatgatccagacagaaccagagtctaccaacgtggattaagaattagtctgttttcttctgtgtttttctatgaatatactgaatgtctcatcttgattcagaagaaaaacctgaaagcttacccaaaaacaaacaaacaaacaaacaaacaaacaaaaaaaaccctgtgtgtggtttgtcttgggacagttcagaaggcagtcatttaatttcgtttgggaacattttgcactatttatttcattcaaaatatgttaatttattttcgattttaattaatgtacttttgtaatggcttattccgcaagcagagatagcctcccttcccccaaattctaagatatgcatgtatacagtcatcttactacatagcaggaattttaaaaagaaataagcttttctttccagtgcctagttgttaaaacagcaaaatgataaaaatttatgtaatggtactattgagctgtctactgtgtggcaaatactttatacaaataagcaatgtcttaggatctaggcttttagccttcaaaaatctttaaagaagaatgaaatgtcatttttaagtcttgtatttgatctcaagtgaagagattcaattcatttcaaaaatacttaatataaattatatgctagaccctgtgctaggcactgcagggcaaaagctgaagaatcagagaggctgtcaagttagaaaagtgtttctcaattggcaatttttccaacaagggccactggtgaatgtctacaagtatttttggttgggggttgcagggactgttggaatttagtgggtagaggtcaggggtgtggctacacatcttgcaatgcataggacagaccttcccattaaagcatcatccacctgcagatgtcactagtaccaaacttacattttgtgtagcttcagcaagttctctcttaaactattcatcgctctcttttttcattagttctgcagtttaataagaatattgccctatggagcagttctagcactttgggaggccgaggtgagtggatcacctgaggtcaggagttcgaggccagcctggccaacatgacgaaaccctgtctctactaaaaatacaaaacttagccaggcgtggtggcatgtgcctgtagtcccagctacttggggaggctgaggcaggagaatcacttgaatctggaggcagaggttgcagtgcgttgagatcaagccactgcactccagcctgggtgacagagcaagacaccgtctcnnaaaaaaaaaaaatagctggagattaggcaagttgtatatatgtaaacataaaaagacagaaacaaataaaaaccaatatagcacaacccaccccatcttgaacaagttgtacttctaatcatgcacatgtgaaaacatcaagctcttatttgaccaccattcaggattgaatatttagtcattgcaaatgtagttgagcaaataagtatctatttggagtttaggattaggatcatacacttccacagttaaatctactgactgacagtagacagttcattgagacacagatacagcatagtcatccatgtatagtggttgagtaacgtgtaagtttcttttcccttccaactgaaggggatgctcaaactgagatttaaccaggacaaagtcaatcagcttctctctgaaaagggtgatgtctgatcagagtcagctggaggatcagaaagagtcatttagaaaagtctgagggaaactgatgatcttgagtaacaagaaagttgccattttgttcaccacagatgaaagtctattcccagacagtaaatgaagccggataatcaggccaatttcataaacagtagatcagattggaagagttaaaggaccagataagaaaacaagcacctgtagcaggatgacccgagtaacacaaaggatgattcacgcaaagggggtgcaaggactaattctgcagactcctatagcttgttgtgccttgcaagcccaagagaggttcccaaagggactgaagcctggcagcatccagtgaatgggctctagcatacctggtaaggagctgtctcagaagatggggtgttccatatccctggaggggttaaagttcaagaacattgctgaggatccaaacactgggagagagaaagaaatggatggtcataaaataaatcctctttgttcttttaaaaattctgagtgtgggagtgaaggtgatttttcagaccaagcccttgggtactgactcattcttattccactgtctcgggcttacatctccaattctaacttcccaacttctgtcatggcagatcaggtattaccattgttagaatagtactataggtattttgtgtttggagagaggtttagtggtaggaccccaggtttgtagtgtgttgctagttatcatcacaattttatgaccctctcacttgagagtggaatgacaatgtggttatgtaacctaagactagaagaaacaaatgggagtactcccaaaacacaaatatctcacctcttttttcatgggaaaagagtcagcagaagagggcaaagcggtaaataaaatcctaaaggtaatcaaaacagatgggaatataattttgttcagagctataaaaaatatcctctactattctcaatacaggcagtgtgtagaaagtttcaaggttagtatttatcttctaaacatagacttgtggtcttcttatttaatcaattcaaaccaccagtggaagaggtcagaaagaatgaatgaaatcaggagaactgattatcatgagtatcattgttgtgattgcatttcttgcacagctttaatttgtgccttgagagtttggggtctgagaaagtagtgctattccaagagctctccagggatatttttggaacaggccttattgttctctaaaaaattaatgcgttatattcccaggtgctgttgtagcatcatttttcatttaacaaaaatatggtcgtttagccagaaattttagttctataattaaaatggactaaaaatattctgtagcacatctagaaaaaatattttaaatgtatcaatgttcttcctagaaattttccatgttggatcaaacagtagatgaagacttttctgtgtgtatagcatgggtttttatgtttatatatgagtgtgcaggtgttttaagagaattttgaaaatcagcaaagcaaagtcaattcactacaaaatagtttccaaattatctgtgcagtattaatattgaggaagaattaatcaaaaagcaaatatttgagaaatgctttcctatgtctgtaggctacaaaatcattgtttaatttatcaacaatctttgagtgctcctcagtgtctggtacagaaatgaaagatacagttttaactttagagtgctcacacctactgagggaaacaaacaaggtagagaattcagtatgacgagtgacatgagacagttgaatgggaggctctctgggagtactaagggccattgcctagtgtagctgagggcacagaggagctgagctgggcagcccaggagtctgtcaagagggccagagggaagcagtgcagggccagcagcgagagcgggactgtgcaaaagaagtttcagtagctacagtttctttctcagggtcctccacttcccaaagaaaatgaaaggaacatacatttgatttttgtcagtttaaatattggtcgaccattggaaaaccatttttgaaaagtgatttcttttgctatgtttggcaaagccaggagtagtaaagagatgaggaaggaccatcttcgtccattgcctttgcctttgtaactgttacttcttcacagcctttctacccagtgtagttcacgtatgggaaacatttctttccatcgtcagcgtcttcctggggagtgttttggttatactttcacgaatgtctcacctctttgtgtcaacaaacccttctctttgagctgtactgtgggtttctgtttctttcctttttntcctttgtccaattagaaaaaaaaatggagtagaagtttcaaggctactagttcaatgtgctcttttttacttgactccaaatgacatcttgcttgcagatgtgattaagaaagatctggacagggagagggaggataattttcaatgttcctctcaaatttaatcttatagtgacaagctccatattctacatgaaaatgctcagttaatacagctattaaacttgaatggtagataatagtggatttctgtagccactgtttggtttcattcaaaaacctagaggaaaataagggatatacattataaattgtttgtttaatgttaaaaatatgtttcatattcagtgcacataaagcaaaggtattgtccagtgttagtacctatgaatcatcaataaaaacagttgcttctgtttgctgttgacattttatgtatgtgttgttcctctctgaacctttttcttaaaagtcaacttttaagaaacttataagtttactgtggagagcctatgaacagatgcatgagggggcgcctgttcatatggataagatagggctataaatgcccttatcttgccagggctcttctaggcctctttagggttaaggcatactcccttctgagaatttgtgttgtaaccggttgtctagcttcacgtcctgtttctatggattgtttgtaaccagcttttgctgcaactgttactgctgattaataccttgctaatcataggttatggaaagactgttttctgttttaaggctctgttagaaattgctgatgcacacaatattgtaaattcttatctctgtatactgtacttctgcatacagatattatgttaaagaattacttcatccccatgtgaccatctcacttcataatcaaatgactctaaatccctcactaaactacccccaccctcactaaacttaacaataaatgctggtatatccagtacgttggcggcatcacaggaccagaaggcagtgatccccctggacccagctttcactaaaaaaaaaaaaaaaaaaaaaaagaaacaataaacttagccattataatccctcagaaaaacattaactatatttaaggataataagagaaaataatctctttttaacatacttattgatacataataattgtacatatttatggggtacctgtgatattttgttttatggataaaatgtgtaattatcaaatcaagatatttaggatatccataacctcaagcatttatcatttctatgtgttgggaacatttcaaggcctctctgctagctattttgaaatacacaatacattgttgttaactataatcaccctcgtctgccatggaacattacaacttattccttctatctaactctatgtttgtacccattaataatttctttttaaagccccagtctcaatcaagagaaatataaatagcatggtaaaaaaaaattttttttgacctgatgatatgggttcaatttctggttctaccactttccaccttggagaccttcattaaccagtgaccttcacttttcttttctgtaagatggtattgataatatctgccctcatccccacttcgccatttgcctagctaataagacagagatttccttagcatgttgtgttaaagaggctagaaactcaacatgctttacaaataccagctgtatgacaaggagttacagtaccaatgtacaaaatatgatttttaaaatgaaaggcaggacatttatgaagttatattctatagaaagtatattagcacacattaatgcataataggcgataggactgcactttgcttctttctcttagcttttctttccagcctctttttctcccttgtcatcaagtctgcggctggacttgccttctgttctgcatgccttggtgtttgctgttgctctgattctgctttctcatgcacaggctcaaagaaacagtgaggctgccccaacctgtgcaactctcctctttgtttggatacttggtcatatgcacattctagtttaaatttctgtgtaactaaaacatgtatgagtggagattggttttatattctgtaataataaaacttatttatagaagtagaatttattgaggtacaaatgtttaccagttttatgaataacaacttatatttattttataaacaacttcaatggtatatgtgtgtgtgttttctcacaaaagcatattgagcaattattttcaattttatagtcataaatgatacctgaaatatcttaagaacctaccctgtgatttacaggaattaagtcattaaatccttacaacacagtaaagtctgtgttatttcccattttacagatggagaaactaactacagaaagactattaaagaggcaagatatcatcacagttggatgtgtggggctttgtaccagaatgactgagttaattccagttcccttaattattatctttgcatcctcagacaagtcactttatctttatatgctttagtttccctgtctgtaaaacatcttatagtgttgttaggattaaacacatcttttccctacatggcattttaggttctcttaaccaatgttaaaaaatctatccacatatttttagctttcctttctttccattactagaagtactttctatgaggtaattctcccagaaaatacaaagtcctatttggtgattcactgccttacattctggtgttcggtaacaccatggaatccaaagacaagctccatcatatcaattgtggagcctgagatactttactctccctgtgagaaagtgatttcaaatgaaactcagttctcccatttatttcattttggttaaaactatagcaggtgcttttcagcttattgaatgtgttaggctccaagtggcaagcttaaatggtacatatcctttttttaattatacatatatatacttggtttgactgtttatttccttttccacattattatttgcatttaacagctaaaggtataatatcaaattattttattgttagatgttgaaattaccctggcttgaagtcagaaacaactttattaaaattttaagagaacagtaaaatgtgttgcatgtgattctaatcaatttttgaagcacttttgtcattgagaaaataagtatggataaatattgattgtttttctggaaaatgctttcttatttcaaaagtcagctgtgactcagtctgtcctcctctgtcatgacaggaactagtatattcaccgtctatgaggccgcctcacaggaaggctgggtgttcctcatgtacagagcaattgacagctttccccgttggcgttcctacttctatttcatcactctcattttcttcctcgcctggcttgtgaaggtactgcgaagaattgtactaagaaagtctatgttctgtgatagttctgcagatcttttgtttggttttggttgccctctgctttgtcattcttagactttggtaaatgtggtttgtgatttttttcagaacgtgtttattgctgttatcattgaaacatttgcagaaatcagagtacagtttcaacaaatgtggggatcgagaagcagcactacctcaacagccaccacccaggtacgctatcagcaagaggatgttataacccattcgagcatgcacatgtctcaccctcaccatcagtggaccttggggaagctcttgcatgatgggcttctatagtctgactcaggaagctgtttaaaaatacagttgccagtcaggcacagtggctcatgcttgtaattccagcactttgggaggctgagggagagggattgcttgagcccaggagtttgagatcagcctaggtaatgtagcaagaccctgtctctacaaaaaaattttaaaaattagccaagagtagtggcatgagcttgtagtcccagctactcagtaggctgagatgggaggatcacttgagctcaggaggtcaaaggtacagtgagctgcaatcacaacactgcaccccagcctgggtgacagagcaagatcctatccccaaaaaacataaaagaaaataaaataaaacaggttcaaaggcccaaacacagaacaatcaaatcagagcttccaggcagtgaggtaatgacacctatattttgtaacagctccgtgggtggttttgatacaaaaccaagatcgtcaaccacatttattacatacctttgattctgacatcacaaatcctcaaatggcttgtggataaaatgcaaccactatggaatttcttacttttatgggtatttattcatgtacttgcaagcctatgtaatgtctatgtgtaggattagaattttgtaatggaaaatatatttcattcagaacaaaccacagtacaataaactatgaaaaagatccctcaaccccaaatgcatgtttgtgattttttaaagaatccctatatattcagaaggattcccataagttccatagcaatattgtttccataaaaccataaggctcctgagaatttgaaggtgggctacagatttgctaaaagcagcagggcagttattacaaaagagggcaattcagatccaatgcctttgtttttatttttggttttgtttcacgtgtgtgtttttgtggtgttaaaaaaaggaagggcagatttgtgtatcttcttctagcgtgaatctttataaaagagagagaaaatataaattccagaatcaggaataaaatgatataatgccttgtaattattataatattaaattctaaaggatatttaacaactttgtaaacacttacaaatactgttaagacaaaggtatctatcaaggagccgaggtgtacaaattttacttttgaaaaccctcaagttgagcttttaataattatttgttaaaatgcaattaaacacactaatttgggaaaagcctggtggtgttgaggcatctttatgatgctgtaaaagtctgaattgtccagtcaattcattatagatgaaatagtgtcagtcttattcagtttttacaaacaactgttcaaacttccatgaaaggttttttgaatatgctatccaggttatatcagaaatgccatgtagaaaagccttcaagaaatcaaccatagcaagttctcatctcctctctcctctctttttgcaaactatgagagataagaaagttcccggtccattccgaggtggcagcctctagaaggacatgttttcagttatgactttgagacctaaatggaatatggaactatgatctcagcccttaactcacatgcagtgttcctggagatgacctaagattcctttaaaaccatagtatgtttatggagtaactctcacgtataaaatcacagctggaaaaaatagtgtttggaagcaccaccaagtcaggcaatggttctaatagaaagggaaatgtgaagagacttggaacaaagttgtacagaatcattttgatgcaaaattgtacttaatcaagaggaataaattggtaattcagaggtaatgcgtctagatattcagcagtggtaatcatacagagaattgtgaaaaaccttcattaagaaaaatgctatagctgaggacctggttgctaagcaaccaaagctcctgctgtcagcctctgggtgaaacaccactcaggcagcctagcaacgagcttcaggccccgcccaccctggctagccacttgcgagtgggccctctgatgcttttatgtgtagagaagggagagacataatttgccatgatgctattatgtttgtaaaatatgaccaaattccgtgaacagggacttctcattcagtggtttaaaacactagatcacaagttactaatttgaaggaaataaaataaagatgattattataatgcacacagcttttcaaacatggaagtacattcttgcctttctctagcatatggaaaatttgcagttgttaaatataggctattctcatcaaaggactaaagggtcctgggcttgttggacttgttgatgtctaagtctgcagacttcccataacagccatgtgtatagaaaatatgtgnnnnnnnnnnnnnnnntatatatatatatatatatatatatgcacattttcagaacaaagaaggggctttctgttatattaggcagctcactaggaactggctctgacagggaccatttcaagggaggcgaaaggaaatctgtgatgagaactgtttctttaaaataacgaagcaatatttttcaattgaatggtcaccaccaaatgcctccaagacacagcataggaacaaattacagagacattaatgccaagggtctttaggagaccataatataacatgtaactggaaaacagaaggaaaatgatgtagaaaatttcaaattactaaatattgactctggcctctgagagccttgtgatttcatacctgcctgattcttagacgtgggtaactattgccttgtgctcagttgtggattgggttgaaggataagtgattcatacacccatgatataataccctgctttgttaatccactatagggttacccaggccacaactttttagtgatgtcccatgactggatgttgggatgtgcaggtgagctgaaagacgaagtcctgagtaccgtatctcactaacccagacctttactcccattatttagatgctttacttcagatacctgttatgagccccatggtcctctgatctagactagctcatgatggagatgagcttatcccaactttgccttgagtagaactcaaagaactgctaaattttagagccacacaaggctacttttctgttaaaatggtaggagacagaaagcaggcttctgacagtcaatacatatctaacttttataaatgaatcataatccaataggatcatattgaaggtttggtaaataattaaatattctgattgcacaaagacagcaatcatttctgggtctgttattatctctgattgagcactcttgctgacatgtgttttctgaattaatatcccctaattaacagagaagactgataaaggtagaactggtgtgggaagaatgggcacttttgagtttttctcctttgggtttccatcaacattcttagaatatgggttatacaatgcctaaagcaacttgtgcataaatgttcagtgctgggttaaaaagtagagaaaaaatggatgaaaagacacatgatccaaaatggaaagaatttctcctccaaaaagaaagctacatataacttaatgctcaaagaaaatactttagcaaaaatgtgtttactgtgttccaagaccatttatttcctcatgaatatacatcattaaaatatgtgagaaaacaattcaattaatgcatttcagggaagggtgcttctagcatggtgtaataatgagtgccttctaataagaaaccataataatagaatagtacaatcatcataggaaaccataaatgagaattaaattttgtgaggttgcatcattgaatatattatttcgtaatgaaatcaagccaggtatccactttcactattttctgtcatagtgactattagctattttttgaaaaaccataaattttcaaagaatccaaagagcatgcaaattgcaaagtaaattttagattttctctgcattactgtgtatgtggatgtgtgtgtgcctgtcttgagtttggaattctaattcagctgctagtgattctccattaccaagattctttctcttaccactatccattagataacctgttattcatgttgttgatgatgatgatattatgcctataatggaacaatcccagaagcatttttagctactttacaaaccaaccgaccagttataaaactgtagcagttgcaaatggacaggcctgagagaattctcaaagttgaaatctccatgcgcttccaaatgattgtgctaagaggagagtgccatggaaattcatttgtgttattggtgagattctactaattcagaacatcttcttacagtcactcccatatattgagatctgcactagtctaatggctttacaaactgtcttatttaattttcataataatactgtgaagtgggcactattaatgtctccactttgcatgtaaaaaatgacaatgattggaaagttaattatctaacccaatgttatataactggctagtggtaaaactgggactcaaaaccaggttgctcagctgcaagagcctttttattcagcatgagatgctaccacctttcaatttagccattacaagccctgctgtcaacatgctgtctttgttctggcctcatgttaacttttcttttcttaaaaaagacagtatttattgctggctgaaagggacattcgcatacattgttgccaagagtatgaattaaaacatcttttccagaaaaccgtttggcaatatgtatccaaaagtaaaacatacatactatctgaccaagaaagtccgcttttagaaatgtatcctaagaaaaattgggggcagtagccaagggcatatgtttgaggatgtttatgaggcattatggcaattttttaaaaatggaaacaatcttagtctctagcaataagttatcgattataaattatgaaattggaataaatgcaaaccattaaaataagaattgaactttattgatgtgaagatatgctgatgacatattagtacaaaaaaagatcctatgtttgtctgtgggatgcaggtgtgtgtgtgtgtgtgtgtgtgtgtgtgtgtgtataatacctggaatgttaactgtaaaatgttagtggtggtgtctctggggaatgggtttaagctgnttcttttgcaactcatncctcattttctaagcctttaatagtaaaatnatttaattatagaatgagaaaggaaaattacatcagtnnnntnnnaaaaannaaattaatggacattctaacagaaaccacgtatactagttaagttacattcagttgcaagggacaaaaaagcccaaaataagttgcttaggtgacgtatgcgatgaggaatcccattctctctcatataaagttggaggtgctcattcagcatggtatggttgtctgtgttgccacggatccagacaccttccttcctgttccactttgcacagcccccattaccacagtcaccttgtagactagcatggctgctgtagctctaaccctcacatccatatcccagagagcaggaagaagaaggaaatacaggaaaagagggtttctgcttgcatcctatagtcctgtatgtagttacatggtcaagctcagcttcaaaaaaagggcaagaactggccttttttcttggtagcctagtggccccatacagattctctttgtaaaacagggaaaagagatacaaaggaaagactggcagtccctgccacgctccccgcaaggatttgagtctctgaggattcttggcttctgctagctagcccagcttctgtctcaaagatctactgtaatattaaccaatctaaatgttgtataattcaggggattaaagagaaattgaacgtttttgcatttcctttacttatttcatacaaataagtgaccttgggagaacacttttactgaaaattaaattgattcttttgattttggtctattgaaaaaaaaatttaagagagaaaattagtctgctactgatcaaagtcaatatatgccccagggatcctttatgccacaattagagagtataggatgtatcaacagagggtttttattaataactggtgactggcaatgctggttttgtcttttatccttaaaatgaggcttccagaattttgacagaaactttacaaggtcctataacaggtttgcatctccactgatgtctatattctcagaatgagctattcaggaagaatatgcttgattcaccccaaaaagagtttttgcagtttattctaaagaaaatcagaatcctgtaaatggacagctgagatgagcaatttgcagttggcatctttgtcaaaggagctttatgtaaatgtgcaccttaattttgtcatttaaaaatacatataggaggcagaagctgggggattgcctgagctcaggagttcaaaaccaccctggacaacaagctgaaaccctctgtctagtaaaaaaaaaaatacaaaaattagttgggcattgtggcgggtgcctgtagtcccagctactcgggaggctgagacaggaaaattgcttgaacccgggaggtggaggttggagtgagtggagatcacgcccctacactccagcctgggcgacagagcaggactctatctccccccaaaaataaataaataataaaaatacatatagggactgtgatttttggcaactgtctctcaccacctcactcattttatagaaggcaagtttattttccacaaaggatctaccaccactactaccagggatttgaaactctaattacaaatcctaggtaatgggatctttgcatcatctcattcgtatttccatcagcagttaccccttcgaatacagggtcagactaacaggaggacataaataagggtgacaaaggaaacatttatttctgtaaaacagcgacctcaggyaggaatgaaacagtgagcacttgtagttagtatcattatttagaaaaacaaaaatgtttaattttgccctattcctaccatacacacatacacacacactcgctcctaagaactagaaagaaaaatatgttagtgggaaaatacgtgaaagtgggaaatgtcttccagctgggtgtaatattgacacataggtaatatgtgatgatacctaggagataataggtacctaggagataataacctaggtatctaattcaatggatttcagatttagtgggcctgtagcatcttgaatttcatcacttgggtctttaatccaaaagaaactacttgttcccttcataatacgtacataaaatttctgctgagcaggttaattgctgcctagttaaaataattacacaaagtgtattaagcaatgtggcaatcttctaccattaaaataacaccagttctttccctatgccacacgcacacttcttattttatggatcctgagaatatttttattaggtcagtgctcctgttcagccttgagggtaagataccatggtagtccaagccagggagtggaagactgtccactaaataaaaataaaaatttgcaacctatagtaaaaacacaccagacacaatgcttttaaattctgattttcattttctcccagatctaatagatatttatcaatataatggggataaaatactaaagtatttctcttgtggagaagaatatttgagcaattatatgcaaagcatatgcaagacagaaagtgcctgacatagcaagtagtcaataaactcaagttattatcattgttcttttttcattcatttaagctttcaatgttaaagaactataattgaaatgacttcattccaaatataaatccataagaagacaaatttttgataacataaaaatatacaaagctggagacatagagttctcaatgttccatattaaatgtctctgactcatctcccttctttgtcttgaagatgtttcatgaagatgctgctggaggttggcagctggtagctgtggatgtcaacaagccccagggacgcgccccagcctgcctccaggtgcagtacaatgacatttttaaaaatcgcccagcaaaggtctttgaattttatttcatccaagaaaatccacagctctttaagctctagatttgtccaaatttaaaatcctgaagttagagatggtatttcactccttcctctattcccaggacctagcttttttttttttaacatacacaatagggatttgataagtttctgatggctgcaggcatgtaagagcatttcagtggtattgaatcaatgaagaattttgttgacatgtgaaatcttataaaaatattctttaccgaaggactgagttatgtggcagtgggcaaattcattgtttcatacctccccgagtaactgggaaaaatatgttaatacatagtctctctgtttttctgcatttggaagctttcagaggaacataatgtagaggtgtttctttagcaaagtgcactgatagcaaacataaggattgcaggtggggcctgagagtcctcatgagatagattctcacagtgattagaagatggagtctcacgtccctgcctgtgaactttctggaaaaaccatcttctccaagctgccattgacaacaatatggataacaataataacaataaggcccaataaactcctttatctcttcttcagggggccatactgacatcttctcttccttggtttcccctccttgccccctaaatatcagtaactcattcaaaataatgtcaccttaccaagagcagcacccctaactttccataatattttcactttcattttccctccaagcagcccactcgtaggaccggagaattgattcttccacctggagaattttattttctttagcctttttggttttcagtgacaaatcctcttctcgcaaggggtggtttccatagttgtttatatcctgccctcataatttggagaagtgttcacatctgccgtgggatgagactgtatctcttttctttcttttgggtctttctccagatagggacttcttatgcaactcaaggatgggtacatgaaaaataaaattgtactctgagccattactgtgggctatgtttatatggccattttaccatagagttatttacttctttttgtttctatttgtattgaggtgtgattaacaaataaaattgtaaatacttaaggtatacaatgtgatgatttgatatatgtacacagtgtaaaacgattgttgcaaccaagctaattaacacacccatcaccgcatatattcagtagttacctctgtgtgtgtggggtggggggtaacacttaagatctaatctcctagcaaatctcatgtatacaatacagtattattaaccattaatgaatcactttgtttattagatcctgagaacttggccatcttttgactgataactaatatctccccatttctaccccctgtccccagcccctggtaaccacaatcctactctctgcttcaatgaatttgactttttgagattccatatataaatgagattatgcggtgtttgtctttctgtgtctagttcatttcacttagcataacgtcctccaggcttatccatatagttgtcaatggcaggatttttttctttattatggctgagtaatatttcattgtatgtctataccacattttttttatccattcatccatcagtagacacttaggttgttccctgtcttgaccattgtgaataacgctgcagtaaacacggggctgcagatgtctctttgagatactgatttcatttcctttgggcatatatccagaagtgggattgtgggatcatatggtagttctatttttaattttttgggaaacctccatactgcgtttcagaatggctctaccaatttaaatccccaccaacagtgtaaaagggttatcttttctttatgcctttgccaaatagttatcttttgtctttttgatggtgaccattgtaacagatatgaggtgatatctcaatgtggtttaaatttgtatttccctgataattagggatcgtatgcactttttcatatacctattggccattcatatgtcttctttggaaaaaaatctactcagaatttgctcatttataatcagattatttgcattttgtgtttgtttttgttttttagtttgggggctttttgttattgagttgtatgagttctttgcatcttttgaatattatctccttttcagaaatatggtgttccaattttcccctgttccatagactgccttttcattttgtttattgttagttatttatttctgtcagtttaaaaaaatggagggcaataagtagaaaagcttacaatgaatattgataagtattattttgtggtcctccttccatcctatatttgatgttgactatgctgctaatataaaactactacaactacctttactcctactatcactactaatagtaataacaataatgacaaagattatagtactagcttcttacctaaaaagcacttttattgacattattttttaatccatcaactgatatttactgagtaccaactatgtgctagacactaagatagtcttgaaagaaacatgcacagtctccacactcatagagcttacagtatgcttgagagtgggagggaaaaaggcagcattgagctagcaatcacaagagtgatgtgggttttgaaagaagtaatgcaagatgcaatgagaatatataaattggggtccgctttgagaggtcagggaaggttttagtgcataatcttaaaatcaaaaccttgagtaattccagcattttgggaggctgaggcaggcagatcacctgaggtcatgagtttgagaccagcctggccaacatggtgaaaccccatctctactaaaaatccaaatattacccaggtgttgtggtgcacgcctgtaatcccagctacttgggaggctaaggcacaagaatcacttgaacccgggaggtggaggttgcagtgagctgagattgtgccactgcaatgtagcctgggcaacagagtgagactctgtctcaaaaaaaaaaaaaaagagagagagagaatgaggaaaagaatgactaaggatgaggtttactacaaccctgtgatgtagaaagacctacatgcctggtcagccatttataggagaaagtgctgattcagagaacatagggatgtgaattacattcaaccagagggagaacccaggactaatggctgggtcttatggctcctagccccatgctttatccagctgaaaaaaatatgcaagtaatcataagttcgtgatagagaaataaactatcagggctactctgagtgccaaatcagagtcgtatggtatccagagctttaaaatctattaaatccttgttaatagatgcctcaaggtggccatctgaaattaggaggcaagtcagcctccttatcacaaagatttgagaataacaagggattaatgacagggtgaagtgtgatgacaaaatcgagcaaggagatagtaaaataaagattggaatggtgtggggatagatgttgggcttgaagtgtatagatgtggcatctcagagacccctatatgagatatgtgttatgaatatagcagcatgcttatgacatcttatgaatgtgactcatatgcttagaaactggggggaaaggggtcaggtaattcagtgtgacacaagtttcattccactcaacttcatgtgtttggccctgaataaagctttccagaggcagcaatgaagagtgcatggaagaaaagtgggatggccaagaagttatctgtttgtaccgggcaggagctgtgattagagagagaatacatttctatgtctcagcaagaaaataattaattaattaaaagaaaagaattaagtaaaaatatattgaagataaaatggtcctgaaatacttgatggctttcactaatgattaatgccatctaaaactgggtaacaggaaaactttcatctcagtcttccactccctggcttggactaccatggtatcttaccctcaaggctgaacaggagcatataccacttctttttatttatttatttatttatttatttatttatttatttatttnnnnnntttatttatttattttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaatttttttgtatttttagtagagacggggtttcaccgttttagccgggatggtctcgatctcctgacctcgtgatccgcccgcctcggcctcccaaagtgctgggattacaggcgtgagccaccgcgcccggccctttttatttttaaaacaagcaaaatgtgaacatgttctcataaaaaattaatatggaaataaaattaaaaggaagagtttacttgaaatcttctttcaatgtatccttcctcattcttcttccaatgagtaacttggaggttaatgttgagtcgattcttctcagactcctgcatttatgcattttatctcacatgaaaaagcatttctgcctccaggaagcactgacattttcacagaatgtttcctcttcctacaccacttccttctcactcatccttcagaactcagctcaaacatcactttcttatcctcaaaaaataagccaggtacctctgttacatgttttcatatcatcctacacttttcttttatgtctgtcataattttaataaaataaattaaggatataattagttgttgatgtctgcttctccaaccaggggaaattccattactttagagaccataccagtcatggtcacttctgcaactgtgctttagacatgagagatgatcaattatgtttattaaccgagaacagtccagtgaaagaaggaggcagtaccttcttggcttatgaaagtacttatttgacctctcgcttagaagctgctctgtttgtcctcctggtcttgtctgtaagaactagaaattgaatagactgtggcaataacacccttttccatacagaacacttatttctatatgcatgtcgttggagtctaaagcaagcagtacacttggagtttgcttccagaaagtgcctcgcagaggttgtaggactttggatatctctggatagttttgccaggatgtctcctctaatatgtggtggcagagccttggcaggacataaccctgacttctgttatgtttcacctctttcagaactcttttcatcttgtaagcatataagataaatttgtcttatgtttagaaatatagtgttaggaatagcatcattgaaaagaacccaaactcattcttttttttttttttttttttttttgagacagagtctcactctcctgcccaggctggaatccagtggcacaatcttggctcattgcaacctccgcctcccaggttcaagtgattctcctgcctcagcctccagagtagctgggactacaggcatgcgccaccacgcctggctgatttttgtatttttagtagaaaccgggtttcaccatgttggtcaggctggtcttgaactcctgacctcatgatccgcccacctcggcctcccaaagtgctgggattacaggtgtgagccactgtgcccagctgaacccaaactaattgtatgttaatgagaaaacagagtttattggaatgaaacatttgaacggctgtactttgtaattttgaaactgttgaaataaattagaaaaaatgaggtatcaataatatctgagagacagcaaactgatgtttgagttcccatttaactactgggaactgttgattttgttcaaagtgggactttgtttttaaatttgacccaaagtttaaaaaccagagtattttacataaaaacccacacgtcctacccatcttgtaaaagagaatgatctggcaataaagagctctcatttccacctggctagagttggccagagctgagtggtggctgccacatgagcagaggcagagaggctttattgtgcctcagtttcccaactctccatattgtctccacagcactggaggtgagtgttttacctacccaagaattcatacatttatttttctaagagtagagaaagatttctctttgtccgtgactctatcgtgtggaaataaataaacaagtaataaattcataaatatataaatgtataaagatacacaaatgcatggatgaatgaataaagaataaatacaaggcattttaaaagcaccctgtgattacagaaaacaatgagaaatgaacctagtattttaagaaaaaagttattccgatgtgttaaatatgtaaccctaagcctagttttctcattatgttacttgctaggtctcaggaggcacttggttgggtgactcacggaataaatgaaaataatgtaactttagatcacaaagattatataataacaattagtccacacatttcagtgattccaaaatagtattttcatatattttatgtttttaacattttaaaatgttaaaatcataactcagcataattggaagctggctcaagttctcttataaatgaaatgtactaaaaaatcaaaactgtctccattggtattatgtattatacttttcaccatattttctactctgtatcctcatccagttactataagtaattaggtaatttaataaaacaagattgattaaggtggcttaattttcctcaccattattaaattaaatacatgaagtaaccaatgcttctgtgatgctgagttttttacgagtaataatttattctctctttctccctctctctctatgtgtgtgtatatatacacaaacatatatatacacacgtttttatatatacatatataagtgcatatgtgtgtatatgtatatgtgtatatacatatatatagaaacattgctggcataaaactattaggattttatatatttcaatgggcagtgattctgatcacacattagcaactaattttaaattgttttcctacattcccacatgtgcacatacatattttaccttatatcaaatatttccattattatggtatttgtttcctcttttcaaatattgcttatggagatagtcaatgaattagggcacataaaagatcttcttactcttctacagaaaaaataggctctattgagccatttgtggaggagaatataggcatcttttgtctgcagtttgaaaccacaaatttattttgtgttctgaaacacaaattacaattacaatcatagctataattataattagaataggaaccatatgcaggcatgcaaggaaacgattcaatctggtgcccttgaagtgatctttctgtttttataataaatgaaaatgcacatacttgtaaatttaactgcaagaccacaagtttggagcaggaggactagcagaaaggtattgttagcttggtaagttatatgtggtgagctctttaacgccacttgttaaaattttttgttaatcaacttaattatattaatttgtctaggacatttctcatgtttatccagttattaatgaaatttataccatgcttaataaggctgagtttaataagatactgagcatggtaaatgtgtgcaattcctaataaatttatcctcacgttaaaacgagtaagcgaatgtcctagaacccctgaaaaaccccctcccatcctctgtcatgcctgcaatgtctttctggctctttctcatctctgtgcattgctgatgtgcttgtcacttaggggcccctcgtgcctgttccttttttttttttttttttttttttttttttgagaaggagtctcgctctgtcatcaggctggagtgcattggtgcgatctcggctcactgcaacctctacctcccaggttcaagcgattctcctgcctcagcctcctgagtagctgagactacaggcgcccgccaccatgcccagctaattttttgtattttagtagagacggggtttcaccacgttggccaggatgatctcgatcttctgacctcgtgatctgcctgccttttcctgtttttggtccatactgtggggacaagatgactcgtggtgaaaaagaaaagcctaggaacatagataagccaagccccatacctggtttcttttcctttttgtgctacaactgcctaaatgcgcatctcccaacatgacctcctcacctctcccttcttcctccccagctacagatctccttatcttcacctgatgcatagacacccaacacaacatcttgttttgagactacacgctattgtggtctgttagagataagactctggaaacatatttcagctacactacctaccagtggtacgcgacaggacacgtttatggaaaatggggatgagaatgcagttttcctgggtttgagagaattattgagtatttatgacttagtaaagaattatttggaaggaacagaccatctgctcaaagggaacattgattgcaagtgtcattcggcctttctctgcctgtctctcactctccgtttgagccacatgatttctgtctgtgagcctgcctctgttttctcaccttgaacactggctttattctgttttgttttggtttgtttctctccttgcatattacccaagtatggctgattgtgactgggtgtaatgacctcccaatcaaaagccctaacgagactccccagaaaacacccaaaggtgaaggtgcactgggaaaaaatgaagttgcgtgggggcatcagaaacaggaggcgtggccgggagcagcaggtcaggagagtgcaaatggaagganangagcaggtnctgctgaagccaagtattggatttccctgcagagcaaacccaaagtcgtttgaaaccgccctttttaatcctctctgaaagccttgccccatctctgctggaaccactactcaaccaccctccttagcttcatggaatgggaggagagaggacacaactctttggccatactaagtggtgagacttgactgcagtacttacaactcagggatcatttttcctgtcaaacttctraatcttataggacttgagaagtttattaagagctaaaaaaataggctattacagtctcacctgaagctcaaatcatgctgtataagatagtttctttagagaaacaccacgtagttactataaacttacaaatggacaatttgtaaattggtgctattttacaacataatattaagtcgatttccttgcatttaaaaaatacatattctgaaataatttagaaacacagataaaaaatacctatgtgtctgtcagaaataataatagctaatatttatggaccactcactgtgtgttttattaggtattaaattatttaatgctcacagcaccataaaggtaggtactaatacgatctgttttacaaaagaggaggttgaggcacagacagcataaggaatttgatgaaggtcacatagttactgaatggggatttcatctcaggcagtccacgttatacttttaactacttcattttcacccctgtgtattaccctactgtgaaaataacccagtttcttcattcatttattcattccactatcgatggacattgagcttgnnttnnattgttttgctattgcagaccttctaagacctttcttatccgtgcctttgtatgcacacgtgagagaggtgttctaacatatatagctaggacagcaatttctgggttaaaggatatgagcacctttgactctctgagatatggcagatggctctccggatgatagggctattttgtatgccttctagaaacgtgtaagggtgctcgttgtctacatcttcatctgcacttgatgttatcaggcttttacaccagatgacacgaaatggtgtcctgtaatgtaaatttgcatctccctgatctccctgattaatggatgttgaacatttttaggttcaagggccacacattttctttttttttttttttttttttttgagatggagtcttgctctgtcgcccaggctggagtgcggtggcgtgatcttggctcactgaaagctccgcctcctgggttcacgccattctcctgccccagcctcccgagttgctgggtctacaggtgcctgccaccacgcccggctaattttttgtatttttagaagagacggggtttcactgtgttagccaggatggtctcgatctcctgactgtagattgcttgtttatctcatttgcccatttatctgttgatttttctaagtgatgttcaggaattctgagtgtttctaaagggtatattgtttctttctaaataaaaatttaagatattagatataaagacaaatctaaaatattaaatgcttgacacatgtgcctttttcatctctcccatgtttagtcaataaggcaattcttttatgtggtattaaagaaaccgattagaaagttgcttcgttgtgtgcttaagtgtgagtcttagaaaggtgaaaagttataattgactcttttagcaccaaaccaagcaggtatatagctgtttaaatactattttagcataagccatagctgtgggaagggtcactccagtgcttgataaatagaagtctattcaattggaccctcattcctcagcggctgctgtgacaagccgttttcccgctgctgctcaggtaggagttttcatctcttgacactttgacagatgtggctgcacacttttgaaccatcttccaacacacccccgtgctctattaccatgaggaaagtctctgacaagagaatgaaagtacatatgcatgagacgtggtacaatggatgtcagtttccctctggtagcatattccaccgcgtgcctcctgtgaggaacagagggaggtaacaggtgcttgctgaaagtaggtcaaacggtggcgaaattattccttcagtgttttaatgctctttgaaaacatttccattgaaggctaagattgaggggaggtggcctgtgtgatgtgagcatttcaagtgacaaagtgacatcctaggcactcctgaaactgtcttcttcgtgaaccttcccacaaactggcagatcatttcaagaattgatggagcccatgagaatgtaggttccctccagaacgcagctctctccccagcccattcccacaaagaaactaggttttccctgcatttccagaaacctccaaaccaacccattatgcttctccttttgtcctctccaaattcaatctgatccttcttcaattctgtcattacttcagtattttgatattttttgactcattctctccactccatcttcatattcctgccctcaaaatcaggtcagtgcttcctcaacagagtgatctctgattccttgattatatttggtcattttcttttttgtttctctttcttctctattgcttcattcacgataggcatttgccaaaaattttctgaattatacgtgattgtacatatccagctgcgttcacgtgcagaaaacatagcctcacacacagcagacccaaattcttactctccccttaggcaggtgtcaaacgatacttcccccaaatgctcacgcatgcacgcaaacagagacacacagtcaaaaacacagcatgcatgtgtacacacacatacacactcatacaggctcacacatgcatgcagagacacacagtcaaaaacacagcatgcatgtgtacacacacacactcatacaggctcacacatgctcacacgcacacacacctcaaacacacacagtgacacagacacacacatgcacactcgtgcacatacacatgcctacacacttatacatgctcatgcacgctcacacaactcacatgtacatacacacacagtgacacacaatcgcagacacacacatgcacacaggcacacacacatatctacacgcacatgctcatgcacacacacttcacacgtacatacacacagtgacacacaatcacacgcacacacatatgcacacatgcatatggctacacacacgtgcatgctcacacacacctcacaatgcacactcacaaacggtgacacactgacacacacacacatgcatgcacacagacatgcatacacacgcttatacacacacacacctcacgcatactcagtgacacagtgacacacagacatttacatgcttatgcacacatgcacacacacatgcacaaacacacttgtacataatcatgcatgctcacacatacacacgcacatacctcacacacacacatacactcacacacaggcgcacacacattcattttcaatttctcaggggaatataaaacaactgcttggaggaaaagtttgtaggaaaaagtgaatataaaaatggttatgcagtaattacctggaataaatattaaaaccaggaaaaataaaatagctacatgtagaaaggggtagctaaaatgctacttgaaaaaggcctattgtaattttgaagtcagtggccaaataaactaaacagagaggccaattgtataaatcctcagttctaaatatcccatatatatttatttaaatatatattcaatatgtatctaatatatcctgattatatttcnaaataataattatatttagaatagacaccagatgaccaggcataggataataaaaaataagattaaaatatcagtgttatttttatttatttgtttatctgtcttactggcaattaaacagttttgcttctttgatttttaaacctcactggaagaaatatgcagaacattttaaaattatttgcatgccacattatctcaaactaaattcctgtctcctattatattttcatcctccagtttgaatcagtcctataactcttgaatgtttacaattatattaattaggagacatgtgctaacttatgccaagcatttttaaaatgtccattggagacaaatttccatcaccaaagatttgttgcattgtcacctttggcaaaaatgaaggatgagtttttctgtttgttcgtttggtttggttttgcaagtcttgatctccaggttaaatacacagccttctttaagacttaaacccagggtttgaaagtgctgtgtattttctcagtgttcaacacaatctgcagtagaagggtttggagcagcaatatgatgccttatgcactctaaagctgctgacaggtttagacataatctaaaatgtaagtcactttgtgtatcaggcagttcagtccttcgcatttgttttaatgtttttcctaaatactctaaatatttcaaaatgagtctttccttaaagattctacatagctcaaaattattttatttatgtatgcatttattaaatatcacaagtgaggtcctgcattgcattagaaatgcagtgtattagaaaggacagctagcttcaattttgaattcataatttgtctcttgagtttaaaacaaaagttttgtgttcaactgcccaccgtttaatactaaaaataaaagatgaacataaaagtcaatttgaagtgattctagatgtaggaatttaattagctgtgggaatgtcaatgacatctcaaaaccatcatggcgtcgtgtcacctgtctagcagccctattgtagtccttcctgacttctcactaacttacccatgaagacaaagataaagaaaaactcacaacaatacaggtaactaagaaagcaccttttgtcaaagtctgttgagattattcagtaactatgtggttggcggagtactagtgagaaaaatataaaacatcatgtatattcaaattgtttaatctaatgcgtccatccttttaggaactcagtactgaagtctaggaacatcaggctaaaaaaaataatggttggatttttctactctttctagacaccagggaacatgagagaaaattattgtggaaggactaatattcttatttttattattacctatgtccagccaagtaagcaaggtcaacacttcaccgttgaaaagccaagattttagcattttcattcttaatttttactctagtatttcactgacacttgagtgtatgtcagaantcagagggtcatgaagaannggacagccctggtgactgccacaggctgagacttactgctggaacatgtatagtgaggcctcctttgttgaccatcaccctgggtcttcttgtgcccaaagcagaatggtgtggcttcctggaataggagatcatattacttgaagcaacaagattgtattgcttgaactctagtgtcctaatttgttttactcactttgtattaatacatgccaaagatagggaaggggatgaaagggtcttgggaccaccatctaggttacagcatccctcttttaggagtccttgctaagacacctcaggggaaactgcctttctgaagcctggctcatggtccagttaactatgtcaacagtaccaacattactttcatttaatacaggagatagagaaatgtttcacccccgaggctactttcctcagaaggaggcagaagatgctccatggccaattgagtacatggaggaaattaagttatatcaaaaattattgttgaatctactatgaatccagccttgcaggaactgagtactgaagcttaggaacaccaggctaaaatatatatatatatatatatatatatatatgagctttttctactctttctggacaccaaggaacatgaaagaaaagtgttgcggaataactaatattttttatttttcttacccatgtccagccaagtaagcaaagtgaacacttcattgttgaaaagccaagattttagcatgtttattcttaatttttcttccaagtcaataatgaagtcatgcaggggtattgagttcttaatccataaattataggttagttaattgaaaggtttttgtacatgtcatcatttacttttcctttctcctactaatccataaatgtaggccacaaagtgtgaggaaaccaggagattttaagtagtgttctataagatagtttaaattgtgaatatccattatggcattgtactctaggaaatacatgttgtcctaaattgaagctttcataaagaaaatttcccatgttttaaatcaattcaagacagatgattttattgggcaggatataggaaaagttctgttttaaatatttcttactttaactgatatatttgtatctctgtagtcttagtggcagtatcccaatgcccagaaatggcttttgtgtgctcctagtgcttagtaaggcatcatagctatgtgactgccatggcattatgattgataaggagtctctaaacagcagaaaaggactatatgtaatatatatgacccagactagagtaacaaagctaaacaccttagaaaagcccagaaaatagtctccccttcccacaagggttattatttatgatcaatgtatgagttttcaaataccctacagcagactcagagatgccataagagctgggggagctgggtggtgaacaatcaagttgaagtccaggagtcctgactgttagcattttctgctttccataatgtaaatgctcccaccatggccaatttcaagtcaccaacatgacatcagtaaacatgaaggtggagagagatgtgattgcagctccagcacaccacagctggggcaagctgcactcccactgcagtcagagatggcatatacatacatacatgtcatacatgtctatgtaaacacgttgcaaaattttgtttgcagaaagtggaatttttgatgcctccatccaaagtacctagtttccaacatcactgtagagggaaagagagagggagactttcgagaaggcacaccctttctcaaagtgacacatccaaagtgatgcatcacttccatttctgttctggttgtgagaaccaatcacattgctccacttcagtaatagtaagctgagtgatgtagatgagctatatgccaggaagaggaaaccagcttggtaggtataatagcttttgctaaaccacaaggcatcctaaaacttaatggcctggacagacactaactcttgctcatgaatttgcggatcagttgaacagccctgctagtctgagccaatctcagctgactacagctgagctcacccctgcctctgcggtcagccactcgtcagctggcatctctgattttgggtgtggctcgccattaattggaatgaagagaatggcagggcccatggccacctggccaccatgcaacaggctaggctgcagggttgatgtggtcgctcggggttccaagagcagcaagagtaatttctaatacacaagtattttccaagtctctgcttacttcacctttgctaccatctcactgaccaaaataagtcacatggctgagtctggagttggtgtggaggatactgccaaggatgtgtggatacagggaggtgaattcaattggaggccactgggttttttcacacaggctaagaggaatttgaaaatcactggcttggcatcataatcaatcctgcttagaggtgatttctttcacaccagccattactggtgctttttccttcttgttaaaatctagtttgtgttgtaaagtcaagaggagctggtgattgaaaaaaataacatccacatgtgtttgttgatttgtgatttcacatctaaccttactcttacagagtagagtgaaagctatactcacatggcctagagataaagggtctgtccaggattcccttactcggctctccattccaataaagagagccagccactgatagggcgtggcagctggcagggtgtccactgaaatcataggctcttggtacctttgtggagatggacatgtggattcccttctgtcctgactctctaaggacgcatcaggaacagacagatgggaaaggcagtctctaaggatatccactggttatgttttcagtgttgatataagtgttacctttttcatataaattctttgtattggacagaataattgtgtacattgatagtagaagtaatgaatatccagatgtcttctaaataagggaagcaaaagatgtcccacttgcaaatgggagatttattttaggttttatgcatttggtcttttacattgtgtccttgttcttatgttcatggctcatatgcaagatggtttctgagaatttcatcatgtaattgcattgttctccaaataagtttatttcaaaatccaattaaaacacaagttttagtacatggctgatgcagtattcagtggaggagactttgttttactaagttgagttgttttactcactcaagcagcaatttaaagacagatttcatttctgagctgtactattcattatcctaatttatatttattgcaacctctgagaccactattttgtctctttgcctacagtcttcagaatctttaagttactgtttcataggaaaaattattggagaaatttaatagaaagagcttttatgaagtacttaagcatttataccacaaaaaaagataagatagctttagatcattttgaaagtttataaatgttcacggtccatttcaagattactaatctcttccnnnnnnacctgatttcttatgttacaccttcaggccttccctgtaagcaactgactgcatctctactttcaaatccttagagtgctttccaagactgaaataatgagagttacagagcacacggtgtccactgcaggctagactctacactgaagtctcatttattttcgttttacagccaaaagatgtagatgtcatcatactcatttcctctgtgagaaaactgagtcacagaaaggtgaaatccagcacagagacaagatttaaactcagttcatcctgtcttcaaaattgatattccattaaaccatggcaatcttaaatctttctgtgaaaaacagattgtcttcagctttcagatttagggttcatgtacttataccttctatccaaccacaaatacttggtgaggaagctgctgtcactcttttttaatatatagagcaatgttgactgatgaattttgactgtctgatacaaggcaagagactacctggtttgggctcacaatgtctaagatgtttctatctctgtgggcctagaaatctacttagactttctgagcctgtttctccatatgtaaaaggggatgcaaaagtatctacctcacagagttgttgaggattaaagatgttattacatgagcaagcaacatctttggaatgaatcttggcacaaaccaagcactccagaaaacagtagctcttatttcaataatccatcctgttggtgtccattttaattgcgtgagaagccctccccgtgtttggcttagacttccacagtctcctgggaggtttgctttcatggcaggactcagaatcaatgcgggaaaaagtgacctttcttgttgttctgcttgaggttggactcagtcccattgttccagactcattgtctcattacacttttggacacaaagcaagatgaaagcgttggttgctgttatgagttttatatctgtagttgtcaaagttacagagaccccacgttggcaagtcaccttgtgtggaaccctatggtcaggtaccattttgtagccctgggcacttttatacnaatcatgtttcnatagaaggtgatttgagctggtatacaagacagtttgagctggtgtctgtcaaaatcgttgacaatgctgtgttagtttcctgttgctgctgtaacaaataaccacaaattcagtgacttaaaaccacacacatgtattctcttagaactctgcagttagaagtccaaaatggacttccttaggctaaacacaaggtatcaacaaggctgcattgctttgggagactctggagaagaaatggcttccttgcttttttcacatcctggaggccacctgcatttgcatttcttaactttatgagagtctgtaatgatgtctttataatgcctccaaatggttgcaaaactaacacatgcttttgtttttatgaagccaaacagatttagttgatttttgaccatggagttggggtatggggacaaggttggcagccatttttatgttagtgtagtccaacatattacattccagccagcatctgtggtattctattctagtcattatagacttttagaatcatgaaactgtgaagttgcaaataatcttataggtcacccagtgcagcctgctattaaggagtatcccctacaacagtgcttctcaaaattcaacatgctcacgaatcacctgggaaccgtgttaaagatacagattctgattcagtaggcctgggatggggcctgagatcctgcattcttaataattgcttccaggttgctgctggtccagggaccacactttgagtaacaatgctcattcctgacagataatgctgccttggggattaatctagtatagactctttgtattttgaaaattccagctgtcatatacacagataagtaatttttggatcaaagccaaactaaaaacatatccaaaactcctctgcttttaaaaaatgtaaaccaaaatgacattgcaattaaaggaaactttaaaaaatcaaatgtgtatgtggagtctgaaggaccttatattaaaatatataaataaaaaagaatatgcacacacatatgtgaatccacatatgtatgtactcacatacacatatactagagatgtatgtgttcatctgtctacatagataggtagagggatagatcatgcatccatattaaataatgcaattgtgaagacctcaagtcatataatattttgtggtactgactcagctttaacattttcctttatattaatttttattagaaaataaaaaattgaaaagtataaatttggactttcctgtagatacaaggatgaatctaaatgtaatgtcatcagcctttttacattaaagaaactgatagccagtttcaagcactggctgaagtgaattgagttgaagtgggccatgccactagctctaactttaaagctggagttactgattttaatatttaaatgtaggtactgcacatatttatcaccttaattcttatttaatatttgaaagttactgctttggagaaagtcagctaagcaatggtgattatagtgtatcaaaatccattgtaagtgattattttaataactctgaaccaacagggacatgtagacattcagacaaaaaacaacctaattataatgacagcaccaaagtgaagctgtgacagttacaaaaccaatttaaaagtttcctgacagccatggctaaaagctttgtttggatctttcagtaataacagtttacttaagacataaatccacagctaaggaaaatagaaaaagaaagttgtggcttttctgccatgcagatgaaatggaaatcagcagaaatatttctaatagaacattatgctgaaaggagtatagaaaaggaataatagtaatggaatttaactctcacaatgcaagaaagactaaataacaataaaaattttaatgagcatagaaatgtgccagatgatttgcctatctgtgttatgaaggtcagtcatttctgcaagtaaattgatgtgcatataaacatgttgattggcagctctggaatttgatttttgtattacctgtaaaataaagtcaaagggaaacggatatgcatcaatagccacggtgacaggaagccttaataaaagtctactttcccgatgtcactggcatgtctcaaattattagatccttatcaatgttacttacctcactagactatgggctgagtctgatatgcaacataattaattcttcaacctcgggtaattaatttctcattcccgaacagaaaaatgcttgattcacaaaattgcattccttttttctgtaaacctttgtagaatatttgtaagtgtaatcactgttctaagccctttacatatattgactcatttaaaattaataacaagtctttggagtaggtactattttctccacttnatcaatgagaaaacagaggtttaagtaactcccctggggccacagtgccagtaagtggtggagttacggctccaggcagtttggcccagagcacctgatgtaggataaggaagaccccatgaaacacatacatagagtcatggagacattggcagtgctcagaagaggcagttcttgttctcttaccccattttagtaataacagtttgcttaagacataaatccacagctaaggaaaatagctctttgagctgcaaggactaaaggattctcaggtcagaccagacagtaagggcatgtgttaataaatacccagaagaaatgaaacctcagaaagaggatgtgtggtggcgctagtccctgggcagatgtggctcagtggtgtgttgtttgtgctcagttcctttccataggtctgcttctctctcctctttctgctgcccagggccttcagcctccactctctccatttaagtattctctaccccacagtgggcagcgggccccagaatggactttaaatagactagggtaaaatgacttagtctctctgaactgtgtgaaatgtgagtaatatatctcaccatatagagctatggtggatattaaagtagataaaacaaataacatttgaataatgtggattatgaccaagtcggatttatccctgggatacaaggatgattcaacatatgcaaataaatgaatgtgatacatcctatcaacagaatgaaggataaaagctatatgatcatttcaattcatgctaaaaaagcatttggcaaaattcaacatctcttcatggtaaaacccctcagcaaaactggggatagaaggaacatacatcaacataataaaagctatatatgacagacccacagctagtatcatactgaatggggaaaatctgaaaagcctttcctctaatacctgcaacatgacaaggatgcccactatcacctctgttattcaacatagtactggaagtcccagctagaaaaataggacaagagaaagattaaagggcatccaaattggaacggaagaagtcaaattatccttgtttgcagatgatatgatcttatatttggaaaaacctaaagactccacaaggaaactataagaactgatgaacaaacctagtaatgttgcaggatacaaaatcaacatacaaaaattagtagcatttctataagccaacagcaaacagtgtgaaaaagaaatcaagaaagtaatcccatttacggtagctacaaataaaataaaattccttgcaattaaccaaaaaattgaaggatctccataatgaacactatcaaacagtgatgaaagaaatcgaagaggacaccaaaaatggaaaaatattccatattcatggattggaagaatcaatattgttaaaatgttcatactacccaaagcagtctacagatgcaattgaatccttatcaaaataccaatgatattcttcacagaaatagaaaaaaattctaaaattatatgaaaccacagaagagccaaatctatcctaaacaaattgaacaaagctgggggaatcacattacctgacttcaatttataattcaaagctacagtaatcaaaacgccgtggtactgccataaaaacagacacatagaccaacgaaacagaatagagaacccagaaggaaatccacacacctacagtgaactcatttttaacaaaggtgccaagaacatacactggggaaaagagagttccttcaataaatggtcctggacatccatatgcagaagaatgaaactaacccctatctctgtaaggtacaaaaatcacatcaaaatggattaaagacttaaagctatgacctcaaactatgaaattactacaagaaaacactggggaaaatctccaggacattggactgggcaaagattttatgagcaatacccccataagcaccggcaaccaaagcaaaaatggacaaatgggattacatcaagttacaaagcttctgcacagcaaaagatacaattaacaaagtgaagagacaacacacagaattagagaaaatatttgcaaactacccctctgacaaaggattaataaccagaatatataaggacctgaaacaactctataggaaaaatctaataatctgatcaaaaaatgggcaaaagatttgaatagacatttctcaaaagaagacatacaaatgacacaaacaggcatatgaaaaggtgttcaacatcattgatcatcagagaaatgcaaatgaaaactacaatgagacatcatatcaccccagttaaaatggtttatattcaaaagtcaggcaataataaatgctggtgaggatgcgaagaaaagggaacccttctacactgttggtgggaaggtaaattagtacaatcactatggagaacagtttgaacattcctcaaaaaactaaaaattgagctgctgtaatttagcaatcccactgctgggcatatacccaaaagaaaggaaataagtatatctaagagaaatcttcactcccatgtttgctgcagcactgtttgcaataagatttggaagcaacctaagtgtccatcaacagacgaatagataaagaaaatacagtacatatgcgtaatggagcactattcaaccatagtaaagaacaagatccagtcatttgtaacaacatggatggaactggagatcattatgttaagcgaaataagccaggcacagaaagacaaacatcacatgttctcacttatttgtgggatcacaaaatcaagataatcaaactcatggacatagagagtagagattggttgccagaggctgagatgggtactggggggacggggggaggcagggatggttaatgggaacaaaaaaaantagaaagaatagataagacctactatttgctagcataatacagtgactgcagtcaataataataactgtacattttaaaataacttaaagagtgtaacataatcatttgtaactcaaaggataccccattctccatgatatgcttattccccattgcatgcctgtatcaaaacatcttgtgtactccataaatatatatnngtgtatatatatatanntttatgtgtgtgtgtatatatatatatatatatanncacttactatgtacccacaaatatttttaatgtgaataatatggcctggaacatagtaaccgtttaatacatgataacctttataataatactcattatggccaggcatggtggctcatgcctgtaatcccaacactttgggaggccgaggcgggcggatcatgaggtcagaagatcgagaccatcctggctaacgtggtgaaaccctgtctctactaaaaatacaaaaaaattagccagacgtggtggcgggcgcctgtagtcccagctactcgggaggctgaagccggagaatgacgtgaatccaggaagcagagcttgcagtgagccaagatcacgccactgcactctagcctgggcgagagagcgagactctttctcaaaataataataataataataaataatactcattattttttaatgtttaattatctttatcttaatgtaggcagtagacttggaccagacttgggttccagtattgagtccatcttcaatccctgtttgacagtgggaaagttacttaacctctttgagcctcagtttccttattagaataggttcctagggctgttatgagttaatttatgtaaagcacctagaaaaactccaggtagagagcaagtatgatgtacatgtcacttactagaagttgtagtaaaattttcaacaaatatttatggnaagtaataagtaaaaaataaatctcaggtttctcttacttttccagttgtataataatcatctctaaaatattttcatctgcagtattctccctaaacacagaataaagaaatacttgttttgtgttactagctttttaaaagccatttatccatacagtcagaattaaggataaatactacccaaccaaccaattaaaaattaagatgcaagccatacacaaatgatatatgttttccatttttagtcaaaagtctctgaaaccatcttcctagtcactgtttagctgcctgaaagaaaggttcccttgcctgcccagttcagcagtgtgggacatggatggctttatcttggaagccctgttttgcatagcagcacatcacaaaggtgctgtcagccaaggactaagtcaattacacttaatcatgaggtttatttctttgttaagagtctgtctgtttcgccccctcagaaaatgatgcggtcatccgttttccacatgttcatcctgagcatggtgaccgtggacgtgatcgtggcggctagcaactactacaaaggagaaaacttcaggaggcagtacgacgagttctacctggcggaggtaagcagctctccaccgtgcagatcgctgggcaaggagcaggtgaaccgcggacagtgcctcttagagatgcaaaagtgaagtgtagcctgttcattacttgactttcaccaaagctgaatcattgtctgcttcttaagtggttcctctggtcacagtgtggcctgattcatttcctgtcaccgcgggcctgagtcatcttcctcacagtatgtcacgttgccagggtaacccttttcccctgggtcttactctagaatcttgctgttttcctatccacctcttattatctctccctttctcagcgtacattagtttgtttcctactaagtacaagcaggcacagattcagaagatcataaataagctggtcagctcagcaatacgtagaagagggctggaaggacatatagagagatggtaatgataaattactcctctagaaacgggtgttacaacctacattaaagacaccaattaaaggtaggtttcatgattaaataagcatctccctagcactctacatatcatattacagttttggaatagtacttcctattgataaggtaattttgttaatacctgattcttaaaatattttaatttttatttctttcaataagcattttgcatttatattaaagcattttaataattttccaagatctttgtttttttaatgtaaaaacttacctaaaatatatctcagaattcaatggttacatatttacaaaaaaattgattgacaacgtagaaacccattcattgtgtgagtgaggtatacatacacagttgtacaacgcttcaataaatccaacctatgaaaaagtaaactgaaaagacagaaaaaagtcacaattttattatttttaccaaagcatttttcacataaaaaaaatcaatttgccatataattaagtgttattacattcctagaattctctcagtacatttaaaaattgatttggttaataaaaatttagtctacccacgcacaagcttttctgaaaaagaattgattgcatgtacctaacgagcatagacatctacgtggatatcaagaaacttttaagtagtcgcttctgaattctcctcttcatggaagtatgtctcctgaaacaaaataccgttaatacaaaacttttactgggctacagtctctgtgatgcaacatttgcccaatggccacctggtatatggagcttacaatctagcaaaaaaaaaaaaaaaaaanncaataaacaataagagttttgtgctatggagaactaatgacaggggctgatgaagaaattatatccagcccagcctctgaaaaataggtataattttgctgaaaatccagcacacgatgaatggattgtcaataaaagcagagagaaggtcgggtatggtggcttacgcccgtaatcccagaactttgggaggccaaggcgggtggatcacgaggtcaggagttcgagacaagcctggccaacatggtgaaaccccatctatactaaaaatacaaaaaattagctgggtgtagtgtcgggcacctgtaatcccagctactctggaggctgaggcaggagaatcacttgaacctgggaggcggaggttgcagtgagccaagagcgcaccactgaattccagccagggtgacagagtgagactccatctcnaaaaaaaaaaaaaaaagcagaaagaagcctagactgatactaatatttgcaggtaaatttctaacacagggaaagaacaatgaaggaagcctcatcaagttatttctgtttttcccaggaagtaggaagcaaggtctgcatccagccagatgatgagacagtgaagtgggaagttggcaaagaaaggggagagggaagaggtgcctggggttaggcaaaaaattaacaatacagcaaatgcaacattaaagatccatttaggactatggatttacagtgataccaattcagatagctctgcgactttttagccctgtataagaagagggaaaagcaacatttaaatcaatccacctttagggatttttttcaggttaggggaactgagaaacaaggggaaaagatggattgctggcaaaggagttgaaatgatgaccctgtggtttctgttggatgtggaaatgggaggagaatggaggagtgaaggtgaaggatcaatggtctctatatggttaaaggacgggtaccaccaaagtgatggaataatagaaccagaagaggttatgatcagagagtggagtccaggattttacaattccagggtaattctaagtaatgaaatataaaatctagggtgtcacgtgaatgtgggtggcttaaacatattgatggagtcaaggcagaaatggaaacgactgttgaagctaaggcgctggctgagtcttcacatggacattgaagtcactcagagtgaggccagttcttggagagaagagtggagtcagggagcaaagtcacggggaatgagcgagtgactgatgaacagattgacagaaggacagaagacagctacagggagagtgggtgacagcatggtttaacctcatggaatacgttgcaaaggagtatcaaggagtaatggatgggcacctcttcctcgtctacatgttctactagaaagggctggaaagggctgcaggggtaactcatgtatttgagagagaatcagttgaggcacagacagaaagtagattctgggaacagagattgataatgtagaggacattatttaacgtaggagagactcaaagattagttcaaggctctgaggagggagaagtgggaagatcgatgagacagaagaatgatacaagagtaatagcatcagtaggtaaagatttgctggcaaaaatgccaggaaacaaaagcagaaatgaccacaagagattacagttatgyttatctctctgggaggaggaatctgcatctgctcctgagggaatcttgacattagagattacagtaacattgcttttgtgatctagcacagggtgggcaggtggtcacactaaatcagtcctggagcataagtgaataggccctccacactggcaacagcagggaagggaattctttagtaaattttacctaactttttcatgtacagaattgatcctttcctaatgtgtttatgtatggtagatgatttatcctcataccttaagaaattccaacaaaatgtcaaataattgaatgttgtagagttgactggttactatttaagtgtattgtagagtccaggggtagttggtaaatccttgtcttttttcttttttatataatattaaaaataaaaacaacctcaacaacagacagacaggattggatggtggaattcagtatatattatgtgtccaataattgttgtcccctgacaataataataagaaagggtgtgggaggaaactttgagaggtggtggatatgtcaatggcattgatggtaatggtttcacaggggtacacttattcccaagttcatcacattggtacattacatgaatgcagcttttaacatgtcattcattctttttttttttttttttttttttgagacagagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggccacatgtcattcattcttaacaaagcggcaaatcagttaaccagctaaaaactaacttgaaatcctggaaaaataaataaataaataaactaggtagtggtgattttgttgacttaaacaaccagatagaccccatcagtgtactcttgaactacttaagcattctgaatttgttccactctaggttttggggtttttttgttgttgtttttgtctccttttttttttttttttttttttgagacagtcccactctgttgctcaggctagagtgcagtggcaccatctcggctccctgcaacctctgcctcccaagttccagtggttctcttgccttagcatcccaagaagctgggataacaggcacacccagctaatttttgtatttctagtagagatggggtttcgccatgttagccaggctggtctcaaactcctgggctcgagcgatccatccatcttgacctcccagagtgctggaattacaggcgtgagccatcactcctggccgttccactctgtttttgattttgtgtgtttgactttccccaagtatgtttcgtttttattcagattacattgccatttctctttgtatcagaaacttatacatgagacaaagtgatttgtttcaaatgaatgtgaaaaagtcactttaattaaacttactttttcataatatattctatttttataaagtttataaagatgtcttgagtttaggccagggtttggcaaactctttcttgcagacccagatagtaaacattttgccttttgtagaccaccctctctctgtctcaactactcaactctgaaattgtacaaaagtgagtgtgtctcagaatggttgttttccaataaaactttatttacaaaaatggatcttggtttaagctatagaaacagagagcaatgtcaaataattgaatattggattatattcaattatttaaaatacaaataaataataacattttgtgttaaagtcaaatattttaatactgacaacaataaagctatgatttttggggggcccattatgttctaagtgctttaaaagaattcattgtctcatttaattctcaaaatagttataataacagcaattcaagtttatagactataatgatgcagtatgtaaggcaatagcattatgtaatattttgaaaagtttattttaaaaatggatcacaacaggtcatgagcttttaaagaagttattttttaagttcccacatattccagaataaattatttagaaaatctaaaataaagcactttacccattaagagccaactcacagtctaaaaatcaccctttaagaataaattaagatacttagcttctgttgtgaagctggggacttgttttgttactttggattaattaacctacatttagaattagttaagaaacaggaaacagtttacctcttttgtagcctatgcataaacagaaaaaggtaaagtttattttcctctgtaagaattttaacttttcattgtgatctaagagaatatattattattgcacattaaatcatatgttataatgaacatgattacattttaaaatatatattattttcttaagaaataaaatataagcaatatttttcttattattatatgaatgtatttgtatccattagactgtacatttaacaaaatttatcagcacagcacaggagattaaaagctttatcttcttttatctcaattcttcttttgagaaactcacaaacagatgggagagatacaacaaggaaggcaacggacctacagagaggcccaaagttagctctgtgtcaccccctagcattatccccagctatgttctctgatttcctctagacctatctgaatggggagctcaatcctcttccacctcctggtgttaagtattaatgaggacagtcatgttttccaatgaagccagaggggagaaagaagtcctaggactctgtgctacataccccacagtaaattaactagaactcctcttaccctcaactctgtctatccctcctacccagcctactcccatatctccccaacttgtcatcagccaggcaggctcttcccctgctactcatgcacagcaattccactccacccccagacttgcatcttcacttgctttttgttcagcaaaacaaaaagctctgctattaagaggatctagattcctcagaaaagaaagtatcatctattacttaagccctcttagtcccccacaggaaatatttattttaaatacattcagagtttctctgcaaagatttcacatggatataaaatttttcatctcatttcaattataaagtcagagaacagagaggaagaagggcctagtatcctttcttacatggcagttgtgcttggtgatggggaatcagaggtaaataaagtaaataagatatggccatcgatgtgtgactctaaggacctctttctactcttagaagctatagttctgtgagattatctggtgtaatcttgagtgaggtgtatacctaaaatttgtggcaataacatgatttatcaaagaaattttagtgactctacaagaagaaattaagaacaatttccaagtcagtgtttgtctcaacacatcatatccatatgagcagttttagttattaaatccgtgtttccaatagatctaaaaatcattctctattgtggtagactaaatatgtattaagttgtcttgtcggagggaatgagtcataaactgccaaggtgctagttcgctgagagaagttacagcttgagagaatgttgtttctaaaacagattattgatgaagcaaaacgtgctgagactaaattaattttacacaaataagatttgggcaggattagacttatttatgtcagatacctatgatgtcagaagcaaccattagcctttgtgtattaatatgattattttgcaggattctttttctatggttatttttccttctttttgaaatttctatcctttgaaaatctgtgactttcttttatttccttcttaattagaaagtatttcataaattcaacacatagagaaaatattataaccaatatttgtgtgtccgtcacccagatttttaaaaaattaatagtttttcatgtttgggtcaggtttttcaatcagaaataaaacatcattagtagagtagaagatccctgtgtaccacttcttgatctcctccttctccctaatccacagagaactatattcatcatatcaatgcatgctttataatagtatgaagtattaaggtgccactttttcaatcctcattatatttgggaaattgattcatgtcgattgaagcatgaatttatttgttctgcatgtaagggcaatgggtatttcaattgcttccaactttttgctgttgaaaacaatacttcagtaaacattgtcattagaatttctgtgccacatttgggagagctctttctaaatatatacctagacttaaaattgtgaatttgtcagatatgctcactttcaactttgctaaatattgttaaattgaacttcaaagtgcttgtgtcaatttacattcctaccaggatatatgagcagctctacttcatactacttttttttgttcaggatttttaagtttcttacaatctcatataatcattatagtcttaatatacattttcttggttaccatagagatttgacctttttttcgtattctgtcagccctgaatttcctcttctataattttgcctgtttaatctgtaccctaattgatttttcagttatctagtagtatgcagtgcagacatctcccagccttcagcttgtcttttaacttttatttgctgtcttttcttcaatagaagttattaattttaacactgtaagatatagtcatctttaatttttgattcagtgctttttgtgtcttatctataccagagtcattaaagacattctcttatagtaaaggctagagttttcaagttgtgctttttcaattttagactccaatacatttggaatgagataaggacttgattttttccccatatataaacaattgtccttcgaagattcattgaaaattcattctttcctcactcctgtagtgcaatttctgtcatatatcaggtttacacatgtgcacaattgtgtttctggacacctcagtctgtgtctatacatcaaagctttagttgataacatgttgtcataagtgctatcctttataataaaccttgagatctattagggcaagctactgatgttgatattcttcttcagatttgttttatctgttcttggtcttgctattcttgctctacattatgaaaatttaattcattttagtaatgagattttaattgatgttaagaacatctctattcagttattctaaccttccttagtgttttccaacaatgctttataattttctccaaaggtcttccacattttttgttacattcatgcctacatatcctacagtttcttttgtgaatgaaataatttcaattacatttttattgatgtaggaataatattaattttttatattgacctaattcatagccagcaactttactacaattggttgtttgtgatcatccatagacttgattggaatttctatgtagataattgtatcttctgaaattaaggttttgtctttccacgtctaattatcatatctctgatttttttcttcttttattatccgggtgtgggtctctaataaaatgttgaatagaggcagtgatagtggacatgctgggtgtttttctatattttgatacgaatgcttatgatttcatcattaatgtcatttgtaggtttttatagatgcctttcatcatgttaataaagctcacttctatttctaggttgccaagagcatttctcattatgagttgagtattcaatcttcttctacctatactgagctgactacacagactttcctttttaatttcttagtgcagtatattagaataatagatattttgatactgaaactattttagattcctgaaacacttgattttatagcattacttttaataaacaaatccaataagctgacattatattaggaattttcaatcataaggatagtagttctgtgattttattttcttttacattgctagtctgattttgttagggaagttacacttcctttcacaaatggaatagacatatttttctctttttctcttctgtgaaagaatgaataaagtccctctggaacaccatctagggctattgtttgcagtgggatccctggtggagaaagggagaagagagatcttgattaatgacttattttatggtttgattattatggtttgtcagtttcatggtttattgcttgctatttatagagtcattaattttaactaatgtctcacatttccacagttttctctcaggatttttataatctctattgaaactgaatgtataatcctttttgctgccaatattgtttatttataactttcttttttatagaagtaaatgacttctgatgctaactacctggattttagaccaaacttcacagcacagaggcacagtcctctacaagattacccttactcgagatactaactgtaagctcagggggttccagggcctccctgacatatgacatatcatatggctaatatggttgatatgacatatgacatatcatatgacatatcaacaggctaaaaactcgggggtttccactagccttctcaagtttgataatttgctagaatgactcacagaaatcaggaaagtgctatacttctgattacatttttattaaagcaaaaaggttgcaaatcagagccagccaaaagaagagatgcagagagaaaggtctggagagtcctacatgtgaaacttgctgtttcctcttttcatggagtgaggacatatcaccctctcagcacatggatgtgaaacaatattcagagcgctgagaaccagggaagctcacctgagctttgtgctgaggcttctgattgggatttccttaggtaggcatgatcgctggaatcactggccatgcacacaaggttggagtttctgacatggccagtgctcatgaatgatctcattgccataaactaccagggtcaccataaataacaaagatacctatcaattggcaaattccaaggatgtagaggctacttcccaggaacttgaggcaaaagccagtaaaattctttatttttcattcaaagaaccagcttttagtttggttgacactaacagtttctaatgtacctgaaattattggttcgttcccaatattttgaactattaagtccctttatttctttgttttttaaatttctcctctcacctttattattttctctttttcatttatctctttggtcatcttaaacttctttgaaaattcttactggggttctctgggtggaatataaatcattctattataaagatacatgcatgcatatgttcatcgcagcactgttcacaatagcaaagacatggaatcaacccaaatgtccatcagggatagactggataaagaaaatgtggtacgtatacactatggaatactaggcagcgataaaaaggaacgagatcacatcctttgcagggacatggatagatcgggaagccatcatcctcagcaaactaacacaggaacagaaaaccgaacactgcatgttctcacttataagtgggagctggataatgagaacactgaatagctgaacatggacacagggagaacaacaacacacactggggcctatcaggaggtggggggaggggtagcatcaggataaatagctaatgcatgtggggcttaatacctaggtcatgggtttataggtgcagcaaaccgccatggcacacatttacttatgtaacaaacctgcatatcctgcacatatatcctggaactttaaatttaattgaatttaatttaaaaaaataaaataatgaaaacaactaaatgatggtgaaaaaaaagtttgacaaaattaattttactgagagtaaattcatagtttgaattttagaattttgttttctttctcattagatttatttaaaatctttgcatgctgcctcatttggcttgggagattttcttatttctctctccctccctctatgatcacttctactttcttagggatattgcagttgccttaaccttgaccctggaccctaccatccagagccaggtttttaagtgtcatttggagaatgctagccccttgatatgggtgatatcacagttcttgtcactagtctatattccataaaatatcttgttctgatttttcagataagctgataatttctcctcctgcctctggaggcctgcagctgcctataaggtcttagccttgggtagttggtctctgtcctttccttagcagagaaacccattctagtccctgtcttttcactatgaaggttctcatctttaccttatctaaggtgccttagtctcctcaccctataggaagcaattgagtccaacattgcctgattcttgacctagcatatgtttctgttgcatttctggtcaacaaagaggttaacctcgtttagaattccttcctgcctctttggtttcctgttttttcctttgatcacagtatgtttgaaacagaaggggtttcataaatcatgaactcattttgccgtcttgcagggagtcctcttttctagaaaagtgatttctgcattcctctgaagacatttggttttatgatatttgttaaaatgaagagtcatttattttggccccaacaaaatctgacacccacgtttgagttcattaatcataaaaattggccaaatgaagaaaataatcaatcttgccatagcagcagtctttgacattattggtgagtttaatttcattttctgggaaacttcaagtaaataggtttcttctgacanntactttttggggggtgtttctaaaagatctggacattatcttgttaggtaaaaatataatacaataatgacaatctggacttctcaaccatccatctcaaaatttgctctttataacatactgatatacactataagctaagagtaggtgatttacagacagctccagactgatacaggtaatcagtaatcactttttgttgttattgaatctgaatttcatgtaagagaatgggattgcctgaactgattttcttgtgagctttgtcctgtaatcttcctttgtgataaatatgcatttttttctcatgatatccatcatgattctttgaaattagaaaacagagactgcaacacaaatattagataacacatttcaaatatctttccactcatgggcaatgtccatctctaccattttatctcataggcctgtgtttacgagattagttaaactctgctgttgttactggtagagggtctcgactgcaagttgtccaggttcttgacgttttgaataaagaactggacaaaacgcacagcaaagcaaggaaagaaagaagcaaggaaagcagagatttattgaaagtgaaagtacacttcacagtgtgggagtgagcccaagcagcagctcaagggccccagatacagaatcttctcaggtccaaataccccctagaggtttcccattggccacctacttggtgttcaccccatgtaaatgaagtggttgcccgcaatcagaggctgaggtgaagttacgaagttacattcctaggcaagtgtctggttgggaaaagcaaccgatcagaggtactttcaattatccatctgccgcacagaaaaggtgggggtttgcaaagggagtagcccctggtccttttgctactcaggcatggaaagttagggtttttctttcaatttagttctaagaagtcagtgtgaaatggacttaggttccctgcctccagaccctattctcctgcctcattatggcgacatcaaaccaggtatatcggttgagcatgcattctcaacagggagtaaaatcacctccaaaggggtgaaaatatgttcttagttggggggtagaaaattctctcttttataatgcagattgccttgtagaccaaataaccaaagtagagtatgtaaaaagatatataatatggtagaggtatcaaaactccatgaggtggtgattaagaacaaaatctctaaaagacttcttagaagaggtgatttaaaaaaagattgggaaacactgctttggggtatttgtaagaagaaataaatatttctcttcttttctatgcattgtttctaagttttcttaaagtaatgcaaaaaaaaagaaaagtttattatactcttacaaattttgtcttaactcttaaaaagttaagttttaacttgtaggtactcacaaaggtggaatgcactgcagaatatgaaacagaagaagggtcccaatgagcccttcttggtgcaggggctgcagactgaggagggccaaagcagtggagggctgccaggctaagagtgacaaatggaagccaccgttccacttttccacaggctgcctgctgaatgcctgggatttgcagaaagccttgagattggacacctgcacaggtgactttggtgggagccctccaggcaactaggttctgcagagaccccagttgccttctcccttttagatggctcccagcatgcccctggcaaattccaccctgtccagctaccatcaaggttgagttcactgcaataatctggagctgcacattgagaagtggttactagagcttgcctccactgcttctattttggttcttctgaactattatagtcctctgaatttaagtcctgtaccaaaccccataacctacactgaaatacttagtgatcatttaaactaatttgattttcctaatcaccttctttaaatcctgacacagttcttctctatggcaggataacccataccttcaagttaagttaggcatatttttatgtgcattgattttccataaaatgttatcactttggcatgaatatttcagagagctagagtgttggcttgtaaaagttaacaaattatgttcaaagcaagccatttgattccaagagaatctggaatcaggaattctaggggaaaatcagtgagactatagatatttaaatataatcacataacaatttgctaaatcttttagtgccttttagtgtctttcaaaagaatatcattttcccacagagaaaattgtaatgcaaacagatccagctgcatattttactataaattatgctcatttaaacagttcattccatctgtccttttgataatatatgcagttagtaaggtgattaattgtatgtatacataattagacaatctattggaatatctttcaggcattcattagtagttttcttttaaaaaaacactttaaaatggatcaccactctttttttttggaatgaaaatctcagtctgaaaatacaggtgaagggagagatgatgtaatgaatgcctattactgcttcctctgctaatacagcaaccaggtcaggcatgtggatgggaccttcatgacacatcaatttcagtttctcctgagattccctagtatccataggagagaaaacaggttcaagtcttccccatatcattgcaaggccagaactaagaagcctgtgctagagaggaagtacctgtcgcaggacttgcagcaaagtctgagtcctctggtcccctgaggccactgctcagggcagagagcctttcatttcccgcagaggagctggacctgcctgtatgctgcttgtggtccaggagcagtccacagagtcctgtgggtctcagtgggccaggaagggaagctcactagagactgaattactccaggtggagaagggggtcaataaatgactccactcatttctgggaaatcctgtcaccaaaaagccttcttcagatgttcagcaaaggagctatggcttttcaggtctttcgcctccgtaaactatggtgccctctactacccctcatttgccactgcttaagatagcataaaagacaaacgacaattacatcaaaaacatgtatccatggtgaaggaaagaaaagttaactcacacacaaagggagactcagaaactgaggatgaaactcagagaatcaagacaaatgtctagtctagagatttcccaagcaagtagaattgatgggcgagagaatccgttgttgttgttgttgttgttgtctcaagaaaaagtaagcagggcacaaaatacaatttctgtaacttcaactaaaaatgtccaaaaaatgagttaaaaaagggcatagtccttgagagctagtttttaatatgggagatgaagcaggaaaaaataactcaaaaagtggtagagatataataataagggcaaagtaaaatagcctatccacacagctatgaataataggagtccctgaaggaaaaagcgaacagatagaagaaaagttgtcataaacaacggaagaaagcttacataaatgaaaaaaaaaaaaaaacataaatctgcaaactgaaagagctcactaaattttagtgagattaaatgaaaatagatgcttactcatgtcctggtgaccttcctaggttgcaagcataaagaagtaaccttgttcagcccctaaacaggctgaacctaaacctgaaaaagtaaaataaatgtattgtgcaatggatgactcatctacaatactggaaagtggaaggtaatgaatcaatatacatagactactgagagaatagaactgcaactacagaatctgaacacagacaacatgttcatctgtcagtgttaaaaatgcattttatatacaaaaggactaagagagcataaaaccctttgtatttatttgagaaaactactttaaagtgtgctaatcacaagataagtaaattgcaatctcaatgactagacaaaagaagagagaattgatgggcattggatctcaatagataataataaatatgtcaaatagggatctttgaaaaaagagaaatgataacattaaagaaactagtaataaatttgtaaatgatttcagcaaaaccaggacttaggaaagggtgtgatatcaaaggagagtgggatggtagaagtgtactatgaagtttgcagatgtgtggggtggagggatgagtgagccagcaatttagttgtgaagcaataagaaaaaagcattggtttgatgtctaaccattgagaattaagccaggagattagaaaaaccaagtcgagcgtccaaattaatgaatagggaaagaaattagcaaatagttgaccaagttaaaaaaagacaagataaggttaaaaatgaaacagcaatgacataaaatacacaggaagcgtaagaccaagtataactttggcacattttgtttgagcaggttgaattttcttatcaaaaaccctagaaaatctgagtatgtttaaacacaaaataaaaattgtacactgcttataggaagcacatacaaagtggccctgcaaggtaactaaaaatcatttctaccaaacacttaaaatattaagacgttttaaatgttcagcagagtgtgcaaaaaattaaggaaaattcttgaggccaaaaattaaatgtgacgtgagtccagaataccaggtgggccccaaggcagtggctgccctgaggacatttgcaaatccttgtgaccagaacttgcagttttcaggagtgtgtccaggatggaagtgactagaggcctgtgcaaagtgaagaggcagaagagaggccccacataacatcaggaaggaaaaatcccatccctcagagggaaaggatgggatcatcattctggaccttgacccctggcagatcaaaaatgttatctctggccgggcatggtggctcacgcctgtaatcccagcactttgggaggccgaggtgggtggatcacttgaggtcaggagttcgagaccaacctggccaacatggtgaaaccccatctctactaaacatacaaaaattagccaggcatggtggcatgtgcctgtaattccagctactcgggaggctgaggcaggagaatcacttgaacctgggaggcagaggtcccagtgagctaagactgatcatgccactgcactccagactgggtgacagagtaagactctgtctcagaaaaagaaaaaaaaaggttatcatgttatctgtactgaattgtgtaagacagcctgcccactcgtggggttggagctcaaaatcccattacgtccagaatctgagaaacagcagccaaaagttgatttaagggggacccaaatttctaagatgacccaggttccctggaagaaccaattctttctggaaaaatatgtaccctaaatgcaggtcttgaaaatctagagacaatcaatgatttcctggcaaaatacaaactgccaaattgatggatgagataagaaacctgatctgaacagcactggacagctttgctccaccatcaccaaaaggcctgtgcccatgtgggtttcctggtgctttctgtctacttttaaaggtgtgctaattcttattttatttcagaaattttcatacaaaagataaaaactgtacagaatgaatgataaagctagcataatcttagccaaaacctgataatgatatacaagtaaaactataggccaatttcacataaaacagtaactgtgaaaattataaataaaatatattacccaataaaatccaacatgttagcattatcatctagagtttttttccaaaaataaataaatggttaacatcaggagcttttgtgttatatttcattgcctcagtgaattaaaggttaaatcctttgaaggagaataggaagaaaaggaaagtctttaaacatgataaaaattaatcaccaagactcatttcaaccactatatatgaacgctgaagaaactttcattgaaaacaaggagcaagttatggattcctattattaccattttgtatttagcaaccattatataactgtacctttataactaatgcattcatatgattaactccaaaataaatcttaaaaatgaaataatcttattttatttccttatgaaattgtgcacatttctataaaaatcgtgagactttatgaaaaatattagaattaatagaagaatctagtaaagtgggtacagaaggtcaatatagagaaatcaatagcttttcttcccactggaactaactgggtagaaatagtaaaaaagaaaaaatacatagatagatagagagagagagagagaaagaaagataggatatttaggatatttatntnnannaaacttattaaagaacgtaaaatccagttcaaataaataggaaaagagtgcattcctggatatgaagactagtattaatcttcttaaagttctattttatatgtaaaaggtaaagnaaaaaaatcataaagttgtagacatatgtgaacatgtaaattttatgtttgtagcactaacaatatcaaagttagaaaccagactgttggctagggaaaaaatgtttgcagtatatatagcaaagattattgtcctcaataccaacaactcccaaaacgcataaggaacacaacataacaccatataatagtgtcaaggttacggacagtaacatttaattactttaaaaagaaattaaagtgattaataaacatataaacagatgagcaacaataagtttgattacactgatactggcatggatttgagacaagataactttcctgtattgctgaagggcaaatgatttcttataatattttgaaagtaatcactttaatattttctaaatttaaaacacactaatctgtctttgccccagcaataccaactttggaaatctatttatagatatgaaagcctcaataaatactggtattgagggaggatgcttagtgcagcctttttattgaagtagcaatgtgcttaaccaccttcctgatgattcctgcaaataccacccatcaatgaattagtagagaaaatgttgagttacataaacattgaacactatgggaaattatgcagctgataaataaaacataggacctatatcccttaatctggagggatatccacgattgttagtgactcagaaaacaagttgcagagtgatatataatattcctgcatacccaaatctttatcattcacagttcacgtaactttaggtaataccaagaatgcctatttgtctttagaattttttttctttccagagtaaatacctgtcacattatttttaacaacttcatagaactgtatgtaatatttatacataataagtgtatagaaagtatgaacttgacagaggacaatgacatctttaaaattagaaatagtgctttcataaattgtaacaattgtttttacatctcatgtaaaaacatgtaattgcaagcctgttnctgtgattgcatatgtgtggatgaagaagcagatgatcaaggaaagaatatacatgacattaacatgaaatacctgagaaacntgggcaaaggagttcatnccacgtgtttngtggacttaagatcatttcaaatttaaaatatattaccttgttatgttttttaaggagggaaatggcaattaaaattaaaaagcaactagaatctgcagaaatggtagatttgagaagtgatgctaatttcctcttcacatctaaattaactatctgcagtgcctttaatgtttcatttcttaaaacactattattaaaactgcattttccccncatgttccatatatgagtggaagaaaatcccttctgatactccacattagtctagaagaagagaagcaaggtctgctatattcctatcttcaataaatcatgtagtgtcttctcagagggacatattatgactaatcccagtagtcaccctcatagtctttaattagaagttaatcttctattgcaaattttaattccagctgcaatctcctttacaaaatgaaaattcttgttcataggacatgtaaaggaaataacatgctgtctttatgtgtgtgttttataatattgttatttggaatagtaaattcatagaatgcatgtgagagattaaaagatcaaataaatgtgttaatatactaatattgatcattaattttattaatattaaagtaacatattgatttatttgcataagcacacatagatcaattgcttggtatgaaatctcaatttctcttttgccatgtggcctaaagcataaaggaataaatgatattttaacttttaaattttagtttaattaggttaataatatgtaagttgttattttataatatgccatgtacattaaataaattttatcttaaaaaatacaacatacaaaaaaattcagccttttgtgtacatagaaagtctttaaataataatggttttaaaataatatcaaatatagataataaagaaataatgccactcacaaacattcaaaatcataacatgtttttataattgtttaagattgctttggtctactgtgttctagttggtatgtttcaaatgcattcttaaagtaaaatgtcattcttgaccttatggggcaccttagtgtatatgatagtggcaaaaattcatacactgacaaatatgtgaaataatttagaataaattatagttctgaatttcaatttcaatgtttctttgtttctctaggtggcttttacagtactttttgatttggaagcacttctgaagatatggtgtttgggatttactggatatattagctcatctctccacaaattcgaactactactcgtaattggaactactcttcatgtatacccagatctttatcattcacaattcacgtactttcaggtaataaaaataatgcctatttgtctttagaaatttatttatttccagagtaaatacctgtcacattattttaacaaccacatagaattatatataatatttatatataataactattatggtaaatatgaacttgagaaaggcacaatgacatcttacaaattagaaaagtgcattcacaaattgtaacaatcgctttttaaaactcctgaaaattcacagaaacaatgtagatgttttacttacttnttcctaaattagcagtatatagatacacatatttttatatatggataacatatatccatacatgcgtatnnaanttttaaactacataagatgttggnncaaactttaaagactaggaaacattaatttaatcccaagtgtttctcttccttgtcgataaggctaagtgtttgctcaacaacggtttataatgaaaatattaaaggtttataaaacttacctttacagattgtaattactcatacagtattatttataaatgcctgttacttcatgatggcacttataattcttatttaacttgtaataattggatatctttttttaaatgtgatatattggatcacattgctttctaattatgcaataaacagtaattattagttacattctttgttattattgaatatagcaacacgctcactgtcctggtaaatacttagtcaaaatgataaaatatgtctacatggtattctagtcaaaaacacctcaaatgtagtcattttcctgattcaaacttggtcatttattttttattttattttattttattttatattattttattttttattttattttattttattattattatactttaagttttagggtacatgtgcacaatgtgcaggttagttacatatgtatacatgtgccatgctggtgtgctgcacccactaactcgtcatctagcattaggtatatctcctaaagctatccctgccccctccccccaccccacaacaggccccagagtgtgatgttccccttcctgtgtccatgtgttctcattgttcaattcccacctatgagtgagaatatgcggtgtttggttttttgttcttgcgatagtttactgagaatgatgatttccaatttcatccacgtcccctacaaaggacatgaactcatcattttttatggctgcatagtattccatggtgtatatgcgccacattttcttaatccagtctatcattgttggacatttgggttggttccaagtctttcctattgtgaagagtgccgcaataaacatacgtgtgcatgtgtctttatagcagcatgatttatagtcctttgggtatatacccagtaatgggatggctgggtcaaatggtatttctagttctagatccctgaggaatcgccacactgacttccacaagagttgaactagtttacagtcccaccaacagtgtaaaagtgttcctatttctccacatcctctccagcacctgttgtttcctgactttttaatgattgccattctaactggtgtgagatggtatctcattgtggttttcatttgcgtttctctgatggccagtgatggtgagcatttttccatgtgttttttggctgcataaatgtcttcttttgagaagtgtctgttcatgtccttcgcatttatataatttatcatggaaacactagaatgtgcattgattgttttctaaatagatattgttaaatatttgggaaataaaatgacagtagaattttaaagaagtaattttgagaaacaggactctaaccccttaggaaaaagaacatgcattgcaactttattgattttatttattagtaataccaaaatattattttcagaacaatattcactttaactgccagatcacaggtaactagacacttttagtacctgggggaaaggacatgcaaatataattcacaaaaagtaaaaagcaaaaccccctacaaacccaggcaaaaagctgagcccttgtaatcacagctaagcatattgaaaccacagagggttttcatagttcaagcgctcacttccctcagcatccgtggatgtaaatctctcctcatktgttgagttatatcctgaagcttrgctatcataagtgacatggctggggcaagagatatacatgtttaaaactcttgatacaaaatgtgctttccaaacattcctgtgattataagctcttgtgcactgctctctagattcacaaagttatcatggccattgagtacctaccttagggcatcattgcacataattatagagaatatcctttatatcaaaacaacagcataccttttttacaaagctattaaagtagccaaattaagaaaattggtgttgcctatttcagcaaaggtacagtataaaatatattcagggccttaaaattgctatagtctctgacatgccatttttcagtaaaaagttagaaaataacatttgtgtgcaaaatatttattatagaattaatgagagaaaaaagaaaactgtttaaatgacaaacatttgaggaatagcttaataagtcataacgcgaatttaaccataaaattataattgtgatgagttttttataacttagaaatatgactgttttatttaaacgataaacagcataaattatattatatacataactacatatatgtatgtatatatttattatatacatatgtaaaatacctataaaatggaaaaaaatgactagaaagaaaccaatttattacaaattattaatcatctctagtttcctactcttgtcatcatgtctgattttcctactagtctgtttttttacaaaatatctaatattagcctgcaaggctgtaaccattttatagatagacagatgatagatagatagataggtagatgatagatagatagatagtagatagatagatagtagatagatagtcaaatcacagtgaagtttgcaatttcnagttnngncnttgattttggattattctgtaannngtnctgtttaatttatgttaatgattaggtgattttcatttgtatctctctatacatggccatgcttctctttttctaatgtattaataatgaataaaaatgatctcagttaatttgtggctagaaagtaaatctcttgagaaacttaggtgtcacaagggtttttgcaaatttgcatgtggagaatccactgtaccagacagagcggctctttgtataaaggcctacattggtcttccctattggtcctcacacttctcatttcaatcaacctttctttactgtgttggcaacaagtctttccttagacaatttcatatttctcacatttaaatgaagtaatgtttagtgcaaagtggcttcctttaaaatattagttatctgttagactaactactgaagttattcatgtatttattcaagtgatatttctgagcttctactatgtgtcagccactgttccagatactgaagatggagcagtggacaaagcagagaggattcccattttatgaagctgaaattctaaaggcaaacagttcacaaagaagtacacaaaggaatgtgaagtacaagtactggaaatgtcttaaggaagaagtgtgcagggtaaaaagcatagagattgtgtagtaggggctattttagattagttgttcatagaagccctctctgaagaggtgaggtttgagcagagactgaatgaagtgaaggagcaaactgtaaggaggtctgaggggaagcattccaggtggagagaatcacactgagcatgtcctgaggtgggtgcatgattgatgtgttcaaggaatagcacaaaggccagcgtggctgggctgcaaccaatgcagggcagcatggcctgtgggaaatcatcaggtcttgcaaaacgctgtggttcttacatatcggtatcatcctgatgatattgactggtgcgtcttgtgaatttcctgtgggtcgggcatcttgctgagtgcattgtctcatttgtttctcataacaagccctaagataaggactgtatgtgtaaattgaggtctcaagaaatctgttgacttgcccaagaccgcatacctagcagatgatggagctaaatcttcttcccggctgggcgcagtggctcacacctgtaatcccagcactttgggaggccgaggcgggtggatcacgaggtcaggagatcaagactatcccggctaacatggtgaaaccccgtctctactaaaaatacaaaaaattagccaggcgtggtggtgggcgcctgtagtcccagctacttgggaggctgaggcaggagaatggtgtgaacccgggaggtggagcttgcagtgagctgagattgcgccactgctctccagcctgggccacagagggagactccgtctcacaaaaaaaaaaaaaaaaattcttcccaagaacatgaatttcagagttcatattcttaactatgcaatttctacagcatctcactacaaatcaacagagtgacctttaagagaaggactcagaaatgtgaccaggaaaatgtgtttcagagacaaaatgttctgaagtgcttatctttgagagtatggtttacttcatgttgtatctgtaggaacacaaaatcaaaaatctttatagggtagatagagtgaaaaacctgctgatccctgaataaatgaaaaggcacaattagactcactgacagggatttttcctttctaggaatgcaggtttaaccctttgaaaatgctgatcaaatcaccaatgtggaagtggtattaaatagttccatttttttcaagctgtttacagttaggtactgaattatttatgtccgaaaatagacaaatctgataaaaatgtgaaatctttccagattattattctggaaattaatacgaatgttaatactttaatagttcttatgataaagctgtttgcttgttctttgtaaagattatctgagagtaatatgtcctgatttgggaatgtatgttaagaattacagcaggatggccgggtgcagtggctcacacctgtaatcccagcactttgggaggccgaggcaggcggatcacgaggtcaggagatcgagaccatcctggctaacatggtgaaaccccgtctctactaaaattacaaaaaaattagctgggtgtggtggtgggcgcctgtagtcccagctactcgggaggctgaggcaggagaatggtgtgaacccgggaggtggagcttgcagtgagccgagatcgctccactacactccagcctgggcgacagagtgagactccgtcacaaaaaaaaaaaaagaaaagaaaagaattacagcaggaaaaattggtagctgcaccatgtggtatgatgcttaatttgaacaacataatttcttacatatgtttaaaaattgcaaattagcatgaaggaaacatgctttagtcattttttttcaattggtttacttacatgctgtaaaccatatcatttgcattctgcagacaataggaataagactgaccactgatacttcctgttaaatcaaagtaaatgcagtaaacgtggtttcaggtatggcaaaattcagaggccgatatggtgtcaggggctcagtgtatgtcttcacctacctttcttctgttggtgtgtcggtgtcattcttaagctggttttctctatgtgaaggcaaagatggctcccaggagctctgtaagagtttgtgtgactcttacagcagtacccgttggggattaaaaaaaaaaaaaaaagaaatagaacatttcttattaaaggcatccagcaaaagccctgaggatgactcagatgactgtgaatactctggccagatctggatcatgacgggtggggagtccttcttgcacctttgtactaaggctggggtaggaatgatttgctaaaggaaaaccaggcagagttgaagaagccagaggcactggaaagccaaaaagaaacaaatgttattttagacatgtaaaaattaaaatctaatttttaaaaatgaattttctgtttcttactgtgttctaaatacactattcatctttaacagacaactcaatctcttattgacatcaaaaaatcttggatgatcattgtataaactatatttcatttatgccaagaatgcaattttcctatgttaatatctctgaatttgggagctgttataatcagtggtgtgttatactttaattggcagcatttttttatttcttagagctacatacaaaattggtgacatcttagacttaatgaaaagttgtatttttcacactatatacttcagcttttaattatgtactaatttatacatactaatcatataaatatgtccttaaaagctgtgtctttgtgttatactcgctttgtaatagtagccattgaatcctatttgtccaacacaaagtttggcacatagtaatcaataaaggtttgttaaacaaatgagtgaataaatttgtcaattaattacttatttttttttgcaaatgtgagtatgcaaaacatctaaatatatctgtatatatttagatgtgtgtgtgtgtgtgtatacacagtcatgcatcatttaacagcagagatacattttgagaaatgcattgttaggcaattttgttgtcgtgtgatcatagagtttacttacacaaacctagatgtatagcccactatatacctaggctatatggtatggcctattgctcctaggctacaaacctgtacagtgtatgactacattgaatactgtaggcaattgtcacacaaaggtaagtatttgtatatctaaacatagctaaatatagaaaaggtaagtcttataggaccaccatcctatacgtggcccatacttgactcaaaagttatgcgatgcatgactaaaatatatgtaaatatatacacacacacatatacatgcgtgtatatatagagagagaatagttgagtacatataacaaagatccaaaaatacaaaacttacataagacagaagtttatttctctgcagcagttggtaagtatgcagggaaagcaggtggctgtgccccccatggtcacccagcctgtcgggttggacaggacctgtcgtattctatgcaagccttctatttctggtccaaaggggctacactatttgtttccatttccaggcaagagaaaggtattttaaaaaggattcagagaaagtttaacttaatgcaccaaaacttgtacaaatcatttcctctcacattccgtaggtctgaacccagtccactggagacagctagctgcaagggagactggaatgtgtaggctatagccccaggcaaccacttgctaggttagaattcggaaagttctgttttttaaaggaggaaagagaaaatggatactgggctgataatctcaataagaagtaaagattcttggcaaaagtttgcttctctttccatttccttgtgattttctaataatacctaggaaaatattttatgagacagtgaatataactaatacaggttgagtatctgtatcccaaatgcttgggaacagaagtgttttggctttcgatgtttgtcagattttggaatatttgtatgtatgtgcataatgagatatcctgggaatgggacccaagtctaaacacaaaaattgtttatgttttataaacacctcaaatacattccctgaaggtagttgtatgcaatattcttaatagtttttttatgcatgaaacaaagtgtgtatacattgaaccatcataaagcaaaggtgtcaggtgtagaatttttcacttatggcctcatatcagcactcaaaaactttaaatttttgagcattttatgttttaaattaggaatgttcaacctgtattgaattaaaggtttccttgcatatttgaacgttagaaaaatattaattttatttcaacaatacgtactagtgaatatcattttgtataatcatataatatgttaaaacacttagttcatccattcatttgggatttattgagctagaccaatggttattctattgttaattttagtcactagatattcaaatagagaggtatgattttcataaagcactataaaataatattttagtatgaatatatttaagattcagtgtaaataaatgatcatatgtgtaaataaatgtttctctctcccttgataggttctccgagtagttcggctgattaagatttcacctgcattagaagactttgtgtacaagatatttggtcctggaaaaaagcttgggagtttggttgtatttactgccagcctcttgattgttatgtcagcaattagtttgcagatgttctgctttgtygaagaactggacagatttactacgtttccgagggtaagagttttaaaaatgcagtaagttaaattcattgttcttattttagtaataatgattaacatcaaagtaatttcactttagtcattcagaagtattatcctatttttgataacttgggataattagattttaatatttaaaattgtctcttctgcagggtgcaggtgtttgagaccattttcagcattgtcagattatatatcaattctaaaattctggaaataatctaatagagttagatgctgaaacctgaatgctagggttcttagcacctataaattaaaatacagcagaattttaaaataaagcttgcatttttgtgttcaagagagttaacatatttttaatgttttataatttatatattgttatgttagtcagaatctagaaggaagcttttgattttcaataaaataaaattcataggctcttcagattttaaatgtctctcagctttctcagtagaattcacttaagctaaaaaggaagttagaaatcaatggatccataaacaaaagtcatgtaagaggtacagctcttaaccagaaggaccttgaaatttaactctatgtggacttagaaacataaaaaaatacaatgaccagacaaataatgtatatatttttaccctaagttgaaatagtaaggcctattatatggtgtattagtccattcttacactgatgataaagacatactcgatgaaactgggtaatttataaagaaaaagaggtttaatggactcacagttccacgtggctggagaggcctcacagtcatggtggaaggtgaaaggcacatcttacatggtggcaggcaagagagaatgagagccaagctaaagggggaaacctcttataaaaccatcagatctcgtgagacttattcactaccacaagagcagcaggggggaaaccgcccccatgattcaattatctcacacctggtcccttccacaacacatgggaattatgggagctacaattcaagatgacatttgggtggggacacagccaaaccatatcatatgggaagaataatattggggtaaaaacaacatatatttttaaaagttaagctttgacagcaaaagggtctgtacttctaatggatagcttagctttattttagggatttttttaaaaatcctatgctaaatttattattttagctaccagtgttggaattcatccttttataatgcagattcttcattcatgtgccgccaaggatttgtcgagtgcctttaaagcatctggtcatttaggtaatcatggggacataagtcctcaagaaattctcagttgaggggaggaaccagtaggtaagcacagggcaccacaggccttgatgaaggcagacaccaggtgctctggggacacagaggcaaggccgattcccaggcatggaaaagtccagaaaggcttatgggaacaggtgactccaatggctggatgtagccaaaggaagtgcaagggaacggattcccatcacaggaacagcatgtactaaatgtctgaggtgagcaaatccttcttccagaacttagcaagaaccatcaatggtaagaggaggacaaaaggccaaggaactctgaagtcggttgaaagtgtgatcgtatctgtcatgccaaactcctatcaactctagtagggaaggcaccaggttcaagaggctgaagaagagacccagagccagcagatgagacatggggtttcattgagggcttacatacaggggagggagtccaatgctggctggctggtcaggagaacaagacctgcttgcaaaatgcatgcaatttacatagcatttttacttagcacactcccccagcaacctccacatggaaatcttaattcaccccagacttggggcctcaattccctgtgtatagccagtgttccacgggaggagatggggactcagatgttcctcatcaacaaggagtcagtctctgggttggccactcctagattccctagctcagaacacacattcaggtgtgtctggcaagaagggtcattctcggggtatgcttgagttattgctttcaggtgcacttacatacgatgtttaagtgctgatgagagagggcaagtagctagtgaggttgcagatgggtgagataaggaggatgatggcaaaggttcctgcagaggtgggagaaggcagtatccacagcaagaggacaaagattggccttagacaggtgttgagctgacaggtgtcgaggtgatcctcaggacagcaagccaagctgaaagttacaatcaagactttgttccctcactgcaacagtacaggcaggaggcaaaggtaggcaccgctctccagctctccagcgtccggaggtgttggttaagggtcaggtggatcagcaggcatgggggagggagggctcttttccttgctaagcagtcctgaacaaaaggttccagccattcctgcacctagggaggggaagcataaggaaggaagggataggagaagaaaggaacggagccagcatggaggaaagtgcctcatctgggccctgatcaggaggcctctgcacagaggcacctggtctgggactgcagctatggccatgatcccagctggcctggcagggcagagctgagtcataaattgcaagtccctccagaaatggcagtgccctggtcatgcaccatgtctagggtggggatggcagtggtccccgggaggtctcccaggcaaggccttgccatcaggtctgaatggatcacacataggattttggccaggagcaggactcctggaacggaggctggagggaggcaagccaaaatgatagccacatcattaggcccaccttctctgggtctgccccttcactgggtaaataaaaagatcagctggccatcttttccatgggaacatggggatggttattgtaagagttaaagaggtaagaaacacaaaaagcagctcaacagtaaaagacagatttattttggagaataaactttagaggggcttctggccaattttgctcaggagcattctctcttacagactaagggtatttaagggtgtaggaagtggggagcttatcgcaggttcggaatgtttctatgtgagggaaagtttattgtggggttgggattgggatgtctggttggaggggaggctgtctcagggttggcgtgtttctggtcagagggggtttatcttagggttggaatgtttctggtgatgctgacattaggcattaggctgatgtttgggngcttgatttgggtgtttttttaatcaaagggaacttaaaatggtggtgtttctccaagacgacgatctcctgctctgtcaatccagtccctacagttatagaaaggaagaggggtggtatattctttctggctacttcctgctgagtggagggtcagagggttctttggtcttgggttgattgtgggagtaatgccatctgtagatgtttttaggtaattgtgagatgtccatgatcctgtcagttaaaaaatctttgaaaaaggtgaattaagccaggtaagcacattagtcctaggcatattattaggagagggcccaagaatgggatggtccatgttaggattttgtttctaaaccaagaatgtatttggttgttttggtattcctttcacgttttagccctttcttcaagtttttcagcagcatctcttactaggcctgattggttgagatagaagcaacattcctcactcagtgagaggcagagaccccctttttcagccattgtaagatctaatcccagtctattttggaggactgctccaggcaaggagtctaattggtcttcgactcttataaggctttgggctatatcttttaataaatcctgtagttctgttgaaagaactttaaagtatgttaaggagttggccagtctgcctgctcccaatccaagcccggaggttatacataaggcagccattaaaggaatgacttggatgctcctcctttttctaacatactggatgaatggaacaggtgaagattgattaggaggaactagtccaacggagggagaaagataaactagggtacaggttctggtctagttggtggggagaaaaagatatgtgttggtaccgcataaaaagaacgagcctttgtcataatgcaggcggagatagggaaagaagataagcaaattaaagatggggtgttttttctttcctgcggttttttactccaggtggacaaggaggaggccagggagacacccactatagtagagatataggaagagttattttttacacatttaatgcaatcggatgttgcaccattgatattgagccatggaaaaagttgggcaccaggggcagcgcaagttaggcaggaggcattggttgacggagaggctgtaaactagctggttgcagaaatgctccatttgtttcaggtgatacttggtagtcaacgtggctactttgatggtcagaggggtgtttaacaatgatagtgtgattgcattggttagatgttaaggaccctacagggaaatttccctccgaggctgaaaagcaaagtgaggcttgttgtaaaaggggggtgtgtgtagttatgggcccttcaatgggagggtcttggtgcttaaggcattgtagagagttgtaataggtttgaaatattttgttggcccgattggccctggaagtgaaataatctctgaacagggtgtcggctctctcaaaaaaggaagctccttttgggagtttataagttagggttatatttcctgttcaaaggtcatgaagaggtgtaggaagggctgtgtaagctgaggaagacagcgataagcatatccagcactctggagcaaaggaagagttagctttgcagcaagagggattgtgtaaggtttatagagcattctagtttgagagcagtgaggagtggttttattggtgaggttgatatgattagggaatttatattgaaagaaacaatcaaaaagagaaaaaagttatttgggtagtagtaaagtcctggaaagttccttgaagccataggtaccatagaacagtcagaaactgattagcatgtataatgggagctttgtaagggtaccactgaaggtctgtagcaaggttggttaggaagcagtgaaagtttgggagagaaagagacataagcagcttatgtggatttctcttccttttcctccaggatttaggtgaggcgaagggaagtaggtcctgtggagacacaagagaaggctgaaggggttttagatttggttgtttgtgtatgtggtgaagggaagtctgttttcttgagagaggtataatgaaaccagttggggagtccctggagttttgctgccatgggtgtagtaaggatgatctggtaaggtcctttccatttaggtgtaaggagggaattggggctaggactgggatcttttatcagaacccagtctcctggctgtaggaagggattagaggagttggtgacaagttgtggcaggtgtttgtcagcatattcccaaatgaaatggcagatggtatgtacaagaggggaaatgagaggggttggcagaggtggggcttgaccctgaggtggaccaaaaggggcaagtggtctcccatatgtgagttcaaaggggctgagcattaaaggtttatgtgagagcacctgaatttttagaagggctaaaggcaaaagtgtaaccgagtctttatgtgtctggagtgagtacctggtgagggtgttttttagaatgccattcattttttcaaccttttctgaagattgaggtcgataggggatgtgtagcttccaggtaatttgtagggcttgtgaaagtgtttgagtaatctgagaaatgaattcagggccattatcagattgaaaagaaagaggcaccccaaacctggggatgatttctgttattaatttggaggtgacattagaggctcgtttgttggttgtgggaaaagcctcaagccattccaaaaaggtatcaaccagaactaaaagaaatcaaaccctttttactgggggtatatgggtaaaatcaatttgccaatcctgtcctggaaggtgtcccctggcttgatggattgggaaagaagagtgtctagtgttggaatggggtgaagctttctggcaaatagagcattgatgggaaatggcttttaactgttcctttatatctggggttatgtgtttatgggaatttaagaagtgttgtagaggtgaatggctagcatggaagaggttgtgaatgtcccgtaaaagagttgtttttttcagggtcaggtaggactaatttgttttgtatgaatcagtttggggggtttgaattgtgcccctgccatgattagttgttgtgtttggtgttctggataaaagaaggggatatgttgtatgaagggaaataagtattggggaattgggtgattggttgagacatgttttgcccagtagtcagcctcatggttccctaaagaaatgtggcttttgtctgatgttctttgccatgaataactgcaaccttttctggaagtagaagtgcctttaatagaggatgaatgagctttccattaatgataggggttcctataactgtgagatagccccgctccctccaaatttaggcattggaatggatgatgttataagcatatttagaattggtgtattattaacttgtgtgttttttggtagggttagtgctcttattagggcaactaattctgcttgttgggcggatgtgcccaaaggcaagggggcagcctctatcactcttctagatgggagagagtgggtatcataacgctatccctcaatgatggcatatcctgcttggagaggagggttttttgatgcactgccatctataaaccagtctggggctccctttatgtgagtggaagtaagatggtgaaacatggtaggaggactttcaattagatcagagcatgagtattggtcaggatctaaaatcggtattgagggtaaaagagaggcgggattaggtggggagcatctatgaagagagatagcgggttgaaggagggttaaatgtaaggcttgcatatgagaggatgagatgggggtgagcgcctatggctgaccatatcttgtagactgtgagaagaaaatacctgaaggggtttgtagaacatgagtttttgtgtctcagagataattagagaggctgtggccaaaatttttaagaaaagggaccagattttataaatggggtctagttgttttgaaaaatatgccactgttgagaaggagtctcccataagttgggctaatagtccaagggcctgattatgagaactgtgtaaatatagatgaaaaggtcttaggaggtttggaatgtctaaagcatgggcctgcaataaggcacgttttagatgagaaaaagcatgataaaggtctggggtgggagtgagtggttggtcaagatttccttttgtgtgttcataaaggggtttagcaataatggcaaaatttgctacccataatcggaaatatcccactaatccaaggaaggaaagtaagtcccttttcgttttaggaaatgggatttgttgaacaccttgctttcatgccaacagcatttctcgggtatttggagttatgatcagtcctaggtatgaaactttcggtgaggttaattgggccttccttttggacaccaggtacccacattcagccaaaacatttaaaatcttggtggtgtgttgaatacaatggtcaagaaaggggctgcaaaggagtaaactgtccacatattgcagcaaaatcctgggttgtagaggtagttgggagaggtctaattgaagtgactgaaatagtgatggctgtctctaaacccccaggggaggacagtccagatgagttgttgggagtagcctgtgtcagggttggtccaagtgaaagcataaaggttttgagaggagggatgtagagggatagtaaaaaaggtgtctttggccaggtgcagggctcatgcctgtaatcccagcactttgagaggctgaggtgggcggatcatgaggtcaggagatggagaccatcctggccaacatggtggaaccctgtctctactaaaaacacaaaaattagctgggtgtggtggcgtgtgcctgtaatcccagctactcaggaggctgaggcaggagaatcatttgaaccagggagtcagaggttgcagtagattttgctactaaatgtactataagaaaaaaaaaaaattcaaaaggaccttcaagtattttcagaaatttttaaattttggagttgtgaataaggactgtgggcctctatttttaaaaaatgcatttaagagtctgtacttctgtccctacaagtccgattgtgcatcaatgccccattgctttttagctatgtgatactgggcaaatcattctttacatttttattcttcacaaccaagatgtaagccgaaatgtgtttttttttcttctgcattttgtgtattaaataatatacaaagatgacacaactgggcttagaaagtatttcttaaaagactagattacagcctatgtaatatatttgaaattgataatattatctgtataaatggcaaggctgctttttagatccattcctgacttagcccctctatacttgctctattttccagctataatagtaatcaagtagtattacattttcagatctccaattactcaattgcaccattttgcctgattattttgaccatcataatgaaagcagctcggcctctcccaggccccctaatcagttattcagcagctgacaatggtgacagcctgaacgtgtgaagtgactagaaggagtatatttttcctttcacataaatgttactaaaggtgaaatccatgaagagtaataaaattttacaaggaaatgttaaaaaatattacaattacaaaaggggtcattatcatccctaatcaaataaagcatcaccaattggtcttacttgccagatagaaaatcattcaggagagatgattaaaaacatattttactcaaggctagatttgtcgtcagacgcacatccctgggtttcttttccctcccttctctagacattatccagtggttgagtgcttaaggtaggttggttaattacagtattagccaaggcacaggacatgaccagaacaaattttctgcaagaaaaatcacatgatgtatcgcaaagtgaaatacagactttaaaaatcagcaattgtctatgttatcttgacttgggtccaggccgtcttggaggctgcctcccagcaatgtttatagggctgcagaatggtgtgggaaaataaagtgtggaagcacatgggaggcttggggagggcactcaccctgaatctgcttcagcactatttaccaggtgtgtagtcttaggtgacacttaatttgtctgagcttcattcatttttaaaatggtaatgatgtctatcaaacaggcatgataactaagagccctacttacacatagtaagactttgaagcttggagagcttgcataacccacacaaagttacacagctggacagctggatttgaagttgtctactgctcctaccaactattatccattaggtcacttctagccaagcaaacacactagaaagtgttcagttaggaaatacttattgagtgccaagaatataaatattttgtcaatctgtacctggaaggaaaggcctctggaatagttcatcacaaacttcccagaggacttcaggaaatcaagaaaggtatagaggtattaacattttggggcacagcctgtgagtgttaggaatgatcatgtcaggatcccaaaccagcacctgatagtctctgtgccacagcagaaacagcatttgtctaagaagatcccacacatgagacaggtgaaaggatgttccaagtgacagcttgtgaagaggtgtttgaatggagaatgagcaacaatgctgaaccaacacaaacatctgtcatccttacatactgggtgaggctcagcatgccggattccttttcccagtattgttcattcctccaggatcctcattaccctccatcattccccttaccctatccaatcagaagcgtctgcataattccgttgcaaacactttggctgcaagtaacagaaaacttgattccagttgactttaagaataaggaaatttattccataaaattcaagagatcaggtagctttcaggaagtcaggactggatctttcactgaaattctcttaattgttgtttgaattgtcagtgttcccccaaaaatgtatgttgatatcccaactcccagaacctcaaaatataactatatttgaaatacaggtaatcaagttaaaatgaagtaatcagggtggaccctaatccagtatgcctgatgcttttatgaaaagggaaagaacagacacaaatatgcataagaggaagatgatatagacccacagtatattagcctgttctcacattgctataaagaaatacctgagactgggtaattacaaagaagagaggtttaattgctcccggttctgcaggttgtataggaagcatggcagcatctgcttggctcctggggaaaccacaagaaacttacaatcacagcagaaggcaaaggggaagcaggcaagtcttatatggctggagcaggaggaaaagaaagagagaggaggtgctacacacttttaagcaacctgatttcatgatgactcactcagtgtcatgagaacagcactgagaagatggtgctcaaccattcctgagaaaccacccctataatctaatcactcccatcaggccccacctccaatattggggattacaattgaacatgagatttgggtggggacacagatctaaactgtatgacacggggatatggccatgggactggaatggcatctacaagccaaggaatgtcaaagaatgctggcaaacaccagaagccgtaagatacaagggaagatttttttccctagagctgtcagagcacagccctgctaacaccttaattttagacttctggcctccatacctgtgagacaatacatttctgttgttttaagccaccaggtttgtggagcttttttaccacagccctagcaaactcatacagtgaccttctccatgggttgtcttcctatgttagcaaaatggatgtgacatttccaggcttcctgatgacatctagaggaatatgtgggtcctctgttcccaggcctcctccttaggaccaggaaaccttttctatcatccctttccttccagcaaacttgctgtcatctctcattggcaggattgagtaacagggtcattcctagaattaaaattactggctttggttaatcacatgggaaggagtagatgttgtgaagacaactacttgggtcagacccaaaacctggacaagatttctctcagctctgtctccaaagctgctcctatattccccatgcaaacaaggtttgtaaatagacgtggtagaaagaaagctggtgtttaattcctttcaccagaaacatctggaccccagatccttcccaagatgcccaaataggtaggtacctgcaaagtagctagcatagggctggacacagagtagatcagttatggattcctgctctccctgtttttatggtactggtcagtttctatcacataccattgacattacacatgggtcttactgcctttgctagtctacaggtaccttgaggttgagagcatgtgcattccttacacctccaagagcacttaacactgtgcagtgggatctgtaagcatcagtaagtagagtcagaaaggaagacagctaggactctatcagtatataagcccaagagatggagaagggatgctcgaattctactcgatggaaaaacaaagaatgctatattgatagaatttaataaaacaactttgtcatctcaagccaataactttctgttctagtttggaccaaacagctacactgctaaatagaaataacaagatgaaatcctgatgtaaggcaatatgcaacagttcctgttttgttgggatgaatatattgtatgagtacaatttagatgaactattcattctggaacagcctgtacaggtcctaagacagattgccataagcaagaacaaccacagtctttctgtatcaaaaacaaattgcatgtttttccataattgcctaacaaaagctcataagcagcttcagtaagattttactcagtttttcctccatttattatagaaagcaactgcctctaaattactgaaataaatgcagtaaacattcactgtccatctttaaccagctctgtatctttattccttctgatttcccactaaaataataaacagacctcatatgtttagcggttgtataaatggggatagtttttatttctaagtaatatattatcctctataacacaaagtacttaaattttagtatgatttatttgatataggttttcttaattttcattctctaaatgttaattacttgctctattgagttttcacttataaaatatgtccaaaacagttattgcacgatagaagcaacaaatgctataaaacacataattaaaaattaactttcctttcctcttgccaccaaaatgactagtcgtgcagttagttgtattaaaggaattaccaaaaaatttgaagaaacaatttgctccttaattttttaaattatttgtcagatgtagtgagagtgtgtgacatttttaatagattcaaggtttattttaatatttaaaactattagttcaattaatattgctgaaatctttaatttttgttctgtctttcatgtatttttaattttatcatgaaatatttgatgtgcaaaaagatatgaataataataaaacatactcctctgtggccatacttaacaaacactaaattgcaatagacctgaagtccctgggtgcctcttcctgatctctgtcctctcactctcccatggagaatcacttttctgattttgatgtgtgttttgttaataaatttctttatacttttaatgcatatatggacatcaactgaaaatcatatatcttttgtgtaatttaaacttgccttatatggtctcattctgtactatttctcctgctaattgcttttattgctcaatattatgtttatgagagttacctaaatgagtacatggagctgtattttatttattgtcattactgtataaaaagcaactacatattatattgcatatataatacattctttatacagacagaacacaatttattttttcattttcctgtgcagtaggcagattctaagctgtcccccatgatatctccgcacgagtgtccataccctgcatcatcccctacccttcgatactggtaggatttgtgaatatgatgggctgtcattcctgtgaataggttgctaagcatttaatgttgagtcagtcataaatagagattgtcatgtgtgggcctgtcctaatcagaggtactggccttttcttagatttaaagcaagaaagggtcttctgttggcttgcattaggagatatacctaatgtaaatgatgagttaacgggtgcagcacaccaacgtggcacatgtatacatatgtaacaaacctgcacgttgtgcacatgtaccctagaacttaaagtataataaattaataaataaattaataaataggaaaaaaaaagtaagcctccatgttgtgaagtgctgtgtggccaagaccctgggcaacagccatcaagaaagcaggactctcagtcctgcaaccctaaggaacaaccctctgataataacccaaatgaagttagaagaagaccgcaagctccagatgagaccaaagcccagccagcatcttgatttccaccttttgagactctgagctgagagcacagttatattacaccatgcccacattctgaccttcaaaaactgtgaagtaacaaatgagtgttaagctgcttcgtttgtggtaatttgttacacatcaatagaaaagtaatgcatccaggtgttggatgtttctaaatattgtaaatccaaacagtactgctctgaacatttttgtacattgatttgtgcacatgtgaaataattctttttagggtatgtatctgggaatagagttgctgggtcttagaataaacatatcctcatttctcattggtattgataaactttcattccaaagtagttttaccaatttatcctaagctatcagcagtatatgagagtattctctactttaccactcaggaacacttggtctggtcaggctttattttgcttgaaacacttagctcttcatttagcctgaattgatttttgtggctgctgtgagagagggaacaattttatttccatttggataaacaattgccacagtaccatttattgaccaatctattcatgccccctagttctgcatcatatgccagctgtgtcataacgtggttccatatgtgagtgcccaagtcaagaggcacaggctcccttgtcatccccttttgccattgaattttttttactggatctggatcaacctcctctctgaggtttaaaaccaataactatatagctttgctcaagtatctcagcagtctcctcctttcaaaggttcatgactttgtaccttatctctgacacaacattaaaaaccaaagtctagcagagatcggaaattctctagagaaactgacttttcttgattcaagctacttcttagcttttggtccctggagtttttaattattttcttggactattcacctttaaaaggatgctttttgcattttatccagaatttcctggggtttttgaatcatttttttccatacaggatgagaaactgaggttcactaataaatttttgagaagaagaaacagtactccatggactctcagatggatagacttttttaaaaattatactttaagttctagggtacatgtgcataacgtgcaggtttgttacatatgtatacatgtgccatgttggtgtgctgcacccattaactcatcatttacattaggtatatctcctaatgctatccctcccacctccccccaccccatgacaggccctggagtgtgatattccccaccctgtgtccaaatgttctcattgttcaattcccacctatcagtgagaacatgcagtgtttggttttctgtccttgcgatagtttgctcagaatgatggtttccagcttcatccatgtccctacaaaggacatgaactcatccatttttatggctgcatagtattccatggtgtatacatgtgccacattttcttaatccagtctatcattgttggacacttgggttggttccaagtctttgctattgtgaatagtgccacaataaacatacgtgtgcatgtgtctttatagcagcatgatttataatcctttgggtatatacccagtaatgggagggctgggtcaaatggtatttctagttctagatccttgaggaatcgccacactgtcttccacaatgattgaactagtttacagtcccaccaacagtgtaaaagtgttcctatttctctatatgcctccagcacctgttgtttcctgactttttaatgatcgccattctaactggtgtgagatggcatctcattgtggttttgatttgcatttctctgatggccagtgatgatgagcattttttcatgtgtcttttggctgcataaatgtcttcttttgagaagtgtctgttcatatccttcgcccactttttgaaggagttgtttgattttttcttgtaaatttaagttctttgtagattctggatattagccctttgtcagatgggtagattgtaaaaattttctctcattctgtaggttgcctgttcactctgatggtagtttcttttgctgtgcagaggctctttagcttaattagatcccatttgtctattttggctcctgttgccattgcgttgggtgttttactcatgaagtccttgcccatgcctatatcctgaatggtattgcctagattttcttctagggtttttatggttttaggtctaacatttaagtttttaatccatcttgaattaatttttgtataaggtgtaaggaagggatccagtttcagctttctacatatggctagccagttttcccagcaccatttattaaatagggaatcctttccccatttcttgtttttgtcaggtttgtcaaagatcagatggttgtagatgtgtggtattatttctgagggctctgttctattccattggtctatatctctgttttggtaccagtaccatgctgttttggttactgtagccttatagtatagtttgaagtcaggtagcgtgatgcctccagctttgttcttttggcttaggattgtcttggcaatgcgggctcttttttggttccatatgaactttaaagtagttttttccaattctgtgaagaaagtcattggtagcttgatggcgatggcattgaatctataaattaccttgggcagtatggccattttcatggtattgatccttcctatcagatggataagacttcagatggatagactttttaaaatgagcatgatctagggcaggccatcccagtgtttgcctttactgtccgcagcctcacagtcttttgcctttgtttgcattgaggatattgtgtcctctttgaatgtcatagcctcagctttaccctccctcagtctgccacaccactgaaaatcatctttcacaaaatatctgatatacccatttatgatgaagataaataaatcatgttcttggtttttcattcataattctatcaacattctttatgcaacacagctaggaagcatactgacatgaacaaatgtactcactgccctctttaactatagcattgtcaagaacccaaggaaggactgaaacaactcatcccatagtagaacacataagaaagatctcaagtaagccaaaggtgacatatgctacaatagatgtactcagttctaaagtaatagcaaagacatttgggagagtattttaaaattctgggaatttactgtgtgcctagcacttccaaatatcagctcatttaatgagagaaaaatattgcttccagagaaggaggtgtgcagagctttataaagatttgtctttaggggtgaatataatttcaacaggtagagatttgggtcatggcattagatgaaggcattccatataagaaaaaccaaatgagtaaattagagacatggagaatcacaggcatggagggacatatgaccaaaggatggcagtagcaatgggaaaccagacatggagggtcttcagtggttggaactgtgacacaagggagcttttgaagatttttactttattttttttaattttttttttcttggagaatagcagaagagctattttgaggaaagttacactggaagaaatgttatttctatgaggaggtattttcattgaacaaaattcatttttattgatgaaagacaagaatttatgatcagttgataaatttcaggcatctttggagatattttatatttttttctgtgaggctgttactgtgtcaaatctttcacagtttcataaaattgagtctcatctgaagctttaggccagattgttatccatggttgagcatgactctgcaatagaaactaacaatttggcttattttaaagtgataccatgagatatatatatatatatatatagtttattgtcattgtctccccccccaaaaaaaactgtaaaattaattttcattatttcctttctactataacattagggtttttttttccttgctcctgttggaatttatttttgtggtgactcatctgtggcataatgatgaaatgataaaataatatcctgtgtacttccttttagaggatgattcacataaaattggccatcttttcctgctgttgtatattaatatcattggtccactgaactgcaatgatcagaattatgactttccttgaataaagcatcctgtttcctacccctaaccctagaagagtactctttcagggagaacctatttttgcagctcatcttattttggtagtttttcaccaatcatcttatttgcaatttcccaactatttaattgtcatcttacattaatattattttaataatcagactgttccattgttcaaattttaaaaataaatgaaagtcattaatgaaaagtattgtctcattccattataatgagcttcctcaagtatgttttcagtgatgaaaacatgaccgtgttattaaatattttgtggattcttgcctacaaaaagaggacagttcatacatttgtgtctgtatgccttttagctctactgcataaagaatgtcggtggaattttgaacaacaaaacaggcacatgatttatgtagtcttgcctacaaaaaatacagtctgtaatgagactctattgtcagtctctcatggctacccacaatttcctatcaataaaacaggataataatacataacttagaggattggtaggaagattaaaggaagtaacaggtgcaaagtagttagcactgcgcctgccatgtaaacaggcacttaacaagtagtagcagttgtccttcttaaatgcatggaaagcccatgtcttagcatggcatgctgaattcctagcagcactgaggtagcccagcatccaagggtaaatgactggagaattaatggaatggtgggcactcagttttctgtgttgtatagaaagtttaaaactttaaaaatagtccccaattccagtttaggagattatttttaatcagtcttttgcgactttccagacaaatgcaaaaggtaaaaaaaaatcatattacagaaaaataagacaaaccgacaagttatttccagacgcggaaatagcttatcattaactattacatatacggaactaaacagagaccattcacccaatgatatgtatattcattctttctcttgggtatacccttcttggatgtaacacccagagtcttagtgtgccagaagactaaaatgcctacttctgcagaggactgtctctaacattttttctaggaatcaatgttgaaatcttaatttccaggaagcatgatgggataaagcataatgtgtgtacgtgtgtgagcatgcatctggctgagcacatgtgcatgcagatgggggtggggaaggagagagggaccccccctaagcaaatggaagctaatgggctgcatcctataatcttaaattgtgcaatacaaaaggaaaagcagagcaggtgggaccagatgcagaaagaaccttagtagctagtttagatggaggtctgcagtattgctgctgtggcttttgctgttgcttctttgtgttgttattttagttttcagaattccggagaaaaaggcaacaggcaaaagataaaaatcctgcctcaatccaacagaaatgtacttgctaaattcctgtgagaactctgcaaagttggcaataaagaatgcatattaatatgaaccaccaaaaagtgaattctgttaacattctagacaaatgtcacatttgctgtctcttgggataaaccaagtcatttcaagttgagtaaaatgaagtaaagccagtgggattatgcaaaggtacagcaaatgaaaggaaaggcaacactctcttgaagagcgagggagaactttcctgtattactcactagacaaagattattatcttcatcttatagttgctgccaattcaacaataaataaaaagaaatgtttattatatttggccttgttctaacagaatataatgtgtccggcaataattatatccaggtcaaataaatttaatcaaaatttaaagtaattaaaaataaaacaaattaagatggaaacaaaaaatggcactccaaaacctaaaaggctcatcatgaggacactcatgtttcccagaatcccggaccaagcgtgactaatttttctagcaaatatacactttgatagacaaacatgtttatacaagttctacagtgattatgaggtaccaactagttcctagagaagcataattattcctgatcaagtccagaaagacatttttctggatgttattaatcttaatggttactatatgtgattgctttttatgtgtgagagtttacaagagccttgaagaaaaatggagagatggatgggtatacacagagagagagaggtgcacacacacatagatatccacatatatctaatataattctcaaaacgacttcattaaggaaaattatggacgttataaaaaagaagcagtgaggaacagcaccagaaattgatgagaaccaacaagaaaacatataaggctacaataactagaagtgcatgacttagggttcgcagacagatgaatgggcaaacacgcattgctataaagaaatacctgagactgagcaatttctaaagcaaagaggtttaattggctcaccattctgcaggctgtacaggaagtgtagcaccagcgtctgctccttgtgaggcctcagcaagcttccagtcatggcagaaggtgaaaggggagcaagcggtttcacatggcgagaacagaaacaaaagagagaggaggcgccaactctttcaaacaaccagatctcctgtgaatttagagcttagagcgagagctcactcgttatcatagacagcaccaagccatttgtgagacagctgccccgtgacctaaacacctcccagcaggccccacctccaacactggggatcacatttcaacatgggatttggaaaggacacacatgcaaaccatatcaccatatattcactcaaacaacatgtaacaaagtgtcacaatgaatgattaaaagacatattaaacaagttaagttgggaatactaaagccctctcatactgtgtccggaattgttgggttcttggtcttgctgacttcaagaatgaagccgcagacccttgcggtgagtgttacagttcttaaagatggtgtgtctagagtttgttccttctgatatttggacgtgtccggagtttctgccttctggtgggttcctggtctcgctggcctcaggagtgaagctgcagacctctgtgatgagtgttacagttcataaaggtggcacatctggagttgtttgttcctcccgtcgggagttctttgtccctcctggtgggttcgtggtcttgctggcttcaggagtgaagctgcagaccttcgtggtgagtgttacagttcataaaggtggcgcatctggagttgttcgttcctcctgttgggagttgtttgtccctcctggtgggttcgttgtcttgctggcttcaggagtgacgctgcagaccttcgtggtgagtgttacagttcataaaggcagcgcctctggagttgttcattccttccggtgggttcgtggtctcgctggcttcaggagtgaagccgcagacctttgcggtgagtgttgcagctcataaagacagtgcggacccaaagagtgagcagcagcaagatgtattgcaaagagcaaaagaacaaagcttccacagcacggaaggggacccaagcaggttgctgctgctggcttgggtggtctgcttttattcccttatctggccccacccacatcctgctgattggtccattttacagagcgctgattggtccattttacagagtgctgattggtgtgtttacaaatctttagctagacacagagtgctgattggtgcatttacaatcctttggctagacagaaaagttctccaagtccccactcaacccagaagcccagccagcttcatgtctcaatactatgtatcaaaacattttcttgatgaattagagcaaggatatttaaagaatgaaaaatgtagagaacaaaaagaaaataaaatcttaatcttggatagtcaaaggccacctaagcctaacagaataaaatcaattacaaagaagaatgaatttgaatttttaaacaagtcaaaaataaccaccaaaaatattttctaaacaaaatatgaaatacaaccttaacttaccatttcagtaagaaaatgtcaaatggacaagatttatttgctatatttggtttttgttatcaacaatgcacactgctggcaaggaagcacagatactgtgatatattgcatgtggcagtgaaaatttgtgtaacttttctgagaattggttttttattatataaaatgacactaaaaagcatcttatcctttgatcttctaattgcatttataaaaatctctcctanagaanagtaacctgagatgcatacaaagattgatcttcaaaaaatgttttttgttgcattatttactgttttgaaaattttgaaacagtttttctgtccaaccatagtagaatgactaagtaaattttgaaatatagtggtttatcatgcaattatcaaaactatatttttatagactaattagtgatgtaagtcatgagtgagagaaatggtgatgataaaatatttttaaaaattttaccaaactatatttgcagctgaattcaattttgattcaagtacatttgtatgcctagaaacaaactgatagaatgtttaaattacaaaacaaaatatatacattttttgttatatgtgtatatatatatatatatatttaaaaaacctgttgtttgaattattttatgcttcatatattttggtattttgagattttttactatgaaaatcttcaaaaatggaaacaggtggactgataaacaaatagatataaaaccttactttatagttaaacatcagtgagaaaaaaactgttttttaaatggcaagtacatgaacaggtaaagttctaaaagaaagaaaatacacacacacacggacatgctagttatatttacaataaaactaatttaggtttttgacttgtaaaatattttattaaagtaaaataatttcaattaagtgtttattcataaacagctatcaagtttatttttaaggtattaaagtactaaataagcactattagaagtgtggctttgaaatttatatctgcaaaagaccacatcatttttgataaagctctttatttgtgagcttagtgaagagaaatgagaaatcaaagttgattacacaggttactttttcacatgataagagaaagatacagggagggaggaaggcaggaattgnntgagattttagaaaatctcagatttaaaacttttaaaaattaagtattgggcatgaggaaatgagcagagaaaatagtagtcatatttccacatttgtaaagagaagaacaatagccaggcaaactccatgtctgaagtgtggtttattttaatatgcaaaacataagcatttctctgaccaaagctgctttctatttaaagctaaatataaaaatattatttttgagcatagtattgcgagaacatagtgaaatttcctatgtagattctgtgctgttttagttacggaaatatttttaattaatcctccatttaacaatggggcatacatctgtgtttttcgaagcactgttttgtatatattagtaattatcattgcactgaaatgaaaatatcaatttcatgtacaattttatagcacatatttgattgacaatgaaatgcatttaacttataaaaatgtaatatggctgacattagcaaaatctcttattaagaaataggtcataagtactccaagaaacttgacagtctgttctatgaagagcattaattcaaaagtcccctatttgcataaatctccaggtctcattttcacagctgtttgttgtcagctagacggttgcagttcaacattattttaacataatcggtgggaagtatacctttcaactaaaaataccatgctctgcaagaaagattgtcaggggtaaaatataagttcattcagttgatcttgatatttttccttaaaataaatatttctaatacaggaactttaagtctcaaattactgaaatatttttattattaaaatatattttgtaattttatattttcataaaaataaaaaggttatgtgttcatacataaaaatgttaatcagtagagcttattttagcaaagagtaaaaaatactgaggatatctatttttcattaatcagccaagcattatatggagtgcaatataggtgtgctggaaaaaagaggattaaaatttttagatattgctgagaatatgcatgtatgttctttgttttattaattttaataatggaaagggatcagcaattattagatcttaaatgtcttttacctgtaacacccaagagcacagaaggaatcaaagtcaagagatctcagcctggatactgagcaacaccatttgctacctgtaagctggagttcttgatttaaaccctatgtacttcagctctccatctgcagtaggggccattatatctacagtaatcacttcacagggtcattgctaagattatattaaataatggacaattaactactgtgggaattgcaaagcaccatacaaatgctagtcctggttcagtgcctgttctcatatcatttcatcttagctgcaatgaaatcctagtacaaattcatgaccttgtccagctgtcatttatggtataatacataaaagttaacccaagcatagccaaggaaagagacaattggacatacctacatatattttcataactagcaaaaagattcgctggtttttgccaatgtgcttctgttactttttttttcttttactcttgaaattatatttaagtgtagactccacttaaagtaatttgctgctggaataggcataatgttctgatgtctgctttgcttctaattatgttcaagctcattttgacatttaaggccaattacaaaagtctagattcagaagacttttccttagtcatctaaaatttgaacaataaacagtttttttctcctctctctttattcatttaagtggtatgcttggcaacaagcatattttgggaattgtaaaagcaaaataaataatgtgggttcattttgaaaaaagaactacttggtctttcttgccaacataaaaggcagtcacagaattaactgaaaccaccagtttcaactttatttcctcctaaatcacaaggaataaggaattattttacagatacagtgatattttaaacgcctaagttctattaacataaaggtaattgacttcaacagatctctaattccaaaagagttcatacatagagagatatgtataaaaatagctacaaaatctttatcatttaaaagtcactcataattaaactccccagactaggaccctacaccaaaattcagttagtgttccattgcgttttaacatccagtcatgtgatgacttacgacatagagcaaagcacttaatttacctgcctccttattttcagtcaatttgccaactgcaatttttactgttgattcagtagaagaccctgatgagttgacagcaccaaaaggcaattcatagcatctattagtaagaagtcactgtagcttctttgtttaacttttataggtaagatctggcttggaaaaatagactggaaagtatattgtaaaaaaaacaaaattttccaattgactgtcattatcagttaatagattatagaatagtagacctttatagctggaagatatgtatcgtgtacttcaaagatgaaaggaaataatgtctttgaaaacactgtcaaattttcaaaattatgtatctaaaagaaattccttaaaaaatagtttatcctttataatttttgaactttataaaaatggaatcacaccatatatgaccaaacattttaagtcataaagaatacacaggaattctgggccgggcacagtggctcacacctgtaatcccagcactttgggaagctgaggtgggtggatcacctgaggtcaggagttcaagaccagcctggccaacatggtgaaaccccgtctctactaaaaaatacaaaagtcagccaggtgtggtggtaggcacctgtaatcccagctacttaggagactgaggcaggagaatcgtttgaacctggaaggtggaggttgcagtgagccaagatcgtgccattgcactccagcctgggcgacaagagcgaaactctgtctcaaaaaaaagaaaacataggaattccggaatttaaccactgtgatagaagttgcttttccttgggacaatatttttaaggcagatttcaaaattattatttcaactattctttatataatgctttattatttttaaagtattcacatatatgttattttgattttcaaaacaatcttcgagtgacaatgggattgcatatttgagaaaggctggtctcaggacggctgaatgtttttcccaaagccgttaagtaagtcggtgacttagactcagacgctgtgaaagcagaggctgagtacttggttcttgacctcactgatctgctggcttacagagcatcctggtctgcatactcagcgattcaatctctatcttaaacgcagtccctcagccattccttctagaaaggtgatctctttagagtgagagcagtgatggaggtggctgaagttacaccagggggagcagctttgaaaagctcctggctgataggcatgtactaatgggagcaggagctttgttggcagtgtcgaagtccctttggaggctgtgtgaggagggaacccatttccctgggatatagctctctccacagggcagctggatggctaggggcagccatggagtatcagattcacctaggaatcgatatttttcctgagggtttcacaaaaaaagacagagagatggaatttgaagaactgggaagagtgtgattgaattggagggatctgaaataaagaaatagctatggtcaatggctctaaccaacagtactatatttatactccaaattccaaatccacactatgtgtatttaggtacaatttagtttagccatagtaagttgtggcttctggagcttttatttaccataggattttaaatacatttcatgtcatcttcattggttttccttaacccctaattctccacaagatcttcatcccgtcagttatgactttgccaaccaatatactttttagaaatgtaaccccaacattaggtacgaccagcatttactgtatatcaatattatcctaaatattgctgtagctttgaatagctgaattaagttttaaataaaagcaaaggaattataaaacaaaatattaacggattatgaggtcagtgggtttttctgaagataaaactattctactaattattgctgtttgggaaaaatagaaacaaggagactgagtaaatgaatcattaaggcaattaaaaacagaagagaagtcacttttgcactcagtgaataccatcaaggcaagtcttctattttagggctgtcttatagtaagatataacagggactctctcaataaaaaggttgagctactatataaatcatcaagtagattttattttgaaagcaactttttttgtagagaaagagttcttgaaaccagttaaagatttaaaaagttgatacaagttatgttgtaaaatcaaagttatatgtggatgatacgagttgtattccccaccacattttaacatttttagtgtatatatttttaaatgaaaaaatgtacattttacacgtattttagtacaaacagtaaaaaaaaagtcagaagtaatttatcaaaacatatgtttacacaaaaacttgtatataaatgttcataacagcattattcataatagccaaaaggtggaaacaacccaaatatcaactgataaataggtaaaatgtggtatatccattaataaaatattatttggcaacaaaagggaatgaagttccgatgcatactacaacatggatgacccctggccacatattgggtgattccatttatagaaaatatccagagtacaaaaagccatagagacagaaagtagatgtctttgatttccaaaggctgagggagggaggaatggagaatgacagctaacaagtatgtagcttcttttggggataaagagaatgttcttaaattagatagtggtaatggctgcacaaccatgtgaataaactaaaacccacttaatcgtacactttaaaaggtgaactttatggtatgcaaattgtatctcaaaaacctatttcttttaaaagtcaaaaaaaatagtatagaccccttccatagttccttagtcctttccattctagtccttagagttacaggtttattgtatacactttcaagcatttatatgcacacaaaagcatatctatgtgcttgggatatagcaaagggatggtactatatattctacttactctactttgaccaaaacccaagatgtttaaccattctactttttcttaatatttgactatgtttgaacttaaattgatatccacacatactaaaataccaaaatatgcattgagtcatcaaatttcaaaggtcatccatgatattttttaagggcttaaaactccttngcatgatcnttcagcctgtttanttcagcataggaaaatacccttgctttgccttcacctaactctaaggaagtctattagagatatttagggtcagaaatgggttcttggggcttgagagagaaaaggatcttctgctattgatgatctggctctgccatgaccctatggcatgggtcctcagtggccctgtgggaagcctagtgtaggcaacatggcctagcccacctatgttgatgtttaaggggtgctaggaacaaaagagaagctgtggaaacttttgcttatccccctttcttccctctttggtggtcttatttttatccgacgctcttaatctcaacttataaccggaaaatacagcaccaaagcgtgagggcatttaggggaaaaatgttatctgttttggaagtaatatcaaggaagatcagggacaaatgcagaactggaagcaaggactaaggttggatttagcaggatttcctgaaagattggatgtaggatgtgaggaaatgagatgaaccaaagatgtcttcagggtcattggcctgaataacttgaagaatagatttgccattttcttggctgggaaggagtgcagaaagaacaggtttgaaagtggggcatgaggagtttcatatgacacatcaaatacttgagattcctgttatctaacatctgtccaagtaaaaatgtggagttgattctggaggtggggctggaaatataactgtggaagtcatcagaacatacaagggatgtgaaggcattacaatggatgagatcaagcaggtagtgagtattgagatataagaaaagatctgaggtcaagtactggagctctgtaacatttagacattaggaaattgaagagaaaccagcaaaggagcatgcaaagggctggcattggattttgtggctctgtagtgcagtgctctggctcttttggtgactgaaagatgcttgttcactgcttttctctgtgttttcacccggagcatccctgtgacatttctatatatatatatttatttatactctaagttctagggtacatgtgcacaacgtgcaggtttgtacatatgtatacatgtgccatgttagtgtgctgcacccattaactcgtcatttacattagctatatctcctaatgctatccctcccccctccccccaccccacaacaggccccagtgtgtgatgttccccttcctgtgtccaagtgttctcattgttcaattcccacctatgagtgagaacatgcggtgtttggttttttgtccttgcgatagtttgctgagaatgatggtttccagcttcatccatgtccctacaaaggacatgaactcatcattttttatggctgcatagtattccattgtgtatatgtgccacattttcttaatccagtctataattgttggacatttgggttggttccaagtctttgctattgtgaatagtgccacagtaaacatatgtgtgcatgtgtctttatagcagcatgatttatttatttattttttccaactcttttttttattatactttaaattttagggtacatgtgcacaatgtgcaggtttgttacatatgtatatatatgccatgttggtgtgctgtacccattaacttgtcatttacattagctatatctcctaatgctatacctcccacctccccccaccccacaacaggccccagtgtgtgatgttccccttcctgtgtccatgtgttctcattattcagttcccacctatgagtgagaaagtgcagtgtttgacttttgtccttgtgatagtttgctgagaatgatagtttccagcttcatccatgtccctacaagggacatgaactcatcaattttatggctgcatagtattctatggtgtatatgtgccacattttcttaatccagtctatcattgttggacatttgggttggttccaagtctttgctattgtgaatagtgccacaataaacatacatatgcatgtgtctttatagcagaatgatttataatcctgtgagtatatacccagtaatgggatggctgggtcaaatggtaattctagttctagatccctgaggaattgccacactgacttccacaatggttgaactagtttgcagtcccaccaacagtgtaaaagtgttcgtatttctccacatcctctccagcacctgttgtttcctgactttttaatgatcgccattctaactggtgtgagatagtatctcattatggttttgatttgcatttctctgatggccagtgatgatgagcattttttcatgtgtctgttggctgcataaatgtcttcttttgaaaagtgtctgttcaaatcctttgcccactttttgatggggttgtttgttttcttcttataatttgtttgagttcattgtagattctggatattagccctttgtcagatgagtagattgcaaaaattttctcccattctgtaagttgcctgttcactctgatggtagtttcttttgctgtgcagaagctctttagtttaattagatcccatttgtcaattttgggttttgttgccattccttttggtgttttagacatgaagtctttgcccatgcctatgtcctgaatggtattgcctaggttttcttctagggttttcatggttttatgtctgacatttaagtctttattccatcttgaattaatttttgcataagattcctcatacattcctgttgtgctttcaccctctactctctggttctcaccctgttctcatatatccaaggaagatgatacactctaattagaaacaacaactgaactcagctcattattttcaaaatctttcttctccattaggtaaaaatcattgcagtctgtttattcaatatgttttcttttccacttaaattctgttcatgcagagactgattcctgggaacatcaatgtgctggcaactttgactttgtaatttcccttttgttgtcccactcaactgcatggctgatctccattcatttgttcacttccagagcctactgttggcaatctgatttttcttcatagtttcccattaaaattcactagagatgcttttgatttacttatttcttccctggcagggtagagttgcttgttccagatcacctcatagcaaactggaccattctcaagtaaactgctcatccctggtgcagttatcagcagggaggcatctgcagcattaacagtgattgacccgtatagtgatgtttaatatgtgataatgttactagcagcaaacaaaatagcacttgtaaacacagttgtttactcatacttacatgtataggtttggacttgaggggtaaaatagcaaaattacccaactcccctgctaataaaacctaaggaaatgaataaaatgacctgagaaaattataccagcataactacagtaaggaagagctacaatttcatgccattttctccgaatagcagctaggaggaaagaaaagaagagaaagatgaaggaggaggaaaaggaaagggtcgcggaggaggagaagagaatgagagagaacatgaatattctaggctcatcccttaagactgtcttctggaagaagttaagaagaaataaacttggaaaaacaacgaaaccgtatactcaaattccccaatctgtgaagtctgtggccaaagggctatgcagagccctggaaagtgataagaaccactcatgtatagaagccacttgttccagaggcaaaataatccactagatctcaccatttttatttttttcagctgaggaggcttgagagtgaaggcaaaatgtcataccttctgaaaatgaatcatctcccattctgctacaaaaatgtagcataatgaaaatatgaagagacaaaaacagaagcttcaagagtacaggaagctcaggataaagaggggtgtcgaaagccccaggagttgtcctattcgcccatacagagaaccaaaaccaacaggaggacattgacttctccctcacctggagtcagtggagtgggaatgatggctggagagaggccagtgaatttggggaagtgaacacttaggactgattagttctggctgcaataaagatttcctgaacctacctgaattgaagaaatatctgaagcctgatatttcaagcataattgcccatacacagcctcagctaatacttctctccctgtacccctctcctcatactgaaacagtaaaacacagagaagaaaacctcgttgataaattgaaaaaggatgctaatgagtgggatgagatatctggaataattctctgcccctgtattagaaagaatgaaagatctagataataatgttaaaagtggaaattttggcaactgtggaaccagtctagaaagaaattagttcaaattaaaacaggtagtgagaggaccccaagaataatatctatgagagtaagaatcaaatggtttctgggcaattgatggtttaaagaaagaacatctgcaggatagtaagaagggtttattctaaatgataagtgtaatgtgtgagaatctggaagctacttgagatagaagaaagaaagaatatatttanttttcccaatgagacatgtgagaaatagtcagaaaatctcagcaaaaacaaaaccaaaccaaagattgtattaaaaaaaaatggctcaaataacgtgagttaaattggcccagattctgagcaattaatggaaagtaaggaaggttccatttaaccttgaccatttactgtctaatagagtcttggagggggaagtataattttagaacattacctggcccaagagtgagcaatgttttcataggaaacattgactatcaacatattgattatcaacctgctgagaagcacggatgacccaattatgatagaaaaacaaaatgtaaatattgtcaaacttgaggatataaatgtatacatgacacgagatggaaaatgaaagcagagagaagaggtgacctgacaagtggcaatggtaataccttcatctcacactggaaggggtcaagtctttctagtttgatggaaaaagaaatcgagatttacatacaaatttgggagtggttaagggaagtggggagtgatgttgtgcctgaatgaaatcttcacatatcatagcagaagtctatgtctaaagtggataaatcaagaaatcctaaatagggataggcataacgccatcagaagtgaaaacaaaaaattcctgtctcattacttataccaaaataaatttcagatttaaactgatttaaacagccaattcaggttcaacatgtttaaaataataaagcaaggaaagtgtcaaactactgcataaaaccatgggtaaatttatgtctaatcatgcagtgagagaagtctttttaagcaaaataaaaacacttaaaaggaatgacaaattagaccacactgaaatttaaaactgatctttgcaaaaatataagaaatgtataggggaaaatgcactgaacatacatggcagataaaagatgggttttcttatttgtaaagagttggtggaaaccaataaagaaaataaaatggtcaacagggcagaaaaatggaataacatgcgaataggtagttcagagaaacaacttcctctctgccttctttccactgaaactctactcctgtgcattactgaggtcagttgcttcattttcccaaaactgttcttgattcacccaagaggactaaattggctcttctctgttgcccctcagtactttaaccatattttgcttacacaccaaagttactattcttccctttctttttattttcttttttcttttgagacaggatctcgctctgttgtccaggctggagtgcagtggcacaatctcagctcactgaaacctccgcctcccaggttcaagcaattcttcttcctcagcctcccgagtagctgggattacaggtgaccatcaccacaactggctaatttttgtatttttagtagagacggggtttcaccatgttggccaggctggtctggaactcctgacctcaagtgatctgcccgccttggcctcccaaagtgctgggattacaggcgtgagccatcatgcccagccagtattcttcctttttgagaattgatcctgttcatctttttcttcccctctcaattatacaattcttaagggaagaaactatgtcttactccactcaatccccaatgcatcaaacctttagtaaccactaataatccttgtttgaataaataaagagcacaggccctacggacaatgtaagtcagtaagtgcacttgagtcactatctcaagcacactaccatttgaatttaaatttctggataaaaatctccacaaaactgataaggcacccttcaagcaagacacaagttaattttcagcctctaatagaagtatttgttggcactacctttcattggtttattcatttcagcagccataaacacacagatcccattgtcacagaaagccttgttcgcaaatataatttagtgcatctcacaatatgagctttcaggtggttgagctgtgtggaaattcgatttttttttctcccgagactctgaaatgaaaagaggataaccccagtgctcttgcccacatcgcttatctcacaacaacagattacaatcccataactccacgttcccaagcagctactgtgttgatcaaatgtaatgcaacagatgagaaaacatcagctggttcatgcccaagacagctgaaaggtgcacttacatttttagtctccagataccaaaataaatctgttgggcttaatcatgagttgtcctgctacttttggggaaagaaagccatgtccagtgaaatttcttttatactggcaggcattttgactcttcacctattgctgttgaatctccaggatatgtacatgttttcacaccgataccttcattgaagaaaatacagcaatctgatatttttatgtgaatcatctttggaattcgtgttcctcacctttggatacaataaatattcaagagaaaaaaaatacaggaaaaaaataattgccctcaatgatgagttctgaaatacttataaggataagtgatgtgttgaaaaattctcaaaagcaattttagaaggaataatttttgcagagagtggctgtctcttgggcaccataacaagaaaaatagtatattgcccttggtgatggtgcggtcaagtatcaataacaacacgcttcatattttcatgaacaaaacacttcttcataaggtaatgagattatagaaaaatcaactcaaggtaaaaataattactttgcacatggacaaaggataagtggaagaacttatcttctagttcgtggttcatttaacaattaattgctcctttttcaaataggtccactgtgttctgggccttgaacagtgtacctctaagaaagtttgttatccttactgaaggaaattacagttaggtcgcttctcagccttttggctaagatcaagtgtgaaaattatacttagattactatgtaattaggcctggtagatcctgggtagacttcactgaatgcaggatgcaatatttggaaaagtacttactttggagtcctaatgcctgggtgctacttctattagtgccagaaattaatagtgtgactttgggataatcacttaacatctttgagcctcattttccttatcttcaaaaaaaaaaaaaaaagaaggaatcagatgaaatcatctttaccagattaggtctatctttaggtcccccagtgtaactgggtttctgtcttggtctcagtccatccttgcattatcctggactttatttattggaatcccaatatctgagaattcaatttatgtctttatcgtactgaagtctataaagatgtttttaaagagtcactagactccagtggggcaacttgctccagatatcatcatagtctcattaaaaagtatttattgactactaatgtgtataggatacatgagttggagactcacagatacacatatatttaattagaaaaatatgtaagaatgaaactagccaaagatattggcagaaagtgtgtgaaatttttagcttggctggcctttgttggaatgctctattttttatttttttgtttgtttgtttgtttttttggtcttggcttcttccatgatcttgaccctgtgccttgaaactttttttaaatcatgaaaaatgtgtttgtcttcaagctgtccagtgtggcaatatcacttagaaatatttcttttttaaagagtcccatgtttttaatatgatagctaaaataatatgatagaaaggtaccataaaaataaaattatatgcctttatatgccttagacaagtgtaattcactataaaaataagtttctcttagcagcaggaacaaagaaaaagtaatctgttgaaaatgttagatagcttgctgaaaggaaaaaaattaacggcaaaaaccaaagatcttgaatttgtttctaatttgctcaacagcatttactgcgaacaaaactaattttaattcagggtaattattttcttattccacatcaaatagttaaatatcacatttttaagatctttattcttatatctttcctatgtgttgaatcaagactgttcttgccctgatcagataaaaatcaataatattgagatcaaagtcttcaaacatcacatgctaatttgaagttgtatgcaagcctaaagctagtgtgtaatacctttgttcttttcctcttaggcatttatgtccatgttccagatcctcacccaggaaggatgggtggacgtaatggaccaaactctaaatgctgtgggacatatgtgggcacccgtggttgccatctatttcattctctatcatctttttgccactctggtgagttcagcatttttttttcaattatatcttggaatttataatgggatgaacatcaacagatatttggcctattgtcaatttcaaaatgggagccaaaggactaatgcagactaatctggttggatgagatccctccttccattctaagtttctacacatcaaagaactgcttgatttgttaagttagatattttctccatcaggtaccagtgtttgtaagagggaaaacaagacagaacaaaatgaaaatctcacatttacataccaatttaagtatccccaagcattcacacctagatgatctcaggatcaaaccggaactccattattattttttttctcatgtagagacactgtcttcagtttcactgcatcctcttcacagctgtaattaaactgaagacagaagctatagacctcagggtgcagtcagttaattagaaatcagcacagtggttttgtgctatgtgcgactggaaaaaaacacatggaaaaaaataaaacacattgctgcaagtattgtaaaagcacaagaaaactgaataccaaagtccagtaagaaccgacttctgtcacacaagcagatttttctcttcaatatacttccattaaaaacaaagaaaaaatgttatgaaatgcagcctgtgacttactaaggaatgtaataccaaatgctcatctctatgtaataaatttgttcatatttaagaagcaaacctaagttctaacatcacgttagaagttccaagttgaggactcaaatgtttccctttatcaggaatacaagaaataaatacaactgctactattgtttttggcaatatgtcttacagttacaaaatatcttacaatattattgctaatctttggaacacccgattagatagtattcccagttaacagatgataaaactgatactcagaagagtttagtaatttatctgatgtcacatggaatgtaaatatgaccagagtgttcccaggtctgtctgatctagatttggtccttagtcttgcagctaattattattgcttccctggataatgaagtttaaagttgtctgaaaaaatgtctttgtcaaatcaaacttctgccaaagaatcactcacatcaaacaaattacatataatctctaaggtcagttttaattctaaatatctatgaatgaaataagtccttgataaactagatataacttagaagttgtgtttgctatagacagacagaggctttcaaaactaagacatgtccaccaaaaagttaatgagaattcattgttacatggtgtgaagtcatgggctcaaacagtagaactacatagacatagttttggcctttttatacatggagttgacttttaaaagttacgcgaaagataggaacattgtgcaataggaatggaagcctggcttgcaaagctgaatctggctgtgctggtgtttgcacaacacttagccctggggtctttgcatcttcccttccaggacctttaagcctgtgccatttattcattcatccattcatttacacatcatttatgcgttcaactaagtttattaagctacttgtgtttaagttactctaaaactatggaattgctcagactgttgattgtgggttaaatgcaggatagatttcattgaaatcatctatgtcagggaatatcaatctaattattttatagtaatcaacctaatcattttcctaataaagcaacaaaatggtttactttcctcactgaaaatgtttcacactcagatatatgtttgattcttaaatcagggacacaaaaaatgaaagctggaagaaaaaactagcctattacattatttagtaaagtaacattttttcaagtgaaaattctactggggtcactaatataataaaataaaatattaaaactggtatatagccttcttgggcaatctaaatcttctttatgggctaaaatatggcattggttatagaacacttaaagttcatccaaggactcctggggttcacctgaggctttaggaatccacagagcacaagatgaaaaccaatctctaattctactctttccacttactgatttaagtatctacccaaaacatctgttctaacttttttattgcaattttcaaacaacagttataagttaaaactgaaaattgaaataacttgttcttttattcacactttttaaaagtctgtaaggcaattgagaattcagaagtctacatgttaacgagaacagtttggtaaatgtggtgtcgtacaagactacgaaattggagggcaaaaatccagattcaggttctagctctgctggtcactcgatatctgatttctgaagactcacataacttccctgatctctctgttgtttgtatcggaaagtgaggataataattcacaatacacccagtttccaaggttgtcatgtatctgcgagttaaccctgtatttctgaggatgttttagagcaccagcactgagcccctttaaatgtagataagacggtttgtgcatttaaagccatatattaataaatgttaaatttatattatgttcagatatttttaatttatgcaagtatcatttattagggtatcatttatgccccttcctgaattaatttccattcttcttctttctctccttccctatacctcttctcccaggcaatgactctgataattttaatataaattcattttccaatttttagaacatatttcactaaatttaacattctgtcattcagtagaagactcagtttgtgtaacactaaggagaaaaacaattaaactatacacatagaggataaggtgcatccctacttcagagtttttaaaaggtgaaagtaaatgtgcatcttaaaatcaatgaaatgcagaggtttcccacacatatatgcatccattaaaaatatagtatttagggccagcacggtggctcacgcctgtaatcccagcactttgggaggctgaggcaggtggatcacctgaggtcaggagttaaagatcagcctggccaacatggtgaaaccctatctctactaaaaatacaaaatattagccaggcatggtggtgagcacctgtaagcccagctactcaggatactgaggcaggnnagaatcacttgaatctgggaggtgggggttgcagtgagccgagatcatgctgttgtaccccagcctgggcaacaacagtgaaactccatctcaaaaaaantatatatatatacacacacacagacacacacacacacnnnngtgtgtgtgtatatatgtgtatatatgtatatatgtgtgtatatatgtatatatatgtgtgtgtatatatgtatatatatgtgtgtgtatatatatgtatacatatagtattttaaacttaaggttaccatctaaatacattttatgcccaaaattctgaagattaatattaagttttcttagtttgcaaagcactgctccagtcaactgatggccatccctaggggagtggtctggtaatacatacttctctcccntataagtccacgctggggccaagttatacatggtcatgtgtttccctccttatcaattatttgtagcagcaatttttagaacaaatggtatcaaactcaacatatattctgcagttaaatttttcactggctatttttttctgagatctttttatcttactccacatgaagctaatttggtcattgttatagagagccgtgtggtactccgcggactgtgaatatgccatatttgtttatcagttccctttctgatggctatttaggctgttttccaaagttgtacttttataatgctgcaatacatttatgtatcgtgcccacttatgtgaatttctctaggatatatgtccaaaagtagaatgcttagatcttaggtacttcccaaattgctctataccagggacctgcaagcccagataccaaatccagcccaccacctgtattatacaacctgtgagctaaaaatggtttctataatttagtggttatacaggtacccacagaatatcctcagtattgcctcatgaccctcaacgtctaaaatgtttactgtctggacctttaagaaaacactgtggactcctgctctatagataaattgtgctaatttaccctcccaagtgcaatagttgagaattcctgtgttcttacactctcacttagacgtcacattgtttgacatgtgaggttttggcaatcttatgagtgaacactggtatcaattgggcttgcatttttgcatttccataattattaataagggagcacctcttactatgtttattgaccatcttttttgtgactcaattgtttgctttacctattttcctaaaaacaaatttgtcttaactcatttattggatatttgttggatattaatctcttgactattacatgtgttgcgaatatcctctcttaggcagtattttgtcttccagtttgtctgtgatgtcttttgacattctatatatttttatttcatttttagttaacaaataataattgtatatatagtagtcctcctttgtccccaagggaaactttccaagattcccagtagatacctgaaaccatggatagtacagaaccctatatatactatgtgttttattatacatacataactttgataaagtttaatttgtaaatgaggcatagtaagaaattaacaacaataataacaaaatagaacagttatgacaatatactgtaataaaagttatgtgaatacgcattctctctctgtctccatctctccttttcaaaatgtcttaatattttcaggctgtggctgaccacagggaactgaaactacagaaagtgagacagtggataaggggggggcctactgtatttatggggtacaacgtgatgttttgatacatgcatacattgtggaatgatcaaattaggtcaaaactatcggtttttcactgatggtctgtatttattttgacctcatgcttaaggaattctttcccacctgaggtaatacaaatattctcctatatcttcttctaattgtttacaatttttgcttttcacctttaagtctttagctcatctctacttactagttagatatagcataagctagatgaaatatactgtttaaataaaactcaaatcaatttttctcagatttatatgagaaactgcaaggtttagaatatttatttgatttatctgattatgaagtctaacacaaaaaaggtaaactgttcaaacttttaaatttctaaattattttttaaatgatatataatttaataaaaggaaacactatataactaaattaaaacaaagattatcacttctatactacccaaaaaaagtctaatactgaagtcacttccccagttcacaaaaaattttgcttaaaacatggtcagcagttgtttctcttttcctttgtacccttagactaacagttactgtaatgggctttatgaagtgagttcaatttgatttgatacagttttactgaacctctactaccacgtgcaggtactaacctacgtcctggccaaagtgggaaagagcttaattcatgtcctcttaggggctacaaataattggaaattagcaataacgaacacagacaggctaaccaacaaacaaataaaaccagtggttccaaggcctaaagatgcaaatggtaagagaggcatgtgtacattgctacttgctatgtgatcaataaacatctgcttcccagttgttcccaatctgccttatgagaaatatcaaaagtagtggcagtgaaacagctggagatagtctgtgggctaacatataaacacatgcgtgagggtgacaggtctgtgttacacacacattgcatgcccctaaaaactaaacttcacaagttaaaatattccataagcctgtcttcagagctgaacttaccttaaaatgagcatttttgtttaattgtatgtttccttttacaaaatgctacaacgcattttaacccaaatgggtgagaagggaaaaaaaaagtagtctgtgtaaacatacaagctctgatgtgtcttttgttcacaccgaacagtggtgccatacaacttgatgagttcctgatttagtcttcccagggacaggcatgtgccaagcagcgaaaaataaaatgtgtggttgcagcttgttctggaataaagaacagcaggctatttcatcacccctgggggcaccaattttcagcccacaatgaaaatcaagctgaagtatatagcaaatgcatattcataacctccatgtattgtccctttcaaacatctctctcttccgccaggtatgtattctgtatttctgtcctgactgattgcagtttattgaatgcttgttaaattcatctttttccccatctttgccaatgatttggggatagggactatggaagacctaacaagttgaaagactccatttttcctgcaatgtttttctgtctattactgtttcaaaggaaagccctgtgatgtgaatactgaaaagaaagaaaatgtaattaagcgttcattgtgggtagtctttttgttacctttgaagtgctgaaattaacacagcaatctcttgattttgcctcggtagtggcagcttgctgagaatagtcatattttgctgacacaaatgggcattccaaagtgtaaattaaataacttgattgtatagaattcatttaaaagtgcatctgtgttgaacgtatcccattttacactgcgctgattacatttgactttttgttgacccatataaaaagtgaaattgtttcataccacaggcggcaagacagcttaggatagggtgattataaatggtaaacaggaaaagaaaaacacaagagtgtgggacattgattcctgctgtgtttctagtgtatatttcaaaataatttaaagataagaaattattaataaagttcacagcaaagacgaaaataagattaaaggtgaaatttcagctaaagggacatctttaagctatataaaaggaaaaaaatcataaaattaaacttcataaaaagaaagaaaaatgaaaagggaaagctatatagggtaaggtcatggctatctgcagagtcagagagaatgtaaaataggatatgttgctgcagggagagaaattttcaaccagaaaatccattgtgaatgacaaggctgccctcttgcaaaaataaatcatacttttcagctgtttgaatcatgctaatccaaaagtcttattccgagggaatgagaactacattatgttggacaaaatgatctgcatcacaacaaaattggaagaaataattatacttgcaaatgcaactatttcagtaatttattgaatttgactgaaatttattgtagttaaatagaaaaaaaataaatatatctgtggtcacatgtataaaacttaaacagagcaaataactaccacagncattaactgaggtgaatctgggctgtttcagaggnagaaaaaggaagaggaggtattagagaaagaggagcgtcataaagaatattagagaaatcaaacgcctgtgagaccctggtccaggtaccagtttgaacctggaaaattacgcttttgaaatccactaagctgaaaggtcaaaatcctattatatcattcataaatattctgagagagacttcccagacttcagcaggcctgataatgtggcattttccacactgaatgcattgtcaatgctaaacataccttagcaatgggcattataatctgagggaaataaagaattagatatctgaaatacattgttattctccactttttcattgacatcactgattcataatgtatcaatatcggtcataaaattaaaaagtggatacatacatcagctgtatattacacagtaggacaaatgctgtgttgcatattaatatgatcaaacgctatagacctgttaaaagtgccaaatagtaaatgcatgaaaaaatagatatttggctttcttttttacagcattgtacaaactaaacaccactgtgttgaaagcatacatgtacttagcagggttcaagaggagatgaaaggtggtgtcattagcctttttcccctgaaattatatcattaaaaaaaaatagggacatagtatatcctgctgcagtataaattgaaatatcgggccaggcgtggtggctccccctataatcccagcactttgggaggccgaggcgggtggatcacctgaggtcgggaattccagaccagcctgatcaacatggtaaaaccctgtgtctactaaacacacaaaaattagcctggtgtggtggcaggcacctaaaatcccagctacttcggaggctgaagcaggagaaccacttgaacccgggaggtagaggttgcagtgagctgagatcgcaccactgcactccagcctgggggacagagagagactctgcctcaatcaatcaatcaatcactcaatagaaatatcacataaatctaaatattaagaaataaataatgcaaagcaaattagttgcataatttagaacagtcaaagcacaaaggtaaaagtgttttatacatatgatgacactcctgtgatttaatgtggcaagtccctgtaaaattcctttattacatcaataccgttttaatatgaacctgtaaatcacatttagtggctatagaaaaaccttttggcacagcaacatgtggtagtgtaggtaaacattagctgaaggtaatatgcagacatcgctgtaatgggttctgattactaatgagcattacctcagctttctgtaaaaacagaataaatgagaacactcagagaactgaaaaatggttcttcctttcagaatgagggcttgtcacccactcccatgactttccataccatgtgttggcaagtgtctgacacccaggtcgacctgctctttcagtccaaatccacattgcactgggtttgtaggaaaatactgtatcattgataacataaattaaatatgatggacaccaagctgccattcttccttcacttctaccctttatcgcagtgcttattgtcaccttattagagtctgtgagaaaagaaccgcaaagtcattctcacctcctggcaatcagctacagactcaccctgcacctcctgaatcttctgtttccattgcaaagacacagccaaagccttgggtcagacttcagtcatctctcacccggagtgccagttgccaccttatcacagtgccctacatgccagggacagtttgatgaggatcaatctgaaagtccacattcaaatagggcttcagcggaaacccaagccccctaccacacacatacatcttgaaccagtttctgttttgtaagtttgtgcttttgtcatgccactgtcaaattaattgaaaaatgatgcctaaacaaagatatttgctgaataaaattaggtttatgctactttcaagcaaacttcaaactttattatattatttttaaatttgttgcctccccaagagtttgttctaattctttaagtttgggccttcctgttgtgaagtcacgcagcgttttcccagagaagccaaagctcttcctttaggagtcttgccttctcctatccacagcaaatatcaaatgcatctgccttgaggagtcagtgcagctggcatgccgtggctctctctgtcctgctctgagggtggcttttctctgtttatcccaaggaaaaagtatataccagtttcttgtttaaccaatgtaagaaaacattacatatcttttttttttttttttttttttttgagatggagtctcgctctgttgcccaggctggagtgcagtggcacgatctcggctcactgcaagctccgcctcccgggttcacgccattctcctgcctcagcctcctgagtaactgggactacaggcacccgccaccacgcccggctaatttttttgtacttttagtagaaacggggtttcaccatgttagccaggatggtctcgatctcctgacctcgtgatctgccggccttggcctcccaaagtgctgggattacaggcgtgagccactgcacccggctagaaaacattatatatctaataacattttgtcaaactgcacttaaccttgtctagttactatttcagtgagccagtttatttttttgttttgtttgtttttagtttttggagacagggacttgctctgccatccaggcctagagtgcagtggcacattcacagctcactgcagcctcgacctcccaggctcaagaaatcctcccacctcagcctcctcccaagtagctggggccacaggcatgcgccaccatgcctgactaatttttgtttttttttttttttgagagacagggttgcccaggctggtctcaaattcctggggtcaagcaatcctgcctcagcttcccaaactgctaggattacacgcatgagtcaccatgcccagctgagccagtttaaacttgctgcattacatgcattaaaaacaaaaacacacacacaaaaagccccagaacttttaaaaggaaaaatttagcagaaagaaactgatgtcatcactttgtgattattccagcctccctctcttatccgtgacctctgtggcactcaagacgcctgtccatcctgccttcctctccagcctccaaaccctctgcctggaagccagactctggccagacttactcaccaggccctcaccacaccacctggaatgcgttcctcacagctcttttccaaaatcagtcttctccttggcatctgcgtggcttgggagagcatccttggctctctgtcattgtctcacaatcttcgataagtagtgttcggaaagtgcccccctggcccatgtgcccagatgggctctccaagtctaaatgcttcaaaatggtacagaactaaatgtgtgaatttatgatggggcttcatttctggtcctccttcttcacacttgcgtggccgcccttcccatccgctctccaattttctttcacgtttctgctttctgagaagaaataagactgatttctgggatggagcatgagagagacagatgcagagagagagagagacaaagcgtgtttccgtgggagatgcccacacacctcacttgggagcatcggcagtgttggtacccacacaggtgtgttttgacatccaagacttgaccctaattcttcttggaacctgatggggagagaaccttgctgggggccccacatgggccaggggcagccagggcagagttgggcagctgcctgagagttctcggtggagaggcccagaatatcccacaagaacaagtctccgttagttccaagtggaatgacagccctttctgagtttccctgagttgagagctaatacatgtgttgtgggaacaacagcccaagccaaaggccacgcggaggccaggcgtggtgactcacacctgtaatcctagcactttgggaggccaagatccacggatctcatgagctcaggagttcaagaccagcctggtcaaccttgtctctacaaaaaatacaaaaaaaaaaaaaagtagccaggcatggtggtaggtgcctgtagtcccagctacttgggaggctgaggtgggaggatcgcttgagcccatgaggtggaggctgcatgagttgagatcacaccactgcactccagcctgggtgacaatgtgaggccctgtctcaaaaaaaaaaaaaaaaaaaagaccaggtggagagtatagtggattaaatgggcccctacaaaagataggaccatggcttaacccacagaacctatgaacacgaccttattggaaaaagagtctttgcagatgtaattaaattaaggatcttgtgatgagatcatcctggattatctgggtgggccctaagtccaatgacaagcgtccttgtaagacacaaaagagcacaagacacagagagaaagccttgtgactggtgttagataaaggccaagtgactggtgttaacaaagccacaagccgagggacagaggaggccaccagcagctggaagagacaagggagtattctcccccgtggcctttggagagagtgtggtcttgccgacaactcaattttggactctggcctccagaattgtgatcaaataaatgtctgttgtttgaagctaccaactccgtgcacggtgttacagtactctaggaagcgcatatggaggacatcactgggaatcagagctcttagctttgggagctgagatggtggtcaagtcagcagaaagaaagccccaaaccccctgtacagcctcatcagttcccctgatgggggatggaggacatattacccagcttgtagtgggtaaccttgaccttaaactaccctaaaaagaaaaggagcatttattcaaatgtaactgagcttgaaaggactgaatggaactgatgtagcaagataatgtttccagcccccaacagaaatggagggattgtgcaccaaattcaattcatttgtagaaaaaccaagaaatttattttagctttgcataactaattttatgtctgtatattttcagacattgtatagctatactacatgtcagggttgctcggggagaattaaatgaaaaaaaagctgcaatttgttgagctattattacatttgtaatattttacaaacgagagcaccaaaactcactgtgggctggcagttgccacgtggctcatagctggcacagcctgtttgctacctgagctctctgactccaagcacagtgctctcttctatgtctgcttaattcagtgataaacatcactgcagtgagtgcaatattgtatttatgtatcagaccaggcagtgcctcacacatccatggagtccagtcaactgtttgtcaaatttaatagaataacatagccttgctgtccttaagtagcctgccctctgtccagtttgggggatataattaagaataaaatctcctgccaacccacgaaacctctccttaaaggtagaggagagagagcccagttttattattgaaaaagctctaacgcaggctgcaatgcacatcactggcaatctgctaaagagatcacggaggcagaaaatacctccctcttttatatcaccgctttccatacgtgttctcaagatagaaataactagtcctcaagtaagaaggcttgacagcaccacttgttaaatatagtttatcctaaattcacctggtaagtggggtggccatctgtgggagctgattgcctttatccaaaggaaaattaaaacttctcatatttccgtgacaagtaggtaggtttacaacttggagccagatgtctaaactcccaccctctacggagaaggggagacggaggctcttgtcttccttgatgtttacattacttcaaggagacagccctcaaggaaaacattcttttaggttgtagaactggtgagaagcttatttggctcttaaaagtatttatatacatctcaaacagaaagggaaagaatttacaattccaagtcttataaaggaaatgctctaagaaaagggaggcgaacaggatcccttccgacccccctttttttagattcatgtttgtactttcactagagagagaggaaacatgattaaattaggacacagaacaatgcctgctccagtgcccaggtgtgagagaaggggtataagatatcccctcctgcagaaggaaaccagcaggtgaacatctcagaattcggggtgagaaatcaagacaggggaaggtagactctgcctctgatattactttctgtcgtcgaggtattgggaaatgggctgaacatggagcaaactttaaaatgtccagtttggaatgatgtctggttcagaataagcctgatgcccagttaccagtggtgtaccagtcatggtgatgttaataaaaaccctctggtctagatcattctgtatcttcttcaagttatggtaagactccaaagggcatggctgttgaaaagaagggtgctggcatttgtcccaagccaagcatatcccatcataatttaatcatgttgatatactcattaaaattgaaaaagagctagccattaacttctgcataataacttctctagcactggctgaattatctcaactttctgaccttcatgcttaaccacctaatattaacaaatgggaataattcattaagaagaaaactgaaaagaaaaatatgagttagaaatgaaaaagatgatcaagtgaatatgggacttaaaaaaattcaaagttgaagagggactacagcgtaaaatatttttaaaaattatactattttgttttattttatttacctgttctaaaagaagcatacattttttaaaaactgagaaaatcataacattttttaaaagaggaaaaattgtattactccaatacacaaaagcaagtaccattaatatttctaaagtacctccttcaagcattttctctattactccgttatatgcatagttgagcttctaccatatatatatatatatatatatactcgtatttactgttataatgtgttttcaacaccataaaaattcaccagtgtattaggtatctgttgctgtgtaataaattacccacacattagaagcctaccaggaaatagatgtttattatctcaattagtttctcaaggttgggaatctgagacaggcttagctgagtgtttctagttcaaagtgtcatgaggttgcagttgagatgttgtctacagctgcagtcaccttgaagttttactagggctggagtgcctctttccaagatgggtcactccagtggctgttggcaggaggcctcagttcctcaccacatggacctctccagagggctgcttgagcatcctcacaacatagcacctgcttccacagagcaggtgatctaggagagaccaagggtaaagacacagtgtattttattgcctagcctcataagtcacacacaattatttctgcaacctgctgatgacacaggccagacatattcagtgtggaagggaactacacaaaggcatgaaccaggagtctagaacaacgctggaggccatcttgaagagtagctaccacatgggaatttgtaatggctgcatgtatcttatcacataaatctctataactgtattgactattcatgcaaagttggatgttatttctaatatttccttattacaaacaaccctctaataagcacctgtgtatattaagcttttcatcaatagttcagatcacttttttagaatattatcacacaagtgggattgttgagttcaaaaaaacctttttcttaatttcctttttaagtaagttggtttcataaagcaagttaacatattatttcaatgaacgtatgcattttatttcatgtatttgtgataaataccttgaaatgcctatgcaatgattcatcactgcatgtttattttgcatttaatgaattatatactcaatatattttaggtagctatattatacatataatattttgatacttatggcatgtgggttttttcattgttatttcaccattttatagatcctcctgagtttgtttgttgctgttattttggacaacttagaacttgatgaagacctaaagaagcttaaacaagtaagtagtgtgtgggggattttttttaaaaaggacaaaaaaccaggggcaaaccatttgcttataggcaatattaaaaatttgagcttcaaagtaaaaataaaatgtaataaggctatcgaaggtggtaaaatggtgttaagtttaacttttatctaatattaacaagggaaactttctattactctataaatatcctaggaatagttttattatttttgaataaaagtaaaatgggcagagtatagttaattggatttggagactgttctccttctgccattaaatgaaggttgtttggaaatgagatgcctcaaggccagattttgcataattatatgtaatagtctttatcatcatcataaaaatactacaatcaacctgcctggtctctagacactaaaggacaatttagtctgggtgcccatagtgagtataagaaacagaataaatgatcaaatgtggacccagttatgtatgaaaacagacgggatgtcatcatggtgaaagttataccccagagaatgaaactagaaataggtgaatgctgtgttcttaatggcaggaggattacagctatcgtcaatcattcctatagcttctgttgcatggaggatatttggcttgaatatttggctctatcaaagagtacagaccctttgagaagaagagctcgcaagagagtgggacaatgggtggaacgttcttgtatcaccctccccaaaactgagagaatagttgagatgttaactaccaaactctacccaactgttagaatgttataatgttgacagaaccacacctgttataatgttaacagaaccatggctctgaaactttccatttggaattataagcaggccctccatattcagaatttcccaaagctctctatttttaacaaagcaaaaagtataacctatttaaattttttaattttttttaatctatctcatgatcaggttaggcatttcattttattactcttacctttaaagcctatgcttttaacagagggtatcaacaaggaaaaaaaaaaacagaacaaacaaacaaaaaaaaattgttatgaagattcctacttggagcataccaagaaaggttttgagttctttaccatcctacagctacccaggcaaatgggcatcttcccggctctgctagagatgatttcttccccgtcttttcactctgtggcagcagtgatagattcttcaaatgagagttgggacattcctttatgccatcaatgaaaatactttgtgtggcttctagttgcagaaaaaaatatctaataggtgaattcaagattgaaagtgaccagccatataattagacatgatcaataaaaatctgctgctgtagaaccactttagtccatgctttacaaatctggtattgagctgtcacttttccttcatgccatggttattatgcacagatatgtactatcctctgagacagtatgttacaatgtgtattctatagataaaatttaaaatatccaattttttttagctagtgctaccgccaggttccatatgcttttaaataaccatataattccagccctcatttgtaaaataatttttctgctggaaagcaacaaaataaacttgtatgtagcaattttttcaggattgtgaattcaaacctaaggatactttgcagtgttcatttttttcaacattttaacttatttatatatacacgtatatgcatagcctacttaaattttttgtcatctttatatctagtggtcacaataggcatttcatatctgacccaaagtgaagaagcaacaataaaaagtcaggactttaattggcattgacattcttcctgggctataaagttttaataaactatatggcccttcactttgggtaaatgtcattatttataaaatggcttttaatcacttgctagatattagtgaagctttgtgtttctggaaattctgtatatcagaactggttatccaattgcactagcctacaatttggaccatatactctgggtatttgggcattgaaaactccaatcataacgcagcaattccatttttgttagtactttttacactattctttattcatggttggactgaaactatgtatctattaaattcttctattaatttcatacttgaaactcgttgaacctgaaattcattatagcaaatattattaaactaagttaaagtgttcctggttaactttctctcatttttgtatgattatgttttgttttcagtagtttttaacaacggtgtcattttttatgcccctgatttttcttattttcagtgatcttggcctttaaggtagggctcatttgcatatttttaaaaacacacacattctcatgcattcactttcatatacacatgtggtcatgtggatacctgaaactttagactgtcttttacagattcttttaaaaaacaatgaaattggtaccctaaatatttgtttcaattgtccatgtacaaatccagtccttaagatttaaacttatgtactctatttttactactcccagatagagagtagagtggaggaaatgttcaaaaaaagtatttgattttcttaatgtttcaagtttcagaaagcgctatgcattccaaattccatctttgctccgttcaaactttttgcttcttttaccacatctgcttgtatgtagattctcatagctatggtgacattggttgaattcttgctcagtatcaagtgttttatgttttcacttatttttcacaacgacccattcaatgcaggtgttattattatgtccatcttacagatgaggaaactaagacatagagaggttaagtaacttgctcaaggttacacagccaacaaatgtcggagctagaatttgaacctaggcaatttggaccgaaagcccacactcttaacctctaaactgtattgattcaaactaaagctatataaaggccaaacatgagtcatgcatactggggagggcactcataggactatgaattatttgtttataaataatgatgttgtagataacaaaggcatcctcatattcaagtaactgttagaatgaaggacagtttctgagaatagcggtgaatgcagcaacccaatcgttaaaactattcacattagttctttgtttcctctcaaaactcagaggatctaatcagagccactggagttacagtcttgctgcgcgatggcaaacgccactcaaggtgccactttgcanatgatcttcttggagtgatgatcctcctcagacgcctacataacaggcttgcagcatagcagcattttagtgactttaatgtcttttattatcgatatgtttttaactgtgttaatatgtaaaagttggttcatagatttttttcaagagtaataaaaaccagggcctgttcgctctaaaatgggggtgttggccaggcgcggtggctcacacctgtaatcccagcacttggggaggctgaggtgggcggatcacgaggtcaggagatcgagaccatcctggctaacacggtgaaatcccgtctctactaaaaatacgaaaaacaaaaaaattagctgcgcgtggtggcgggcacctgtagtcccagctacttgggaggctgaggcaggagaatggcgtaaacccgggaggcggagcttgcagtgagccaagatggcgccactgtactccagcctgggtgacagagcgagactccgtctcaaataataataacaataaatgggggtgttaacatgcaacactcagtagggctcaacagaggaagaggaagcagccctcatacaggggagaggagaggccgcccttaagctaagggtgtgagatggtggtagaaagtgaaaaggtactagtgaagaaattataatggaaaagaaggaagaagtgaagtaggaaaacaccagatatgaaataaagagtatgactataactcagggataaaggtgcaggaatgtggccaaatccagagttaggaatgtgagcaagtcagtggccaaaagtcaaagcccaccagggcgttcccctcagaagctccctgagggcaggcatgggctgcatgatggattgtcggtgggcccgtgttctcctggaagagaagcatggcgctgtatgtcttttcccattatttttctatgcaacctccgtgtgcatcagaggtggtttctagccaaggactgctgagctctaggcgtcctcatgagaagctatggtgtcatttttaatacttcaggaagctggaagagaattgtgcattcacttaatataagcccgcacctgctaaataaaatgtcaagtttaatacttttagctggaaacctgaaaattctaagtggcagcactgtttccttaatactatagttctgaagtgttttcatatatagtttcctttaagcaaatttgtttaaagctacaatgaatttcttattgcttaatcgaggcattttggcaggaaagaagtttgggttttagaatccagaggagaaagtaaaaggactttttgcacagaaattttgagaggctctggtgtgtttattcccatcaccgcctctccaagacatcaccagtgtgtgccgtgatggtcacctctatggcnttttcctttgtcacttcccacaatcatgttccaaaaactcacctcaatcatataaccatatgaaattcctctattgccttccctgtgtgttatatatatgacacagctgattgctaatagcaatagggcagcgtaatcttttcatagctgccatcatttacatgacccccatattgaagtacgtagaaaccatatttctcttttcagcatccccaaaagccaatacagagacctcggaaaaaaatatatatgtgtgtgtgtgtatatctattattacatatatgtaatatatgtaacatatatgtatatgtcatacattaaatacaaaaatatattataattttgtattacatatatgtaatatctatacatatatgtatatattaaatataaagattatatttataaatatatgtaatacatgacataaattatacataatgtgtgtaatatatgtaacagtatataaaaattatattttttatatttagtatttatacacacatatatataaaacattgtttctgcatgacatttaaaaattggctaaaaatgagattgtggtgagctgaggagattcctgataaatcagttggtatagaggctacaacaaaacccttgacctaaaacttctctgaacagaagagttgatgactgtgaagctcaagtgatgcaggaatgacactggggaatgtacacagagttttgcaaggtttgaatatgaagagaattcttgcagctttaatgatttttgcaaatttggtgaaagacagatataaagggttttcttttaagtggtataaaattcattaagctttgagggaagaaaaaacccacagaaaagcctgaggggaggaatcatcgaaaagggaagtataggtagtctgtttagattaaattcagagctgtgaaattgaaaactgtttacccagccctgtatccagtctcacttgaatcctggttccaatttaatgatgtttggtctttgctagagtccactgcatagtgttggatagatattatatattaagaagctttcctttcttattgactgtgacttttttaaacaaaaaccataaatatcttatattctcttttgttaaagtatgagctctaggtgaatgtttttctttagccttaagctgcatgatagttgattttatgtgtcaacttgactgggctaagggatgcccagatagctggtaaaacattgtttctgggtgcatctgtcgggtggttctggaaagatttgcatttgaattggtagcctgagtaaagcagacctccttccccaatgtcgttaaaccacttccaaagctttgaggtcccagatagaacaaaaaggtggaggaaagctgaatttgctgtctgtctcattgcctgcaccatgtaaacaaccttctcctgccctgaggaatcctggttcttaggccttcaaaatgttgccctggcttccaaaggtctccagcttgaagagagcagatagtgggacttatcatcagcctccataattacatgagccaataccttaaaataaatactgatgtatattttattggtcctgtttctctggagaatcttgcctaacacacctgaagctctcattkgcatgtccattttgtcgcctatattgagttcattccagattgagggcggtagcaaggtggcagggatactgctggaagatgccattcggctgagcactggttgcatctcctgatgttagttgaatcttcaatcttctcagcattttgtcagtctctcatgaatgaactcaaactaatgtaattttaaaagactgatataactgctgcaggagcaaatgctatctattgagaactaccttgtgtaggctrctttatattaaattatcacatttaatcctaaatattaaagacgtggcaactaacattcaggggagtgaatttctcacccaagtccacacaattcatatgtgcctgggtcagagccacaaaggctgtctgaccctaaaccaatgtccttgctgaaaaaggccatgctgcctgcagagaaccagctccttcatttataatgaaaagcatatctaggtgtggattggatgagatgcttatccattttttagatcagcatattttctccctgtgggtattcaaagaagaagccaaattataatcagactatagcttagtgagccaggaatcaggatggagagggaggcgagaaaggttgtgtgatttaaatacatagatgatggtttgatagatgcagcaaaccaccatggcacatgtaagcctgtgtaacaaacctgcacattctgcacatgtatcccagtatttaaagtaaaatctaaaaataaataaaaatatttttaaaaatacataatggcataggctaagataggctgagatcaactgagttatctcttttacagttgagatattagtgacctagaaactaaatctactcattctgtgaaactggggtctggacactcatctactatgctatagatattgtgttggggctagggacatgtgatggtcaggacactgtttctaccctcaaaaataactcctgagccagtgggagaaaaagacaagtaaacaggcagtttgggttcagtggtgatgattataaggataatgtttcggaagcacacagaaaacatctaaagcagactgtgaaatcagggacgttttctcacaagcaggtgcagactgaaatctggaggttgagtagtgctaatccaggtgaagagggttggaggaagatggcatgaaggagggtagtgaagaccaagggaactgcagagtcttttttaggtgggaaaaaaacagagtggctttctcgacactaagagtttgctgtggctaatgtgtagagcaagagagaaaattgttaactggaaaaataaggagataccagcacaggaaatgcttgagcagcactaagcattttgatattattctgatggtattgatgccacagggtgtcataatcatacctacattttggtgtcagtgtgaaatgcactggagggcacccagactggagatggaagaatccgttcttgaggcaagtggtgatggcagcctgaaccaaaggaaataggaatgagagaatatcgaaagcggtggagtacagagaagaggatttagtgacaaattggatgtaggaagtgagaaaaaccaggacacagggatgtcacccagatttctagatttctttaagtgcgctgatgcagaagaaatacaagagaaagaagaaatgagagttttgtttatttgggttttttttcgggggaagaagtttggtcatattttccctgagatgcctgggaaatatctatgagacaatgtctatggagagatacaaagtttaaagcttaggtggaatatctaggctgaaatagatactgaaaaagagcaaaatcaacatgcagatggtaatttaagcctttagagagcaggaacttaccaagggaaggatggttccctaaggggtaataaataaatggtatgaaccagtgaggaggagccaaaatgtgtatgagaagtcacagcacaagaggtaagaggaaagcctagaatgtgtttgtcaccaagatcaaaaggtgaaagagtttcaagagagatccttgttcaacgatctcaaagacttcagagaggtcaaaaaaatagtcaccatggaaagcaatcccatttacagtagcaccaaaaagaataaaatacttggaataaacttagccaaggaggcaaagacacataccctgaaaactataaaatactgaggaaagaacttaagccacaaataaagatgaattctgacttacgatgattcgacttaacagtttttgtttttgtttttttttgacagagtctcaaccttttccttaggctggagtgcagtagcatgatctttggctcactgcaacctctgcctcccaggttcaagtgattctcctgcctcagtctaccaagtagctgggattacaggcatgcaccaccaaatccaactaatttttgtattttcagtagagacagggtttcaccatgttggccagggctggtctcgaactcctgacctcaagtgactcacctgcctcagcctcccaaagtgctgggattacaggcgtgaaccacctaacctggcaacttacaattttctgattttatgatggtgcaaaacagatacacgctcagtatgctcctcaacttatgatggaattatatccagaaacactcatagtaacttgaaaatatcataagttaaaaaatgcacttttacagtattttcaacttataatgaatttataaaccctattgtaagttgaggaacatctgcaagtgaaaagacattctgtgttgaagaactggaaggcttaatattgttaaaatgttcataccatccaaagcaatctacagattcaatgcaatctttcgtaaaattccaatgacgctttttgcagatatagaaaagaaatcttaaaattcatacgaaatcttaagggataccaaatagccaaaacaattctgaaaaagaacaaacttgaagatgtcatacttcctgatttcaaaacatattacaaagataaaataatcaaaacagtgtgatactggcataaaggcacacatatagaccagtggaacagaatagagaggccacaagtaaaccctcacatatatagtcacatgatcttcaacagtggtgtcaagaccacactatggggaaaggacagtctcttcaacaaatgatgttgaaaaattggataccacacacaaaagaatgaagtttggcccttaccttatatcatatataaaaattagctcaaaatggattaaggaccttaacaaaagacctaaaaccataaagctcctagaagaaaacaaggcaaaagcttcatgacattggaattgtcaatgatttattggatatggcacagacagcaaaagcaaaaataggagactgcattaaacttaaaaacacttgcacgtcaaaggaaacaatcaatagagtgaaaagccaacctatgggattgtagaaaatatttgcaaatcgtgtgtctgatgaggagttaatattgagaatatgaaagagctcctatagtcaacaagaaaaatacaaataatctgttttaaaaataggcaaaatacttaagctgacatttatctaaaaaaggtatacaaatggccaagtagatgaaaagatgttcaacatcactaatcattagagaaatgcaaatagaaaccacaatgagataccactttatacctattagaatggccactatcaaaaaaaaaancagtttggcaagaatgtgaaaaaattgcaacatttgtactttgttgatgagaatgtaaaatggtgctgctgctgcggaaaacagtatggcagttcctcaaaaaattaaaagtagcatgatcatatgataaaatatacttctggatatgtatatccaaaagaaatgaaaatagaatctcaaagacatatttgcacactcatgttcattgcagtatttttcacaataacagaaggtggaagcaaccctagtgtccaccagtggatgaatagataaacaaaatgtggtatctacacgcaaggaaatatcattcagccttaaacatgaataaaactctgacacatgctacacatggatgaaccttgaggacatcatgctaagtgaaataagccagtcacaaaaagacaaattctgtatggtttctcttatgtgagtttatctaaagtagtcaaattcagagaaacagaagacagaatgagagttgccaggggctagggaggggagtaaacccattgttgttcaatgggtacagagtttccactttgaaagatgaaaaagttctggagatctgttgtacaacaatgtgagtatgtttaacactgctgaactgtacacttaaaatggttaagatggggctaggcgtggtggctcacgcctgtaatcccaaaactttaggaggccgaggtaagcacattgtttgagcccaggaatttgagatcagcctgcgcaacgtggcaacaccctgtctctactaaaaatacaaaaaattagcctagccggacatggtggcgcatgcctgtagtcacagctacgtaggtggccaaggtgaaaggattgcttgagcccaagagatcaaggcttcagtgagctgtgatcatgccattacatttcggcctgggcaagagtgagaccctgtctcaaataacaacaacaacgacaaaaatgatcaagatggcaaaatttatgttgtatgttttttaccacaattaatttcttttatttaaaactgatcatattaacaataaggaggtggttagtgaaggcaataagagcaagctcagggccttcgatgatacagagctgggttgcagaggatagggaatcaatgacgggtaaggaagtgaaggtggatcaccaaccttttcctcaaacgcctcatactctgtttccagatagaccacctggttgctggctgattcttaattttgttagatatgttaacaccatttgctacagtgctacccaaccttttgccattgtggtatgcatagagaatgatatttgtacaacacgctgaagtcaataggtaggaagcaaaagcagctggaaatatcagatatcaaggattcctgatgctccagggaacagctgcccaggagcgagaggaccgtaccctgaagaacctataaatcattagtggctgagagttgaaaaactctgcatttcactcaaatctgttgatgctacctaactccacttgaagccattctttcaatcttttggtcagagatgactcttttgaggtcagtttattattgcccataactttccatgatgtaaatctcaaaagatcagcttatagagcatttgatctgctatttctgaaatagctacttacttttataataaattcttacttttataataaatgttagactttttataataaattctctgtcacactaagacataaacagcaataaaattggtttgaagaaatgaaatggctaagacagaacagtttagatcaaggtttaagccaagaagtcacaacagaggctcagaatttgggaatatcaattatcaacataaaaacaacagcctaggaatctagaagtcattcaaaaagtagaaatatgggaggccaaggtgggaggatcgcttgagcccagtagttggagaccaacctgggcaatatagtgagaccctcatctctacaaaaaataaaaattatcgcagtgtggtggtccatacctgtggtcccagctgcttgtgaggctgaggtaggagaatcacttgagcctgggaggtcaaggctgcagtgagctgtgattgtgccactgcattctagcctgggcaacagagaaagaccctgtccccccaaccccccaagaaagataaagatagaaacagccaactggatttttacagtcaaataagtataggtgggagattcaaaaagtcagttcaaaacagaagtacctgttagaattgatgttctcctgatatgagtggaattctgtccctcaaataggtacgtggaagccctacgcctcagtccctgtgaatgtaacttgattggaaatagggtctttgtagatgcaatcaggttaagatgaggtcattagggtggactcaatatgactgttgtcctcataaaaagagaagagacacagacacagacacacagagagagcactgtgtgttgatgaagccacagaccagagtgatgtggtccacaaggaacacccaggattgctggcatcacaagcagctggaaaagtcccatggagtagacccccctcagaacctccagaaggagcttgccttgctgacatcttgatctcagacttccagcccccagaaatatgagacaatacatttctgttttcagccacccagttttttggaactttgtggcagcggccctagacaactaacacacctccagagccttgttctctggactcaagaagaaacgatcccttctaaagctggcaagtagcctggctgagagcatggggcagcagggaacaatggacgtacccttggaatcctacgccagcctgcgccaccctggagcagctttctcagcagttggagttattttcttctgcctttctttgattataggaatcaaaccccatcatagtaaaattttatagacttgagactttcttagaggaaaggtttctgtaagcccttccaaaatttcatgtcatgtgtgattaactcaaaccagaaggacattgttttagagaccttttctttgcctcatccatgggtactcgggactgttttgttagcatgatctctcttaccatggaacaaacatgaaactacacacctaagattgtccactcgagtctacaatggtgggttgtcggttgtatcattctttaattgtgctaattcagaattattttcattctagtttccacagcactctgcaaggtaacccttcaattaaaatatatatatatataggccaggcatggtgactcacacctgtaatcccagcactttgggaagccgaggcaggtggatcacctgtggtcaggagtttgagactagcctggccaatatggtgaaaccctatctctactaaaaatacaaaaaattagccaggcgtagtggcgggcgcctgtagtcctagctacttgggagactgagacaggagaatcacttgaacccaggaggcagaggttgcagtgagctgagatcgcgtctgcgaatctccatctcaacaagagtgaaactccatctcaaaaaaataaataaaataaaatatagtgggatttggagggaagatgtgcccaaacaggagagaagctcacacttcaactccaactgaacacncacccactgcttaactgtaaggatgctattttattttgtttcctttaatnncaagagnnnnacaaanattgttnaaganagcatgnagcttactgcactgtaccattgaaaacagcaaggatgataaggaagagcaacctcgcagcactttttctttcagatcctgtcacaggcagtgggaaactagattgcaatggttctaagagaaaagcatgacagttttcagttttatgcggtgcctggcagttgttgaattacaaaaaggcactcttatagttgtgttaagatttgttgtgaggagttagaatagtgaggtttaccctgtgtggacctacacattaaataggaacatgctggagctggagtcattgccctgattccttttggactcaactttcacacagtccgtttggatcttttttcaagtaattcatgtacttttccgttcttctggaagcatcctcacaatctgctttatcatccattcttcaaatatttgagtacctaccatgtgtacaacgctaggccaggtacaataataagtaagttctttgctcccatggagccttctggtctaataggggagttggatgtaaatcaagtaattaaaataattcctaactgtgtaaagattgagatgagctttcttcagaaaataacatcatttaaggagagacactcgtagaagaccccgacacaaatggtggtcaggggtgaggatgaagaaagttacaggagagctaagttttgcaagatgagccttgtggacttcacggaggtgtggtgggtcaggggaggggagcacaactccagggagaagggaaagcctctgtgcacccagaatgcaggaagaccagggtggagtggtatagagtaagtgtggaaaagtcagcagggcatgaaccatgcagtgccttgagaaacacggtaaggtcatttaccatttgttttttgtttgtttgtttgttttgagacagtctcactctgtcacccaggctggagtgcagtggtacagtcttggctcactgcaacctctacctccaaggttcaagcgattctcctgcctcagcctcccgagtagctgggattacaggcgtgcgccaccactcctggctaatttttatatttttagaagagacggggtttcaccatgttggccaggctggtcttgaactcctgacctcaggtgatccgcctgccttggcctcccaaagtgctgggattacaggcatgagccaccataccagccagaacatttacctttaatgtttacctttaacctgagccactgaaagcttataagatgggaggcagtggtgaaccagagagctgtttggaaaataaaatcaagagggacttggtgataggttggatttaaagacatgagtgagagagtctcaaagatgattcctggtttcaggtttctacagttgcatgaatgaaatgtcattaactgagtnaagaaacactagcatacaaccgtatttgggggaaacgttcatcaactcagccttgggcatgttgagttggtgaatctttcaagatgtctgaacngagatgcctaatagttgtgtataggtctggagttcaggggagaaggatggctgcctagagatataaatgtggacattcaatgtaattctcaattatttattcaggtgctgttctagacatgtggcgtaggtcaatgaacagaacagncaaaaatgactgcctcctttgagcttcagtcctagtaaagttctccatgttttcataatactggaaggtgtaaatctggatgagattgtatagggtgggaagaaaaaatatgctggagccttgaggaagagcaacatttaataactgctggtgtttgcctttatggtaactcaacctgtctctgttttcctccttgtgtattttcactttcagttccattgcaaatagtcaacattttctgtatctcaggttcaaaacttgatcaataagaacagatttggtcgatccagctaatcaatatgattccttttaggcaggaatttcatctctgattgcctcgcaatgctggccagtcttatagaaagtccctgtctttggcccggtttctggctcaaccagctttgacaagggtgatgagttactttgtaccataaggcaatttatnaggaagaaacaccttcataaaaaaaaacaaaaacaaaacaaaacaaaaaacagaacaggcagacatttggagaactcacctgcatattacagagagatttaatatgtttatctattgcatgccactgtaatttaacagtctaggcaaaggtaacagaaaagaggagctgagataatccagctcctccccaaccactcattcaggaggaagcagatgtgagtgccattgtagggtcctgagggagtattggttcccatttcctagcctgcccctcctccccacgcttggtcagttacaccttcatatggctcctgttctcactttacctctctgctttatgataagcgcagataaaaggcaagagacacagacaaagcgaaaaagaaggaagagggaaggaaatttagaagggaccttgcaaacattttccttccactgagtggtccacaggaggaaacaatgaaaacttctctctcctttgggaggccaaggcgggcagatcacaaggtcaggagatcgagaccatcctggctaacatggtgaaaccctgtctctactaaaaatacaaaaaattagtcaggcatggtggtgtgtacctgtagtcccagctactcaggaggctgaggcagtagaatcgcttgaacccaggaggcagaggttgcagtgagccgagatcgcaccgctgcacttcagcctgggcgacagaatgagactccgtctcaaaataaaataaaataaaaataacaaaaaataacaaaaaacaaaaaaanaccttctctctcactaacaaagagatgtgattttggagaaatgtcttccctctctttcagttttaccaaatctaacttgggtatctttagatccggtgtgccctcttttctttgatgacacgagtcctaactcctatatattgtcttatatgattgttttatgtcatccaagctggcacttatatcatctccctggtctcataggcatttaaatctgggcccctgcactagaccctgacaagatctcttctagatctaaaattctcatccgggaaagagtcgagagctgtcattactgggacacctgtagctgtatacttcattatgacagatctgttgtttatgtccagtgtctcttctgattttggggtgttcatgctgcatggttttctaatatacgtagttctaatatgttctctgttcattactgcgaatttctgctccgacccctggccacctacacaaaccgcaatccccagcataggcaacatgttagtaaccacccggagggggcgactgcaagccatgatgaagagctgggtctaccatgtacctgacctgtgctgcaagctctggcactagagctggggtcacagggccctagataagtaatctcctctgagaatagtcagattttgtcaatacatacaattttctcttagcagaaatcttggttgtattaataaatgctgtcattgacagcattgctttgtatttgtacatggatcaaatgaaagaataaaagtcaggaaagtgatgatattcattccttgccatttacttaatatgccatcaaactttcccactttcctattattttttcactctgggagtttaggaaagggaattatatgactacctggattctcatatgccttcaacatatttttaactggatcatttcatcttactctggatttgtgcctaaaatctcagtgctaagtgctctatcttctaaattgagattccaccttccaaagatgnactggtggtcctcttattcctgattatcttcccagctcacactcccccagatgaaataaggagagtgctgtgggaggaaagttgtggtagaatcatcagtagcagtgtttacggagagagattattcctcctttgccaaagcgcaagagaggacttccagagaatcacttgtaggagtgaggagtaccagtcaaacagaaagagactggctcttctatctggaagcttggaaacccgtagctctactcagttcagccactagattacagcaagctgagttggttgtgaaaggaagctggcatcttccttctgaagaatggtgcagatgggggtagagtgaacctgcccttgcgtgacatagacagttctgaaagtcagaggccagccagagtacagctagcagtagcttgtcaagaaaggactgtcaagaagaatattttctaagattaatgctgggctgggggaaccccacccctttctccttctctgccacctcagacagtgcagaggggagctcagcggtgcccatgacagatgttagaggaaggttggggaggcagagggctgcacaggcagaggccaagccaagacatgctcattcaggagaggattaaaggaaggacctaaggaagacaagattctctttctcttaggacgtctttggctgcggaagaccagagtaaaactctccttatacaaaaaagaaatcacacaaattatatgttaaccctatcaacaaaggacataagtgctgaatgggtgtagatccaaaccaaactagaagggaggtcagtggtttcctaagataataccttctgctcctaactctccactctgaccccaactttggaggagctacacccaaaagaagaagatgtggagcaatgaaagaaaagacagttggttgctcaccctgtaatctcagcactttgggaggctgaggtgggtggatcacaaggtcaagagtttgagaccagcctggccaacgtggtgaaaccctgtctctactaaaaatacaaaaattagctgggtgtggtggcgcctgcctataatcccagctactcaggaagctgaggcaggagaattgcttgaacccaggaggcagaggtcgtagtgagctgagatcacgccattgcactccagctctgggcaacagagcaagactccatctcaggaaaaaaaaaaaaaaaagaccatgtcccctccctagctcaaaaccatatgtcatactgtcatgtaatggggtgaggaagaagggaggtttgaatcagatatgagactagactgcactgaagtaaatttttaataaccaaagttgaccaaaaagttagggaattaactagggataacaaccagtcttttatcctcatttataatccaactttcctataattgaggatgactgatttcactgttggccttcccttacattccacttggagcattactcagtgagaggtagggcatccccaggcacagtgagaggtgagacattcctggcattctgtcaccgtgaaatttccagtcctcatcttgtgttttctgtaattcagctctaaagccagccatttcttcaaagagtgctgatttgttttattggagaatggtatttagggaccaaaancctagcactaggtttctcattgtttccagtatatcagtgtttctaggctctctcagcaagaagagcagggaaatacatgtatgtgtactaactcaagtacacacatacatgtatttagctagttttgtatatatttacctatctatgcatgtgtcttacataaccatagaaccatctgccttgataacttgtactcaggtattaacaatatactaattttgttagattgttactgggacatgagtgcagtatatacatgttattttctttaatgttatccctatttccacttgtttttaataaaatatgtggattaatgtgaattttcatagtgtatctaataagcccttttatcaacataggagacatttaaaaactattcatttgaccacagacatacacaaaaatgataattggaaatgcatgattgtggccagaaaagaaattacttaagggaataggacactgctggggtccaagaccccaatgttaagactgcattccaaagtacgtattttccatgcctctaatttaaagtgtcatatttatttgatagtaaataccattactgatacaacgttacatgcacactaagtgcaacaaataagggaattttctatagaaaatgagagccagagttcataccatcagagaaagaagttataaatgagcagaggggagatgccacaggagaacctgtggggctagattaggagtatcagtaagaactcagcttattttaagagagaggataggtaggtagttacatgcatatatatatatatacctgcatacatatggttggagggttatgtaagatacatacgtaggtaaatatatacaaaactaggtaaatgtgagagagtgtgtgtacatgagttagtatacatacatgtatttccccgctctttctgctgagggagcctagaagtaaggatatactagaaacaatgagaaacctagtgctgggattttggtccctaaataacattctccaataaaacaagtcggcactctgaagaaatggctggctttagagctgaattaaagagaacacaagatgaggataggacattttgtggtgccagaaaataaagaagacctaaaaaggaaaaaacaaacaagcaaacaaacaaacaaacaaaatagatagaagtatgtctgaagactagagaagccgactgaaagtgcccccagtggccaaagcaggaagactttgagcaatgaaatgaataaggaaagtactgggttataacccaaagaatacaataaatagtcacaaatacatatttatacagatacatgattgaataaataaataaaggaggagggacaactgattctctcagaataattccaagtaagaaatgtagagaaatagaaataatagaataataaatgaagtaaacagaaaattgccattaggaaactattgttgtaattgccacagacaagagctgatgaataccaaaattagtatgtgaaactttaaggagaaataaaacatttgcatggcctcaaagtgtcctctccaagtctttttttaattattgtagcagctttaacatccacccacaaattctttgatactcctccttcctaagagataaagctttaattcttctaagtgtcagttggagtcagtgacttgcttcaagccaatggagtaggaaaagagaaacactgaatgtaactttataatgcagaaacctggcagacatcaccttacccaagtaaggacagttaacctcaatgtcatatacacactgatatgacgagaagggcgtatcatctccatggtatttgtcccaaaaatgtataagctcagtctaatcatgagaaaagatcagacaacccaaaattgggggacactctgcaaattacctgactgatactcctcgaaaccatcaaagtcattaacaacaagaagaattagaggaattgtcacagccaagagaaacttcagatgtgacaatgaaatgcaatgtggtttccaggatgagctcctggatcagacgaaagtcattcgtgcaaaaacctgggaattcccaataacttctttagttaaaagtatttgaccaaggttaattttgtagttttgataaatgttctatggtatataacatgctgggtcaaggtatacaggaactctctgtactatctttgcaactctcatgtgagttttaaattacttcaaaatagtgtttttgtaaacaggatgagaattttctatggtaagctgtaataatgcaacatagaaattagctcctgtgttgccttgttcnaaagnatatgtacatctgtgtagctttctgccatgaatacgttgtatcaagtttatactttattattcttttttgatcttnaaaaagctgggaacacacactttatcaagagccaaatgctttttgtgattttgtttnctaatagaaaatctgatgtaacttgtcattactcctctttcatctttgccagtaatataactcatctgaaatcggcagacttgcttgtgctggataatatagatgattccatattgtaaaggctctaagaaaacaataatagaatgaagagaaatattacaaatgatcctcttctgcctgttgatgggactccgttagtgttgccttcttgttctcatctcatcaccattggattatcattgtattgtgtgcacaatttgaggccacagggccacagagagttgtgcccttaattaaaaatgcatcatgctagtaggggaaaagcacacgtaattagaaagttatagtgcaaacccctgagcaaggtgcaacagagggagatgtgactaaatgcaaggtgcacagaggaagatgtgcctaatttggtttagaggcaggtcctggaagatgtgtctaaaaatgatgcataaaattttcttaggaaaacgagagaaaatatcccatataagaggaataacaggcacaagattcacaagtaaaagtgtaccaattgcttgaaagtgttgttttggtgtggcagtatacaacagttttagaagctcaagctctggaatcaggaaaattgttgtcaaattccagttctgccacttctagctatgtgacttcaggcagctaattgaagctccatggacctgtttcctcatcgatgacattgcataatatggagctaattatggtaccgacatcagggagctaatttttagggttaaataatattagcataaagtgttcagcatcatatccagcacgcgtgtttactgcttcacctaccagttgttattctgagaaatattcctggagaagatggttttggaaggatttataagagttaaataagtgtattgcaggcatggggagcagtatcttcatgtgacatccctgtgtgaggggggcacagacctacaggttagagcatagagggcaaagcactggaagacccgcttatgccatgctaagaagattagaatttcttctgtacataattgatagtctttgcattactttaaggaccagagggacataaaggtatttcttttctagatgttctaaaagattggtttaaagggaaaaaactaagacagaaagccagttaggaaggtttcaggagtctcaacgagatgaaatttggatgaccaattgaccattggattcagcaaaatgggaaatacgctgactttgccaagagcagccccactgaggcagggggggacacctgagacaggagaggctggaagaggggtgggaagcgatataatggtgctgtaagcatctctttgaagaagcaaccttaaatagcaaaggaggcatgatcaggagaaatggtttaactgcctattttcgttttcatttttacaaccaagagtcttgagaaaacaatccaatcaacatagcaaacgagaagaaattggggaaagagggagaggttgagttgtctcagattagccagagggaagggactcaagcacacatatgtgtcaccccattcatatgggtgaagtcctcctcctgtagagcaagcagtcactgtggcatggtgatagctttggtttgtatagcacatttcccttgagtaattcaaattacaggcatattgctatctcacttacacgtctggcatttatatgaaagactaaagatcctcattttttatttgagaacactgaaatccaaaggggtgaagagattataaaactgaacaaaagtttaaatccatgtcttcttactttgttttttactgtttttacttatgctatctctgatgttttataaattaaaaaaaagagaaaaattacatgggagtgtatgtgcgtgtttgtgtgtgtgtaagtgtgtgtgtgtgtatcctgaattccctttggtgtgacatttttccctagcacttaacgaagaatcatctcacaataagaaccaagtatctagttgacatgaaactctaatataatttcctctgaagtggagcaaaaaagatatatacttttttttatcacactgagcatatttctatgactcagtaaattctctgatggaaacaactgtgtctcagtggagtaacaaaggacagatataatataaaatagagatgttntgggacctagaagaataaacctacaaaatcaatctttattcatcaccactgtctctaagaaattattaattacattaatgggatattaaaggacaataatatgagcaaaatgtgctcattcaacataattaggtattctggatttgaatagttttttttttcttttactaattcattagagttgttggttagcttagccttgctgcaaatattatctatgttgttgcacaaagtaaccttctgaaggacatttaaagcttgtaagatcttaagggttgtgataacagcatgaaagtaaacattttatgcacacttcagggatatcataagtggcaggaatgcaacagtctgttataagtgctctgctgagctcccaaaccaacaaacacaaaacagccacttaattgcccagtgcatccgcccctccgaaaacagagcttctcttcatacttacttaagggacatgctgcttggacgaatgagtaaccagccaggatacagcacatgcagttaatcagctaatgttatgaacattttggatgtcagctatcacataaaaatgtagcttttatggcccataggctaaccaagaaatgagccctccaatttctatctgtggcttcagtcgatgcgtccaaataattgaaaaccccactttgaccttggatatactgggtgtattaaatatttttgtaaaacggggaaaaattagtgacttaaacatgttttttgatgaaacgtttgaaaactgtacagtatcgctgtcttctatgcactaatttgacttggttgccttttgtccacagcagaaaagacagtgcccacccacctgaagaaattctgtattggatttcacaggccagtgagggatattctttgtatttgcatattaaggttttataattacacagaggtattattaaaaaccaacatgtgtgatcattattccagatgccccatctgttttgactgtgcgatttgggttgtaaaatcacatatacaaattagcaagagaaactcccttgaaaaataatagtttacgccttagcaagactctatgttctgaattgctatgtcatatatatagatacagaaaaaatagctaaagccactgtgtaaagaagcaaagagacttcagccttaatttattcacataacaaagaggagggactcctttcttctgattgaagataatgtggttatttttcctatattgaacaaccaaaaaatattctacagttacatcaactcacaaatctatgcacagttttcaaactaacacggactagatttcaaaatgagaatctcccaagtctgaatgttttttaaattgttttcttttgggaaatcataccaaaaaattacaaaaatttttatttaatgtaattcttcaattcagttattcctcacagcaatcagaaaggaactgaaactgaataatgtttgaatattttaacagaagaaagcacaggctgaaatagggaagatttaagcgatgaggcagaacacagtatgtgtcgtgcagagcaattacacccgaagtctcttgagaagtcagggaaagcagaggatttcagccgcccgatgtgtaatgatatctcagccaggcagatgctgagccgtagaatagcactgagcttagacgacttctagttttaaagctcaaggtttgaagtttgtttctttgattctaatttcattcttgcaaatgtgcttttctttaccatgtgaatgtgtgacaaagctatcatttgatctctcggttgcagccagacttcatgctcagctgaggccattataaacttcctttgtactgttctttacattagatttttcaatatgagaaataaaatcctaggattctctacactgtgttcttttccttcaagaatcatgatcttaaaaacactttttaaatggacatttacaattataattactttatgttaaaagatcttcttttcttggtatagtataatgatcttaaaaattcataaccacttcttataatatatattattcattcatttgcatcaaagtaaatttaatcttgcaaaatctttgcttaaatgcaaaaaaggggaaagaatttggataaaaggtagaataccatttcattacattttaaagaaacttaatagtggcttcaaaataacatgtaatactttgtgtatcatggtcacctcatcccccaccaccaacacacaccctgtctactcccatcttactctttcttcctttttctgtgtctgtagcttggtcaaaatcatgggttgggaaagacgtcaaagttttggtggctgatagtgccgaagtaagaatggaataattttgcttaactgcatcatcttcccttttgaaaaacttttaactgaactctaatatacatatagaaagaggtatagtatacatttttgtaaactgaacacacccagcaccctgctcaggaaatattgactgtgccttagaagctcctttccatactccctcccgctccccgccacccccacccaggacgaccactctgcagacacccggcagcacacgtcagtgccacctgctggtttaacttcctccacgtggaatggagtacatacaacagtatgtactctgttgttgcgtctggctcccttcactcaatgttatgttgtgaaatccacatttcttacactgagatgtagttcactcattcaaatatttaaataaacccaaatgtattttcccactcaactgctagtgggcatttggacagttcacattttgggacttttaacaactgacactgctgtggccaagggagggcttgtgccttgtgggtgtgcacgcagttcttgcaggcacgcacctaggagaggatccacagggctgctggatttacctgtgctcatttttagttgatgctgcttctctcttaataacagcaaagagtgcatgggaactgctgtccagaagaacaagtccttgcttttaggaggtttgattcagtgcctcagccatgagattcaaaatactcacccttcaagcagccgctcatccttctctgtgttaactggcccttaatgcacaactgtagggcaatgatgccaggtttgcacaatcgtgaagaaccttctgagtctccaaaaagagcttcccgtctgttctttgtagtttcacagcgcaatgaccaagtngtgctgagttctaattcatttatttacaaagagttctatttnctactccctagaatgtactatagaagtaaatttaaaatggtatcttgttcctaaggagcgtacaagctttggggagtgtaaatatctctaataccaattagaatgcgggtgaaacccatttttaaaatgtgtaaacaaggtgactgttggtatcctacatatgttaaacaatatattcaaggatattcgataaaatataacttgccatttctccaacatgtccctttataattttttttttttttttttgagacagtctcgctgtgtcccccaggctggagtgcagtggcacaatcttggctcactgtaagctccacctcccgggttcacgccattctcctgcctcagcctccctagtagctgggactacaggcgcctgccaccacgcccagctaattttttgtatttttagcagagacggggtttcaccgtgttagccaggatggtctcgatctccttacctcgtgatccgcccacctcggcctcccaaagtactgggattacaggcgtgagccactgcgcccagcctacaaaatttttatatctgtactttgtctaaacattcattagtttgtcaacgacagacatcagttcaggttaaacgacacaggagtctaattttattctcttctgtgaatctaaggacacgcatactccacactaacaaatttattcaatcaatcaatagatattttatgcttatctctctacccttcaggatatatctaaaatcttttcttttatttcccagttgtctgctggagagtttagcttagagttgagttccgccacctgatcaataccattattccttcactgaatatttgctcagctcaccttgtgtccagtgccatgccagccactgcccgtggaataatgagcagagccctgcatggttcctgtcctcaccaagtttacagtctcagggaaaagagaactgttgtgcaaggggtcacacaatatagtgtcaaacgacagtacgagtagggggtttaaggcattcccccaaaatttttgtccagctggatcctcagaatgagaccatctttggaaattgggtctttgcagaggtaaccagttaagttaattgttctggatggtccagtgtttggcatctataaggaaaaggagagggggattcagattcagagacacagaaacacgcaggcccagaaggccatgtggtgacagaagcagggatggggtgttacagctacaagccagggaactccgagcatggctgagaatcatgagaaacaagagaaaggcaaggaaggactctcgcctggagccttcagagagagcatggccctgctggcgccttggtttttgacttctagcctccagaatgctgagagagtaaatttctattgtgttaagctaatgatctagtggtaatgtgtgacaggagtcctaggaaactaatgtattgtggattcaattgacccgtaagaaattgcagatgttcaactgctttgagccacacaaacagcaatattataggattcatcctaccatgttatgagcttgtaagaggaaaactttgtcagagaaagcttctgtgaggaatttctagtggaactgaaatctagaaaacaagtagaatttgatggagttcaagagaaaaagaggaagaaaggggaacccagcagagggccagcagagggccggcagaggtgggggaggcacagaggttctgaggcagagggtgcagatgtttccaagcaactgaaggatgtgactggaacccggtgtggggatagagctagctgaggctggacaggcagggaggggccaaaaaaaggcctctgagcctagattgaagattgagattttgaccaggcatggcagctcgcacctgtagtcccggctactcaggaggctgaggcaagaggatcccttgagcccaggagtttgaggccagcctgggcaacattgcaagatcccatatctttaaaaaagaaaagtttatatttttgtttttgtttgttaaaacaaaagtttttttgtttgtttgttaaaagcagtgggtagccaggtgtggtggtgcatgcttataattccagcgactcaggaaactgaggcttgaaccagaaggcagaggttgcggtgagccaagatcgcaccactgcactccagcctgggagacagagcaagactccatctcaaaaaaaaaaaaaaaaaagagagagagaggaagaaaaaggcagtggaatgccattgaaaagttttaagacagagcgtgagaaatggcaataaccgcagaatgaagagcagattagaaagagctgaagcagatggagaaagcactgcaggaaatgcaggaacccagtggtgcattggccagggaggcactggtggggatgcaggcagtgggcaggcctagaggcttttaggaaagaactctgtgttctcatttcaatgatatgtcccaattaatataccatttgcccttgtgtctgtttgaagacattgaattctcataaaatatgcataccagtcctgatacatctagagcccccaagtcaactcttctgaccacgtgtgtctattctgctgcccgtgaagtctcgtggagtagaagtagtgccttcattttttttcttatttctctctttgctctataccttgtgtttccgagcactctgttcctatatctgccagagtggccattctattttattatcattgtctctttatagaactatctacccagttatactgtgggctccgtgagggcgagtgcagtgacttatttctttttatatcccctgagcttagcacagaacctgacctgtaagatttgatttataagtgacagtatcaatgtataaataaatgagtcagcaaatgcatgaattaacaatactttatttctgctctgtcttatcacctttggcctgatatacctgccttacactctgatctgatctaatagattaactgctttttctacttctgctctatttaccactgacaagtatttttctactagttgtatcaacttttagaaaacaagactcttcctggtaaatctctttactcgtgtgtggaatcatttttctaactgtgtacctctcacagagatgctactactttttgttttgtgtctttgggagctaattccttaccattgtcatcatcataaccatggaactgttgagaacgtgccattttgcacacattactgcgttgagcactccagttaggaagggaggaaggcaacgaacagcagtgactccttctccaaataagattaccatctaagacagaaaaaccaaaggctacccacaaaatcaaatcagacttacaaatggctaaggtaaatgtttatgttacacaaaacaaaaacataagagcgatttctgcagcctaataagaaaaccagaaatcaggatgtataatctcatcatgagctcacaaccaataggatagttgagtcatccatcatattgaaatgggaatgtttccccaaaaagaagccctcctgttctccttagccaatttgtatttccacatataggagggatgcattgccatctctcttcctgttttaaaagctctcttggcctctaattcttatctttccataattcctaaaacaaattatagagtaaaaccatacccaacaaggaggtaaatgagtggatgatttttgaaacccaccccccaaatccctccaaaaaggcagcctgaattacatgtactctttgaaaggcatcagtgttaaaagatgcagtttgggaagccgggacagaagacagggcagacgtggggaagggtgttccactctgcagaaggtcctggagaaggggagaggtaaacagaagtaaagagggactccaagagacgtcggcactgggagagaaaaagagagcataggaaagtatagcatgaattctggaagccacagtccacctttgtagtatcttagttcagccttgttccagtttatgatgttaaacctgagcttccctgaaactcaatttgtagaaatataaatatacactcttaaataaatccatttgattgtcattcctcacaaagatatcatatggcatttaagttcgtcgtgtccaagcatgtttgcagccaaatggaaaactcgctgcatggaccaaatgtggtatttgtgttttgagcttttctgttttaatctgctaggcgacataaccaattttcctgccattgtctctctttacaaatgctactacttagtatgattctgtagttaattttttttaaatttttcatggtttttaaaaatttttaattctagcgttaggcatgtgtaaacaaattttattgtttaaatatgcaagtaatagtaaaccattaaaaatgtctgagatgaaaagagacttgtctttgggaataaaatgacataaagttttccttgaatttttttctggtttantttttttgttgttaagttttgtttttgttttttctgttaactcaaaagttttagctcatagagcaatttatggcgggcaagagaggtatacttggtaggcctttgctgccatctactggcatttcatgtaattacttattgcttccattctaaaactaattttgtgtaacgtatataatagtattataggttcccccttttnttcttctttccaatttttctttagttaaagcaaagtgaagcaaatgcggacaccaaagaaaagctccctttacgcctgcgaatctttgaaaaatttccaaacagacctcaaatggtgaaaatctcaaagcttccttcagattttacagttcctaaaatcaggtaccaaaatacttctttaattgcatatttcacatatattgtaaaagattcatcatcttaaatctgatttaacccttcttttaagtatatgtgggttgggtttctttctattttattgtcatctttctactacctctacctctatttaaaataaataaataaataaacaactttgactcttttaagcaatgtattcacagaagagtcactttataactgcagtagcctaagcaagaaggctgcttcccatcgcttctctctgggatgactcctcgggaatgggacatcccaggaaactggagaagctgctcagtgttgcagggatggcaggtagacatcaatctcagaccccaaccgtatgagactaaaattgcctctgtggcccttttattttcaagcaaacagggaaaccacagctggcccagtttgtgtactactacagtgtttatggagcctaccatgtcaggttaccatatataatttgccttccactttttaaaagaagaaccgtggcgtttttccactcatactgatttgattttctaccacgtttgccctccagttaagcaaatgcataaaagatgctttaaacacctatttttaataataaaaggaaattcttctagttatagctcaagaaattacaaacagaattacagtaatatgttactagctttaaaaataattctgtggggggttataaaagtatgagactatatatggtgccataatatttaaaattcgggtagaaatatatttgcatttctaattttaaaaaacaaaattaattgaatttggtaatttgaagagacgttacttgaattttcgttatgctttgaagatcagatgaacgcacttgcttttgagtcttaaaatgtaaaattctctcttagtcattcttatgataaatggacatttagagagagcaactaattcttccaagtcttagagggggaaaaaaagtgttttattaccacaataagattcaaaaaactgggctgggtgcggtggctcacacctgtaatcccagcactttgggaggccgaaccgggcagatcacctgaggtcgggagttcgagaccagcctgaccaacatggagaaatcccatctctagtaaaaatacaaaattagctgggcgtggtggcgcatgcctgtaatcccagctactctggaggctgaggcaggagaattgcttaaactcgggaggcggaggttgtggtgagctgagattgcaccattgcactccagcctgggcaacaagagtgaaactccgtctcaaaaaaaaaaaagattcaaaaaactaccatcggttactgagtaatattgcttgaaagttatttcaaggatattgctttttaagcaggagtcactgagtttcaggaagctgttgctaatgaaatattttagtcatgatctagttttctatgatcttttatcactgctttatctttgctcaagcaaactcatcattgcaaaatgctgtcacttgctcccttccactaataatgcctgatgcacatatacttcatttgcaaagggagagttttatgaagcagtttattgaccgccagcaacaggacacatgttgcctcctgagaagcctcccgaccacctcttcctcctcctgcgaccactccaaacgctcagcaattgaggacaacaaatacatcgaccaaaaagtaagatcngctgtttcgaataaccaggcttctaaaaagcatctatgtttgagaaaagaaccgcagagttctttagggtcctggaatccaatttnctcaagagaataatgaaaanaaaatgatttcatttgtaaaagatatcatgaaacctaacattttttactctatgcattgaaattttgcatttgcccgtgtaagcttttgagctgcgccacgtggcagccatgcgactttgtaaacatcaagagcctcccaaatctgcattttctcangtgcggaataaagggctgggatctccaaggctcagtccaggtgtaatgacctgccatctcctgcaagtacaactgcttatgtctacttattacatcagagaggatacgagagcatgtttctctaaaatcggtcgaaagaattgccatttattcaatgcttagtatgtggcaggaacttaggtgcaagagcttattcatgttctagtttacctatttctttatcatttgaaaagtagaagggaaaatttttttcagattccacagcaaagtatcagtagggaaccacatataaataaagcaaatttaggtaataattttaaagaaatgtactttttcatttattcacttttaaaaaaaataatataagggctgtgtgccgtggctcacccctgtaatcccagcactttgggaggccgaggcgggtggatcacttgaggtcagaagttcgagaccagcttggccaacatggtgtaaccccatctctactaaaaatacaaaagtcagccgggcgtggtggtggatgcctgtaatcccagctactcgggaggctgaggcaggagaatcactcgaacctaggaggcagaggttgcagtgagctgagattgtgccactgtactccagcctgggcaacagagtgagactccatctcaaaaaaaaaaaaaaaaaaaaanangacntagaatgtattatctaggtataataagtaaacatatttatatatacacacatagacatataaatgtatatgtgtgtatatatgtatgcatagtatcaaactatatatatcagcttggtcacataatcaatgtcagttcagttagaaaatgaaagattaataattgtcctttaatattaaagagattaaacagtagagtttgcaaaaacatctagttcacttgcaagtattccactttggcattcctagagtgttttttattcctttacattgttactgccctttttcttttgcttatagtgaaatccatatttctctatgatcatgtgacaataaaatgccaactgtttcatcctccaggatcttctttctctccgctttctttcccttcccacagggatgaaccctttggaattccctcacctttctctttatctcgcctcccctttttctcctctttcttgcctcccctttttcctcctttttcacctcccccttttctcctcctctttctcacctcccctcccctaatgtgatactactcccgtttacctctcccccagctcccaggttctctgctgtagttcagtcatgttgctcttgctgaaaatgatgatgattttctttgcaggatgtctcttctttttcaaggatttccatgtgttgattttccttcagccatctagagatgctattctactatctttgcagaatccaagattgtaaatgacaagtctgatgccaatgtcattttttttttaagtaattagccttttggttggtagacttggtttgcttgtttcttcaggaaaacttgtaagattttctgtgtatccttggagttcagaagctttcttagaatataccttatatgtcttttttcatcagaactgctccaactagagtcttttcagcttgccaactaagatatttgctcagggaaatttccatttattttgcgttcaattgttactcttccatctgcttctctctcccctgggattgccatctctctatttttgttctgtgcttggtcttccaggacactaattagatttcaactgaggtcatcctttgcttctaaatttctcgtgaaaaattatgatatggacatatgacccttaactatccttaaaggcttgcatattacccttagctgtccttagaggctttacttttatcttttatttttatttttttagagatgagatcttgctgtgttgcccaggctggggtgcagtggctattcacaggcacggccatagtgcactatagccttgaactcctgagctcaagtgatcctcctgcctcagcctcccgagtagccgggactataggcatggccaccatgcctggcttttttgatttttttaaattaaacttatcacctctcttcttcacagttataatccactcaacactgtgcatggggcatggaagcagaaggtcatatgtcagtggataaacaagtgggaagatagtgccttggcttcagccagtcagtctgaaattgcagtttgcgtggactagaaagcaggttgttttgttttgttttgtttccaaagcagattgtaagccagaagtctgggctgtccttgtgaccacttatctccaggaagaggatagagagctgatttccattatggccaaagtcacaaaaccatgacatgcttttaaaaatcactggtaacactaccccactggaaacacaaaattgaaatatgagtttattttctctctaaaaggaaatgtgattgtttctcacatttcctgaacacagtattttgatttggtgaggacaaagataaaaagagtcttgagaggcatgtgccgggctgaggaggagtctaaagagaatgtggcccgcccctctgtgcgcccgcccgaagcgaaccctccccaggtacagttggaataacacgttttatcccatacaacatgtcttattgctcccacttggaaagtggaatgtcaaaaacatactttttcttcatcagaggaccctattatgggagtggcaagacaactgctcctgttactatccacgctttcttaaagctttgattcgatctaggttgtcagaaaagtagacttaattcttttcaggatccaccattttcagcaatagttcccaccagttcttggtgttcctcataagaccttaggatttgtcaaccttgttgagtgcccctgggccagggaaatctaaagcagaaaatccctcttcaaaggatttcagtgataaggatttaatggaagcacaaattgcggagacagcattctggcatttgtattatgaatacggcaagcctttttcatttttattaagaaacccgaaagccacttaaaaacaagagcgcataaacacagaacaaaaatccccgtcacagaccctaccaccattagttccccttataaagtatttttcagcgagcacttagaaatttccattaaaatcccaattgcctttggaaatcctagtcacagtaaagtgacaggcagggttcttcactctacgtgnaaaaaaaaaaccacccagccctctgcaaattgtgcaaattgaggctgtgtttcctcagtgactggtatgaaagaaaaaggacattagaggctggtttatcggtgattctcataacatgaacaaaggaacaataaaataaaactgttctcttaatggttatcttgtaccaggagaaacttctgacttcactatgctagagggaatcacctttctctttccagggaaaatagtgaagccccaggtgcctgaagactgcaagttcagggagaaaaaaacaacaactgacagcaaccccagaagggggctggggtttgatgaagcattgcagaaaacacaaaagcacattaggcaagattttctcccagtaattacccaagctttgaacagggatgagtaattcccatggcaacctcactttgcagaaaccatgtttttaaaaatagccctggtggatcacctttattatacagtttaaaatacctgtccttctcccagaaaacaaccagcaaacaaaaatctttaaaatcttggggctgacccagatcctaaaaggaaaagacaggaaagagtgttttcctgcctgtcgtttgagactccatagaaacgaccttcccaagtggtcatttccccatccctttatcctgctacctaccgttcctgtgagcaattgtgagaggttccttttcttcctgattcaaggattttgacaggggcttttccagtttccaagcagaaagaaatcaagcaacaatttctacttgtatttttcctccatattattgaaattaaactctagtttccttaagcagagtggtgattaataaagtgagatctgaacttgactttccagtaaccctgtaataaaagcagcccgctctgttctccgaagtgagacatccagtcaaggtcaaggtctcacctgtggttgagtctacagcgtctcactgccctgtggcctgaggatggttctgtgctgctgtcttggctgatgcaccatttcttatgtcagaacctgcaatagcctgggaagcatgagtgaacctcctggagccatgctaggatgagggctgtcccagcacctccacctcgtgcccctgtacttcccagcatagaatcagagcccacataataatgaacagtgaatattttctctgacagtagggcaatatctgcccaccctcttgtcagcctcctttctgctgaagcatatgatagttctttaataagccttcacaatgaacagctaacttatgtacagtaaagaggaaaatgatcattggaatgactatgttttgtttacccatttgattttataactcattaacattcattcagtcattcaacaagcacttatagagcacccgttattcatctgctattctctgggctgtgggaattcaacagtgagcaagatgcgaactgtaccctttagaagctcatagtccaatgggaggaaatgcacatacatgcatgaaagtaaaaagtattactggaaccatgagacagaaccccctaagttccccttacagaagaagtgtgattgattgataaaggaagaaagcaccccaggcagagggggacatatatgcaaaggtatgcaaagcatcaggtgctcgatgtttgagagaaattgggtagaagagtgattaagtggtgcaggttccaaccctcatcagtaaagtggagataattctcattttcaaggtttgttgtgagaatcaaataatgtaataatgttaaatacctagcattgcacctgggctagcatatgttctgtgcctctcaaaccagacaagaaaggttactgttantaaactgtaccatgaagtcagactcagcaaacagatttctattcatcagttaggacaaaagacttttacataaatttaaccttcagatgtaacagtctagttgagtgcaatatattaattactgatcaatttccttatggtacctaagtctgatttttttagagtttgatttttgttgatattttagttgctatccctattgtctacacggtgtcttcgaatctgaagaagtaaattaggtcattctggtaagatttactcttccgntttttttattgccatacagcattttataatgttctatatgattatagagtgcttgataatttgttcttgtaactcctcacatagcaagttactcagcctgatctataatttccaggatgaatgaatgtttgttcaccttaaaagagagagagagagagagagaatgaacaaangaacgaacgaacacagcctgntttcatttttagngcttttggtcttttgaaagaattttttttttaaataatggcttagcttctctgcagtaccattcaagatgtatctccaggaaaagagtgacagatgacatgatgctaccatgtaaccatgggttgtatctcaaagggaagaattggtgttcctcatgtcaaatatgaaaactctgtacacttctctctagcttaaaaatgctttagtagataatggattcccaatttagtcacaataatggaaagaaataaaaagatgcgtaagatttcatcatcactggccatcagagaaatgcaaatcaaagccacaatgagataccatctcacaccagttagaatggcgatcattaaaaagtcaggaaacaacaggtgctggagaggatgtggagaaataggaacacttttacactgttggtgggaccgtaaactagttcaaccattgtggaagacagcgtgctattcctcaaggatctagaactagaaatacatttgacccagccatcccattactgggtacatgcccaaaggattataaatcatgctgctataaagacacatgcacatgtatgtttattgcagcactgttcacaatagcaaagacctggaaccaacccaaatgtccatcaatgatagactggattaagaaaatgtggcacatatacaccatggaatactatgcagtcataaaaaaggatgagttcatgtcctttgtagggacgtggatgaagctggaaaccatcattctcagcaaactatcacaaggacaaaaaaccaaatactgcatgttctcactcataggtgggaanctgaacaatgagaacacttggacacnaggaaggnggnacatcacacactgggacctgtcatggggcagggggaggggggnnagggatagtattaggagatatacctaatgtaaatgatgagttaatgggtgcagcacacaaacatggcacatgcatacctatgtaacaaacctgcacgttgtgcacatgcaccctagaacctaaagtataatataaataaataaataaataaataaataaataaataaataaataaaacgatgcgtactataaaatcaaaccattttttcttcttactggtctttgcagtaaattctcaagctaaaaccaaatatctgtgtgtgtgagcactcaccctttcatccaacaatcatttttgaggaccattatatgcaaacagtttgctgggccctgagtaaacaaacacatctttctatattcaactaacattagagtaggaattccctctcaatgattatttttgcagaatatttaattgtgtaaccatttttaggttggaactatgtatcagctgtttgtattccttagtaaatgcgcattgagagtcaaaagtaaatcagtctaggattaagaattttttataggtaatttaaaagatttttaatcgcattttctgtatcatgcacaaaagaggtatcaaggagttaaggccgctgctaaatctatcttggaaatttacattttgctcttctttttaaactttttcctcttctgactacagttttacttataaattgtctcttctatcaagtgtcatgctagtctcagtgtgttgtatttgttgctgatgatggtgttgagtccaagtcctgcttccttgtgttgccacagaccttaaatttcgaatttctacacccataatcccagcactttgggaggccaaggctggcggatcacctgagctcaggagttcgagaccatcctgggcaacatggtgaaaccccatctctactaaaaaaaagtagctgagcatggtggcacacaactatagtcccagctactcgggaggctgaggcatgagaattgcttgagcccagggggcggaggttgcagtgagccaagatcatgccactgcactccagcttgggcgatacagcgagactccgtctcaaaacaaaaaaaaattttggaatttctaaattcacaaacaggttaaaagcatctgacatttactctagaatcacaagcccactcaccttcttttgtctctgcagatttccaatctttgccttcctgtttatattagatataatcataaagtcattttgtattttacttttgtaattaaaatctatctagcgtatatttatagttttgtttgtttgtttgtttgagacggagtctcgctctgtcacccagcctggggtgcagcggcacgatctcggctcactgcaagctccgcctcccgggttcacgccattctcctgcntcagcctcctgagtagctgggactacaggcgcccgcaaccacgcccggctaatttttttgtatttttagtagagacagggtttcaccgtgttagccaggatggtctncnatttcctgacctcgtgatccacccgcctcggcctcccaaagtgctgggactacaggcgtgagtctccgcacccgggcatttatagttncttatcattaagtactgtatattaaacctatctaaatttgtttcagtactcgaagatattgcttttccctcgatttttgcacagtttcattatttnatgtgtaagatgttggagatttgaccaagtgaccccaggctactgtgttagtggccgaaacattgtcataacattcacgtgcaaaaaaaaatcagatccattaagttttaaattgtttagtttctaatattacaagctgttcctatatatcaagttagttgattcatttcaaaataagcagtccctctttgtaatttttacgtcattcatatttcttattttcaaggactaactacattaaaatatataagatagaaaaagtaatactttaatattatgaaattaattcaattttcaaatatatttcaagcaccaacaatgcacagtgctagacaatgaaagaaaatgtaataaacaaagaatacattatacatatcctaaggagtttacaatctattggaatttacagaaagataaaaagatgtttcacacagggcagggatcaacttgtgctgggtgcctaggagtccaaaggaagagcgaagatttggtgtatgcatggcatgcacacacgtgcacatgcatgtttatatgaatattttaactcctacacatagtgtttttacagggttcagaacacatgatattgaaataagttacaataggaaatctttttttttgtttgtttgtttttttgagacagagtctcgctctgtggcccatgctggagtgcagtggcacgatctcagctcactgcaagctctgcctcccaggttcacgccattctcctgcctcagcctcccgagcagctgggactacaggcgcccgccaccatgcccggcttattttttctattttttagtagagacggggtttcaccatgttagccaggatggtctcgatctcctgacctcgtgatccacccacctcggcctcccaaagtgctgggattacaggcgtgagccaccgcacctggcctacaataagaaatcttgacccatcatctaaaattctcttcacaaaattcacattttaagatactgttcaaagcttcataacatgatattataatggtattcacctttatatacatccataatcaacattttaatcttgctgatgctcacattaatcaccttttaaaattttttttctaagcataatgtcaattccnagagaaagtttattttatagaaacatcttgccacacacatgatctgtcgtatgcactttgggattaattatgaattcacgggtgcatgaaaattcaaatacagtcaaatgagataccaccttcgacccatgcaattttaagtaattgcttattcccaattactatattttatttgggagacttattcaataaagtggctcgttacaataattacactacaactttctagcagaaatgacattcatcaagccatattcattggaccgtggttttgtgtcttatggtcatatgtttttataaccactttcccatttattattaacctgttaatatttgtaaagtgagaaaagaaaaatggagccactcaaactttttcccagttagaaacagaaatgtattgttaacattgtaaatcaaaaaagctattgaatatttataatacttgtggtgtgctttgtcaaataaatattgatggcttcattctgaaggtggtttctagggtcaagctatggttctttaggtctaagcattgccgcatcctaaaggaatctatattcaagataggtctgtggttacttctgggtgattgaattaatcaaattaatattctgattgattgagagttttgatggatcccagattttaaggaatcagtaagtgtaaaatataaaaactatgatcatactagctttaatgagttaggaaatgtattgatctctttcagtgcttcagagtcattattctgaaaatcaattatgcatatgtagaacaaacaaaaagttggttagtttcgattttgcattaggatgtgagaatagcaagagtttataatatgtgtaaacaaaatatttcaagtcaagcagataatattttgttatgatatagtttagaaaatgcttttttatactaagcctagtgacattttacatagacataatgcagtgaaaagatttgaacactgaagctgcatgatccagagttctagtaataattttgcaatttattatctgagtgaacttagacaacttaatttttatgagccttagatctttaatctggaaaaaagagataataataccttacctacctatgaggattagtttgaagattaagtttaaaatatgtatatatcacctcagcacagatcaaggagccaagaaatacagatgtagaagttataaaatgtacatttttaaatgtgcaatgggcatctataaaattcttcattcaatgactagagaataacattctaattcaagaacacatgtatgattttaaatgtaaccatttaccacagacttctaaatatatacacatcagtgtcctagaaaagatattatttgaccaaaatgtaacaaaatcngaaacctaataagaagaaaataaccacaaaaaaacctacacattcagaaatttaaaactattttcaaataagcacagaagaaacaccaatgaaaataagaaaatatctacaattaaaaaataatgaacaaaagatgcatcaaacttgtgggaagtagctaatggcatttttagaggaaaaaatttagccctgaatgaatatctagtaaagaaataaaggcttaaattaatcctccaagcattcatttcaagaatttagaaaaattatcaaccaaggaaacactaagatagcagaaagaaagaaataataaagagaaaagcagttattaatcaaatggggggaaagaggtgataaataatatcaacataattgaaattttgttctttggaagcagtaataaaattgataaatctactttaaaattcaacaagaaaaaattaaggagccttcttctataaagatgacagaggcattgacattgttttttaaccttgaattcccacatgaanaacaaaaagaataccaagatatcaaaaccccaaatccatagttaatatttaccataaaactcggtaatccacaaaccctaaataaaagcaagtgttgacaaacagccacatgatttgcatgatattagtgtctgtgggaggaaaccaagcttagttacagagcgtccaatgttcatggaatggtagaacatcttctttgctccattctgaaataaaaagtccaggagtggaatcaaaattgaacaggccaggaacaagagtgataaggataaaagcagatccagacaaaatttgtggggggaggaggattgggaaacagagcttggaaattccagaaagaaagctaccatatttgatcgatatattaaaacaacagaagaggaaactctgaagttagaagagaaatactaaactgtttgggaaaactaattttgtataaaaatgagtaaggaaaattatcaaagccaaattccctgcaaacgttattataagaaaaaaacagcagaatactgtctctcactgtagacaaggaaagcaacctanaaaaacgtgctctaaccagctcaaactgtagcatactattgaaaaattagttaaaagacattaaaaattacataaggatgaaatagcaacataaataaaagttagaaaaactcagaaatgaggtgccagaactgagaaaagttataaattaaagcaatgaaaactaaacgagaaggaacataaagagacctcagatgattcaaaagtgagaaatagaaaatggagaagagggcattttttaaaatcaaagagaaatgaagaaagagatgaaaatgatttgcaagaaaaatgacaaattgaagaaagtcaaaaatccgacatctgcattataggtgtccctaaggaagaaaaaccacagcaagagaataaaacgtatactaaacttttctgaaacataaaaagatatgaaactatatattgacagcacacacctaaaaatattggagtaaatgattaaaaacaatacatacaccagacaaatgacaggtctttaaattttttaaaaatatattatctagcccaaataagaacaagtgatttataaaggaaagaacgttagattatcatgagatttttcagcagcaatgctttatgtcagaagaaaatagagtaacatatataggatgcccaggcaaagaatatctgagccagatttttttccatcctgaaaactgactttcaactttcaaggtcagagaaactgctttcaagatctaagaatatgagtaatgtaagatacaagtaatcttgtttcaatgaacctgtaatctactaaaaaacatgcctcagataacctaaatgaactccaaaagggactcttaaaccatttggcataaaaattcacattcatatgtaacaactaactacataaggacaacatgaagagtgaaatatgtaataattatttgttctaatatggatagagtacagatatacaaatgaggaggtgaangggaagaagatatgcaaacccttaagatattcagtagtnttcttggttgtgtattttcattctgagactattgtctatgaaatgtgaaataaaactgagtcactgtngagacaatctaattctggcatccctggtgctcttgataatcaagagtgctctttgtggagaaaaaaggagatatagatatcatatagaaaagcttaggtaaaaataaaaatcctaaagaaaacgtggcatgtatacaccatggaatactatgcagccatataaaagaatgagctcatgtcctttgcagggacacggatgaagctggaaaccatcattctcagcaaactaacacaggaacagaaaaccaaacacagcatgttctcagtcataagtaggaattgaacaatgagaacatatgggcacagggaggggaacatcacacactgggtgctatcggggtggggggcaaggggagggatagcattaggagaaatacctaatgtagatgacgggttgatgggtgcagccaaccactgtgggacatgcatacctacgtaacaaacctgcacgttctgctcatgtaccccagaacttaaagtataactaaaaaaaaaaaaaaaaaatcctgggtcatgaatttaaattgaaaacatcagaataaacccaggcaaaatatttgaaacagtgaccaactccatagcagtgagcatctatgggctctagatatgttcttgaaacaccatttttccaattttccaataagtaaaccagggcttcttgaagaaaatggttgattttgaataggaagtgtacaagctgaccttggagcatcttgtcatactaggaacaaggaagctttgtcaaagactacgagggtcatataaaaagaacctacagactgaccaacttgaaaaactcccaatgagcacagtagagataatttgactaacagtaagaaatggtaattgacatggactgaaatccattaaacattaaataagtttaaatcttgagttcataatgacattnttttaaatagtaattggttaccttgagcgatgatagaaaatcaaccatgtaaaattaataagtaaaagtcgataatcaagcatttatcttggcttttccataccagttttgctactgggtaaccaaataatacaggagggaaagcacctcattataaaactatcccaactaataaatgaaaaagaaatgacagttttgcaaacggaatgaagtcaagggatctaagaaatgaacatcaagacccgtaacatcacaaaaagaaacacacacactctgttcctttcgatggaagaatctgacaccacctatggccttgccaaagggatgaaaccagagtctgtatgatgaaacctctggatccagctgccgattttcagaaaatagaagacagaagaacgtgttgaactgtaccaagagtgtgctttcagcaaaatccagactctctcttggctggnngtgcggtggcttactcctacaatcccagcatattgggaggccacggcaggaggatcacttgagcccaggcatttcaagaccagcctgtgcaacatgaagaaaccccatctcttaaaaaaaaaaaaatgttacttagctatatgtgatggcacacgcctgtagtcccagctactcagaaggctgagacaggaggatcgattcagcccaggaggtcaagactgcagtgagtcatgataattccactgcatgcactccagcctgggtgacagaacgaaaccctgtcaaaaaaagaaaaaaaaaaatccagactcaaaaactataggtcaaatggcccaggtttctctattttaaggggaaaaaagggtaacatgtagattaattagaagacatcgccaaaaaatcagcaagattcatacattggaatgtctttcaggtccaaattattcaatcacaaataactctatgggaattgagttgaagcttttgaattatgtgttaaagtatgtaattctggaaacgtgttttaacttagaagttcaagacttaaaatatctctatattgttgataatttaaatttaatttacttgttttgagttattaattctgtcaaagaattgttatctgaggcagttggatgttttcagtttccaaacaaaaccgaagaaaatcttcttcaacgaatctttgacttttttcaaagtaaactccttgtaattccatcattcatccccttcctttttattcccacatctctgatatgtttacatccagaatgagcaattactggctcctttcatagctgtaatttacaatttcagatctgaaattactcatcatgaaatggatctctgtattctttgttttgctaattatttcaggtgattgtcagtatcttctagagccagaagagggcactgcagagcagtgtagctctgagctctttatgtttagcacagtctaggaatgccctttgccaaaaaaaattcgcaaagctttactatagaagtaagaaaaaagaagggaataactagataatcactataggccttctttatttagtagaaaaaagaaagtatataacgatctaaatacatatctaggacatcaaatgtaatatcatatatatatgtataagtagatgaaagaagacatatatatgtgtgtgtgagtcctttccttcttttaagaacatcccaaaggccaggtgtggaggctcacacctgtaatcccagcactttgggaggccaaggtcggtggatcacttgaggtcaggagtttgaggccagcctggccaacatggtgaaacctcgtctctactaaaaatacaaaaattagccggtgtggtagtgcatgcctgtaatcccagctactagggaggctgaggcaggagtattgcttgaacccagggggcggaggttgcagtgagccgggatcacaccactgccctccaacctgggcaacagagtgagactctttctcaaaaaacaaaacaaaacaaaagaacatcccaaagctaaatctgatgtttttctattataatattgattctcctgctacctctggcattagttataaaaacagttctcaagtactctcctgtatgaattaatactgtattttgtcttagttcaaattcaggacaaaggccattcagtagccttttagcaatcttgtgcagggcagaaatcatttaggcaatagtgtaccagatattatcccaaaagtttttcctaacgtgaaattgcaggggcagtagataagtgattggattcagactctcatcttccactgaaggaaagacaactaacacatgtcattgtcctttcaagatgaaataagagttgtggaatctggagtcctctcttaaaactcactgtcacaggacctctgtgcctgggtctgtctcaggactatgtgtgttcttaggtgtctaaagagcagatgctccacgatgatggggccaccagctgagtcttccatctccaaaagcggctccaagcagctttccttaggagctgagctgtcaactcaagagaggttaaatccatgtcaaaagtcaacttttggccgggtgtggtggctcatgcctgtaatctcagcactttgggaggctgaggcaggtggatcacaaggtcaggagttcaagaccagcctggccaatatggtgaaaccccatctctactaaaaatacaaaaatcagtcgggcatggtggcgggcacctgtagtcccagctactcgggtggctgaggcaggagaatcgcttgaacccaggaggtggaagttgcggtgagctgagattacgccactgcactccagcctggatggcagagcaagactatcttaaaaaaaaaaaaaaaattatgccaaaagtcaactctttcaggaggggagaagtaggaaagtttcaagatttatcattggaaatagcatgtatgtgctaccaagaaaatggaagttccaaatagttggtatccactcttgtctccttgtccatacattaatttttctcctaagagttctagtgaataatgaaaccaattccggtttttcatttttgagcagaaaaatgaacaaaccacctttcctcatgagggggaaatcaccagtatgttcagtgatgctgatctttactacatatctgctgtaaaatatgaaaagtggattgctagttttgatgttatctaggttatcatttgtgactaaaaagacctgtttgttactctggttcttctccactatggctttatggacacactgactgacattatagcatttgttcatagctttgtttgtagaatggggtgctgtgagtttgtacctcaacatacctaacccttgttgaaaagaaactaactaggtgatctatttttactgaatacagagttttctgtcttctgagggttttctttcccccctcaactttgtttgacacacagtatgccctgtagggcttcttgcctggtttcaagatctggctagcacagcacttatacaggtagtttgtaccaaattaacttgccctgtgcacacatgtccacttcagtccttcacccagtaccgcctttgcctctaggaatcttagccttccttcaaggcccagtataacctccactttttcattgaagcctttgccgactagtccagacttgctgttccaccgcagagagctccagcccctctgatcttgtgttttctcctttacgtatcatctagcactgtgccatctactgcactgtacttgtcactgatgatctcatgtgtcctacccttgtcttcctatctaggctattcacttctagaaaccaagaactccaaattctatttaccctaaatctcctatggtcactaatatcatgctaagtgctcagtaaatctaatctattgatcaaaatgaacaggtctcaattactatcttcagtctgactactggagttacactttcctgttatttattatttattctagttatttattattgtgaatctagttatttatttattattatgagaacatagtggcccatttgcctgttttctctttgcccttcttctgcctgctttaactccttgtanaccttccaccaatctaaaatctgcaactagcaagccacacagaactgagcttggggagtatatctttgaatttacagtcagcatccaggatggttttctccttagttctcttctctggagtaattttatcatcgccaactaggaagagatcttgaatgcccatttcattcattccccacacctgcaagcaaaacaactcctaaactcattgttcagattagtgttaggttacaggccaagaacctgcagcagacataatgtggattagagaacattgcgctaatcaaagacaaataaggagtccagtatgagggtcaacgttaaagtctttggaaactgaaaattttattcagacgaaggccattatcagagtgaggcacagccaatgcaccatccgtggctgggacgtggggccccacaagtttgttcatggtgctgcccaaataaaagtaaaaatagacaagaattgtcaattgtacgatgatgctttagcgaaacatattcatgtttcatgttttaacaatgtcatctattgaatgatttgacagtatattatttcttaacattccaaaactaaaagtcatactcttatcattttcagcttcgcaagtctgttttcagcatcagggcaaggaaccttctggaaaaggagaccgcagtcactaaaatcttaaggtaacaccaattaatgttgacacatatttaaacacaagtgggattatattaatcgaaaaatagctttttgaaaaatgaattaatgttctgggcatcagcatatactttctgccagaaagtagctttgagtatctatgtacttataaacaatgtaaaagacatattgatgtcatttctaaaaaaaaattttagtagctcagtaggcctttctttcaagtatttaaacataaatgataagtataaacattgaccaaaaggtgaaaataacttactgttatttttaacagttttatatcttttgtgatatgtaaaggagaaattgcaatctaaaggataaatggtaaaagggtaaactttttcattatagttcattacactattaagagcacatttattaaaaatccngaaataataaaaagtataccaatcttagaatgcctatgaaaacatatgcttttaatgctactttctagaagatgcgctgaaatgaagtatcaaggaggaaaactaagcaaccgacttgagtgttttataagactatatgttatattatggagtaacatgatgagtataaaaacacataggcatttagaatatcatttattggtacatatattacatagacatatgtagatgacacaactcaagattgggagctggaggtaggatcgctcgcaaggcgagtggacagattcatgccaggaagccccctgcaaacctgcggtgagtgataaggagttctagctcccttctactagatttattactaggaatagacattaatgcattctctccagaatgcatattactataacttggaagactgtgtgagaaaataaagcttagtatatatgatctttaaaactattcttactttataatactacctaaaatttgtgtgcaaaaaaaatttcctaactcatcaagacagtatttctctcaatattggctgacgttcatgcatagagttaagtatgtgttggagtagctgtcataggatgaaaataagcatttctagggcttgtatattatcaccacaaaatgtgatgagataaacagacaaggtctcattgaaatagacaacctgcgccccaacttaggcttactcccgactggctggtaactccatcctggtattaccatcagatcatgcagtgaggctggccttgaaccacacatgttacatcaggatcatgattccagctgcacctacatcagacaggttcatggtagggtcctacagcaaggctgatccactcactaaaaagagaaattcaccctcaagccagaaactacctacaaggacatcataattctcagtctgcatcgagtatctgcctgccaagtcaggaactacagggaccttactcacctgtccagagccactgccttgcagtgtgaatgggccctctgtgtgaatgggttcccagagactttccaaggcagatgcagaaacagatactggcaaggaagagatttccagacctcacttctctaggaactcttccttaaaactgatctatcactttacctttttcaccctcctgtccttatttacccctctctcagtttacaaaagaaaggtgaaactctcactcatccggaaactatggcgtgtattaatctgggatgtaaaatcctctgggttgccaagcaaaaggaccgttcccacctggatttctccaaaaagagggtcccatggggccgtgccttattatttatccaggtgacagttttatggataacttccttcccaaagctatccaccttagattaggccttagaaataacatgattgtcacaagggcatgtggttacatgttatttgaattgaaaaataaagccagactttttctgacagattcagctgattggttcaacaatgaaaactgatttaataagtgcccaatgtcataaaattgaatagattggatggcgaagagtctccaatattttcttaaaattattttcaagcacaaacacaataatgctagaaattacattcttctatttaacgtggtgcaagagggatatagttgctgatttttaaatatacagaagtgtttttgttttttcaattatataatgtacatgagcaatatttttaagactatcagggaatatagacacctctaacttgcctaacaatggtgtagtttaggggatacccacagccaataaataatattttactgcagcccagcatactacagaatgagaggcagcaccttaaaactacctgagctatatcaccttttgaaactcaggaaataaaaagccataatttgaaaaaataaaaaaccttagaattttttaaaatacaaatttgatcttagaaagccattttgttttttcttctgtgcgaatggtttcttcattgcttctatgctagtttctgcatgaattaagggcaagtgcaaaaaatcaaactgatattttgggaatacttatcttttcaccactaatcgtagtaatagtaatggcaatgataccatacctgcattataatgtgatccatcagttaaaaagtcaagaatagattgggtccatgtcaatgagtgtttgtattgttttatactgtttttcaaggaacaataacagctaaaaggaaaaaaagagggagaaaaagaattttttttnnaaaaaaaaaaaaggaaaacatttgcagtaacctcaggacaattgattcgaataaccttccacaaaattgggaaggagaaaaagctctccaccactttttcagtgtctttttactgcctctagggactccgctctcctaggctccaaccttgctgtcgattaaactctgccaaggttcctggataacttagcagtggggtctcccaggaccatttgcattttgaaagaaggcttttaaaaaatgaaagggcttttaagacaaagaggatgcagattctgtcttttaatagatgggtctatcttgatataaataaaagaaatctggaagagactgccgttttagtgttctattcctcttaactttctaggtgttcttaatttcatagaattattgtttttagtcttatccaagaggttcctcgccagggaggtaagtaacagcagtatagggaggcttgaaaaagagacctgagctggatcattccggcaaatatcacaggttggcattcgtcctaatttgtgtgcagatagagataaatcacaaaaaccttaaatccagagtgatataagaataggaagtttttatgaaatattagcgtcttgagataactttccaaaaaataacaagtatttgggaaaaaataaagcaaccccttgacttaaggtttctggaggcctggctgtgtaaacagacaattaatttcaattctcagctctcttgtctattccatgggagctaggaggaggcgatcttcagggcccctgaagatctaatgtaggatactggacttggtgttaaatcccccctggctgggatcaggctctgcttctctgtaacattgaacacctcactaatctgaccctcagttttcttatctaccttaaaaagattgtctgaataatctattgaccaagcaaaatttaatacttattccagtaagggaaactgccactttgttttcataatgtctcagaagggataagtgagaggtggatatatgtaaggttttgggtttgggtttcagtgtttgtaggatggaggtctgtttagactgggcagaattcttgatatgagttgaggagtattgaagatgcctggtatggacaagtaatagttactgttctgaagacggcagtgctgtggttgaggaccagaaagagaaagcgacctgcctggtgcctctccactgctttccatgttctttcccccggctttggttcaacatttgagtaatccattctttacagcgagtttgcccttctattttcattctccttcttgacttgggtgatctaagagaagaaaaataaatggtgaccttatgttgtcctcttcacacatgaaatttattttttcttttgtgcctttcagatttttaaccagacacatgtattacctttaacaaacaaatacaactgacttttatagtggtttttttttttttttaagtttggcattgatccccattgggtagagtcttcacaaaaaatcctaatgaataccaactatttgtaggtgactagcttgaccagttgagtgacctactgtctggagaaaggggttggcccagcagtctatagttcagataaatgaatttgtgggaagtgcctgaaatcaacggtgaagttgctgactgtttaacagctttaccttcctggacaagtcaagtcatattgacgcagaggctcagctctcagccctggaaattaagctggagagctgccaggggtcttagttctcaggggggacccacatttccaaatatccatcttccatttgaaacaggatccacattattctagcagaaagtatgttatatacattactatgaacaggaaattccagcccacagaggaagaagtctttcagagaggaagggccctagagcctgaggtatgtaactcacaggtagaatcacccatttctatgtataatcatctttaattcatgttcttgtacttttgaaaattaattgatgttttgagaaatataaatgaattttttgatatatttgagaactctttgatgagaaaaaaaaaagtgggccatttttacagaacttgaggctactggcccatgctaaaatgcagatgattcacttggcacaaacaggcatctgagttgttgttttttaagagttatccgaagaggatgttccactacctgaattataaaatccaggattgctattaaatttattgttgttctgacttagactgtatcgttcagctccactaaataaagcttcttttcaaaaggcagtgaccttttctctcatcaggcgaaataaaaaacaaatagcaatgtactttcaaaagcatcactacctttgttgagatttcctctaattcaaagccctgaaatcgagcaaaaaatgcattatcgttcctgcaaggctgaatgaatagagaaatgttaaaataaaacaattaaacattttacattagtttagaaagttccatccttgtgtggtttgccatgagtacctccttagtgaggacaatatatttataacctttcgaatgctcagtctcgtatgatgtttgcattctgacatttggttttataacgtaactttctttgccatgcacttatattaataactgtttaagtcttaaggaatagtctttagtttagtgctacttactacagaaaaaaattaaacggatttaataaaatacatagagatagtttttctcttatcattttaaaagaatcatttaatgactataaatacaacttatatactacttacatatatatataancactatgtatttattaatctgcaattatgtaaatgacattagctggattttttttcctgaaagaaagtgcccctaagaattcacgcattcattcgagaaatatctctgtagtgtctcacatgccaggcatttcacaccatttgctttttcccatttggaaagttctccttttataattattgttccataggttagccagctattcagcaactttggataaggtcatattatttctctgcagctccgcattctgggcagaactagcaagacaccctgtacactgctgggagggcctgagtaccttacagcgntcccctggacctttgagctcagctcctcagtggaaagggaaggtgctggactgaccctacagatgagagaaagaaacgacagagacccttgcagcctagggtttgtgccaacccctgagatagcagcaatttttttcttagcatttggtatgaataaccccaagttttaagaaaatagagtatgagaatgaatgtcataaatatttatctttgcaattctatgaagtctgcctggttattgttggctttttcttaagattttcctgacacttaaaaactctgataagttagttgaaagactatttggaaataagctaagtagatagatgaaaattaactgtattcagacaaccataaactaaggagacaagatgtgtagatatatgtttttgcataaaacataaaatagtaaagatttaaaagtggcaaaatataactaaaacaaaaataatatattctaatcacatttttagatagtacctgatttgcggtaatcaaaatatgttatattttattgagatagcatatgatttatagattgtttttagttaagcccaaaagtgattataatagtgttatggattagaagggctactattgagttttaccaaacagatgtgggaagttttttttaatagactattttttgagcagttttaggtttgcggcaaaatggagcaaaggtacagagatttcccatgtaccttctgcccccacacaggcacagtctccccaactatcaacatccccatcagagtggtacatttgttacaattgatgaacctacactgacacatcactatcacgccaagtctgtagctgacactagggttcactcctagagtactatagtctatgggtttgaacaaatatgtgatgatgtgtatctatcattataacatcatacagagtaatttgagtgctctaaaaatcctctgtgctccacctattcattcctccctccccacaaaccctggcaaccaatgatctttttactgtttccacagacatagaaatgcacatagaaatacatagatatgtgtctccatagacatgcattgatgttgatggcagctttattcctaattgccaaaacttggaagcaaccaagatgtccttcagtaggagaatggataagcaaactgtggtacatccagacagtggaataattattcagcatggaaaggaaattagatatcatcatggaaagacatggtgtaaacataaatgcctgttactgagtaaaagaagtcagtctgaaaagtctacatactgtatgattctaactatatggcattctgggaaagatagaaccatggagatggaaagatatcttaacaaggtctttcttttgttattttgtcactggagagtaaagtagtaaagaaaaatggaatacatatctgttgaaaattggccaatgcagttcctgcagcatttacaaaatttcacttttattgtagtaaaaagttcagcctttgcattgtaatttgcatgtttcggagtcagtagttgggttactaaatttgaagcccagtgaatgatgcggggttgcagacattttttatgctgaaatttaatagatgcacgaaaggcatggatttctcatgagttaatttttcttttttgtttatagtatgggtataagtgcgtaagataaagacattcaaggtttattttattttctgcctacnatgaagttctggtcgcccatgtatttacatgcctttctatgaaaacatttttatgaataacacaagattgggaagaactaactggcagttgctatgaaagagccagatccatttactcaggatagggctgactcacaaaggagtggggtatccactaggataaaaatgcagctttttcctcatctcagcaagaggccattaccgacgagctcacccattagcatccgctgttgtctctacttgggaagatcacattgtcactctagtggagtcaggtaagagccatttccaggaaatactcattatctattctcttagttatacagtcatagttttacataattaccactgaaagactattttcctgttgtctgctaatgtgattacattacattaaccaaccttggataatacttttaaatgtattacttgtggagttcaagatttgagcaaaatccctcaaatgtaatgctctgcaaagttatactgcagttcctttagatggtaattacgtttaataccagacggtattgtgtaatctaacttttctataatgagtgaaaataatttgaagactcataggcctctccctttcattacagaatgtataggctttttccacacccttgttctgggacattaagaaagaagacatggagacaaagaagaaaagaaccggggaccccctgctactgtgtctataaacaactagtggtgtttaaaggctaataaagctccaaagatctctcttataaccataatctttgggccaattttaaaatattttagatggagaaatgtcaagaaaaatagagacaatttcaagacatgattctctttccccaacgtccataaaagattatatatatatatatatatatnnnnggctgtcatataaggagcaattgaaattccacagcacaaaggcaatgcagggggaaaaaaaaggatcaatctttcatattattttttctcaaaggcttggcaagaaaccaggggtaatatttttgcaactgggaatgtgtgtttgctgaagaatattcccacatttgaatcatcctcaggggtcctcatcttgttttactttcaaaggtagacagggcataacattaaccccaactgggaaaaggaaaatgcctcactgtaattctcctccagggaacaggaataaagggattacatatgcctgcataatgaagaatttcctcacccagacaagacaaaagacttcattatttttcaccagacacttgaagtcttgtgcctcccagttgcagaagctgaggaggcaggccagttttttatatgaagccactagatggagttcctaagaaactggccttctagtacttgccattgtgaaaagatctagggaagatgttaaaaaccttggtacctgacactacccagtctttcctctaagaaaagtgagactgtccctgaagatcacagtattcttccagtaaaattagccttgcctttactctgggaagcaaataaatgagcattttctttctgaaaatgcacctcctttgtccttacatgagaccaaaaggcatctcaatactaatgaaaaatgtaaagtggcaaaatataacaaaatggaatattcaagttcttctccttctgggtaggtctgttacttcgtaactttaaggatgtatgcatctcatttcttatataaaatattggaagtctcaaattttgtagttactcactatttcggcttgcaagtattagattcctgtaagaggaggtatatggagactatgttcccagcaacagatctgatctccagttaaagaaattgaactaagttcacctgctacagcctgctctgctccctcttggctttcaggcttccctaatagctttcacatacaacatagagaaagangtatgaagtttttagtcgtaagaagcaatgtcttttttttttcattactgtctcttctagtgtaggtatgcagcccattattcttgagtggcatcatattctaacttgagataaaattaagattatttaacttgcccctttaaaagtatattttaaacacatgctgttttgtccgtgattattttcatgcttcaaaagcagggagtgatcagaaaagaaaatatttgatcttcacggaatttcaaggttcattcattccacaaatatttattgagcacctactgtggataagagctgagaaataatcatgaccaattttaaagaaaaccctactctcttagagctctttggagcatggcatatgtgacgaactaaaagaacaaagaaaatggcatggagagaacgaggttcctcctggtaaagatgagaccagaaagggaggttagagggacactaaacggggctgtgcaagccatgctgctcgttaggcttagtctttttaaggcagtgataataaccgcattcgtgatctattgctgcaggacaaattactccaaaacttaagtgatttaaacaacacgtttttcatctcacagttttagtcaggaatctgggcatcccttagtcatgtctctctccaagctgcatgcatttggacttgaatgggaagagccccactcaatcgcatggttgttagtggattcagttcctcgtgagctgtgatatgaaggcctcagttccatccggtttgctggattcctccccaagtggaactctccgtagagcacctcacaatatgcagcttgcttcaccagggcaagcaagcaagatgggaagagcaagtcagcgagagagagagagagagagagagagagaatgaatatgctatgtagagtgttataacatccaccatgacgcgggaaaagatacaacaggaaataatagcaactaaaattcaaagaaagcgttgatgtttccaaagcactgtcacattcataatcatctttacttcttccagctccctgtgaagtcagaaggaaaggcattattgttctctgcattttacatatgggaaatggaggcttagagaaagcacatcacctgtctaaggtcccacagcttataaaagctgaagtcagaagtgtgtgcttttaaagcttatgttttctcaactcatcttctccattaggcttccagacaaaggcagaactttccatcagcagctgcactgcagggtgcacttctttgaaccatgagaggctagcaagttagggatggggtcacacagtccccacgcacctgccgctaccctccctactcacccctccccaccttttcacttactcattctgttcatcaggcagcatgagggccagaaactgaacttcctaggaatgggaggctgagtgataaagtggtccctaatcaactcacagcctagtaaggggaataactaatgataacaaaatgccatgagcgttaaacaagcttggttaataacatactgtaggaacatggagtataaaacaagtctttttttgtgctttcacataggtgaaatattcatttctcatttcatgttacttttcagagcccatttatatcagtctgtcattatactgtgcatgttaaaacactatttggtatttatttcatggtttgccttactgtatatgcttatctatagtaacattgntattccctttaaaaaataataaagcaaacttttaccatgcttttgtatgaacctgggcacattggattagagtatatgtaacctcttagcaggtctgattgtgtccaactgaagttcatagctttctgccaacactgttattttccaaaatagcaaaatttcttacatgaccaacaaatgtgacaatggcttaggctgtacaaggctgtttcattatgcagaaacgtcctatcatgggaatctttctatacctgtctcaatntccaggtgagctctaggccaaagggtcataaaacttccttcccattgccttgctcctgcatccaccgtccaacctcaggtttataacttccatctgcggtccttccctcattcaccaggaacctgtgtgtggccagtggtctccttgtataatggaataagatttttcagaatgttctcgttagacatatttatatatctgtagaaaagttgcttcatgctgtttagtaaatgaaaaatagtgattatttagggtttgtttccaagaatacatttacattcagtgaatttgtatgatttttcatagatattcaccataattatcttcatccacattaaaagcatttttaaggacttaaatatgacatatttagtaagttcttatataatgtattactatataataagttcttatatgatgtattatataagttcttctgtaatatatgtgttatatttaaatacttaaaaatgcttctattatacatgtatcataagagctgctattttaagtgcgctaagcacatacaaatctttttctcaaagatcttaattctcagacagatggtaatcatattcaaagccattcaagaaatgcattttaagaaacataaatgtgaaaataatatgtaaaaaatggcagaattgtgtgttcaggtgttgagcaaatagagtatcattagatttgcatatcataaaatcattgcattttcaacctgtgagctatctttcagagcaaggctttctcttcattttaccaatgaggaagttaggcccaaagatcactcaactacttcataacagtgtaacttctggcacacaggtcttttaatcccttgtgttattttcttgccacttatcaactatctcaagggaattcctgtcattcccagttggagaaaattttctggagaataatttttttttctcaccaaaatcattcacaccacaggtgttaggccacccaattgtcttcactcattagcaanatcagaaattaacacaagtccagttattgtccaaaaagaacatgtttttaaaagtcttcgtggtttaaacaagtggtaactggttaccagtaagctgcaagcttcagattgaggtttctgtaggtaataattccaaaaactgtgtgctttttttttttttttgaactgtggttttaaaaacaacacataacatttaccatctaaaccatttctaagtgtacagttcagtcgtgttaagtatgtttacattgttgtgcataattccagctttagttacttctctttatgactcatttttgcttgtaagcttatgttttcctatcttcctaaagttctacaaaatctccttggaacatatatttaatcattccaggagcctgagtggacaaatccttcccaaagggggtttattagaaaccttgtttaaattcaaaaccgacatcatgattgcctgtgtgtattaatctgttttcatgctgctagtaaagacatacctgagactgggcaatttacgaaagaaagaggtttagtgaacttacagttccacgtggctggagaggcctcacaatcatagcagacggtgaaaggcacgtcttatatggcagcagacaagagaagagagcttgtacagggaaactcccctttttaaaaccatcagatcttatgagactaattcacaatcacgagaaaagcacaagaaagacctgcccccatgattcaattacctcccaccgggtccctcccacgacacatgggaattcaagatgagatttgggtggggacaaagccaaaccatatcactgtgggtaggctggccttctgcttatggtgtcctgatagaagctctaccattatttttggaaaggagggaggaggcagaaggaggaggggcaagaggggagctttctgtttattttccagcccttcttcatagctgtgatacttctgctttgtaagtgttcttgacgcagagcgtaaagttcattttcctttataattatacgttatttgctgtattttgtgttttcattnnggtgttgnccntttaaaataataaaagtaatgagcacttatgtgttattcatctcaagtgtacaaaccatgcatttatcttgggctcctttttttttcctagagcttgcacccgacagcgcatgctgagcggatcatttgaggggcagcccgcaaaggagaggtcaatcctcagcgtgcagcatcatatccgccaagagcgcaggtaaatacttgggaagacagggcttcagagactctaaaaaaggggggttttacaggaaatggtcgctgtacacagtcacggctgtcagccctggctgccagacaggcatggctgaggaatcgcgttctatttgctgtttgttacaataatgaaacacctaatcagatccaaagattcaaaaaaatctctacttcacaatccttttccttagagatggccaaattctcacctttaagatcagaaggaaaaattctgaattttgttagatgggatcaaagacatttggagagagtataagcttctgctatgaaaagaaatcataggcatttgtggcgttgtagcagaaaaagtgttttattttgattttatcatggtcactgcttgaaaggccagtttgatcttgagatgtattaaccagtgcctgtggttgttttaggtcactaagacatggatcaaacagccagaggatcagcaggggaaaatctcttgaaactttgactcaagatgtaagttttaagcaatgctttggatttcagaaacgtgattatgaaaatgtatttaaatgactgcttagacaatgcccagtgtctttctgctgcattcccatgtctccttaagacttcataccagagggaatcaggaaactcaggatatgaattatgtagccaagtatcagaagtaactgttaacagcagaaaactccagtcatcccacacaaagcacatgtcttagttaaaatagtaacacaaaagtgtcattatcagttggatttgacttgaaattaaagacattttaagcattgtatataggattgatttgcatcattgactttgaatattcactgagaatcgaatattctagaatctagatcaagggtctataatttcagggctttatatattaggaagtgaaattttatttctagaagaattttcctgtcaaggactacctagtatttcccaggaaagctttttgcttttagactctaagtcatggatttatttactggctctataaatagttatcaagtgtttgctctgtgctgggttctgttttaaggaacgaggagcagtggggtaataagatagactcagtgccctcctggagcttaaattttagaataggagacagataataagttcataaaaatataatggaatttctggagtgatttctttttattttccatcatctcttcattactgtgacagatttgctttataatgtaaagtaaaatgaagtcatgtaaataaatcaaaggtggggatttgggggcagggaattttagatacgatggttatcgtagacccatctgcattatcccggagacccaaatgaaatgaaggagacatgaaaaaaatgcagagaagccaagcgttatatggcccagcagctataccatgcaaagattacaatgaaaggtttgggaaatgcatattaaataaccgtaattatgaagccctggtaaaaagacaggcgttaggttgaatctggatgaactggcttaaaaagtaatcttgaatgttcagagccaaggatggcacttgagaaaggagggctagcaagagaaagttagcaaagccttggggttatttcgtttttctctttttcaccacttatctcttatttcttgagcttttggagtgcttgcatactacatctgctttcattctgtagaaactgctgttttagtccaagtacatggtttttattaagtgttccgggcaaaatcaagagcgagaagttggcaatgagaacacctgatatcaccaataccctatagacatggttnctagttggcacagagnctctgtttaccaccatgatttttaatatccatagctctatctgattggctaccatattgttgacatagaattgccatcaaatccaacatatggcctgcgcaacttagaactgcagagtcagccatattgcacggagaacattttcaggtgaaaagtcaaagaggcaaccctcattcaaagcgccttcaatatgtttcaaccgaaatgtacaacctacttaagaatcaaatcagaacatgtctttgaaaataatgtgtttaaacatagacagcaaaggccctatttgaatagtctcagtgccggagcttacttcttcnnagagacttcctaccctatttttcatgtagaagtatagtaaaatccatctcccaccttctccatagcaaaatgccatggggttctgatcatttccaataaattcttataatgtcttttctcccacaaaggggagatttaaattagagaattttaactttttcacttattcggaatgatagcangacacagataattaagtctaaggagtgctttctgtaaaatctactgagttactggaactctggccttgcaccttccatggggtccatctaaagggctgtgaagggcctgcatggcctgctcacctctcttagctttacccacactggcatcctggctattccacatgtagacacgccaggcctgctcctagctctggaagaacacatttctgtagtcctaccacagcctgcaccctcacctccttctagtctttcctcaaatagcaccttcatattgaggtctaccctgacctgttcatttaaaatcataatcttctagcctgtctgtgtctctctatgcctttcacccattgtgattttttaaaaaatcggatcactaagcatcctcttacatacttattttgaattattttttatctgtctcccactgactagaacatcctatgagaataaggatgttttgtttccacttgccatatcctgcacactagaatagtgcctggcacataataggatttcaatcaatatgtgctgaatgaattactttttagaggaacaaatactcttgctatgaagactgtggccattaaatatgtgcatttaatcgtatacatattttatatactgtaaatttatatatttaatgtaagtatatacatttactttaaattacatatttaatgtaaatatatacctttagtataaatatacataattataaatatatacatttcatatagatatatatttattgtaaatatatacttagtataaatatacatatttaatgtaaatatatacatttagtgtaaatatacatatttgttgtaaatatatacatttagtataaatatacattgtaaaatatacatttaatataaatatataaatatatgtttaacagtaaatatatgtaattaatatttatactttatattgagggagatattagttttaagttttttaactgataatatattttgcattcaaaaccctcctgttttaacacaccccctgttaatttantttcntagcattccaatacagtgagatatagaaatgcacaaagagaagacagtgaaataaagatgattcaggaaaaaaaggagcaagcagagatgaaaaggtacaggttgctgcagtgtcccatggctctcattcgggaatggcaaaatttcccttagcctcattctcccatgaatttctccttcaaggaaagtgcaagaagaggaactcagagagaaccacccatacttcgataagccactgttcattgtcgggcgagaacacaggttcagaaacttttgccgggtggtggtccgagcacgcttcaacgcgtaagtacacttcatttcagccaggtgtttggcctgagccatgcaaacagcaggggtgttgtcatatgaagaagaaggaacgtgtaatcctaatggggtcacaaaataaactgcggactaattaatcatgctgctcctctttgctgaacacccgaccaggtggaaaacaaaggtcgcaagtagttaacatttatagaaaactcgcattgataaaacacaaaacagccattttcgtttgtgcattttaacagtttcctgatacacttatatcggcattattaactccatattttattttggaagtactgagacaaaaggagactaaaggacatggtgaaaggaaggtcattgtccatgtcttccatgaggcttgtagttcctttgtggtaaaaatacctcaatttgttttttggagaataacgtaaagttgattagtgctttcattaccaactgttataagaaggaaacccactgttcactgtctctaccctaccaacatggaagaaatgtgtaagaagccgtaagtagagtggacattgaagacactagttgtggacacctaagccaagtttgacaggcaacaaatggatgaaatggatttttgtcaagcatacctgtatnagtccattttcatgccactgataaagacatacctgagactgggtaatttataaagaaaaagagatttattggattcacagttccaagtggctggggangcctttcaatcatggtggaaggcaaaaggcacatcttacatggcagcagacaagagagggaagtgaaagcagaaacccctcataaaatcatcagatctcatgagacttattcaccaccacaagaacagtatgggggaaccgcccccatgattcaattatctctgactgggtccctcctacaacatgtgggaattatgggagctaccattcaagatgagatttgggtagggacgcagagccaaaccctatcagtacctttaagtaaatattctgaagacattaagagtcatggaggaaatgagaaaactttccatgttcccattatgtagttggagtgtccctgatgccatcgtttaagtaaattgatttctttgttattaattagtagcatatgataatcactatctgttatggactgaattgttccccctccacaaaatccatacggtagagtccaaaccctcagaacctcagaatgtgactgtatttggaaataggaattttaaacaggttaattaaaatgagatcattagggtgggccctaatctaatatgattggtttccttaagagaacatttggacacagacatatacagagagaagaccttgcgaagacacgtagggagaagacggctctccacaagtcaaggagacagccctggaacagggttttccctcacagccctcagaaagaaccaactcagctggcatcttgatcttggatttccagcctccaaaactatgagaaaataaacttatgttgcttgagccatccagtcttaagcaccagtggtactttgttacagcagctcgaggaaactaatacacactattataacatttcagggatggaagagacctcagggaccactcggtgtcatttgtgtgccccctccttcaacagaggaggacatttaagcgtgcagaagttgagggctttgctcagggacccagctactacctggctctctcttgattgccagactattgttctttctgctgttctataaagactttcacaatttaaagcaactgagtcacaattattaaaatcttccatgagttttcaaagtaaaggaagaaaatgccattttggagattgatttgatcagttcttgaagtacgttttttttatcttgaattatcctgttggctgcctgtaccttaacatctcatggttaattctccacttcacttagtgtgatgtaatttcccttttatcagcaaggcagctgatgttgaatacaattgatgaagactctgttgtaattggggacacatacacaaagactgtaagcaatctcattgacagtgctcacaggcttcctgaaagtagaaacaacgactctcccttccaagaaattctaaataaatcattctaccacatcacagttaaatgcagggggaagaagtctatttaaattatggagtatttaattaaataatcttgaataactaacattcttgaactattaaggcaaagatttcaaatgagtgtttttaaatgaaagtcaaaacttaattttgtactacccatgtcagaaaattctgaacaaagtcacaaagtctaaaatcaagtattttgagaaatataaaactttattttcataagcatcaactgcaaattctttggattttttctttcatcaatgttctctgacctccagagttacctacagttaacatattttataatggacattgtagagaggctgtaaaagagattttaggtagatgagatgtttgcaaatatgatttttagcaagttaaaacatgcttgctttgttgcagaattttatatatgtgttctcttacagatctaaaacagaccctgtcacaggagctgtgaaaaatacaaagtaccatcaactttagtaagcatcaccaacttcattttccatgcacatttaaagttttcatgtactattttatgctgcatacaacgattaaaattctcttttattttcttgctatatagtgctatgaaatagatgattggggaatgatgtttttaaaataccagtcatgttgatagagccacagaagccatcatctctaactcttagaagcctttcctggaacctttatgtttctcaattttgtttggtctttggcagtgatttgctgggattggtcacttacctggactgggtcatgatcatcgtaaccatctgctcttgcatttccatgatgtttgagtccccgtttcgaagagtcatgcatgcacctactttgcaggtaccgcttaccgctgcaaaatctcagcttacaactaggaggtaaaatactgacctgctgtttttccgttcattgtaaccaaactgccttttgcccgtttcagattgctgagtatgtgtttgtgatattcatgagcattgagcttaatctgaagattatggcagatggcttatttttcactccaactgctgtcatcagggacttcggtggagtaatggacatatttatatatcttgtaagtctttgcttattgcctaaatgaaaaatgtaatggttttgtaaaatctcctgcatttcttggtataagcgcaagaggtatgagttacagaggcttagattccctaaccgaattcttaaatggactctagagtagttcattatatgcaaataagggccatgcatgtaaaatctaaattaagagcatcataaatatctatggatagttatattgttcaaatacactaatcacaattttatgtatgaatgagacatacattaataaggtctaacatggcaaatctagtttgcatcccacaaaccttatggcttctatactaaaaaaaaaaagtttgcctcatataaaccatgttattgattatgagccccttattgctgaaagcaaactaaagtgattgtgattgtatctctctcctgattccattgcataaaatacctggctatagcagttgcccattcaactgtctagtaaaactcgctggaaaaccatagaaatgggctcagagaattttttatttttcctgtatgtccaaggaggcatgaaatcaatgtctttgcagagacaacaaggagaattgtgatcattaacaagtagcatgttgcaggtcattaataaaatagcagtaaccttgataaaaggaattccagccagatcgacatcagtggagcctccagagtagtaagttaaaagaagaggcagaacgcactggtgtacacacacacacacacacggtgtggacacccccatttctttcctgcatacacatgatgactttcctttgaggtacctagcaagtctagatgaggtttgttgtttaaagttaacagatggcacgtacaaccagtgagtggaatctctgctaaatgaaaagttgtggccagtcggctgtcagaaaatgtaaatagtaggggatgaattgtataaattccagagatcatttgttcccctttaggtgagcttgatatttctttgttggatgcctcaaaatgtacctgctgaatcgggagctcagcttctaatggtccttcggtgcctgagacctctgcgcatattcaaactggtgccccagatgaggaaagttgttcgagaacttttcagcggcttcaaggaaatttttttggtatgtgagaattatcctttgattccagttcacagtagtccacctctaatgcagctaatgagggaaaagtgagtcttgcttattgaggtcaattcaatagttttggttattgccagagggtaatggaggcactaacaaaacacaatgcaccttcaaacactttactcatgggcacttctggcgtatgagtttatttaattcacctaaagagaagcactctgagatttgaaagagttcntatctgctgactctttcggtactaacctcttgactctaattcctactctgggttttagtttcctcccatctgaaaatgaaggataagaagattcaacacngtgtactccacaaatgggattaggaattcatgcttttgggctcctaatctcattctgtgtgttcctgaaataatatagcacagaatttgtctttagatctcaggagntgttaacataaaacaactttaaaaatgcaacatataggtataacccacttcaattaacttcagcattacagtaatgagtgattacaaangatgattgagggggcaaaacgtactttntagctgggcaaacattgatttagggtcttctgaaaacaaggtcagagataaaacaacaagaaagaggctcattgttcacaatctgttacagaaagatctttgccttacaaaacagtgatcaaaagttttttttattattgatgaggtctgcttgtgcatgagaagcatgaattaatttacataggcttgttttgtatcttgctttctagagacttacctgtgatgagaaaacagctgtagttgtagttgacacaaatgtattttaaagatagattgtgatgttactagaggtatatatatcattactgactttttttttctttttttttgagacagaatcttgctctgtcgcccaggctggagtgcaatggcgcaatctcagctcactgcaacttccacctactgggttcaagagattctcatgcctcagcctcccaagtagctgagactacacgcatgtgccaccactcccagttaattttttgtattttagtagacacggggctgcaccatgttacccatgctgcccttgaactcctgacctcaggagatccacccacctcagcctcccagttgctgaatttcttttttaagatgggtttaaaaattcttgttttctttatgcaaagccataaaacagtgactcttcacacagctggaattaaaagaaaatatcacctgactgctaatgtatatgaatcttttgatcaataataaattttagaggagaatttgtgcctttccctaatggtggtcagtgtgattttagtacacttaggtatatgtttaggttattcaaccagtgcagttcacttgttccatattttgccctctgccactcgtctctgctgagaccaaagaagtcagccagcagagggagcatgagttaattaacagatttgctcttcaggactgaattctctcctctcctctatgcatagaacattctcctaacgcacaccttcacagttacaagacagctaagccactgtcccgctcatttgtgctctgctaataaaaatatgaatccaggccgggcacagtggctcacgcctgtaatcccagcactttgagaggcccaggcgggtggattgcctgaggtcaggagttcaagaccagcctgaccaatatgatgaaaccctgtctctactaaaaatacaaaaattagctgggcttggtggcgtgtgcctgtagtcccagctactcgggaggctgaggcagtagaattgcttgaacccaggaggtggaggttgcagtgagctgagattgcaccattgcactccaccctgggggacagagtggcactccatctcaaaaaaaaaaaaaaaaaaagaaaaaaaaataaatatatatatgtccatttgggaaaaaatagacaaaatataattacttaaacatttttttcctttgttccattaaataaaaaagatcaaggtgttgattttaaagtcagaaaagttcaagccatatcaaaaataccttattccattttcttacatgaaattcatgtttccatttaaaaaattcaaagcttgttaattttctccttttgaccttccagtaaggctaattgtgtgacctatggtggagaagttaaaatatatcatcaaaaacccatgatttttatacaatgttatcttagaagtcctatttatgtcactgtttaaaccaaacctaatatttagtctttaacaagagatttaacatttcatcaatttctttcaggtctccattcttttgctgacattaatgctcgtttttgcaagctttggagttcagctttttgctggaaaactggccaagtgcaatgatcccaacattattagaagggtaagatgtatctttctgtcttttgaaataaaaattaagggccaagtgtggtggcatgtgcctacaatcctagcactttggaatgccaaggagggaagattacttgaactcaggaggtcaaggttgcagtgagctatgatctcaccactgtactccaacctgggcaacagagcgagagccctgtccctaaaaagaaaacaaaataataataaggtgtttacactcccttggcaagcatgtggtaccagtggctgacaagcctagtttaagaactccctataaaaccctaagaattctcttcccacaaacccctttttgcttaagcatgtgatatgtctcttctggctagaaaggttaataagatgaaaagcacacctcagagaatgtcactcttccctcctattccagtgtcaacccactgttgacaacttgagygtggtcacccaagattccagctgtttacagctgtaagctttggatactttcccttgaggtcttagctaacttgatccttcctttgttgtcacataagactcatatcagcaaacagccccatgcaaaaatgtccaagaagcagcgtttggttctcaccactttttattgcttccccactctcagctttctccccattagaaangccagtgcctttacattagctctagctgcccagaatctccctctttatgacaaccaacaaggacctccacaaagacccaaacttgaatacttgcactacggcgtttctttctctttcttcagtcattggttataagggaaggctagctgctgtaacaagcccaatatttcggtagcttcacataatgaaatgtatatattgcatcttggatcagttgcaatatatacatttgcaatatatacactatatacagctctcctccaaatgaccactcagggactcacatgccttctatcctatgccgccaccctttgccatgtcttcagagccttcttcaatgagctggagataacagtggagagccatctccaactgagtgggctgtgggagaatctgctccacccacattcctctgtaaagagcagagatactgcaaggggggctggaatttagttgtgtgctccagaaaaataaaagggggacacagtttggttaataattagacaatttttgtaatatcataaagtgtcccattctcttaggattcttatgttacaagtaaattcataaagtaaaccaaccactgctgatgtataaactagaaaagcattttctctttgatcccaataaaatgatgcctcagttcctaaatgtatatcttcaaaaggcctactagacctgaactacattatttatttctagcaaatatggctatctagtatagattttaaatgatacaaattgttctctaattgtgacaaaatacaggtaacatctttgttcagccaaaatacattataaattatattaataatgagaatttgaaacaaaggccagactgggagagacattccaagagctttgatacacctgagaagacagaacaaggaatgtggcctgacggtcatgccgtatcaaattagctgataaagaaagtgacagagacctgacgcattcatccacaagcttgtgtgtggaaagaagagtgatttttgatccacactgaacagaggccagaaagataataacacttcaggatgaagcctgctaggaatcttggatgactcagtatattaacagtggcctggccgggcgcggtggctcacgcctgtaatcccagcactttgggaggccgaggcgggcggatcacgaggtcaggagatcgagaccatcctggctaacaaggtgaaaccccgtctctactaaaaatacaaaaaaaaattagccgggcgtagtggcgggcgcctgtagtcccagctacttgggaggctgaggcaagagaatggcgtgaacccgggaggcggagcttgcagtgagccgagatcccgccactgcactccagcctgggcgacagagcgagactccgtctcanaannaaaaannnnnaaanannnanaannannaaaannnnanaaaaccgggggccctaaaaacgggggggttttactttttccccttatccccccccaaagttaaaaattgggggggaccgccccccccaaaaaaccctaacccccatttttaggggccttttttcctttttccaaaaacaggggggttttgccaaggtttaaatggaaaccctaagggaacctcccctttttaaaagcccccccccccaaccaaaacctttaaaaaaaatttggcccgggaaaacggggtttaaatttttttgtttttcaaaaaccaaaaaaaaaaggccggggccggggccaaaaaccccttaaagaaaggggacccccgttaaattttttttgaaaaacttccccccttttttctaaaaaaaaaaataattccaaaaaaggggggaaaggtaatccccccacttttaaaacccccccctttcctttaaaaaaaaaaaaaat

SEQ ID NO: 3 - DNA, 47841 bp, Homo sapiens
  exon 3895..4001: exon 28
gtttaaaaaaaaggtgaaggcctctnggctttttttccccaaatttttttgggaaaaaaaagatttttttttcccccctccacaagggcccaaaaaataaaaacccttcgggagaaacccgcttgggaattttggggggctccgggaatttaccaagggcccggcccgggggggggggttcccccctttattccccccccttttgggggccaagggggggggtttccagggttgggggattaaaccctttttggttaaaaaggggaaaccccctttttttttaaaaaacaaaaaaaaaataacccggggggtgggggggggcccctttttcccccttccttgggggggtnngncannaaaaagggnntaancccggggggggnggtttncantaanccnnaatnccccccnngccncccanncgggggggaaaannnnanancccnntcncccanannnanannnaaaaaaaaaaaaaaaaaaaaaaaaaaaaaaaaaacagtggcctaagagcatggtgattttacattatccactcatacccagccaaagctgaagattgatggtgacagcagccagcagatgagcatagacacagatttcatgatgcatttatcctgtttcctagagcagtgaggtctgtgcgaggctcagatggaaagcctaaggagactcagccatcatagagagcaccagcaccaagcagaacgattgaaaagaaattggcacggtgagacggggcttgagtctttttgtcttctcaaatccaaacataagaggccgaggccgaggccaaaaacgccatgagtgaaggtgacgtcggctgaagttctgttggagaacttcagcccatcttactaaaagaagagataaatccaaagaaagggggaaagtagatacaccaacatttagatcacacacagttcctgttaaagacaacgaagcttcagttgctgcgcccatagtttccagttgggaagatgtgttttcttgtatgaggctgagcacttcctccctctgctcctttgtttgttgtcttaaaactcaacagatgtcatgagatcaatatagatatagatatggagcctaggactttgtcaccaaaaccctgtaaacaaacgctgaggataccttttgacaaatagtcattaaatgtcagttgtgtccaaggcactgtgataggtgaaatgaaatgtggaagggaaacattaggaggtccttgcttctgttttgtggcattttctgtccaatcagaacgtgtttcatgttcaagcaacagtaatataaagtcagcagttttcaaaagcctatgagaagcagaaataacatgtaatctatgatgtcagcatgggaaattgtgcttccaactgggagtactgtactgggagggcttcagggaggaggtgggatttgagatgagcaggaaaggaccttccaaggagagcaagcagtgaagaaggacacggagcatacaaagaagggtcacagtgtagaggcagggaccgaacaaggaggaaggagcatgggcttcgaaatcacagcttctgagattggaatcctacttcaggagaggtgatggcctggtctggtcacttaatccttctggaacctatttctgccttgaatgcttctcacacaatgatattatgaagattagattaccttgtttatgcgaagtacctaacctgatctcaacagtgagtgttagtttcattcccgcccatgtaggaaaaaactgaagaacaaggccagaaatgtaagttagggagagatcctcaagtgcctgaaatgcctggctaaggggntttggaatttcgttgatgagaagtgagaaaccacaaaaggttggtaagaagaggaatcagacacatcctagtgcccacaaagtacaaagcaccattcctagtgcagtttttgttacccatatcctaatgaggatatccattcctaatgaggaaaacaggtgtagaaagattaaataatttgtccaaggtcacccacctaataagtggagaagcagggaactagtgtccaggcaccctggcttttcaccaagataccagactgcttcatgcaatcaaaggtgggctcaggtgggttcaggaagacaaattggaaaaggcagtgaggccaaatatgtcaagggacctttatacaatacactacaacacagcnctttccaaataatgttctacataaaaaggtatggttatggaaagataatcgcaatgtagttttagctgaaaaaagtgggcgatggaagaatataaactttatgagctcagatacatataggaaaaaagactggaaggatatactccaaaacgnttcattgtgtttgtctgtggcaattgtgggtaagttttcttccttttatttgtccaggctttataaattaaagnatgtgttatttgcattaaaatattcttattttaaagaatcatgctggtaatccagaaagaagtaacgttggcattactaaactcatcgtagtaagaatggagacttgggccaagaaggacagggaaagcccagggaaccctaagcttgatgctgggctnctgctgggatgctggtccagggacagaaaggaggaggccaagaggagacatggcttagggtcaaagaggacgtgaagcagattaaaatggaatttttgttgaaatattcaaacagtgatcaagaattttaccatctacaccttgaacccagaaaagagatttaggttcaaaatatgaccatcatagccaaagaaacaaaaataaccaaagggagaataggaaatgagtcaaaaacttcctatagatgtttaatttttaaaacctttttaacagctttattgagatgtaatccacataccacacaatacacccacttcaagtgttcaattcaatggttttttagcgtattcatagagttgtatacacgacagccacttttagaacatttgtatctccttgaaaagaaaccctatgtctcttaactattacccccatccctttagcctccacctcaagacctaaacaacaactaatattctttttgtctctatagagttcactgttctggacttctatatgaataaaatcataacatatggtcttttgtgacttttttctcaattagcataatattttcaaggatcctccatgttgtagcacgtatcaggacttcatcccttcctatggctgaataatattcacttgtatggttggaccacattttgtttatctgtttgttcaaaaatggacatctggattgtttccaccttttggctattgtgaataatgcttatataaacattttatacaagtttctatagggacatatattttcatctctcttgggtagatacctaggagtggagttgctggctcatatggtaactctgtgttagtggtttgaggacctgctagaatgttttccaaagtggttgcaccattctacatccccaccagtatgtaagggttccagtatgaggattgcgattcctccacatcctcgccaatgcttgttgttatctgactttttggttatggctttctcagtgggtgtgaggtgatatatcattatggttttgatttgcattttccggatggcagatggcacatgatactgagcatcttgtcgtgtgctttttggccatttgcatatcttccttggagaagtgtctactcaggtcctttgctcatttaagtaatattctcagcatcgatttttaactgcctgtctccaaagtctttgttactatttgctgggtagctgtccacatcactgttgttagataaagggcttttgggattccgtgaaagaagaaagagtttcctatcctttatcttttctgagttcattttctgaccaggtttcccctctcctcttgtttttctactttaggaagattgcaatggcatattcagaattaatgtcagtgtgtcaaagaacttaaatttaaaattgaggcctggagagaaaaaacctggattttgggtgccccgtgtttggtaagtatatcataaaaaaatattctgaagaatggtgtgtttgtcantataaggctttaaataagtatcatatggcatgtaactaaaagtagttttgctagagatgttaaaatgatcttaagtcatacagggcgaagcgctagattatattggaagatagaaataaggtaaaaaataagagagcaagtagtgcaatgagttctgtatagcttcatgaaactagagaaatattatctcagtatttttctaatacttcagaaatatgtatctggattcaaatgccttaaaaaattaggcttaacaggattttctgtcaaattaaaactaaacttatcctgagtttatatctttaaaggaggatctgatgtcctcataatagaaccctaaatcataaagctatttccattttaaaacccatttacactgaaggacttccattattgaaacactagagaggaataatcagtgacacagttgattgcttctaactttccctttaatattcctttttctttaaattgagttttcaaagcacttagatcaaagctagaaatacctagtaccttctttcttattttattaatgaccatgagtgaatctctcagtctgtgtcattggtgaactgagtgttctgtcaccttcatttgtcctttaaaattnttatctgttttttaagttttcttttagaaaatgtgtaagaataacaactgagtcacagcacctctgcttttgtcacaattgatgttcttttctaaatgtcaaaactctttaaaaggaacagcatttcacccagctgtaccctagcactcactgttacctctctttggcatttttctcatatgaaattcgcttttatacgtaatccaatagaatgcatattgggtttttattttttaaaatatccaagaactgagaaaaaggattcatttggggtatgtggcttcagcttggagagagaggaatgtgagacatgagaatatcctggccccactgctgactagaaaagccgtccaatttccattgaaataaaaattaaaggagcataaaaattttcttccatgagataaaaattaattatagcataaagttatttatgttataatttattaatttttattaattcaaagataatatcatggaagtaaatgtagtcaggaatagagaacactggggcatttcatttccttcactatagagaaaggtagagggtgcctcccacctctgccaagtgaggtaccccagggggatcacttaacgtcagagatgggcagggcaagaagagagactcttgtggccaggctgctcatcaccatctggagagcagggttatctggtgttcctggagagcccagtagtcactgcctagggccacatcatctgagtcctcttgagccttgcggcaaagggctttgtgctgggaaagggtaaccccctccaaagggtccgtaggtgtgtttccaggagccagacttgagaatcacccagagactgcacacttacaggagaaaaggtgcaatgaggcactcctgggctggagagggggttcaaactcttgaacacctatggccagaatccaattgtatcagatgcacaagacttaaactccattcctccattcctttcaacgacatcccacctcagtcatgtgagaatctcgtggtggcctgaagaggatgagtggaagtgaagaaagtggccaacttttcttcctttcctcaaaccggggtccttgagctgctaaggatgggattgttatatgagttataggatcagttggatgtaaaactgaattactgaaaataaccaaaaagctctgggacctccccaagaattcagacagggagctggaacacacttgaaggtagtgtttggagaaaagtaaacctgccagttgtgccctatagcactcaacctagtccataaaaccagctacatgggatttttaaaatcaaagttaatttaaatgcaatattttatggaaggtattattaggggtttcaaaggcaatgccattctagaagcataagcaaatagccaggcattccttgaacatcatcacaagtgctcttgtccatttctccaggataatgtgacttggattgtagtgaaatagacaccttcgatctaataaatgtcatctgtaaccatgataactttttacactttccttcttaagtaagctaatcaagagttggaagctgaaattttgtaattttatattggctaaattatgattaagaaaataacatatctaggaatacactagaggaatgctttggtaattataattaggacataaatatgcactaattcaggagacagtctagtttcaatattctctcttcagattaataaatgtactttcttctgaatcactcagacttgatgagtcacctcagtaaaaaggattaaacatatcatggattataaatacagtgtgatttattttaagaggaggtctggaatataggaaaatggcatgcatgggagaaaaactaacaaagtattcaggcttataataaatgtatttattgagtgcttgctcttcaccagctagtatgcttgtcactgtggtaaggccctgaataagcgatggcccctaccccttaggagcttaaagtctagtgggaaacagaaaataaaattacaaataatttnatataatatgctaagggctttaatgctgcacagagaagcccctaagcttacctctggctcacccgaaactcatctccatgtaatgttaagctacactgtgatatgttctaggaagaaggtgatcaggttttatagggtcnaaggcccaaacaatttggggtaccaacatctcagaagggcctgtacaaaagaggggtcctaccccttaggtttctttggcttcatcatatacctgcctctgattcctacagaaaggctaagggtacaatggacacacaggagaagcacctaaccccaactatgcagggccaaagagaaggaccagatgagaagacacactggttaccttttgaaggacaaccaataggaactagccaggtgaagggggcagggtcagaggcaggtgaggaggaaagtctaggcataggtttggtattacaagttaaggcaactctatgcccaaagctcaaggcaaaaagcgttggggtgctgtaggaagtacgacgtttctgatttcttttaggagagatggttgtgtaagatacagcatgccgtttttggacaaagtctttttctgcatttccacatccaagggcagacgctcagtcacgtcttcagggtgcccccatgttccccaagggaagagcagctctccctaccttgatcttggggggccttggagcacctcctctgggaggtaaaaaactgacaaaattatgtggtcagatctaatgtcttagttttctgaattggacaggcaagattacaatatttcacaaattaaccagaaaaaatgacaaccttttcaattcaaggaagggtagcaggtaaaaatgacaataatagatgncatttgggatttattgaaggactcngtagttacaggtaaagtttcaaactttgcaactttgctagcatgaagaaagcataagaaaacactttacatgattaaaactgaaaaagacttatttttttaatgtgataaattcttccaactattgaagtctttgatgctacattttgttttacttggtttagttagaatatttttcacatattctaacttctagaagatctagaacataacagtagtttcagtggctgcaaattcagctaagaaagcacttctacaaaagaaaatgagcaggtaaggaagtccaacaccggctctgagaaccggttcacttgcttgtaaaaaatccttattcaagcaaactgtggcatctgtagaatccttataacttctgtctactgtaaaatatatgaaatttagtggaaacatcccaaaggaaagattgttggaagcatccaaaagaatatatttgtactttcatacataaaaaaatataaagcagaattccaaaatatgcatttctgagtggggcttttgcactatattaaagaccgtctccactaatcagccctaagcccttaagaacaaatcagtttcagtttaatgttttcttcttatatcctcatctatttaatattcaaaaaacttatgaaggcacatcttgtgattgtctctatagtttaaaaaacataaaagatggtagaaaagtacagaaccagattttccctagagcgttgcctgtcctgagggttaaattttgagaaatgagctagcagnngggnaaaaaatgatcaaaatataatttaaataggctccaaggggcctactctagtcagtggtccggagctcagttcttcattaaattcaagaaatatagtccatgattttccagtgtttggttatcaaaatcttaattatttaaaattagcctctaataagaaggaaagagggggaaaagccactgaaagcttcttctattaacaacaaaaatgttagctaagaaccataacaattaaagaagatgcaatctagtaaaaaggatcacttttataattgggaagagaaaagtataaccatgttaaaagaacaagaaatgagacatccctggcccccacagaagtgggagctaaacagtgggtacacctcgacataaagttggaaactctggacagcggggacttcaaatgggaggaggaaggaaggaggtcataggttggaaaaattacctgttggattctgcgtgcactatctgagtaatgggttcattagaagctcaaaccccagcattatgcaatacacccatgtaacaagcctgcacgtgtaccccccaaatctataagaaaacagaaaatgaataagccaatgagtttcaggaatcaggaatcagcggtggaggtagaaagcataccacccataaaggggaccagactaagagtccactgagaagactcagagttaataagaaaaaatccaactggaagagagtaatttggaaaaatacatactacaagcaaacacctcttgccgtctcagtgacctctaaatgcatcactactttccagccctgtgttcaccatgatgacacaaagtgaaataaagtgactaaattctactctacactttctatactaacacaggcaattgtaatttatgcctttcgaaagattttcagttacaaaactatttcaaaatccacatccaaaatccaatgcttctttgtgcaaatcgataaaatcatctgtccagtcctcagggtaatcaggacaaggtagaaatgttgcagaacctctcaagctagaactagagctgttttacattgagatcaccccatcattacctaatgaacacaatcaattataacacattgatgcattgttatgatttagcaaacgaatgccatatagcgtaaaaatgtgtgtgtatacatatacacacatatgtatgtgtgtgtgcacgcacacgtgtgtgtgtatatatgtgtgtgtatatacacacacacacacatatcccacactcatgactggaaagggctacagatatttgccttcgggtgctgcttattggaatttcacagaacattcctgtattggaatcgcagggcgaatcctcggaactttaatttcgacaatgtgggaaacgctatgctggcgttgtttgaagttctctccttgaaaggctgggtggaagtgagagatgttattattcatcgtgtggggccggtaagcgagcacatcgaatcctttgaacacttcaagagcagctttcatttgttttgatgaataaccatattttcctttatgcagatccatggaatctatattcatgtttttgtattcctgggttgcatgattggactgaccctttttgttggagtagttattgctaatttcaatgaaaacaaggtaaggatttttggtttggataccacagggtttagccttttctagccactcactttgagctatttgtttcttttcacacgcggcgtgaagccgctttaaactgtaatgtgactggtgtaaataatctctataactgttttacaagctccctcattgctgcttttcagacacattatttaagagtacagttctcttgctaaatttacccttttggtaacagtgtcattaattcaccgacaactgagaggcgcgtctcctattgttcaaattgaaaagaaagatgggcagcaagttattaaaatacaatagatgaaagtgattcgctaattacaggtaatattactttcactaaatgtaaattgtatgcttctgtgtaactcagtaggtgaactcaggattttgatgggggtgaggggagtctggtagaaatttcaaaatcctaattcacagaaaaaaagattttgtggcagggcatggtggctcacgcctgtaatcccagcactttgggaggccaaggcgggtggatcacttgaggtcaggagttcaagaccagcctggccaacatagtgaaaccctgtctctactaaaaatacaaaaattagccgggcgtggtggcgcatgcctgtaatcccagctactgggttggctgaggcatgagaatcgtttgaacctagaaggcagaggttgcagtgagccgagatcacaccactgcatccagcctgagcaacagagcgagactcaaaaaaaanaaagaaaaaaaagaaaaaaagattttctaatttcatatgaacttgacatactttgtgaactcgggaagcctgttgagtttgtaacagctctaatgattagaacaaaggctcacaaatgggaaaatnacaatgtagtttcctatcaaagtgtgagtttggggaagaacattctattagcactccttggagaggggcttgctcatctaggcacttgagctatgccctctattgtttgagacgcagtctttggaatcatagtaagggaagtgcatgcagtttttgagcacacactgtgtactaagattttacatattcaatcaattctcacaactctcaaatgcacggttgcagaaaaagcaatgactagtctggggcttataaggtgtganaaacagggccagaattcagaattggtgcctattaccgcaggctctgtgattcttcgaaggcatgagtagaataaagtcaaccttcactctgcattaagttgaagctagatctgcctgtaaagcaccgtgctgctcgatgttattcatggctaggactgattcccatgcgttcacatcagagctcttcagtttgtgttgctacacatgggactgtatctgtcattcactgtcctcaggctcaacgtgagtggtaccagttagccagtcaattgacacctttgatgtgtggctaatgagcatgtcatttcattcaggctttcaaagggtgagatttaaaaattcctcttttgggccccgactctacttggaccctcagaaacatgggatgttagagacagaaagaacagtagtgatcatgtcacccagccccttattttactcctatcacattcattaacgagatgctttgagccctacagtgttgagtttaataccaagcagaaagaggaagaaggatctttttagatgcctttgagccatgtaatgcagcagacactgagaattctagaatataagcagaagctaatcagtcaattaacaggagagtttaggcactgcttgcccagaacaccgttaagaagccgtctgagcagacacctgatttcaggaagattagaatgtatcacagctcacaattcagcaacatacatagaacaacataaaatgttcagtaactggaaataaatgtataattaattgcaacacttgagcttagagaagtggctgaggttagcagaataaccagggaggtctccatccagcactgtatttatgttctgcaggtaggaaagagtaacgtggcctgtttgggggatctggggcgtatgttctatcagctcaaatgggcacttaaagaaaggagaaattgacaagaaattatattaccaagctttacaaacttgatttgttaattggagatatcaattgctaaattatgtgagccttttagaaaagtttctttctctgcaagntttttttttttttttagaaagcagctcatgagctgccatctaacagcatttatacattattcaatcattcaacagatgtatttgagcatctactaggtgccagaggctgctcttgacactgaggacctagtgaactaacctaaacactaaacacagaaaagttatttgccctcatggagcacacatcttaatagtcatacccaaaggcacataggtaaatttccaaatgtagagacaacagagatagaaaatgagggaatctttagggtacaattcagaatcatgcaaaaatatgtcttcttttgaaaatggaaacctctttaaagagttccaagtctatattagtaatatagatttttttctgatttctctactatgtaagacaatgtgtttgatttattaacctaacttgataataataaactttcagaggtttttatatattaatgatcagatgatatctccatgcctattattcttttagatcctttcagagttcaactttccaaaattttttttatgtataacaaagtaattacattagtgtctatggtggccagagaacaagtgtttgcttataattagtgagaaaagtggtttagcaatgtgattatcagaaaagaacaattttaaacaaacactattattgggaataaatgatgtactaaagaatgacaaaataatcaccagaaagatatagaacttccaaatcccaaaatgtacaaagcaaaaattaaacaagttgtaaaaagaagctagaaaatccaaaatcatagtgaaagatattaacatatatctcaaaaaatctcataattcagagnagaataaaaggaaatatgagatttgatacaataaacacaatcaagtgaatgtataaggaaaactctaattacaacaatttgagaatgtgcattattttcaagcacaaaatgtntctcccagaaggccataattacccaagaattgataatgtaagaatgttttctgaccataatgaaaacaaaaacaaaaaaaactctacaatttggaaatattaagacaatactataaataatttatgagttgaagaaattataatagaaaatcacaaaatatttacagacaaaacattttatagcgtcatatacaaaaactaatgaggtataggtaaatcagtaattacaaataaatgtatcattttaagtgtatatattaaagaagaatcacaagttaaatattaatatgtcaaatatccaaatcaataaattataataaagtaaaatcaaataaagtaggggggaagaaagtaacaaagattaggacacaaattaatgatacagaaagtaaggaaacttgtatagcttctataatattcaagaataacaaaagctgggttttggttttttttaaatcaagtcaataaacctctaggaaggttgaacaaaaataaaaacatgaaacaagaaagtaagctacaatcattaaaagagtgtcagtattgttataggtatagaaaattagacttacagaaaaaactagaaagacccagcagggacccatgtatgtagaaaacagaagcagctcaacaaattagtgcagagggtggtgagaccaacaaggagtggtgttgggagtattgattgtattgatgtaaagagctatgctgagatctctacctcactgcatactcaaaaatgaattccagatgaattaaagacttcaatatgaaaagcaaaatttaaaccatttttaaagcatgtcagcatttattttttatttgaatttcagataagcagcaaataatgtttttgcctttattatttttaatcagcgcataattgtacatatttatcnggtacagtgtgatatttcaattcatgtattataatgtgtaatgatcaaatcaggataattagcatatctatcacctcaaacatttatcactgttttgtgttgggaacattcataatcctctcttctaggtatttaaaaataaacaattaattgttgtcaattagattcaccatatattaccatggaacactaaaatttannttcctccattctagctgtattttcatatccattaatcaacatttgactatccctattctagttcccctccccacccctagtaagcatttgttttttgccctggagtagttatggatttcatcccattagccaccaagtgaaatctataaaggaaaaaaattataaactcgattatatttaaattaaaattttctgaatgacaaaagacactctaagcaaaataaaaatacaagccaagactaagagaaaatatgtacaacttacataattgataaaagattagtatccagcatgtataaagaatctctacaaaccattaaaaagtaaatatcaaaatacaaaaatgagcaaaggatatgaatgggcaattcacataggaagatacaagcatcaccatttacacacatgcaaatgtttcatctcgttaatattaaaacaacattagatacattttcacacctatcaaaatggtcagattacaatgtctggcaatatcaagtattgcaagaatattaggatatggtaacacctgtgtattgctggtaggaaatttgttttggttnttgctctttggaaagcaagttggcaacatctggtaaatgtaaagatgcttaaattcctagaccccatcaattcttctcctagagatcaacaatggagaaaccctcatagatatttactgtgagacagtacatggatgcttattgcagcattggttatagtccataaatcaaaaatgaataacttcggatattcgtacagtggatggtgtttgtgagcagttaaacaaattagatttagtaatctagttgtatggaaagtatactgttcaatgcagatataaagaaacagcnnnnataatcacatgttggagtccatttctataaatattaaaaaattcagaacaaattcttacgtattgattcatgcgtaagcacaaaagtctgaaaacatagtaaggaggatgcacgcctgaagggtagtggttagctctgggtaaggaaagggagagtggggtttttgtctagtttttaaaattacacttcagcttttatgataaaatataatgcagtgcagtgcaatacaatataatataattttaatcatatgcattttaatcaagtactctattatacagaatatggaattccatttaggcagttatcacagcactactttcttaaaatgcaaactctatgactttgtatctctcaaaaacagttttgcaattgcacatattagacattttttaaagaaagacaacatacccttaactggaagccacagcattgacaagctataattttgctgctatgaaatgaaaggaaactaactactattagtgagtatccttttttgaaaatgtaattatgtagaaactcctctttacattttcccaaacctgaatattacacatctccaaagatacaagtcgaaggctgctgaattctctctgaaacacctggtcatcattttgggaactcctcccaagaccaactttgagcattctgaccatgtttctctcaccaccacggttggaagaacatggtcagtgcaagccctcacatcagtcctgtttgtctcggcaccccatctgccatgttcttggtgcaagcacatgacaaaaggccttttcctgctttcttttgtctgttaaggggacggctttgctgaccgtcgatcagagaagatgggaagacctgaagagccgactgaagatcgcacagcctcttcatcttccgcctcgcccgggtacccaaaatgctctgattttattcatgttgggcactagtgtgcacgattccccaggagtctcagatttaaattaaaataataatcgtgaggccgctgggaggttgtgcataaggtaatacagaaacaggctccaaaccacagatctctccataaggctattaaaatcaaccacccaaaggctgtgcttcagtgctctcagactaagccttaatgaaaaaatgaatttttaaataaaatatatttgctcttgccttttaaaaatataagtgaaatgtattaaactgacagaaattgaatgatgcacttctccttccaatatagacggcatcatgcactgtcgttttgaatattcagagccagccattgctgcaaaaacacctcacaatagacggaactgtttggggagaaaaatgtaatgggttttaagataagcatttaagaaaataaacgacaagaaagtaaatgtgacctgtgtctaaaatgcccttgcccaaacctttcttttctagataatgatggttttagagctaaaatgtatgacataacccagcatccattttttaagaggacaatcgcattactcgtcctggcccagtcggtgttgctctctgtcaaggtacttcgtttcaccctaagactccgggaatgaattttgtattcactgctacatccgagagtatcacaagctgctatatgcattcacggttatgttcttgggtgtaaaaattaagattcactgtcaatggcaaagtgggcagaagaggggcaagcctgtatgtgtccgatggacgtgggcaacgactcgtgtgctgtttctgttgcagtgggacgtcgaggacccggtgaccgtacctttggcaacaatgtcagttgttttcaccttcatctttgttctggaggtactgtaatgagtcagctagctctgggggaatctgtgcacagttttaaagtaaatcagcctccctctttgatgccacttactctcctctctgtgttccattgcctttgaagtttgcccttaggatatttcctacttaagcggttctgcttaactcgaacaaagacatcactttatccatcagatttttctaactacagaaatgctgctgtcgattgttgtgtctgaacattggcctgtctagttatgcgagttctgatatttagagggaatggtgcaagccactataattcattgactcaaattgcaatagagacgcaaancaagttggcccagacaaggttattttaacatgtgattctcataatattgctttccagggcagtcttcctgaaatacaagcagattcttattttgccctacactggttgatgtttcccaaaatctatggccttttgcttaaggaagaaaaatgtcccatgatcagaactagtcataaaacgaagaggaaagagtgtagagaacacagatttgtaagctacttagcttcaaaaataaaatcatatatcttggtaataaactcttatttttagttactacatgtttacaattattctttaaatagatagtataaccgagcatgctggctcactcctgtaatcccagaactttgggaagccgacgtgggtggatcacctgaggccaggagtttgagaccagcctggccaacgtggtgaaaccccatctctactaaaaatacaaaaattagccaggagtggttgcacgtgcctataatctcagctactcgggaggctgaggcaggagaattgcttgaacccgggaggcagaggctacagtgagccaagatcatgccactgcactccagcctgggtgacagagcgagactcagtctcaaaaaataataataaataaatagacagacagacagatagatagtgtaggcacgtggcctaaaaggaaacctcaaagggcattaaagtgtttgtctcattttcaatggaaaagatactgtgtccacatcaaataatttaaaattttcttcatttgaaaaaaatacatacacataatgcatctcaagttaatacatttaaatatgtactaaacactctctctgtttattttcttctttgctttttctccaggttaccatgaagatcatagcaatgtcgcctgctggcttctggcaaagcagaagaaaccgatacgatctcctggtgacgtcgcttggcgttgtatgggtggtgcttcactttgccctcctggtaagcatagaagtcaagttgatttcagctgattatttcagtttggttcagttctgctcagtcctgtttccagtctcattttactccacatcaacctgctctgaggtgttagatggacctccctaaagcccagctttgactgtgatgggctctcagacatgcttgtgtctacatgtacacacccccacactcttcacacaggcaaaacctggcccctcaaagacctagacgatgcaagctagaaagagaaggaccttcaagatcttctggtttagcccctcattttaccaaaggggaaaacaagaaagagagaggaagactgcaacagagactgagaactgtccaaattcacacaagctagtatcagaagactatagtcctgttttcctgttttnctcagctgaaatatagcatttgttccactttgttataatcagtatttcacaatgtgattctcaagtgcttccactgtttcattttctataagacatttagcctccacttctctaaagcatatgattctccatctctatatctgtgttctcactattttacctaaagtgccttcaccctccattcagtctgagatcccactcaaaatctgcagctatgggtntactgaactgtaatctagggccatattgtccaatgaaaataaaatgtggccacagatatcacttaacattttctagaagccacaataaaaaagtagtngtttagaaaatataactattaaattgttaataattattaannnnataaataataattaattatttaattangtaatatattttattatttaaaatatgtattaattaaaagatgcattatgtgtatgtgtatgtatatatttttattatttatatagttatatattatatattaatatatttaattaaataattaatttaattnattatataattaanttaataattatttaattatataattatttggttataattatttatgtataattattatttatatacttataactattatgtatgtaattatacttatgtaattataattatttatataatttaattattaacaatttaataattatattttctaaactatatccaaagtattattattttcatgtgtaagcaatataaaattattgagataagtagtatccttgtatctgtgtgtgaagtctttggctgagtatgtatcttacatttacagcatatcttcatttaaattgccacattttaagtgctcagtaaccacatgtggttccatgtggctgctggttaccatgttggatagcacagctctggaagatatgggtcctagtctaggttccaccattttccagccttgactaatttatttgccttcaatgatcctttgtttcctgctctgaatggccaatataacttcataatccacctattccctctggcattatgaagatcaagtgagataatatatttgaggctttcttgaactagaaagattatacaagcctgtaggcattattataattaccttctttttgttagtcatacttgatctcatcccaaggaatattatcggtttttccctgtttccttctatccattctcccatccccttagtgttagagacatgctgggaaagactcatttacacatcctaaagggcttgccttaatacgagacgctgtgctcatgaacatagtgagcgtccttcttaaaccagtggttaccttcagcatcaggggctgtatgaagcctaggtgagctctcagtcatcctgaagtattttctcagcaataaatggttctcaattcctagtacaagacacagtactagtaactgagaagaatgtaataagcaaaacaaatatagttgctgctcttgtgaaccttatatcctagaaaagtagatagatagtcatcaattgctcacaaatctatgagtataatgactatgatgagggagatgaaggagagcctgtgttcctgtgacaacatagataatagtgggagatggcttcatcagagaggtcttggaagacatccccaagggagtgatggctccatctgtaggataaggggagggcatttcaggtagttgtccctgtgatggaaagatgacagagctggaagaaggatagatacatagtactagcatagtacaagatttggatgggtaagaaggtatcagacctcttaggaccttgcagtccaggtgaagaattcagattctcatcctaaaaactattggttgtcattacttccaaaacataaagagggaaggtatctgcaatagatctttagctaacatgggaagaaaaggcagcaaggcccatgtttttcctttctccagataaaatttgtattagtcccttctcacactgttatgaagaaatacccaaaactgggtaatttataaaggaaagatatttaattgactcaaagttccacatggctgggaaggccccaggcaacttacaatcatggtggaaagggaagcaaacacatccttcttcacaagggaacaggagagagaagaatgagtgcccagcaaagggggaagtcccttataaaaccatcagatcttgtgagaactaactcaatatcatgagaacaggatgggggaaactgcctcccttaattcaattatctccacctggtccctcccacaacatgtggagatnatgggaactacaattcaagatgagatttgggtggggacacagttaaaccatatcattctgcctggacccctcccaaatctcatgtcttcacaattcaaaatacaatcataccctcccaacagtccccgaaagtcttaattcattccagcattaacttaaaagtccaagcccaaagtcttatctgagaanaggaaagtttctgctgcctatgagcctgtaaaatcaaaagcaagttagttacttcctagatacaatgggggtacaggcattgggtaaatatacccattccaaatgggagaaattggccaaaacaaaggggctataggcaccatgcaagtccaaaatccaatagggcagtcattaaacattaaagttccaaaataatctcctttgactctgtcttatatatatccagggcacactgatgcaagaggtgggctcccatggccttgggcagctccaccattgtggctttgcagggtacagaccccctcctggactggtattgagtgtctgtggcttttccaggcacacagtgcaagctgtcagtggatctaccattctggagtctggaggatggtggccctcttctcacagctccactaggcagtgctccagtggggactctgtgtgtggactcnnnnnnnnnnnnncaaccacacattttccttccgcactgccccagcagaggttctccatgagggctctacccccacagcaaacttctgcctggacatccagttgtttctatacatcctgtgaaatctaggcagaggttcccaaacctcagttcttgacttctgttcacccacaggcccaaaaccatgtgtaagccaccaagtcgtggggcttgcactctttgaagcatcagcctgagctgtatattggcaccttttagccatggctggagcagaagctgctggaaggcagggcaccatgtcccaaagctgcatagagcagagggagcctggggcccacccacaaaaccattgttcccttctaggcttccaggcctgtgatggaaggggctgctgtgaaggtctctggcatgcccttggggacattttccccattgtcctggtgattagcattcagtccctcgttacttatgtaaatttctgcaactggcttcaatttctccctagtaaataaggttttcttttctataaaaatcatcagactgcacattttccaaatatttatactctgcttcctcttgaacactgtgtcacttagaaatttattctaccagataccctaaatcatctctcttaaattcaaagttccacagatctctagggcaggggcaaaatgctgccagtctctttgctaaagcataacaagagtcacctttgccctagttcccaataagttcctcatctccacctgagaccacctcagcctggacttcactgtccatatcactatcagtattttggtcaaagctatccaacaagtaggaagttccaaactttcccatatcttcctgtcttcttctgagtcctccaaattgttccaacctctgcctgttacccagttccaaagtcgcttccacattttcaggtatccttatagtagccccccactctaagtacgaatttactgtgttagtcctttttcatgctgctatgaagaaatacccaaaactgggtaatttataaaggaacgaggtttaattgactcacagttctgcatggctgggaaggcctcagaaaacttatgatcatggtggaaggggaagcaaacacatctttctccacatggtggcaggaagaagtatgagtgaccagtgaaaggggaagccccttataaaactatcagacctggtgagacttaatcatcacaagaacaggatgagggaaaccaccccagtgattcaattatctccacctggtccctcccaggatacatggggattatgggaactacaatccaaaataaggtttgggtgggaacatagccaaaccatatcgaaatgcaacctaaagctactgagtccaagcaaaagtcaagtagactctctaatcccagataacctcagagaggaaatcttaaacccaaagaccaacttcccttttctgcctaattgcctttgttcttctccaagaacctctttggtcttggaaggatgctcattctccaccgtattttgcagtggactattgggaaggaaagttccccatccanggatgctatgtgcacacattcatgtgcttggacctcaccaaggatctcactggacccccttcgcggggcccatctgtgactgtcatcatttggctaggcaagcctccttgagagtaatcacactctggcatcggctgaatgtcaaggttgttattgcagttatagctggtgggatctttgtttctcaaatgaagctttcttattcataaagtgcagtcatctcttcctgctaatcacacccttgaaaaaagagctggatgtgttcctgagcagggaggacctctctgaaatcagcttagaggaatgagtgtatgtttgttcttcagtggtgtctagaatctgacttctggcaatcttttccaccatcagccctttctctttgccaggattcctttacctcctatgtcagttgaccttcctatgttgcactgttaccctgtgtttttcacaccctgtatgcaaaagagctggtcaccattttatgtaaaataaatttcaaaatacagaatttagctgagcaaaacagaacccgaagtttcatttgagactgaattttaaatctaatatttattcatgagtttttgcagtgaccttttcagcaccttttttagatcactttcttataataatttacacatattaagctaattattcagtatacttcacagaaggcttaaaaaatggaactgtaacaagatcttttgtgcaaattatgagaagctgtcttctgtttaaatttcagaatgcatatacttacatgatgggcgcttgtgtgattgtatttaggtttttctccatctgtggaaaacatgtaagtacattgttaagaatcttatcttaaaggtcatatatgtgttaatgaaagaatggtgattaattgattacagccttacctcggttatttagggatgtaatcacaaattactagacatcataagtcaagattaacaaatattaaacattgttaaatgaatcagtagcttagttatcattcgatgtgtttcctgccatttatgttgcttattctttatattcaataattaagtaaattatttaaatttattaatttaattaattaaagataaccagtcatgaaaacaaaaaccaatattctttcttaatgcatttttatttttattttcaataaagttaagtaacttcaaagaaaaataatctttaatgaatttttatngtttccatgattccagatnnnnttttttttttttttttttgagatggagtcttgctctgtcaccaggctggagtgcannngtggcaggatctcggctcactgttttctccacctcccaggttcaagtgattctcctgcctcagcctcccgagtagctgagattacaggcatgtgccaccacacccagctaatttttgtatttttagtagagacagggtttcaccacgttagccaggatgagattccagattttttaatattatttgttaatgtccatgggtatattccacatatatttttcattttttacaatgctgtttccttttttattacaaacgttatttcatatatacattccatatatacacataaccaatatacttaatatttgatagttatttagtattataccattgcttaggtttgtttttgtgaaagtctgtttgtggaagaaacctgagtttacttgctgtcagtcaaaaatgtgtagcccttatcatagagaattgaggagcagaagagaaaatggaaggtgaaaatgcttagtgttactcttattaatttgtgtgaagaggctgaaatgtttgaagtaaaaataaaattcgcccaacgcctggtgggttttgaagaatattttgatgtaataccttatagaaagcaactttcaaatgatacacctaagagtgattttgagccaatctgcttaaagattagttgctacccaggtcttccatagcttaatagattgaatattctctctctctctctctctctnngtctctgtctctctctctctctctctctgtctccccgcttcccacccctggtccccaggtaacgctaaagatgctcctcttgacagtggtcgtcagcatgtacaagagcttctttatcatagtaggcatgtttctcttgctgctgtgttacgcttttgctggagttgttttatttggtactgtgaaatatggggagaatattaacaggttggtatatacttatctttctattcatttatcaagcaaaccctttggtaataaaattttaaataaacaaatgttttttgtctattaaagtctaggagaggggaaagggaagtaatcagttggaaattaaaataaaatagccaagttttaaatgatatttttccagttgaaaaaggttattcaactgaaaaggtctactttcaaaaccttagaaaaattagaaataaaagaaaacaaaaagaatctataattccaccaacattcagtaatcatcactccnaattccagcactttttctatctacatattgccaaaaaatgatatattatttttagatggaacaattttaagataactctgggtttctttctcagcaaaatatgggaatttagagccccccaaatggttgtaatatatgaaaagcagttttcaacgatttgcctaagttttatagatgcatcattttttttcaagtcctgatacaacaaagtatataccgatatgtcaagttgtaggaattagatacatggctgccccaccttttatagcatcgtccaagtgaaagaaggaatcacttagttgccaaagcctgcctacttagtcattcaagatgaatggtaataaattagtggccatgctagagttgggtgttcagacggctatacttggaggcaagtataaatgacaaattaataaaaaggtataacctttggaaaaggtnacttagcaaccaaatttgctacaacctccacttgttttggtgttggcaaaccatgctatttcatgacaaacccttctataantcacattctgttaatttttttttcttaaattaggcatgcaaatttttcttcggctggaaaagctattaccgtactgttccgaattgtcacaggtgaagactggaacaagattatgcatgactgtatggtaaatatctctcatcatgaacaaggcttagacttagaatcagcatgaaactcttncaacacaatgttggcaggatcatccttgttagcgttaagactttatgtatgaaatataaatcctgacacttcagtccataaaattgcattattatacattccaatgtgtgaatatttagggcaaaatactcaattaacccaacttgagctattgtagttccattcaacaaantatgcattttgtgagtgttgttctttccctcccatctttaatattttggtaatgaaagtcaaacaggaagccctttaatttaatgtgttcagtggatagttcactgcccctcagctatatgctaggccctaggtcttcaacaactccaaggcacactcaaggagcttacctcaagggctctgagttactcttcctttatactataaagtatactttatacttccttcttactatatcctgtcttccccaagcatcatccttctggtctcagtttaaacaggtcttttgccttaagctaagacagccagctacatgttcttatgctgctctatagttctcctttgtaataaggagctgtagtgaattactggcttacattcatttattttatctctttctcaccgtaggacagaccaatgctgtgttaaaaaatgtctgcagtgatggaaatctgcactgtccaattcgggagccactagccatatgtggttatggagcatttaaaatgtggctaacacaactgaggaactgaattttagttttaattttatattattacattttaattaacataaatttaaatttacatngctgcatggagctagtggtcaccatattggactgcacagcactcaactgtcagtaggaagaagagatagggccgcctatgtctttcttaccagtctctccccaaacttggcacaaacctagcacacagtaggagtgctgggacttcatgcatgaattaacaaggagaatttgccaaatcaaatagaatacaaggcaaaatttaatgaatcttaaataaaaatacaagcagaatgctgggattatagaagagagagctagtttatctggctgagaaaaaccagaaagttctctcagaggtgtcggcnttttatagagcctgtaaacaatgagagagtttttgatgacttaagtcttgtgctattccatagcatcccataacagtgttccaagaatggggcttactcctcacacaatttgaatgtgaatttnctatgtgcaggcacatcttgttttactgtgcttcccttattgccttttgcagacattgcatttttacaagttgaaggtttatggcaatcttgtgtggagcaggtctatcagggccatttttccaatagcatgtttgctcactttctggttctgtatcacattttggtaattctcactatttcngactttttcattattatctgttttggtgatctgtgatcagtgatcttttgatgtttcttttgtagttttttttgaggggtgccatgaaccatgctcaataagatggagaatttaattgataaatgtcatatgtcttctgattgctgcaccgaacatctgtttctccaactcttctctcttttcttgggcctccctatgtcctgagacacaacaatattgacattaggccgattcacaaccctacaatggcctataagtgttcaagtgaatagaagattcacacatcagtcacttcaaatcaaaagctagaaatgattaagcttatggaggaaggcatgtcaaaagctgagttagcccaaaagctaggtcccttgcaccaaatagttactcaagttgtaaattcaaaggaaaagttcttgaagggaattaaaatgctactccagtgaacacatgactgataagaaatagccttatggctgatatggagaaaaaattttaccggtctcagtagaagatcagactagccacaacatgcccttaagctaaagccaaatgcagaagaaggcccttactctcttcaaatttatgaagggtgagagaggtgaggacgctgtagaaaaaaatgtttgacgctagcaaaggttggttcattatgtttaaggaaagaaagaagccatctccataatatgaaagtgtaagtgctgctgatataaaagctgtggcaagttatctagaagatctagctcaaataattaaggtggctacactaaacaatggattttcaatgtagacaaaacagccttctattggaagaagatgccctctagaactttcatagctagaaaggagaagtcaatgcctggattcaaagcttcaaataacagacttactctgttgttagggactaatgcagctggtgactttcagtggaagccaatgctcatttaccattccaaaaatcctaggaccctgcaatggtttgactgtgtccccacccaaatctcatcttgaattgtagttcccttaatctccatgtgtcgtgggagggaccaggcagagataattgaatcatgggggtggtttcttccatcctgttcttgtgatactgagtgagttctcactatagccgtcatgagatctgatggttttataagaggtttttccccccattttcttggcacttcttgctgccaccatgtgaagaagaacatatttgcttccccttccgccgtgattgtaaatctcctgaggcctcccaatccctgctgaactgtgagtcaattaaatctctttcctttataaattacncagtctcaggtatgtctttattagcagtgtgagaanggactaatacagtctcttaagaattatgctaaatctaccctgcctgtgctctataaatggaacaacaaagcctggatgacagaatagctgtttgcagcatggtttgctggatatttaagcccactcttgagacctactgctcagaaaaaaaagattgctttcaaagtagtactgctcattaacaatgcatctacctggtcacccaagagctgtatcaggagatgtataaggagattaacattgttttcatgcctgctaacacaacatccattctgtccatggatcaagaagtcatttcaactttcaagtcttattatttcagaaatagatttcataaggctaatagctgccatagatagtgattgctctgatggatctgggcaaagtatattgaaaaccttctggaaaggattcactattctacatgccattaagaacattcatggccaggcgcagtggctcacgcctgtaatcctagcactttgggaggtcaaggngggtggatcacgaggtcaggagatcgagaccatcctggctaacatggtgaaaccttgtctgtactaaagatacaaaaaattagccgggcatggtggtgggcgcctgtagactcagctactcgggaggctgaggcaggagaatggcgtaagcccaggaggcggagcttgcagtgagcggagattgcaccactgcactncagcctgggcgacagagtgagactccgtctcaaaaaaaaaaaaaaaaaaannnaacattcatgattcatggaaggaggtcaaaatatcatcaacattaacaggaatttggaagaagttgattccagccattatggatgactntgaggggttcaagacttcagtgaaggaggtcgctgcacatgtggttggtagaaatagcaagagaactagaattagaaatggaacctgaagacgcgactgaattgctgcagtctcctgatcaaacttgaaggaatgaagattgctccttatggatgatcagagtggtttcttgaaatggaatctactcttgcagaagatgctatgaacatagtcataatgacaacaaaggatttataatgttacataaaattactcgataaaccagcagcagggtttgagagggttgactccaaattcgaaagaagttctattgtgggtaaaatgctagcaaacagcatcacatgctgcagaaaaatctttcatgaaaggaagagtcaattgatatgataaacttccatgttgacttgttttaagaaatcgccacagccaccccaaccttcagcagccaccaccctgctcagtcagcagctgtcaacattgaggcaagaccctccaccaccagcagaagattacaactcactgaaggctcacatgattgttagcttttcttagcaataaaagtactttttattaaggtctgtacctttttggacataatgccattgtgcacttaataagccacagtatggtataaacatgagttgcatgtgcactgggaaaccaaaacatgcataggactttattgcaatattcactttatcacagcagtctggaactgaaactgcaatgtctccgaggtatacctgtagagtctatattcccaaatcttaacccattacttcagtcagagaaaaacttgcaaatgtctttttatttgattttctgcagtttgatcacagtggctacttatcacttggggctggtggtggtggcagaaagaatatctagtggagatcagttcattttcattgtgcttttaaaaatggcaataaccttgggattctacaagataacagcatagcataagtgaaatattcttctttatttctgctacagatgatgtacccaatgtatgtggcttttgaaagcttactttgaagttaattatatgctatatgtttaggaatctattcattaactatagtcagctaaaagaaatttgtattctctactcttatcctctccttcttccaccccaatgccccaaaggttcagcctccgttttgtactccagatgaatttacatactgggcaacagactgtggaaattatgctggggcacttatgtatttctgttcattttatgtcatcattgcctacatcatgctaaatctgcttgtaggtaagtgatgttagttgaatactttttactctttaaactcaggtacttagataattatgtattattttcaagcaattaccctggatttcttggggtgggtgtttcatggcattaattataaattgttgtctctcaatccagaagctgaaaagacctttgaaaggcacgtaaatgacagttgactgcaattttaagagttgtagcatttattatgatcattaatttcaaaagggctcttctcatggattgagtngaacaaatatcagagaggactatctttctagccttgaaatatcacatttatgtttttgaaaggtacaccttatttacccagatgtgggcccacccagaactcaatttgacctttaaaaatggccctggaactctcaactaagagttaaagccagctggcatgactatctctatacacagaatgtgtgtttgtgcttacgaccctcatgttttggaaaggcaggagctggagaaatagcttagtccaagtgtaaacatgccaatttggaaaccatactaaaagcactttaaaaatttggcttatgatataggtgatagaatccattattaataatagatgttttactctttctaaaatatgacagattagcataatggtctaacttctgaatttttctttttttaaaagccataattgtggagaatttctccttgttttattccactgaggaggaccagcttttaagttacaatgatcttcgccactttcaaatmatatggaacatggtggatgataaaagagaggtatgggagtgggttgttgtttcatgcctcacctcaaagagacttatcaacatgtctcaaacacttaaatggttttcaggttggcaaatgacacagattgaatttttaaatggnntgaanttgcagaaagccatcatttacagttcagcagaatcctctaggaagggagacagcatcgttcctccttacatgatcttcaaccatgaacatcctcttcttcagactttccgaaggccagcagggaaaggaagccaaagggactctgtaggaatcagccttgggctcaggccagaaaaagaggtgacactgaaactaccaaagggacagtatattancaaattcatgatcacctcaaactcaagtagcaatcccaattaaaagtacaaaggagttatttaagtggcatctgaaggtgtgtattggattatttttgagttactacttgaatgttttaaccataaagtttacccaacgtttcttaaaattagccaaagtagctgcagtctgctcttcctcttcttgtcattgtcgtcatcaacctccacctccttcccttcctcctactcctcctcctctaccccctctnnnnnnnnnnnntcttccccctcctcttccccctnctcttccccctcctcttccccctcctcctcgtccacctcctcttccacctcatcttcccccctacctcctcctcctctaccccttcctctaccttctcctcccccacctcctccttctncacttcctcctgttcttcctccctctcctcttctctcttcctcatcctagcaaacactcaggcacagttctaggcactgaggatatagcagtgaacaagatggaacagctcctgtactcacagagcttacagtctaataggtggagacaaataataaaaagtgaaataataataaatagtgcagtatttccagaagttaaaactatagacagtagttggggttggggctgcgccggcaggaatgaccatgaaggaggcctgaggggtgctgtgggacctcatgtctgaacaacaaggaaagttctgccagggtatttggggcgagatctggaaatagttcaagaagagttgatgaagggcacaaagaccaagaagccagcctgggcatcataaatgaggggctccggcagtgcactcaccccaatttgctccctcttcttggtacccctgcccgcccaggcttcagtcttgagggctttcctttcaaggtagtgagagccctgcgtgcagggcatctgagctcactctcagctcctcaggatccacctgtctctctgtaactgtcacccacgggaacctgctttcatgcaatacacacatgaagacccactgccctgcacaccgagatgtcagtaactggcaggcagtcatggtcgccagtgttccgcccagcactttcattcaaagaaagatacagccagggggcatggtggtttgtgttgcatggaaggtagcaacacatcttagattctcagtgtctggatcagttctgaccccagaagagggcagagctgaaggcctaattctcttgtatatagtttttctaatcctgaagaagacatgaaaaggagttagttagctctttttttttttttttttttgagatggagtcccactctgtccccaggctggaatgcagtggtgcaacctctgcttcctgggttcaagtgattccccagcctcagcctcccaaatagctgggactacaggcatgtaccactatgtccgggtaatttttttatttttagtagagactgggcttcatcatgctggccaggctggtctcgaactcctgacctcaggtgatccacccgcctcggcctcccaaagtgctgagattacaggcatgatccaacacgagttatcttttaatggtagaaaaacatgtaataaacattgagacgttcctagcatcccgattgagtgaagaggaggattacacgtgtgtgtcttgtccattnccttgtgtgagtacattatggcccactcagtgcatgtgttggaggtggcgtctgtgggacagnctatgcaagcatcagcctggtgagagcaagagtagggtcctggggggctgaaggggagcttcccatgcctcactccaaatgctgcaatgtggtgacatcccagaaagagaagcaccagcagaaatttatcactcagttttctatcctctgctcttcttagtgcacttaattctaccaggctccaaggaggtgaccagctttcttcacatctggtgctaattatgctagcttttgagaaatagcacctttttttttttcttttgagaaacccaagtgaatcatcgaggcctgatgatttgctacttcttgtgctctgccccgcagggggtgatccccacgttccgcgtcaagttcctgctgcggctactgcgtgggaggctggaggtggacctggacaaggacaagctcctgtttaagcacatgtgctacgaaatggagaggctccacaatggcggcgacgtcaccttccatgatgtcctgaggtacaaggcaggctttcgcagggctgaatgggggccagaaaccacagctgtcagccttcaaggcctctgcagtacaattaacaagtgtgtttccaacccaaaatgccacatctgctgtggtcgtgaagtggggtcacatgtcccaggcgctaagtgccctttcctgccgtcctccagtgaacatttccaagtaggtgcctcgcaggggagggctgggcggctgtgccctgatgtcccgtagggacccctgcccactgaggtgcaccccgaggggagaggtggaagggatctgggagagcacagtcccaccccttccctgtgatgcgggaactgcccagaagagctgagtgacttgcttgcggttacagggctctggcaaagctgggactaacatgcagagcacctcacttgcatgggtctgatgagaatttgcctggcctgctctgtgatggttgccccgttggctaaaacaccagattttccagacatttcatgagcgcccagacaggacagagggtcaaatggagtctctgccttttcttcttcctgggagacttctgtatttgtttccttgggctactgtaacttagcaccttaagctgagggatttaaacaacagaaaggtatggtctcgaagtcctggatgctggaagcccgagatggaggtattgcggggctgattccttctgaggcctgtgagggagaatctagcctagcctctcccctggctgctggaggtgcgctgccaatgttggtcattccttggcttgtagaagcatcagcttggtctcccttcatcttcacatggcattctgtttgtgtgtgtgtgtgtgtgtgtgtgtgtgtgtgtatctatgttcaaattcctccttttataagaacatctgtcgtattacattaggagtccattctactccactgtgatctcattttaacttaattactaattacatctgcaatgttctatttccaaataaccttacgttctgaggtactagaggttaggactccaacatatctttttttgggaggaaacatttaacccataacagcttcttaagcaggagtccatggataaaacagaggcccatggacttgcaagggggaaacaattacatctttattttcactaatcactctttgaaatgtgtcattctcttccatgatgaacgtaggcaacaaaccacagtcgcattagcaatatccatgtctttgttaccaggagaaatcatagatattttcagatgacataactgactgccttgactgacatcttaaaatatcatttatgctcaccagtgcttctgagttatggtagtcatttgagctctgctagatggtattatttaatggaataataaaggaagcacttacttatgttactttatcactgccgttttaaagtaaatttgagaattgtattttaatataattggtttcctttgtaatacggtgtatttcattccgtgcatttaaagcattatttcaagaaatgtcccataagctccactgccacaggggtccatgccacagaaaaaaataagagcgactgactagcagtagctattttaatcacatagcagtacctacaaggagtgtgtgggatattgtgttgtgtctcaaatttcaggagaactcaggtgtacagaaatatgcttgtggccagaagctgaaaaaaaaaaaggatcagatcagcacaggaagaatctgggaaaagttctctcaagttatcaatttcacctcccactatccagttcttccaccaacaagctaaaaagcagaaaaacatgggacggagaaatgcatgtgaatcacaaaggaggtcatctctgtcagaatgtttctgagtctttcctcctagtggcaaatttttttggttgacatgaggcagataggcatactgttgtcattaataattaatattcagcctctttggaacacaccgatgtatctaataaaaatagacccaggcaaaatctgtgatggggctgtctctccccttcctatttattcccctagcccagtatatgataaaaacaaacagtgaagataaaagcaatgaagctttgatgggattcttgatgagtcagggaagagaggaagagaaaattcattttttttccctctgtggtgggaagagatttagcaaagagaagcccaatgtaccagtgctctcaagagcctccaggctgtgatttgggctctaggatggaactgagctcacagaagtgggtagctcacaggtgcaccagggtgcatgggtctgagccataacctggaatcctgtggctacacctgggtgcatcacactgccaagaggggctgtcttctcctctggtggtggactgctcagtccaggcatcctgggagctgccttattgccccatgaactgtaatgctgtgttggctttacctacaactgcatgggcctgctgtgtgtcaagggttaaatgggagtggatattcaaccctgggtttgtttggtagcctctgaaattaataaggaagaccaggcatggtggctcacacctgtaatcccagctctttgggaggctgaggcaggtggatcatttgaggttaggagtttaagaccagtttgaccaacatggtgaaaactcgtctctactaaaaatacaaaactagctgggcatggtggcacacacctgtaatcccagctacttgggaggctgaggcagaagaaatgcttgaacccaggaggcggaggttgcagtgagctgacagcgcgccattacactccagcctggtcaacaagagtgaagctccatctcaaaaaaagaaaaaattaattaataaggaagatacagaaggatgaatgagagaaacagagagggaagtgggatgcagggagtagggatgtgtgacgcacagccgagcatattaaccatcacctcggggggctaagtgtgaagaaagtatgggaggtgcggtcttccttctgcagagaaagtcgcgagccttttcctagcctggatcttttgctttacaggcacttagatggcacttactactataagagcctcacagatattaactcacttagtcattaaataaaggagttaccaggactatctatcatctttgttttacagacgagggaagggagacacagagaggtgacaggaccagcccaagacatgcgggtggcgaggggtggcgtcaaaacactagacttattagactgagtgatgtcctctctggattttgacgtatctaagggtgtaaatctcaggtgaatcagagctgcgattgcaggtgtgtttctcccaccagcatgctttcataccggtccgtggacatccggaagagcttgcagctggaggaactcctggcgagggagcagctggagtacaccatagaggaggaggtggccaagcagaccatccgcatgtggctcaagaagtgcctgaagcgcatcagagctgtgagtgaactgcacacatgcaggttgggtccccgccccgccccgccctgccccgcaggaggatgaaagaaaagagcttcgtgaacatgggattagctccttctgtgatcagccttctatccctttaaactatgtccactacctccacctacagtggtcagatcatgatgcagactttcactctagtccacagtcagccctgccctctttagcagagtccattaggagtttccatggaatcccatttttataatatgacatgaagacttaaaaagcagtttattggccaggtgtggtggctcatgcctgtaatcccagcactatgagaggtcaaggggggagctcaactcacttgaggccaggagttggagaccagcctgccaacatggcaaaaccccatctctactaaaaatacaaaaattaaccaggcgtggcacacatgcctgtagtcccagctactcaggaggctgaggcatgagaatcacttgaacctgggaggtagaagttgcagtgagctgagatcatgccactgcactccagcctgggtgacacagtgagaatctgtctcaaaaaaaaaaaaaaagtttattgatattaaaatatgatgtccatttattaaactcaactgttcaaaattctagaattttccacatattgaatttgcaaaactactttaaattgctgctaaaaatttcaacaaactgtgataatgtcatttattctgaaaagaccacctctcaagattaatgatgtttcctgcttcctgacttataagcctataagattctctaactcataaattaatgaatgtatactgcattgacatatactgtatttacagtgaatgtttctatgagataattacttttatgaatgcttttggcatgtttcacagacagggaaaaaacccaaagcaatcactaaacatttatatataacagagattgtttcaatcctcttcctcagagcaacaacgtcaattgcagaaacatgtatttctattggaggagaaaaggttcccagagaaaagatacaactcaacctcctatagcaaggccagctgcccagtgatgtcccatcactgctggccactgtagctatgttagctccagggatcaatctgtcctcctctggctctgagagggcacaaggagtcctggctttagtatatgccgagacagactggtctcctggattgacctgaaaacttcagaaccaagacaagcacaaaggttttctcatccacccaaggctctttgcctcttacaacttgcaggaaagtgggaaaggggttgggattgtattggcttaatgagcctagaataaaataagaggagaaaaaatagaatagccacattagtaattttaaataagacataagctgggcacagtgtctcatgcctgtaatcccagcacattgggaaactgaggcgggaggatcgcttaagaccagaagtttgaaactagcttgggcaacagagtgagacctcctctctngnaaaaaaaaaaaaaaaaaaaaaattaattagccagccacagtcatgcacacttttagtctcagcttctcaggaggctgaggcaggagcatagcttgagcctaggagtttaaggttacagtgagctataataatcgcaccactgaactctagtctacgggacatagtgagatcctatctaaaaaaaaaaaaaaaaaaaaaaancagaaaacaccaacaacaaaaagaaaaacaaataaataaaatacaagcatcaatgaccgtcactattctgattgttacacaaatgaaaatggactgggaagtcaatgcatacctctctaagcccacctttgcatcagcctaccttaattgaacattgaaattagttcaggatccttacttggaaattcctaagaggtcacccgtcatgcaggtcttaggagaggggagaagaaaataagatttgtgtggcggtggggcaggcaggatgctgggcttattttactgaacaaacaatcgcatttccatagaaacagcagcagtcgtgcagtatcatccacagcctgagagagagtcagcagcaagagctgagccggtttctgaacccgcccagcatcgagaccacccagcccagtgaggacacgaatgccaacagtcaggacaacagcatgcaacctgaggtatgggcacgaggcgtcctctgtcccaggacactctgtgggcttctttcttttaataaaaataaataaaatgggtgctggtcagatcattcaaacaaggtgacaatatttctaataggaatattttcattaaaaaaaaaaanggcaacggaaatccattgatccagattatgtctatgtctttgccatcttggatgcagttgaggttatttaaaaagagtctgactcatatgaatgccaaaaaaagtgaatgccctgaacttcataattctgtagttttatttttgtctaaagagttaacatcagctctaagggctgcatttgaatctgggtatattacatggagtatggtttacaggaagctctgccctaaatattttcctcccttctctaaagtcatagaaatttgcagtaaaatcagtgaaaatgtttgtaattagctttcaacctccttaaataatcagactcattgaagatatatcctttcctggttgggtatcagaagaacatatggaattcaggacaattggcttgaggagtttctatttattaagtcacagcatattttcattttcaaactctatgtatgtgtctatataaaaaagtacaaggagttatcagcgtcctggagagtcctgtgctgtgagaagcagctggctgggaggacctgcccctttatgaggcagcaattacgatttgaggttaacaacgtggagctgatcccgtgctctgtgcagagcccccagcagcaccaggctccatggtgtgcagcaacgcagccaggggccagagtacctttggaagcctaaggctccacggcccagcagggtgctccaggaggcccactggtgccatagctcagttttcagtagatctcatgttttgggctccccagcctttagaaatcatctgccaccgactgcagggacttctgcggagttagtcaaccaaggggcaaggggaggcatctgcagtggccttgccactggtcaccctgaagatggttggcaatgtctcgcatgggagagggaggcaggctgaagtcaggacagacagtgagacagggctaatgaggttggagaaagtgatgatttcagagccctctgtttttagatgaaaaagcctaagaaagaaagaggtgacnnnaaaaaaaaaaaaaggatactaaagagatgtgaagaccaaggcttttgttaggaaagggagaggtgacaggtgaacctgagaacaggtgaaatgtgtgggaagtcttggcatctctctttctttcactgtagcttagattgttatcagctccatcctccaccagggcccatcatacagctttgcctgcagagaactctttccctgaagctctaagtgtggatctccaggctggtgacagtagactgcccccgcccagccggtccccactgccccaccttccttcctgcagttccccttccttaccccctggtttgccagcgaagatatgaagggaaacaatgacattctcaggcatggaggggaacgaagcagtgggggttacccaggacaagatgtcccaggaagagtggtgtctccttcagcagggcgtgcagtggtggctctttttcttctgtgaccctttatacccattttcacttttccccattgtggacactctgagtccatgttgttcttcccacttgtcctcctgccatgtgttttcctttcttatagtaaaaggaggagagcgcagggttaaaaagacggtaactgtgtgcttctcctcccatgcagacaagcagccagcagcagctcctgagccccacgctgtcggatcgaggaggaagtcggcaagatgcagccgacgcagggaaaccccagaggaaatttgggcagtggcgtctgccctcaggtaggtccatccaggcatttctccagcgatcgaggcattttaaaggtatttactctgtttgtgtgttttgggtcttgcctttcaaaatgcaagtctgcatctacagttgtttacagacagcaacataatgaaaaatgtagtcttgtcaaaaacattgtcccccaaaataacttctctaaatatgacttacattagcccccattttccgggtacatttcaggctatcatggttgagaagccagcacctatgaaaaagacaagattcagnaagagggagaaatttccaagggctttctgtgtgcctcaaggcatgctcaaacatgggcaaaagtattcaactgaagagggagtgggcagatgcaatcattcagaaaatgccacgaagttctcaaaaaagggacaggcccccgatttctctcagcacactttgcagctgagccggtcactggctttaggcaagctgctttacctccatacaataacaannnncaacaacaaacacttacgtagtactcactgtatgccagtcactcttctaagtgcattacttgtgttctctcattcaatcctttaataaacagtcccattattttccatattttactgattcagtaagagaattttcttctatgagcttcttctctatagggttttggaaagccaggataatcccattaagagcactcaggaatsgagggagagcaatgctccccacagcccagtgtccttgtacatgtttttttctatagataactaagttcatgctaaagcaggagctttataatgctctttaactgtgccccaacttcagcccaattgaaaggagaagatgtgtagcatatgtgttccacaaagcagatgacagcacagcttacattttgaggctgacgatgttcagtgggtcttcactgggactaccaccaaggaaagtatcccctttcatatccaggaacttatttttcagagatcagagaagatctaggttcctcctgattcaaacacagcagagaatgacagcatcaagacaacgtagatggtggtgccaggtcataaattacaagctgagtcggttcaattttatcctgtaggcaattaggagctagcaaagatttctgagcagtatgtgacttttggaatctgtgctttaggaagttgacttggcagcaaaggagggaattgtctatgacagagcctggaggcaggttacaagctggaggaagttaccgtggtgtgagcacaaggcaacaaaggcatggacagagctgggggccagatactgcagggacagataggagaggtgacctggggagaatgcacagtccttggtattcatttaaatggacaaaagaaatgatgctgtcaaaagtacttccattgggccaggcgcagtggctcacccctgtaatcccagcactttgggaggccgaggcaggtggatcatttgaggcctggagttcaagaccagcctggtcaacatggtgaaaccctgtctctactaaaaatacaaaaattagctgaccggtagtggtgtgcacctgtaatcccagctgcttgggaggctgaggcaggagaatcacttgagcctgggaagcagaggttgtggtgaaccaagattgtgccgctgcactcctgtctgggtgagagagtgagaacctgtctcccaaaaaaaaaaaaaaaaaaaaaaaaaaaanngtacttccactggtccatgcctaggtggtccccaccaaaaacggtgatcgtgaaatagacatgctcttggggtcaggctgagtttgaggaggcagaggagaagaaaaatagtattttgagggggcagcaacaatgtcacaggtgtcatgctatgcactttcctaagtcatctcatgtaacctgttttaatcctgacaatagtctaattaggacattttcattgtcttcatcttacaatggaggaaacttgggttcagagacgtgaagttgcccatagctacatatccacaggttgcagaggtgggtcccaacaccatgactgtgaggccccagaaccacacctttcctcccataccctcactgcatcccccctcgcctttactgtgggtcacactctccttcctttttgttttttttattgcatatgctaatgaatgcacaggtttctgtgacacttaatagttcccgaattggaatttttttagtgattttctagtttttttaattatttcaaaatgaaaggggtgagcagcacgtttttttcccaaatgacatcattagttgtccatgagcacaattaaaaaaaaaatctatcgtgaacataaagaatcaagtcctgaaaaggatgtttacttagcatctgtggctaggacacgttagccaaaagtatcactgatgggaatatcatgggtttttaggagtgattacaatagggtgaagcaataccaagtggctcataggactcattaattccacagccccaaaaccaataagccattcagtgtcctcagtcaacttacggtttggaggaaggacaaccatgaaatctgtcgtgtgcaaaatgaaccccatgactgacgcggcttcctgcggttctgaagttaagaagtggtggacccggcagctgactgtggagagcgacgaaagtggggatgaccttctggatatttaggtggatgtcaatgtagatgaatttctagtggtggaaaccgttttctaataatgtccttgattgtccagtgagcaatctgtaattgatctataactgaattccagcttgtcacaagatgtttataaattgattttcatcctgccacagaaaggcataagctgcatgtatgatgggttactatcaatcattgctcaaaaaaatttttgtataatgacagtactgataatattagaaatgataccgcaagcaaatgtatatcacttaaaaatgtcatatattctgtctgcgtaaactaaggtatatattcatatgtgctctaatgcagtattatcaccgccccgcaaaagagtgctaagcccaaagtggctgatatttagggtacaggggttatagctttagttcacatctttcccatttccactagaaatatttctcttgagagaatttattatttatgattgatctgaaaaggtcagcactgaacttatgctaaaatgatagtagttttacaaactacagattctgaattttaaaaagtatcttctttttctcgtgttatatttttaaatatacacaagacatttggtgaccagaacaagttgatttctgtcctcagttatgttaatgaaactgttgcctccttctaagaaaattgtgtgtgcaagcaccaggcaaagaaatggactcaggatgcttagcggtttaaaacaaacctgtagataaatcacttgagtgacatagttgcgcaaagatgttaagtttcttaagaaaccttttaataactgagtttagcaaaaagaataaaactatatagctcaatttatttaaaaaaatctttgcatgtgtgatgttatcattggcttcatttcttacccaaggtatgtctgttttgccataaatcagcagagtcatttcattctgggtgatcctaacacaccattgctatgttagatttgaaatgacatctctgttaaaagaatcttctatggaaataatggtgccctgcaaaatcttcctctgaactcacaggttagggatcacacaacttacttaatcgttttttgtttttgttttttttccttatatgtcaatggcccatgtcctccgggaaaattagaaaagcaaaatgattacaaagtgctgttagatttcttgtgctgggccagccaagtagaagtggacttgacttggacctttaactattttattacagattggacatttgctgttcagatgttttttaacagagggattatctcagaatcctgtgacctccaggttgttttataatctatttttctctatttaacattcctcagatagataggcaaataggacattccttctgtgtcacagaagtatcgtggtagtggcagtctacagtttatatgattcattgtaactatgagataaagaacaaccagtcatgtggccaaaaggattagatttgatttgatgttcacttggagtttactttttgtacatacaagataaaataaatattggatttgtaaaataatttactctgagttgaccatttttaaacccttttcttggtggggcaggcactcggtagcagcacattgcaaacctggattgaatattcagtccataattagttggcaaaagctgtgtccctgatactcatttcaactcaattctctgagccagaatgttcctttcacacttgtgctgataaggctgagtgaataccctgtgccctctaagcctacaatgaacagaaaaggcacttacaggtccagggcatgggtctgtactagtgctgcataaaaacaagatgtttttggctcattacacttgagtaatacagcttcattctcacgaaactgctgagccctttctaaaacgtctggaattgatgttcagcagaacatcattgttgttcattctgcagtgaagccaggggcagaataccaatgtatttaaagccatagcaccctatattttcaatgaagcgaaacagtcacacacttcctgtaggactcctctctacgattttcctgaaacaggtagagcatatgcaagccagagaaattaagcgccacccccatttagattttacccctagatatgtaaacatatgaaaaatcccattacacagatgggaaaacttgggtcacagtgttccctggtccgtagggaagccagacactgatactaatcctgtcatcacgccacccactgcctttaattagagggtgctgtcagttccaaaggcaccagaaagcatatggggtggaatacttacttgactcagagcccctgagagagcaggccacacatgtgtgacctgtatggcacttcaaggagactcaaagggaacaaggatggacagaatggaagggccaatgctattcactctannnnnnnnnnnnttttttttttttttttgagacggagtcttgctctgtcacccaggctggagtgcagtggcacgatctcggctcactgcaagctccacctcccgggttcacaccattctcctgcctcagcctcccaattagctgggactacaggtgcccgccaccatgcccagctaatttttttgtatttttagtagagacggggtttcaccatgttagccaggatggtctcgatctcctgacctcgtgatccacccgcctcggcctcccaaagtgctgggattacaggcgtgagccaccgtgcccggcccactctattcttaatggtacttccagaccaacatacatcagcagtgtctggctggctgtgccttggccaagctgaatgagataagggtacatagaagaaaaaccagtgttataaagccttacaggaaagcagctttaagccagtggaatgatgtgtttgcctgtttccttgtaggaagtggcattctatacatgggtggttttcagtatatcagtaagcagtggaactgtttctaaggccttcaacagatcccaaaattttaaccagccaaaagccaggcagccctcgttgaagttgaggtgaagatgaaaacggcactcatgtggtctcttctcacccactattatgggttgaagtgtgacccccagaaggataggttgaagtcctaacccttagtacctgtgaatatgaccttattaggaaatagggtcttcgcagatataattacttatgaggaggtcatactagaggagggtaatcccttactccaacatacctggtatcctctgaagacacaggcatgtggggaggatggccatgagacggtgaaggcagaggcatcggagctatgctgcccagagccaagggacacctgggctaccagaagctgcaagaggcaaggaaggcgcctcccagaggcttctaagggagaatggccccctgtcaacaccttgattctggacttctagccagaagaactgtgagaacatacatttctgttgttttaagctgcccagtttgtctgcttttttatcattttatcattttagttttttagtttagtctctgtcttctactaactccctttccaattctactttag

SEQ ID NO: 4 - DNA, 6799 bp, Homo sapiens
  5′UTR 1..65
  CDS 66..5282
  3′UTR 5283..6799
  polyA_signal 6081..6086
  polyA_signal 6777..6782
  allele 1658: 99-79316-158   polymorphic base C or T
gacagctctgagcgctggggttacagactgtggttttgtgcttgctcaccaaagctaacctcagcatgctcaaaaggaagcagagttccagggtggaagcccagccagtcactgactttggtcctgatgagtctctgtcggataatgctgacatcctctggattaacaaaccatgggttcactctttgctgcgcatctgtgccatcatcagcgtcatttctgtttgtatgaatacgccaatgaccttcgagcactatcctccacttcagtatgtgaccttcactttggatacattattgatgtttctctacacggcagagatgatagcaaaaatgcacatccggggcattgtcaagggggatagttcctatgtgaaagatcgctggtgtgtttttgatggatttatggtcttttgcctttgggtttctttggtgctacaggtgtttgaaattgctgatatagttgatcagatgtcaccttggggcatgttgcggattccacggccactgattatgatccgagcattccggatttatttccgatttgaactgccaaggaccagaattacaaatattttaaagcgatcgggagaacaaatatggagtgtttccatttttctacttttctttctacttctttatggaattttaggagttcagatgtttggaacatttacttatcactgtgttgtaaatgacacaaagccagggaatgtaacctggaatagtttagctattccagacacacactgctcaccagagctagaagaaggctaccagtgcccacctggatttaaatgcatggaccttgaagatctgggacttagcaggcaagagctgggctacagtggctttaatgagataggaactagtatattcaccgtctatgaggccgcctcacaggaaggctgggtgttcctcatgtacagagcaattgacagctttccccgttggcgttcctacttctatttcatcactctcattttcttcctcgcctggcttgtgaagaacgtgtttattgctgttatcattgaaacatttgcagaaatcagagtacagtttcaacaaatgtggggatcgagaagcagcactacctcaacagccaccacccagatgtttcatgaagatgctgctggaggttggcagctggtagctgtggatgtcaacaagccccagggacgcgccccagcctgcctccagaaaatgatgcggtcatccgttttccacatgttcatcctgagcatggtgaccgtggacgtgatcgtggcggctagcaactactacaaaggagaaaacttcaggaggcagtacgacgagttctacctggcggaggtggcttttacagtactttttgatttggaagcacttctgaagatatggtgtttgggatttactggatatattagctcatctctccacaaattcgaactactactcgtaattggaactactcttcatgtatacccagatctttatcattcacaattcacgtactttcaggttctccgagtagttcggctgattaagatttcacctgcattagaagactttgtgtacaagatatttggtcctggaaaaaagcttgggagtttggttgtatttactgccagcctcttgattgttatgtcagcaattagtttgcagatgttctgctttgtygaagaactggacagatttactacgtttccgagggcatttatgtccatgttccagatcctcacccaggaaggatgggtggacgtaatggaccaaactctaaatgctgtgggacatatgtgggcacccgtggttgccatctatttcattctctatcatctttttgccactctgatcctcctgagtttgtttgttgctgttattttggacaacttagaacttgatgaagacctaaagaagcttaaacaattaaagcaaagtgaagcaaatgcggacaccaaagaaaagctccctttacgcctgcgaatctttgaaaaatttccaaacagacctcaaatggtgaaaatctcaaagcttccttcagattttacagttcctaaaatcagggagagttttatgaagcagtttattgaccgccagcaacaggacacatgttgcctcctgagaagcctcccgaccacctcttcctcctcctgcgaccactccaaacgctcagcaattgaggacaacaaatacatcgaccaaaaacttcgcaagtctgttttcagcatcagggcaaggaaccttctggaaaaggagaccgcagtcactaaaatcttaagagcttgcacccgacagcgcatgctgagcggatcatttgaggggcagcccgcaaaggagaggtcaatcctcagcgtgcagcatcatatccgccaagagcgcaggtcactaagacatggatcaaacagccagaggatcagcaggggaaaatctcttgaaactttgactcaagatcattccaatacagtgagatatagaaatgcacaaagagaagacagtgaaataaagatgattcaggaaaaaaaggagcaagcagagatgaaaaggaaagtgcaagaagaggaactcagagagaaccacccatacttcgataagccactgttcattgtcgggcgagaacacaggttcagaaacttttgccgggtggtggtccgagcacgcttcaacgcatctaaaacagaccctgtcacaggagctgtgaaaaatacaaagtaccatcaactttatgatttgctgggattggtcacttacctggactgggtcatgatcatcgtaaccatctgctcttgcatttccatgatgtttgagtccccgtttcgaagagtcatgcatgcacctactttgcagattgctgagtatgtgtttgtgatattcatgagcattgagcttaatctgaagattatggcagatggcttatttttcactccaactgctgtcatcagggacttcggtggagtaatggacatatttatatatcttgtgagcttgatatttctttgttggatgcctcaaaatgtacctgctgaatcgggagctcagcttctaatggtccttcggtgcctgagacctctgcgcatattcaaactggtgccccagatgaggaaagttgttcgagaacttttcagcggcttcaaggaaatttttttggtctccattcttttgctgacattaatgctcgtttttgcaagctttggagttcagctttttgctggaaaactggccaagtgcaatgatcccaacattattagaagggaagattgcaatggcatattcagaattaatgtcagtgtgtcaaagaacttaaatttaaaattgaggcctggagagaaaaaacctggattttgggtgccccgtgtttgggcgaatcctcggaactttaatttcgacaatgtgggaaacgctatgctggcgttgtttgaagttctctccttgaaaggctgggtggaagtgagagatgttattattcatcgtgtggggccgatccatggaatctatattcatgtttttgtattcctgggttgcatgattggactgaccctttttgttggagtagttattgctaatttcaatgaaaacaaggggacggctttgctgaccgtcgatcagagaagatgggaagacctgaagagccgactgaagatcgcacagcctcttcatctcccgcctcgcccggataatgatggttttagagctaaaatgtatgacataacccagcatccattttttaagaggacaatcgcattactcgtcctggcccagtcggtgttgctctctgtcaagtgggacgtcgaggacccggtgaccgtacctttggcaacaatgtcagttgttttcaccttcatctttgttctggaggttaccatgaagatcatagcaatgtcgcctgctggcttctggcaaagcagaagaaaccgatacgatctcctggtgacgtcgcttggcgttgtatgggtggtgcttcactttgccctcctgaatgcatatacttacatgatgggcgcttgtgtgattgtatttaggtttttctccatctgtggaaaacatgtaacgctaaagatgctcctcttgacagtggtcgtcagcatgtacaagagcttctttatcatagtaggcatgtttctcttgctgctgtgttacgcttttgctggagttgttttatttggtactgtgaaatatggggagaatattaacaggcatgcaaatttttcttcggctggaaaagctattaccgtactgttccgaattgtcacaggtgaagactggaacaagattatgcatgactgtatggttcagcctccgttttgtactccagatgaatttacatactgggcaacagactgtggaaattatgctggggcacttatgtatttctgttcattttatgtcatcattgcctacatcatgctaaatctgcttgtagccataattgtggagaatttctccttgttttattccactgaggaggaccagcttttaagttacaatgatcttcgccactttcaaatmatatggaacatggtggatgataaaagagagggggtgatccccacgttccgcgtcaagttcctgctgcggctactgcgtgggaggctggaggtggacctggacaaggacaagctcctgtttaagcacatgtgctacgaaatggagaggctccacaatggcggcgacgtcaccttccatgatgtcctgagcatgctttcataccggtccgtggacatccggaagagcttgcagctggaggaactcctggcgagggagcagctggagtacaccatagaggaggaggtggccaagcagaccatccgcatgtggctcaagaagtgcctgaagcgcatcagagctaaacagcagcagtcgtgcagtatcatccacagcctgagagagagtcagcagcaagagctgagccggtttctgaacccgcccagcatcgagaccacccagcccagtgaggacacgaatgccaacagtcaggacaacagcatgcaacctgagacaagcagccagcagcagctcctgagccccacgctgtcggatcgaggaggaagtcggcaagatgcagccgacgcagggaaaccccagaggaaatttgggcagtggcgtctgccctcagccccaaaaccaataagccattcagtgtcctcagtcaacttacggtttggaggaaggacaaccatgaaatctgtcgtgtgcaaaatgaaccccatgactgacgcggcttcctgcggttctgaagttaagaagtggtggacccggcagctgactgtggagagcgacgaaagtggggatgaccttctggatatttaggtggatgtcaatgtagatgaatttctagtggtggaaaccgttttctaataatgtccttgattgtccagtgagcaatctgtaattgatctataactgaattccagcttgtcacaagatgtttataaattgattttcatcctgccacagaaaggcataagctgcatgtatgatgggttactatcaatcattgctcaaaaaaatttttgtataatgacagtactgataatattagaaatgataccgcaagcaaatgtatatcacttaaaaatgtcatatattctgtctgcgtaaactaaggtatatattcatatgtgctctaatgcagtattatcaccgccccgcaaaagagtgctaagcccaaagtggctgatatttagggtacaggggttatagctttagttcacatctttcccatttccactagaaatatttctcttgagagaatttattatttatgattgatctgaaaaggtcagcactgaacttatgctaaaatgatagtagttttacaaactacagattctgaattttaaaaagtatcttctttttctcgtgttatatttttaaatatacacaagacatttggtgaccagaacaagttgatttctgtcctcagttatgttaatgaaactgttgcctccttctaagaaaattgtgtgtgcaagcaccaggcaaagaaatggactcaggatgcttagcggtttaaaacaaacctgtagataaatcacttgagtgacatagttgcgcaaagatgttaagtttcttaagaaaccttttaataactgagtttagcaaaaagaataaaactatatagctcaatttatttaaaaaaatctttgcatgtgtgatgttatcattggcttcatttcttacccaaggtatgtctgttttgccataaatcagcagagtcatttcattctgggtgatcctaacacaccattgctatgttagatttgaaatgacatctctgttaaaagaatcttctatggaaataatggtgccctgcaaaatcttcctctgaactcacaggttagggatcacacaacttacttaatcgttttttgtttttgttttttttccttatatgtcaatggcccatgtcctccgggaaaattagaaaagcaaaatgattacaaagtgctgttagatttcttgtgctgggccagccaagtagaagtggacttgacttggacctttaactattttattacagattggacatttgctgttcagatgttttttaacagagggattatctcagaatcctgtgacctccaggttgttttataatctatttttctctatttaacattcctcagatagataggcaaataggacattccttctgtgtcacagaagtatcgtggtagtggcagtctacagtttatatgattcattgtaactatgagataaagaacaaccagtcatgtggccaaaaggattagatttgatttgatgttcacttggagtttactttttgtacatacaagataaaataaatattggatttgtaaaat

SEQ ID NO: 5 - protein, 1738 aa, Homo sapiens
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 6 - DNA, 453 bp, Homo sapiens
  allele 105: 99-62617-105  polymorphic base G or C
tgaggatgaggggcaaacacagaggaagaatctgggccccatgaggtcaacagtgagctgctaaaattaacctgaaatttccctgctgggagacattgtgttgtsaaatagttttataccatcttttactaggttttcaattaccagcagttggatgcaccctgtgatccaatggatacaaatgagatttagaagtgcactttaacttaaataaaataaatagccttgtctaatgtattaacgagaaagacatttttgtcttatttatatgtcttatgttctttgaaatgaaataaatgaccatatatgcttgtaaagagagggagtctacagatatttggagaaaggacattctgggtggagagaacacagagtgcaaaggacttgaagttaaagtgcatccagcagtctcaaggaagagtaaaggggtccatgtgagtggaataaggtgag

SEQ ID NO: 7 - DNA, 18 bp, Artificial Sequence
  sequencing oligonucleotide PrimerPU
tgtaaaacgacggccagt

SEQ ID NO: 8 - DNA, 18 bp, Artificial Sequence
  sequencing oligonucleotide PrimerRP
caggaaacagctatgacc

(19) United States
(12) Patent Application Publication
(10) Pub. No.: US 2008/0184381 A1
(43) Pub. Date: Jul. 31, 2008

(54) SCHIZOPHRENIA-RELATED VOLTAGE-GATED ION CHANNEL GENE AND PROTEIN

(75) Inventors: Daniel Cohen, Le Vesinet (FR); Ilya Chumakov, Vaux-le Penil (FR); Anne-Marie Simon, Paris (FR); Hadi Abderrahim, Maisons-Alfort (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: SERONO GENETICS INSTITUTE S.A., Evry (FR)

(21) Appl. No.: 12/050,492

(22) Filed: Mar. 18, 2008

Related U.S. Application Data
(63) Continuation of application No. 10/433,580, filed on Nov. 10, 2003.
(60) Provisional application No. 60/251,317, filed on Dec. 5, 2000.

(51) Int. Cl. A01K 67/00; C07H 21/04; C40B 40/08; C12P 21/04; G01N 33/53; C12Q 1/68; C07K 16/00; C12N 15/00

(52) U.S. Cl. 800/8; 536/23.5; 506/17; 435/320.1; 530/350; 435/69.1; 435/6; 435/7.1

(57) ABSTRACT

The invention concerns the genomic DNA, cDNA, and polypeptide sequences of CanIon, a novel voltage gated ion channel protein. The invention also concerns biallelic markers of the CanIon gene. The CanIon gene may be used as a biological target for the treatment and diagnosis of schizophrenia, bipolar disorder, and other diseases and conditions.

### CROSS-REFERENCE TO RELATED APPLICATIONS

This application is a continuation of U.S. application Ser. No. 10/433,580, filed Nov. 10, 2003, which is the national stage of international application No. PCT/IB01/02798, filed Dec. 4, 2001, which claims the benefit of U.S. Provisional Application Ser. No. 60/251,317, filed Dec. 5, 2000, the disclosures of which are hereby incorporated by reference in their entireties, including all figures, tables and amino acid or nucleic acid sequences.

The Sequence Listing for this application is labeled “Seq-List.txt” which was created on Mar. 18, 2008 and is 498 KB. The entire contents of the sequence listing is incorporated herein by reference in its entirety.

### FIELD OF THE INVENTION

The present invention is directed to a voltage-gated ion channel gene and protein and its role in disease. The invention relates to polynucleotides encoding a CanIon polypeptide as well as the regulatory regions located at the 5′- and 3′-end of said coding region. The invention also concerns polypeptides encoded by the CanIon gene. The invention also provides methods for screening for modulators, e.g. antagonists, of the CanIon channel, and methods of using such modulators in the treatment or prevention of various disorders or conditions. The invention also deals with antibodies directed specifically against such polypeptides that are useful as diagnostic reagents. The invention further encompasses biallelic markers of the CanIon gene useful in genetic analysis.

### BACKGROUND OF THE INVENTION

Advances in the technological armamentarium available to basic and clinical investigators have enabled increasingly sophisticated studies of brain and nervous system function in health and disease. Numerous hypotheses both neurobiological and pharmacological have been advanced with respect to the neurochemical and genetic mechanisms involved in central nervous system (CNS) disorders, including psychiatric disorders and neurodegenerative diseases. However, CNS disorders have complex and poorly understood etiologies, as well as symptoms that are overlapping, poorly characterized, and difficult to measure. As a result, future treatment regimes and drug development efforts will be required to be more sophisticated and focused on multigenic causes, and will need new assays to segment disease populations, and provide more accurate diagnostic and prognostic information on patients suffering from CNS disorders.

CNS disorders can encompass a wide range of disorders, and a correspondingly wide range of genetic factors. Examples of CNS disorders include neurodegenerative disorders, psychotic disorders, mood disorders, autism, substance dependence and alcoholism, pain disorders, epilepsy, mental retardation, and other psychiatric diseases including cognitive, anxiety, eating, impulse-control, and personality disorders. Disorders can be defined using the Diagnosis and Statistical Manual of Mental Disorders fourth edition (DSM-IV) classification.

Even when considering just a small subset of CNS disorders, it is evident from the lack of adequate treatment for and understanding of the molecular basis of the disorders schizophrenia and bipolar disorder that new targets for therapeutic invention and improved methods of treatment are needed. For both schizophrenia and bipolar disorder, all of the currently known molecules used for their treatment have side effects and act only against the symptoms of the disease. There is thus a strong need for new molecules without associated side effects and directed against targets which are involved in the causal mechanisms of schizophrenia and bipolar disorder. Therefore, tools facilitating the discovery and characterization of these targets are necessary and useful.

Voltage Gated Ion Channels

Voltage gated ion channels are part of a large family of macromolecules whose functions include the control and maintenance of electric potential across cell membranes, secretion and signal transduction. These channel proteins are involved in the control of neurotransmitter release from neurons, and play an important role in the regulation of a variety of cellular functions, including membrane excitability, muscle contraction and synaptic transmission. The main alpha-subunits of Na+ channels and the alpha-1 subunits of the Ca+ channels consist of approximately 2000 amino acids and contain the ion conduction pathway. Biochemical analysis has revealed that the physiologically active ion channel is composed of several different subunits. There are two auxiliary subunits that copurify with the alpha subunit of Na+ channels, the beta-1 and beta-2 subunit. For Ca+ channels, additional subunits (alpha-2, beta, gamma and sigma) have been identified with modulatory action. The alpha-2 and beta-subunits appear to enhance the functional activity of the alpha-1 subunit of Ca+ channels. The alpha-subunits of K+ channels are associated with beta subunits in a 1:1 fashion resulting in a K+ channel complex exhibiting (alpha)₄(beta)₄ stoichiometry (Terlau et al., Naturwissenschaften 85:437-444 (1998)). The basic structure and examples of calcium and sodium ion channels are further discussed, e.g., in Williams, et al. Science 257:389-395 (1992); Mori, et al., Nature 350:398-402 (1991); and Koch, et al., J. Biol. Chem. 265 (29):17786-17791 (1990). A Ca+ and Na+ ion channel nucleic acid sequence from the rat sharing a high level of sequence homology with the CanIon channel is further described in Lee et al., FEBS Lett. 445 23 :236 (1999).

The alpha subunit shares sequence characteristics with all voltage-dependent cation channels, and exploits the same structural motif comprising a 6-helix bundle of potential membrane spanning domains. In both sodium and calcium channels, this motif is repeated 4 times within the sequence to give a 24-helix bundle. The amino acid sequences are highly conserved among species (e.g., human and Drosophila), and among different ion channels.

There are several tissue-specific pharmacologically and electrophysiologically distinct isoforms of calcium channels, coded for by separate genes in a multi-gene family. In skeletal muscle, each tightly-bound assembly of alpha, beta and gamma subunits associates with 4 others to form a pentameric macromolecule. For example, neuronal calcium channel alpha-1 subunits are the product of at least seven different genes named alpha-1 A to H. Immunocytochemical studies have shown differential distribution of alpha-1 calcium channel subunits. Alpha-1A and alpha-1B are expressed mainly in dendrites and presynaptic terminals, and alpha-1A is generally concentrated in a larger number of nerve terminals than is alpha-1B. In the rat and human neuromuscular junction, alpha-1A is localized presynaptically, while alpha-1B and alpha-1A are both present in axon-associated Schwann cells. Alpha-1E is localized mainly in cell bodies, in some cases in proximal dendrites, as well as in the distal branches of Purkinje cells. Alpha-1C and alpha-1D are localized in cell bodies and in proximal dendrites of central neurons.

Native calcium channels have been classified based on their pharmacological and/or electrophysiological properties. The classification of voltage dependent calcium channels divides them into high voltage-activated (HVA), including L-, N-, P- and Q-types; intermediate (IVA, R-type); and low voltage-activated (LVA, T-type). (Morena et al., Annals NY Acad. Sci. 102-117 (1999).

The principal subunits (alpha-1) belong to a gene family whose members can form functional channels by themselves when expressed in heterologous expression systems. (Zhang et al., Neuropharmacology 32 (11): 1075-1088 (1993), incorporated herein by reference). In native cells, alpha-1 subunits are expressed as multisubunit complexes with ancillary subunits which modify the functional properties of the alpha-1 subunit. In many cases, coexpression of auxiliary subunits affects the biophysical properties of the channels. Beta subunits in particular tend to have important effects on the alpha-1 subunits; beta subunits have been shown to alter activation properties, steady state inactivation, inactivation kinetics and peak current.

Much of the molecular diversity of channels is produced by the existence of multiple forms of alpha-1 subunits. For example, it has been shown that differently spliced forms of calcium channels are differentially expressed and have different sensitivities to phosphorylation by serine-threonine kinases (Hell et al., Annals N.Y. Acad. Sci. 747:282-293 (1994)). Mutations in ion channel genes have been shown to be involved in a wide range of diseases, including several central nervous system diseases. Examples of ion channel mutations causing a number of episodic disorders including periodic paralysis, episodic ataxia, migraine, long QT syndrome and paroxysmal dyskenesia are reviewed in Bulman et al., Hum. Mol. Gen. 6(10) 1679-1685 (1997). Several Ca⁺ channel mutation disorders, for example, are shown in Table A (from Moreno, supra).

TABLE A

| Disease | Calcium channel subunit | Model |
|---|---|---|
| Familial hemiplegic | Alpha-1A | Human |
| migraine | | |
| Episodic ataxia type 2 | Alpha-1A | Human |
| Spinocerebellar ataxia | Alpha-1A | Human |
| typ 6 | | |
| Tottering and Learner | Alpha-1A | Mouse |
| phenotypes | | |
| Lambert-Eaton syndrome | Alpha-1A, α-1B | Human |
| Hypokalemic periodic | Alpha-1S | Human |
| paralysis | | |
| Muscular dysgenesis | Alpha-1S | Mouse |
| Zucker diabetic fatty | α-1C; α-1D | Rat |
| phenotypes | | |
| Lethargic phenotype | Beta-4 | Mouse |
| Malignant hypothermia | Alpha-2/δ | Human |
| Stargazer | Gamma | Mouse |

Modulators of calcium and sodium channels are also commonly used in the treatment of various diseases and conditions. For example, calcium and/or sodium channel blockers have been shown to be useful for the treatment or prevention of one or more symptoms associated with various diseases or conditions such as various heart diseases and conditions (e.g., angina, arrythmias), hypertension, migraines, neurological effects of strokes, mania, neuroleptic-induced tardive dyskinesia, bipolar disorder, pain, epilepsy, and others.

It has been shown that significant functional differences in the nervous system exist between different ion channels. In addition, functional differences exist between different mutations in the same ion channel gene as well as between splice variants of the same ion channel. Thus, despite the implication of ion channels in CNS disease, it has been difficult to predict which ion channel may be an effective target for therapeutic intervention for a particular disease. One problem has been to provide an ion channel gene implicated in schizophrenia, bipolar disorder or diseases related thereto.

The present invention addresses these and other needs.

### SUMMARY OF THE INVENTION

The present invention pertains to nucleic acid molecules comprising the genomic sequence of a novel human gene which encodes a voltage-gated ion channel protein, called CanIon. The CanIon genomic sequence also comprises regulatory sequence located upstream (5′-end) and downstream (3′-end) of the transcribed portion of said gene, these regulatory sequences being also part of the invention.

The invention also provides the complete cDNA sequence encoding the CanIon protein, as well as the corresponding translation product.

Oligonucleotide probes or primers hybridizing specifically with a CanIon genomic or cDNA sequence are also part of the present invention, as well as DNA amplification and detection methods using said primers and probes.

A further object of the invention consists of recombinant vectors comprising any of the nucleic acid sequences described above, and, in particular, of recombinant vectors comprising a CanIon regulatory sequence or a sequence encoding a CanIon protein, as well as of cell hosts and transgenic non human animals comprising said nucleic acid sequences or recombinant vectors.

The invention also concerns biallelic markers of the CanIon gene and the use thereof.

Finally, the invention is directed to methods for the screening of substances or molecules that modulate the expression or activity of CanIon, as well as with methods for the screening of substances or molecules that interact with a CanIon polypeptide. Methods of using substances identified in these methods are also provided. For example, methods of treating or prevention diseases or conditions including schizophrenia or bipolar using CanIon channel antagonists are provided.

Accordingly, in one aspect, the present invention provides an isolated, purified, or recombinant polynucleotide comprising any of the nucleotide sequences shown as SEQ ID Nos 1 to 4 or 6, or a sequence complementary to any of these sequences.

In another aspect, the present invention provides an isolated, purified, or recombinant polynucleotide comprising a contiguous span of at least 50 nucleotides of SEQ ID No 4, wherein said polynucleotide encodes a biologically active CanIon polypeptide.

In another aspect, the present invention provides an isolated, purified, or recombinant polynucleotide which encodes a human CanIon polypeptide comprising the amino acid sequence of SEQ ID No 5 or a biologically active fragment thereof.

In one embodiment, any of the herein-described polynucleotides is attached to a solid support.

In another embodiment, the polynucleotide comprises a label.

In another aspect, the present invention provides an array of polynucleotides comprising at least one of the herein-described polynucleotides. In one embodiment, the array is addressable.

In another aspect, the present invention provides a recombinant vector comprising any of the herein-described polynucleotides, operably linked to a promoter.

In another aspect, the present invention provides a polynucleotide whose presence in a cell causes an alteration in the level of expression of the CanIon gene. In one embodiment, the polynucleotide is inserted into the CanIon gene, or into the CanIon genomic region. In one embodiment, the polynucleotide is inserted into the CanIon gene promoter. In one embodiment, the polynucleotide is inserted by homologous recombination, e.g. by replacing one or more elements of the endogenous CanIon promoter or enhancer region.

In another aspect, the present invention provides a host cell or non-human host animal comprising any of the herein-described recombinant vectors or polynucleotides.

In another aspect, the present invention provides a mammalian host cell or non-human host mammal comprising a CanIon gene disrupted by homologous recombination with a knock out vector. In one embodiment, the host cell comprises any of the herein-described polynucleotides.

In another aspect, the present invention provides an isolated, purified, or recombinant polypeptide comprising the amino acid sequence shown as SEQ ID No 5, or a biologically active fragment thereof.

In another aspect, the present invention provides a method of making a polypeptide, the method comprising a) providing a population of cells comprising a polynucleotide encoding the polypeptide of claim 13, operably linked to a promoter; b) culturing said population of cells under conditions conducive to the production of said polypeptide within said cells; and c) purifying said polypeptide from said population of cells.

In another aspect, the present invention provides a method of binding an anti-CanIon antibody to a CanIon polypeptide, comprising contacting said antibody with any of the herein-described CanIon polypeptides under conditions in which the antibody can specifically bind to said polypeptide. In another aspect, antibodies, or immunologically active fragments thereof, that specifically recognize a CanIon protein or epitope are also provided.

In another aspect, the present invention provides a method of detecting the expression of a CanIon gene within a cell, said method comprising the steps of: a) contacting said cell or an extract of said cell with either of: i) a polynucleotide that hybridizes under stringent conditions to any of the herein-described CanIon polynucleotides; or ii) a polypeptide that specifically binds to any of the herein-described CanIon polypeptides; and b) detecting the presence or absence of hybridization between said polynucleotide and an RNA species within said cell or extract, or the presence or absence of binding of said polypeptide to a protein within said cell or extract;

wherein a detection of the presence of said hybridization or of said binding indicates that said CanIon gene is expressed within said cell. In one embodiment, said polynucleotide is a primer, and said hybridization is detected by detecting the presence of an amplification product comprising the sequence of said primer. In another embodiment, said polypeptide is an antibody, e.g. an anti-CanIon antibody.

In another aspect, the present invention provides a method of identifying a candidate modulator of a CanIon polypeptide, said method comprising: a) contacting any of the herein-described CanIon polypeptides with a test compound; and b) determining whether said compound specifically binds to said polypeptide; wherein a detection that said compound specifically binds to said polypeptide indicates that said compound is a candidate modulator of said CanIon polypeptide.

In one embodiment, the method further comprises testing the biological activity of said CanIon polypeptide in the presence of said candidate modulator, wherein an alteration in the biological activity of said CanIon polypeptide in the presence of said candidate modulator in comparison to the activity in the absence of said candidate modulator indicates that the candidate modulator is a modulator of said CanIon polypeptide.

In another aspect, the present invention provides a method of identifying a modulator of a CanIon polypeptide, said method comprising: a) contacting any of the herein-described CanIon polypeptides with a test compound; and b) detecting the activity of said polypeptide in the presence and absence of said compound; wherein a detection of a difference in said activity in the presence of said compound in comparison to the activity in the absence of said compound indicates that said compound is a modulator of said CanIon polypeptide.

In one embodiment of the present methods, said polypeptide is present in a cell or cell membrane, and said biological activity comprises voltage gated ion channel activity.

In another aspect, the present invention provides a method for the preparation of a pharmaceutical composition comprising a) identifying a modulator of a CanIon polypeptide using any of the herein-described methods; and b) combining said modulator with a physiologically acceptable carrier. Methods of using the pharmaceutical compositions are also provided.

Uses of any of the presently-described CanIon modulators, polypeptides, polynucleotides, or antibodies in the preparation of a medicament, e.g. for the treatment of the human body or for the treatment of any of the herein-described diseases or conditions, are also provided.

Kits for using and detecting the present CanIon polynucleotides and polypeptides in vitro or in vivo are also provided.

### BRIEF DESCRIPTION OF THE DRAWINGS

FIG. 1 is a diagram showing a BAC map of the chromosome 13q region containing the CanIon gene.

FIG. 2 is a block diagram of an exemplary computer system.

FIG. 3 is a flow diagram illustrating one embodiment of a process 200 for comparing a new nucleotide or protein sequence with a database of sequences in order to determine the homology levels between the new sequence and the sequences in the database.

FIG. 4 is a flow diagram illustrating one embodiment of a process 250 in a computer for determining whether two sequences are homologous.

FIG. 5 is a flow diagram illustrating one embodiment of an identifier process 300 for detecting the presence of a feature in a sequence.

### BRIEF DESCRIPTION OF THE SEQUENCES PROVIDED IN THE SEQUENCE LISTING

SEQ ID No 1 contains a genomic sequence of CanIon comprising the 5′ regulatory region (upstream untranscribed region) and exons 1 to 7.

SEQ ID No 2 contains a genomic sequence of CanIon comprising exons 8 to 27.

SEQ ID No 3 contains a genomic sequence of CanIon comprising exons 28 to 44, and the 3′ regulatory region (downstream untranscribed region).

SEQ ID No 4 contains a cDNA sequence of CanIon.

SEQ ID No 5 contains the amino acid sequence encoded by the cDNA of SEQ ID No 4.

SEQ ID No 6 contains the nucleotide sequence of the amplicon which comprises biallelic marker A18.

SEQ ID No 7 contains a primer containing the additional PU 5′ sequence described further in Example 2

SEQ ID No 8 contains a primer containing the additional RP 5′ sequence described further in Example 2.

In accordance with the regulations relating to Sequence Listings, the following codes have been used in the Sequence Listing to indicate the locations of biallelic markers within the sequences and to identify each of the alleles present at the polymorphic base. The code “r” in the sequences indicates that one allele of the polymorphic base is a guanine, while the other allele is an adenine. The code “y” in the sequences indicates that one allele of the polymorphic base is a thymine, while the other allele is a cytosine. The code “m” in the sequences indicates that one allele of the polymorphic base is an adenine, while the other allele is an cytosine. The code “k” in the sequences indicates that one allele of the polymorphic base is a guanine, while the other allele is a thymine. The code “s” in the sequences indicates that one allele of the polymorphic base is a guanine, while the other allele is a cytosine. The code “w” in the sequences indicates that one allele of the polymorphic base is an adenine, while the other allele is an thymine. The nucleotide code of the original allele for each biallelic marker is the following:

| Biallelic marker | Original allele |
|---|---|
| 5-124-273 | A (for example) |

In some instances, the polymorphic bases of the biallelic markers alter the identity of an amino acids in the encoded polypeptide. This is indicated in the accompanying Sequence Listing by use of the feature VARIANT, placement of an Xaa at the position of the polymorphic amino acid, and definition of Xaa as the two alternative amino acids. For example if one allele of a biallelic marker is the codon CAC, which encodes histidine, while the other allele of the biallelic marker is CAA, which encodes glutamine, the Sequence Listing for the encoded polypeptide will contain an Xaa at the location of the polymorphic amino acid. In this instance, Xaa would be defined as being histidine or glutamine.

In other instances, Xaa may indicate an amino acid whose identity is unknown because of nucleotide sequence ambiguity. In this instance, the feature UNSURE is used, placement of an Xaa at the position of the unknown amino acid and definition of Xaa as being any of the 20 amino acids or a limited number of amino acids suggested by the genetic code.

### DETAILED DESCRIPTION

The aggregation of schizophrenia and bipolar disorder in families, the evidence from twin and adoption studies, and the lack of variation in incidence worldwide, indicate that schizophrenia and bipolar disorder are primarily genetic conditions, although environmental risk factors are also involved at some level as necessary, sufficient, or interactive causes. For example, schizophrenia occurs in 1% of the general population. But, if there is one grandparent with schizophrenia, the risk of getting the illness increases to about 3%; if there is one parent with schizophrenia the risk rises to about 10%. When both parents have schizophrenia, the risk rises to approximately 40%.

The identification of genes involved in a particular trait such as a specific central nervous system disorder, like schizophrenia, can be carried out through two main strategies currently used for genetic mapping: linkage analysis and association studies. Linkage analysis requires the study of families with multiple affected individuals and is now useful in the detection of mono- or oligogenic inherited traits. Conversely, association studies examine the frequency of marker alleles in unrelated trait (T+) individuals compared with trait negative (T−) controls, and are generally employed in the detection of polygenic inheritance.

Genetic link or “linkage” is based on an analysis of which of two neighboring sequences on a chromosome contains the least recombinations by crossing-over during meiosis. To do this, chromosomal markers, like microsatellite markers, have been localized with precision on the genome. Genetic linkage analysis calculates the probabilities of recombinations on the target gene with the chromosomal markers used, according to the genealogical tree, the transmission of the disease, and the transmission of the markers. Thus, if a particular allele of a given marker is transmitted with the disease more often than chance would have it (recombination level between 0 and 0.5), it is possible to deduce that the target gene in question is found in the neighborhood of the marker. Using this technique, it has been possible to localize several genes demonstrating a genetic predisposition of familial cancers. In order to be able to be included in a genetic linkage study, the families affected by a hereditary form of the disease must satisfy the “informativeness” criteria: several affected subjects (and whose constitutional DNA is available) per generation, and at best having a large number of siblings.

Results of linkage studies supported the hypothesis that chromosome 13 was likely to harbor a schizophrenia susceptibility locus on 13q32 (Blouin J L et al., 1998, Nature Genetics, 20:70-73; Lin M W et al., Hum. Genet., 99(3) (1997):417-420; Brzustowicz et al., Am. J. Hum. Genet. 65:1096-1103 (1999)). However, while linkage analysis is a powerful method for detecting genes involved in a trait, resolution is often not possible beyond the megabase level and complementary studies are often required to refine the analysis of the regions initially identified through this method.

A BAC contig covering a candidate genomic region of the chromosome 13q-31-q33 locus was constructed using public STSs localised in the chromosome 13q31-q33 region to screen a 7 genome equivalent proprietary BAC library. From these materials, new STSs were generated allowing construction of a dense physical map of the region. BACs were all sized and mapped by in situ chromosomal hybridisation for verification. A minimal set of BACs was identified and fully sequenced which resulted in several contigs leading to the eventual construction of a contig of over 4 Mb. The construction of this map led to the identification of the CanIon gene which is located within a genomic region showing significant linkage to schizophrenia.

The CanIon amino acid sequence is characteristic of CACHANNEL, a 7-element fingerprint that provides a signature for the alpha-1 subunit of calcium channels (Ref. PRO0167, BLOCKs+ database). The fingerprint was derived from an initial alignment of 6 sequences: the motifs were drawn from conserved loop regions capable of distinguishing between these and other cation channels; motifs 1 and 2 encode those between transmembrane segments 4 and 5, and 5 and 6 (first internal repeat); motif 3 corresponds to that between segment 6 of repeat 1 and segment 1 of repeat 2; motif 4 encodes that between segments 5 and 6 of repeat 2; motif 5 corresponds to that between segment 6 of repeat 3 and segment 1 of repeat 4; and motifs 6 and 7 encode those between segments 4 and 5, and 5 and 6 of repeat 4.

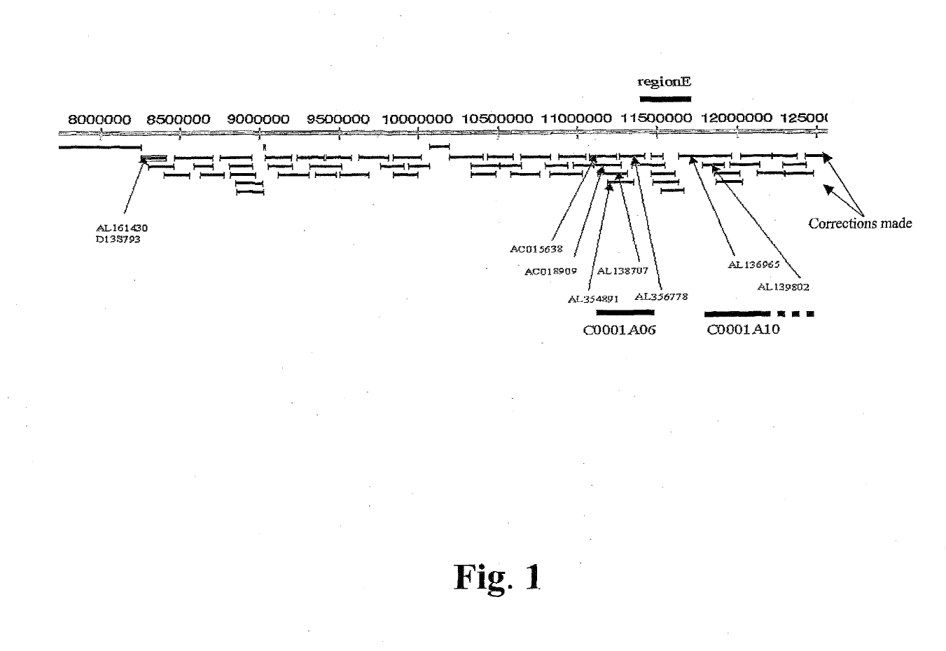

FIG. 1 shows BAC contigs covering a chromosome 13 region of interest which includes the CanIon gene, and shows the genomic location of the CanIon gene in relation to genetic markers showing the highest significance in linkage studies. In particular, Blouin et al. (1998) conducted a genome wide scan for schizophrenia susceptibility loci using 452 microsatellite markers on 54 complex pedigrees. The most significant linkage between schizophrenia in families was found on chromosome 13q32 near marker D13S174. Brzustowics et al. (1999) evaluated microsatellite markers spanning chromosomes 8 and 13 in 21 extended Canadian families. Markers in the chromosome 13q region produced positive LOD scores in each analysis model used: autosomal dominant and recessive, with narrow or broad definition of schizophrenia. Maximum three point LOD scores were obtained with marker D13S793 under a recessive-broad model: 3.92 at recombinant fraction (θ) 0.1 under homogeneity and 4.42 with α=0.65 and θ=0 under heterogeneity. Referring to FIG. 1, the CanIon gene is located partly on the contig labelled ‘Region E’ and partly on the contig labelled C0001A10. The CanIon gene is flanked by the two markers showing highest significance in linkage studies. Marker D13S174 is also on contig C0001A10, while marker D13S793 is located approximately 3.5 Mb centromeric to the CanIon gene.

There is a strong need to identify genes involved in schizophrenia, bipolar disorder, and other CNS and cardiovascular diseases and conditions. There is also a need to identify novel ion channels involved in diseases. These genes and proteins may provide new intervention points in the treatment of schizophrenia, bipolar disorder, or other CNS conditions, as well as other conditions such as heart conditions and hypertension, and allow further study and characterization of the CanIon gene and its related biological pathway. The knowledge of these genes and the related biological pathways involved in these diseases and conditions will allow researchers to understand the etiology of, e.g., schizophrenia and bipolar disorder and will lead to drugs and medications which are directed against the cause of the diseases. For example, compounds that block CanIon channels can be used to treat any of a number of diseases or conditions, preferably schizophrenia or bipolar disorder, and also including pain disorders, epilepsy, and various cardiovascular disorders such as heart arrythmias, angina, and hypertension. There is also a great need for new methods of detecting a susceptibility to schizophrenia, bipolar disorder, and other conditions, as well as for preventing or following up the development of any of these diseases. Diagnostic tools could also prove extremely useful. Indeed, to give one example, early identification of subjects at risk of developing schizophrenia would enable early and/or prophylactic treatment to be administered. Moreover, accurate assessments of the efficacy of a medicament as well as the patient's tolerance to it may enable clinicians to enhance the benefit/risk ratio of schizophrenia and bipolar disorder treatment regimes.

The present invention concerns polynucleotides and polypeptides related to the CanIon gene. Oligonucleotide probes and primers hybridizing specifically with a genomic or a cDNA sequence of CanIon are also part of the invention. A further object of the invention consists of recombinant vectors comprising any of the nucleic acid sequences described in the present invention, and in particular recombinant vectors comprising a regulatory region of CanIon or a sequence encoding the CanIon protein, as well as cell hosts comprising said nucleic acid sequences or recombinant vectors. The invention also encompasses methods of screening molecules for the ability to modulate the expression or activity of the CanIon gene or protein, as well as methods of using such molecules for the treatment or prevention of schizophrenia, bipolar disorder, or any of a number of other diseases or conditions. The invention also deals with antibodies directed specifically against such polypeptides that are useful as diagnostic reagents.

The invention also concerns CanIon-related biallelic markers and their use in methods of genetic analysis including linkage studies in families, linkage disequilibrium studies in populations and association studies in case-control populations. An important aspect of the present invention is that biallelic markers allow association studies to be performed to identify the role of genes involved in complex traits.

### DEFINITIONS

Before describing the invention in greater detail, the following definitions are set forth to illustrate and define the meaning and scope of the terms used to describe the invention herein.

The terms “CanIon gene”, when used herein, encompasses genomic, mRNA and cDNA sequences encoding the CanIon protein, including the untranslated regulatory regions of the genomic DNA.

The term “heterologous protein”, when used herein, is intended to designate any protein or polypeptide other than the CanIon protein. For example, the heterologous protein may be a compound which can be used as a marker in further experiments with a CanIon regulatory region.

The term “isolated” requires that the material be removed from its original environment (e.g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

For example, a naturally-occurring polynucleotide present in a living animal is not isolated, but the same polynucleotide, separated from some or all of the coexisting materials in the natural system, is isolated. Specifically excluded from the definition of “isolated” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified polynucleotide makes up less than 5% of the number of nucleic acid inserts in the vector molecules. Further specifically excluded are whole cell genomic DNA or whole cell RNA preparations (including said whole cell preparations which are mechanically sheared or enzymatically digested). Further specifically excluded are the above whole cell preparations as either an in vitro preparation or as a heterogeneous mixture separated by electrophoresis (including blot transfers of the same) wherein the polynucleotide of the invention has not further been separated from the heterologous polynucleotides in the electrophoresis medium (e.g., further separating by excising a single band from a heterogeneous band population in an agarose gel or nylon blot).

The term “purified” does not require absolute purity; rather, it is intended as a relative definition. Purification of starting material or natural material to at least one order of magnitude, preferably two or three orders, and more preferably four or five orders of magnitude is expressly contemplated. As an example, purification from 0.1% concentration to 10% concentration is two orders of magnitude. To illustrate, individual cDNA clones isolated from a cDNA library have been conventionally purified to electrophoretic homogeneity. The sequences obtained from these clones could not be obtained directly either from the library or from total human DNA. The cDNA clones are not naturally occurring as such, but rather are obtained via manipulation of a partially purified naturally occurring substance (messenger RNA). The conversion of mRNA into a cDNA library involves the creation of a synthetic substance (cDNA) and pure individual cDNA clones can be isolated from the synthetic library by clonal selection. Thus, creating a cDNA library from messenger RNA and subsequently isolating individual clones from that library results in an approximately 10⁴-10⁶ fold purification of the native message.

The term “purified” is further used herein to describe a polypeptide or polynucleotide of the invention which has been separated from other compounds including, but not limited to, polypeptides or polynucleotides, carbohydrates, lipids, etc. The term “purified” may be used to specify the separation of monomeric polypeptides of the invention from oligomeric forms such as homo- or hetero-dimers, trimers, etc. The term “purified” may also be used to specify the separation of covalently closed polynucleotides from linear polynucleotides. A polynucleotide is substantially pure when at least about 50%, preferably 60 to 75% of a sample exhibits a single polynucleotide sequence and conformation (e.g., linear versus covalently closed). A substantially pure polypeptide or polynucleotide typically comprises about 50%, preferably 60 to 90% weight/weight of a polypeptide or polynucleotide sample, respectively, more usually about 95%, and preferably is over about 99% pure. Polypeptide and polynucleotide purity, or homogeneity, is indicated by a number of means well known in the art, such as agarose or polyacrylamide gel electrophoresis of a sample, followed by visualizing a single band upon staining the gel. For certain purposes higher resolution can be provided by using HPLC or other means well known in the art. As an alternative embodiment, purification of the polypeptides and polynucleotides of the present invention may be expressed as “at least” a percent purity relative to heterologous polypeptides and polynucleotides (DNA, RNA or both). As a preferred embodiment, the polypeptides and polynucleotides of the present invention are at least; 10%, 20%, 30%, 40%, 50%, 60%, 70%, 80%, 90%, 95%, 96%, 96%, 98%, 99%, or 100% pure relative to heterologous polypeptides and polynucleotides, respectively. As a further preferred embodiment the polypeptides and polynucleotides have a purity ranging from any number, to the thousandth position, between 90% and 100% (e.g., a polypeptide or polynucleotide at least 99.995% pure) relative to either heterologous polypeptides or polynucleotides, respectively, or as a weight/weight ratio relative to all compounds and molecules other than those existing in the carrier. Each number representing a percent purity, to the thousandth position, may be claimed as individual species of purity.

The term “polypeptide” refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

As used herein, the term “non-human animal” refers to any non-human vertebrate, birds and more usually mammals, preferably primates, farm animals such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)₂, and F(ab′)₂ fragments.

As used herein, an “antigenic determinant” is the portion of an antigen molecule, in this case a CanIon polypeptide, that determines the specificity of the antigen-antibody reaction. An “epitope” refers to an antigenic determinant of a polypeptide. An epitope can comprise as few as 3 amino acids in a spatial conformation which is unique to the epitope. Generally an epitope comprises at least 6 such amino acids, and more usually at least 8-10 such amino acids. Methods for determining the amino acids which make up an epitope include x-ray crystallography, 2-dimensional nuclear magnetic resonance, and epitope mapping e.g. the Pepscan method described by Geysen et al. 1984; PCT Publication No. WO 84/03564; and PCT Publication No. WO 84/03506.

Throughout the present specification, the expression “nucleotide sequence” may be employed to designate indifferently a polynucleotide or a nucleic acid. More precisely, the expression “nucleotide sequence” encompasses the nucleic material itself and is thus not restricted to the sequence information (i.e. the succession of letters chosen among the four base letters) that biochemically characterizes a specific DNA or RNA molecule.

As used interchangeably herein, the terms “nucleic acids”, “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, for examples of analogous linking groups, purine, pyrimidines, and sugars see for example PCT publication No. WO 95/04064. The polynucleotide sequences of the invention may be prepared by any known method, including synthetic, recombinant, ex vivo generation, or a combination thereof, as well as utilizing any purification methods known in the art.

A sequence which is “operably linked” to a regulatory sequence such as a promoter means that said regulatory element is in the correct location and orientation in relation to the nucleic acid to control RNA polymerase initiation and expression of the nucleic acid of interest. As used herein, the term “operably linked” refers to a linkage of polynucleotide elements in a functional relationship. For instance, a promoter or enhancer is operably linked to a coding sequence if it affects the transcription of the coding sequence.

The terms “trait” and “phenotype” are used interchangeably herein and refer to any visible, detectable or otherwise measurable property of an organism such as symptoms of, or susceptibility to, a disease for example. Typically the terms “trait” or “phenotype” are used herein to refer to symptoms of, or susceptibility to a disease, a beneficial response to or side effects related to a treatment. Preferably, said trait can be, without being limited to, psychiatric disorders such as schizophrenia or bipolar disorder, other CNS or neuronal disorders such as epilepsy or pain disorders, as well as cardiovascular conditions such as anginas, hypertension, and arrythmias, as well as any aspect, feature, or characteristic of any of these diseases or conditions.

The term “allele” is used herein to refer to variants of a nucleotide sequence. A biallelic polymorphism has two forms. Diploid organisms may be homozygous or heterozygous for an allelic form.

The term “heterozygosity rate” is used herein to refer to the incidence of individuals in a population which are heterozygous at a particular allele. In a biallelic system, the heterozygosity rate is on average equal to 2Pₐ(1−Pₐ), where Pₐ is the frequency of the least common allele. In order to be useful in genetic studies, a genetic marker should have an adequate level of heterozygosity to allow a reasonable probability that a randomly selected person will be heterozygous.

The term “genotype” as used herein refers to the identity of the alleles present in an individual or a sample. In the context of the present invention, a genotype preferably refers to the description of the biallelic marker alleles present in an individual or a sample, e.g. the alleles of biallelic markers within the CanIon gene or genomic region. The term “genotyping” a sample or an individual for a biallelic marker involves determining the specific allele or the specific nucleotide carried by an individual at a biallelic marker.

The term “mutation” as used herein refers to a difference in DNA sequence between or among different genomes or individuals which has a frequency below 1%.

The term “haplotype” refers to a combination of alleles present in an individual or a sample. In the context of the present invention, a haplotype preferably refers to a combination of biallelic marker alleles found in a given individual and which may be associated with a phenotype.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is the replacement of one nucleotide by another nucleotide at the polymorphic site. Deletion of a single nucleotide or insertion of a single nucleotide also gives rise to single nucleotide polymorphisms. In the context of the present invention, “single nucleotide polymorphism” preferably refers to a single nucleotide substitution. Typically, between different individuals, the polymorphic site may be occupied by two different nucleotides.

The term “biallelic polymorphism” and “biallelic marker” are used interchangeably herein to refer to a single nucleotide polymorphism having two alleles at a fairly high frequency in the population. A “biallelic marker allele” refers to the nucleotide variants present at a biallelic marker site. Typically, the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A biallelic marker wherein the frequency of the less common allele is 30% or more is termed a “high quality biallelic marker”.

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center”, and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.”If the difference is 0 to 5, the polymorphism is considered to be “within 2 nucleotides of the center.” If the difference is 0 to 7, the polymorphism is considered to be “within 3 nucleotides of the center,” and so on.

The term “upstream” is used herein to refer to a location which is toward the 5′ end of the polynucleotide from a specific reference point, or, in the case of a gene, in the direction running from the coding sequence to the promoter.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4^{th} edition, 1995).

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind.

Variants and Fragments

1—Polynucleotides

The invention also relates to variants and fragments of the polynucleotides described herein, particularly of a CanIon gene containing one or more biallelic markers according to the invention.

Variants of polynucleotides, as the term is used herein, are polynucleotides that differ from a reference polynucleotide. A variant of a polynucleotide may be a naturally occurring variant such as a naturally occurring allelic variant, or it may be a variant that is not known to occur naturally. Such non-naturally occurring variants of the polynucleotide may be made by mutagenesis techniques, including those applied to polynucleotides, cells or organisms. Generally, differences are limited so that the nucleotide sequences of the reference and the variant are closely similar overall and, in many regions, identical.

Variants of polynucleotides according to the invention include, without being limited to, nucleotide sequences which are at least 95% identical to a polynucleotide selected from the group consisting of the nucleotide sequences of SEQ ID Nos 1 to 4 or to any polynucleotide fragment of at least 12 consecutive nucleotides of a polynucleotide selected from the group consisting of the nucleotide sequences of SEQ ID Nos 1 to 4, and preferably at least 99% identical, more particularly at least 99.5% identical, and most preferably at least 99.8% identical to a polynucleotide selected from the group consisting of the nucleotide sequences of SEQ ID Nos 1 to 4 or to any polynucleotide fragment of at least 12 consecutive nucleotides of a polynucleotide selected from the group consisting of the nucleotide sequences of SEQ ID No 1 to 4.

Nucleotide changes present in a variant polynucleotide may be silent, which means that they do not alter the amino acids encoded by the polynucleotide. However, nucleotide changes may also result in amino acid substitutions, additions, deletions, fusions and truncations in the polypeptide encoded by the reference sequence. The substitutions, deletions or additions may involve one or more nucleotides. The variants may be altered in coding or non-coding regions or both. Alterations in the coding regions may produce conservative or non-conservative amino acid substitutions, deletions or additions.

In the context of the present invention, particularly preferred embodiments are those in which the polynucleotides encode polypeptides which retain substantially the same biological function or activity as the mature CanIon protein, or those in which the polynucleotides encode polypeptides which maintain or increase a particular biological activity, while reducing a second biological activity

A polynucleotide fragment is a polynucleotide having a sequence that is entirely the same as part but not all of a given nucleotide sequence, preferably the nucleotide sequence of a CanIon gene, and variants thereof. The fragment can be a portion of an intron or an exon of a CanIon gene. It can also be a portion of the regulatory regions of CanIon. Preferably, such fragments comprise at least one of the biallelic markers A1 to A17 or the complements thereto or a biallelic marker in linkage disequilibrium with one or more of the biallelic markers A1 to A17.

Such fragments may be “free-standing”, i.e. not part of or fused to other polynucleotides, or they may be comprised within a single larger polynucleotide of which they form a part or region. Indeed, several of these fragments may be present within a single larger polynucleotide.

Optionally, such fragments may consist of, or consist essentially of, a contiguous span of at least 8, 10, 12, 15, 18, 20, 25, 35, 40, 50, 70, 80, 100, 250, 500 or 1000 nucleotides in length. A set of preferred fragments contain at least one of the biallelic markers A1 to A17 of the CanIon gene which are described herein or the complements thereto.

2—Polypeptides

The invention also relates to variants, fragments, analogs and derivatives of the polypeptides described herein, including mutated CanIon proteins.

The variant may be 1) one in which one or more of the amino acid residues are substituted with a conserved or non-conserved amino acid residue and such substituted amino acid residue may or may not be one encoded by the genetic code, or 2) one in which one or more of the amino acid residues includes a substituent group, or 3) one in which the mutated CanIon is fused with another compound, such as a compound to increase the half-life of the polypeptide (for example, polyethylene glycol), or 4) one in which the additional amino acids are fused to the mutated CanIon, such as a leader or secretory sequence or a sequence which is employed for purification of the mutated CanIon or a preprotein sequence. Such variants are deemed to be within the scope of those skilled in the art.

A polypeptide fragment is a polypeptide having a sequence that entirely is the same as part but not all of a given polypeptide sequence, preferably a polypeptide encoded by a CanIon gene and variants thereof.

In the case of an amino acid substitution in the amino acid sequence of a polypeptide according to the invention, one or several amino acids can be replaced by “equivalent” amino acids. The expression “equivalent” amino acid is used herein to designate any amino acid that may be substituted for one of the amino acids having similar properties, such that one skilled in the art of peptide chemistry would expect the secondary structure and hydropathic nature of the polypeptide to be substantially unchanged. Generally, the following groups of amino acids represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

A specific embodiment of a modified CanIon peptide molecule of interest according to the present invention includes, but is not limited to, a peptide molecule which is resistant to proteolysis, is a peptide in which the —CONH— peptide bond is modified and replaced by a (CH2NH) reduced bond, a (NHCO) retro inverso bond, a (CH2—O) methylene-oxy bond, a (CH2—S) thiomethylene bond, a (CH2CH2) carba bond, a (CO—CH2) cetomethylene bond, a (CHOH—CH2) hydroxyethylene bond), a (N—N) bound, a E-alcene bond or also a —CH═CH— bond. The invention also encompasses a human CanIon polypeptide or a fragment or a variant thereof in which at least one peptide bond has been modified as described above.

Such fragments may be “free-standing”, i.e. not part of or fused to other polypeptides, or they may be comprised within a single larger polypeptide of which they form a part or region. However, several fragments may be comprised within a single larger polypeptide.

As representative examples of polypeptide fragments of the invention, there may be mentioned those which have from about 5, 6, 7, 8, 9 or 10 to 15, 10 to 20, 15 to 40, or 30 to 55 amino acids long. Preferred are those fragments containing at least one amino acid mutation in the CanIon protein.

Identity Between Nucleic Acids or Polypeptides

The terms “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Homology is evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, 1988; Altschul et al., 1990; Thompson et al., 1994; Higgins et al., 1996; Altschul et al., 1990; Altschul et al., 1993). In a particularly preferred embodiment, protein and nucleic acid sequence homologies are evaluated using the Basic Local Alignment Search Tool (“BLAST”) which is well known in the art (see, e.g., Karlin and Altschul, 1990; Altschul et al., 1990, 1993, 1997). In particular, five specific BLAST programs are used to perform the following task:

(1) BLASTP and BLAST3 compare an amino acid query sequence against a protein sequence database;

(2) BLASTN compares a nucleotide query sequence against a nucleotide sequence database;

(3) BLASTX compares the six-frame conceptual translation products of a query nucleotide sequence (both strands) against a protein sequence database;

(4) TBLASTN compares a query protein sequence against a nucleotide sequence database translated in all six reading frames (both strands); and

(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., 1992; Henikoff and Henikoff, 1993). Less preferably, the PAM or PAM250 matrices may also be used (see, e.g., Schwartz and Dayhoff, eds., 1978). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (see, e.g., Karlin and Altschul, 1990). The BLAST programs may be used with the default parameters or with modified parameters provided by the user.

Stringent Hybridization Conditions

For the purpose of defining such a hybridizing nucleic acid according to the invention, the stringent hybridization conditions are the following:

the hybridization step is carried out at 65° C. in the presence of 6×SSC buffer, 5×Denhardt's solution, 0.5% SDS and 100 μg/ml of salmon sperm DNA.

The hybridization step is followed by four washing steps:
two washings of 5 min, preferably at 65° C. in a 2×SSC and 0.1% SDS buffer;
one washing of 30 min, preferably at 65° C. in a 2×SSC and 0.1% SDS buffer,
one washing of 10 min, preferably at 65° C. in a 0.1×SSC and 0.1% SDS buffer,

these hybridization conditions being suitable for a nucleic acid molecule of about 20 nucleotides in length. There is no need to say that the hybridization conditions described above are to be adapted according to the length of the desired nucleic acid, following techniques well known to the one skilled in the art. The suitable hybridization conditions may for example be adapted according to the teachings disclosed in the book of Hames and Higgins (1985).

### Genomic Sequences of the CanIon Gene

The present invention concerns the genomic sequence of CanIon. The present invention encompasses the CanIon gene, or CanIon genomic sequences consisting of, consisting essentially of, or comprising the sequence of SEQ ID Nos 1 to 3, a sequence complementary thereto, as well as fragments and variants thereof. These polynucleotides may be purified, isolated, or recombinant.

The invention also encompasses a purified, isolated, or recombinant polynucleotide comprising a nucleotide sequence having at least 70, 75, 80, 85, 90, or 95% nucleotide identity with a nucleotide sequence of SEQ ID Nos 1 to 3 or a complementary sequence thereto or a fragment thereof. The nucleotide differences as regards to the nucleotide sequence of SEQ ID Nos 1 to 3 may be generally randomly distributed throughout the entire nucleic acid. Nevertheless, preferred nucleic acids are those wherein the nucleotide differences as regards to the nucleotide sequence of SEQ ID Nos 1 to 3 are predominantly located outside the coding sequences contained in the exons. These nucleic acids, as well as their fragments and variants, may be used as oligonucleotide primers or probes in order to detect the presence of a copy of the CanIon gene in a test sample, or alternatively in order to amplify a target nucleotide sequence within the CanIon sequences.

Another object of the invention consists of a purified, isolated, or recombinant nucleic acid that hybridizes with the nucleotide sequence of SEQ ID Nos 1 to 3 or a complementary sequence thereto or a variant thereof, under the stringent hybridization conditions as defined above. In preferred embodiments, said purified, isolated, or recombinant nucleic acid hybridizes specifically with the polynucleotides of the human CanIon gene, more preferably said nucleic acid is capable of hybridizing to the nucleotides of the human CanIon gene but is substantially incapable of hybridizing to nucleic sequence of the rat CanIon gene.

Particularly preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, 1000, 2000, 3000, 4000, 5000 or 10000 nucleotides of SEQ ID No 1 to 3 or the complements thereof. It should be noted that nucleic acid fragments of any size and sequence may also be comprised by the polynucleotides described in this section.

The CanIon genomic nucleic acid comprises 44 exons. The exon positions in SEQ ID No 1 to 3 are detailed below in Table B.

TABLE B

| | Position in | | | Position in | |
|---|---|---|---|---|---|
| | SEQ ID No 1 | | | SEQ ID No 1 | |
| Exon | Beginning | End | Intron | Beginning | End |
| 1 | 2001 | 2026 | 1 | 2027 | 19187 |
| 2 | 19188 | 19334 | 2 | 19335 | 22995 |
| 3 | 22996 | 23178 | 3 | 23179 | 39730 |
| 4 | 39731 | 39814 | 4 | 39815 | 41336 |
| 5 | 41337 | 41476 | 5 | 41477 | 41564 |
| 6 | 41565 | 41693 | 6 | 41694 | 73012 |
| 7 | 73013 | 73167 | | | |
| | Position in | | | Position in | |
| | SEQ ID No 2 | | | SEQ ID No 2 | |
| Exon | Beginning | End | Intron | Beginning | End |
| 8 | 43726 | 43868 | 7 | 43869 | 43997 |
| 9 | 43998 | 44102 | 8 | 44103 | 52092 |
| 10 | 52093 | 52179 | 9 | 52180 | 77567 |
| 11 | 77568 | 77699 | 10 | 77700 | 98225 |
| 12 | 98226 | 98393 | 11 | 98394 | 106566 |
| 13 | 106567 | 106758 | 12 | 106759 | 144108 |
| 14 | 144109 | 144246 | 13 | 144247 | 159793 |
| 15 | 159794 | 159868 | 14 | 159869 | 191291 |
| 16 | 191292 | 191428 | 15 | 191429 | 192966 |
| 17 | 192967 | 193108 | 16 | 193109 | 211539 |
| 18 | 211540 | 211613 | 17 | 211614 | 225005 |
| 19 | 225006 | 225107 | 18 | 225108 | 225543 |
| 20 | 225544 | 225613 | 19 | 225614 | 228449 |
| 21 | 228450 | 228541 | 20 | 228542 | 228629 |
| 22 | 228630 | 228752 | 21 | 228753 | 231288 |
| 23 | 231289 | 231345 | 22 | 231346 | 231588 |
| 24 | 231589 | 231709 | 23 | 231710 | 231812 |
| 25 | 231813 | 231944 | 24 | 231945 | 232899 |
| 26 | 232900 | 233067 | 25 | 233068 | 235354 |
| 27 | 235355 | 235459 | | | |
| | Position in | | | Position in | |
| | SEQ ID No 3 | | | SEQ ID No 3 | |
| Exon | Beginning | End | Intron | Beginning | End |
| 28 | 3895 | 4001 | 26 | 4002 | 9610 |
| 29 | 9611 | 9731 | 27 | 9732 | 9815 |
| 30 | 9816 | 9914 | 28 | 9915 | 15775 |
| 31 | 15776 | 15869 | 29 | 15870 | 16381 |
| 32 | 16382 | 16488 | 30 | 16489 | 16696 |
| 33 | 16697 | 16771 | 31 | 16772 | 17933 |
| 34 | 17934 | 18053 | 32 | 18054 | 23643 |
| 35 | 23644 | 23712 | 33 | 23713 | 24927 |
| 36 | 24928 | 25076 | 34 | 25077 | 25912 |
| 37 | 25913 | 26006 | 35 | 26007 | 30766 |
| 38 | 30767 | 30899 | 36 | 30900 | 31560 |
| 39 | 31561 | 31676 | 37 | 31677 | 34043 |
| 40 | 34044 | 34201 | 38 | 34202 | 37492 |
| 41 | 37493 | 37643 | 39 | 37644 | 39651 |
| 42 | 39652 | 39801 | 40 | 39802 | 41562 |
| 43 | 41563 | 41680 | 41 | 41681 | 44130 |
| 44 | 44131 | 45841 | | | |

Thus, the invention embodies purified, isolated, or recombinant polynucleotides comprising a nucleotide sequence selected from the group consisting of each of the 44 exons of the CanIon gene and each of the sequences complementary thereto. The invention also provides purified, isolated, or recombinant nucleic acids comprising a combination of at least two exons of the CanIon gene, wherein the polynucleotides are arranged within the nucleic acid, from the 5′-end to the 3′-end of said nucleic acid, in the same order as in SEQ ID No 1 to 3.

Intron 1 refers to the nucleotide sequence located between Exon 1 and Exon 2, and so on. The position of the introns is detailed in Table A. Thus, the invention embodies purified, isolated, or recombinant polynucleotides comprising a nucleotide sequence selected from the group consisting of the 43 introns of the CanIon gene, or a sequence complementary thereto.

While this section is entitled “Genomic Sequences of CanIon,” it should be noted that nucleic acid fragments of any size and sequence may also be comprised by the polynucleotides described in this section, flanking the genomic sequences of CanIon on either side or between two or more such genomic sequences.

CanIon cDNA Sequences

The expression of the CanIon gene has been shown to lead to the production of at least one mRNA species, the nucleic acid sequence of which is set forth in SEQ ID No 4.

Another object of the invention is a purified, isolated, or recombinant nucleic acid comprising the nucleotide sequence of SEQ ID No 4, complementary sequences thereto, as well as allelic variants, and fragments thereof. Moreover, preferred polynucleotides of the invention include purified, isolated, or recombinant CanIon cDNAs consisting of, consisting essentially of, or comprising the sequence of SEQ ID No 4. Particularly preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, 1000, 2000, 3000, 4000, 5000 or 6000 nucleotides of SEQ ID No 4 or the complements thereof. In preferred embodiments, said contiguous span comprises a CanIon-related biallelic marker; preferably selected from the group consisting of A12 and A16.

The invention also pertains to a purified or isolated nucleic acid comprising a polynucleotide having at least 95% nucleotide identity with a polynucleotide of SEQ ID No 4, advantageously 99% nucleotide identity, preferably 99.5% nucleotide identity and most preferably 99.8% nucleotide identity with a polynucleotide of SEQ ID No 4, or a sequence complementary thereto or a biologically active fragment thereof.

Another object of the invention relates to purified, isolated or recombinant nucleic acids comprising a polynucleotide that hybridizes, under the stringent hybridization conditions defined herein, with a polynucleotide of SEQ ID No 4, or a sequence complementary thereto or a variant thereof or a biologically active fragment thereof.

A further object of the invention relates to an isolated, purified, or recombinant polynucleotide which encodes a CanIon polypeptide comprising a contiguous span of at least 6 amino acids of SEQ ID No 5, wherein said contiguous span includes at least 1, 2, 3, 5 or 10 of the amino acid positions 277, 338, 574, 678, 680, 683, 691, 692, 695, 696, 697, 894, 1480, 1481, 1483, 1484, 1485, 1630, 1631, 1632, 1636, 1660, 1667, 1707, 1709 of SEQ ID No 5. Also encompassed is an isolated, purified, or recombinant polynucleotide which encodes a CanIon polypeptide comprising the amino acid sequence of SEQ ID No 5, or derivatives or biologically active fragments thereof, as well as an isolated, purified, or recombinant polynucleotide which encodes a CanIon polypeptide at least 80, 85, 90, 95, 98, 99, 99.5 or 99.8% identical to the amino acid sequence of SEQ ID No 5.

The cDNA of SEQ ID No 4 includes a 5′-UTR region starting from the nucleotide at position 1 and ending at the nucleotide in position 65 of SEQ ID No 4. The cDNA of SEQ ID No cDNA includes a 3′-UTR region starting from the nucleotide at position 5283 and ending at the nucleotide at position 6799 of SEQ ID No 4.

Consequently, the invention concerns a purified, isolated, and recombinant nucleic acid comprising a nucleotide sequence of the 5′UTR of the CanIon cDNA, a sequence complementary thereto, or an allelic variant thereof. The invention also concerns a purified, isolated, and recombinant nucleic acid comprising a nucleotide sequence of the 3′UTR of the CanIon cDNA, a sequence complementary thereto, or an allelic variant thereof.

While this section is entitled “CanIon cDNA Sequences,” it should be noted that nucleic acid fragments of any size and sequence may also be comprised by the polynucleotides described in this section, flanking the genomic sequences of CanIon on either side or between two or more such genomic sequences.

### Coding Regions

The CanIon open reading frame is contained in the corresponding mRNA of SEQ ID No cDNA. More precisely, the effective CanIon coding sequence (CDS) includes the region between nucleotide position 66 (first nucleotide of the ATG codon) and nucleotide position 5282 (end nucleotide of the TGA codon) of SEQ ID No 4. The present invention also embodies isolated, purified, and recombinant polynucleotides which encode a polypeptides comprising a contiguous span of at least 6 amino acids, preferably at least 8 or 10 amino acids, more preferably at least 12, 15, 20, 25, 30, 40, 50, 100, 200, 300, 400, 500, 700 or 1000 amino acids of SEQ ID No 5.

The above disclosed polynucleotide that contains the coding sequence of the CanIon gene may be expressed in a desired host cell or a desired host organism, when this polynucleotide is placed under the control of suitable expression signals. The expression signals may be either the expression signals contained in the regulatory regions in the CanIon gene of the invention or in contrast the signals may be exogenous regulatory nucleic sequences. Such a polynucleotide, when placed under the suitable expression signals, may also be inserted in a vector for its expression and/or amplification.

### Regulatory Sequences of CanIon

As mentioned, the genomic sequence of the CanIon gene contains regulatory sequences both in the non-coding 5′-flanking region and in the non-coding 3-′-flanking region that border the CanIon coding region containing the 44 exons of this gene.

Polynucleotides derived from the 5′ and 3′ regulatory regions are useful in order to detect the presence of at least a copy of a CanIon nucleotide sequence or a fragment thereof in a test sample.

The promoter activity of the 5′ regulatory regions contained in CanIon can be assessed as described as follows. In order to identify the relevant biologically active polynucleotide fragments or variants of SEQ ID No 1, one of skill in the art may refer to Sambrook et al. (1989) which describes the use of a recombinant vector carrying a marker gene (i.e. beta galactosidase, chloramphenicol acetyl transferase, etc.), the expression of which can be detected when placed under the control of a biologically active polynucleotide fragments or variants of SEQ ID No 1. Genomic sequences located upstream of the first exon of the CanIon gene are cloned into a suitable promoter reporter vector, such as the pSEAP-Basic, pSEAP-Enhancer, pβgal-Basic, pβgal-Enhancer, or pEGFP-1 Promoter Reporter vectors available from Clontech, or pGL2-basic or pGL3-basic promoterless luciferase reporter gene vector from Promega. Briefly, each of these promoter reporter vectors include multiple cloning sites positioned upstream of a reporter gene encoding a readily assayable protein such as secreted alkaline phosphatase, luciferase, β galactosidase, or green fluorescent protein. The sequences upstream the CanIon coding region are inserted into the cloning sites upstream of the reporter gene in both orientations and introduced into an appropriate host cell. The level of reporter protein is assayed and compared to the level obtained from a vector which lacks an insert in the cloning site. The presence of an elevated expression level in the vector containing the insert in comparison to the level in the control vector indicates the presence of a promoter in the insert. If necessary, the upstream sequences can be cloned into vectors which contain an enhancer for increasing transcription levels from weak promoter sequences. A significant level of expression above that observed with the vector lacking an insert indicates that a promoter sequence is present in the inserted upstream sequence.

Promoter sequence within the upstream genomic DNA may be further defined by constructing nested 5′ and/or 3′ deletions in the upstream DNA using conventional techniques such as Exonuclease III or appropriate restriction endonuclease digestion. The resulting deletion fragments can be inserted into the promoter reporter vector to determine whether the deletion has reduced or obliterated promoter activity, such as described, for example, by Coles et al. (1998), the disclosure of which is incorporated herein by reference in its entirety. In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. This type of assay is well-known to those skilled in the art and is described in WO 97/17359, U.S. Pat. No. 5,374,544; EP 582 796; U.S. Pat. No. 5,698,389; U.S. Pat. No. 5,643,746; U.S. Pat. No. 5,502,176; and U.S. Pat. No. 5,266,488; the disclosures of which are incorporated by reference herein in their entirety.

The strength and the specificity of the promoter of the CanIon gene can be assessed through the expression levels of a detectable polynucleotide operably linked to the CanIon promoter in different types of cells and tissues. The detectable polynucleotide may be either a polynucleotide that specifically hybridizes with a predefined oligonucleotide probe, or a polynucleotide encoding a detectable protein, including a CanIon polypeptide or a fragment or a variant thereof. This type of assay is well-known to those skilled in the art and is described in U.S. Pat. No. 5,502,176; and U.S. Pat. No. 5,266,488; the disclosures of which are incorporated by reference herein in their entirety. Some of the methods are discussed in more detail below.

Polynucleotides carrying the regulatory elements located at the 5′ end and at the 3′ end of the CanIon coding region may be advantageously used to control the transcriptional and translational activity of an heterologous polynucleotide of interest.

Thus, the present invention also concerns a purified or isolated nucleic acid comprising a polynucleotide which is selected from the group consisting of the 5′ and 3′ regulatory regions, or a sequence complementary thereto or a biologically active fragment or variant thereof. In preferred embodiments, “5′ regulatory region” is located in the nucleotide sequence located between positions 1 and 2000 of SEQ ID No 1. The “3′ regulatory region” is located in the nucleotide sequence located between positions 45842 and 47841 of SEQ ID No 3.

The invention also pertains to a purified or isolated nucleic acid comprising a polynucleotide having at least 95% nucleotide identity with a polynucleotide selected from the group consisting of the 5′ and 3′ regulatory regions, advantageously 99% nucleotide identity, preferably 99.5% nucleotide identity and most preferably 99.8% nucleotide identity with a polynucleotide selected from the group consisting of the 5′ and 3′ regulatory regions, or a sequence complementary thereto or a variant thereof or a biologically active fragment thereof.

Another object of the invention consists of purified, isolated or recombinant nucleic acids comprising a polynucleotide that hybridizes, under the stringent hybridization conditions defined herein, with a polynucleotide selected from the group consisting of the nucleotide sequences of the 5′- and 3′ regulatory regions, or a sequence complementary thereto or a variant thereof or a biologically active fragment thereof.

Preferred fragments of the 5′ regulatory region have a length of about 1500 or 1000 nucleotides, preferably of about 500 nucleotides, more preferably about 400 nucleotides, even more preferably 300 nucleotides and most preferably about 200 nucleotides.

Preferred fragments of the 3′ regulatory region are at least 50, 100, 150, 200, 300 or 400 bases in length.

“Biologically active” polynucleotide derivatives of SEQ ID Nos 1 and 3 are polynucleotides comprising or alternatively consisting in a fragment of said polynucleotide which is functional as a regulatory region for expressing a recombinant polypeptide or a recombinant polynucleotide in a recombinant cell host. It could act either as an enhancer or as a repressor.

For the purpose of the invention, a nucleic acid or polynucleotide is “functional” as a regulatory region for expressing a recombinant polypeptide or a recombinant polynucleotide if said regulatory polynucleotide contains nucleotide sequences which contain transcriptional and translational regulatory information, and such sequences are “operably linked” to nucleotide sequences which encode the desired polypeptide or the desired polynucleotide.

The regulatory polynucleotides of the invention may be prepared from the nucleotide sequence of SEQ ID Nos 1 and 3 by cleavage using suitable restriction enzymes, as described for example in the book of Sambrook et al. (1989). The regulatory polynucleotides may also be prepared by digestion of SEQ ID Nos 1 and 3 by an exonuclease enzyme, such as Bal31 (Wabiko et al., 1986). These regulatory polynucleotides can also be prepared by nucleic acid chemical synthesis, as described elsewhere in the specification.

The regulatory polynucleotides according to the invention may be part of a recombinant expression vector that may be used to express a coding sequence in a desired host cell or host organism. The recombinant expression vectors according to the invention are described elsewhere in the specification.

A preferred 5′-regulatory polynucleotide of the invention includes the 5′-untranslated region (5′-UTR) of the CanIon cDNA, or a biologically active fragment or variant thereof.

A preferred 3′-regulatory polynucleotide of the invention includes the 3′-untranslated region (3′-UTR) of the CanIon cDNA, or a biologically active fragment or variant thereof.

A further object of the invention consists of a purified or isolated nucleic acid comprising:
a) a nucleic acid comprising a regulatory nucleotide sequence selected from the group consisting of:
(i) a nucleotide sequence comprising a polynucleotide of the 5′ regulatory region or a complementary sequence thereto;
(ii) a nucleotide sequence comprising a polynucleotide having at least 95% of nucleotide identity with the nucleotide sequence of the 5′ regulatory region or a complementary sequence thereto;
(iii) a nucleotide sequence comprising a polynucleotide that hybridizes under stringent hybridization conditions with the nucleotide sequence of the 5′ regulatory region or a complementary sequence thereto; and
(iv) a biologically active fragment or variant of the polynucleotides in (i), (ii) and (iii);
b) a polynucleotide encoding a desired polypeptide or a nucleic acid of interest, operably linked to the nucleic acid defined in (a) above;
c) Optionally, a nucleic acid comprising a 3′-regulatory polynucleotide, preferably a 3′-regulatory polynucleotide of the CanIon gene.

In a specific embodiment of the nucleic acid defined above, said nucleic acid includes the 5′-untranslated region (5′-UTR) of the CanIon cDNA, or a biologically active fragment or variant thereof.

In a second specific embodiment of the nucleic acid defined above, said nucleic acid includes the 3′-untranslated region (3′-UTR) of the CanIon cDNA, or a biologically active fragment or variant thereof.

The regulatory polynucleotide of the 5′ regulatory region, or its biologically active fragments or variants, is operably linked at the 5′-end of the polynucleotide encoding the desired polypeptide or polynucleotide.

The regulatory polynucleotide of the 3′ regulatory region, or its biologically active fragments or variants, is advantageously operably linked at the 3′-end of the polynucleotide encoding the desired polypeptide or polynucleotide.

The desired polypeptide encoded by the above-described nucleic acid may be of various nature or origin, encompassing proteins of prokaryotic or eukaryotic origin. Among the polypeptides expressed under the control of a CanIon regulatory region include bacterial, fungal or viral antigens. Also encompassed are eukaryotic proteins such as intracellular proteins, like “house keeping” proteins, membrane-bound proteins, like receptors, and secreted proteins like endogenous mediators such as cytokines. The desired polypeptide may be the CanIon protein, especially the protein of the amino acid sequence of SEQ ID No 5, or a fragment or a variant thereof.

The desired nucleic acids encoded by the above-described polynucleotide, usually an RNA molecule, may be complementary to a desired coding polynucleotide, for example to the CanIon coding sequence, and thus useful as an antisense polynucleotide.

Such a polynucleotide may be included in a recombinant expression vector in order to express the desired polypeptide or the desired nucleic acid in host cell or in a host organism. Suitable recombinant vectors that contain a polynucleotide such as described herein are disclosed elsewhere in the specification.

### Polynucleotide Constructs

The terms “polynucleotide construct” and “recombinant polynucleotide” are used interchangeably herein to refer to linear or circular, purified or isolated polynucleotides that have been artificially designed and which comprise at least two nucleotide sequences that are not found as contiguous nucleotide sequences in their initial natural environment.

DNA Construct that Enables Directing Temporal and Spatial CanIon Gene Expression in Recombinant Cell Hosts and in Transgenic Animals.

In order to study the physiological and phenotypic consequences of a lack of synthesis of the CanIon protein, both at the cellular level and at the multi cellular organism level, the invention also encompasses DNA constructs and recombinant vectors enabling a conditional expression of a specific allele of the CanIon genomic sequence or cDNA and also of a copy of this genomic sequence or cDNA harboring substitutions, deletions, or additions of one or more bases as regards to the CanIon nucleotide sequence of SEQ ID Nos 1 to 4, or a fragment thereof, these base substitutions, deletions or additions being located either in an exon, an intron or a regulatory sequence, but preferably in the 5′-regulatory sequence or in an exon of the CanIon genomic sequence or within the CanIon cDNA of SEQ ID No 4. In a preferred embodiment, the CanIon sequence comprises a biallelic marker of the present invention. In a preferred embodiment, the CanIon sequence comprises a biallelic marker of the present invention, preferably one of the biallelic markers A1 to A17.

The present invention embodies recombinant vectors comprising any one of the polynucleotides described in the present invention. More particularly, the polynucleotide constructs according to the present invention can comprise any of the polynucleotides described in the “Genomic Sequences Of The CanIon Gene” section, the “CanIon cDNA Sequences” section, the “Coding Regions” section, and the “Oligonucleotide Probes And Primers” section.

A first preferred DNA construct is based on the tetracycline resistance operon tet from E. coli transposon Tn10 for controlling the CanIon gene expression, such as described by Gossen et al. (1992, 1995) and Furth et al. (1994). Such a DNA construct contains seven tel operator sequences from Tn 10 (tetop) that are fused to either a minimal promoter or a 5′-regulatory sequence of the CanIon gene, said minimal promoter or said CanIon regulatory sequence being operably linked to a polynucleotide of interest that codes either for a sense or an antisense oligonucleotide or for a polypeptide, including a CanIon polypeptide or a peptide fragment thereof. This DNA construct is functional as a conditional expression system for the nucleotide sequence of interest when the same cell also comprises a nucleotide sequence coding for either the wild type (tTA) or the mutant (rTA) repressor fused to the activating domain of viral protein VP 16 of herpes simplex virus, placed under the control of a promoter, such as the HCMVIE1 enhancer/promoter or the MMTV-LTR. Indeed, a preferred DNA construct of the invention comprise both the polynucleotide containing the tet operator sequences and the polynucleotide containing a sequence coding for the tTA or the rTA repressor.

In a specific embodiment, the conditional expression DNA construct contains the sequence encoding the mutant tetracycline repressor rTA, the expression of the polynucleotide of interest is silent in the absence of tetracycline and induced in its presence.

DNA Constructs Allowing Homologous Recombination: Replacement Vectors

A second preferred DNA construct will comprise, from 5′-end to 3′-end: (a) a first nucleotide sequence that is comprised in the CanIon genomic sequence; (b) a nucleotide sequence comprising a positive selection marker, such as the marker for neomycine resistance (neo); and (c) a second nucleotide sequence that is comprised in the CanIon genomic sequence, and is located on the genome downstream the first CanIon nucleotide sequence (a).

In a preferred embodiment, this DNA construct also comprises a negative selection marker located upstream the nucleotide sequence (a) or downstream the nucleotide sequence (c). Preferably, the negative selection marker comprises the thymidine kinase (tk) gene (Thomas et al., 1986), the hygromycine beta gene (Te Riele et al., 1990), the hprt gene (Van der Lugt et al., 1991; Reid et al., 1990) or the Diphteria toxin A fragment (Dt-A) gene (Nada et al., 1993; Yagi et al. 1990). Preferably, the positive selection marker is located within a CanIon exon sequence so as to interrupt the sequence encoding a CanIon protein. These replacement vectors are described, for example, by Thomas et al. (1986; 1987), Mansour et al. (1988) and Koller et al. (1992).

The first and second nucleotide sequences (a) and (c) may be indifferently located within a CanIon regulatory sequence, an intronic sequence, an exon sequence or a sequence containing both regulatory and/or intronic and/or exon sequences. The size of the nucleotide sequences (a) and (c) ranges from 1 to 50 kb, preferably from 1 to 10 kb, more preferably from 2 to 6 kb and most preferably from 2 to 4 kb.

DNA Constructs Allowing Homologous Recombination: Cre-LoxP System.

These new DNA constructs make use of the site specific recombination system of the P1 phage. The P1 phage possesses a recombinase called Cre which interacts specifically with a 34 base pairs loxP site. The loxP site is composed of two palindromic sequences of 13 bp separated by a 8 bp conserved sequence (Hoess et al., 1986). The recombination by the Cre enzyme between two loxP sites having an identical orientation leads to the deletion of the DNA fragment.

The Cre-loxP system used in combination with a homologous recombination technique has been first described by Gu et al. (1993, 1994). Briefly, a nucleotide sequence of interest to be inserted in a targeted location of the genome harbors at least two loxP sites in the same orientation and located at the respective ends of a nucleotide sequence to be excised from the recombinant genome. The excision event requires the presence of the recombinase (Cre) enzyme within the nucleus of the recombinant cell host. The recombinase enzyme may be brought at the desired time either by (a) incubating the recombinant cell hosts in a culture medium containing this enzyme, by injecting the Cre enzyme directly into the desired cell, such as described by Araki et al. (1995), or by lipofection of the enzyme into the cells, such as described by Baubonis et al. (1993); (b) transfecting the cell host with a vector comprising the Cre coding sequence operably linked to a promoter functional in the recombinant cell host, which promoter being optionally inducible, said vector being introduced in the recombinant cell host, such as described by Gu et al. (1993) and Sauer et al. (1988); (c) introducing in the genome of the cell host a polynucleotide comprising the Cre coding sequence operably linked to a promoter functional in the recombinant cell host, which promoter is optionally inducible, and said polynucleotide being inserted in the genome of the cell host either by a random insertion event or an homologous recombination event, such as described by Gu et al. (1994).

In a specific embodiment, the vector containing the sequence to be inserted in the CanIon gene by homologous recombination is constructed in such a way that selectable markers are flanked by loxP sites of the same orientation, it is possible, by treatment by the Cre enzyme, to eliminate the selectable markers while leaving the CanIon sequences of interest that have been inserted by an homologous recombination event. Again, two selectable markers are needed: a positive selection marker to select for the recombination event and a negative selection marker to select for the homologous recombination event. Vectors and methods using the Cre-loxP system are described by Zou et al. (1994).

Thus, a third preferred DNA construct of the invention comprises, from 5′-end to 3′-end: (a) a first nucleotide sequence that is comprised in the CanIon genomic sequence; (b) a nucleotide sequence comprising a polynucleotide encoding a positive selection marker, said nucleotide sequence comprising additionally two sequences defining a site recognized by a recombinase, such as a loxP site, the two sites being placed in the same orientation; and (c) a second nucleotide sequence that is comprised in the CanIon genomic sequence, and is located on the genome downstream of the first CanIon nucleotide sequence (a).

The sequences defining a site recognized by a recombinase, such as a loxP site, are preferably located within the nucleotide sequence (b) at suitable locations bordering the nucleotide sequence for which the conditional excision is sought. In one specific embodiment, two loxP sites are located at each side of the positive selection marker sequence, in order to allow its excision at a desired time after the occurrence of the homologous recombination event.

In a preferred embodiment of a method using the third DNA construct described above, the excision of the polynucleotide fragment bordered by the two sites recognized by a recombinase, preferably two loxP sites, is performed at a desired time, due to the presence within the genome of the recombinant host cell of a sequence encoding the Cre enzyme operably linked to a promoter sequence, preferably an inducible promoter, more preferably a tissue-specific promoter sequence and most preferably a promoter sequence which is both inducible and tissue-specific, such as described by Gu et al. (1994).

The presence of the Cre enzyme within the genome of the recombinant cell host may result from the breeding of two transgenic animals, the first transgenic animal bearing the CanIon-derived sequence of interest containing the loxP sites as described above and the second transgenic animal bearing the Cre coding sequence operably linked to a suitable promoter sequence, such as described by Gu et al. (1994).

Spatio-temporal control of the Cre enzyme expression may also be achieved with an adenovirus based vector that contains the Cre gene thus allowing infection of cells, or in vivo infection of organs, for delivery of the Cre enzyme, such as described by Anton and Graham (1995) and Kanegae et al. (1995).

The DNA constructs described above may be used to introduce a desired nucleotide sequence of the invention, preferably a CanIon genomic sequence or a CanIon cDNA sequence, and most preferably an altered copy of a CanIon genomic or cDNA sequence, within a predetermined location of the targeted genome, leading either to the generation of an altered copy of a targeted gene (knock-out homologous recombination) or to the replacement of a copy of the targeted gene by another copy sufficiently homologous to allow an homologous recombination event to occur (knock-in homologous recombination). In a specific embodiment, the DNA constructs described above may be used to introduce a CanIon genomic sequence or a CanIon cDNA sequence comprising at least one biallelic marker of the present invention, preferably at least one biallelic marker selected from the group consisting of A1 to A17.

Nuclear Antisense DNA Constructs

Other compositions containing a vector of the invention comprising an oligonucleotide fragment of the nucleic sequence SEQ ID No 4, preferably a fragment including the start codon of the CanIon gene, as an antisense tool that inhibits the expression of the corresponding CanIon gene. Preferred methods using antisense polynucleotide according to the present invention are the procedures described by Sczakiel et al. (1995) or those described in PCT Application No WO 95/24223, the disclosures of which are incorporated by reference herein in their entirety.

Preferably, the antisense tools are chosen among the polynucleotides (15-200 bp long) that are complementary to the 5′ end of the CanIon mRNA. In one embodiment, a combination of different antisense polynucleotides complementary to different parts of the desired targeted gene are used.

Preferred antisense polynucleotides according to the present invention are complementary to a sequence of the mRNAs of CanIon that contains either the translation initiation codon ATG or a splicing site. Further preferred antisense polynucleotides according to the invention are complementary of the splicing site of the CanIon mRNA.

Preferably, the antisense polynucleotides of the invention have a 3′ polyadenylation signal that has been replaced with a self-cleaving ribozyme sequence, such that RNA polymerase II transcripts are produced without poly(A) at their 3′ ends, these antisense polynucleotides being incapable of export from the nucleus, such as described by Liu et al. (1994). In a preferred embodiment, these CanIon antisense polynucleotides also comprise, within the ribozyme cassette, a histone stem-loop structure to stabilize cleaved transcripts against 3′-5′ exonucleolytic degradation, such as the structure described by Eckner et al. (1991).

### Oligonucleotide Probes and Primers

Polynucleotides derived from the CanIon gene are useful in order to detect the presence of at least a copy of a nucleotide sequence of SEQ ID Nos 1 to 4 and 6, or a fragment, complement, or variant thereof in a test sample.

Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, 1000, 2000, 5000, 10000 or 20000 nucleotides of SEQ ID Nos 1 to 3 or the complements thereof. Further preferred probes and primers of the invention include isolated, purified, or recombinant polynucleotides, wherein said contiguous span comprises a biallelic marker selected from the group consisting of A1 to A17.

Another object of the invention is a purified, isolated, or recombinant nucleic acid comprising the nucleotide sequence of SEQ ID No 4, complementary sequences thereto, as well as allelic variants, and fragments thereof. Moreover, preferred probes and primers of the invention include purified, isolated, or recombinant CanIon cDNA consisting of, consisting essentially of, or comprising the sequence of SEQ ID No 4. Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, 1000, 2000, 3000, 4000, 5000 or 6000 nucleotides of SEQ ID No 4 or the complements thereof. Further preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, 1000, 2000, 3000, 4000, 5000 or 6000 nucleotides of SEQ ID No 4 or the complements thereof, wherein said contiguous span comprises a biallelic marker selected from the group consisting of A12 and A16.

In further embodiments, probes and primers of the invention include isolated, purified, or recombinant polynucleotides comprising a contiguous span of at least 12, 15, 18, 20, 25, 30, 35, 40, 50, 60, 70, 80, 90, 100, 150, 200, 300 or 400 nucleotides of SEQ ID No 6 or the complements thereof. In preferred embodiments, said contiguous span of SEQ ID No 6 comprises a biallelic marker A18.

Thus, the invention also relates to nucleic acid probes characterized in that they hybridize specifically, under the stringent hybridization conditions defined above, with a nucleic acid selected from the group consisting of the human CanIon nucleotide sequences of SEQ ID Nos 1 to 3, or a variant thereof or a sequence complementary thereto.

In one embodiment the invention encompasses isolated, purified, and recombinant polynucleotides consisting of, or consisting essentially of a contiguous span of 8 to 50 nucleotides of any one of SEQ ID Nos 1 to 4 and 6, and the complement thereof, wherein said span includes a CanIon-related biallelic marker in said sequence; optionally, wherein said CanIon-related biallelic marker is selected from the group consisting of A1 to A18, and the complements thereof, or optionally the biallelic markers in linkage disequilibrium therewith. Optionally, wherein said contiguous span is 18 to 35 nucleotides in length and said biallelic marker is within 4 nucleotides of the center of said polynucleotide; optionally, wherein said polynucleotide consists of said contiguous span and said contiguous span is 25 nucleotides in length and said biallelic marker is at the center of said polynucleotide; optionally, wherein the 3′ end of said contiguous span is present at the 3′ end of said polynucleotide; and optionally, wherein the 3′ end of said contiguous span is located at the 3′ end of said polynucleotide and said biallelic marker is present at the 3′ end of said polynucleotide. In a preferred embodiment, said probes comprises, consists of, or consists essentially of a sequence selected from the following sequences: P1 to P18 and the complementary sequences thereto.

In another embodiment the invention encompasses isolated, purified and recombinant polynucleotides comprising, consisting of, or consisting essentially of a contiguous span of 8 to 50 nucleotides of SEQ ID Nos 1 to 4, or the complements thereof, wherein the 3′ end of said contiguous span is located at the 3′ end of said polynucleotide, and wherein the 3′ end of said polynucleotide is located within 20 nucleotides upstream of a CanIon-related biallelic marker in said sequence; optionally, wherein said CanIon-related biallelic marker is selected from the group consisting of A1 to A18, and the complements thereof, or optionally the biallelic markers in linkage disequilibrium therewith; optionally, wherein the 3′ end of said polynucleotide is located 1 nucleotide upstream of said CanIon-related biallelic marker in said sequence; and optionally, wherein said polynucleotide consists essentially of a sequence selected from the following sequences: D1 to D18 and E1 to E18.

In a further embodiment, the invention encompasses isolated, purified, or recombinant polynucleotides comprising, consisting of, or consisting essentially of a sequence selected from the following sequences: B1 to B17 and C1 to C17.

In an additional embodiment, the invention encompasses polynucleotides for use in hybridization assays, sequencing assays, and enzyme-based mismatch detection assays for determining the identity of the nucleotide at a CanIon-related biallelic marker in SEQ ID Nos 1 to 4 and 6, or the complements thereof, as well as polynucleotides for use in amplifying segments of nucleotides comprising a CanIon-related biallelic marker in SEQ ID Nos 1 to 4 and 6, or the complements thereof; optionally, wherein said CanIon-related biallelic marker is selected from the group consisting of A1 to A18, and the complements thereof, or optionally the biallelic markers in linkage disequilibrium therewith.

The invention concerns the use of the polynucleotides according to the invention for determining the identity of the nucleotide at a CanIon-related biallelic marker, preferably in hybridization assay, sequencing assay, microsequencing assay, or an enzyme-based mismatch detection assay and in amplifying segments of nucleotides comprising a CanIon-related biallelic marker.

A probe or a primer according to the invention has between 8 and 1000 nucleotides in length, or is specified to be at least 12, 15, 18, 20, 25, 35, 40, 50, 60, 70, 80, 100, 250, 500 or 1000 nucleotides in length. More particularly, the length of these probes and primers can range from 8, 10, 15, 20, or 30 to 100 nucleotides, preferably from 10 to 50, more preferably from 15 to 30 nucleotides. Shorter probes and primers tend to lack specificity for a target nucleic acid sequence and generally require cooler temperatures to form sufficiently stable hybrid complexes with the template. Longer probes and primers are expensive to produce and can sometimes self-hybridize to form hairpin structures. The appropriate length for primers and probes under a particular set of assay conditions may be empirically determined by one of skill in the art. A preferred probe or primer consists of a nucleic acid comprising a polynucleotide selected from the group of the nucleotide sequences of P1 to P18 and the complementary sequence thereto, B1 to B17, C1 to C17, D1 to D18, E1 to E18, for which the respective locations in the sequence listing are provided in Tables 1, 2, and 3.

The formation of stable hybrids depends on the melting temperature (Tm) of the DNA. The Tm depends on the length of the primer or probe, the ionic strength of the solution and the G+C content. The higher the G+C content of the primer or probe, the higher is the melting temperature because G:C pairs are held by three H bonds whereas A:T pairs have only two. The GC content in the probes of the invention usually ranges between 10 and 75%, preferably between 35 and 60%, and more preferably between 40 and 55%.

The primers and probes can be prepared by any suitable method, including, for example, cloning and restriction of appropriate sequences and direct chemical synthesis by a method such as the phosphodiester method of Narang et al. (1979), the phosphodiester method of Brown et al. (1979), the diethylphosphoramidite method of Beaucage et al. (1981) and the solid support method described in EP 0 707 592.

Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified, U.S. patent application Ser. No. 07/049,061 filed Apr. 19, 1993 describes modifications, which can be used to render a probe non-extendable.

Any of the polynucleotides of the present invention can be labeled, if desired, by incorporating any label known in the art to be detectable by spectroscopic, photochemical, biochemical, immunochemical, or chemical means. For example, useful labels include radioactive substances (including, ³²P, ³⁵S, ³H, ¹²⁵I), fluorescent dyes (including, 5-bromodesoxyuridin, fluorescein, acetylaminofluorene, digoxigenin) or biotin. Preferably, polynucleotides are labeled at their 3′ and 5′ ends. Examples of non-radioactive labeling of nucleic acid fragments are described in the French patent No. FR-7810975 or by Urdea et al. (1988) or Sanchez-Pescador et al. (1988). In addition, the probes according to the present invention may have structural characteristics such that they allow the signal amplification, such structural characteristics being, for example, branched DNA probes as those described by Urdea et al. (1991) or in the European patent No. EP 0 225 807 (Chiron).

A label can also be used to capture the primer, so as to facilitate the immobilization of either the primer or a primer extension product, such as amplified DNA, on a solid support. A capture label is attached to the primers or probes and can be a specific binding member which forms a binding pair with the solid's phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

The probes of the present invention are useful for a number of purposes. They can be notably used in Southern hybridization to genomic DNA. The probes can also be used to detect PCR amplification products. They may also be used to detect mismatches in the CanIon gene or mRNA using other techniques. They can also be used to detect expression of a CanIon gene, e.g. in a Northern blot.

Any of the polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, Duracytes® and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Consequently, the invention also comprises a method for detecting the presence of a nucleic acid comprising a nucleotide sequence selected from a group consisting of SEQ ID Nos 1 to 4 and 6, a fragment or a variant thereof and a complementary sequence thereto in a sample, said method comprising the following steps of:

a) bringing into contact a nucleic acid probe or a plurality of nucleic acid probes which can hybridize with a nucleotide sequence included in a nucleic acid selected form the group consisting of the nucleotide sequences of SEQ ID Nos 1 to 4 and 6, a fragment or a variant thereof and a complementary sequence thereto and the sample to be assayed; and

b) detecting the hybrid complex formed between the probe and a nucleic acid in the sample.

The invention further concerns a kit for detecting the presence of a nucleic acid comprising a nucleotide sequence selected from a group consisting of SEQ ID Nos 1 to 4 and 6, a fragment or a variant thereof and a complementary sequence thereto in a sample, said kit comprising:

a) a nucleic acid probe or a plurality of nucleic acid probes which can hybridize with a nucleotide sequence included in a nucleic acid selected form the group consisting of the nucleotide sequences of SEQ ID Nos 1 to 4 and 6, a fragment or a variant thereof and a complementary sequence thereto; and

b) optionally, the reagents necessary for performing the hybridization reaction.

In a first preferred embodiment of this detection method and kit, said nucleic acid probe or the plurality of nucleic acid probes are labeled with a detectable molecule. In a second preferred embodiment of said method and kit, said nucleic acid probe or the plurality of nucleic acid probes has been immobilized on a substrate. In a third preferred embodiment, the nucleic acid probe or the plurality of nucleic acid probes comprise either a sequence which is selected from the group consisting of the nucleotide sequences of P1 to P18 and the complementary sequence thereto, B1 to B17, C1 to C17, D1 to D18, E1 to E18 or a biallelic marker selected from the group consisting of A1 to A18 and the complements thereto.

Oligonucleotide Arrays

A substrate comprising a plurality of oligonucleotide primers or probes of the invention may be used either for detecting or amplifying targeted sequences in the CanIon gene and may also be used for detecting mutations in the coding or in the non-coding sequences of the CanIon gene.

Any polynucleotide provided herein may be attached in overlapping areas or at random locations on the solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotide makes these “addressable” arrays particularly useful in hybridization assays. Any addressable array technology known in the art can be employed with the polynucleotides of the invention. One particular embodiment of these polynucleotide arrays is known as the Genechips™, and has been generally described in U.S. Pat. No. 5,143,854; PCT publications WO 90/15070 and 92/10092. These arrays may generally be produced using mechanical synthesis methods or light directed synthesis methods which incorporate a combination of photolithographic methods and solid phase oligonucleotide synthesis (Fodor et al., 1991). The immobilization of arrays of oligonucleotides on solid supports has been rendered possible by the development of a technology generally identified as “Very Large Scale Immobilized Polymer Synthesis” (VLSIPS™) in which, typically, probes are immobilized in a high density array on a solid surface of a chip. Examples of VLSIPS™ technologies are provided in U.S. Pat. Nos. 5,143,854; and 5,412,087 and in PCT Publications WO 90/15070, WO 92/10092 and WO 95/11995, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256, the disclosures of which are incorporated herein by reference in their entireties.

In another embodiment of the oligonucleotide arrays of the invention, an oligonucleotide probe matrix may advantageously be used to detect mutations occurring in the CanIon gene and preferably in its regulatory region. For this particular purpose, probes are specifically designed to have a nucleotide sequence allowing their hybridization to the genes that carry known mutations (either by deletion, insertion or substitution of one or several nucleotides). By known mutations, it is meant, mutations on the CanIon gene that have been identified according, for example to the technique used by Huang et al. (1996) or Samson et al. (1996).

Another technique that is used to detect mutations in the CanIon gene is the use of a high-density DNA array. Each oligonucleotide probe constituting a unit element of the high density DNA array is designed to match a specific subsequence of the CanIon genomic DNA or cDNA. Thus, an array consisting of oligonucleotides complementary to subsequences of the target gene sequence is used to determine the identity of the target sequence with the wild gene sequence, measure its amount, and detect differences between the target sequence and the reference wild gene sequence of the CanIon gene. In one such design, termed 4L tiled array, is implemented a set of four probes (A, C, G, T), preferably 15-nucleotide oligomers. In each set of four probes, the perfect complement will hybridize more strongly than mismatched probes. Consequently, a nucleic acid target of length L is scanned for mutations with a tiled array containing 4L probes, the whole probe set containing all the possible mutations in the known wild reference sequence. The hybridization signals of the 15-mer probe set tiled array are perturbed by a single base change in the target sequence. As a consequence, there is a characteristic loss of signal or a “footprint” for the probes flanking a mutation position. This technique was described by Chee et al. (1996).

Consequently, the invention concerns an array of nucleic acid molecules comprising at least one polynucleotide described above as probes and primers. Preferably, the invention concerns an array of nucleic acid comprising at least two polynucleotides described above as probes and primers.

A further object of the invention consists of an array of nucleic acid sequences comprising either at least one of the sequences selected from the group consisting of P1 to P18, B1 to B17, C1 to C17, D1 to D18, E1 to E18, the sequences complementary thereto, a fragment thereof of at least 8, 10, 12, 15, 18, 20, 25, 30, or 40 consecutive nucleotides thereof, and at least one sequence comprising a biallelic marker selected from the group consisting of A1 to A18 and the complements thereto.

The invention also pertains to an array of nucleic acid sequences comprising either at least two of the sequences selected from the group consisting of P1 to P18, B1 to B17, C1 to C17, D1 to D18, E1 to E18, the sequences complementary thereto, a fragment thereof of at least 8 consecutive nucleotides thereof, and at least two sequences comprising a biallelic marker selected from the group consisting of A1 to A18 and the complements thereof.

### CanIon Proteins and Polypeptide Fragments:

The term “CanIon polypeptides” is used herein to embrace all of the proteins and polypeptides of the present invention. Also forming part of the invention are polypeptides encoded by the polynucleotides of the invention, as well as fusion polypeptides comprising such polypeptides. The invention embodies CanIon proteins from humans, including isolated or purified CanIon proteins consisting of, consisting essentially of, or comprising the sequence of SEQ ID No 5.

The invention concerns the polypeptide encoded by a nucleotide sequence selected from the group consisting of SEQ ID No 1 to 4 and 6, a complementary sequence thereof or a fragment thereto.

The present invention embodies isolated, purified, and recombinant polypeptides comprising a contiguous span of at least 6 amino acids, preferably at least 8 to 10 amino acids, more preferably at least 12, 15, 20, 25, 30, 40, 50, 100, 150, 200, 300, 400, 500, 700, 1000, 1200, 1400, 1600 or 1700 amino acids of SEQ ID No 5. In other preferred embodiments the contiguous stretch of amino acids comprises the site of a mutation or functional mutation, including a deletion, addition, swap or truncation of the amino acids in the CanIon protein sequence.

In preferred embodiments, the invention embodies isolated, purified, and recombinant polypeptides comprising a contiguous span of at least 6 amino acids, preferably at least 8 to 10 amino acids, more preferably at least 12, 15, 20, 25, 30, 40, 50, 100, 150, 200, 300, 400, 500, 700, 1000, 1200, 1400, 1600 or 1700 amino acids of SEQ ID No 5, wherein said contiguous span includes at least 1, 2, 3, 5 or 10 of the amino acid positions 277, 338, 574, 678, 680, 683, 691, 692, 695, 696, 697, 894, 1480, 1481, 1483, 1484, 1485, 1630, 1631, 1632, 1636, 1660, 1667, 1707, 1709 of SEQ ID No 5. Preferably, said contiguous span of SEQ ID No 5 comprises an Alanine residue at position 277; a Serine at position 338; a Valine at position 574; a Leucine at position 678; a Serine at position 680; a Threonine at position 683; a Histidine at position 691; a Serine at position 692; a Serine at position 695; an Alanine at position 696; an Isoleucine at position 697; an Isoleucine at position 894; a Lysine at position 1480; an Arginine at position 1481; a Glycine at position 1483; a Valine at position 1484; an Isoleucine at position 1485; an Asparagine at position 1630; a Serine at position 1631; a Methionine at position 1632; a Threonine at position 1636; an Alanine at position 1660; a Phenylalanine at position 1667; a Threonine at position 1707; and/or an Alanine at position 1709. Polynucleotides encoding any of these polypeptides are also provided.

The invention also encompasses a purified, isolated, or recombinant polypeptides comprising an amino acid sequence having at least 70, 75, 80, 85, 90, 95, 98 or 99% amino acid identity with the amino acid sequence of SEQ ID No 5 or a fragment thereof.

CanIon proteins are preferably isolated from human or mammalian tissue samples or expressed from human or mammalian genes. The CanIon polypeptides of the invention can be made using routine expression methods known in the art. The polynucleotide encoding the desired polypeptide, is ligated into an expression vector suitable for any convenient host. Both eukaryotic and prokaryotic host systems is used in forming recombinant polypeptides, and a summary of some of the more common systems. The polypeptide is then isolated from lysed cells or from the culture medium and purified to the extent needed for its intended use. Purification is by any technique known in the art, for example, differential extraction, salt fractionation, chromatography, centrifugation, and the like. See, for example, Methods in Enzymology for a variety of methods for purifying proteins.

In addition, shorter protein fragments is produced by chemical synthesis. Alternatively the proteins of the invention is extracted from cells or tissues of humans or non-human animals. Methods for purifying proteins are known in the art, and include the use of detergents or chaotropic agents to disrupt particles followed by differential extraction and separation of the polypeptides by ion exchange chromatography, affinity chromatography, sedimentation according to density, and gel electrophoresis.

Any CanIon cDNA, including SEQ ID No 4, may be used to express CanIon proteins and polypeptides. The nucleic acid encoding the CanIon protein or polypeptide to be expressed is operably linked to a promoter in an expression vector using conventional cloning technology. The CanIon insert in the expression vector may comprise the full coding sequence for the CanIon protein or a portion thereof.

The expression vector is any of the mammalian, yeast, insect or bacterial expression systems known in the art. Commercially available vectors and expression systems are available from a variety of suppliers including Genetics Institute (Cambridge, Mass.), Stratagene (La Jolla, Calif.), Promega (Madison, Wis.), and Invitrogen (San Diego, Calif.). If desired, to enhance expression and facilitate proper protein folding, the codon context and codon pairing of the sequence is optimized for the particular expression organism in which the expression vector is introduced, as explained by Hatfield, et al., U.S. Pat. No. 5,082,767, the disclosures of which are incorporated by reference herein in their entirety.

In one embodiment, the entire coding sequence of the CanIon cDNA through the polyA signal of the cDNA are operably linked to a promoter in the expression vector. Alternatively, if the nucleic acid encoding a portion of the CanIon protein lacks a methionine to serve as the initiation site, an initiating methionine can be introduced next to the first codon of the nucleic acid using conventional techniques. Similarly, if the insert from the CanIon cDNA lacks a polyA signal, this sequence can be added to the construct by, for example, splicing out the PolyA signal from pSG5 (Stratagene) using BglI and SalI restriction endonuclease enzymes and incorporating it into the mammalian expression vector pXT1 (Stratagene). pXT1 contains the LTRs and a portion of the gag gene from Moloney Murine Leukemia Virus. The position of the LTRs in the construct allow efficient stable transfection. The vector includes the Herpes Simplex Thymidine Kinase promoter and the selectable neomycin gene. The nucleic acid encoding the CanIon protein or a portion thereof is obtained by PCR from a bacterial vector containing the CanIon cDNA of SEQ ID No 5 using oligonucleotide primers complementary to the CanIon cDNA or portion thereof and containing restriction endonuclease sequences for Pst I incorporated into the 5′ primer and BglII at the 5′ end of the corresponding cDNA 3′ primer, taking care to ensure that the sequence encoding the CanIon protein or a portion thereof is positioned properly with respect to the polyA signal. The purified fragment obtained from the resulting PCR reaction is digested with PstI, blunt ended with an exonuclease, digested with Bgl II, purified and ligated to pXT1, now containing a poly A signal and digested with BglII.

The ligated product may be transfected into mouse NIH 3T3 cells using Lipofectin (Life Technologies, Inc., Grand Island, N.Y.) under conditions outlined in the product specification. Positive transfectants are selected after growing the transfected cells in 600 ug/ml G418 (Sigma, St. Louis, Mo.).

The above procedures may also be used to express a mutant CanIon protein responsible for a detectable phenotype or a portion thereof.

The expressed protein may be purified using conventional purification techniques such as ammonium sulfate precipitation or chromatographic separation based on size or charge. The protein encoded by the nucleic acid insert may also be purified using standard immunochromatography techniques. In such procedures, a solution containing the expressed CanIon protein or portion thereof, such as a cell extract, is applied to a column having antibodies against the CanIon protein or portion thereof is attached to the chromatography matrix. The expressed protein is allowed to bind the immunochromatography column. Thereafter, the column is washed to remove non-specifically bound proteins. The specifically bound expressed protein is then released from the column and recovered using standard techniques.

To confirm expression of the CanIon protein or a portion thereof, the proteins expressed from host cells containing an expression vector containing an insert encoding the CanIon protein or a portion thereof can be compared to the proteins expressed in host cells containing the expression vector without an insert. The presence of a band in samples from cells containing the expression vector with an insert which is absent in samples from cells containing the expression vector without an insert indicates that the CanIon protein or a portion thereof is being expressed. Generally, the band will have the mobility expected for the CanIon protein or portion thereof. However, the band may have a mobility different than that expected as a result of modifications such as glycosylation, ubiquitination, or enzymatic cleavage.

Antibodies capable of specifically recognizing the expressed CanIon protein or a portion thereof are described below.

If antibody production is not possible, the nucleic acids encoding the CanIon protein or a portion thereof is incorporated into expression vectors designed for use in purification schemes employing chimeric polypeptides. In such strategies the nucleic acid encoding the CanIon protein or a portion thereof is inserted in frame with the gene encoding the other half of the chimera. The other half of the chimera is β-globin or a nickel binding polypeptide encoding sequence. A chromatography matrix having antibody to β-globin or nickel attached thereto is then used to purify the chimeric protein. Protease cleavage sites is engineered between the β-globin gene or the nickel binding polypeptide and the CanIon protein or portion thereof. Thus, the two polypeptides of the chimera is separated from one another by protease digestion.

One useful expression vector for generating β-globin chimeric proteins is pSG5 (Stratagene), which encodes rabbit β-globin. Intron II of the rabbit β-globin gene facilitates splicing of the expressed transcript, and the polyadenylation signal incorporated into the construct increases the level of expression. These techniques are well known to those skilled in the art of molecular biology. Standard methods are published in methods texts such as Davis et al. (1986) and many of the methods are available from Stratagene, Life Technologies, Inc., or Promega. Polypeptide may additionally be produced from the construct using in vitro translation systems such as the In vitro Express™ Translation Kit (Stratagene).

Antibodies that Bind CanIon Polypeptides of the Invention

Any CanIon polypeptide or whole protein may be used to generate antibodies capable of specifically binding to an expressed CanIon protein or fragments thereof as described.

One antibody composition of the invention is capable of specifically or selectively binding to the variant of the CanIon protein of SEQ ID No 5. For an antibody composition to specifically bind to a first variant of CanIon, it must demonstrate at least a 5%, 10%, 15%, 20%, 25%, 50%, or 100% greater binding affinity for a full length first variant of the CanIon protein than for a full length second variant of the CanIon protein in an ELISA, RIA, or other antibody-based binding assay. In a preferred embodiment an antibody composition is capable of specifically binding a human CanIon protein.

In a preferred embodiment, the invention concerns antibody compositions, either polyclonal or monoclonal, capable of selectively binding, or selectively bind to an epitope-containing a polypeptide comprising a contiguous span of at least 6 amino acids, preferably at least 8 to 10 amino acids, more preferably at least 12, 15, 20, 25, 30, 40, 50, 100, 200, 300, 400, 500, 700 or 1000 amino acids of SEQ ID No 5. In preferred embodiments, said contiguous span includes at least 1, 2, 3, 5 or 10 of the amino acid positions 277, 338, 574, 678, 680, 683, 691, 692, 695, 696, 697, 894, 1480, 1481, 1483, 1484, 1485, 1630, 1631, 1632, 1636, 1660, 1667, 1707, 1709 of SEQ ID No 5.

Any CanIon polypeptide or whole protein may be used to generate antibodies capable of specifically binding to an expressed CanIon protein or fragments thereof as described.

An epitope can comprise as few as 3 amino acids in a spatial conformation, which is unique to the epitope. Generally an epitope consists of at least 6 such amino acids, and more often at least 8-10 such amino acids. In preferred embodiment, antigenic epitopes comprise a number of amino acids that is any integer between 3 and 50. Fragments which function as epitopes may be produced by any conventional means. Epitopes can be determined by a Jameson-Wolf antigenic analysis, for example, performed using the computer program PROTEAN, using default parameters (Version 4.0 Windows, DNASTAR, Inc., 1228 South Park Street Madison, Wis.

The invention also concerns a purified or isolated antibody capable of specifically binding to a mutated CanIon protein or to a fragment or variant thereof comprising an epitope of the mutated CanIon protein. In another preferred embodiment, the present invention concerns an antibody capable of binding to a polypeptide comprising at least 10 consecutive amino acids of a CanIon protein and including at least one of the amino acids which can be encoded by the trait causing mutations.

Non-human animals or mammals, whether wild-type or transgenic, which express a different species of CanIon than the one to which antibody binding is desired, and animals which do not express CanIon (i.e. a CanIon knock out animal as described herein) are particularly useful for preparing antibodies. CanIon knock out animals will recognize all or most of the exposed regions of a CanIon protein as foreign antigens, and therefore produce antibodies with a wider array of CanIon epitopes. Moreover, smaller polypeptides with only 10 to 30 amino acids may be useful in obtaining specific binding to any one of the CanIon proteins. In addition, the humoral immune system of animals which produce a species of CanIon that resembles the antigenic sequence will preferentially recognize the differences between the animal's native CanIon species and the antigen sequence, and produce antibodies to these unique sites in the antigen sequence. Such a technique will be particularly useful in obtaining antibodies that specifically bind to any one of the CanIon proteins.

Antibody preparations prepared according to either protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

The antibodies of the invention may be labeled by any one of the radioactive, fluorescent or enzymatic labels known in the art.

Consequently, the invention is also directed to a method for detecting specifically the presence of a CanIon polypeptide according to the invention in a biological sample, said method comprising the following steps:

a) bringing into contact the biological sample with a polyclonal or monoclonal antibody that specifically binds a CanIon polypeptide comprising an amino acid sequence of SEQ ID No 5, or to a peptide fragment or variant thereof; and

b) detecting the antigen-antibody complex formed.

The invention also concerns a diagnostic kit for detecting in vitro the presence of a CanIon polypeptide according to the present invention in a biological sample, wherein said kit comprises:

a) a polyclonal or monoclonal antibody that specifically binds a CanIon polypeptide comprising an amino acid sequence of SEQ ID No 5, or to a peptide fragment or variant thereof, optionally labeled;

b) a reagent allowing the detection of the antigen-antibody complexes formed, said reagent carrying optionally a label, or being able to be recognized itself by a labeled reagent, more particularly in the case when the above-mentioned monoclonal or polyclonal antibody is not labeled by itself.

The present invention thus relates to antibodies and T-cell antigen receptors (TCR), which specifically bind the polypeptides, and more specifically, the epitopes of the polypeptides of the present invention, including but not limited to IgG (including IgG1, IgG2, IgG3, and IgG4), IgA (including IgA1 and IgA2), IgD, IgE, or IgM, and IgY. In a preferred embodiment the antibodies are human antigen binding antibody fragments of the present invention include, but are not limited to, Fab, Fab′F(ab)₂ and F(ab′)2, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are human, murine, rabbit, goat, guinea pig, camel, horse, or chicken.

Antigen-binding antibody fragments, including single-chain antibodies, may comprise the variable region(s) alone or in combination with the entire or partial of the following: hinge region, CH1, CH2, and CH3 domains. Also included in the invention are any combinations of variable region(s) and hinge region, CH1, CH2, and CH3 domains. The present invention further includes chimeric, humanized, and human monoclonal and polyclonal antibodies, which specifically bind the polypeptides of the present invention. The present invention further includes antibodies that are anti-idiotypic to the antibodies of the present invention.

The antibodies of the present invention may be monospecific, bispecific, trispecific or have greater multispecificity. Multispecific antibodies may be specific for different epitopes of a polypeptide of the present invention or may be specific for both a polypeptide of the present invention as well as for heterologous compositions, such as a heterologous polypeptide or solid support material. See, e.g., WO 93/17715; WO 92/08802; WO 91/00360; WO 92/05793; Tutt, et al. (1991) J. Immunol. 147:60-69; U.S. Pat. Nos. 5,573,920, 4,474,893, 5,601,819, 4,714,681, 4,925,648; Kostelny, et al. (1992) J. Immunol. 148:1547-1553.

Antibodies of the present invention may be described or specified in terms of the epitope(s) or epitope-bearing portion(s) of a polypeptide of the present invention, which are recognized or specifically bound by the antibody. In the case of proteins of the present invention secreted proteins, the antibodies may specifically bind a full-length protein encoded by a nucleic acid of the present invention, a mature protein (i.e., the protein generated by cleavage of the signal peptide) encoded by a nucleic acid of the present invention, a signal peptide encoded by a nucleic acid of the present invention, or any other polypeptide of the present invention. Therefore, the epitope(s) or epitope bearing polypeptide portion(s) may be specified as described herein, e.g., by N-terminal and C-terminal positions, by size in contiguous amino acid residues, or otherwise described herein (including the sequence listing). Antibodies which specifically bind any epitope or polypeptide of the present invention may also be excluded as individual species. Therefore, the present invention includes antibodies that specifically bind specified polypeptides of the present invention, and allows for the exclusion of the same.

Antibodies of the present invention may also be described or specified in terms of their cross-reactivity. Antibodies that do not specifically bind any other analog, ortholog, or homolog of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. Further included in the present invention are antibodies, which only bind polypeptides encoded by polynucleotides, which hybridize to a polynucleotide of the present invention under stringent hybridization conditions (as described herein). Antibodies of the present invention may also be described or specified in terms of their binding affinity. Preferred binding affinities include those with a dissociation constant or Kd less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰ M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻⁵M, and 10⁻⁵M.

Antibodies of the present invention have uses that include, but are not limited to, methods known in the art to purify, detect, and target the polypeptides of the present invention including both in vitro and in vivo diagnostic and therapeutic methods. For example, the antibodies have use in immunoassays for qualitatively and quantitatively measuring levels of the polypeptides of the present invention in biological samples. See, e.g., Harlow et al., ANTIBODIES: A LABORATORY MANUAL, (Cold Spring Harbor Laboratory Press, 2nd ed. 1988) (incorporated by reference in its entirety).

The antibodies of the present invention may be used either alone or in combination with other compositions. The antibodies may further be recombinantly fused to a heterologous polypeptide at the N- or C-terminus or chemically conjugated (including covalent and non-covalent conjugations) to polypeptides or other compositions. For example, antibodies of the present invention may be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

The antibodies of the present invention may be prepared by any suitable method known in the art. For example, a polypeptide of the present invention or an antigenic fragment thereof can be administered to an animal in order to induce the production of sera containing polyclonal antibodies. The term “monoclonal antibody” is not limited to antibodies produced through hybridoma technology. The term “antibody” refers to a polypeptide or group of polypeptides which are comprised of at least one binding domain, where a binding domain is formed from the folding of variable domains of an antibody molecule to form three-dimensional binding spaces with an internal surface shape and charge distribution complementary to the features of an antigenic determinant of an antigen., which allows an immunological reaction with the antigen. The term “monoclonal antibody” refers to an antibody that is derived from a single clone, including eukaryotic, prokaryotic, or phage clone, and not the method by which it is produced. Monoclonal antibodies can be prepared using a wide variety of techniques known in the art including the use of hybridoma, recombinant, and phage display technology.

Hybridoma techniques include those known in the art (see, e.g., Harlow et al. (1998); Hammerling, et al. (1981) (said references incorporated by reference in their entireties). Fab and F(ab′)2 fragments may be produced, for example, from hybridoma-produced antibodies by proteolytic cleavage, using enzymes such as papain (to produce Fab fragments) or pepsin (to produce F(ab′)2 fragments).

Alternatively, antibodies of the present invention can be produced through the application of recombinant DNA technology or through synthetic chemistry using methods known in the art. For example, the antibodies of the present invention can be prepared using various phage display methods known in the art. In phage display methods, functional antibody domains are displayed on the surface of a phage particle, which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene VIII protein. Examples of phage display methods that can be used to make the antibodies of the present invention include those disclosed in Brinkman, et al. (1995); Ames, et al. (1995); Kettleborough, et al. (1994); Persic, et al. (1997); Burton, et al. (1994); PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743 (said references incorporated by reference in their entireties).

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′F(ab)₂ and F(ab′)2 fragments can also be employed using methods known in the art such as those disclosed in WO 92/22324; Mullinax, et al. (1992); and Sawai, et al. (1995); and Better, et al. (1988) (said references incorporated by reference in their entireties).

Examples of techniques which can be used to produce single-chain Fvs and antibodies include those described in U.S. Pat. Nos. 4,946,778 and 5,258,498; Huston et al. (1991); Shu, et al. (1993); and Skerra, et al. (1988). For some uses, including in vivo use of antibodies in humans and in vitro detection assays, it may be preferable to use chimeric, humanized, or human antibodies. Methods for producing chimeric antibodies are known in the art. See e.g., Morrison (1985); Oi et al., (1986); Gillies, S. D. et al. (1989); and U.S. Pat. No. 5,807,715. Antibodies can be humanized using a variety of techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., (1991); Studnicka G. M. et al. (1994); Roguska M. A. et al. (1994), and chain shuffling (U.S. Pat. No. 5,565,332). Human antibodies can be made by a variety of methods known in the art including phage display methods described above, See also, U.S. Pat. Nos. 4,444,887, 4,716,111, 5,545,806, and 5,814,318; WO 98/46645; WO 98/50433; WO 98/24893; WO 96/34096; WO 96/33735; and WO 91/10741 (said references incorporated by reference in their entireties).

Further included in the present invention are antibodies recombinantly fused or chemically conjugated (including both covalently and non-covalently conjugations) to a polypeptide of the present invention. The antibodies may be specific for antigens other than polypeptides of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al. supra and WO 93/21232; EP 0 439 095; Naramura, M. et al. (1994); U.S. Pat. No. 5,474,981; Gillies, S. O. et al. (1992); Fell, H. P. et al. (1991) (said references incorporated by reference in their entireties).

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the hinge region, CH1 domain, CH2 domain, and CH3 domain or any combination of whole domains or portions thereof. The polypeptides of the present invention may be fused or conjugated to the above antibody portions to increase the in vivo half-life of the polypeptides or for use in immunoassays using methods known in the art. The polypeptides may also be fused or conjugated to the above antibody portions to form multimers. For example, Fe portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See e.g., U.S. Pat. Nos. 5,336,603, 5,622,929, 5,359,046, 5,349,053, 5,447,851, 5,112,946; EP 0 307 434, EP 0 367 166; WO 96/04388, WO 91/06570; Ashkenazi, A. et al. (1991); Zheng, X. X. et al. (1995); and Vil, H. et al. (1992) (said references incorporated by reference in their entireties).

The invention further relates to antibodies that act as agonists or antagonists of the polypeptides of the present invention. For example, the present invention includes antibodies that disrupt the receptor/ligand interactions with the polypeptides of the invention either partially or fully. Included are both receptor-specific antibodies and ligand-specific antibodies. Included are receptor-specific antibodies, which do not prevent ligand binding but prevent receptor activation. Receptor activation (i.e., signaling) may be determined by techniques described herein or otherwise known in the art. Also include are receptor-specific antibodies which both prevent ligand binding and receptor activation. Likewise, included are neutralizing antibodies that bind the ligand and prevent binding of the ligand to the receptor, as well as antibodies that bind the ligand, thereby preventing receptor activation, but do not prevent the ligand from binding the receptor. Further included are antibodies that activate the receptor. These antibodies may act as agonists for either all or less than all of the biological activities affected by ligand-mediated receptor activation. The antibodies may be specified as agonists or antagonists for biological activities comprising specific activities disclosed herein. The above antibody agonists can be made using methods known in the art. See e.g., WO 96/40281; U.S. Pat. No. 5,811,097; Deng, B. et al. (1998); Chen, Z. et al. (1998); Harrop, J. A. et al., (1998); Zhu, Z. et al. (1998); Yoon, D. Y. et al. (1998); Prat, M. et al. (1998); Pitard, V. et al. (1997); Liautard, J. et al. (1997); Carlson, N. G. et al. (1997); Taryman, R. E. et al. (1995); Muller, Y. A. et al. (1998); Bartunek, P. et al. (1996) (said references incorporated by reference in their entireties).

As discussed above, antibodies of the polypeptides of the invention can, in turn, be utilized to generate anti-idiotypic antibodies that “mimic” polypeptides of the invention using techniques well known to those skilled in the art. See, e.g. Greenspan and Bona, (1989); Nissinoff, (1991). For example, antibodies which bind to and competitively inhibit polypeptide multimerization or binding of a polypeptide of the invention to ligand can be used to generate anti-idiotypes that “mimic” the polypeptide multimerization or binding domain and, as a consequence, bind to and neutralize polypeptide or its ligand. Such neutralization anti-idiotypic antibodies can be used to bind a polypeptide of the invention or to bind its ligands/receptors, and thereby block its biological activity.

### CanIon-Related Biallelic Markers

Advantages of the Biallelic Markers of the Present Invention

The CanIon-related biallelic markers of the present invention offer a number of important advantages over other genetic markers such as RFLP (Restriction fragment length polymorphism) and VNTR (Variable Number of Tandem Repeats) markers.

The first generation of markers, were RFLPs, which are variations that modify the length of a restriction fragment. But methods used to identify and to type RFLPs are relatively wasteful of materials, effort, and time. The second generation of genetic markers were VNTRs, which can be categorized as either minisatellites or microsatellites. Minisatellites are tandemly repeated DNA sequences present in units of 5-50 repeats which are distributed along regions of the human chromosomes ranging from 0.1 to 20 kilobases in length. Since they present many possible alleles, their informative content is very high. Minisatellites are scored by performing Southern blots to identify the number of tandem repeats present in a nucleic acid sample from the individual being tested. However, there are only 10⁴ potential VNTRs that can be typed by Southern blotting. Moreover, both RFLP and VNTR markers are costly and time-consuming to develop and assay in large numbers.

Single nucleotide polymorphism or biallelic markers can be used in the same manner as RFLPs and VNTRs but offer several advantages. SNP are densely spaced in the human genome and represent the most frequent type of variation. An estimated number of more than 107 sites are scattered along the 3×10⁹ base pairs of the human genome. Therefore, SNP occur at a greater frequency and with greater uniformity than RFLP or VNTR markers which means that there is a greater probability that such a marker will be found in close proximity to a genetic locus of interest. SNP are less variable than VNTR markers but are mutationally more stable.

Also, the different forms of a characterized single nucleotide polymorphism, such as the biallelic markers of the present invention, are often easier to distinguish and can therefore be typed easily on a routine basis. Biallelic markers have single nucleotide based alleles and they have only two common alleles, which allows highly parallel detection and automated scoring. The biallelic markers of the present invention offer the possibility of rapid, high throughput genotyping of a large number of individuals.

Biallelic markers are densely spaced in the genome, sufficiently informative and can be assayed in large numbers. The combined effects of these advantages make biallelic markers extremely valuable in genetic studies. Biallelic markers can be used in linkage studies in families, in allele sharing methods, in linkage disequilibrium studies in populations, in association studies of case-control populations or of trait positive and trait negative populations. An important aspect of the present invention is that bialielic markers allow association studies to be performed to identify genes involved in complex traits. Association studies examine the frequency of marker alleles in unrelated case- and control-populations and are generally employed in the detection of polygenic or sporadic traits. Association studies may be conducted within the general population and are not limited to studies performed on related individuals in affected families (linkage studies). Biallelic markers in different genes can be screened in parallel for direct association with disease or response to a treatment. This multiple gene approach is a powerful tool for a variety of human genetic studies as it provides the necessary statistical power to examine the synergistic effect of multiple genetic factors on a particular phenotype, drug response, sporadic trait, or disease state with a complex genetic etiology.

Candidate Gene of the Present Invention

Different approaches can be employed to perform association studies: genome-wide association studies, candidate region association studies and candidate gene association studies. Genome-wide association studies rely on the screening of genetic markers evenly spaced and covering the entire genome. The candidate gene approach is based on the study of genetic markers specifically located in genes potentially involved in a biological pathway related to the trait of interest. In the present invention, CanIon is the candidate gene. The candidate gene analysis clearly provides a short-cut approach to the identification of genes and gene polymorphisms related to a particular trait when some information concerning the biology of the trait is available. However, it should be noted that all of the biallelic markers disclosed in the instant application can be employed as part of genome-wide association studies or as part of candidate region association studies and such uses are specifically contemplated in the present invention and claims.

CanIon-Related Biallelic Markers and Polynucleotides Related Thereto

The invention also concerns CanIon-related biallelic markers. As used herein the term “CanIon-related biallelic marker” relates to a set of biallelic markers in linkage disequilibrium with the CanIon gene. The term CanIon-related biallelic marker includes the biallelic markers designated A1 to A17.

A portion of the biallelic markers of the present invention are disclosed in Table 2. They are also described as a single base polymorphism in the features of in the related SEQ ID Nos 1 to 4 and 6. The pairs of primers allowing the amplification of a nucleic acid containing the polymorphic base of one CanIon biallelic marker are listed in Table 1 of Example 2.

17 CanIon-related biallelic markers, A1 to A17, are located in the genomic sequence of CanIon. Biallelic markers A12 and A16 are located in the exons of CanIon. Biallelic marker A18 is flanking the CanIon gene.

The invention also relates to a purified and/or isolated nucleotide sequence comprising a polymorphic base of a CanIon-related biallelic marker. In preferred embodiments, the biallelic marker is selected from the group consisting of A1 to A18, and the complements thereof. The sequence has between 8 and 1000 nucleotides in length, and preferably comprises at least 8, 10, 12, 15, 18, 20, 25, 35, 40, 50, 60, 70, 80, 100, 250, 500 or 1000 contiguous nucleotides of a nucleotide sequence selected from the group consisting of SEQ ID Nos 1 to 4 and 6 or a variant thereof or a complementary sequence thereto. These nucleotide sequences comprise the polymorphic base of either allele 1 or allele 2 of the considered biallelic marker. Optionally, said biallelic marker may be within 6, 5, 4, 3, 2, or 1 nucleotides of the center of said polynucleotide or at the center of said polynucleotide. Optionally, the 3′ end of said contiguous span may be present at the 3′ end of said polynucleotide. Optionally, biallelic marker may be present at the 3′ end of said polynucleotide. Optionally, said polynucleotide may further comprise a label. Optionally, said polynucleotide can be attached to solid support. In a further embodiment, the polynucleotides defined above can be used alone or in any combination.

The invention also relates to a purified and/or isolated nucleotide sequence comprising between 8 and 1000 contiguous nucleotides, and/or preferably at least 8, 10, 12, 15, 18, 20, 25, 35, 40, 50, 60, 70, 80, 100, 250, 500 or 1000 contiguous nucleotides of a nucleotide sequence selected from the group consisting of SEQ ID Nos 1 to 4 or a variant thereof or a complementary sequence thereto. Optionally, the 3′ end of said polynucleotide may be located within or at least 2, 4, 6, 8, 10, 12, 15, 18, 20, 25, 50, 100, 250, 500, or 1000 nucleotides upstream of a CanIon-related biallelic marker in said sequence. Optionally, said CanIon-related biallelic marker is selected from the group consisting of A1 to A17; Optionally, the 3′ end of said polynucleotide may be located within or at least 2, 4, 6, 8, 10, 12, 15, 18, 20, 25, 50, 100, 250, 500, or 1000 nucleotides upstream of a CanIon-related biallelic marker in said sequence. Optionally, the 3′ end of said polynucleotide may be located 1 nucleotide upstream of a CanIon-related biallelic marker in said sequence. Optionally, said polynucleotide may further comprise a label. Optionally, said polynucleotide can be attached to solid support. In a further embodiment, the polynucleotides defined above can be used alone or in any combination.

In a preferred embodiment, the sequences comprising a polymorphic base of one of the biallelic markers listed in Table 2 are selected from the group consisting of the nucleotide sequences that have a contiguous span of, that consist of, that are comprised in, or that comprises a polynucleotide selected from the group consisting of the nucleic acids of the sequences set forth as the amplicons listed in Table 1 or a variant thereof or a complementary sequence thereto.

The invention further concerns a nucleic acid encoding the CanIon protein, wherein said nucleic acid comprises a polymorphic base of a biallelic marker selected from the group consisting of A12 and A16 and the complements thereof.

The invention also encompasses the use of any polynucleotide for, or any polynucleotide for use in, determining the identity of one or more nucleotides at a CanIon-related biallelic marker. In addition, the polynucleotides of the invention for use in determining the identity of one or more nucleotides at a CanIon-related biallelic marker encompass polynucleotides with any further limitation described in this disclosure, or those following, specified alone or in any combination. Optionally, said CanIon-related biallelic marker is selected from the group consisting of A1 to A18, and the complements thereof, or optionally the biallelic markers in linkage disequilibrium therewith;

optionally, said CanIon-related biallelic marker is selected from the group consisting of A1 to A17, and the complements thereof, or optionally the biallelic markers in linkage disequilibrium therewith; optionally, said CanIon-related biallelic marker is selected from the group consisting of A12 and A16, and the complements thereof, or optionally the biallelic markers in linkage disequilibrium therewith; Optionally, said polynucleotide may comprise a sequence disclosed in the present specification; Optionally, said polynucleotide may consist of, or consist essentially of any polynucleotide described in the present specification; Optionally, said determining may be performed in a hybridization assay, sequencing assay, microsequencing assay, or an enzyme-based mismatch detection assay; Optionally, said polynucleotide may be attached to a solid support, array, or addressable array; Optionally, said polynucleotide may be labeled. A preferred polynucleotide may be used in a hybridization assay for determining the identity of the nucleotide at a CanIon-related biallelic marker. Another preferred polynucleotide may be used in a sequencing or microsequencing assay for determining the identity of the nucleotide at a CanIon-related biallelic marker. A third preferred polynucleotide may be used in an enzyme-based mismatch detection assay for determining the identity of the nucleotide at a CanIon-related biallelic marker. A fourth preferred polynucleotide may be used in amplifying a segment of polynucleotides comprising a CanIon-related biallelic marker. Optionally, any of the polynucleotides described above may be attached to a solid support, array, or addressable array; Optionally, said polynucleotide may be labeled.

Additionally, the invention encompasses the use of any polynucleotide for, or any polynucleotide for use in, amplifying a segment of nucleotides comprising a CanIon-related biallelic marker. In addition, the polynucleotides of the invention for use in amplifying a segment of nucleotides comprising a CanIon-related biallelic marker encompass polynucleotides with any further limitation described in this disclosure, or those following, specified alone or in any combination: Optionally, said CanIon-related biallelic marker is selected from the group consisting of A1 to A18, and the complements thereof, or optionally the biallelic markers in linkage disequilibrium therewith; optionally, said CanIon-related biallelic marker is selected from the group consisting of A1 to A17, and the complements thereof, or optionally the biallelic markers in linkage disequilibrium therewith; optionally, said CanIon-related biallelic marker is selected from the group consisting of A12 and A16, and the complements thereof, or optionally the biallelic markers in linkage disequilibrium therewith; Optionally, said polynucleotide may comprise a sequence disclosed in the present specification; Optionally, said polynucleotide may consist of, or consist essentially of any polynucleotide described in the present specification; Optionally, said amplifying may be performed by PCR or LCR. Optionally, said polynucleotide may be attached to a solid support, array, or addressable array. Optionally, said polynucleotide may be labeled.

The primers for amplification or sequencing reaction of a polynucleotide comprising a biallelic marker of the invention may be designed from the disclosed sequences for any method known in the art. A preferred set of primers are fashioned such that the 3′ end of the contiguous span of identity with a sequence selected from the group consisting of SEQ ID Nos 1 to 4 and 6 or a sequence complementary thereto or a variant thereof is present at the 3′ end of the primer. Such a configuration allows the 3′ end of the primer to hybridize to a selected nucleic acid sequence and dramatically increases the efficiency of the primer for amplification or sequencing reactions. Allele specific primers may be designed such that a polymorphic base of a biallelic marker is at the 3′ end of the contiguous span and the contiguous span is present at the 3′ end of the primer. Such allele specific primers tend to selectively prime an amplification or sequencing reaction so long as they are used with a nucleic acid sample that contains one of the two alleles present at a biallelic marker. The 3′ end of the primer of the invention may be located within or at least 2, 4, 6, 8, 10, 12, 15, 18, 20, 25, 50, 100, 250, 500, or 1000 nucleotides upstream of a CanIon-related biallelic marker in said sequence or at any other location which is appropriate for their intended use in sequencing, amplification or the location of novel sequences or markers. Thus, another set of preferred amplification primers comprise an isolated polynucleotide consisting essentially of a contiguous span of 8 to 50 nucleotides in a sequence selected from the group consisting of SEQ ID Nos 1 to 4 and 6 or a sequence complementary thereto or a variant thereof, wherein the 3′ end of said contiguous span is located at the 3′ end of said polynucleotide, and wherein the 3′end of said polynucleotide is located upstream of a CanIon-related biallelic marker in said sequence. Preferably, those amplification primers comprise a sequence selected from the group consisting of the sequences B1 to B17 and C1 to C17. Primers with their 3′ ends located 1 nucleotide upstream of a biallelic marker of CanIon have a special utility as microsequencing assays. Preferred microsequencing primers are described in Table 4. Optionally, said CanIon-related biallelic marker is selected from the group consisting of A1 to A18, and the complements thereof, or optionally the biallelic markers in linkage disequilibrium therewith; optionally, said CanIon-related biallelic marker is selected from the group consisting of A1 to A17, and the complements thereof, or optionally the biallelic markers in linkage disequilibrium therewith; optionally, said CanIon-related biallelic marker is selected from the group consisting of A 12 and A16, and the complements thereof, or optionally the biallelic markers in linkage disequilibrium therewith; Optionally, microsequencing primers are selected from the group consisting of the nucleotide sequences D1 to D18 and E1 to E18.

The probes of the present invention may be designed from the disclosed sequences for any method known in the art, particularly methods which allow for testing if a marker disclosed herein is present. A preferred set of probes may be designed for use in the hybridization assays of the invention in any manner known in the art such that they selectively bind to one allele of a biallelic marker, but not the other under any particular set of assay conditions. Preferred hybridization probes comprise the polymorphic base of either allele 1 or allele 2 of the considered biallelic marker. Optionally, said biallelic marker may be within 6, 5, 4, 3, 2, or 1 nucleotide(s) of the center of the hybridization probe or at the center of said probe. In a preferred embodiment, the probes are selected from the group consisting of each of the sequences P1 to P18 and each of the complementary sequences thereto.

It should be noted that the polynucleotides of the present invention are not limited to having the exact flanking sequences surrounding the polymorphic bases which are enumerated in Sequence Listing. Rather, it will be appreciated that the flanking sequences surrounding the biallelic markers may be lengthened or shortened to any extent compatible with their intended use and the present invention specifically contemplates such sequences. The flanking regions outside of the contiguous span need not be homologous to native flanking sequences which actually occur in human subjects. The addition of any nucleotide sequence which is compatible with the nucleotides intended use is specifically contemplated.

Primers and probes may be labeled or immobilized on a solid support as described in “Oligonucleotide probes and primers”.

The polynucleotides of the invention which are attached to a solid support encompass polynucleotides with any further limitation described in this disclosure, or those following, specified alone or in any combination: Optionally, said polynucleotides may be specified as attached individually or in groups of at least 2, 5, 8, 10, 12, 15, 20, or 25 distinct polynucleotides of the invention to a single solid support. Optionally, polynucleotides other than those of the invention may attached to the same solid support as polynucleotides of the invention. Optionally, when multiple polynucleotides are attached to a solid support they may be attached at random locations, or in an ordered array. Optionally, said ordered array may be addressable.

The present invention also encompasses diagnostic kits comprising one or more polynucleotides of the invention with a portion or all of the necessary reagents and instructions for genotyping a test subject by determining the identity of a nucleotide at a CanIon-related biallelic marker. The polynucleotides of a kit may optionally be attached to a solid support, or be part of an array or addressable array of polynucleotides. The kit may provide for the determination of the identity of the nucleotide at a marker position by any method known in the art including, but not limited to, a sequencing assay method, a microsequencing assay method, a hybridization assay method, or an enzyme-based mismatch detection assay method.

### Methods for De Novo Identification of Biallelic Markers

Any of a variety of methods can be used to screen a genomic fragment for single nucleotide polymorphisms such as differential hybridization with oligonucleotide probes, detection of changes in the mobility measured by gel electrophoresis or direct sequencing of the amplified nucleic acid. A preferred method for identifying biallelic markers involves comparative sequencing of genomic DNA fragments from an appropriate number of unrelated individuals.

In a first embodiment, DNA samples from unrelated individuals are pooled together, following which the genomic DNA of interest is amplified and sequenced. The nucleotide sequences thus obtained are then analyzed to identify significant polymorphisms. One of the major advantages of this method resides in the fact that the pooling of the DNA samples substantially reduces the number of DNA amplification reactions and sequencing reactions, which must be carried out. Moreover, this method is sufficiently sensitive so that a biallelic marker obtained thereby usually demonstrates a sufficient frequency of its less common allele to be useful in conducting association studies.

In a second embodiment, the DNA samples are not pooled and are therefore amplified and sequenced individually. This method is usually preferred when biallelic markers need to be identified in order to perform association studies within candidate genes. Preferably, highly relevant gene regions such as promoter regions or exon regions may be screened for biallelic markers. A biallelic marker obtained using this method may show a lower degree of informativeness for conducting association studies, e.g. if the frequency of its less frequent allele may be less than about 10%. Such a biallelic marker will, however, be sufficiently informative to conduct association studies and it will further be appreciated that including less informative biallelic markers in the genetic analysis studies of the present invention, may allow in some cases the direct identification of causal mutations, which may, depending on their penetrance, be rare mutations.

The following is a description of the various parameters of a preferred method used by the inventors for the identification of the biallelic markers of the present invention.

Genomic DNA Samples

The genomic DNA samples from which the biallelic markers of the present invention are generated are preferably obtained from unrelated individuals corresponding to a heterogeneous population of known ethnic background. The number of individuals from whom DNA samples are obtained can vary substantially, preferably from about 10 to about 1000, preferably from about 50 to about 200 individuals. It is usually preferred to collect DNA samples from at least about 100 individuals in order to have sufficient polymorphic diversity in a given population to identify as many markers as possible and to generate statistically significant results.

As for the source of the genomic DNA to be subjected to analysis, any test sample can be foreseen without any particular limitation. These test samples include biological samples, which can be tested by the methods of the present invention described herein, and include human and animal body fluids such as whole blood, serum, plasma, cerebrospinal fluid, urine, lymph fluids, and various external secretions of the respiratory, intestinal and genitourinary tracts, tears, saliva, milk, white blood cells, myelomas and the like; biological fluids such as cell culture supernatants; fixed tissue specimens including tumor and non-tumor tissue and lymph node tissues; bone marrow aspirates and fixed cell specimens. The preferred source of genomic DNA used in the present invention is from peripheral venous blood of each donor. Techniques to prepare genomic DNA from biological samples are well known to the skilled technician. Details of a preferred embodiment are provided in Example 1. The person skilled in the art can choose to amplify pooled or unpooled DNA samples.

DNA Amplification

The identification of biallelic markers in a sample of genomic DNA may be facilitated through the use of DNA amplification methods. DNA samples can be pooled or unpooled for the amplification step. DNA amplification techniques are well known to those skilled in the art.

Amplification techniques that can be used in the context of the present invention include, but are not limited to, the ligase chain reaction (LCR) described in EP-A-320 308, WO 9320227 and EP-A-439 182, the polymerase chain reaction (PCR, RT-PCR) and techniques such as the nucleic acid sequence based amplification (NASBA) described in Guatelli J. C., et al. (1990) and in Compton J. (1991), Q-beta amplification as described in European Patent Application No 454-4610, strand displacement amplification as described in Walker et al. (1996) and EP A 684 315 and, target mediated amplification as described in PCT Publication WO 9322461.

LCR and Gap LCR are exponential amplification techniques, both depend on DNA ligase to join adjacent primers annealed to a DNA molecule. In Ligase Chain Reaction (LCR), probe pairs are used which include two primary (first and second) and two secondary (third and fourth) probes, all of which are employed in molar excess to target. The first probe hybridizes to a first segment of the target strand and the second probe hybridizes to a second segment of the target strand, the first and second segments being contiguous so that the primary probes abut one another in 5′ phosphate-3′hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes, which can be ligated to form a complementary, secondary ligated product. It is important to realize that the ligated products are functionally equivalent to either the target or its complement. By repeated cycles of hybridization and ligation, amplification of the target sequence is achieved. A method for multiplex LCR has also been described (WO 9320227). Gap LCR (GLCR) is a version of LCR where the probes are not adjacent but are separated by 2 to 3 bases.

For amplification of mRNAs, it is within the scope of the present invention to reverse transcribe mRNA into cDNA followed by polymerase chain reaction (RT-PCR); or, to use a single enzyme for both steps as described in U.S. Pat. No. 5,322,770 or, to use Asymmetric Gap LCR (RT-AGLCR) as described by Marshall et al. (1994). AGLCR is a modification of GLCR that allows the amplification of RNA.

The PCR technology is the preferred amplification technique used in the present invention. A variety of PCR techniques are familiar to those skilled in the art. For a review of PCR technology, see White (1997) and the publication entitled “PCR Methods and Applications” (1991, Cold Spring Harbor Laboratory Press). In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195; 4,683,202; and 4,965,188, the disclosures of which are incorporated herein by reference in their entireties.

PCR technology is the preferred amplification technique used to identify new biallelic markers. A typical example of a PCR reaction suitable for the purposes of the present invention is provided in Example 2.

One of the aspects of the present invention is a method for the amplification of the human CanIon gene, particularly of a fragment of the genomic sequence of SEQ ID No 1 to 3 or of the cDNA sequence of SEQ ID No 4, or a fragment or a variant thereof in a test sample, preferably using PCR. This method comprises the steps of:
a) contacting a test sample with amplification reaction reagents comprising a pair of amplification primers as described above and located on either side of the polynucleotide region to be amplified, and
b) optionally, detecting the amplification products.

The invention also concerns a kit for the amplification of a CanIon gene sequence, particularly of a portion of the genomic sequence of SEQ ID No 1 to 3 or of the cDNA sequence of SEQ ID No 4, or a variant thereof in a test sample, wherein said kit comprises:
a) a pair of oligonucleotide primers located on either side of the CanIon region to be amplified;
b) optionally, the reagents necessary for performing the amplification reaction.

In one embodiment of the above amplification method and kit, the amplification product is detected by hybridization with a labeled probe having a sequence which is complementary to the amplified region. In another embodiment of the above amplification method and kit, primers comprise a sequence which is selected from the group consisting of the nucleotide sequences of B1 to B17, C1 to C17, D1 to D18, and E1 to E18.

In a first embodiment of the present invention, biallelic markers are identified using genomic sequence information generated by the inventors. Sequenced genomic DNA fragments are used to design primers for the amplification of 500 bp fragments. These 500 bp fragments are amplified from genomic DNA and are scanned for biallelic markers. Primers may be designed using the OSP software (Hillier L. and Green P., 1991). All primers may contain, upstream of the specific target bases, a common oligonucleotide tail that serves as a sequencing primer. Those skilled in the art are familiar with primer extensions, which can be used for these purposes.

Preferred primers, useful for the amplification of genomic sequences encoding the CanIon gene, focus on promoters, exons and splice sites of the genes. A biallelic marker presents a higher probability to be an eventual causal mutation if it is located in these functional regions of the gene. Preferred amplification primers of the invention include the nucleotide sequences B1 to B17 and C1 to C17, detailed further in Example 2, Table 1.

Sequencing of Amplified Genomic DNA and Identification of Single Nucleotide Polymorphisms

The amplification products generated as described above, are then sequenced using any method known and available to the skilled technician. Methods for sequencing DNA using either the dideoxy-mediated method (Sanger method) or the Maxam-Gilbert method are widely known to those of ordinary skill in the art. Such methods are for example disclosed in Sambrook et al. (1989). Alternative approaches include hybridization to high-density DNA probe arrays as described in Chee et al. (1996).

Preferably, the amplified DNA is subjected to automated dideoxy terminator sequencing reactions using a dye-primer cycle sequencing protocol. The products of the sequencing reactions are run on sequencing gels and the sequences are determined using gel image analysis. The polymorphism search is based on the presence of superimposed peaks in the electrophoresis pattern resulting from different bases occurring at the same position. Because each dideoxy terminator is labeled with a different fluorescent molecule, the two peaks corresponding to a biallelic site present distinct colors corresponding to two different nucleotides at the same position on the sequence. However, the presence of two peaks can be an artifact due to background noise. To exclude such an artifact, the two DNA strands are sequenced and a comparison between the peaks is carried out. In order to be registered as a polymorphic sequence, the polymorphism has to be detected on both strands.

The above procedure permits those amplification products, which contain biallelic markers to be identified. The detection limit for the frequency of biallelic polymorphisms detected by sequencing pools of 100 individuals is approximately 0.1 for the minor allele, as verified by sequencing pools of known allelic frequencies. However, more than 90% of the biallelic polymorphisms detected by the pooling method have a frequency for the minor allele higher than 0.25. Therefore, the biallelic markers selected by this method have a frequency of at least 0.1 for the minor allele and less than 0.9 for the major allele. Preferably at least 0.2 for the minor allele and less than 0.8 for the major allele, more preferably at least 0.3 for the minor allele and less than 0.7 for the major allele, thus a heterozygosity rate higher than 0.18, preferably higher than 0.32, more preferably higher than 0.42.

In another embodiment, biallelic markers are detected by sequencing individual DNA samples, the frequency of the minor allele of such a biallelic marker may be less than 0.1.

Validation of the Biallelic Markers of the Present Invention

The polymorphisms are evaluated for their usefulness as genetic markers by validating that both alleles are present in a population. Validation of the biallelic markers is accomplished by genotyping a group of individuals by a method of the invention and demonstrating that both alleles are present. Microsequencing is a preferred method of genotyping alleles. The validation by genotyping step may be performed on individual samples derived from each individual in the group or by genotyping a pooled sample derived from more than one individual. The group can be as small as one individual if that individual is heterozygous for the allele in question. Preferably the group contains at least three individuals, more preferably the group contains five or six individuals, so that a single validation test will be more likely to result in the validation of more of the biallelic markers that are being tested. It should be noted, however, that when the validation test is performed on a small group it may result in a false negative result if as a result of sampling error none of the individuals tested carries one of the two alleles. Thus, the validation process is less useful in demonstrating that a particular initial result is an artifact, than it is at demonstrating that there is a bona fide biallelic marker at a particular position in a sequence. All of the genotyping, haplotyping, association, and interaction study methods of the invention may optionally be performed solely with validated biallelic markers.

Evaluation of the Frequency of the Biallelic Markers of the Present Invention

The validated biallelic markers are further evaluated for their usefulness as genetic markers by determining the frequency of the least common allele at the biallelic marker site. The higher the frequency of the less common allele the greater the usefulness of the biallelic marker is association and interaction studies. The determination of the least common allele is accomplished by genotyping a group of individuals by a method of the invention and demonstrating that both alleles are present. This determination of frequency by genotyping step may be performed on individual samples derived from each individual in the group or by genotyping a pooled sample derived from more than one individual. The group must be large enough to be representative of the population as a whole. Preferably the group contains at least 20 individuals, more preferably the group contains at least 50 individuals, most preferably the group contains at least 100 individuals. Of course the larger the group the greater the accuracy of the frequency determination because of reduced sampling error. A biallelic marker wherein the frequency of the less common allele is 30% or more is termed a “high quality biallelic marker.” All of the genotyping, haplotyping, association, and interaction study methods of the invention may optionally be performed solely with high quality biallelic markers.

Methods for Genotyping an Individual for Biallelic Markers

Methods are provided to genotype a biological sample for one or more biallelic markers of the present invention, all of which may be performed in vitro. Such methods of genotyping comprise determining the identity of a nucleotide at a CanIon biallelic marker site by any method known in the art. These methods find use in genotyping case-control populations in association studies as well as individuals in the context of detection of alleles of biallelic markers which are known to be associated with a given trait, in which case both copies of the biallelic marker present in individual's genome are determined so that an individual may be classified as homozygous or heterozygous for a particular allele.

These genotyping methods can be performed on nucleic acid samples derived from a single individual or pooled DNA samples.

Genotyping can be performed using similar methods as those described above for the identification of the biallelic markers, or using other genotyping methods such as those further described below. In preferred embodiments, the comparison of sequences of amplified genomic fragments from different individuals is used to identify new biallelic markers whereas microsequencing is used for genotyping known biallelic markers in diagnostic and association study applications.

In one embodiment the invention encompasses methods of genotyping comprising determining the identity of a nucleotide at a CanIon-related biallelic marker or the complement thereof in a biological sample; optionally, wherein said CanIon-related biallelic marker is selected from the group consisting of A1 to A18, and the complements thereof, or optionally the biallelic markers in linkage disequilibrium therewith; optionally, wherein said CanIon-related biallelic marker is selected from the group consisting of A1 to A17, and the complements thereof, or optionally the biallelic markers in linkage disequilibrium therewith; optionally, wherein said CanIon-related biallelic marker is selected from the group consisting of A12 and A 16, and the complements thereof, or optionally the biallelic markers in linkage disequilibrium therewith; optionally, wherein said biological sample is derived from a single subject; optionally, wherein the identity of the nucleotides at said biallelic marker is determined for both copies of said biallelic marker present in said individual's genome; optionally, wherein said biological sample is derived from multiple subjects; Optionally, the genotyping methods of the invention encompass methods with any further limitation described in this disclosure, or those following, specified alone or in any combination; Optionally, said method is performed in vitro; optionally, further comprising amplifying a portion of said sequence comprising the biallelic marker prior to said determining step; Optionally, wherein said amplifying is performed by PCR, LCR, or replication of a recombinant vector comprising an origin of replication and said fragment in a host cell; optionally, wherein said determining is performed by a hybridization assay, a sequencing assay, a microsequencing assay, or an enzyme-based mismatch detection assay.

Source of Nucleic Acids for Genotyping

Any source of nucleic acids, in purified or non-purified form, can be utilized as the starting nucleic acid, provided it contains or is suspected of containing the specific nucleic acid sequence desired. DNA or RNA may be extracted from cells, tissues, body fluids and the like as described above. While nucleic acids for use in the genotyping methods of the invention can be derived from any mammalian source, the test subjects and individuals from which nucleic acid samples are taken are generally understood to be human.

Amplification of DNA Fragments Comprising Biallelic Markers

Methods and polynucleotides are provided to amplify a segment of nucleotides comprising one or more biallelic marker of the present invention. It will be appreciated that amplification of DNA fragments comprising biallelic markers may be used in various methods and for various purposes and is not restricted to genotyping. Nevertheless, many genotyping methods, although not all, require the previous amplification of the DNA region carrying the biallelic marker of interest. Such methods specifically increase the concentration or total number of sequences that span the biallelic marker or include that site and sequences located either distal or proximal to it. Diagnostic assays may also rely on amplification of DNA segments carrying a biallelic marker of the present invention. Amplification of DNA may be achieved by any method known in the art. Amplification techniques are described above in the section entitled, “DNA amplification.”

Some of these amplification methods are particularly suited for the detection of single nucleotide polymorphisms and allow the simultaneous amplification of a target sequence and the identification of the polymorphic nucleotide as it is further described below.

The identification of biallelic markers as described above allows the design of appropriate oligonucleotides, which can be used as primers to amplify DNA fragments comprising the biallelic markers of the present invention. Amplification can be performed using the primers initially used to discover new biallelic markers which are described herein or any set of primers allowing the amplification of a DNA fragment comprising a biallelic marker of the present invention.

In some embodiments the present invention provides primers for amplifying a DNA fragment containing one or more biallelic markers of the present invention. Preferred amplification primers are listed in Example 2. It will be appreciated that the primers listed are merely exemplary and that any other set of primers which produce amplification products containing one or more biallelic markers of the present invention are also of use.

The spacing of the primers determines the length of the segment to be amplified. In the context of the present invention, amplified segments carrying biallelic markers can range in size from at least about 25 bp to 35 kbp. Amplification fragments from 25-3000 bp are typical, fragments from 50-1000 bp are preferred and fragments from 100-600 bp are highly preferred. It will be appreciated that amplification primers for the biallelic markers may be any sequence which allow the specific amplification of any DNA fragment carrying the markers. Amplification primers may be labeled or immobilized on a solid support as described in “Oligonucleotide probes and primers”.

Methods of Genotyping DNA samples for Biallelic Markers

Any method known in the art can be used to identify the nucleotide present at a biallelic marker site. Since the biallelic marker allele to be detected has been identified and specified in the present invention, detection will prove simple for one of ordinary skill in the art by employing any of a number of techniques. Many genotyping methods require the previous amplification of the DNA region carrying the biallelic marker of interest. While the amplification of target or signal is often preferred at present, ultrasensitive detection methods which do not require amplification are also encompassed by the present genotyping methods. Methods well-known to those skilled in the art that can be used to detect biallelic polymorphisms include methods such as, conventional dot blot analyzes, single strand conformational polymorphism analysis (SSCP) described by Orita et al. (1989), denaturing gradient gel electrophoresis (DGGE), heteroduplex analysis, mismatch cleavage detection, and other conventional techniques as described in Sheffield et al. (1991), White et al. (1992), Grompe et al. (1989 and 1993). Another method for determining the identity of the nucleotide present at a particular polymorphic site employs a specialized exonuclease-resistant nucleotide derivative as described in U.S. Pat. No. 4,656,127.

Preferred methods involve directly determining the identity of the nucleotide present at a biallelic marker site by sequencing assay, enzyme-based mismatch detection assay, or hybridization assay. The following is a description of some preferred methods. A highly preferred method is the microsequencing technique. The term “sequencing” is generally used herein to refer to polymerase extension of duplex primer/template complexes and includes both traditional sequencing and microsequencing.

### 1) Sequencing Assays

The nucleotide present at a polymorphic site can be determined by sequencing methods. In a preferred embodiment, DNA samples are subjected to PCR amplification before sequencing as described above. DNA sequencing methods are described in “Sequencing Of Amplified Genomic DNA And Identification Of Single Nucleotide Polymorphisms”.

Preferably, the amplified DNA is subjected to automated dideoxy terminator sequencing reactions using a dye-primer cycle sequencing protocol. Sequence analysis allows the identification of the base present at the biallelic marker site.

### 2) Microsequencing Assays

In microsequencing methods, the nucleotide at a polymorphic site in a target DNA is detected by a single nucleotide primer extension reaction. This method involves appropriate microsequencing primers which, hybridize just upstream of the polymorphic base of interest in the target nucleic acid. A polymerase is used to specifically extend the 3′ end of the primer with one single ddNTP (chain terminator) complementary to the nucleotide at the polymorphic site. Next the identity of the incorporated nucleotide is determined in any suitable way.

Typically, microsequencing reactions are carried out using fluorescent ddNTPs and the extended microsequencing primers are analyzed by electrophoresis on ABI 377 sequencing machines to determine the identity of the incorporated nucleotide as described in EP 412 883, the disclosure of which is incorporated herein by reference in its entirety. Alternatively capillary electrophoresis can be used in order to process a higher number of assays simultaneously. An example of a typical microsequencing procedure that can be used in the context of the present invention is provided in Example 4.

Different approaches can be used for the labeling and detection of ddNTPs. A homogeneous phase detection method based on fluorescence resonance energy transfer has been described by Chen and Kwok (1997) and Chen et al. (1997). In this method, amplified genomic DNA fragments containing polymorphic sites are incubated with a 5′-fluorescein-labeled primer in the presence of allelic dye-labeled dideoxyribonucleoside triphosphates and a modified Taq polymerase. The dye-labeled primer is extended one base by the dye-terminator specific for the allele present on the template. At the end of the genotyping reaction, the fluorescence intensities of the two dyes in the reaction mixture are analyzed directly without separation or purification. All these steps can be performed in the same tube and the fluorescence changes can be monitored in real time. Alternatively, the extended primer may be analyzed by MALDI-TOF Mass Spectrometry. The base at the polymorphic site is identified by the mass added onto the microsequencing primer (see Haff and Smimov, 1997).

Microsequencing may be achieved by the established microsequencing method or by developments or derivatives thereof. Alternative methods include several solid-phase microsequencing techniques. The basic microsequencing protocol is the same as described previously, except that the method is conducted as a heterogeneous phase assay, in which the primer or the target molecule is immobilized or captured onto a solid support. To simplify the primer separation and the terminal nucleotide addition analysis, oligonucleotides are attached to solid supports or are modified in such ways that permit affinity separation as well as polymerase extension. The 5′ ends and internal nucleotides of synthetic oligonucleotides can be modified in a number of different ways to permit different affinity separation approaches, e.g., biotinylation. If a single affinity group is used on the oligonucleotides, the oligonucleotides can be separated from the incorporated terminator regent. This eliminates the need of physical or size separation. More than one oligonucleotide can be separated from the terminator reagent and analyzed simultaneously if more than one affinity group is used. This permits the analysis of several nucleic acid species or more nucleic acid sequence information per extension reaction. The affinity group need not be on the priming oligonucleotide but could alternatively be present on the template. For example, immobilization can be carried out via an interaction between biotinylated DNA and streptavidin-coated microtitration wells or avidin-coated polystyrene particles. In the same manner, oligonucleotides or templates may be attached to a solid support in a high-density format. In such solid phase microsequencing reactions, incorporated ddNTPs can be radiolabeled (Syvanen, 1994) or linked to fluorescein (Livak and Hainer, 1994). The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such as p-nitrophenyl phosphate). Other possible reporter-detection pairs include: ddNTP linked to dinitrophenyl (DNP) and anti-DNP alkaline phosphatase conjugate (Harju et al., 1993) or biotinylated ddNTP and horseradish peroxidase-conjugated streptavidin with o-phenylenediamine as a substrate (WO 92/15712, the disclosure of which is incorporated herein by reference in its entirety). As yet another alternative solid-phase microsequencing procedure, Nyren et al. (1993) described a method relying on the detection of DNA polymerase activity by an enzymatic luminometric inorganic pyrophosphate detection assay (ELIDA).

Pastinen et al. (1997) describe a method for multiplex detection of single nucleotide polymorphism in which the solid phase minisequencing principle is applied to an oligonucleotide array format. High-density arrays of DNA probes attached to a solid support (DNA chips) are further described below.

In one aspect the present invention provides polynucleotides and methods to genotype one or more bialielic markers of the present invention by performing a microsequencing assay. Preferred microsequencing primers include the nucleotide sequences D1 to D18 and E1 to E18. It will be appreciated that the microsequencing primers listed in Example 4 are merely exemplary and that, any primer having a 3′ end immediately adjacent to the polymorphic nucleotide may be used. Similarly, it will be appreciated that microsequencing analysis may be performed for any biallelic marker or any combination of biallelic markers of the present invention. One aspect of the present invention is a solid support which includes one or more microsequencing primers listed in Example 4, or fragments comprising at least 8, 12, 15, 20, 25, 30, 40, or 50 consecutive nucleotides thereof, to the extent that such lengths are consistent with the primer described, and having a 3′ terminus immediately upstream of the corresponding biallelic marker, for determining the identity of a nucleotide at a biallelic marker site.

### 3) Mismatch Detection Assays Based on Polymerases and Ligases

In one aspect the present invention provides polynucleotides and methods to determine the allele of one or more biallelic markers of the present invention in a biological sample, by mismatch detection assays based on polymerases and/or ligases. These assays are based on the specificity of polymerases and ligases. Polymerization reactions places particularly stringent requirements on correct base pairing of the 3′ end of the amplification primer and the joining of two oligonucleotides hybridized to a target DNA sequence is quite sensitive to mismatches close to the ligation site, especially at the 3′ end. Methods, primers and various parameters to amplify DNA fragments comprising biallelic markers of the present invention are further described above in “Amplification Of DNA Fragments Comprising Biallelic Markers”.

### Allele Specific Amplification Primers

Discrimination between the two alleles of a biallelic marker can also be achieved by allele specific amplification, a selective strategy, whereby one of the alleles is amplified without amplification of the other allele. For allele specific amplification, at least one member of the pair of primers is sufficiently complementary with a region of a CanIon gene comprising the polymorphic base of a biallelic marker of the present invention to hybridize therewith and to initiate the amplification. Such primers are able to discriminate between the two alleles of a biallelic marker.

This is accomplished by placing the polymorphic base at the 3′ end of one of the amplification primers. Because the extension forms from the 3′ end of the primer, a mismatch at or near this position has an inhibitory effect on amplification. Therefore, under appropriate amplification conditions, these primers only direct amplification on their complementary allele. Determining the precise location of the mismatch and the corresponding assay conditions are well within the ordinary skill in the art.

### Ligation/Amplification Based Methods

The “Oligonucleotide Ligation Assay” (OLA) uses two oligonucleotides which are designed to be capable of hybridizing to abutting sequences of a single strand of a target molecules. One of the oligonucleotides is biotinylated, and the other is detectably labeled. If the precise complementary sequence is found in a target molecule, the oligonucleotides will hybridize such that their termini abut, and create a ligation substrate that can be captured and detected. OLA is capable of detecting single nucleotide polymorphisms and may be advantageously combined with PCR as described by Nickerson et al. (1990). In this method, PCR is used to achieve the exponential amplification of target DNA, which is then detected using OLA.

Other amplification methods which are particularly suited for the detection of single nucleotide polymorphism include LCR (ligase chain reaction), Gap LCR (GLCR) which are described above in “DNA Amplification”. LCR uses two pairs of probes to exponentially amplify a specific target. The sequences of each pair of oligonucleotides, is selected to permit the pair to hybridize to abutting sequences of the same strand of the target. Such hybridization forms a substrate for a template-dependant ligase. In accordance with the present invention, LCR can be performed with oligonucleotides having the proximal and distal sequences of the same strand of a biallelic marker site. In one embodiment, either oligonucleotide will be designed to include the biallelic marker site. In such an embodiment, the reaction conditions are selected such that the oligonucleotides can be ligated together only if the target molecule either contains or lacks the specific nucleotide that is complementary to the biallelic marker on the oligonucleotide. In an alternative embodiment, the oligonucleotides will not include the biallelic marker, such that when they hybridize to the target molecule, a “gap” is created as described in WO 90/01069, the disclosure of which is incorporated herein by reference in its entirety. This gap is then “filled” with complementary dNTPs (as mediated by DNA polymerase), or by an additional pair of oligonucleotides. Thus at the end of each cycle, each single strand has a complement capable of serving as a target during the next cycle and exponential allele-specific amplification of the desired sequence is obtained.

Ligase/Polymerase-mediated Genetic Bit Analysis™ is another method for determining the identity of a nucleotide at a preselected site in a nucleic acid molecule (WO 95/21271). This method involves the incorporation of a nucleoside triphosphate that is complementary to the nucleotide present at the preselected site onto the terminus of a primer molecule, and their subsequent ligation to a second oligonucleotide. The reaction is monitored by detecting a specific label attached to the reaction's solid phase or by detection in solution.

### 4) Hybridization Assay Methods

A preferred method of determining the identity of the nucleotide present at a biallelic marker site involves nucleic acid hybridization. The hybridization probes, which can be conveniently used in such reactions, preferably include the probes defined herein. Any hybridization assay may be used including Southern hybridization, Northern hybridization, dot blot hybridization and solid-phase hybridization (see Sambrook et al., 1989).

Hybridization refers to the formation of a duplex structure by two single stranded nucleic acids due to complementary base pairing. Hybridization can occur between exactly complementary nucleic acid strands or between nucleic acid strands that contain minor regions of mismatch. Specific probes can be designed that hybridize to one form of a biallelic marker and not to the other and therefore are able to discriminate between different allelic forms. Allele-specific probes are often used in pairs, one member of a pair showing perfect match to a target sequence containing the original allele and the other showing a perfect match to the target sequence containing the alternative allele. Hybridization conditions should be sufficiently stringent that there is a significant difference in hybridization intensity between alleles, and preferably an essentially binary response, whereby a probe hybridizes to only one of the alleles. Stringent, sequence specific hybridization conditions, under which a probe will hybridize only to the exactly complementary target sequence are well known in the art (Sambrook et al., 1989). Stringent conditions are sequence dependent and will be different in different circumstances. Generally, stringent conditions are selected to be about 5° C. lower than the thermal melting point (Tm) for the specific sequence at a defined ionic strength and pH. Although such hybridization can be performed in solution, it is preferred to employ a solid-phase hybridization assay. The target DNA comprising a biallelic marker of the present invention may be amplified prior to the hybridization reaction. The presence of a specific allele in the sample is determined by detecting the presence or the absence of stable hybrid duplexes formed between the probe and the target DNA. The detection of hybrid duplexes can be carried out by a number of methods. Various detection assay formats are well known which utilize detectable labels bound to either the target or the probe to enable detection of the hybrid duplexes. Typically, hybridization duplexes are separated from unhybridized nucleic acids and the labels bound to the duplexes are then detected. Those skilled in the art will recognize that wash steps may be employed to wash away excess target DNA or probe as well as unbound conjugate. Further, standard heterogeneous assay formats are suitable for detecting the hybrids using the labels present on the primers and probes.

Two recently developed assays allow hybridization-based allele discrimination with no need for separations or washes (see Landegren U. et al., 1998). The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. Cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time (see Livak et al., 1995). In an alternative homogeneous hybridization based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., 1998).

The polynucleotides provided herein can be used to produce probes which can be used in hybridization assays for the detection of biallelic marker alleles in biological samples. These probes are characterized in that they preferably comprise between 8 and 50 nucleotides, and in that they are sufficiently complementary to a sequence comprising a biallelic marker of the present invention to hybridize thereto and preferably sufficiently specific to be able to discriminate the targeted sequence for only one nucleotide variation. A particularly preferred probe is 25 nucleotides in length. Preferably the biallelic marker is within 4 nucleotides of the center of the polynucleotide probe. In particularly preferred probes, the biallelic marker is at the center of said polynucleotide. Preferred probes comprise a nucleotide sequence selected from the group consisting of amplicons listed in Table 1 and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. Preferred probes comprise a nucleotide sequence selected from the group consisting of P1 to P18 and the sequences complementary thereto. In preferred embodiments the polymorphic base(s) are within 5, 4, 3, 2, 1, nucleotides of the center of the said polynucleotide, more preferably at the center of said polynucleotide.

Preferably the probes of the present invention are labeled or immobilized on a solid support. Labels and solid supports are further described in “Oligonucleotide Probes and Primers”. The probes can be non-extendable as described in “Oligonucleotide Probes and Primers”.

By assaying the hybridization to an allele specific probe, one can detect the presence or absence of a biallelic marker allele in a given sample. High-Throughput parallel hybridization in array format is specifically encompassed within “hybridization assays” and are described below.

### 5) Hybridization to Addressable Arrays of Oligonucleotides

Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (e.g., the chip) at selected positions. Each DNA chip can contain thousands to millions of individual synthetic DNA probes arranged in a grid-like pattern and miniaturized to the size of a dime.

The chip technology has already been applied with success in numerous cases. For example, the screening of mutations has been undertaken in the BRCA1 gene, in S. cerevisiae mutant strains, and in the protease gene of HIV-1 virus (Hacia et al., 1996; Shoemaker et al., 1996; Kozal et al., 1996). Chips of various formats for use in detecting biallelic polymorphisms can be produced on a customized basis by Affymetrix (GeneChip™), Hyseq (HyChip and HyGnostics), and Protogene Laboratories.

In general, these methods employ arrays of oligonucleotide probes that are complementary to target nucleic acid sequence segments from an individual which, target sequences include a polymorphic marker. EP 785280, the disclosure of which is incorporated herein by reference in its entirety, describes a tiling strategy for the detection of single nucleotide polymorphisms. Briefly, arrays may generally be “Tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of nucleotides. Tiling strategies are further described in PCT application No. WO 95/11995. In a particular aspect, arrays are tiled for a number of specific, identified biallelic marker sequences. In particular, the array is tiled to include a number of detection blocks, each detection block being specific for a specific biallelic marker or a set of biallelic markers. For example, a detection block may be tiled to include a number of probes, which span the sequence segment that includes a specific polymorphism. To ensure probes that are complementary to each allele, the probes are synthesized in pairs differing at the biallelic marker. In addition to the probes differing at the polymorphic base, monosubstituted probes are also generally tiled within the detection block. These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the biallelic marker. The monosubstituted probes provide internal controls for the tiled array, to distinguish actual hybridization from artefactual cross-hybridization. Upon completion of hybridization with the target sequence and washing of the array, the array is scanned to determine the position on the array to which the target sequence hybridizes. The hybridization data from the scanned array is then analyzed to identify which allele or alleles of the biallelic marker are present in the sample. Hybridization and scanning may be carried out as described in PCT application No. WO 92/10092 and WO 95/11995 and U.S. Pat. No. 5,424,186.

Thus, in some embodiments, the chips may comprise an array of nucleic acid sequences of fragments of about 15 nucleotides in length. In further embodiments, the chip may comprise an array including at least one of the sequences selected from the group consisting of amplicons listed in table 1 and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. In preferred embodiments the polymorphic base is within 5, 4, 3, 2, 1, nucleotides of the center of the said polynucleotide, more preferably at the center of said polynucleotide. In some embodiments, the chip may comprise an array of at least 2, 3, 4, 5, 6, 7, 8 or more of these polynucleotides of the invention. Solid supports and polynucleotides of the present invention attached to solid supports are further described in “Oligonucleotide Probes And Primers”.

### 6) Integrated Systems

Another technique, which may be used to analyze polymorphisms, includes multicomponent integrated systems, which miniaturize and compartmentalize processes such as PCR and capillary electrophoresis reactions in a single functional device. An example of such technique is disclosed in U.S. Pat. No. 5,589,136, the disclosure of which is incorporated herein by reference in its entirety, which describes the integration of PCR amplification and capillary electrophoresis in chips.

Integrated systems can be envisaged mainly when microfluidic systems are used. These systems comprise a pattern of microchannels designed onto a glass, silicon, quartz, or plastic wafer included on a microchip. The movements of the samples are controlled by electric, electroosmotic or hydrostatic forces applied across different areas of the microchip to create functional microscopic valves and pumps with no moving parts.

For genotyping biallelic markers, the microfluidic system may integrate nucleic acid amplification, microsequencing, capillary electrophoresis and a detection method such as laser-induced fluorescence detection.

### Methods of Genetic Analysis Using the Biallelic Markers of the Present Invention

Different methods are available for the genetic analysis of complex traits (see Lander and Schork, 1994). The search for disease-susceptibility genes is conducted using two main methods: the linkage approach in which evidence is sought for cosegregation between a locus and a putative trait locus using family studies, and the association approach in which evidence is sought for a statistically significant association between an allele and a trait or a trait causing allele (Khoury et al., 1993). In general, the biallelic markers of the present invention find use in any method known in the art to demonstrate a statistically significant correlation between a genotype and a phenotype. The biallelic markers may be used in parametric and non-parametric linkage analysis methods. Preferably, the biallelic markers of the present invention are used to identify genes associated with detectable traits using association studies, an approach which does not require the use of affected families and which permits the identification of genes associated with complex and sporadic traits.

The genetic analysis using the biallelic markers of the present invention may be conducted on any scale. The whole set of biallelic markers of the present invention or any subset of biallelic markers of the present invention corresponding to the candidate gene may be used. Further, any set of genetic markers including a biallelic marker of the present invention may be used. A set of biallelic polymorphisms that could be used as genetic markers in combination with the biallelic markers of the present invention has been described in WO 98/20165. As mentioned above, it should be noted that the biallelic markers of the present invention may be included in any complete or partial genetic map of the human genome. These different uses are specifically contemplated in the present invention and claims.

Linkage Analysis

Linkage analysis is based upon establishing a correlation between the transmission of genetic markers and that of a specific trait throughout generations within a family. Thus, the aim of linkage analysis is to detect marker loci that show cosegregation with a trait of interest in pedigrees.

Parametric Methods

When data are available from successive generations there is the opportunity to study the degree of linkage between pairs of loci. Estimates of the recombination fraction enable loci to be ordered and placed onto a genetic map. With loci that are genetic markers, a genetic map can be established, and then the strength of linkage between markers and traits can be calculated and used to indicate the relative positions of markers and genes affecting those traits (Weir, 1996). The classical method for linkage analysis is the logarithm of odds (lod) score method (see Morton, 1955; Ott, 1991). Calculation of lod scores requires specification of the mode of inheritance for the disease (parametric method). Generally, the length of the candidate region identified using linkage analysis is between 2 and 20 Mb. Once a candidate region is identified as described above, analysis of recombinant individuals using additional markers allows further delineation of the candidate region. Linkage analysis studies have generally relied on the use of a maximum of 5,000 microsatellite markers, thus limiting the maximum theoretical attainable resolution of linkage analysis to about 600 kb on average.

Linkage analysis has been successfully applied to map simple genetic traits that show clear Mendelian inheritance patterns and which have a high penetrance (i.e., the ratio between the number of trait positive carriers of allele a and the total number of a carriers in the population). However, parametric linkage analysis suffers from a variety of drawbacks. First, it is limited by its reliance on the choice of a genetic model suitable for each studied trait. Furthermore, as already mentioned, the resolution attainable using linkage analysis is limited, and complementary studies are required to refine the analysis of the typical 2 Mb to 20 Mb regions initially identified through linkage analysis. In addition, parametric linkage analysis approaches have proven difficult when applied to complex genetic traits, such as those due to the combined action of multiple genes and/or environmental factors. It is very difficult to model these factors adequately in a lod score analysis. In such cases, too large an effort and cost are needed to recruit the adequate number of affected families required for applying linkage analysis to these situations, as recently discussed by Risch and Merikangas (1996).

Non-Parametric Methods

The advantage of the so-called non-parametric methods for linkage analysis is that they do not require specification of the mode of inheritance for the disease, they tend to be more useful for the analysis of complex traits. In non-parametric methods, one tries to prove that the inheritance pattern of a chromosomal region is not consistent with random Mendelian segregation by showing that affected relatives inherit identical copies of the region more often than expected by chance. Affected relatives should show excess “allele sharing” even in the presence of incomplete penetrance and polygenic inheritance. In non-parametric linkage analysis the degree of agreement at a marker locus in two individuals can be measured either by the number of alleles identical by state (IBS) or by the number of alleles identical by descent (IBD). Affected sib pair analysis is a well-known special case and is the simplest form of these methods.

The biallelic markers of the present invention may be used in both parametric and non-parametric linkage analysis. Preferably biallelic markers may be used in non-parametric methods which allow the mapping of genes involved in complex traits. The biallelic markers of the present invention may be used in both IBD- and IBS-methods to map genes affecting a complex trait. In such studies, taking advantage of the high density of biallelic markers, several adjacent biallelic marker loci may be pooled to achieve the efficiency attained by multi-allelic markers (Zhao et al., 1998).

Population Association Studies

The present invention comprises methods for identifying if the CanIon gene is associated with a detectable trait using the biallelic markers of the present invention. In one embodiment the present invention comprises methods to detect an association between a biallelic marker allele or a biallelic marker haplotype and a trait. Further, the invention comprises methods to identify a trait causing allele in linkage disequilibrium with any biallelic marker allele of the present invention.

As described above, alternative approaches can be employed to perform association studies: genome-wide association studies, candidate region association studies and candidate gene association studies. In a preferred embodiment, the biallelic markers of the present invention are used to perform candidate gene association studies. The candidate gene analysis clearly provides a short-cut approach to the identification of genes and gene polymorphisms related to a particular trait when some information concerning the biology of the trait is available. Further, the biallelic markers of the present invention may be incorporated in any map of genetic markers of the human genome in order to perform genome-wide association studies. Methods to generate a high-density map of biallelic markers has been described in U.S. Provisional Patent application Ser. No. 60/082,614. The biallelic markers of the present invention may further be incorporated in any map of a specific candidate region of the genome (a specific chromosome or a specific chromosomal segment for example).

As mentioned above, association studies may be conducted within the general population and are not limited to studies performed on related individuals in affected families. Association studies are extremely valuable as they permit the analysis of sporadic or multifactor traits. Moreover, association studies represent a powerful method for fine-scale mapping enabling much finer mapping of trait causing alleles than linkage studies. Studies based on pedigrees often only narrow the location of the trait causing allele. Association studies using the biallelic markers of the present invention can therefore be used to refine the location of a trait causing allele in a candidate region identified by Linkage Analysis methods. Moreover, once a chromosome segment of interest has been identified, the presence of a candidate gene such as a candidate gene of the present invention, in the region of interest can provide a shortcut to the identification of the trait causing allele. Biallelic markers of the present invention can be used to demonstrate that a candidate gene is associated with a trait. Such uses are specifically contemplated in the present invention.

Determining the Frequency of a Biallelic Marker Allele or of a Biallelic Marker Haplotype in a Population

Association studies explore the relationships among frequencies for sets of alleles between loci.

Determining the Frequency of an Allele in a Population

Allelic frequencies of the biallelic markers in a populations can be determined using one of the methods described above under the heading “Methods for genotyping an individual for biallelic markers”, or any genotyping procedure suitable for this intended purpose. Genotyping pooled samples or individual samples can determine the frequency of a biallelic marker allele in a population. One way to reduce the number of genotypings required is to use pooled samples. A major obstacle in using pooled samples is in terms of accuracy and reproducibility for determining accurate DNA concentrations in setting up the pools. Genotyping individual samples provides higher sensitivity, reproducibility and accuracy and; is the preferred method used in the present invention. Preferably, each individual is genotyped separately and simple gene counting is applied to determine the frequency of an allele of a biallelic marker or of a genotype in a given population.

The invention also relates to methods of estimating the frequency of an allele in a population comprising: a) genotyping individuals from said population for said biallelic marker according to the method of the present invention; b) determining the proportional representation of said biallelic marker in said population. In addition, the methods of estimating the frequency of an allele in a population of the invention encompass methods with any further limitation described in this disclosure, or those following, specified alone or in any combination; optionally, wherein said CanIon-related biallelic marker is selected from the group consisting of A1 to A18, and the complements thereof, or optionally the biallelic markers in linkage disequilibrium therewith; optionally, wherein said CanIon-related biallelic marker is selected from the group consisting of A1 to A17, and the complements thereof, or optionally the biallelic markers in linkage disequilibrium therewith; optionally, wherein said CanIon-related biallelic marker is selected from the group consisting of A12 and A16, and the complements thereof, or optionally the biallelic markers in linkage disequilibrium therewith; Optionally, determining the frequency of a biallelic marker allele in a population may be accomplished by determining the identity of the nucleotides for both copies of said biallelic marker present in the genome of each individual in said population and calculating the proportional representation of said nucleotide at said CanIon-related biallelic marker for the population; Optionally, determining the proportional representation may be accomplished by performing a genotyping method of the invention on a pooled biological sample derived from a representative number of individuals, or each individual, in said population, and calculating the proportional amount of said nucleotide compared with the total.

Determining the Frequency of a Haplotype in a Population

The gametic phase of haplotypes is unknown when diploid individuals are heterozygous at more than one locus. Using genealogical information in families gametic phase can sometimes be inferred (Perlin et al., 1994). When no genealogical information is available different strategies may be used. One possibility is that the multiple-site heterozygous diploids can be eliminated from the analysis, keeping only the homozygotes and the single-site heterozygote individuals, but this approach might lead to a possible bias in the sample composition and the underestimation of low-frequency haplotypes. Another possibility is that single chromosomes can be studied independently, for example, by asymmetric PCR amplification (see Newton et al, 1989; Wu et al., 1989) or by isolation of single chromosome by limit dilution followed by PCR amplification (see Ruano et al., 1990). Further, a sample may be haplotyped for sufficiently close biallelic markers by double PCR amplification of specific alleles (Sarkar, G. and Sommer S. S., 1991). These approaches are not entirely satisfying either because of their technical complexity, the additional cost they entail, their lack of generalization at a large scale, or the possible biases they introduce. To overcome these difficulties, an algorithm to infer the phase of PCR-amplified DNA genotypes introduced by Clark, A. G. (1990) may be used. Briefly, the principle is to start filling a preliminary list of haplotypes present in the sample by examining unambiguous individuals, that is, the complete homozygotes and the single-site heterozygotes. Then other individuals in the same sample are screened for the possible occurrence of previously recognized haplotypes. For each positive identification, the complementary haplotype is added to the list of recognized haplotypes, until the phase information for all individuals is either resolved or identified as unresolved. This method assigns a single haplotype to each multiheterozygous individual, whereas several haplotypes are possible when there are more than one heterozygous site. Alternatively, one can use methods estimating haplotype frequencies in a population without assigning haplotypes to each individual. Preferably, a method based on an expectation-maximization (EM) algorithm (Dempster et al., 1977) leading to maximum-likelihood estimates of haplotype frequencies under the assumption of Hardy-Weinberg proportions (random mating) is used (see Excoffier L. and Slatkin M., 1995). The EM algorithm is a generalized iterative maximum-likelihood approach to estimation that is useful when data are ambiguous and/or incomplete. The EM algorithm is used to resolve heterozygotes into haplotypes. Haplotype estimations are further described below under the heading “Statistical Methods.” Any other method known in the art to determine or to estimate the frequency of a haplotype in a population may be used.

The invention also encompasses methods of estimating the frequency of a haplotype for a set of biallelic markers in a population, comprising the steps of: a) genotyping at least one CanIon-related biallelic marker according to a method of the invention for each individual in said population; b) genotyping a second biallelic marker by determining the identity of the nucleotides at said second biallelic marker for both copies of said second biallelic marker present in the genome of each individual in said population; and c) applying a haplotype determination method to the identities of the nucleotides determined in steps a) and b) to obtain an estimate of said frequency. In addition, the methods of estimating the frequency of a haplotype of the invention encompass methods with any further limitation described in this disclosure, or those following, specified alone or in any combination: optionally, wherein said CanIon-related biallelic marker is selected from the group consisting of A1 to A18, and the complements thereof, or optionally the biallelic markers in linkage disequilibrium therewith; optionally, wherein said CanIon-related biallelic marker is selected from the group consisting of A1 to A17, and the complements thereof, or optionally the biallelic markers in linkage disequilibrium therewith; optionally, wherein said CanIon-related biallelic marker is selected from the group consisting of A12 and A16, and the complements thereof, or optionally the biallelic markers in linkage disequilibrium therewith; Optionally, said haplotype determination method is performed by asymmetric PCR amplification, double PCR amplification of specific alleles, the Clark algorithm, or an expectation-maximization algorithm.

Linkage Disequilibrium Analysis

Linkage disequilibrium is the non-random association of alleles at two or more loci and represents a powerful tool for mapping genes involved in disease traits (see Ajioka R. S. et al., 1997). Biallelic markers, because they are densely spaced in the human genome and can be genotyped in greater numbers than other types of genetic markers (such as RFLP or VNTR markers), are particularly useful in genetic analysis based on linkage disequilibrium.

When a disease mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation: one finds the disease mutation only in the presence of a specific set of marker alleles. Through subsequent generations recombination events occur between the disease mutation and these marker polymorphisms, and the disequilibrium gradually dissipates. The pace of this dissipation is a function of the recombination frequency, so the markers closest to the disease gene will manifest higher levels of disequilibrium than those that are further away. When not broken up by recombination, “ancestral” haplotypes and linkage disequilibrium between marker alleles at different loci can be tracked not only through pedigrees but also through populations. Linkage disequilibrium is usually seen as an association between one specific allele at one locus and another specific allele at a second locus.

The pattern or curve of disequilibrium between disease and marker loci is expected to exhibit a maximum that occurs at the disease locus. Consequently, the amount of linkage disequilibrium between a disease allele and closely linked genetic markers may yield valuable information regarding the location of the disease gene. For fine-scale mapping of a disease locus, it is useful to have some knowledge of the patterns of linkage disequilibrium that exist between markers in the studied region. As mentioned above the mapping resolution achieved through the analysis of linkage disequilibrium is much higher than that of linkage studies. The high density of biallelic markers combined with linkage disequilibrium analysis provides powerful tools for fine-scale mapping. Different methods to calculate linkage disequilibrium are described below under the heading “Statistical Methods”.

Population-Based Case-Control Studies of Trait-Marker Associations

As mentioned above, the occurrence of pairs of specific alleles at different loci on the same chromosome is not random and the deviation from random is called linkage disequilibrium. Association studies focus on population frequencies and rely on the phenomenon of linkage disequilibrium. If a specific allele in a given gene is directly involved in causing a particular trait, its frequency will be statistically increased in an affected (trait positive) population, when compared to the frequency in a trait negative population or in a random control population. As a consequence of the existence of linkage disequilibrium, the frequency of all other alleles present in the haplotype carrying the trait-causing allele will also be increased in trait positive individuals compared to trait negative individuals or random controls. Therefore, association between the trait and any allele (specifically a biallelic marker allele) in linkage disequilibrium with the trait-causing allele will suffice to suggest the presence of a trait-related gene in that particular region. Case-control populations can be genotyped for biallelic markers to identify associations that narrowly locate a trait causing allele. As any marker in linkage disequilibrium with one given marker associated with a trait will be associated with the trait. Linkage disequilibrium allows the relative frequencies in case-control populations of a limited number of genetic polymorphisms (specifically biallelic markers) to be analyzed as an alternative to screening all possible functional polymorphisms in order to find trait-causing alleles. Association studies compare the frequency of marker alleles in unrelated case-control populations, and represent powerful tools for the dissection of complex traits.

Case-Control Populations (Inclusion Criteria)

Population-based association studies do not concern familial inheritance but compare the prevalence of a particular genetic marker, or a set of markers, in case-control populations. They are case-control studies based on comparison of unrelated case (affected or trait positive) individuals and unrelated control (unaffected, trait negative or random) individuals. Preferably the control group is composed of unaffected or trait negative individuals. Further, the control group is ethnically matched to the case population. Moreover, the control group is preferably matched to the case-population for the main known confusion factor for the trait under study (for example age-matched for an age-dependent trait). Ideally, individuals in the two samples are paired in such a way that they are expected to differ only in their disease status. The terms “trait positive population”, “case population” and “affected population” are used interchangeably herein.

An important step in the dissection of complex traits using association studies is the choice of case-control populations (see Lander and Schork, 1994). A major step in the choice of case-control populations is the clinical definition of a given trait or phenotype. Any genetic trait may be analyzed by the association method proposed here by carefully selecting the individuals to be included in the trait positive and trait negative phenotypic groups. Four criteria are often useful: clinical phenotype, age at onset, family history and severity. The selection procedure for continuous or quantitative traits (such as blood pressure for example) involves selecting individuals at opposite ends of the phenotype distribution of the trait under study, so as to include in these trait positive and trait negative populations individuals with non-overlapping phenotypes. Preferably, case-control populations comprise phenotypically homogeneous populations. Trait positive and trait negative populations comprise phenotypically uniform populations of individuals representing each between 1 and 98%, preferably between 1 and 80%, more preferably between 1 and 50%, and more preferably between 1 and 30%, most preferably between 1 and 20% of the total population under study, and preferably selected among individuals exhibiting non-overlapping phenotypes. The clearer the difference between the two trait phenotypes, the greater the probability of detecting an association with biallelic markers. The selection of those drastically different but relatively uniform phenotypes enables efficient comparisons in association studies and the possible detection of marked differences at the genetic level, provided that the sample sizes of the populations under study are significant enough.

In preferred embodiments, a first group of between 50 and 300 trait positive individuals, preferably about 100 individuals, are recruited according to their phenotypes. A similar number of control individuals are included in such studies.

Association Analysis

The invention also comprises methods of detecting an association between a genotype and a phenotype, comprising the steps of: a) determining the frequency of at least one CanIon-related biallelic marker in a trait positive population according to a genotyping method of the invention; b) determining the frequency of said CanIon-related biallelic marker in a control population according to a genotyping method of the invention; and c) determining whether a statistically significant association exists between said genotype and said phenotype. In addition, the methods of detecting an association between a genotype and a phenotype of the invention encompass methods with any further limitation described in this disclosure, or those following, specified alone or in any combination: optionally, wherein said CanIon-related biallelic marker is selected from the group consisting of A1 to A18, and the complements thereof, or optionally the biallelic markers in linkage disequilibrium therewith; optionally, wherein said CanIon-related biallelic marker is selected from the group consisting of A1 to A17, and the complements thereof, or optionally the biallelic markers in linkage disequilibrium therewith; optionally, wherein said CanIon-related biallelic marker is selected from the group consisting of A12 and A16, and the complements thereof, or optionally the biallelic markers in linkage disequilibrium therewith; Optionally, said control population may be a trait negative population, or a random population; Optionally, each of said genotyping steps a) and b) may be performed on a pooled biological sample derived from each of said populations; Optionally, each of said genotyping of steps a) and b) is performed separately on biological samples derived from each individual in said population or a subsample thereof.

The general strategy to perform association studies using biallelic markers derived from a region carrying a candidate gene is to scan two groups of individuals (case-control populations) in order to measure and statistically compare the allele frequencies of the biallelic markers of the present invention in both groups.

If a statistically significant association with a trait is identified for at least one or more of the analyzed biallelic markers, one can assume that: either the associated allele is directly responsible for causing the trait (i.e. the associated allele is the trait causing allele), or more likely the associated allele is in linkage disequilibrium with the trait causing allele. The specific characteristics of the associated allele with respect to the candidate gene function usually give further insight into the relationship between the associated allele and the trait (causal or in linkage disequilibrium). If the evidence indicates that the associated allele within the candidate gene is most probably not the trait causing allele but is in linkage disequilibrium with the real trait causing allele, then the trait causing allele can be found by sequencing the vicinity of the associated marker, and performing further association studies with the polymorphisms that are revealed in an iterative manner.

Association studies are usually run in two successive steps. In a first phase, the frequencies of a reduced number of biallelic markers from the candidate gene are determined in the trait positive and control populations. In a second phase of the analysis, the position of the genetic loci responsible for the given trait is further refined using a higher density of markers from the relevant region. However, if the candidate gene under study is relatively small in length, as is the case for CanIon, a single phase may be sufficient to establish significant associations.

Haplotype Analysis

As described above, when a chromosome carrying a disease allele first appears in a population as a result of either mutation or migration, the mutant allele necessarily resides on a chromosome having a set of linked markers: the ancestral haplotype. This haplotype can be tracked through populations and its statistical association with a given trait can be analyzed. Complementing single point (allelic) association studies with multi-point association studies also called haplotype studies increases the statistical power of association studies. Thus, a haplotype association study allows one to define the frequency and the type of the ancestral carrier haplotype. A haplotype analysis is important in that it increases the statistical power of an analysis involving individual markers.

In a first stage of a haplotype frequency analysis, the frequency of the possible haplotypes based on various combinations of the identified biallelic markers of the invention is determined. The haplotype frequency is then compared for distinct populations of trait positive and control individuals. The number of trait positive individuals, which should be, subjected to this analysis to obtain statistically significant results usually ranges between 30 and 300, with a preferred number of individuals ranging between 50 and 150. The same considerations apply to the number of unaffected individuals (or random control) used in the study. The results of this first analysis provide haplotype frequencies in case-control populations, for each evaluated haplotype frequency a p-value and an odd ratio are calculated. If a statistically significant association is found the relative risk for an individual carrying the given haplotype of being affected with the trait under study can be approximated.

An additional embodiment of the present invention encompasses methods of detecting an association between a haplotype and a phenotype, comprising the steps of: a) estimating the frequency of at least one haplotype in a trait positive population, according to a method of the invention for estimating the frequency of a haplotype; b) estimating the frequency of said haplotype in a control population, according to a method of the invention for estimating the frequency of a haplotype; and c) determining whether a statistically significant association exists between said haplotype and said phenotype. In addition, the methods of detecting an association between a haplotype and a phenotype of the invention encompass methods with any further limitation described in this disclosure, or those following: optionally, wherein said CanIon-related biallelic marker is selected from the group consisting of A1 to A18, and the complements thereof, or optionally the biallelic markers in linkage disequilibrium therewith; optionally, wherein said CanIon-related biallelic marker is selected from the group consisting of A1 to 17, and the complements thereof, or optionally the biallelic markers in linkage disequilibrium therewith; optionally, wherein said CanIon-related biallelic marker is selected from the group consisting of A12 and A16, and the complements thereof, or optionally the biallelic markers in linkage disequilibrium therewith; Optionally, said control population is a trait negative population, or a random population. Optionally, said method comprises the additional steps of determining the phenotype in said trait positive and said control populations prior to step c).

Interaction Analysis

The biallelic markers of the present invention may also be used to identify patterns of biallelic markers associated with detectable traits resulting from polygenic interactions. The analysis of genetic interaction between alleles at unlinked loci requires individual genotyping using the techniques described herein. The analysis of allelic interaction among a selected set of biallelic markers with appropriate level of statistical significance can be considered as a haplotype analysis. Interaction analysis comprises stratifying the case-control populations with respect to a given haplotype for the first loci and performing a haplotype analysis with the second loci with each subpopulation.

Statistical methods used in association studies are further described below.

Testing for Linkage in the Presence of Association

The biallelic markers of the present invention may further be used in TDT (transmission/disequilibrium test). TDT tests for both linkage and association and is not affected by population stratification. TDT requires data for affected individuals and their parents or data from unaffected sibs instead of from parents (see Spielmann S. et al., 1993; Schaid D. J. et al., 1996, Spielmann S, and Ewens W. J., 1998). Such combined tests generally reduce the false-positive errors produced by separate analyses.

### Statistical Methods

In general, any method known in the art to test whether a trait and a genotype show a statistically significant correlation may be used.

1) Methods in Linkage Analysis

Statistical methods and computer programs useful for linkage analysis are well-known to those skilled in the art (see Terwilliger J. D. and Ott J., 1994; Ott J., 1991).

2) Methods to Estimate Haplotype Frequencies in a Population

As described above, when genotypes are scored, it is often not possible to distinguish heterozygotes so that haplotype frequencies cannot be easily inferred. When the gametic phase is not known, haplotype frequencies can be estimated from the multilocus genotypic data. Any method known to person skilled in the art can be used to estimate haplotype frequencies (see Lange K., 1997; Weir, B. S., 1996). Preferably, maximum-likelihood haplotype frequencies are computed using an Expectation-Maximization (EM) algorithm (see Dempster et al., 1977; Excoffier L. and Slatkin M., 1995). This procedure is an iterative process aiming at obtaining maximum-likelihood estimates of haplotype frequencies from multi-locus genotype data when the gametic phase is unknown. Haplotype estimations are usually performed by applying the EM algorithm using for example the EM-HAPLO program (Hawley M. E. et al., 1994) or the Arlequin program (Schneider et al., 1997). The EM algorithm is a generalized iterative maximum likelihood approach to estimation and is briefly described below.

Please note that in the present section, “Methods To Estimate Haplotype Frequencies In A Population,” phenotypes will refer to multi-locus genotypes with unknown haplotypic phase. Genotypes will refer to multi-locus genotypes with known haplotypic phase.

Suppose one has a sample of N unrelated individuals typed for K markers. The data observed are the unknown-phase K-locus phenotypes that can be categorized with F different phenotypes. Further, suppose that we have H possible haplotypes (in the case of K biallelic markers, we have for the maximum number of possible haplotypes H=2^{K}).

For phenotype j with cⱼ possible genotypes, we have:

$${P}_{j} = {\sum }_{i = 1}^{{c}_{j}} P ( genotype ( i ) ) = {\sum }_{i = 1}^{{c}_{j}} P ( {h}_{k} , {h}_{l} ) . \qquad Equation 1$$

where Pⱼ is the probability of the j^{th} phenotype, and P(hₖ,hₗ) is the probability of the i^{th} genotype composed of haplotypes hₖ and hₗ. Under random mating (i.e. Hardy-Weinberg Equilibrium), P(hₖhₗ) is expressed as:

P(hₖ,hₗ)=P(hₖ)² for hₖ=hₗ, and

P(hₖ,hₗ)=2P(hₖ)P(hₗ) for hₖ≠hₗ. Equation 2

The E-M algorithm is composed of the following steps: First, the genotype frequencies are estimated from a set of initial values of haplotype frequencies. These haplotype frequencies are denoted Pₗ⁽⁰⁾, P₂⁽⁰⁾, P₃⁽⁰⁾, . . . , P_{H}⁽⁰⁾. The initial values for the haplotype frequencies may be obtained from a random number generator or in some other way well known in the art. This step is referred to the Expectation step. The next step in the method, called the Maximization step, consists of using the estimates for the genotype frequencies to re-calculate the haplotype frequencies. The first iteration haplotype frequency estimates are denoted by P₁⁽¹⁾, P₂⁽¹⁾, P₃⁽¹⁾, . . . , P_{H}⁽¹⁾. In general, the Expectation step at the s^{th} iteration consists of calculating the probability of placing each phenotype into the different possible genotypes based on the haplotype frequencies of the previous iteration:

$${P ( {h}_{k} , {h}_{l} )}^{( s )} = \frac{{n}_{j}}{N} [ \frac{{{P}_{j} ( {h}_{k} , {h}_{l} )}^{( s )}}{{P}_{j}} ] , \qquad Equation 3$$

where nⱼ is the number of individuals with the j^{th} phenotype and Pⱼ(hₖ,hₗ)^{(s)} is the probability of genotype hₖ,hₗ in phenotype j. In the Maximization step, which is equivalent to the gene-counting method (Smith, Ann. Hum. Genet., 21:254-276, 1957), the haplotype frequencies are re-estimated based on the genotype estimates:

$${P}_{t}^{( s + 1 )} = \frac{1}{2} {\sum }_{j = 1}^{F} {\sum }_{i = 1}^{{c}_{j}} {δ}_{it} {{P}_{j} ( {h}_{k} , {h}_{l} )}^{( s )} . \qquad Equation 4$$

Here, δᵢₜ is an indicator variable which counts the number of occurrences that haplotype t is present in i^{th} genotype; it takes on values 0, 1, and 2.

The E-M iterations cease when the following criterion has been reached. Using Maximum Likelihood Estimation (MLE) theory, one assumes that the phenotypes j are distributed multinomially. At each iteration s, one can compute the likelihood function L. Convergence is achieved when the difference of the log-likehood between two consecutive iterations is less than some small number, preferably 10⁻⁷.

3) Methods to Calculate Linkage Disequilibrium Between Markers

A number of methods can be used to calculate linkage disequilibrium between any two genetic positions, in practice linkage disequilibrium is measured by applying a statistical association test to haplotype data taken from a population.

Linkage disequilibrium between any pair of biallelic markers comprising at least one of the biallelic markers of the present invention (Mᵢ, Mⱼ) having alleles (aᵢ/bᵢ) at marker Mᵢ and alleles (aⱼ/bⱼ) at marker Mⱼ can be calculated for every allele combination (aᵢ,aⱼ; aᵢ,bⱼ; bᵢ,aⱼ and bᵢ,bⱼ), according to the Piazza formula:

Δₐᵢₐⱼ=√θ4−√(θ4+θ3)(θ4+θ2), where:

θ4=−−=frequency of genotypes not having allele aᵢ at Mᵢ and not having allele aⱼ at Mⱼ

θ3=−+=frequency of genotypes not having allele aᵢ at Mᵢ and having allele aⱼ at Mⱼ

θ2+−=frequency of genotypes having allele aᵢ at Mᵢ and not having allele aⱼ at Mⱼ

Linkage disequilibrium (LD) between pairs of biallelic markers (Mᵢ, Mⱼ) can also be calculated for every allele combination (aᵢ,aⱼ; aᵢ,bⱼ; bᵢ,aⱼ and bᵢ,bⱼ), according to the maximum-likelihood estimate (MLE) for delta (the composite genotypic disequilibrium coefficient), as described by Weir (Weir B. S., 1996). The MLE for the composite linkage disequilibrium is:

Dₐᵢₐⱼ=(2n₁+n₂+n₃+n₄/2)/N−2(pr(aᵢ)·pr(aⱼ))

Where nᵢ=Σ phenotype (aᵢ/aᵢ, aⱼ/aⱼ), n₂=Σ phenotype (aᵢ/aᵢ, aⱼ/bⱼ), n₃=Σ phenotype (aᵢ/bᵢ, aⱼ/aⱼ), n4=Σ phenotype (aᵢ/bᵢ, aⱼ/bⱼ) and N is the number of individuals in the sample.

This formula allows linkage disequilibrium between alleles to be estimated when only genotype, and not haplotype, data are available.

Another means of calculating the linkage disequilibrium between markers is as follows. For a couple of biallelic markers, Mᵢ(aᵢ/bᵢ) and Mⱼ(aⱼ/bⱼ), fitting the Hardy-Weinberg equilibrium, one can estimate the four possible haplotype frequencies in a given population according to the approach described above.

The estimation of gametic disequilibrium between ai and aj is simply:

Dₐᵢₐⱼ=pr(haplotype(aᵢ,aⱼ))−pr(aᵢ)·pr(aⱼ).

Where pr(aᵢ) is the probability of allele aᵢ and pr(aⱼ) is the probability of allele aⱼ and where pr(haplotype (aᵢ, aⱼ)) is estimated as in Equation 3 above.

For a couple of biallelic marker only one measure of disequilibrium is necessary to describe the association between Mᵢ and Mⱼ.

Then a normalized value of the above is calculated as follows:

D′ₐᵢₐⱼ=Dₐᵢₐⱼ/max(−pr(aᵢ)·pr(aⱼ),−pr(bᵢ)·pr(bⱼ)) with Dₐᵢₐⱼ<0

D′ₐᵢₐⱼ=Dₐᵢₐⱼ/max(pr(bᵢ)·pr(aⱼ),pr(aᵢ)·pr(bⱼ)) with Dₐᵢₐⱼ>0

The skilled person will readily appreciate that other linkage disequilibrium calculation methods can be used.

Linkage disequilibrium among a set of biallelic markers having an adequate heterozygosity rate can be determined by genotyping between 50 and 1000 unrelated individuals, preferably between 75 and 200, more preferably around 100.

4) Testing for Association

Methods for determining the statistical significance of a correlation between a phenotype and a genotype, in this case an allele at a biallelic marker or a haplotype made up of such alleles, may be determined by any statistical test known in the art and with any accepted threshold of statistical significance being required. The application of particular methods and thresholds of significance are well with in the skill of the ordinary practitioner of the art.

Testing for association is performed by determining the frequency of a biallelic marker allele in case and control populations and comparing these frequencies with a statistical test to determine if their is a statistically significant difference in frequency which would indicate a correlation between the trait and the biallelic marker allele under study. Similarly, a haplotype analysis is performed by estimating the frequencies of all possible haplotypes for a given set of biallelic markers in case and control populations, and comparing these frequencies with a statistical test to determine if their is a statistically significant correlation between the haplotype and the phenotype (trait) under study. Any statistical tool useful to test for a statistically significant association between a genotype and a phenotype may be used. Preferably the statistical test employed is a chi-square test with one degree of freedom. A P-value is calculated (the P-value is the probability that a statistic as large or larger than the observed one would occur by chance).

Statistical Significance

In preferred embodiments, significance for diagnosis purposes, either as a positive basis for further diagnostic tests or as a preliminary starting point for early preventive therapy, the p value related to a biallelic marker association is preferably about 1×10⁻² or less, more preferably about 1×10⁻⁴ or less, for a single biallelic marker analysis and about 1×10⁻³ or less, still more preferably 1×10⁻⁶ or less and most preferably of about 1×10⁻⁸ or less, for a haplotype analysis involving two or more markers. These values are believed to be applicable to any association studies involving single or multiple marker combinations.

The skilled person can use the range of values set forth above as a starting point in order to carry out association studies with biallelic markers of the present invention. In doing so, significant associations between the biallelic markers of the present invention and a trait can be revealed and used for diagnosis and drug screening purposes.

Phenotypic Permutation

In order to confirm the statistical significance of the first stage haplotype analysis described above, it might be suitable to perform further analyses in which genotyping data from case-control individuals are pooled and randomized with respect to the trait phenotype. Each individual genotyping data is randomly allocated to two groups, which contain the same number of individuals as the case-control populations used to compile the data obtained in the first stage. A second stage haplotype analysis is preferably run on these artificial groups, preferably for the markers included in the haplotype of the first stage analysis showing the highest relative risk coefficient. This experiment is reiterated preferably at least between 100 and 10000 times. The repeated iterations allow the determination of the probability to obtain the tested haplotype by chance.

Assessment of Statistical Association

To address the problem of false positives similar analysis may be performed with the same case-control populations in random genomic regions. Results in random regions and the candidate region are compared as described in a co-pending US Provisional Patent Application entitled “Methods, Software And Apparati For Identifying Genomic Regions Harboring A Gene Associated With A Detectable Trait,” U.S. Ser. No. 60/107,986, filed Nov. 10, 1998, the contents of which are incorporated herein by reference.

5) Evaluation of Risk Factors

The association between a risk factor (in genetic epidemiology the risk factor is the presence or the absence of a certain allele or haplotype at marker loci) and a disease is measured by the odds ratio (OR) and by the relative risk (RR). If P(R⁺) is the probability of developing the disease for individuals with R and P(R⁻) is the probability for individuals without the risk factor, then the relative risk is simply the ratio of the two probabilities, that is:

RR=P(R⁺)/P(R⁻)

In case-control studies, direct measures of the relative risk cannot be obtained because of the sampling design. However, the odds ratio allows a good approximation of the relative risk for low-incidence diseases and can be calculated:

$$OR = [ \frac{{F}^{+}}{1 - {F}^{+}} ] / [ \frac{{F}^{-}}{( 1 - {F}^{-} )} ]$$

$$OR = ( {F}^{+} / ( 1 - {F}^{+} ) ) / ( {F}^{-} / ( 1 - {F}^{-} ) )$$

F⁺ is the frequency of the exposure to the risk factor in cases and F— is the frequency of the exposure to the risk factor in controls. F⁺ and F⁻ are calculated using the allelic or haplotype frequencies of the study and further depend on the underlying genetic model (dominant, recessive, additive . . . ).

One can further estimate the attributable risk (AR) which describes the proportion of individuals in a population exhibiting a trait due to a given risk factor. This measure is important in quantifying the role of a specific factor in disease etiology and in terms of the public health impact of a risk factor. The public health relevance of this measure lies in estimating the proportion of cases of disease in the population that could be prevented if the exposure of interest were absent. AR is determined as follows:

AR=P_{E}(RRz−1)/(P_{E}(RR−1)+1)

AR is the risk attributable to a biallelic marker allele or a biallelic marker haplotype. P_{E} is the frequency of exposure to an allele or a haplotype within the population at large; and RR is the relative risk which, is approximated with the odds ratio when the trait under study has a relatively low incidence in the general population.

Identification of Biallelic Markers in Linkage Disequilibrium with the Biallelic Markers of the Invention

Once a first biallelic marker has been identified in a genomic region of interest, the practitioner of ordinary skill in the art, using the teachings of the present invention, can easily identify additional biallelic markers in linkage disequilibrium with this first marker. As mentioned before any marker in linkage disequilibrium with a first marker associated with a trait will be associated with the trait. Therefore, once an association has been demonstrated between a given biallelic marker and a trait, the discovery of additional biallelic markers associated with this trait is of great interest in order to increase the density of biallelic markers in this particular region. The causal gene or mutation will be found in the vicinity of the marker or set of markers showing the highest correlation with the trait.

Identification of additional markers in linkage disequilibrium with a given marker involves: (a) amplifying a genomic fragment comprising a first biallelic marker from a plurality of individuals; (b) identifying of second biallelic markers in the genomic region harboring said first biallelic marker; (c) conducting a linkage disequilibrium analysis between said first biallelic marker and second biallelic markers; and (d) selecting said second biallelic markers as being in linkage disequilibrium with said first marker. Subcombinations comprising steps (b) and (c) are also contemplated.

Methods to identify biallelic markers and to conduct linkage disequilibrium analysis are described herein and can be carried out by the skilled person without undue experimentation. The present invention then also concerns biallelic markers which are in linkage disequilibrium with the biallelic markers A1 to A18 and which are expected to present similar characteristics in terms of their respective association with a given trait.

### Identification of Functional Mutations

Mutations in the CanIon gene which are responsible for a detectable phenotype or trait may be identified by comparing the sequences of the CanIon gene from trait positive and control individuals. Once a positive association is confirmed with a biallelic marker of the present invention, the identified locus can be scanned for mutations. In a preferred embodiment, functional regions such as exons and splice sites, promoters and other regulatory regions of the CanIon gene are scanned for mutations. In a preferred embodiment the sequence of the CanIon gene is compared in trait positive and control individuals. Preferably, trait positive individuals carry the haplotype shown to be associated with the trait and trait negative individuals do not carry the haplotype or allele associated with the trait. The detectable trait or phenotype may comprise a variety of manifestations of altered CanIon function.

The mutation detection procedure is essentially similar to that used for biallelic marker identification. The method used to detect such mutations generally comprises the following steps:
amplification of a region of the CanIon gene comprising a biallelic marker or a group of biallelic markers associated with the trait from DNA samples of trait positive patients and trait-negative controls;
sequencing of the amplified region;
comparison of DNA sequences from trait positive and control individuals;
determination of mutations specific to trait-positive patients.

In one embodiment, said biallelic marker is selected from the group consisting of A1 to A18, and the complements thereof. It is preferred that candidate polymorphisms be then verified by screening a larger population of cases and controls by means of any genotyping procedure such as those described herein, preferably using a microsequencing technique in an individual test format. Polymorphisms are considered as candidate mutations when present in cases and controls at frequencies compatible with the expected association results. Polymorphisms are considered as candidate “trait-causing” mutations when they exhibit a statistically significant correlation with the detectable phenotype.

### Biallelic Markers of the Invention in Methods of Genetic Diagnostics

The CanIon nucleic acid sequence and biallelic markers of the present invention can also be used to develop diagnostic tests capable of identifying individuals who express a detectable trait as the result of a specific genotype or individuals whose genotype places them at risk of developing a detectable trait at a subsequent time. Such a diagnosis can be useful in the staging, monitoring, prognosis and/or prophylactic or curative therapy of numerous diseases or conditions including schizophrenia, bipolar disorder, and other CNS disorders such as epilepsy and pain disorders, cardiovascular conditions such as heart disease, hypertension, arrythmias, and numerous other diseases and conditions.

The diagnostic techniques of the present invention may employ a variety of methodologies to determine whether a test subject has a biallelic marker pattern associated with an increased risk of developing a detectable trait or whether the individual suffers from a detectable trait as a result of a particular mutation, including methods which enable the analysis of individual chromosomes for haplotyping, such as family studies, single sperm DNA analysis or somatic hybrids.

The present invention provides diagnostic methods to determine whether an individual is at risk of developing a disease or suffers from a disease resulting from a mutation or a polymorphism in the CanIon gene. The present invention also provides methods to determine whether an individual has a susceptibility to schizophrenia and bipolar disorder, or to any of the other calcium-channel related conditions known in the art or described herein.

These methods involve obtaining a nucleic acid sample from the individual and, determining, whether the nucleic acid sample contains at least one allele or at least one biallelic marker haplotype, indicative of a risk of developing the trait or indicative that the individual expresses the trait as a result of possessing a particular CanIon polymorphism or mutation (trait-causing allele).

Preferably, in such diagnostic methods, a nucleic acid sample is obtained from the individual and this sample is genotyped using methods described above in “Methods Of Genotyping DNA Samples For Biallelic markers. The diagnostics may be based on a single biallelic marker or a on group of biallelic markers.

In each of these methods, a nucleic acid sample is obtained from the test subject and the biallelic marker pattern of one or more of the biallelic markers A1 to A18 is determined.

In one embodiment, a PCR amplification is conducted on the nucleic acid sample to amplify regions in which polymorphisms associated with a detectable phenotype have been identified. The amplification products are sequenced to determine whether the individual possesses one or more CanIon polymorphisms associated with a detectable phenotype. The primers used to generate amplification products may comprise the primers listed in Table 1. Alternatively, the nucleic acid sample is subjected to microsequencing reactions as described above to determine whether the individual possesses one or more CanIon polymorphisms associated with a detectable phenotype resulting from a mutation or a polymorphism in the CanIon gene. The primers used in the microsequencing reactions may include the primers listed in Table 4. In another embodiment, the nucleic acid sample is contacted with one or more allele specific oligonucleotide probes which, specifically hybridize to one or more CanIon alleles associated with a detectable phenotype. The probes used in the hybridization assay may include the probes listed in Table 3. In another embodiment, the nucleic acid sample is contacted with a second CanIon oligonucleotide capable of producing an amplification product when used with the allele specific oligonucleotide in an amplification reaction. The presence of an amplification product in the amplification reaction indicates that the individual possesses one or more CanIon alleles associated with a detectable phenotype.

In a preferred embodiment the identity of the nucleotide present at, at least one, biallelic marker selected from the group consisting of A1 to A18 and the complements thereof, and the complements thereof, is determined and the detectable trait is schizophrenia and bipolar disorder. Diagnostic kits comprise any of the polynucleotides of the present invention.

These diagnostic methods are extremely valuable as they can, in certain circumstances, be used to initiate preventive treatments or to allow an individual carrying a significant haplotype to foresee warning signs such as minor symptoms.

Diagnostics, which analyze and predict response to a drug or side effects to a drug, may be used to determine whether an individual should be treated with a particular drug. For example, if the diagnostic indicates a likelihood that an individual will respond positively to treatment with a particular drug, the drug may be administered to the individual. Conversely, if the diagnostic indicates that an individual is likely to respond negatively to treatment with a particular drug, an alternative course of treatment may be prescribed. A negative response may be defined as either the absence of an efficacious response or the presence of toxic side effects.

Clinical drug trials represent another application for the markers of the present invention. One or more markers indicative of response to an agent acting against schizophrenia or bipolar disorder or another calcium channel-related condition, or to side effects to an agent acting against schizophrenia or bipolar disorder or another calcium channel-related condition, may be identified using the methods described above. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

In particularly preferred embodiments, the trait analyzed using the present diagnostics is schizophrenia or bipolar disorder. However, the present invention also comprises any of the prevention, diagnostic, prognosis and treatment methods described herein using the biallelic markers of the invention in methods of preventing, diagnosing, managing and treating related disorders, particularly related CNS disorders. By way of example, related disorders may comprise psychotic disorders, mood disorders, autism, substance dependence and alcoholism, pain disorders, epilepsy, mental retardation, and other psychiatric diseases including cognitive, anxiety, eating, impulse-control, and personality disorders, as defined with the Diagnosis and Statistical Manual of Mental Disorders fourth edition (DSM-IV) classification. Other disorders include cardiovascular disorders such as angina, hypertension, or arrythmias.

### Recombinant Vectors

The term “vector” is used herein to designate either a circular or a linear DNA or RNA molecule, which is either double-stranded or single-stranded, and which comprise at least one polynucleotide of interest that is sought to be transferred in a cell host or in a unicellular or multicellular host organism.

The present invention encompasses a family of recombinant vectors that comprise a regulatory polynucleotide derived from the CanIon genomic sequence, and/or a coding polynucleotide from either the CanIon genomic sequence or the cDNA sequence.

Generally, a recombinant vector of the invention may comprise any of the polynucleotides described herein, including regulatory sequences, coding sequences and polynucleotide constructs, as well as any CanIon primer or probe as defined above. More particularly, the recombinant vectors of the present invention can comprise any of the polynucleotides described in the “Genomic Sequences Of The CanIon Gene” section, the “CanIon cDNA Sequences” section, the “Coding Regions” section, the “Polynucleotide constructs” section, and the “Oligonucleotide Probes And Primers” section.

In a first preferred embodiment, a recombinant vector of the invention is used to amplify the inserted polynucleotide derived from a genomic sequence of SEQ ID No 1 to 3 or 6 or a CanIon cDNA, for example the cDNA of SEQ ID No 4 in a suitable cell host, this polynucleotide being amplified every time that the recombinant vector replicates.

A second preferred embodiment of the recombinant vectors according to the invention comprises expression vectors comprising either a regulatory polynucleotide or a coding nucleic acid of the invention, or both. Within certain embodiments, expression vectors are employed to express the CanIon polypeptide which can be then purified and, for example be used in ligand screening assays or as an immunogen in order to raise specific antibodies directed against the CanIon protein. In other embodiments, the expression vectors are used for constructing transgenic animals and also for gene therapy. Expression requires that appropriate signals are provided in the vectors, said signals including various regulatory elements, such as enhancers/promoters from both viral and mammalian sources that drive expression of the genes of interest in host cells. Dominant drug selection markers for establishing permanent, stable cell clones expressing the products are generally included in the expression vectors of the invention, as they are elements that link expression of the drug selection markers to expression of the polypeptide.

More particularly, the present invention relates to expression vectors which include nucleic acids encoding a CanIon protein, preferably the CanIon protein of the amino acid sequence of SEQ ID No 5 or variants or fragments thereof.

The invention also pertains to a recombinant expression vector useful for the expression of the CanIon coding sequence, wherein said vector comprises a nucleic acid of SEQ ID No 4.

Recombinant vectors comprising a nucleic acid containing a CanIon-related biallelic marker is also part of the invention. In a preferred embodiment, said biallelic marker is selected from the group consisting of A1 to A18, and the complements thereof.

Some of the elements which can be found in the vectors of the present invention are described in further detail in the following sections.

The present invention also encompasses primary, secondary, and immortalized homologously recombinant host cells of vertebrate origin, preferably mammalian origin and particularly human origin, that have been engineered to: a) insert exogenous (heterologous) polynucleotides into the endogenous chromosomal DNA of a targeted gene, b) delete endogenous chromosomal DNA, and/or c) replace endogenous chromosomal DNA with exogenous polynucleotides. Insertions, deletions, and/or replacements of polynucleotide sequences may be to the coding sequences of the targeted gene and/or to regulatory regions, such as promoter and enhancer sequences, operably associated with the targeted gene.

The present invention further relates to a method of making a homologously recombinant host cell in vitro or in vivo, wherein the expression of a targeted gene not normally expressed in the cell is altered. Preferably the alteration causes expression of the targeted gene under normal growth conditions or under conditions suitable for producing the polypeptide encoded by the targeted gene. The method comprises the steps of: (a) transfecting the cell in vitro or in vivo with a polynucleotide construct, the polynucleotide construct comprising; (i) a targeting sequence; (ii) a regulatory sequence and/or a coding sequence; and (iii) an unpaired splice donor site, if necessary, thereby producing a transfected cell; and (b) maintaining the transfected cell in vitro or in vivo under conditions appropriate for homologous recombination.

The present invention further relates to a method of altering the expression of a targeted gene in a cell in vitro or in vivo wherein the gene is not normally expressed in the cell, comprising the steps of: (a) transfecting the cell in vitro or in vivo with a polynucleotide construct, the a polynucleotide construct comprising: (i) a targeting sequence; (ii) a regulatory sequence and/or a coding sequence; and (iii) an unpaired splice donor site, if necessary, thereby producing a transfected cell; and (b) maintaining the transfected cell in vitro or in vivo under conditions appropriate for homologous recombination, thereby producing a homologously recombinant cell; and (c) maintaining the homologously recombinant cell in vitro or in vivo under conditions appropriate for expression of the gene.

The present invention further relates to a method of making a polypeptide of the present invention by altering the expression of a targeted endogenous gene in a cell in vitro or in vivo wherein the gene is not normally expressed in the cell, comprising the steps of: a) transfecting the cell in vitro with a polynucleotide construct, the a polynucleotide construct comprising: (i) a targeting sequence; (ii) a regulatory sequence and/or a coding sequence; and (iii) an unpaired splice donor site, if necessary, thereby producing a transfected cell; (b) maintaining the transfected cell in vitro or in vivo under conditions appropriate for homologous recombination, thereby producing a homologously recombinant cell; and c) maintaining the homologously recombinant cell in vitro or in vivo under conditions appropriate for expression of the gene, thereby making the polypeptide.

The present invention further relates to a polynucleotide construct which alters the expression of a targeted gene in a cell type in which the gene is not normally expressed. This occurs when the polynucleotide construct is inserted into the chromosomal DNA of the target cell, wherein the a polynucleotide construct comprises: a) a targeting sequence; b) a regulatory sequence and/or coding sequence; and c) an unpaired splice-donor site, if necessary. Further included are a polynucleotide constructs, as described above, wherein the construct further comprises a polynucleotide which encodes a polypeptide and is in-frame with the targeted endogenous gene after homologous recombination with chromosomal DNA.

The compositions may be produced, and methods performed, by techniques known in the art, such as those described in U.S. Pat. Nos. 6,054,288; 6,048,729; 6,048,724; 6,048,524; 5,994,127; 5,968,502; 5,965,125; 5,869,239; 5,817,789; 5,783,385; 5,733,761; 5,641,670; 5,580,734; International Publication Nos: WO96/29411, WO 94/12650; and scientific articles including Koller et al., PNAS 86:8932-8935 (1989).

1. General Features of the Expression Vectors of the Invention

A recombinant vector according to the invention comprises, but is not limited to, a YAC (Yeast Artificial Chromosome), a BAC (Bacterial Artificial Chromosome), a phage, a phagemid, a cosmid, a plasmid or even a linear DNA molecule which may comprise a chromosomal, non-chromosomal, semi-synthetic and synthetic DNA. Such a recombinant vector can comprise a transcriptional unit comprising an assembly of:

(1) a genetic element or elements having a regulatory role in gene expression, for example promoters or enhancers. Enhancers are cis-acting elements of DNA, usually from about 10 to 300 bp in length that act on the promoter to increase the transcription.

(2) a structural or coding sequence which is transcribed into mRNA and eventually translated into a polypeptide, said structural or coding sequence being operably linked to the regulatory elements described in (1); and

(3) appropriate transcription initiation and termination sequences. Structural units intended for use in yeast or eukaryotic expression systems preferably include a leader sequence enabling extracellular secretion of translated protein by a host cell. Alternatively, when a recombinant protein is expressed without a leader or transport sequence, it may include a N-terminal residue. This residue may or may not be subsequently cleaved from the expressed recombinant protein to provide a final product.

Generally, recombinant expression vectors will include origins of replication, selectable markers permitting transformation of the host cell, and a promoter derived from a highly expressed gene to direct transcription of a downstream structural sequence. The heterologous structural sequence is assembled in appropriate phase with translation initiation and termination sequences, and preferably a leader sequence capable of directing secretion of the translated protein into the periplasmic space or the extracellular medium. In a specific embodiment wherein the vector is adapted for transfecting and expressing desired sequences in mammalian host cells, preferred vectors will comprise an origin of replication in the desired host, a suitable promoter and enhancer, and also any necessary ribosome binding sites, polyadenylation signal, splice donor and acceptor sites, transcriptional termination sequences, and 5′-flanking non-transcribed sequences. DNA sequences derived from the SV40 viral genome, for example SV40 origin, early promoter, enhancer, splice and polyadenylation signals may be used to provide the required non-transcribed genetic elements.

The in vivo expression of a CanIon polypeptide of SEQ ID No 5 or fragments or variants thereof may be useful in order to correct a genetic defect related to the expression of the native gene in a host organism or to the production of a biologically inactive CanIon protein.

Consequently, the present invention also comprises recombinant expression vectors mainly designed for the in vivo production of the CanIon polypeptide of SEQ ID No 5 or fragments or variants thereof by the introduction of the appropriate genetic material in the organism of the patient to be treated. This genetic material may be introduced in vitro in a cell that has been previously extracted from the organism, the modified cell being subsequently reintroduced in the said organism, directly in vivo into the appropriate tissue.

2. Regulatory Elements

Promoters

The suitable promoter regions used in the expression vectors according to the present invention are chosen taking into account the cell host in which the heterologous gene has to be expressed. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell, such as, for example, a human or a viral promoter.

A suitable promoter may be heterologous with respect to the nucleic acid for which it controls the expression or alternatively can be endogenous to the native polynucleotide containing the coding sequence to be expressed. Additionally, the promoter is generally heterologous with respect to the recombinant vector sequences within which the construct promoter/coding sequence has been inserted.

Promoter regions can be selected from any desired gene using, for example, CAT (chloramphenicol transferase) vectors and more preferably pKK232-8 and pCM7 vectors.

Preferred bacterial promoters are the LacI, LacZ, the T3 or T7 bacteriophage RNA polymerase promoters, the gpt, lambda PR, PL and trp promoters (EP 0036776), the polyhedrin promoter, or the p10 protein promoter from baculovirus (Kit Novagen) (Smith et al., 1983; O'Reilly et al., 1992), the lambda PR promoter or also the trc promoter.

Eukaryotic promoters include CMV immediate early, HSV thymidine kinase, early and late SV40, LTRs from retrovirus, and mouse metallothionein-L. Selection of a convenient vector and promoter is well within the level of ordinary skill in the art.

The choice of a promoter is well within the ability of a person skilled in the field of genetic engineering. For example, one may refer to Sambrook et al. (1989) or also to the procedures described by Fuller et al. (1996).

Other Regulatory Elements

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

3. Selectable Markers

Such markers would confer an identifiable change to the cell permitting easy identification of cells containing the expression construct. The selectable marker genes for selection of transformed host cells are preferably dihydrofolate reductase or neomycin resistance for eukaryotic cell culture, TRP1 for S. cerevisiae or tetracycline, rifampicin or ampicillin resistance in E. coli, or levan saccharase for mycobacteria, this latter marker being a negative selection marker.

4. Preferred Vectors.

Bacterial Vectors

As a representative but non-limiting example, useful expression vectors for bacterial use can comprise a selectable marker and a bacterial origin of replication derived from commercially available plasmids comprising genetic elements of pBR322 (ATCC 37017). Such commercial vectors include, for example, pKK223-3 (Pharmacia, Uppsala, Sweden), and GEMI (Promega Biotec, Madison, Wis., USA).

Large numbers of other suitable vectors are known to those of skill in the art, and commercially available, such as the following bacterial vectors: pQE70, pQE60, pQE-9 (Qiagen), pbs, pD10, phagescript, psiX174, pbluescript SK, pbsks, pNH8A, pNH16A, pNH18A, pNH46A (Stratagene); ptrc99a, pKK223-3, pKK233-3, pDR540, pRIT5 (Pharmacia); pWLNEO, pSV2CAT, pOG44, pXT1, pSG (Stratagene); pSVK3, pBPV, pMSG, pSVL (Pharmacia); pQE-30 (QIAexpress).

Bacteriophage Vectors

The P1 bacteriophage vector may contain large inserts ranging from about 80 to about 100 kb.

The construction of P1 bacteriophage vectors such as p158 or p158/neo8 are notably described by Sternberg (1992, 1994). Recombinant P1 clones comprising CanIon nucleotide sequences may be designed for inserting large polynucleotides of more than 40 kb (Linton et al., 1993). To generate P1 DNA for transgenic experiments, a preferred protocol is the protocol described by McCormick et al. (1994). Briefly, E. coli (preferably strain NS3529) harboring the P1 plasmid are grown overnight in a suitable broth medium containing 25 μg/ml of kanamycin. The P1 DNA is prepared from the E. coli by alkaline lysis using the Qiagen Plasmid Maxi kit (Qiagen, Chatsworth, Calif., USA), according to the manufacturer's instructions. The P1 DNA is purified from the bacterial lysate on two Qiagen-tip 500 columns, using the washing and elution buffers contained in the kit. A phenol/chloroform extraction is then performed before precipitating the DNA with 70% ethanol. After solubilizing the DNA in TE (10 mM Tris-HCl, pH 7.4, 1 mM EDTA), the concentration of the DNA is assessed by spectrophotometry.

When the goal is to express a P1 clone comprising CanIon nucleotide sequences in a transgenic animal, typically in transgenic mice, it is desirable to remove vector sequences from the P1 DNA fragment, for example by cleaving the P1 DNA at rare-cutting sites within the P1 polylinker (SfiI, NotI or SalI). The P1 insert is then purified from vector sequences on a pulsed-field agarose gel, using methods similar using methods similar to those originally reported for the isolation of DNA from YACs (Schedl et al., 1993a; Peterson et al., 1993). At this stage, the resulting purified insert DNA can be concentrated, if necessary, on a Millipore Ultrafree-MC Filter Unit (Millipore, Bedford, Mass., USA—30,000 molecular weight limit) and then dialyzed against microinjection buffer (10 mM Tris-HCl, pH 7.4; 250 μM EDTA) containing 100 mM NaCl, 30 μM spermine, 70 μM spermidine on a microdyalisis membrane (type VS, 0.025 μM from Millipore). The intactness of the purified P1 DNA insert is assessed by electrophoresis on 1% agarose (Sea Kem GTG; FMC Bio-products) pulse-field gel and staining with ethidium bromide.

Baculovirus Vectors

A suitable vector for the expression of the CanIon polypeptide of SEQ ID No 5 or fragments or variants thereof is a baculovirus vector that can be propagated in insect cells and in insect cell lines. A specific suitable host vector system is the pVL1392/1393 baculovirus transfer vector (Pharmingen) that is used to transfect the SF9 cell line (ATCC No CRL 1711) which is derived from Spodoptera frugiperda.

Other suitable vectors for the expression of the CanIon polypeptide of SEQ ID No 5 or fragments or variants thereof in a baculovirus expression system include those described by Chai et al. (1993), Vlasak et al. (1983) and Lenhard et al. (1996).

Viral Vectors

In one specific embodiment, the vector is derived from an adenovirus. Preferred adenovirus vectors according to the invention are those described by Feldman and Steg (1996) or Ohno et al. (1994). Another preferred recombinant adenovirus according to this specific embodiment of the present invention is the human adenovirus type 2 or 5 (Ad 2 or Ad 5) or an adenovirus of animal origin (see, e.g., French patent application No FR-93.05954).

Retrovirus vectors and adeno-associated virus vectors are generally understood to be the recombinant gene delivery systems of choice for the transfer of exogenous polynucleotides in vivo, particularly to mammals, including humans. These vectors provide efficient delivery of genes into cells, and the transferred nucleic acids are stably integrated into the chromosomal DNA of the host.

Particularly preferred retroviruses for the preparation or construction of retroviral in vitro or in vitro gene delivery vehicles of the present invention include retroviruses selected from the group consisting of Mink-Cell Focus Inducing Virus, Murine Sarcoma Virus, Reticuloendotheliosis virus and Rous Sarcoma virus. Particularly preferred Murine Leukemia Viruses include the 4070A and the 1504A viruses, Abelson (ATCC No VR-999), Friend (ATCC No VR-245), Gross (ATCC No VR-590), Rauscher (ATCC No VR-998) and Moloney Murine Leukemia Virus (ATCC No VR-190; PCT Application No WO 94/24298). Particularly preferred Rous Sarcoma Viruses include Bryan high titer (ATCC Nos VR-334, VR-657, VR-726, VR-659 and VR-728). Other preferred retroviral vectors are those described in Roth et al. (1996), PCT Application No WO 93/25234, PCT Application No WO 94/06920, Roux et al., 1989, Julan et al., 1992 and Neda et al., 1991.

Yet another viral vector system that is contemplated by the invention comprises the adeno-associated virus (AAV). The adeno-associated virus is a naturally occurring defective virus that requires another virus, such as an adenovirus or a herpes virus, as a helper virus for efficient replication and a productive life cycle (Muzyczka et al., 1992). It is also one of the few viruses that may integrate its DNA into non-dividing cells, and exhibits a high frequency of stable integration (Flotte et al., 1992; Samulski et al., 1989; McLaughlin et al., 1989). One advantageous feature of AAV derives from its reduced efficacy for transducing primary cells relative to transformed cells.

BAC Vectors

The bacterial artificial chromosome (BAC) cloning system (Shizuya et al., 1992) has been developed to stably maintain large fragments of genomic DNA (100300 kb) in E. coli. A preferred BAC vector comprises a pBeloBAC11 vector that has been described by Kim et al. (1996). BAC libraries are prepared with this vector using size-selected genomic DNA that has been partially digested using enzymes that permit ligation into either the Bam HI or HindIII sites in the vector. Flanking these cloning sites are T7 and SP6 RNA polymerase transcription initiation sites that can be used to generate end probes by either RNA transcription or PCR methods. After the construction of a BAC library in E. coli, BAC DNA is purified from the host cell as a supercoiled circle. Converting these circular molecules into a linear form precedes both size determination and introduction of the BACs into recipient cells. The cloning site is flanked by two Not I sites, permitting cloned segments to be excised from the vector by NotI digestion. Alternatively, the DNA insert contained in the pBeloBAC11 vector may be linearized by treatment of the BAC vector with the commercially available enzyme lambda terminase that leads to the cleavage at the unique cosN site, but this cleavage method results in a full length BAC clone containing both the insert DNA and the BAC sequences.

5. Delivery of the Recombinant Vectors

In order to effect expression of the polynucleotides and polynucleotide constructs of the invention, these constructs must be delivered into a cell. This delivery may be accomplished in vitro, as in laboratory procedures for transforming cell lines, or in vivo or ex vivo, as in the treatment of certain diseases states.

One mechanism is viral infection where the expression construct is encapsulated in an infectious viral particle.

Several non-viral methods for the transfer of polynucleotides into cultured mammalian cells are also contemplated by the present invention, and include, without being limited to, calcium phosphate precipitation (Graham et al., 1973; Chen et al., 1987), DEAE-dextran (Gopal, 1985), electroporation (Tur-Kaspa et al., 1986; Potter et al., 1984), direct microinjection (Harland et al., 1985), DNA-loaded liposomes (Nicolau et al., 1982; Fraley et al., 1979), and receptor-mediated transfection (Wu and Wu, 1987; 1988). Some of these techniques may be successfully adapted for in vivo or ex vivo use.

Once the expression polynucleotide has been delivered into the cell, it may be stably integrated into the genome of the recipient cell. This integration may be in the cognate location and orientation via homologous recombination (gene replacement) or it may be integrated in a random, non specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle.

One specific embodiment for a method for delivering a protein or peptide to the interior of a cell of a vertebrate in vivo comprises the step of introducing a preparation comprising a physiologically acceptable carrier and a naked polynucleotide operatively coding for the polypeptide of interest into the interstitial space of a tissue comprising the cell, whereby the naked polynucleotide is taken up into the interior of the cell and has a physiological effect. This is particularly applicable for transfer in vitro but it may be applied to in vivo as well.

Compositions for use in vitro and in vivo comprising a “naked” polynucleotide are described in PCT application No. WO 90/11092 (Vical Inc.) and also in PCT application No. WO 95/11307 (Institut Pasteur, INSERM, Universite d'Ottawa) as well as in the articles of Tacson et al. (1996) and of Huygen et al. (1996).

In still another embodiment of the invention, the transfer of a naked polynucleotide of the invention, including a polynucleotide construct of the invention, into cells may be proceeded with a particle bombardment (biolistic), said particles being DNA-coated microprojectiles accelerated to a high velocity allowing them to pierce cell membranes and enter cells without killing them, such as described by Klein et al. (1987).

In a further embodiment, the polynucleotide of the invention may be entrapped in a liposome (Ghosh and Bacchawat, 1991; Wong et al., 1980; Nicolau et al., 1987)

In a specific embodiment, the invention provides a composition for the in vivo production of the CanIon protein or polypeptide described herein. It comprises a naked polynucleotide operatively coding for this polypeptide, in solution in a physiologically acceptable carrier, and suitable for introduction into a tissue to cause cells of the tissue to express the said protein or polypeptide.

The amount of vector to be injected to the desired host organism varies according to the site of injection. As an indicative dose, it will be injected between 0.1 and 100 μg of the vector in an animal body, preferably a mammalian body, for example a mouse body.

In another embodiment of the vector according to the invention, it may be introduced in vitro in a host cell, preferably in a host cell previously harvested from the animal to be treated and more preferably a somatic cell such as a muscle cell. In a subsequent step, the cell that has been transformed with the vector coding for the desired CanIon polypeptide or the desired fragment thereof is reintroduced into the animal body in order to deliver the recombinant protein within the body either locally or systemically.

### Cell Hosts

Another object of the invention comprises a host cell that has been transformed or transfected with one of the polynucleotides described herein, and in particular a polynucleotide either comprising a CanIon regulatory polynucleotide or the coding sequence of the CanIon polypeptide selected from the group consisting of SEQ ID Nos 1 to 4 or a fragment or a variant thereof. Also included are host cells that are transformed (prokaryotic cells) or that are transfected (eukaryotic cells) with a recombinant vector such as one of those described above. More particularly, the cell hosts of the present invention can comprise any of the polynucleotides described in the “Genomic Sequences Of The CanIon Gene” section, the “CanIon cDNA Sequences” section, the “Coding Regions” section, the “Polynucleotide constructs” section, and the “Oligonucleotide Probes And Primers” section.

A further recombinant cell host according to the invention comprises a polynucleotide containing a biallelic marker selected from the group consisting of A1 to A18, and the complements thereof.

An additional recombinant cell host according to the invention comprises any of the vectors described herein, more particularly any of the vectors described in the “Recombinant Vectors” section.

Preferred host cells used as recipients for the expression vectors of the invention are the following:

a) Prokaryotic host cells: Escherichia coli strains (I.E.DH5-α strain), Bacillus subtilis, Salmonella typhimurium, and strains from species like Pseudomonas, Streptomyces and Staphylococcus.

b) Eukaryotic host cells: HeLa cells (ATCC No CCL2; No CCL2.1; No CCL2.2), Cv 1 cells (ATCC No CCL70), COS cells (ATCC No CRL1650; No CRL1651), Sf-9 cells (ATCC No CRL1711), C127 cells (ATCC No CRL-1804), 3T3 (ATCC No CRL-6361), CHO (ATCC No CCL-61), human kidney 293. (ATCC No 45504; No CRL-1573) and BHK (ECACC No 84100501; No 84111301).

c) Other Mammalian Host Cells.

The CanIon gene expression in mammalian, and typically human, cells may be rendered defective, or alternatively it may be preceded with the insertion of a CanIon genomic or cDNA sequence with the replacement of the CanIon gene counterpart in the genome of an animal cell by a CanIon polynucleotide according to the invention. These genetic alterations may be generated by homologous recombination events using specific DNA constructs that have been previously described.

One kind of cell hosts that may be used are mammal zygotes, such as murine zygotes. For example, murine zygotes may undergo microinjection with a purified DNA molecule of interest, for example a purified DNA molecule that has previously been adjusted to a concentration range from 1 ng/ml—for BAC inserts—3 ng/ul—for P1 bacteriophage inserts—in 10 mM Tris-HCl, pH 7.4, 250 μM EDTA containing 100 mM NaCl, 30 μM spermine, and 70 μM spermidine. When the DNA to be microinjected has a large size, polyamines and high salt concentrations can be used in order to avoid mechanical breakage of this DNA, as described by Schedl et al. (1993b).

Any one of the polynucleotides of the invention, including the DNA constructs described herein, may be introduced in an embryonic stem (ES) cell line, preferably a mouse ES cell line. ES cell lines are derived from pluripotent, uncommitted cells of the inner cell mass of pre-implantation blastocysts. Preferred ES cell lines are the following: ES-E14TG2a (ATCC no CRL-1821), ES-D3 (ATCC no CRL1934 and no CRL-11632), YS001 (ATCC no CRL-11776), 36.5 (ATCC no CRL-11116). To maintain ES cells in an uncommitted state, they are cultured in the presence of growth inhibited feeder cells which provide the appropriate signals to preserve this embryonic phenotype and serve as a matrix for ES cell adherence. Preferred feeder cells are primary embryonic fibroblasts that are established from tissue of day 13-day 14 embryos of virtually any mouse strain, that are maintained in culture, such as described by Abbondanzo et al. (1993) and are inhibited in growth by irradiation, such as described by Robertson (1987), or by the presence of an inhibitory concentration of LIF, such as described by Pease and Williams (1990).

The constructs in the host cells can be used in a conventional manner to produce the gene product encoded by the recombinant sequence.

Following transformation of a suitable host and growth of the host to an appropriate cell density, the selected promoter is induced by appropriate means, such as temperature shift or chemical induction, and cells are cultivated for an additional period.

Cells are typically harvested by centrifugation, disrupted by physical or chemical means, and the resulting crude extract retained for further purification.

Microbial cells employed in the expression of proteins can be disrupted by any convenient method, including freeze-thaw cycling, sonication, mechanical disruption, or use of cell lysing agents. Such methods are well known by the skill artisan.

### Transgenic Animals

The terms “transgenic animals” or “host animals” are used herein to designate animals that have their genome genetically and artificially manipulated so as to include one of the nucleic acids according to the invention. Preferred animals are non-human mammals and include those belonging to a genus selected from Mus (e.g. mice), Rattus (e.g. rats) and Oryctogalus (e.g. rabbits) which have their genome artificially and genetically altered by the insertion of a nucleic acid according to the invention. In one embodiment, the invention encompasses non-human host mammals and animals comprising a recombinant vector of the invention or a CanIon gene disrupted by homologous recombination with a knock out vector.

The transgenic animals of the invention all include within a plurality of their cells a cloned recombinant or synthetic DNA sequence, more specifically one of the purified or isolated nucleic acids comprising a CanIon coding sequence, a CanIon regulatory polynucleotide, a polynucleotide construct, or a DNA sequence encoding an antisense polynucleotide such as described in the present specification.

Generally, a transgenic animal according the present invention comprises any one of the polynucleotides, the recombinant vectors and the cell hosts described in the present invention. More particularly, the transgenic animals of the present invention can comprise any of the polynucleotides described in the “Genomic Sequences Of The CanIon Gene” section, the “CanIon cDNA Sequences” section, the “Coding Regions” section, the “Polynucleotide constructs” section, the “Oligonucleotide Probes And Primers” section, the “Recombinant Vectors” section and the “Cell Hosts” section.

Further transgenic animals according to the invention contain in their somatic cells and/or in their germ line cells a polynucleotide comprising a biallelic marker selected from the group consisting of A1 to A18, and the complements thereof.

In a first preferred embodiment, these transgenic animals may be good experimental models in order to study the diverse pathologies related to cell differentiation, in particular concerning the transgenic animals within the genome of which has been inserted one or several copies of a polynucleotide encoding a native CanIon protein, or alternatively a mutant CanIon protein.

In a second preferred embodiment, these transgenic animals may express a desired polypeptide of interest under the control of the regulatory polynucleotides of the CanIon gene, leading to good yields in the synthesis of this protein of interest, and eventually a tissue specific expression of this protein of interest.

The design of the transgenic animals of the invention may be made according to the conventional techniques well known to those skilled in the art. Additional details regarding the production of transgenic animals, and specifically transgenic mice, can be found, e.g., in U.S. Pat. Nos. 4,873,191; 5,464,764; and 5,789,215, each of which is herein incorporated by reference.

Transgenic animals of the present invention are produced by the application of procedures which result in an animal with a genome that has incorporated exogenous genetic material. The procedure involves obtaining the genetic material, or a portion thereof, which encodes either a CanIon coding sequence, a CanIon regulatory polynucleotide or a DNA sequence encoding a CanIon antisense polynucleotide such as described in the present specification.

A recombinant polynucleotide of the invention is inserted into an embryonic or ES stem cell line. The insertion is preferably made using electroporation, such as described by Thomas et al. (1987). The cells subjected to electroporation are screened (e.g. by selection via selectable markers, by PCR or by Southern blot analysis) to find positive cells which have integrated the exogenous recombinant polynucleotide into their genome, preferably via an homologous recombination event. An illustrative positive-negative selection procedure that may be used according to the invention is described by Mansour et al. (1988).

Then, the positive cells are isolated, cloned and injected into 3.5 days old blastocysts from mice, such as described by Bradley (1987). The blastocysts are then inserted into a female host animal and allowed to grow to term.

Alternatively, the positive ES cells are brought into contact with embryos at the 2.5 days old 8-16 cell stage (morulae) such as described by Wood et al. (1993) or by Nagy et al. (1993), the ES cells being internalized to colonize extensively the blastocyst including the cells which will give rise to the germ line.

The offspring of the female host are tested to determine which animals are transgenic e.g. include the inserted exogenous DNA sequence and which are wild-type.

Thus, the present invention also concerns a transgenic animal containing a nucleic acid, a recombinant expression vector or a recombinant host cell according to the invention.

Recombinant Cell Lines Derived from the Transgenic Animals of the Invention.

A further object of the invention comprises recombinant host cells obtained from a transgenic animal described herein. In one embodiment the invention encompasses cells derived from non-human host mammals and animals comprising a recombinant vector of the invention or a CanIon gene disrupted by homologous recombination with a knock out vector.

Recombinant cell lines may be established in vitro from cells obtained from any tissue of a transgenic animal according to the invention, for example by transfection of primary cell cultures with vectors expressing one-genes such as SV40 large T antigen, as described by Chou (1989) and Shay et al. (1991).

### Methods of Screening for CanIon Modulators and Interacting Compounds

In numerous embodiments, the present invention provides compounds that interact with, bind to, or activate or inhibit the expression or activity of CanIon polypeptides, channels, and polynucleotides. Such compounds may be any organic or inorganic compound, including, but not limited to, polypeptides, polynucleotides, lipids, carbohydrates, nucleotides, amino acids, or small molecule inhibitors or activators. As described elsewhere in this application, such compounds are useful for the treatment or prevention of any of a large number of diseases or conditions. Preferably, inhibitors of CanIon activity or expression are used in the treatment or prevention of a psychiatric disorder such as schizophrenia or bipolar disorder.

### Methods of Screening for CanIon Channel Modulators

Compounds capable of binding to CanIon and compounds capable of modulating CanIon function have important applications in the treatment of disease. Voltage-gated ion channels are generally well established as drug targets because they are pharmacologically accessible, encoded by a variety of genes and usually operate as multimeric protein assemblies, resulting in a high degree of functional and anatomical specificity. Furthermore, because ion channel opening and closing involving the movement of charged voltage sensitive amino acids leads to changes in conformation states, ion channels allow the design of state dependent molecules that, for example, bind only to channels that are in conducting (activated) or non-conducting (inactivated) state.

In addition, numerous calcium channel modulators have been demonstrated to be efficacious in the treatment or prevention of numerous diseases and conditions. For example, calcium channel inhibitors have been shown to be effective against various cardiovascular diseases and conditions (e.g., angina, arrythmias, hypertension), as well as CNS and neuronal disorders (e.g., migraines, neurological effects of strokes, mania, neuroleptic-induced tardive dyskinesia, schizophrenia, bipolar disorder, pain, epilepsy, and others). In addition, calcium channel agonists have been shown to be effective for various applications, such as in reducing the duration of and otherwise attenuating the effects of local anesthesia. Antagonists and agonists of CanIon channels are similarly useful in the treatment or prevention of these and other diseases and conditions. For example, CanIon antagonists are useful in the treatment or prevention of schizophrenia and bipolar disorder.

Because voltage gated ion channels do not require agonist binding for activation, compounds are preferably screened against functional CanIon channels. Assays may include functional and radioligand binding approaches applied to cells (vesicles or membranes) expressing native or cloned channels, or to whole cell assays. Functional whole cell assays may use electrophysiological techniques, such as patch clamping. Assays may involve any voltage-gated channel type, preferably L, N, and T type channels. Kinetic ion flux through the channel may also be measured, e.g., using fluorescence, end-point radiotracer or cell viability techniques.

Assays may also make use of various toxins, venoms or compounds that bind to and open or close channels (Denyer et al., Drug Disc. Today 3(7): 323-332 (1998), incorporated herein by reference in its entirety). In one embodiment, the present assays involve the use of any of the large number of known calcium channel agonists and antagonists, e.g., as positive or negative controls. Examples of suitable known calcium channel antagonists include phenylalkylamines (e.g., verapamil), benzothiazepines (e.g., diltiazem), and dihydropyridines (e.g., nifedipine); calcium channel agonists include FPL-64176 and BAY K 8644; sodium channel agonists include Batrachotoxin; and sodium channel antagonists include spiradoline, mexiletine, U-54494A ((+/−)-cis-3,4-Dichloro-N-methyl-N-[2-(1-pyrrolidinyl)-cyclohexyl]-benzamide). Such compounds may also be used as “lead” compounds, i.e. to serve as starting molecules for the design or discovery of derivative molecules that specifically bind to or modulate CanIon channels.

In preferred embodiments, assays of the invention comprise a method for the screening of a candidate substance comprises the following steps:

a) providing (i) a sample or a host cell containing a polypeptide comprising, consisting essentially of, or consisting of a CanIon protein or a fragment thereof, or (ii) a recombinant host cell expressing a polynucleotide encoding a polypeptide comprising, consisting essentially of, or consisting of a CanIon protein or a fragment thereof;

b) obtaining a candidate substance;

c) bringing into contact said host cell with said candidate substance;

d) determining the effect of said candidate substance on CanIon activity.

Determining the effect of the candidate substance on CanIon activity can be accomplished according to well known methods. Preferably, the effect of the candidate substance on CanIon activity is an agonist or an antagonist effect. Generally, a compound inhibits CanIon if the ability to transport ions (eg. Ca2+ or Na+) is decreased. A compound stimulates CanIon if the ability to transportions is increased.

CanIon activity can be detected using any suitable means. In preferred examples, CanIon activity is detected by measuring a signaling event. While a signaling event may comprise any suitable change of a molecular characteristic or parameter of the cell, nonlimiting examples of a signaling event include changes in ion fluxes, such as changes in or generation of a Ca2+ or Na+, or K+ flux or enzyme activation.

In one aspect, ion flux can be monitored by measuring electrophysiological properties of the CanIon channel, using for example techniques for measuring whole cell current from a single cell or in membrane patches. In other examples, fluorescent or radioactive labels can be used to detect displacement of a known CanIon-binding compound, or to detect ion flux in a across a cell (eg labelled Ca2+ or Na+). An indicator for the physiological parameters of a cell can be used, such as a fluorescent indicator for cell viability. In other examples, change in the physical location of an indicator can be detected, such as the use of fluorescence activated cell sorting to identify exclusion or uptake of a physiological indicator.

The sample used in the assay of the invention contains a polypeptide or a host cell expressing a polypeptide comprising, consisting essentially of, or consisting of a CanIon protein or a fragment thereof, or (ii) a recombinant host cell expressing a polynucleotide encoding a polypeptide comprising, consisting essentially of, or consisting of a CanIon protein or a fragment thereof. Preferably, CanIon assays of the invention involve the use of a recombinant host cell expressing a functional CanIon polypeptide. Host cells may express or comprise a functional alpha subunit of CanIon channel, or may express or comprise one or more additional ion channel subunits, or a ion channel complex comprising CanIon. Preferably, a host cell is used which has low endogenous ion channel expression or have low background ion, particularly Ca2+ and/or Na+, conductance.

Radioligand Binding

In one aspect, a CanIon channel may be screened by identifying a high affinity ligand that binds to a site of interest of CanIon and preferably has a desired modulatory effect, and detecting the ability of a test compound to displace said labelled ligand. Lists of toxicological/pharmacological agents used in voltage gated (Ca2+, Na+ and K+) channels assays are provided in Denyer et al. (supra). This method is generally suitable for detecting compounds which bind to the same site, or are allosterically coupled to the site, as the labelled ligand, but does not provide information as to agonist or antagonist properties of the compound.

Cell Based Fluorescence and Radiotracer Assays

In another assay, CanIon function can be monitored by measuring changes in intracellular concentration of permeant ion using fluorescent-ion indicators or radiolabelled ions.

Typically, ion channels such as Na+ channels inactivate in milliseconds after voltage stimulation. Ca2+ channels exhibit no or a lesser degree of inactivation and can be opened by high K+ depolarization. In cell based fluorescence and radiotracer assays, the CanIon channel can be generally activated by a toxin or any test compound, or high K+ depolarization, such that the channel is opened for prolonged periods (up to many minutes).

In fluorescence based assays, fluorescent C2+ dyes are available for use (e.g., Fluo-3, Calcium green-1, Molecular Probes, OR, U.S.A). Ca2+ channels can be activated by depolarizing the membrane with an isotonic solution or Na+ channels with a toxin or other compound, and the resulting transient movement of fluoresence in the cell can be measured over 20 to 60s. Fluorescence measurement systems and devices are further described in Denyer et al. (supra). Radiotracers 22Na+ and 14C-guanidine are commonly used for Na+ channel analysis and 45Ca2+ for Ca2+ channel analysis. In a preferred embodiment described in Denyer et al. (supra), Cytostar-T scintillating microplates (Amersham International, U.K.) are used to perform high throughput CanIon cell based assays.

In further assays, Ca2+ function of an ion channel is monitored by measuring membrane potential with a membrane potential indicator, High electrical resistance of biological membranes allow small ionic currents across the plasma membrane to cause large changes in membrane potential. Voltage assays can thus be conveniently used to detect generic ion flux across membranes. Cell lines are generally chosen so that effects from endogenous ion channels are minimized. A range of dyes are available as membrane potential indicator dyes, divided into fast and slow response dyes, as well as FRET-based voltage sensor dyes. (Aurora Biosciences, CA, USA; reviewed in Gonzalez et al., Drug Disc. Today 4(9): 431:439 (1999)

Cell Viability

In cell viability assays, ion channel activity and ion flux are directly related to cell viability. Both yeast and mammalian cell systems are available for testing an ion channel target. For example, a yeast system employing an ion-specific K+ uptake deficient Saccharomyces cerevisiae cell line has be used, in which a functional K+ channel of interest is expressed in the cell line thereby restoring K+ uptake and promoting cell survival. (Anderson et al., Symp. Soc. Exp. Biol. 48: 85-97 (1994)) Such an assay for Ca2+ or Na+ channels may be used to identify compounds capable of blocking CanIon function. Mammalian cell systems are also available, such as a Na+ channel assay using mammalian neuroblastoma cells with a calorimetric cell viability readout. Cells treated with a Na+ channel opener and a Na+/K+ pump inhibitor to promote a lethal intracellular Na+ overload. Treatment with a test compound capable of blocking the channel will improve cell viability, which compounds which enhance channel opening will further promote cell death. (Manger et al., Anal. Biochem. 214: 190-194 (1993).

Electrophysiology

Electrophysiological voltage-clamping techniques involve the measurement of ionic current flowing through one or many channels. A single microelectrode to control the membrane voltage while the current flow is measured through a single cell or membrane patch. (Hamill, Pfugers Arch. 391, 85-100 (1981). Ionic current can thus be measured in the presence or absence of a test compound of interest. A large scale compound screening system has been designed (Neurosearch A/S, Glostrup, Denmark; Olesen et al., Voltage gated ion channel modulators, 7-8 December, Philadelphia Pa., USA (1995); Denyer et al., supra).

Methods for Screening for Substances Interacting with a CanIon Polypeptide

For the purpose of the present invention, a ligand means a molecule, such as a protein, a peptide, an antibody or any synthetic chemical compound capable of binding to the CanIon protein or one of its fragments or variants or to modulate the expression of the polynucleotide coding for CanIon or a fragment or variant thereof.

In the ligand screening method according to the present invention, a biological sample or a defined molecule to be tested as a putative ligand of the CanIon protein is brought into contact with the corresponding purified CanIon protein, for example the corresponding purified recombinant CanIon protein produced by a recombinant cell host as described hereinbefore, in order to form a complex between this protein and the putative ligand molecule to be tested.

As an illustrative example, to study the interaction of the CanIon protein, or a fragment comprising a contiguous span of at least 6 amino acids, preferably at least 8 to 10 amino acids, more preferably at least 12, 15, 20, 25, 30, 40, 50, 100, 200, 300, 400, 500 or 1000 amino acids of SEQ ID No 5, with drugs or small molecules, such as molecules generated through combinatorial chemistry approaches, the microdialysis coupled to HPLC method described by Wang et al. (1997) or the affinity capillary electrophoresis method described by Bush et al. (1997), the disclosures of which are incorporated by reference, can be used.

In further methods, peptides, drugs, fatty acids, lipoproteins, or small molecules which interact with the CanIon protein, or a fragment comprising a contiguous span of at least 6 amino acids, preferably at least 8 to 10 amino acids, more preferably at least 12, 15, 20, 25, 30, 40, 50, or 100 amino acids of SEQ ID No 5, may be identified using assays such as the following. The molecule to be tested for binding is labeled with a detectable label, such as a fluorescent, radioactive, or enzymatic tag and placed in contact with immobilized CanIon protein, or a fragment thereof under conditions which permit specific binding to occur. After removal of non-specifically bound molecules, bound molecules are detected using appropriate means.

Another object of the present invention comprises methods and kits for the screening of candidate substances that interact with CanIon polypeptide.

The present invention pertains to methods for screening substances of interest that interact with a CanIon protein or one fragment or variant thereof. By their capacity to bind covalently or non-covalently to a CanIon protein or to a fragment or variant thereof, these substances or molecules may be advantageously used both in vitro and in vivo.

In vitro, said interacting molecules may be used as detection means in order to identify the presence of a CanIon protein in a sample, preferably a biological sample.

A method for the screening of a candidate substance comprises the following steps

a) providing a polypeptide comprising, consisting essentially of, or consisting of a CanIon protein or a fragment comprising a contiguous span of at least 6 amino acids, preferably at least 8 to 10 amino acids, more preferably at least 12, 15, 20, 25, 30, 40, 50, or 100 amino acids of SEQ ID No 5;

b) obtaining a candidate substance;

c) bringing into contact said polypeptide with said candidate substance;

d) detecting the complexes formed between said polypeptide and said candidate substance.

The invention further concerns a kit for the screening of a candidate substance interacting with the CanIon polypeptide, wherein said kit comprises:

a) a CanIon protein having an amino acid sequence selected from the group consisting of the amino acid sequences of SEQ ID No 5 or a peptide fragment comprising a contiguous span of at least 6 amino acids, preferably at least 8 to 10 amino acids, more preferably at least 12, 15, 20, 25, 30, 40, 50, or 100 amino acids of SEQ ID No 5;

b) optionally means useful to detect the complex formed between the CanIon protein or a peptide fragment or a variant thereof and the candidate substance.

In a preferred embodiment of the kit described above, the detection means comprises a monoclonal or polyclonal antibodies directed against the CanIon protein or a peptide fragment or a variant thereof.

Various candidate substances or molecules can be assayed for interaction with a CanIon polypeptide. These substances or molecules include, without being limited to, natural or synthetic organic compounds or molecules of biological origin such as polypeptides. When the candidate substance or molecule comprises a polypeptide, this polypeptide may be the resulting expression product of a phage clone belonging to a phage-based random peptide library, or alternatively the polypeptide may be the resulting expression product of a cDNA library cloned in a vector suitable for performing a two-hybrid screening assay.

The invention also pertains to kits useful for performing the hereinbefore described screening method. Preferably, such kits comprise a CanIon polypeptide or a fragment or a variant thereof, and optionally means useful to detect the complex formed between the CanIon polypeptide or its fragment or variant and the candidate substance. In a preferred embodiment the detection means comprise a monoclonal or polyclonal antibodies directed against the corresponding CanIon polypeptide or a fragment or a variant thereof.

A. Candidate Ligands Obtained from Random Peptide Libraries

In a particular embodiment of the screening method, the putative ligand is the expression product of a DNA insert contained in a phage vector (Parmley and Smith, 1988). Specifically, random peptide phage libraries are used. The random DNA inserts encode for peptides of 8 to 20 amino acids in length (Oldenburg K. R. et al., 1992; Valadon P., et al., 1996; Lucas A. H., 1994; Westerink M. A. J., 1995; Felici F. et al., 1991). According to this particular embodiment, the recombinant phages expressing a protein that binds to the immobilized CanIon protein is retained and the complex formed between the CanIon protein and the recombinant phage may be subsequently immunoprecipitated by a polyclonal or a monoclonal antibody directed against the CanIon protein.

Once the ligand library in recombinant phages has been constructed, the phage population is brought into contact with the immobilized CanIon protein. Then the preparation of complexes is washed in order to remove the non-specifically bound recombinant phages. The phages that bind specifically to the CanIon protein are then eluted by a buffer (acid pH) or immunoprecipitated by the monoclonal antibody produced by the hybridoma anti-CanIon, and this phage population is subsequently amplified by an over-infection of bacteria (for example E. coli). The selection step may be repeated several times, preferably 2-4 times, in order to select the more specific recombinant phage clones. The last step comprises characterizing the peptide produced by the selected recombinant phage clones either by expression in infected bacteria and isolation, expressing the phage insert in another host-vector system, or sequencing the insert contained in the selected recombinant phages.

B. Candidate Ligands Obtained by Competition Experiments.

Alternatively, peptides, drugs or small molecules which bind to the CanIon protein, or a fragment comprising a contiguous span of at least 6 amino acids, preferably at least 8 to 10 amino acids, more preferably at least 12, 15, 20, 25, 30, 40, 50, or 100 amino acids of SEQ ID No 5, may be identified in competition experiments. In such assays, the CanIon protein, or a fragment thereof, is immobilized to a surface, such as a plastic plate. Increasing amounts of the peptides, drugs or small molecules are placed in contact with the immobilized CanIon protein, or a fragment thereof, in the presence of a detectable labeled known CanIon protein ligand. For example, the CanIon ligand may be detectably labeled with a fluorescent, radioactive, or enzymatic tag. The ability of the test molecule to bind the CanIon protein, or a fragment thereof, is determined by measuring the amount of detectably labeled known ligand bound in the presence of the test molecule. A decrease in the amount of known ligand bound to the CanIon protein, or a fragment thereof, when the test molecule is present indicated that the test molecule is able to bind to the CanIon protein, or a fragment thereof.

C. Candidate Ligands Obtained by Affinity Chromatography.

Proteins or other molecules interacting with the CanIon protein, or a fragment comprising a contiguous span of at least 6 amino acids, preferably at least 8 to 10 amino acids, more preferably at least 12, 15, 20, 25, 30, 40, 50, or 100 amino acids of SEQ ID No 5. The CanIon protein, or a fragment thereof, may be attached to the column using conventional techniques including chemical coupling to a suitable column matrix such as agarose, Affi Gel®, or other matrices familiar to those of skill in art. In some embodiments of this method, the affinity column contains chimeric proteins in which the CanIon protein, or a fragment thereof, is fused to glutathion S transferase (GST). A mixture of cellular proteins or pool of expressed proteins as described above is applied to the affinity column. Proteins or other molecules interacting with the CanIon protein, or a fragment thereof, attached to the column can then be isolated and analyzed on 2-D electrophoresis gel as described in Ramunsen et al. (1997), the disclosure of which is incorporated by reference. Alternatively, the proteins retained on the affinity column can be purified by electrophoresis based methods and sequenced. The same method can be used to isolate antibodies, to screen phage display products, or to screen phage display human antibodies.

D. Candidate Ligands Obtained by Optical Biosensor Methods

Proteins interacting with the CanIon protein, or a fragment comprising a contiguous span of at least 6 amino acids, preferably at least 8 to 10 amino acids, more preferably at least 12, 15, 20, 25, 30, 40, 50, or 100 amino acids of SEQ ID No 5, can also be screened by using an Optical Biosensor as described in Edwards and Leatherbarrow (1997) and also in Szabo et al. (1995), the disclosure of which is incorporated by reference. This technique permits the detection of interactions between molecules in real time, without the need of labeled molecules. This technique is based on the surface plasmon resonance (SPR) phenomenon. Briefly, the candidate ligand molecule to be tested is attached to a surface (such as a carboxymethyl dextran matrix). A light beam is directed towards the side of the surface that does not contain the sample to be tested and is reflected by said surface. The SPR phenomenon causes a decrease in the intensity of the reflected light with a specific association of angle and wavelength. The binding of candidate ligand molecules cause a change in the refraction index on the surface, which change is detected as a change in the SPR signal. For screening of candidate ligand molecules or substances that are able to interact with the CanIon protein, or a fragment thereof, the CanIon protein, or a fragment thereof, is immobilized onto a surface. This surface comprises one side of a cell through which flows the candidate molecule to be assayed. The binding of the candidate molecule on the CanIon protein, or a fragment thereof, is detected as a change of the SPR signal. The candidate molecules tested may be proteins, peptides, carbohydrates, lipids, or small molecules generated by combinatorial chemistry. This technique may also be performed by immobilizing eukaryotic or prokaryotic cells or lipid vesicles exhibiting an endogenous or a recombinantly expressed CanIon protein at their surface.

The main advantage of the method is that it allows the determination of the association rate between the CanIon protein and molecules interacting with the CanIon protein. It is thus possible to select specifically ligand molecules interacting with the CanIon protein, or a fragment thereof, through strong or conversely weak association constants.

E. Candidate Ligands Obtained Through a Two-Hybrid Screening Assay.

The yeast two-hybrid system is designed to study protein-protein interactions in vivo (Fields and Song, 1989), and relies upon the fusion of a bait protein to the DNA binding domain of the yeast Gal4 protein. This technique is also described in the U.S. Pat. Nos. 5,667,973 and 5,283,173 (Fields et al.) the technical teachings of both patents being herein incorporated by reference.

The general procedure of library screening by the two-hybrid assay may be performed as described by Harper et al. (1993), Cho et al. (1998), or Fromont-Racine et al. (1997).

The bait protein or polypeptide comprises, consists essentially of, or consists of a CanIon polypeptide or a fragment comprising a contiguous span of at least 6 amino acids, preferably at least 8 to 10 amino acids, more preferably at least 12, 15, 20, 25, 30, 40, 50, or 100 amino acids of SEQ ID No 5.

More precisely, the nucleotide sequence encoding the CanIon polypeptide or a fragment or variant thereof is fused to a polynucleotide encoding the DNA binding domain of the GAL4 protein, the fused nucleotide sequence being inserted in a suitable expression vector, for example pAS2 or pM3.

Then, a human cDNA library is constructed in a specially designed vector, such that the human cDNA insert is fused to a nucleotide sequence in the vector that encodes the transcriptional domain of the GAL4 protein. Preferably, the vector used is the pACT vector. The polypeptides encoded by the nucleotide inserts of the human cDNA library are termed “prey” polypeptides.

A third vector contains a detectable marker gene, such as beta galactosidase gene or CAT gene that is placed under the control of a regulation sequence that is responsive to the binding of a complete Gal4 protein containing both the transcriptional activation domain and the DNA binding domain. For example, the vector pG5EC may be used.

Two different yeast strains are also used. As an illustrative but non limiting example the two different yeast strains may be the followings:
Y190, the phenotype of which is (MATa, Leu2-3, 112 ura3-12, trp1-901, his3-D200, ade2-101, gal4Dgal180D URA3 GAL-LacZ, LYS GAL-HIS3, cyh);
Y187, the phenotype of which is (MATa gal4 gal80 his3 trp1-901 ade2-101 ura3-52 leu2-3, -112 URA3 GAL-lacZmet⁻), which is the opposite mating type of Y190.

Briefly, 20 μg of pAS2/CanIon and 20 μg of pACT-cDNA library are co-transformed into yeast strain Y190. The transformants are selected for growth on minimal media lacking histidine, leucine and tryptophan, but containing the histidine synthesis inhibitor 3-AT (50 mM). Positive colonies are screened for beta galactosidase by filter lift assay. The double positive colonies (His⁻, beta-gal⁺) are then grown on plates lacking histidine, leucine, but containing tryptophan and cycloheximide (10 mg/ml) to select for loss of pAS2/CanIon plasmids bu retention of pACT-cDNA library plasmids. The resulting Y190 strains are mated with Y187 strains expressing CanIon or non-related control proteins; such as cyclophilin B, lamin, or SNF1, as Gal4 fusions as described by Harper et al. (1993) and by Bram et al. (Bram R J et al., 1993), and screened for beta galactosidase by filter lift assay. Yeast clones that are beta gal-after mating with the control Gal4 fusions are considered false positives.

In another embodiment of the two-hybrid method according to the invention, interaction between the CanIon or a fragment or variant thereof with cellular proteins may be assessed using the Matchmaker Two Hybrid System 2 (Catalog No. K1604-1, Clontech). As described in the manual accompanying the Matchmaker Two Hybrid System 2 (Catalog No. K1604-1, Clontech), the disclosure of which is incorporated herein by reference, nucleic acids encoding the CanIon protein or a portion thereof, are inserted into an expression vector such that they are in frame with DNA encoding the DNA binding domain of the yeast transcriptional activator GAL4. A desired cDNA, preferably human cDNA, is inserted into a second expression vector such that they are in frame with DNA encoding the activation domain of GAL4. The two expression plasmids are transformed into yeast and the yeast are plated on selection medium which selects for expression of selectable markers on each of the expression vectors as well as GAL4 dependent expression of the HIS3 gene. Transformants capable of growing on medium lacking histidine are screened for GAL4 dependent lacZ expression. Those cells which are positive in both the histidine selection and the lacZ assay contain interaction between CanIon and the protein or peptide encoded by the initially selected cDNA insert.

Method for Screening Substances Interacting with the Regulatory Sequences of the CanIon Gene.

The present invention also concerns a method for screening substances or molecules that are able to interact with the regulatory sequences of the CanIon gene, such as for example promoter or enhancer sequences.

Nucleic acids encoding proteins which are able to interact with the regulatory sequences of the CanIon gene, more particularly a nucleotide sequence selected from the group consisting of the polynucleotides of the 5′ and 3′ regulatory region or a fragment or variant thereof, and preferably a variant comprising one of the biallelic markers of the invention, may be identified by using a one-hybrid system, such as that described in the booklet enclosed in the Matchmaker One-Hybrid System kit from Clontech (Catalog Ref. no K1603-1), the technical teachings of which are herein incorporated by reference. Briefly, the target nucleotide sequence is cloned upstream of a selectable reporter sequence and the resulting DNA construct is integrated in the yeast genome (Saccharomyces cerevisiae). The yeast cells containing the reporter sequence in their genome are then transformed with a library comprising fusion molecules between cDNAs encoding candidate proteins for binding onto the regulatory sequences of the CanIon gene and sequences encoding the activator domain of a yeast transcription factor such as GAL4. The recombinant yeast cells are plated in a culture broth for selecting cells expressing the reporter sequence. The recombinant yeast cells thus selected contain a fusion protein that is able to bind onto the target regulatory sequence of the CanIon gene. Then, the cDNAs encoding the fusion proteins are sequenced and may be cloned into expression or transcription vectors in vitro. The binding of the encoded polypeptides to the target regulatory sequences of the CanIon gene may be confirmed by techniques familiar to the one skilled in the art, such as gel retardation assays or DNAse protection assays.

Gel retardation assays may also be performed independently in order to screen candidate molecules that are able to interact with the regulatory sequences of the CanIon gene, such as described by Fried and Crothers (1981), Garner and Revzin (1981) and Dent and Latchman (1993), the teachings of these publications being herein incorporated by reference. These techniques are based on the principle according to which a DNA fragment which is bound to a protein migrates slower than the same unbound DNA fragment. Briefly, the target nucleotide sequence is labeled. Then the labeled target nucleotide sequence is brought into contact with either a total nuclear extract from cells containing transcription factors, or with different candidate molecules to be tested. The interaction between the target regulatory sequence of the CanIon gene and the candidate molecule or the transcription factor is detected after gel or capillary electrophoresis through a retardation in the migration.

Method for Screening Ligands that Modulate the Expression of the CanIon Gene.

Another subject of the present invention is a method for screening molecules that modulate the expression of the CanIon protein. Such a screening method comprises the steps of:

a) cultivating a prokaryotic or an eukaryotic cell that has been transfected with a nucleotide sequence encoding the CanIon protein or a variant or a fragment thereof, placed under the control of its own promoter;

b) bringing into contact the cultivated cell with a molecule to be tested;

c) quantifying the expression of the CanIon protein or a variant or a fragment thereof.

In an embodiment, the nucleotide sequence encoding the CanIon protein or a variant or a fragment thereof comprises an allele of at least one of the biallelic markers A 12 or A16, and the complements thereof.

Using DNA recombination techniques well known by the one skill in the art, the CanIon protein encoding DNA sequence is inserted into an expression vector, downstream from its promoter sequence. As an illustrative example, the promoter sequence of the CanIon gene is contained in the nucleic acid of the 5′ regulatory region.

The quantification of the expression of the CanIon protein may be realized either at the mRNA level or at the protein level. In the latter case, polyclonal or monoclonal antibodies may be used to quantify the amounts of the CanIon protein that have been produced, for example in an ELISA or a RIA assay.

In a preferred embodiment, the quantification of the CanIon mRNA is realized by a quantitative PCR amplification of the cDNA obtained by a reverse transcription of the total mRNA of the cultivated CanIon-transfected host cell, using a pair of primers specific for CanIon.

The present invention also concerns a method for screening substances or molecules that are able to increase or decrease the level of expression of the CanIon gene. Such a method may allow one skilled in the art to select substances exerting a regulating effect on the expression level of the CanIon gene and which may be useful as active ingredients included in pharmaceutical compositions for treating patients suffering from any of the herein-described diseases.

Thus, the present invention also provides a method for screening of a candidate substance or molecule that modulated the expression of the CanIon gene, this method comprises the following steps:
providing a recombinant cell host containing a nucleic acid, wherein said nucleic acid comprises a nucleotide sequence of the 5′ regulatory region or a biologically active fragment or variant thereof located upstream a polynucleotide encoding a detectable protein;
obtaining a candidate substance; and
determining the ability of the candidate substance to modulate the expression levels of the polynucleotide encoding the detectable protein.

In a further embodiment, the nucleic acid comprising the nucleotide sequence of the 5′ regulatory region or a biologically active fragment or variant thereof also includes a 5′UTR region of the CanIon cDNA of SEQ ID No 4, or one of its biologically active fragments or variants thereof.

Among the preferred polynucleotides encoding a detectable protein, there may be cited polynucleotides encoding beta galactosidase, green fluorescent protein (GFP) and chloramphenicol acetyl transferase (CAT).

The invention also pertains to kits useful for performing the herein described screening method. Preferably, such kits comprise a recombinant vector that allows the expression of a nucleotide sequence of the 5′ regulatory region or a biologically active fragment or variant thereof located upstream and operably linked to a polynucleotide encoding a detectable protein or the CanIon protein or a fragment or a variant thereof.

In another method for the screening of a candidate substance or molecule that modulates the expression of the CanIon gene, the method comprises the following steps:

a) providing a recombinant host cell containing a nucleic acid, wherein said nucleic acid comprises a 5′UTR sequence of the CanIon cDNA of SEQ ID No 4, or one of its biologically active fragments or variants, the 5′UTR sequence or its biologically active fragment or variant being operably linked to a polynucleotide encoding a detectable protein;

b) obtaining a candidate substance; and

c) determining the ability of the candidate substance to modulate the expression levels of the polynucleotide encoding the detectable protein.

In a specific embodiment of the above screening method, the nucleic acid that comprises a nucleotide sequence selected from the group consisting of the 5′UTR sequence of the CanIon cDNA of SEQ ID No 4 or one of its biologically active fragments or variants, includes a promoter sequence which is endogenous with respect to the CanIon 5′UTR sequence.

In another specific embodiment of the above screening method, the nucleic acid that comprises a nucleotide sequence selected from the group consisting of the 5′UTR sequence of the CanIon cDNA of SEQ ID No 4 or one of its biologically active fragments or variants, includes a promoter sequence which is exogenous with respect to the CanIon 5′UTR sequence defined therein.

In a further preferred embodiment, the nucleic acid comprising the 5′-UTR sequence of the CanIon cDNA or SEQ ID No 4 or the biologically active fragments thereof includes a biallelic marker selected from the group consisting of A12 or A 16 or the complements thereof.

The invention further comprises a kit for the screening of a candidate substance modulating the expression of the CanIon gene, wherein said kit comprises a recombinant vector that comprises a nucleic acid including a 5′UTR sequence of the CanIon cDNA of SEQ ID No 4, or one of their biologically active fragments or variants, the 5′UTR sequence or its biologically active fragment or variant being operably linked to a polynucleotide encoding a detectable protein.

Expression levels and patterns of CanIon may be analyzed by solution hybridization with long probes as described in International Patent Application No. WO 97/05277, the entire contents of which are incorporated herein by reference. Briefly, the CanIon cDNA or the CanIon genomic DNA described above, or fragments thereof, is inserted at a cloning site immediately downstream of a bacteriophage (T3, T7 or SP6) RNA polymerase promoter to produce antisense RNA. Preferably, the CanIon insert comprises at least 100 or more consecutive nucleotides of the genomic DNA sequence or the cDNA sequences. The plasmid is linearized and transcribed in the presence of ribonucleotides comprising modified ribonucleotides (i.e. biotin-UTP and DIG-UTP). An excess of this doubly labeled RNA is hybridized in solution with mRNA isolated from cells or tissues of interest. The hybridization is performed under standard stringent conditions (40-50° C. for 16 hours in an 80% formamide, 0.4 M NaCl buffer, pH 7-8). The unhybridized probe is removed by digestion with ribonucleases specific for single-stranded RNA (i.e. RNases CL3, Tl, Phy M, U2 or A). The presence of the biotin-UTP modification enables capture of the hybrid on a microtitration plate coated with streptavidin. The presence of the DIG modification enables the hybrid to be detected and quantified by ELISA using an anti-DIG antibody coupled to alkaline phosphatase.

Quantitative analysis of CanIon gene expression may also be performed using arrays. As used herein, the term array means a one dimensional, two dimensional, or multidimensional arrangement of a plurality of nucleic acids of sufficient length to permit specific detection of expression of mRNAs capable of hybridizing thereto. For example, the arrays may contain a plurality of nucleic acids derived from genes whose expression levels are to be assessed. The arrays may include the CanIon genomic DNA, the CanIon cDNA sequences or the sequences complementary thereto or fragments thereof, particularly those comprising at least one of the biallelic markers according the present invention, preferably at least one of the biallelic markers A1 to A17. Preferably, the fragments are at least 15 nucleotides in length. In other embodiments, the fragments are at least 25 nucleotides in length. In some embodiments, the fragments are at least 50 nucleotides in length. More preferably, the fragments are at least 100 nucleotides in length. In another preferred embodiment, the fragments are more than 100 nucleotides in length. In some embodiments the fragments may be more than 500 nucleotides in length.

For example, quantitative analysis of CanIon gene expression may be performed with a complementary DNA microarray as described by Schena et al. (1995 and 1996). Full length CanIon cDNAs or fragments thereof are amplified by PCR and arrayed from a 96-well microtiter plate onto silylated microscope slides using high-speed robotics. Printed arrays are incubated in a humid chamber to allow rehydration of the array elements and rinsed, once in 0.2% SDS for 1 min, twice in water for 1 min and once for 5 min in sodium borohydride solution. The arrays are submerged in water for 2 min at 95° C., transferred into 0.2% SDS for 1 min, rinsed twice with water, air dried and stored in the dark at 25° C.

Cell or tissue mRNA is isolated or commercially obtained and probes are prepared by a single round of reverse transcription. Probes are hybridized to 1 cm² microarrays under a 14×14 mm glass coverslip for 6-12 hours at 60° C. Arrays are washed for 5 min at 25° C. in low stringency wash buffer (1×SSC/0.2% SDS), then for 10 min at room temperature in high stringency wash buffer (0.1×SSC/0.2% SDS). Arrays are scanned in 0.1×SSC using a fluorescence laser scanning device fitted with a custom filter set. Accurate differential expression measurements are obtained by taking the average of the ratios of two independent hybridizations.

Quantitative analysis of CanIon gene expression may also be performed with full length CanIon cDNAs or fragments thereof in complementary DNA arrays as described by Pietu et al. (1996). The full length CanIon cDNA or fragments thereof is PCR amplified and spotted on membranes. Then, mRNAs originating from various tissues or cells are labeled with radioactive nucleotides. After hybridization and washing in controlled conditions, the hybridized mRNAs are detected by phospho-imaging or autoradiography. Duplicate experiments are performed and a quantitative analysis of differentially expressed mRNAs is then performed.

Alternatively, expression analysis using the CanIon genomic DNA, the CanIon cDNA, or fragments thereof can be done through high density nucleotide arrays as described by Lockhart et al. (1996) and Sosnowsky et al. (1997). Oligonucleotides of 15-50 nucleotides from the sequences of the CanIon genomic DNA or the CanIon cDNA sequences, particularly those comprising at least one of biallelic markers according the present invention, preferably at least one biallelic marker selected from the group consisting of A1 to A17, or the sequences complementary thereto, are synthesized directly on the chip (Lockhart et al., supra) or synthesized and then addressed to the chip (Sosnowski et al., supra). Preferably, the oligonucleotides are about 20 nucleotides in length.

CanIon cDNA probes labeled with an appropriate compound, such as biotin, digoxigenin or fluorescent dye, are synthesized from the appropriate mRNA population and then randomly fragmented to an average size of 50 to 100 nucleotides. The probes are then hybridized to the chip. After washing as described in Lockhart et al., supra and application of different electric fields (Sosnowsky et al., 1997), the dyes or labeling compounds are detected and quantified. Duplicate hybridizations are performed. Comparative analysis of the intensity of the signal originating from cDNA probes on the same target oligonucleotide in different cDNA samples indicates a differential expression of CanIon mRNA.

### Methods for Inhibiting the Expression of a CanIon Gene

Other therapeutic compositions according to the present invention comprise advantageously an oligonucleotide fragment of the nucleic sequence of CanIon as an antisense tool or a triple helix tool that inhibits the expression of the corresponding CanIon gene. A preferred fragment of the nucleic sequence of CanIon comprises an allele of at least one of the biallelic markers A1 to A17.

Antisense Approach

Preferred methods for using antisense polynucleotides according to the present invention are the procedures described by Sczakiel et al. (1995).

Preferably, the antisense tools are chosen among polynucleotides (15-200 bp long) that are complementary to the 5′ end of the CanIon mRNA. In another embodiment, a combination of different antisense polynucleotides complementary to different parts of the desired targeted gene are used.

Preferred antisense polynucleotides according to the present invention are complementary to a sequence of the mRNAs of CanIon that contains either the translation initiation codon ATG or a splicing donor or acceptor site.

The antisense nucleic acids should have a length and melting temperature sufficient to permit formation of an intracellular duplex having sufficient stability to inhibit the expression of the CanIon mRNA in the duplex. Strategies for designing antisense nucleic acids suitable for use in gene therapy are disclosed in Green et al. (1986) and Izant and Weintraub (1984), the disclosures of which are incorporated herein by reference.

In some strategies, antisense molecules are obtained by reversing the orientation of the CanIon coding region with respect to a promoter so as to transcribe the opposite strand from that which is normally transcribed in the cell. The antisense molecules may be transcribed using in vitro transcription systems such as those which employ T7 or SP6 polymerase to generate the transcript. Another approach involves transcription of CanIon antisense nucleic acids in vivo by operably linking DNA containing the antisense sequence to a promoter in a suitable expression vector.

Alternatively, suitable antisense strategies are those described by Rossi et al. (1991), in International Application Nos. WO 94/23026, WO 95/04141, WO 92/18522 and in European Patent Application No. EP 0 572 287 A2

An alternative to the antisense technology that is used according to the present invention comprises using ribozymes that will bind to a target sequence via their complementary polynucleotide tail and that will cleave the corresponding RNA by hydrolyzing its target site (namely “hammerhead ribozymes”). Briefly, the simplified cycle of a hammerhead ribozyme comprises (1) sequence specific binding to the target RNA via complementary anti sense sequences; (2) site-specific hydrolysis of the cleavable motif of the target strand; and (3) release of cleavage products, which gives rise to another catalytic cycle. Indeed, the use of long-chain antisense polynucleotide (at least 30 bases long) or ribozymes with long antisense arms are advantageous. A preferred delivery system for antisense ribozyme is achieved by covalently linking these antisense ribozymes to lipophilic groups or to use liposomes as a convenient vector. Preferred antisense ribozymes according to the present invention are prepared as described by Sczakiel et al. (1995), the specific preparation procedures being referred to in said article being herein incorporated by reference.

Triple Helix Approach

The CanIon genomic DNA may also be used to inhibit the expression of the CanIon gene based on intracellular triple helix formation.

Triple helix oligonucleotides are used to inhibit transcription from a genome. They are particularly useful for studying alterations in cell activity when it is associated with a particular gene.

Similarly, a portion of the CanIon genomic DNA can be used to study the effect of inhibiting CanIon transcription within a cell. Traditionally, homopurine sequences were considered the most useful for triple helix strategies. However, homopyrimidine sequences can also inhibit gene expression. Such homopyrimidine oligonucleotides bind to the major groove at homopurine:homopyrimidine sequences. Thus, both types of sequences from the CanIon genomic DNA are contemplated within the scope of this invention.

To carry out gene therapy strategies using the triple helix approach, the sequences of the CanIon genomic DNA are first scanned to identify 10-mer to 20-mer homopyrimidine or homopurine stretches which could be used in triple-helix based strategies for inhibiting CanIon expression. Following identification of candidate homopyrimidine or homopurine stretches, their efficiency in inhibiting CanIon expression is assessed by introducing varying amounts of oligonucleotides containing the candidate sequences into tissue culture cells which express the CanIon gene.

The oligonucleotides can be introduced into the cells using a variety of methods known to those skilled in the art, including but not limited to calcium phosphate precipitation, DEAE-Dextran, electroporation, liposome-mediated transfection or native uptake.

Treated cells are monitored for altered cell function or reduced CanIon expression using techniques such as Northern blotting, RNase protection assays, or PCR based strategies to monitor the transcription levels of the CanIon gene in cells which have been treated with the oligonucleotide.

The oligonucleotides which are effective in inhibiting gene expression in tissue culture cells may then be introduced in vivo using the techniques described above in the antisense approach at a dosage calculated based on the in vitro results, as described in antisense approach.

In some embodiments, the natural (beta) anomers of the oligonucleotide units can be replaced with alpha anomers to render the oligonucleotide more resistant to nucleases. Further, an intercalating agent such as ethidium bromide, or the like, can be attached to the 3′ end of the alpha oligonucleotide to stabilize the triple helix. For information on the generation of oligonucleotides suitable for triple helix formation see Griffin et al. (1989), which is hereby incorporated by this reference.

Pharmaceutical Compositions and Formulations

CanIon-Modulating Compounds

Using the methods disclosed herein, CanIon agonist or antagonist compounds that selectively modulate CanIon activity in vitro and in vivo may be identified. The invention thus encompasses methods of treating schizophrenia, bipolar disorder, or any of the other herein-described diseases or conditions in a patient comprising administering an effective amount of a CanIon-modulating compound. Preferably, said compound is a selective CanIon modulating compound. The compounds identified by the process of the invention include, for example, antibodies having binding specificity for a human CanIon polypeptide. It is also expected that homologues of CanIon may be useful for modulating CanIon-mediated activity and the related physiological condition associated with schizophrenia or bipolar disorder. Generally, it is further expected that assay methods of the present invention based on the role of CanIon in central nervous system disorder may be used to identify compounds capable of intervening in the assay cascade of the invention. In a preferred embodiment, a patient suffering from schizophrenia or bipolar disorder is treated by administering to the patient a pharmaceutical composition comprising a therapeutically effective amount of a CanIon antagonist.

Indications

While CanIon is linked to a genomic region associated with schizophrenia and bipolar disorder, indications involving CanIon may include various central nervous system disorders. Nervous system disorders are expected to have complex genetic bases and often share certain symptoms. In particular, as described herein, indications may include schizophrenia and other psychotic disorders, mood disorders, autism, substance dependence and alcoholism, epilepsy, pain disorders, mental retardation, and other psychiatric diseases including cognitive, anxiety, eating, impulse-control, and personality disorders, as defined with the Diagnosis and Statistical Manual of Mental Disorders fourth edition (DSM-IV) classification. In addition, numerous cardiovascular disorders including angina, hypertension, and arrythmias may also be treated using CanIon modulators, preferably antagonists.

Pharmaceutical Formulations and Routes of Administration

The compounds identified using the methods of the present invention can be administered to a mammal, including a human patient, alone or in pharmaceutical compositions where they are mixed with suitable carriers or excipient(s) at therapeutically effective doses to treat or ameliorate schizophrenia or bipolar disorder related disorders. A therapeutically effective dose further refers to that amount of the compound sufficient to result in amelioration of symptoms as determined by the methods described herein. Preferably, a therapeutically effective dosage is suitable for continued periodic use or administration. Techniques for formulation and administration of the compounds of the instant application may be found in “Remington's Pharmaceutical Sciences,” Mack Publishing Co., Easton, Pa., latest edition.

Routes of Administration

Suitable routes of administration include oral, rectal, transmucosal, or intestinal administration, parenteral delivery, including intramuscular, subcutaneous, intramedullary injections, as well as intrathecal, direct intraventricular, intravenous, intraperitoneal, intranasal or intraocular injections. A particularly useful method of administering compounds for treating central nervous system disease involves surgical implantation of a device for delivering the compound over an extended period of time. Sustained release formulations of the invented medicaments particularly are contemplated.

Composition/Formulation

Pharmaceutical compositions and medicaments for use in accordance with the present invention may be formulated in a conventional manner using one or more physiologically acceptable carriers comprising excipients and auxiliaries. Proper formulation is dependent upon the route of administration chosen.

For injection, the agents of the invention may be formulated in aqueous solutions, preferably in physiologically compatible buffers such as Hanks's solution, Ringer's solution, or physiological saline buffer such as a phosphate or bicarbonate buffer. For transmucosal administration, penetrants appropriate to the barrier to be permeated are used in the formulation. Such penetrants are generally known in the art.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable gaseous propellant, e.g., carbon dioxide. In the case of a pressurized aerosol the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in aqueous vehicles, and may contain formulatory agents such as suspending, stabilizing and/or dispersing agents.

Pharmaceutical formulations for parenteral administration include aqueous solutions of the active compounds in water-soluble form. Aqueous suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder or lyophilized form for constitution with a suitable vehicle, such as sterile pyrogen-free water, before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Additionally, the compounds may be delivered using a sustained-release system, such as semipermeable matrices of solid hydrophobic polymers containing the therapeutic agent. Various sustained release materials have been established and are well known by those skilled in the art. Sustained-release capsules may, depending on their chemical nature, release the compounds for a few weeks up to over 100 days.

Depending on the chemical nature and the biological stability of the therapeutic reagent, additional strategies for protein stabilization may be employed.

The pharmaceutical compositions may also comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Effective Dosage.

Pharmaceutical compositions suitable for use in the present invention include compositions wherein the active ingredients are contained in an effective amount to achieve their intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays, and a dose can be formulated in animal models. Such information can be used to more accurately determine useful doses in humans.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the LD50, (the dose lethal to 50% of the test population) and the ED50 (the dose therapeutically effective in 50% of the population). The dose ratio between toxic and therapeutic effects is the therapeutic index and it can be expressed as the ratio between LD50 and ED50. Compounds which exhibit high therapeutic indices are preferred.

The data obtained from these cell culture assays and animal studies can be used in formulating a range of dosage for use in human. The dosage of such compounds lies preferably within a range of circulating concentrations that include the ED50, with little or no toxicity. The dosage may vary within this range depending upon the dosage form employed and the route of administration utilized. The exact formulation, route of administration and dosage can be chosen by the individual physician in view of the patients condition. (See, e.g., Fingl et al., 1975, in “The Pharmacological Basis of Therapeutics”, Ch. 1).

### Computer-Related Embodiments

As used herein the term “nucleic acid codes of the invention” encompass the nucleotide sequences comprising, consisting essentially of, or consisting of any one of the following: a) a contiguous span of at least 12, 15, 18, 20, 25, 30, 35, 40, 50, 60, 70, 80, 90, 100, 150, 200, 500, 1000, 2000, 3000, 4000, 5000 or 10000 nucleotides of SEQ ID No 1 to 3; b) a contiguous span of at least 12, 15, 18, 20, 25, 30, 35, 40, 50, 60, 70, 80, 90, 100, 150, 200, 500, 1000, 2000, 3000, 4000, 5000 or 6000 nucleotides of SEQ ID No 4 or the complements thereof; c) a contiguous span of at least 12, 15, 18, 20, 25, 30, 35, 40, 50, 60, 70, 80, 90, 100, 150, 200, 500, 1000, 2000, 3000, 4000, 5000 or 10000 nucleotides of SEQ ID Nos 1 to 3 wherein said contiguous span comprises a biallelic marker selected from the group consisting of A1 to A17; d) a contiguous span of at least 12, 15, 18, 20, 25, 30, 35, 40, 50, 60, 70, 80, 90, 100, 150, 200, 300 or 400 nucleotides of SEQ ID No 6; e) a contiguous span of at least 12, 15, 18, 20, 25, 30, 35, 40, 50, 60, 70, 80, 90, 100, 150, 200, 300 or 400 nucleotides of SEQ ID No 6 wherein said contiguous span comprises biallelic marker A18; and, f) a nucleotide sequence complementary to any one of the preceding nucleotide sequences.

The “nucleic acid codes of the invention” further encompass nucleotide sequences homologous to: a) a contiguous span of at least 12, 15, 18, 20, 25, 30, 35, 40, 50, 60, 70, 80, 90, 100, 150, 200, 500, 1000, 2000, 3000, 4000, 5000 or 10000 nucleotides of SEQ ID No 1 to 3; b) a contiguous span of at least 12, 15, 18, 20, 25, 30, 35, 40, 50, 60, 70, 80, 90, 100, 150, 200, 500, 1000, 2000, 3000, 4000, 5000 or 6000 nucleotides of SEQ ID No 4; c) a contiguous span of at least 12, 15, 18, 20, 25, 30, 35, 40, 50, 60, 70, 80, 90, 100, 150, 200, 300 or 400 nucleotides of SEQ ID No 6; and, d) sequences complementary to any one of the preceding sequences. Homologous sequences refer to a sequence having at least 99%, 98%, 97%, 96%, 95%, 90%, 85%, 80%, or 75% homology to these contiguous spans. Homology may be determined using any method described herein, including BLAST2N with the default parameters or with any modified parameters. Homologous sequences also may include RNA sequences in which uridines replace the thymines in the nucleic acid codes of the invention. It will be appreciated that the nucleic acid codes of the invention can be represented in the traditional single character format (See the inside back cover of Stryer, Lubert. Biochemistry, 3^{rd} edition. W. H Freeman & Co., New York.) or in any other format or code which records the identity of the nucleotides in a sequence.

As used herein the term “polypeptide codes of the invention” encompass the polypeptide sequences comprising a contiguous span of at least 6, 8, 10, 12, 15, 20, 25, 30, 40, 50, 100, 150, 200, 300, 400, 500, 700, 1000, 1200, 1400, 1600 or 1700 amino acids of SEQ ID No 5. In preferred embodiments, said contiguous span includes at least 1, 2, 3, 5 or 10 of the amino acid positions 277, 338, 574, 678, 680, 683, 691, 692, 695, 696, 697, 894, 1480, 1481, 1483, 1484, 1485, 1630, 1631, 1632, 1636, 1660, 1667, 1707, 1709 of SEQ ID No 5. It will be appreciated that the polypeptide codes of the invention can be represented in the traditional single character format or three letter format (See the inside back cover of Stryer, Lubert. Biochemistry, 3^{rd} edition. W. H Freeman & Co., New York.) or in any other format or code which records the identity of the polypeptides in a sequence.

It will be appreciated by those skilled in the art that the nucleic acid codes of the invention and polypeptide codes of the invention can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the nucleic acid codes of the invention, or one or more of the polypeptide codes of the invention. Another aspect of the present invention is a computer readable medium having recorded thereon at least 2, 5, 10, 15, 20, 25, 30, or 50 nucleic acid codes of the invention. Another aspect of the present invention is a computer readable medium having recorded thereon at least 2, 5, 10, 15, 20, 25, 30, or 50 polypeptide codes of the invention.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

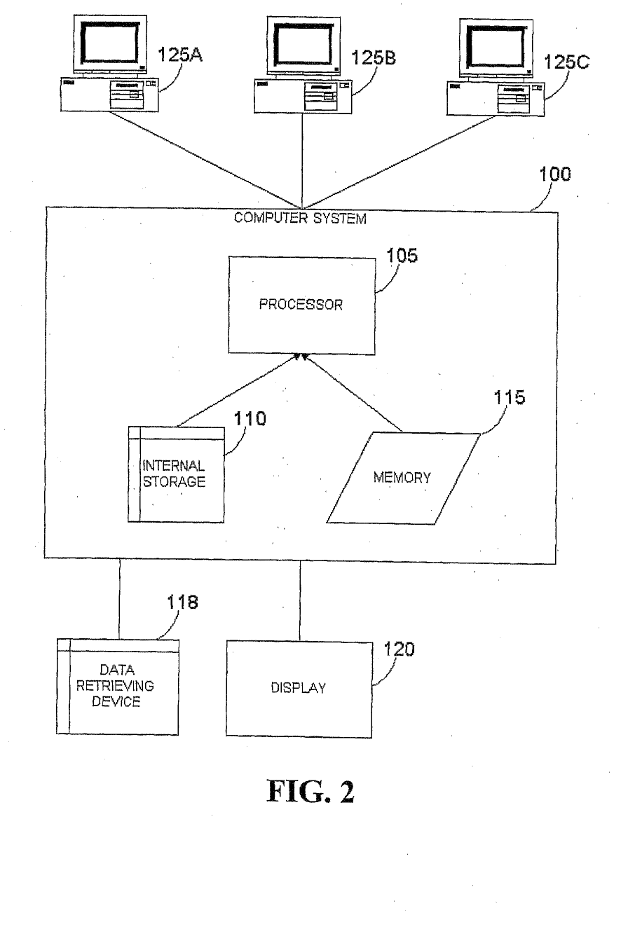

Embodiments of the present invention include systems, particularly computer systems which store and manipulate the sequence information described herein. One example of a computer system 100 is illustrated in block diagram form in FIG. 2. As used herein, “a computer system” refers to the hardware components, software components, and data storage components used to analyze the nucleotide sequences of the nucleic acid codes of the invention or the amino acid sequences of the polypeptide codes of the invention. In one embodiment, the computer system 100 is a Sun Enterprise 1000 server (Sun Microsystems, Palo Alto, Calif.). The computer system 100 preferably includes a processor for processing, accessing and manipulating the sequence data. The processor 105 can be any well-known type of central processing unit, such as the Pentium III from Intel Corporation, or similar processor from Sun, Motorola, Compaq or International Business Machines.

Preferably, the computer system 100 is a general purpose system that comprises the processor 105 and one or more internal data storage components 110 for storing data, and one or more data retrieving devices for retrieving the data stored on the data storage components. A skilled artisan can readily appreciate that any one of the currently available computer systems are suitable.

In one particular embodiment, the computer system 100 includes a processor 105 connected to a bus which is connected to a main memory 115 (preferably implemented as RAM) and one or more internal data storage devices 110, such as a hard drive and/or other computer readable media having data recorded thereon. In some embodiments, the computer system 100 further includes one or more data retrieving device 118 for reading the data stored on the internal data storage devices 110.

The data retrieving device 118 may represent, for example, a floppy disk drive, a compact disk drive, a magnetic tape drive, etc. In some embodiments, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data retrieving device.

The computer system 100 includes a display 120 which is used to display output to a computer user. It should also be noted that the computer system 100 can be linked to other computer systems 125a-c in a network or wide area network to provide centralized access to the computer system 100.

Software for accessing and processing the nucleotide sequences of the nucleic acid codes of the invention or the amino acid sequences of the polypeptide codes of the invention (such as search tools, compare tools, and modeling tools etc.) may reside in main memory 115 during execution.

In some embodiments, the computer system 100 may further comprise a sequence comparer for comparing the above-described nucleic acid codes of the invention or the polypeptide codes of the invention stored on a computer readable medium to reference nucleotide or polypeptide sequences stored on a computer readable medium. A “sequence comparer” refers to one or more programs which are implemented on the computer system 100 to compare a nucleotide or polypeptide sequence with other nucleotide or polypeptide sequences and/or compounds including but not limited to peptides, peptidomimetics, and chemicals stored within the data storage means. For example, the sequence comparer may compare the nucleotide sequences of nucleic acid codes of the invention or the amino acid sequences of the polypeptide codes of the invention stored on a computer readable medium to reference sequences stored on a computer readable medium to identify homologies, motifs implicated in biological function, or structural motifs. The various sequence comparer programs identified elsewhere in this patent specification are particularly contemplated for use in this aspect of the invention.

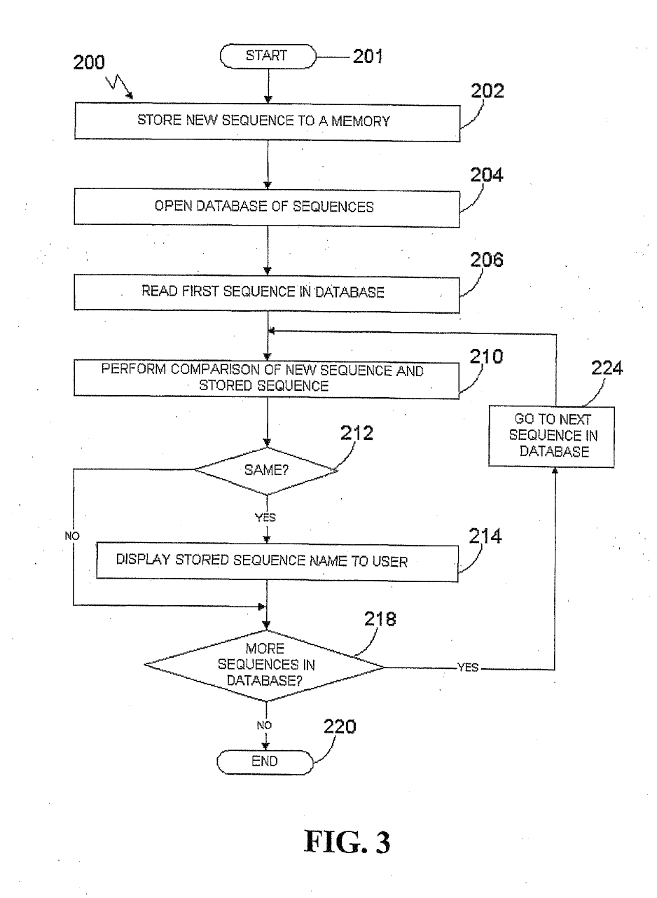

FIG. 3 is a flow diagram illustrating one embodiment of a process 200 for comparing a new nucleotide or protein sequence with a database of sequences in order to determine the homology levels between the new sequence and the sequences in the database. The database of sequences can be a private database stored within the computer system 100, or a public database such as GENBANK, PIR OR SWISSPROT that is available through the Internet.

The process 200 begins at a start state 201 and then moves to a state 202 wherein the new sequence to be compared is stored to a memory in a computer system 100. As discussed above, the memory could be any type of memory, including RAM or an internal storage device.

The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide or protein sequences, even if they are not identical. For example, gaps can be introduced into one sequence in order to raise the homology level between the two tested sequences. The parameters that control whether gaps or other features are introduced into a sequence during comparison are normally entered by the user of the computer system.

Once a comparison of the two sequences has been performed at the state 210, a determination is made at a decision state 210 whether the two sequences are the same. Of course, the term “same” is not limited to sequences that are absolutely identical. Sequences that are within the homology parameters entered by the user will be marked as “same” in the process 200.

If a determination is made that the two sequences are the same, the process 200 moves to a state 214 wherein the name of the sequence from the database is displayed to the user. This state notifies the user that the sequence with the displayed name fulfills the homology constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database.

It should be noted that if a determination had been made at the decision state 212 that the sequences were not homologous, then the process 200 would move immediately to the decision state 218 in order to determine if any other sequences were available in the database for comparison.

Accordingly, one aspect of the present invention is a computer system comprising a processor, a data storage device having stored thereon a nucleic acid code of the invention or a polypeptide code of the invention, a data storage device having retrievably stored thereon reference nucleotide sequences or polypeptide sequences to be compared to the nucleic acid code of the invention or polypeptide code of the invention and a sequence comparer for conducting the comparison. The sequence comparer may indicate a homology level between the sequences compared or identify motifs implicated in biological function and structural motifs in the nucleic acid code of the invention and polypeptide codes of the invention or it may identify structural motifs in sequences which are compared to these nucleic acid codes and polypeptide codes. In some embodiments, the data storage device may have stored thereon the sequences of at least 2, 5, 10, 15, 20, 25, 30, or 50 of the nucleic acid codes of the invention or polypeptide codes of the invention.

Another aspect of the present invention is a method for determining the level of homology between a nucleic acid code of the invention and a reference nucleotide sequence, comprising the steps of reading the nucleic acid code and the reference nucleotide sequence through the use of a computer program which determines homology levels and determining homology between the nucleic acid code and the reference nucleotide sequence with the computer program. The computer program may be any of a number of computer programs for determining homology levels, including those specifically enumerated herein, including BLAST2N with the default parameters or with any modified parameters. The method may be implemented using the computer systems described above. The method may also be performed by reading 2, 5, 10, 15, 20, 25, 30, or 50 of the above described nucleic acid codes of the invention through the use of the computer program and determining homology between the nucleic acid codes and reference nucleotide sequences.

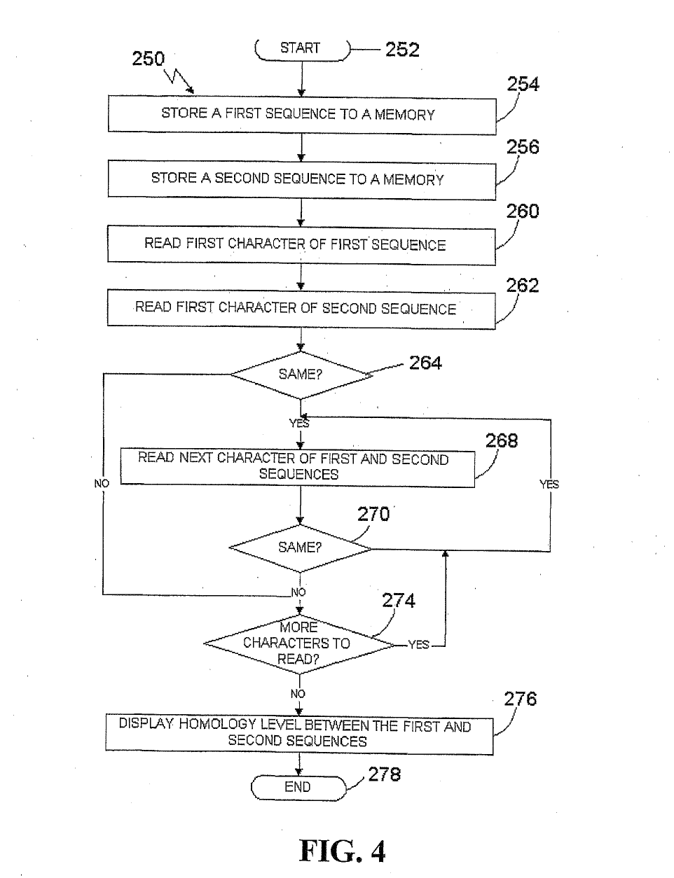

FIG. 4 is a flow diagram illustrating one embodiment of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U. If the sequence is a protein sequence, then it should be in the single letter amino acid code so that the first and sequence sequences can be easily compared.

A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read.

If there aren't any more characters to read, then the process 250 moves to a state 276 wherein the level of homology between the first and second sequences is displayed to the user. The level of homology is determined by calculating the proportion of characters between the sequences that were the same out of the total number of sequences in the first sequence. Thus, if every character in a first 100 nucleotide sequence aligned with a every character in a second sequence, the homology level would be 100%.

Alternatively, the computer program may be a computer program which compares the nucleotide sequences of the nucleic acid codes of the present invention, to reference nucleotide sequences in order to determine whether the nucleic acid code of the invention differs from a reference nucleic acid sequence at one or more positions. Optionally such a program records the length and identity of inserted, deleted or substituted nucleotides with respect to the sequence of either the reference polynucleotide or the nucleic acid code of the invention. In one embodiment, the computer program may be a program which determines whether the nucleotide sequences of the nucleic acid codes of the invention contain one or more single nucleotide polymorphisms (SNP) with respect to a reference nucleotide sequence. These single nucleotide polymorphisms may each comprise a single base substitution, insertion, or deletion.

Another aspect of the present invention is a method for determining the level of homology between a polypeptide code of the invention and a reference polypeptide sequence, comprising the steps of reading the polypeptide code of the invention and the reference polypeptide sequence through use of a computer program which determines homology levels and determining homology between the polypeptide code and the reference polypeptide sequence using the computer program.

Accordingly, another aspect of the present invention is a method for determining whether a nucleic acid code of the invention differs at one or more nucleotides from a reference nucleotide sequence comprising the steps of reading the nucleic acid code and the reference nucleotide sequence through use of a computer program which identifies differences between nucleic acid sequences and identifying differences between the nucleic acid code and the reference nucleotide sequence with the computer program. In some embodiments, the computer program is a program which identifies single nucleotide polymorphisms The method may be implemented by the computer systems described above and the method illustrated in FIG. 4. The method may also be performed by reading at least 2, 5, 10, 15, 20, 25, 30, or 50 of the nucleic acid codes of the invention and the reference nucleotide sequences through the use of the computer program and identifying differences between the nucleic acid codes and the reference nucleotide sequences with the computer program.

In other embodiments the computer based system may further comprise an identifier for identifying features within the nucleotide sequences of the nucleic acid codes of the invention or the amino acid sequences of the polypeptide codes of the invention.

An “identifier” refers to one or more programs which identifies certain features within the above-described nucleotide sequences of the nucleic acid codes of the invention or the amino acid sequences of the polypeptide codes of the invention. In one embodiment, the identifier may comprise a program which identifies an open reading frame in the cDNAs codes of the invention.

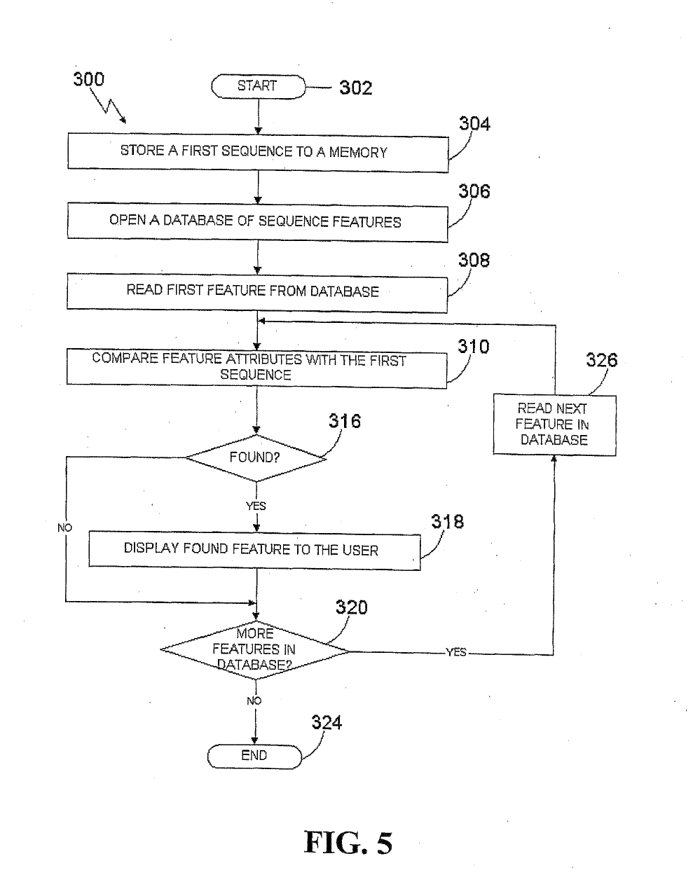

FIG. 5 is a flow diagram illustrating one embodiment of an identifier process 300 for detecting the presence of a feature in a sequence. The process 300 begins at a start state 302 and then moves to a state 304 wherein a first sequence that is to be checked for features is stored to a memory 115 in the computer system 100. The process 300 then moves to a state 306 wherein a database of sequence features is opened. Such a database would include a list of each feature's attributes along with the name of the feature. For example, a feature name could be “Initiation Codon” and the attribute would be “ATG”. Another example would be the feature name “TAATAA Box” and the feature attribute would be “TAATAA”. An example of such a database is produced by the University of Wisconsin Genetics Computer Group (www.gcg.com).

Once the database of features is opened at the state 306, the process 300 moves to a state 308 wherein the first feature is read from the database. A comparison of the attribute of the first feature with the first sequence is then made at a state 310. A determination is then made at a decision state 316 whether the attribute of the feature was found in the first sequence. If the attribute was found, then the process 300 moves to a state 318 wherein the name of the found feature is displayed to the user.

The process 300 then moves to a decision state 320 wherein a determination is made whether move features exist in the database. If no more features do exist, then the process 300 terminates at an end state 324. However, if more features do exist in the database, then the process 300 reads the next sequence feature at a state 326 and loops back to the state 310 wherein the attribute of the next feature is compared against the first sequence.

It should be noted, that if the feature attribute is not found in the first sequence at the decision state 316, the process 300 moves directly to the decision state 320 in order to determine if any more features exist in the database.

In another embodiment, the identifier may comprise a molecular modeling program which determines the 3-dimensional structure of the polypeptides codes of the invention. In some embodiments, the molecular modeling program identifies target sequences that are most compatible with profiles representing the structural environments of the residues in known three-dimensional protein structures. (See, e.g., U.S. Pat. No. 5,436,850). In another technique, the known three-dimensional structures of proteins in a given family are superimposed to define the structurally conserved regions in that family. This protein modeling technique also uses the known three-dimensional structure of a homologous protein to approximate the structure of the polypeptide codes of the invention. (See e.g., U.S. Pat. No. 5,557,535). Conventional homology modeling techniques have been used routinely to build models of proteases and antibodies. (Sowdhamini et al., (1997)). Comparative approaches can also be used to develop three-dimensional protein models when the protein of interest has poor sequence identity to template proteins. In some cases, proteins fold into similar three-dimensional structures despite having very weak sequence identities. For example, the three-dimensional structures of a number of helical cytokines fold in similar three-dimensional topology in spite of weak sequence homology.

The recent development of threading methods now enables the identification of likely folding patterns in a number of situations where the structural relatedness between target and template(s) is not detectable at the sequence level. Hybrid methods, in which fold recognition is performed using Multiple Sequence Threading (MST), structural equivalencies are deduced from the threading output using a distance geometry program DRAGON to construct a low resolution model, and a full-atom representation is constructed using a molecular modeling package such as QUANTA.

According to this 3-step approach, candidate templates are first identified by using the novel fold recognition algorithm MST, which is capable of performing simultaneous threading of multiple aligned sequences onto one or more 3-D structures. In a second step, the structural equivalencies obtained from the MST output are converted into interresidue distance restraints and fed into the distance geometry program DRAGON, together with auxiliary information obtained from secondary structure predictions. The program combines the restraints in an unbiased manner and rapidly generates a large number of low resolution model confirmations. In a third step, these low resolution model confirmations are converted into full-atom models and subjected to energy minimization using the molecular modeling package QUANTA (See e.g., Aszódi et al., (1997)).

The results of the molecular modeling analysis may then be used in rational drug design techniques to identify agents which modulate the activity of the polypeptide codes of the invention.

Accordingly, another aspect of the present invention is a method of identifying a feature within the nucleic acid codes of the invention or the polypeptide codes of the invention comprising reading the nucleic acid code(s) or the polypeptide code(s) through the use of a computer program which identifies features therein and identifying features within the nucleic acid code(s) or polypeptide code(s) with the computer program. In one embodiment, computer program comprises a computer program which identifies open reading frames. In a further embodiment, the computer program identifies structural motifs in a polypeptide sequence. In another embodiment, the computer program comprises a molecular modeling program. The method may be performed by reading a single sequence or at least 2, 5, 10, 15, 20, 25, 30, or 50 of the nucleic acid codes of the invention or the polypeptide codes of the invention through the use of the computer program and identifying features within the nucleic acid codes or polypeptide codes with the computer program.

The nucleic acid codes of the invention or the polypeptide codes of the invention may be stored and manipulated in a variety of data processor programs in a variety of formats. For example, they may be stored as text in a word processing file, such as MicrosoftWORD or WORDPERFECT or as an ASCII file in a variety of database programs familiar to those of skill in the art, such as DB2, SYBASE, or ORACLE. In addition, many computer programs and databases may be used as sequence comparers, identifiers, or sources of reference nucleotide or polypeptide sequences to be compared to the nucleic acid codes of the invention or the polypeptide codes of the invention. The following list is intended not to limit the invention but to provide guidance to programs and databases which are useful with the nucleic acid codes of the invention or the polypeptide codes of the invention. The programs and databases which may be used include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, 1990), FASTA (Pearson and Lipman, 1988), FASTDB (Brutlag et al., 1990), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.) the EMBL/Swissprotein database, the MDL Inc.), SeqFold (Molecular Simulations Inc.), the EML/Swissprotein database, the MDL Available Chemicals Directory database, the MDL Drug Data Report data base, the Comprehensive Medicinal Chemistry database, Derwents's World Drug Index database, the BioByteMasterFile database, the Genbank database, and the Genseqn database. Many other programs and data bases would be apparent to one of skill in the art given the present disclosure.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

Throughout this application, various publications, patents and published patent applications are cited. The disclosures of these publications, patents and published patent specification referenced in this application are hereby incorporated by reference into the present disclosure to more fully describe the sate of the art to which this invention pertains.

### EXAMPLES

### Example 1

### Identification of Biallelic Markers

### DNA Extraction

Donors were unrelated and healthy. They presented a sufficient diversity for being representative of a French heterogeneous population. The DNA from 100 individuals was extracted and tested for the detection of the biallelic markers.

30 ml of peripheral venous blood were taken from each donor in the presence of EDTA. Cells (pellet) were collected after centrifugation for 10 minutes at 2000 rpm. Red cells were lysed by a lysis solution (50 ml final volume: 10 mM Tris pH7.6; 5 mM MgCl₂; 10 mM NaCl). The solution was centrifuged (10 minutes, 2000 rpm) as many times as necessary to eliminate the residual red cells present in the supernatant, after resuspension of the pellet in the lysis solution.

The pellet of white cells was lysed overnight at 42° C. with 3.7 ml of lysis solution composed of:
3 ml TE 10-2 (Tris-HCl 10 mM, EDTA 2 mM)/NaCl 0 4 M
200 μl SDS 10%
500 μl K-proteinase (2 mg K-proteinase in TE 10-2/NaCl 0.4 M).

For the extraction of proteins, 1 ml saturated NaCl (6M) (1/3.5 v/v) was added. After vigorous agitation, the solution was centrifuged for 20 minutes at 10000 rpm.

For the precipitation of DNA, 2 to 3 volumes of 100% ethanol were added to the previous supernatant, and the solution was centrifuged for 30 minutes at 2000 rpm. The DNA solution was rinsed three times with 70% ethanol to eliminate salts, and centrifuged for 20 minutes at 2000 rpm. The pellet was dried at 37° C., and resuspended in 1 ml TE 10-1 or 1 ml water. The DNA concentration was evaluated by measuring the OD at 260 nm (1 unit OD=50 μg/ml DNA).

To determine the presence of proteins in the DNA solution, the OD 260/OD 280 ratio was determined. Only DNA preparations having a OD 260/OD 280 ratio between 1.8 and 2 were used in the subsequent examples described below.

The pool was constituted by mixing equivalent quantities of DNA from each individual.

### Example 2

### Identification of Biallelic Markers

### Amplification of Genomic DNA by PCR

The amplification of specific genomic sequences of the DNA samples of example 1 was carried out on the pool of DNA obtained previously. In addition, 50 individual samples were similarly amplified.

PCR assays were performed using the following protocol:

| | | |
|---|---|---|
| Final volume | 25 | μl |
| DNA | 2 | ng/μl |
| MgCl₂ | 2 | mM |
| dNTP (each) | 200 | μM |
| primer (each) | 2.9 | ng/μl |
| Ampli Taq Gold DNA polymerase | 0.05 | unit/μl |

| | |
|---|---|
| PCR buffer (10x = 0.1 M TrisHCl pH8.3 0.5M KCl) | 1x |

Each pair of first primers was designed using the sequence information of the CanIon gene disclosed herein and the OSP software (Hillier & Green, 1991). This first pair of primers was about 20 nucleotides in length and had the sequences disclosed in Table 1 in the columns labeled PU and RP.

TABLE 1

| | | | | Position range | | | Complementary | |
|---|---|---|---|---|---|---|---|---|
| | Position range of | | | of amplification | | | position range of | |
| | the amplicon in | | Primer | primer in | | Primer | amplification primer | |
| Amplicon | SEQ ID 1 | | name | SEQ ID No 1 | | name | in SEQ ID No 1 | |
| 99-62626 | 12343 | 12810 | B1 | 12343 | 12363 | C1 | 12793 | 12810 |
| 99-62632 | 13814 | 14296 | B2 | 13814 | 13832 | C2 | 14279 | 14296 |
| 99-62633 | 24863 | 25396 | B3 | 24863 | 24881 | C3 | 25378 | 25396 |
| 99-62611 | 69198 | 69650 | B4 | 69198 | 69218 | C4 | 69632 | 69650 |
| 99-62605 | 73005 | 73483 | B5 | 73005 | 73022 | C5 | 73466 | 73483 |
| 99-62635 | 79808 | 80334 | B6 | 79808 | 79826 | C6 | 80314 | 80334 |
| | | | | Position range | | | Complementary | |
| | Position range of | | | of amplification | | | position range of | |
| | the amplicon in | | rimer | primer in | | Primer | amplification primer | |
| Amplicon | SEQ ID 2 | | name | SEQ ID No 2 | | name | in SEQ ID No 2 | |
| 99-79335 | 51031 | 51559 | B7 | 51031 | 51051 | C7 | 51539 | 51559 |
| 99-79336 | 60925 | 61374 | B8 | 60925 | 60945 | C8 | 61354 | 61374 |
| 99-79338 | 80271 | 80720 | B9 | 80271 | 80290 | C9 | 80700 | 80720 |
| 99-79339 | 91037 | 91486 | B10 | 91037 | 91056 | C10 | 91466 | 91486 |
| 99-79314 | 100285 | 100784 | B11 | 100285 | 100305 | C11 | 100764 | 100784 |
| 99-79316 | 106568 | 107020 | B12 | 106568 | 106585 | C12 | 107000 | 107020 |
| 99-79322 | 165864 | 166401 | B13 | 165864 | 165884 | C13 | 166381 | 166401 |
| 99-79306 | 235713 | 236210 | B14 | 235713 | 235732 | C14 | 236190 | 236210 |
| | | | | Position range | | | Complementary | |
| | Position range of | | | of amplification | | | position range of | |
| | the amplicon in | | Primer | primer in | | Primer | amplification primer | |
| Amplicon | SEQ ID 3 | | name | SEQ ID No 3 | | name | in SEQ ID No 3 | |
| 99-79310 | 31618 | 32100 | B15 | 31618 | 31635 | C15 | 32080 | 32100 |
| 99-79311 | 42324 | 42723 | B16 | 42324 | 42344 | C16 | 42704 | 42723 |

| | | | | |
|---|---|---|---|---|
| | | Position range of | | Complementary |
| | | amplification | | position range of |
| | Primer | primer in | Primer | amplification primer in |
| Amplicon | name | SEQ ID No 6 | name | SEQ ID No 6 |

| | | | | | | |
|---|---|---|---|---|---|---|
| 99-62617 | B17 | 1 | 20 | C17 | 435 | 453 |

Preferably, the primers contained a common oligonucleotide tail upstream of the specific bases targeted for amplification which was useful for sequencing.

Primers PU contain the following additional PU 5′ sequence: TGTAAAACGACGGCCAGT; primers RP contain the following RP 5′ sequence: CAGGAAACAGCTATGACC. The primer containing the additional PU 5′ sequence is listed in SEQ ID No 7. The primer containing the additional RP 5′ sequence is listed in SEQ ID No 8.

The synthesis of these primers was performed following the phosphoramidite method, on a GENSET UFPS 24.1 synthesizer.

DNA amplification was performed on a Genius II thermocycler. After heating at 95° C. for 10 min, 40 cycles were performed. Each cycle comprised: 30 sec at 95° C., 54° C. for 1 min, and 30 sec at 72° C. For final elongation, 10 min at 72° C. ended the amplification. The quantities of the amplification products obtained were determined on 96-well microtiter plates, using a fluorometer and Picogreen as intercalant agent (Molecular Probes).

### Example 3

### Identification of Biallelic Markers

### Sequencing of Amplified Genomic DNA And Identification of Polymorphisms

The sequencing of the amplified DNA obtained in example 2 was carried out on ABI 377 sequencers. The sequences of the amplification products were determined using automated dideoxy terminator sequencing reactions with a dye terminator cycle sequencing protocol. The products of the sequencing reactions were run on sequencing gels and the sequences were determined using gel image analysis (ABI Prism DNA Sequencing Analysis software (2.1.2 version)).

The sequence data were further evaluated to detect the presence of biallelic markers within the amplified fragments. The polymorphism search was based on the presence of superimposed peaks in the electrophoresis pattern resulting from different bases occurring at the same position as described previously.

In the 17 fragments of amplification, 18 biallelic markers were detected. The localization of these biallelic markers are as shown in Table 2.

TABLE 2

| | | | | | BM position | |
|---|---|---|---|---|---|---|
| | | | Polymorphism | | in SEQ ID | |
| Amplicon | BM | Marker Name | all1 | all2 | No 1 | No 4 |
| 99-62626 | A1 | 99-62626-168 | | | 12642 | |
| 99-62632 | A2 | 99-62632-275 | | | 14088 | |
| 99-62633 | A3 | 99-62633-409 | | | 24981 | |
| 99-62611 | A4 | 99-62611-51 | | | 69248 | |
| 99-62605 | A5 | 99-62605-56 | | | 73428 | |
| 99-62635 | A6 | 99-62635-443 | | | 80250 | |
| | | | | | BM position | |
| | | | Polymorphism | | in SEQ ID | |
| Amplicon | BM | Marker Name | all1 | all2 | No 2 | No 4 |
| 99-79335 | A7 | 99-79335-60 | | | 51090 | |
| 99-79336 | A8 | 99-79336-369 | | | 61293 | |
| 99-79338 | A9 | 99-79338-332 | | | 80602 | |
| 99-79314 | A10 | 99-79314-201 | | | 100485 | |
| 99-79314 | A11 | 99-79314-225 | | | 100509 | |
| 99-79316 | A12 | 99-79316-158 | | | 106725 | 1658 |
| 99-7932 | A13 | 99-79322-224 | | | 166087 | |
| 99-79322 | A14 | 99-79322-473 | | | 166336 | |
| 99-79306 | A15 | 99-79306-182 | | | 235894 | |
| | | | | | BM position | |
| | | | Polymorphism | | in SEQ ID | |
| Amplicon | BM | Marker Name | all1 | all2 | No 3 | No 4 |
| 99-79310 | A16 | 99-79310-29 | | | 31646 | 4481 |
| 99-79311 | A17 | 99-79311-50 | | | 42373 | |

| | | | | | BM position |
|---|---|---|---|---|---|
| | | | Polymorphism | | in SEQ ID |
| Amplicon | BM | Marker Name | all1 | all2 | No 6 |
| 99-62617 | A18 | 99-62617-105 | | | 105 |

BM refers to “biallelic marker”. All1 and all2 refer respectively to allele 1 and allele 2 of the biallelic marker.

TABLE 3

| | | | |
|---|---|---|---|
| | | Position range | |
| | | of probes in | |
| BM | Marker Name | SEQ ID No 1 | Probes |

| | | | | |
|---|---|---|---|---|
| A1 | 99-62626-168 | 12630 | 12654 | P1 |
| A2 | 99-62632-275 | 14076 | 14100 | P2 |
| A3 | 99-62633-409 | 24969 | 24993 | P3 |
| A4 | 99-62611-51 | 69236 | 69260 | P4 |
| A5 | 99-62605-56 | 73416 | 73440 | P5 |
| A6 | 99-62635-443 | 80238 | 80262 | P6 |

| | | | |
|---|---|---|---|
| | | Position range | |
| | | of probes in | |
| BM | Marker Name | SEQ ID No 2 | Probes |

| | | | | |
|---|---|---|---|---|
| A7 | 99-79335-60 | 51078 | 51102 | P7 |
| A8 | 99-79336-369 | 61281 | 61305 | P8 |
| A9 | 99-79338-332 | 80590 | 80614 | P9 |
| A10 | 99-79314-201 | 100473 | 100497 | P10 |
| A11 | 99-79314-225 | 100497 | 100521 | P11 |
| A12 | 99-79316-158 | 106713 | 106737 | P12 |
| A13 | 99-79322-224 | 166075 | 166099 | P13 |
| A14 | 99-79322-473 | 166324 | 166348 | P14 |
| A15 | 99-79306-182 | 235882 | 235906 | P15 |

| | | | |
|---|---|---|---|
| | | Position range | |
| | | of probes in | |
| BM | Marker Name | SEQ ID No 3 | Probes |

| | | | | |
|---|---|---|---|---|
| A16 | 99-79310-29 | 31634 | 31658 | P16 |
| A17 | 99-79311-50 | 42361 | 42385 | P17 |

| | | | |
|---|---|---|---|
| | | Position range | |
| | | of probes in | |
| BM | Marker Name | SEQ ID No 6 | Probes |

| | | | | |
|---|---|---|---|---|
| A18 | 99-62617-105 | 93 | 117 | P18 |

### Example 4

### Validation of the Polymorphisms Through Microsequencing

The biallelic markers identified in example 3 were further confirmed and their respective frequencies were determined through microsequencing. Microsequencing was carried out for each individual DNA sample described in Example 1.

Amplification from genomic DNA of individuals was performed by PCR as described above for the detection of the biallelic markers with the same set of PCR primers (Table 1).

The preferred primers used in microsequencing were about 19 nucleotides in length and hybridized just upstream of the considered polymorphic base. According to the invention, the primers used in microsequencing are detailed in Table 4.

TABLE 4

| | | | Position range of | | | Complementary position | |
|---|---|---|---|---|---|---|---|
| | | | microsequencing | | | range of microsequencing | |
| | Biallelic | | primer mis 1 | | | primer mis. 2 in | |
| Marker Name | Marker | Mis. 1 | in SEQ ID No 1 | | Mis. 2 | SEQ ID No 1 | |
| 99-62626-168 | A1 | D1 | 12623 | 12641 | E1 | 12643 | 12661 |
| 99-62632-275 | A2 | D2 | 14069 | 14087 | E2 | 14089 | 14107 |
| 99-62633-409 | A3 | D3 | 24962 | 24980 | E3 | 24982 | 25000 |
| 99-62611-51 | A4 | D4 | 69229 | 69247 | E4 | 69249 | 69267 |
| 99-62605-56 | A5 | D5 | 73409 | 73427 | E5 | 73429 | 73447 |
| 99-62635-443 | A6 | D6 | 80231 | 80249 | E6 | 80251 | 80269 |
| | | | Position range of | | | Complementary position | |
| | | | microsequencing | | | range of microsequencing | |
| | Biallelic | | primer mis 1 | | | primer mis. 2 in | |
| Marker Name | Marker | Mis. 1 | in SEQ ID No 2 | | Mis. 2 | SEQ ID No 2 | |
| 99-79335-60 | A7 | D7 | 51071 | 51089 | E7 | 51091 | 51109 |
| 99-79336-369 | A8 | D8 | 61274 | 61292 | E8 | 61294 | 61312 |
| 99-79338-332 | A9 | D9 | 80583 | 80601 | E9 | 80603 | 80621 |
| 99-79314-201 | A10 | D10 | 100466 | 100484 | E10 | 100486 | 100504 |
| 99-79314-225 | A11 | D11 | 100490 | 100508 | E11 | 100510 | 100528 |
| 99-79316-158 | A12 | D12 | 106706 | 106724 | E12 | 106726 | 106744 |
| 99-79322-224 | A13 | D13 | 166068 | 166086 | E13 | 166088 | 166106 |
| 99-79322-473 | A14 | D14 | 166317 | 166335 | E14 | 166337 | 166355 |
| 99-79306-182 | A15 | D15 | 235875 | 235893 | E15 | 235895 | 235913 |
| | | | Position range of | | | Complementary position | |
| | | | microsequencing | | | range of microsequencing | |
| | Biallelic | | primer mis 1 | | | primer mis. 2 in | |
| Marker Name | Marker | Mis. 1 | in SEQ ID No 3 | | Mis. 2 | SEQ ID No 3 | |
| 99-79310-29 | A16 | D16 | 31627 | 31645 | E16 | 31647 | 31665 |
| 99-79311-50 | A17 | D17 | 42354 | 42372 | E17 | 42374 | 42392 |
| | | | Position range of | | | Complementary position | |
| | | | microsequencing | | | range of microsequencing | |
| | Biallelic | | primer mis 1 | | | primer mis. 2 in | |
| Marker Name | Marker | Mis. 1 | in SEQ ID No 6 | | Mis. 2 | SEQ ID No 6 | |
| 99-62617-105 | A18 | D18 | 86 | 104 | E18 | 106 | 124 |

Mis 1 and M is 2 respectively refer to microsequencing primers which hybridiz with the non-coding strand of the CanIon gene or with the coding strand of the CanIon gene.

The microsequencing reaction was performed as follows:

After purification of the amplification products, the microsequencing reaction mixture was prepared by adding, in a 20 μl final volume: 10 μmol microsequencing oligonucleotide, 1 U Thermosequenase (Amersham E79000G), 1.25 μl Thermosequenase buffer (260 mM Tris HCl pH 9.5, 65 mM MgCl₂), and the two appropriate fluorescent ddNTPs (Perkin Elmer, Dye Terminator Set 401095) complementary to the nucleotides at the polymorphic site of each biallelic marker tested, following the manufacturer's recommendations. After 4 minutes at 94° C., 20 PCR cycles of 15 sec at 55° C., 5 sec at 72° C., and 10 sec at 94° C. were carried out in a Tetrad PTC-225 thermocycler (MJ Research). The unincorporated dye terminators were then removed by ethanol precipitation. Samples were finally resuspended in formamide-EDTA loading buffer and heated for 2 min at 95° C. before being loaded on a polyacrylamide sequencing gel. The data were collected by an ABI PRISM 377 DNA sequencer and processed using the CanIonSCAN software (Perkin Elmer).

Following gel analysis, data were automatically processed with software that allows the determination of the alleles of biallelic markers present in each amplified fragment.

The software evaluates such factors as whether the intensities of the signals resulting from the above microsequencing procedures are weak, normal, or saturated, or whether the signals are ambiguous. In addition, the software identifies significant peaks (according to shape and height criteria). Among the significant peaks, peaks corresponding to the targeted site are identified based on their position. When two significant peaks are detected for the same position, each sample is categorized classification as homozygous or heterozygous type based on the height ratio.

### Example 5

### Preparation of Antibody Compositions to the CanIon Protein

Substantially pure protein or polypeptide is isolated from transfected or transformed cells containing an expression vector encoding the CanIon protein or a portion thereof. The concentration of protein in the final preparation is adjusted, for example, by concentration on an Amicon filter device, to the level of a few micrograms/ml. Monoclonal or polyclonal antibody to the protein can then be prepared as follows:

A. Monoclonal Antibody Production by Hybridoma Fusion

Monoclonal antibody to epitopes in the CanIon protein or a portion thereof can be prepared from murine hybridomas according to the classical method of Kohler, G. and Milstein, C., (1975) or derivative methods thereof. Also see Harlow, E., and D. Lane. 1988.

Briefly, a mouse is repetitively inoculated with a few micrograms of the CanIon protein or a portion thereof over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (1980), and derivative methods thereof. Selected positive clones can be expanded and their monoclonal antibody product harvested for use. Detailed procedures for monoclonal antibody production are described in Davis, L. et al. Basic Methods in Molecular Biology Elsevier, New York. Section 21-2.

B. Polyclonal Antibody Production by Immunization

Polyclonal antiserum containing antibodies to heterogeneous epitopes in the CanIon protein or a portion thereof can be prepared by immunizing suitable non-human animal with the CanIon protein or a portion thereof, which can be unmodified or modified to enhance immunogenicity. A suitable non-human animal is preferably a non-human mammal is selected, usually a mouse, rat, rabbit, goat, or horse. Alternatively, a crude preparation which has been enriched for CanIon concentration can be used to generate antibodies. Such proteins, fragments or preparations are introduced into the non-human mammal in the presence of an appropriate adjuvant (e.g. aluminum hydroxide, RIBI, etc.) which is known in the art. In addition the protein, fragment or preparation can be pretreated with an agent which will increase antigenicity, such agents are known in the art and include, for example, methylated bovine serum albumin (mBSA), bovine serum albumin (BSA), Hepatitis B surface antigen, and keyhole limpet hemocyanin (KLH). Serum from the immunized animal is collected, treated and tested according to known procedures. If the serum contains polyclonal antibodies to undesired epitopes, the polyclonal antibodies can be purified by immunoaffinity chromatography.

Effective polyclonal antibody production is affected by many factors related both to the antigen and the host species. Also, host animals vary in response to site of inoculations and dose, with both inadequate or excessive doses of antigen resulting in low titer antisera. Small doses (ng level) of antigen administered at multiple intradermal sites appears to be most reliable. Techniques for producing and processing polyclonal antisera are known in the art, see for example, Mayer and Walker (1987). An effective immunization protocol for rabbits can be found in Vaitukaitis, J. et al. (1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al. (1973). Plateau concentration of antibody is usually in the range of 0.1 to 0.2 mg/ml of serum (about 12 μM). Affinity of the antisera for the antigen is determined by preparing competitive binding curves, as described, for example, by Fisher, D. (1980).

Antibody preparations prepared according to either the monoclonal or the polyclonal protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

While the preferred embodiment of the invention has been illustrated and described, it will be appreciated that various changes can be made therein by the one skilled in the art without departing from the spirit and scope of the invention.

### REFERENCES

Abbondanzo S J et al., 1993, Methods in Enzymology, Academic Press, New York, pp 803-823
Ajioka R. S. et al., Am. J. Hum. Genet., 60:1439-1447, 1997
Altschul et al., 1990, J. Mol. Biol. 215(3):403-410
Altschul et al., 1993, Nature Genetics 3:266-272
Altschul et al., 1997, Nuc. Acids Res. 25:3389-3402
Ames, R. S. et al. J. Immunol. Methods 184:177-186 (1995)
Anderson et al., Symp. Soc. Exp. Biol. 48: 85-97 (1994)
Anton M. et al., 1995, J. Virol., 69: 4600-4606
Araki K et al. (1995) Proc. Natl. Acad. Sci. USA. 92(1):160-4.
Ashkenazi, A. et al. PNAS 88:10535-10539 (1991)
Aszódi et al., Proteins: Structure, Function, and Genetics, Supplement 1:38-42 (1997)
Ausubel et al. (1989) Current Protocols in Molecular Biology, Green Publishing Associates and Wiley Interscience, N.Y.
Bartunek, P. et al. Cytokine 8(1):14-20 (1996)
Baubonis W. (1993) A Nucleic Acids Res. 21(9):2025-9.
Beaucage et al., Tetrahedron Lett 1981, 22: 1859-1862
Better, M. et al. Science 240:1041-1043 (1988)
Blouin J L et al. Nature Genetics, 20: 70-73 (1998)
Bradley A., 1987, Production and analysis of chimaeric mice. In: E. J. Robertson (Ed.), Teratocarcinomas and embryonic stem cells: A practical approach. IRL Press, Oxford, pp. 113.
Brain R J et al., 1993, Mol. Cell. Biol., 13: 4760-4769
Brinkman U. et al. J. Immunol. Methods 182:41-50 (1995)
Brown E L, Belagaje R, Ryan M J, Khorana H G, Methods Enzymol 1979; 68:109-151
Brutlag et al. Comp. App. Biosci. 6:237-245, 1990
Brzustowicz et al., Am. J. Hum. Genet. 65:1096-1103 (1999)
Bulman et al., Hum. Mol. Gen. 6(10) 1679-1685 (1997)
Burton, D. R. et al. Advances in Immunology 57:191-280(1994)
Bush et al., 1997, J. Chromatogr., 777: 311-328.
Carlson, N. G. et al. J. Biol. Chem. 272(17):11295-11301 (1997)
Chai H. et al. (1993) Biotechnol. Appl. Biochem. 18:259-273.
Chee et al. (1996) Science. 274:610-614.
Chen and Kwok Nucleic Acids Research 25:347-353 1997
Chen et al. (1987) Mol. Cell. Biol. 7:2745-2752.
Chen et al. Proc. Natl. Acad. Sci. USA 94/20 10756-10761, 1997
Chen, Z. et al. Cancer Res. 58(16): 3668-3678 (1998)
Cho R J et al., 1998, Proc. Natl. Acad. Sci. USA, 95(7): 3752-3757.
Chou J. Y., 1989, Mol. Endocrinol., 3: 1511-1514.
Clark A. G. (1990) Mol. Biol. Evol. 7:111-122.
Coles R, Caswell R, Rubinsztein D C, Hum Mol Genet. 1998; 7:791-800
Compton J. (1991) Nature. 350(6313):91-92.
Davis L. G., M. D. Dibner, and J. F. Battey, Basic Methods in Molecular Biology, ed., Elsevier Press, NY, 1986
Dempster et al., (1977) J. R. Stat. Soc., 39B:1-38.
Deng, B. et al. Blood 92(6): 1981-1988 (1998)
Dent D S & Latchman D S (1993) The DNA mobility shift assay. In: Transcription Factors: A Practical Approach (Latchman D S, ed.) pp 1-26. Oxford: IRL Press
Denyer et al., Drug Disc. Today 3(7): 323-332 (1998
Eckner R. et al. (1991) EMBO J. 10:3513-3522.
Edwards et Leatherbarrow, Analytical Biochemistry, 246, 1-6 (1997)
Engvall, E., Meth. Enzymol. 70:419 (1980)
Excoffier L. and Slatkin M. (1995) Mol. Biol. Evol., 12(5): 921-927.
Feldman and Steg, 1996, Medicine/Sciences, synthese, 12:47-55
Felici F., 1991, J. Mol. Biol., Vol. 222:301-310
Fell, H. P. et al. J. Immunol. 146:2446-2452 (1991)
Fields and Song, 1989, Nature, 340 : 245-246
Fisher, D., Chap. 42 in: Manual of Clinical Immunology, 2d Ed. (Rose and Friedman, Eds.) Amer. Soc. For Microbiol., Washington, D.C. (1980)
Flotte et al. (1992) Am. J. Respir. Cell Mol. Biol. 7:349-356.
Fodor et al. (1991) Science 251:767-777.
Fraley et al. (1979) Proc. Acad. Acad. Sci. USA. 76:3348-3352.
Fried M, Crothers D M, Nucleic Acids Res 1981; 9:6505-6525
Fromont-Racine M. et al., 1997, Nature Genetics, 16(3): 277-282.
Fuller S. A. et al. (1996) Immunology in Current Protocols in Molecular Biology, Ausubel et al. Eds, John Wiley & Sons, Inc., USA.
Furth P. A. et al. (1994) Proc. Natl. Acad. Sci. USA. 91:9302-9306.
Garner M M, Revzin A, Nucleic Acids Res 1981; 9:3047-3060
Geysen H. Mario et al. 1984. Proc. Natl. Acad. Sci. U.S.A. 81:3998-4002
Ghosh and Bacchawat, 1991, Targeting of liposomes to hepatocytes, IN: Liver Diseases, Targeted diagnosis and therapy using specific receptors and ligands. Wu et al. Eds., Marcel Dekker, New York, pp. 87-104.
Gillies, S. D. et al. J. Immunol. Methods 125:191-202 (1989)
Gillies, S. O. et al. PNAS 89:1428-1432 (1992)
Gonnet et al., 1992, Science 256:1443-1445 Gonzalez et al., Drug Disc. Today 4(9): 431:439 (1999)
Gopal (1985) Mol. Cell. Biol., 5:1188-1190.
Gossen M. et al. (1992) Proc. Natl. Acad. Sci. USA. 89:5547-5551.
Gossen M. et al. (1995) Science. 268:1766-1769.
Graham et al. (1973) Virology 52:456-457.
Green et al., Ann. Rev. Biochem. 55:569-597 (1986)
Greenspan and Bona, FASEB J. 7(5):437-444 (1989)
Griffin et al. Science 245:967-971 (1989)
Grompe, M. (1993) Nature Genetics. 5:111-117.
Grompe, M. et al. (1989) Proc. Natl. Acad. Sci. U.S.A. 86:5855-5892.
Gu H. et al. (1993) Cell 73:1155-1164.
Gu H. et al. (1994) Science 265:103-106.
Guatelli J C et al. Proc. Natl. Acad. Sci. USA. 35:273-286.
Hacia J G, Brody L C, Chee M S, Fodor S P, Collins F S, Nat Genet. 1996; 14(4):441-447
Hall L. A. and Smirnov I. P. (1997) Genome Research, 7:378-388.
Hames B. D. and Higgins S. J. (1985) Nucleic Acid Hybridization: A Practical Approach.
Hames and Hamill, Pfugers Arch. 391, 85-100 (1981).
Hammerling, et al., in: MONOCLONAL ANTIBODIES AND T-CELL HYBRIDOMAS 563-681 (Elsevier, N.Y., 1981)
Harju L, Weber T, Alexandrova L, Lukin M, Ranki M, Jalanko A, Clin Chem 1993; 39(11Pt 1):2282-2287
Harland et al. (1985) J. Cell. Biol. 101:1094-1095.
Harlow, E., and D. Lane. 1988. Antibodies A Laboratory Manual. Cold Spring Harbor Laboratory. pp. 53-242
Harper J W et al., 1993, Cell, 75 : 805-816
Harrop, J. A. et al. J. Immunol. 161(4): 1786-1794 (1998)
Hawley M. E. et al. (1994) Am. J. Phys. Anthropol. 18:104.
Hell et al., Annals N.Y. Acad. Sci. 747:282-293 (1994)
Henikoff and Henikoff, 1993, Proteins 17:49-61
Higgins et al., 1996, Methods Enzymol. 266:383-402
Higgins Ed., IRL Press, Oxford.
Hillier L. and Green P. Methods Appl., 1991, 1: 124-8.
Hoess et al. (1986) Nucleic Acids Res. 14:2287-2300.
Huang L. et al. (1996) Cancer Res 56(5): 1137-1141.
Huston et al. Methods in Enzymology 203:46-88 (1991)
Huygen et al. (1996) Nature Medicine. 2(8):893-898.
Izant J G, Weintraub H, Cell 1984 April; 36(4):1007-15
Julan et al. (1992) J. Gen. Virol. 73:3251-3255.
Kanegae Y. et al., Nucl. Acids Res. 23:3816-3821 (1995).
Karlin and Altschul, 1990, Proc. Natl. Acad. Sci. USA 87:2267-2268
Kettleborough, C. A. et al. Eur. J. Immunol. 24:952-958(1994)
Khoury J. et al., Fundamentals of Genetic Epidemiology, Oxford University Press, NY, 1993
Kim U-J. et al. (1996) Genomics 34:213-218.
Klein et al. (1987) Nature. 327:70-73.
Koch, et al., J. Biol. Chem. 265 (29): 17786-17791 (1990)
Kohler, G. and Milstein, C., Nature 256:495 (1975)
Koller et al. (1992) Annu. Rev. Immunol. 10:705-730.
Kozal M J, Shah N, Shen N, Yang R, Fucini R, Merigan T C, Richman D D, Morris D, Hubbell E, Chee M, Gingeras T R, Nat Med 1996; 2(7):753-759
Lander and Schork, Science, 265, 2037-2048, 1994
Landegren U. et al. (1998) Genome Research, 8:769-776.
Lange K. (1997) Mathematical and Statistical Methods for Genetic Analysis. Springer, New York.
Lee et al., FEBS Lett. 445 231:236 (1999).
Lenhard T. et al. (1996) Gene. 169:187-190.
Liautard, J. et al. Cytokinde 9(4):233-241(1997)
Lin M W et al. Hum. Genet., 99(3): 417-420 (1997)
Linton M. F. et al. (1993) J. Clin. Invest. 92:3029-3037.
Liu Z. et al. (1994) Proc. Natl. Acad. Sci. USA. 91:4528-4262.
Livak et al., Nature Genetics, 9:341-342, 1995
Livak K J, Hainer J W, Hum Mutat 1994; 3(4):379-385
Lockhart et al. Nature Biotechnology 14: 1675-1680, 1996
Lucas A. H., 1994, In: Development and Clinical Uses of Haempophilus b Conjugate;
Manger et al., Anal. Biochem. 214: 190-194 (1993).
Mansour S. L. et al. (1988) Nature. 336:348-352.
Marshall R. L. et al. (1994) PCR Methods and Applications. 4:80-84.
McCormick et al. (1994) Genet. Anal. Tech. Appl. 11:158-164.
McLaughlin B. A. et al. (1996) Am. J. Hum. Genet. 59:561-569.
Morena et al., Annals NY Acad. Sci. 102-117 (1999)
Mori, et al., Nature 350: 398-402 (1991)
Morrison, Science 229:1202 (1985)
Morton N. E., Am. J. Hum. Genet., 7:277-318, 1955
Muller, Y. A. et al. Structure 6(9):1153-1167 (1998)
Mullinax, R. L. et al. BioTechniques 12(6):864-869 (1992)
Muzyczka et al. (1992) Curr. Topics in Micro. and Immunol. 158:97-129.
Nada S. et al. (1993) Cell 73:1125-1135.
Nagy A. et al., 1993, Proc. Natl. Acad. Sci. USA, 90: 8424-8428.
Naramura, M. et al. Immunol. Lett. 39:91-99(1994)
Narang S A, Hsiung H M, Brousseau R, Methods Enzymol 1979; 68:90-98
Neda et al. (1991) J. Biol. Chem. 266:14143-14146.
Newton et al. (1989) Nucleic Acids Res. 17:2503-2516.
Nickerson D. A. et al. (1990) Proc. Natl. Acad. Sci. U.S.A. 87:8923-8927.
Nicolau C. et al., 1987, Methods Enzymol., 149:157-76.
Nicolau et al. (1982) Biochim. Biophys. Acta. 721:185-190.
Nissinoff, J. Immunol. 147(8): 2429-2438 (1991).
Nyren P, Pettersson B, Uhlen M, Anal Biochem 1993; 208(1):171-175
O'Reilly et al. (1992) Baculovirus Expression Vectors. A Laboratory Manual. W. H. Freeman and Co., New York.
Ohno et al. (1994) Science. 265:781-784.
Oi et al., BioTechniques 4:214 (1986)
Oldenburg K. R. et al., 1992, Proc. Natl. Acad. Sci., 89:5393-5397.
Olesen et al., Voltage gated ion channel modulators, 7-8 December, Philadelphia Pa., USA (1995)
Orita et al. (1989) Proc. Natl. Acad. Sci. U.S.A. 86: 2776-2770.
Ott J., Analysis of Human Genetic Linkage, John Hopkins University Press, Baltimore, 1991
Ouchterlony, O. et al., Chap. 19 in: Handbook of Experimental Immunology D. Wier (ed) Blackwell (1973)
Padlan E. A., Molecular Immunology 28(4/5): 489-498 (1991)
Parmley and Smith, Gene, 1988, 73:305-318
Pastinen et al., Genome Research 1997; 7:606-614
Pearson and Lipman, 1988, Proc. Natl. Acad. Sci. USA 85(8):2444-2448
Pease S. and William R. S., 1990, Exp. Cell. Res., 190: 209-211.
Perlin et al. (1994) Am. J. Hum. Genet. 55:777-787.
Persic, L. et al. Gene 187 9-18 (1997)
Peterson et al., 1993, Proc. Natl. Acad. Sci. USA, 90 : 7593-7597.
Pietu et al. Genome Research 6:492-503, 1996
Pitard, V. et al. J. Immunol. Methods 205(2):177-190 (1997)
Potter et al. (1984) Proc. Natl. Acad. Sci. U.S.A. 81(22):7161-7165.
Prat, M. et al. J. Cell. Sci. 111(Pt2): 237-247 (1998)
Ramunsen et al., 1997, Electrophoresis, 18: 588-598.
Reid L. H. et al. (1990) Proc. Natl. Acad. Sci. U.S.A. 87:4299-4303.
Risch, N. and Merikangas, K. (Science, 273:1516-1517, 1996
Robertson E., 1987, Embryo-derived stem cell lines. In: E. J. Robertson Ed. Teratocarcinomas and embryonic stem cells: a practical approach. IRL Press, Oxford, pp. 71.
Roguska M. A. et al. PNAS 91:969-973), (1994)
Rossi et al., Pharmacol. Ther. 50:245-254, (1991)
Roth J. A. et al. (1996) Nature Medicine. 2(9):985-991.
Roux et al. (1989) Proc. Natl. Acad. Sci. U.S.A. 86:9079-9083.
Ruano et al. (1990) Proc. Natl. Acad. Sci. U.S.A. 87:6296-6300.
Sambrook, J., Fritsch, E. F., and T. Maniatis. (1989) Molecular Cloning. A Laboratory Manual. 2ed. Cold Spring Harbor Laboratory, Cold Spring Harbor, N.Y.
Samson M, et al. (1996) Nature, 382(6593):722-725.
Samulski et al., (1989) J. Virol. 63:3822-3828.
Sanchez-Pescador R. (1988) J. Clin. Microbiol. 26(10): 1934-1938.
Sarkar, G. and Sommer S. S. (1991) Biotechniques.
Sauer B. et al. (1988) Proc. Natl. Acad. Sci. U.S.A. 85:5166-5170.
Sawai, H. et al. AJRI 34:26-34 (1995)
Schedl A. et al., 1993a, Nature, 362: 258-261.
Schedl et al., 1993b, Nucleic Acids Res., 21: 4783-4787.
Schena et al. Science 270:467-470, 1995
Schena et al., 1996, Proc Natl Acad Sci USA, 93(20):10614-10619.
Schneider et al. (1997) Arlequin. A Software For Population Genetics Data Analysis. University of Geneva.
Schwartz and Dayhoff, eds., 1978, Matrices for Detecting Distance Relationships: Atlas of Protein Sequence and Structure, Washington: National Biomedical Research Foundation
Sczakiel G. et al. (1995) Trends Microbiol. 3(6):213-217.
Shay J. W. et al., 1991, Biochem. Biophys. Acta, 1072: 1-7.
Sheffield, V. C. et al. (1991) Proc. Natl. Acad. Sci. U.S.A. 49:699-706.
Shizuya et al. (1992) Proc. Natl. Acad. Sci. U.S.A. 89:8794-8797.
Shoemaker D D, et al., Nat Genet. 1996; 14(4):450-456
Shu, L. et al. PNAS 90:7995-7999 (1993)
Skerra, A. et al. Science 240:1038-1040. (1988)
Smith (1957) Ann. Hum. Genet. 21:254-276.
Smith et al. (1983) Mol. Cell. Biol. 3:2156-2165.
Sosnowski R G, et al., Proc Natl Acad Sci USA 1997; 94: 1119-1123
Sowdhamini et al., Protein Engineering 10:207, 215 (1997)
Spielmann S, and Ewens W. J., Am. J. Hum. Genet., 62:450-458, 1998
Spielmann S. et al., Am. J. Hum. Genet., 52:506-516, 1993
Sternberg N. L. (1992) Trends Genet. 8:1-16.
Sternberg N. L. (1994) Mamm. Genome. 5:397-404.
Stryer, L., Biochemistry, 4th edition, 1995
Studnicka G. M. et al. Protein Engineering 7(6):805-814(1994)
Syvanen A C, Clin Chim Acta 1994; 226(2):225-236
Szabo A. et al. Curr Opin Struct Biol 5, 699-705 (1995)
Tacson et al. (1996) Nature Medicine. 2(8):888-892.
Taryman, R. E. et al. Neuron 14(4):755-762 (1995) Schaid D. J. et al., Genet. Epidemiol., 13:423-450, 1996
Te Riele et al. (1990) Nature. 348:649-651.
Terlau et al., Naturwissenschaften 85: 437-444 (1998)
Terwilliger J. D. and Ott J., Handbook of Human Genetic Linkage, John Hopkins University Press, London, 1994
Thomas K. R. et al. (1986) Cell. 44:419-428.
Thomas K. R. et al. (1987) Cell. 51:503-512.
Thompson et al., 1994, Nucleic Acids Res. 22(2):4673-4680
Tur-Kaspa et al. (1986) Mol. Cell. Biol. 6:716-718.
Tyagi et al. (1998) Nature Biotechnology. 16:49-53.
Urdea M. S. (1988) Nucleic Acids Research. 11:4937-4957.
Urdea M. S. et al. (1991) Nucleic Acids Symp. Ser. 24:197-200.
Vaitukaitis, J. et al. J. Clin. Endocrinol. Metab. 33:988-991 (1971)
Valadon P., et al., 1996, J. Mol. Biol., 261:11-22.
Van der Lugt et al. (1991) Gene. 105:263-267.
Vil, H. et al. PNAS 89:11337-11341 (1992)
Vlasak R. et al. (1983) Eur. J. Biochem. 135:123-126.
Wabiko et al. (1986) DNA. 5(4):305-314.
Walker et al. (1996) Clin. Chem. 42:9-13.
Wang et al., 1997, Chromatographia, 44: 205-208.
Weir, B. S. (1996) Genetic data Analysis II: Methods for Discrete population genetic Data, Sinauer Assoc., Inc., Sunderland, Mass., U.S.A.
Westerink M. A. J., 1995, Proc. Natl. Acad. Sci., 92:4021-4025
White, M. B. et al. (1992) Genomics. 12:301-306.
White, M. B. et al. (1997) Genomics. 12:301-306.
Williams, et al. Science 257: 389-395 (1992)
Wong et al. Gene. 10:87-94 (1980)
Wood S. A. et al., 1993, Proc. Natl. Acad. Sci. USA, 90: 4582-4585.
Wu and Wu (1987) J. Biol. Chem. 262:4429-4432.
Wu and Wu (1988) Biochemistry. 27:887-892.
Wu et al. (1989) Proc. Natl. Acad. Sci. USA. 86:2757.
Yagi T. et al. (1990) Proc. Natl. Acad. Sci. U.S.A. 87:9918-9922.
Yoon, D. Y. et al. J. Immunol. 160(7): 3170-3179 (1998)
Zhang et al., Neuropharmacology 32 (11): 1075-1088 (1993)
Zhao et al., Am. J. Hum. Genet., 63:225-240, 1998
Zheng, X. X. et al. J. Immunol. 154:5590-5600 (1995)
Zhu, Z. et al. Cancer Res. 58(15): 3209-3214 (1998)
Zou Y. R. et al. (1994) Curr. Biol. 4:1099-1103.

What is claimed:

1. An isolated, purified, or recombinant polynucleotide comprising:
a) the nucleotide sequence of SEQ ID No: 1, 2, 3, 4, or 6;
b) a polynucleotide sequence complementary to SEQ ID No: 1, 2, 3, 4, or 6;
c) a contiguous span of at least 50 nucleotides of SEQ ID No 4, wherein said polynucleotide encodes a biologically active CanIon polypeptide; or
d) a polynucleotide sequence that encodes a human CanIon polypeptide comprising the amino acid sequence of SEQ ID No 5, or a biologically active fragment of a human CanIon polypeptide.

2. The polynucleotide of claim 1, attached to a solid support.

3. The polynucleotide of claim 2, wherein said polynucleotide is arranged in an array on said solid support.

4. The polynucleotide of claim 3, wherein said array is addressable.

5. The polynucleotide of claim 1, further comprising a label.

6. The polynucleotide of claim 2, further comprising a label.

7. A recombinant vector comprising the polynucleotide of claim 1.

8. The recombinant vector of claim 7, wherein said polynucleotide of claim 1 is operably linked to a promoter.

9. A host cell comprising the recombinant vector of claim 7.

10. A non-human host animal or mammal comprising the recombinant vector of claim 7.

11. A mammalian host cell or non-human host mammal comprising a polynucleotide according to claim 1.

12. An isolated, purified, or recombinant polypeptide comprising the amino acid sequence of SEQ ID No: 5.

13. A method of making a polypeptide, said method comprising:
a) providing a population of cells comprising a polynucleotide encoding the polypeptide according to claim 12 operably linked to a promoter;
b) culturing said population of cells under conditions conducive to the production of said polypeptide within said cells; and
c) purifying said polypeptide from said population of cells.

14. A method of binding an anti-CanIon antibody to a CanIon polypeptide comprising contacting said antibody with a polypeptide according to claim 12 under conditions in which said antibody can specifically bind to said polypeptide.

15. A method of detecting the expression of a CanIon gene within a cell, said method comprising the steps of:
a) contacting said cell or an extract from said cell with either of:
i) a polynucleotide that hybridizes under stringent conditions to a polynucleotide of claim 1; or
ii) a polypeptide that specifically binds to the CanIon polypeptide; and
b) detecting either: a) the presence or absence of hybridization between said polynucleotide and an RNA species within said cell or extract; or b) the presence or absence of binding of said polypeptide to a protein within said cell or extract;
wherein a detection of the presence of said hybridization or of said binding indicates that said CanIon gene is expressed within said cell.

16. The method of claim 15, wherein said polynucleotide is an oligonucleotide primer, and wherein said hybridization is detected by detecting the presence of an amplification product comprising the sequence of said primer.

17. The method of claim 15, wherein said polypeptide is an anti-CanIon antibody.

18. A method of identifying a candidate modulator of a CanIon polypeptide, said method comprising:
a) contacting the polypeptide according to claim 12 with a test compound; and
b) determining whether said compound specifically binds to said polypeptide;
wherein a detection that said compound specifically binds to said polypeptide indicates that said compound is a candidate modulator of said CanIon polypeptide.

19. The method of claim 18, further comprising testing the activity of said CanIon polypeptide in the presence of said candidate modulator, wherein a difference in the activity of said CanIon polypeptide in the presence of said candidate modulator in comparison to the activity in the absence of said candidate modulator indicates that the candidate modulator is a modulator of said CanIon polypeptide.

20. A method of identifying of modulator of a CanIon polypeptide, said method comprising:
a) contacting the polypeptide according to claim 12 with a test compound; and
b) detecting the activity of said polypeptide in the presence and absence of said compound;
wherein a detection of a difference in said activity in the presence of said compound in comparison to the activity in the absence of said compound indicates that said compound is a modulator of said CanIon polypeptide.

21. The method of claim 19, wherein said polypeptide is present in a cell or cell membrane, and wherein said activity comprises voltage gated ion channel activity.

22. The method of claim 20, wherein said polypeptide is present in a cell or cell membrane, and wherein said activity comprises voltage gated ion channel activity.

23. A method for the preparation of a pharmaceutical composition comprising
a) identifying a modulator of a CanIon polypeptide; and
b) combining said modulator with a physiologically acceptable carrier.
